(19) United States
(12) Patent Application Publication
(10) Pub. No.: US 2026/0103690 A1
(43) Pub. Date: Apr. 16, 2026

(54) PROGRAMMABLE DNA TRANSPOSASES FOR NUCLEIC ACID MANIPULATION

(71) Applicants: Arc Research Institute, Palo Alto, CA (US); The Regents of the University of California, Oakland, CA (US)

(72) Inventors: Nicholas PERRY, Palo Alto, CA (US); Matthew DURRANT, Palo Alto, CA (US); Patrick HSU, Palo Alto, CA (US)

(21) Appl. No.: 19/134,588

(22) PCT Filed: Dec. 1, 2023
(86) PCT No.: PCT/US2023/082192

§ 371 Date: May 30, 2025

Related U.S. Application Data
(60) Provisional application No. 63/385,736, filed on Dec. 1, 2022.
(60) Provisional application No. 63/581,208, filed on Sep. 7, 2023.

(51) Int. Cl. C12N 9/12; C12N 15/11; C12N 15/90

(52) U.S. Cl. CPC C12N 9/1241; C12N 15/11; C12N 15/907

(57) ABSTRACT

The invention relates to a novel system for nucleic acid engineering utilizing components of IS110 family transposons. IS110 transposases encoded by IS110 elements were identified to utilize an RNA sequence, termed the bridgeRNA, which targets donor and target sequence sites for polynucleotide recombination reactions. In certain aspects, the application relates to the utilization and reprogramming of the bridgeRNA to direct IS110 transposases to integrate sequences at predetermined sites. Programmable insertion, excisive recombination, and/or inversion enables the integration or transposition of any polynucleotide sequence encoding a donor site or target site recognized by the IS110 transposase into any other polynucleotide sequence containing a target site sequence or donor site sequence, respectively, using an IS110 transposase and bridgeRNA. The invention has applications in cellular engineering, genome engineering, genetic medicine, synthetic biology, molecular diagnostics, transgenic organisms, and biological research.

This International Patent Application claims the benefit of and priority to U.S. Application No. 63/385,736, filed Dec. 1, 2022, entitled “INSERTION OF CARGO WITH PROGRAMMABLE TRANSPOSASES,” and U.S. Application No. 63/581,208, filed Sep. 7, 2023, entitled “PROGRAMMABLE DNA TRANSPOSASES FOR NUCLEIC ACID MANIPULATION,” the content of which are hereby incorporated by reference in their entirety.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a “lengthy” Sequence Listing which has been submitted via DVD-R in lieu of a printed paper copy, and is hereby incorporated by reference in its entirety. Said DVD-R, recorded on Dec. 1, 2023, are labeled “CRF”, “Copy 1” and “Copy 2”, respectively, and each contains only one identical 819,898,112 bytes (781 MB) file (2220476-00121WO1_SL.xml).

### BACKGROUND OF THE INVENTION

DNA sequence manipulation, namely insertion, inversion, and excision of DNA, is a foundational capability underpinning synthetic biology, genome engineering, cell engineering, and genetic medicine. Such reactions have been utilized for decades, such as the Cre-loxP system, which relies on the Cre protein's specificity for pre-engineered loxP DNA sequences. The orientation and location of these DNA sequences relative to one another are used to achieve insertion, inversion, or excision of long DNA sequences. More modern methods, especially for DNA insertion, often utilize random, semi-random, or non-programmable site-specific enzymes such as recombinases and transposases for manipulation of DNA sequences, relying on enzyme-encoded specificity for target sequences. RNA-guided programmable nucleases such as Cas9, have been utilized to excise DNA by cutting at more than one site, albeit relying on DNA repair of the resulting double stranded break. For insertion and inversion, the amino acid-encoded DNA specificities of these systems are difficult to reprogram without significant protein engineering efforts, in particular through the tethering of a separate programmable targeting domain, such as an RNA-guided CRISPR effector. Other methods utilize synthetic or naturally occurring reprogrammable transposition systems that require the assembly of multiple protein and nucleic acid subunits to achieve DNA integration, yet delivery of these systems to cells can be complex and they require extensive engineering to achieve efficient inversion or excision of DNA sequences. There is, therefore, significant need and potential for a simple DNA sequence recombination system that is reprogrammable without protein engineering and easily delivered to cells.

### SUMMARY OF THE INVENTION

It is understood that any of the embodiments described below can be combined in any desired way, and that any embodiment or combination of embodiments can be applied to each of the aspects described below, unless the context indicates otherwise.

In certain aspects, the invention provides a recombinant nucleic acid editing system comprising: a) an IS110 family transposase, or a nucleic acid comprising a sequence encoding the IS110 family transposase; and b) a nucleic acid comprising a sequence encoding a bridgeRNA.

In some embodiments, the nucleic acid comprising a sequence encoding a bridgeRNA comprises a left end (LE) sequence of a transposon that encodes the IS110 family transposase. In some embodiments, the nucleic acid comprising a sequence encoding a bridgeRNA comprises a right end (RE) sequence of a transposon that encodes the IS110 family transposase.

In some embodiments, the recombinant nucleic acid editing system further comprises a nucleic acid comprising a RE sequence and a LE sequence or a RE sequence, a core sequence, and a LE sequence of the IS110 element that encodes the IS110 family transposase. In some embodiments, the recombinant nucleic acid editing system further comprises a nucleic acid comprising a right flank (RF) sequence and a left flank (LF) sequence or a RF sequence, a core sequence, and a LF sequence of the target site sequence for the IS110 family transposase. In some embodiments, the nucleic acid comprising the RE sequence and the LE sequence or the RE sequence, the core sequence, and the LE sequence further comprises a nucleic acid sequence for insertion into a target site sequence. In some embodiments, the target site sequence comprises a RF sequence and a LF sequence or a RF sequence, a core sequence, and a LF sequence for the IS110 family transposase. In some embodiments, the nucleic acid comprising the RF sequence and the LF sequence or the RF sequence, the core sequence, and the LF sequence further comprises a nucleic acid sequence for insertion into a donor site sequence. In some embodiments, the donor site sequence comprises a RE sequence and a LE sequence or a RE sequence, a core sequence, and a LE sequence of the IS110 element that encodes the IS110 family transposase.

In some embodiments, the bridgeRNA comprises a nucleotide sequence at least 50% identical to a bridgeRNA sequence of SEQ ID NOS: 1-348 or SEQ ID NOS: 349-10175. In some embodiments, said RE sequence comprises a RE sequence of SEQ ID NOS: 1-348, 30354-30529, 349-10175 or 30530-40356, said LE sequence comprises a LE sequence of SEQ ID NOS: 1-348, 30354-30529, 349-10175 or 30530-40356, and/or said core sequence comprises a core sequence of SEQ ID NOS: 1-348, 30354-30529, 349-10175 or 30530-40356.

In certain aspects, the invention provides a recombinant nucleic acid editing system comprising: a) an IS110 family transposase, or a nucleic acid comprising a sequence encoding the IS110 family transposase; and b) a bridgeRNA, or a nucleic acid comprising a sequence encoding the bridgeRNA, the bridgeRNA comprising at least one stem-loop structure and further comprising at least one internal loop comprising a first nucleotide sequence that is complementary to a first target site sequence of a target DNA, a second nucleotide sequence that is complementary to a second target site sequence which is on the opposite strand of the target DNA to the first target site sequence and wherein the bridgeRNA is capable of forming a complex with the IS110 family transposase.

In some embodiments, the bridgeRNA further comprises a third nucleotide sequence that is complementary to a first donor site sequence of a donor DNA, and a fourth nucleotide sequence that is complementary to a second donor site sequence which is on the opposite strand of the donor DNA to the first donor site sequence. In some embodiments, the third nucleotide sequence and the fourth nucleotide sequence are on a second internal loop.

In some embodiments, the IS110 family transposase comprises a RuvC-like DEDD catalytic domain and a transposase domain. In some embodiments, the IS110 family transposase further comprises a linker domain between the RuvC-like DEDD catalytic domain and transposase domain. In some embodiments, the linker domain comprises a coiled-coil linker domain. In some embodiments, the RuvC-like DEDD catalytic domain comprises an amino acid sequence at least 50% identical to a RuvC-like DEDD catalytic domain sequence of SEQ ID NOs: 10176-10523, 10524-20350, or 40357-516430. In some embodiments, the IS110 family transposase comprises a RuvC-like DEDD catalytic domain that forms a similar tertiary structure to the RuvC-like DEDD catalytic domain of IS621. In some embodiments, the IS110 family transposase RuvC-like DEDD catalytic domain comprises a tertiary structure similar to a tertiary structure of the RuvC-like DEDD catalytic domain of IS621 if the template modeling score (TM-score) for the RuvC-like DEDD catalytic domain of the IS110 family transposase is 0.5 or higher. In some embodiments, the RuvC-like DEDD catalytic domain comprises an amino acid sequence at least 15% identical to a RuvC-like DEDD catalytic domain sequence of SEQ ID NOs: 10176-10523, 10524-20350, or 40357-516430.

In some embodiments, the transposase domain comprises an amino acid sequence at least 50% identical to a transposase domain sequence of SEQ ID NOs: 10176-10523, 10524-20350, or 40357-516430. In some embodiments, the IS110 family transposase comprises a transposase domain that forms a similar tertiary structure to the transposase domain of IS621. In some embodiments, the IS110 family transposase domain comprises a tertiary structure similar to a tertiary structure of the transposase domain of IS621 if the template modeling score (TM-score) for the transposase domain of the IS110 family transposase is 0.5 or higher. In some embodiments, the transposase domain comprises an amino acid sequence at least 15% identical to a transposase domain sequence of SEQ ID NOs: 10176-10523, 10524-20350, or 40357-516430.

In some embodiments, the IS110 family transposase comprises an amino acid sequence at least 50% identical to SEQ ID NOs: 10176-10523, 10524-20350, or 40357-516430. In some embodiments, the IS110 family transposase further comprises a tertiary structure similar to a tertiary structure of IS621. In some embodiments, the IS110 family transposase comprises a tertiary structure similar to a tertiary structure of IS621 if the template modeling score (TM-score) for the transposase is 0.5 or higher. In some embodiments, the transposase domain comprises an amino acid sequence at least 15% identical to a transposase domain sequence of SEQ ID NOs: 10176-10523, 10524-20350, or 40357-516430.

In some embodiments, the IS110 family transposase is an IS110 group transposase. In some embodiments, the IS110 family transposase is an IS1111 group transposase. In some embodiments, the IS1111 group transposase is IS1111A or IS1111_229727. In some embodiments, wherein the IS110 group transposase is IS621, ISPa11, IsPa29, ISMmg1, ISPfl1, ISMae40, ISStma6, ISAzs32, ISMex9, ISCARN28, ISAar16, ISCps7, ISPpu9, ISRe19, ISEsa2, ISMma5, IS900, or ISHne5. In some embodiments, the IS110 group transposase comprises an amino acid sequence at least 50% identical to IS621 (SEQ ID NO: 10176).

In some embodiments, the bridgeRNA comprises at least two stem-loop structures comprising a first stem-loop and a second stem-loop where the first stem-loop is 5′ to the second stem-loop and wherein the first stem-loop comprises a target binding loop and the second stem-loop comprises a donor binding loop. In some embodiments, the bridgeRNA further comprises a third stem-loop structure 5′ of the first stem-loop. In some embodiments, the stem of the first stem-loop is 5 to 35 nucleotides long and the loop is 3-10 nucleotides long, the target binding loop is 5 to 20 nucleotides long, the stem of the second stem-loop is 5 to 35 nucleotides long and the loop is 3-10 nucleotides long, and the donor binding loop is 5 to 20 nucleotides long. In some embodiments, the stem of the second stem-loop structure comprises 1 to 4 loops or bubbles that are each 1 to 10 nucleotides long.

In some embodiments, the bridgeRNA comprises a nucleotide sequence comprising any of the 5′ to 3′ sequences provided in FIG. 19, wherein “n” represents any nucleotide, “R” represents an A or G nucleotide, and “Y” represents a C or U nucleotide. In some embodiments, the bridgeRNA comprises a 5′ to 3′ secondary structure provided in the first row, second row, third row, or fourth row of secondary structure for said sequence provided in FIG. 19, wherein matching parentheses “(“and”)” indicate base-paired nucleotides, and “.” indicates unpaired bases.

In some embodiments, the bridgeRNA comprises a stem-loop structure as depicted in FIG. 2D, FIG. 11B, or FIG. 13.

In some embodiments, the target binding loop of the bridgeRNA comprises: a left-target guide (LTG) comprising, in the 5′ to 3′ direction, a nucleotide sequence complementary to first strand of a target site sequence wherein the 3′ end of the LTG is complementary to at least one of the nucleotides of a core sequence on the first strand of the target site sequence; and a right-target guide (RTG) comprising, in the 5′ to 3′ direction, a nucleotide sequence that is a reverse complement to an opposite strand of the first strand of the target site sequence wherein the 3′ end of the RTG is reverse complementary to at least one of the nucleotides of the core sequence on the opposite strand of the first strand of the target site sequence; wherein the target site sequence is a polynucleotide sequence; and/or wherein the donor binding loop of the bridgeRNA comprises: a left-donor guide (LDG) comprising, in the 5′ to 3′ direction, a nucleotide sequence complementary to a first strand of a donor site sequence wherein the 3′ end of the LDG complementary to at least one of the nucleotides of a core sequence on the first strand of the donor site sequence; and a right-donor guide (RDG) comprising, in the 5′ to 3′ direction, a nucleotide sequence that is a reverse complement to an opposite strand of the first strand of the donor site sequence wherein the 3′ end of the RDG is reverse complementary to at least one of the nucleotides of the core sequence on the opposite strand to the first strand of the donor site sequence; wherein the donor site sequence is a polynucleotide sequence, and the core sequence of the donor site sequence is the same as the core sequence of the target site sequence.

In some embodiments, the target binding loop of the bridgeRNA comprises: a left-target guide (LTG) comprising, in the 5′ to 3′ direction, a nucleotide sequence is reverse complementary to an opposite strand to a first strand of a target site sequence wherein the 3′ end of the LTG is complementary to at least one of the nucleotides of a core sequence on the opposite strand to the first strand of the target site sequence; and a right-target guide (RTG) comprising, in the 5′ to 3′ direction, a nucleotide sequence that is a complementary to the first strand of the target site sequence wherein the 3′ end of the RTG is complementary to at least one of the nucleotides of the core sequence on the first strand of the target site sequence; wherein the target site sequence is a polynucleotide sequence; and/or wherein the donor binding loop of the bridgeRNA comprises: a left-donor guide (LDG) comprising, in the 5′ to 3′ direction, a nucleotide sequence complementary to a first strand of a donor site sequence wherein the 3′ end of the LDG complementary to at least one of the nucleotides of a core sequence on the first strand of the donor site sequence; and a right-donor guide (RDG) comprising, in the 5′ to 3′ direction, a nucleotide sequence that is a reverse complement to an opposite strand of the first strand of the donor site sequence wherein the 3′ end of the RDG is reverse complementary to at least one of the nucleotides of the core sequence on the opposite strand to the first strand of the donor site sequence; wherein the donor site sequence is a polynucleotide sequence, and the core sequence of the donor site sequence is the same as the core sequence of the target site sequence.

In some embodiments, the target site sequence comprises sequence X₁X₂X₃X₄X₅X₆X₇X₈X₉X₁₀X₁₁X₁₂X₁₃X₁₄ where X is any nucleotide, and X₈X₉ are the core and one or more of X₁₂, X₁₃, and X₁₄ are optionally part of the target site sequence. In some embodiments, the bridgeRNA encodes an LTG in the 5′ to 3′ direction X₁X₂X₃X₄X₅X₆X₇X₈ and an RTG in the 5′ to 3′ direction Y₁₄Y₁₃Y₁₂Y₁₁Y₁₀Y₉Y₈ where Y is the complementary nucleotide to X and one or more of Y₁₄, Y₁₃, and Y₁₂ are optionally part of RTG or wherein the bridgeRNA encodes an LTG in the 5′ to 3′ direction X₁X₂X₃X₄X₅X₆X₇X₈X₉ and an RTG in the 5′ to 3′ direction Y₁₄Y₁₃Y₁₂Y₁₁Y₁₀Y₉ where Y is the complementary nucleotide to X and one or more of Y₁₄, Y₁₃, and Y₁₂ are optionally part of RTG. In some embodiments, the donor site sequence comprises sequence STIR-Xₙ₁—X₁X₂X₃X₄X₅X₆X₇X₈X₉X₁₀X₁₁X₁₂X₁₃X₁₄—Xₙ₂-STIR where X is any nucleotide, one or more of X₁₂, X₁₃, and X₁₄ are optionally part of the donor site sequence, STIR is optional, but if present is a sub-terminal inverted repeat comprising 2 to 20 nucleotides, and X₈X₉ are the core, and n1 and n2 can independently be zero to 10. In some embodiments, the bridgeRNA encodes an LDG in the 5′ to 3′ direction X₁X₂X₃X₄X₅X₆X₇X₈ and an RDG in the 5′ to 3′ direction Y₁₄Y₁₃Y₁₂Y₁₁Y₁₀Y₉Y₈ where Y is the complementary nucleotide to X and one or more of Y₁₄, Y₁₃, and Y₁₂ are optionally part of RDG or wherein the bridgeRNA encodes an LDG in the 5′ to 3′ direction X₁X₂X₃X₄X₅X₆X₇X₈X₉ and an RDG in the 5′ to 3′ direction Y₁₄Y₁₃Y₁₂Y₁₁Y₁₀Y₉ where Y is the complementary nucleotide to X and one or more of Y₁₄, Y₁₃, and Y₁₂ are optionally part of RDG. In some embodiments, the STIR, if present, comprises a G/T rich nucleotide sequence. In some embodiments, the 5′ STIR, if present, comprises a G/T rich nucleotide sequence.

In some embodiments, the target site sequence is located on genomic DNA, a linear dsDNA, a dsDNA plasmid, ssDNA or RNA. In some embodiments, the donor site sequence is located on genomic DNA, a linear dsDNA, a dsDNA plasmid, ssDNA or RNA. In some embodiments, the dsDNA plasmid further comprises a polynucleotide sequence for insertion into the donor site sequence. In some embodiments, the dsDNA plasmid further comprises a polynucleotide sequence for insertion into the target site sequence. In some embodiments, the target site sequence and donor site sequence on the genomic DNA are located on the same DNA strand. In some embodiments, the target site sequence and donor site sequence on the genomic DNA are located on different chromosomes.

In some embodiments, the bridgeRNA is a split bridgeRNA. In some embodiments, the bridgeRNA comprises a first RNA molecule that comprises a first portion of the bridgeRNA and a second RNA molecule that comprises a second portion of the bridgeRNA. In some embodiments, the first portion of the bridgeRNA comprises the internal loop comprising the first nucleotide sequence that is complementary to the first target site sequence of the target DNA and the second portion of the bridgeRNA comprises the second internal loop comprising the third nucleotide sequence that is complementary to the first donor site sequence of a donor DNA, and the fourth nucleotide sequence that is complementary to the second donor site sequence. In some embodiments, the first portion of the bridgeRNA is encoded on a nucleic acid and is operably linked to a first promoter and the second portion of the bridgeRNA is encoded on the same or a different nucleic acid and is operably linked to a second promoter. In some embodiments, the first portion of the bridgeRNA and second portion are encoded on a nucleic acid and further comprise one or more ribozyme cites which results in cleavage of the expressed bridgeRNA into the first and second RNA molecules.

In some embodiments, any of the LTG, RTG, LDG, and/or RDG of the bridgeRNA are complementary to more nucleotides their respective target site sequence or donor site sequence than the number of complementary nucleotides in its corresponding naturally occurring bridgeRNA. In some embodiments, the total number of nucleotides comprising the target binding loop and/or donor binding loop of the bridgeRNA is the same as its corresponding naturally occurring bridgeRNA. In some embodiments, the total number of nucleotides comprising the target binding loop and/or donor binding loop of the bridgeRNA is the increased as compared to its corresponding naturally occurring bridgeRNA. In some embodiments, any of the LTG, RTG, LDG, and/or RDG of the bridgeRNA are not complementary to a nucleotide of the core sequence in their respective target site sequence or donor site sequence but are complementary to the same number of nucleotides in the respective target site sequence or donor site sequence as its corresponding naturally occurring bridgeRNA. In some embodiments, the target site sequence comprises sequence X₋₁X₁X₂X₃X₄X₅X₆X₇X₈X₉X₁₀X₁₁X₁₂X₁₃X₁₄ where X is any nucleotide, and X₈X₉ are the core and one or more of X₁₂, X₁₃, and X₁₄ are optionally part of the target site sequence and wherein the bridgeRNA encodes an LTG in the 5′ to 3′ direction X₋₁X₁X₂X₃X₄X₅X₆X₇ and an RTG in the 5′ to 3′ direction Y₁₄Y₁₃Y₁₂Y₁₁Y₁₀Y₉Y₈ where Y is the complementary nucleotide to X and one or more of Y₁₄, Y₁₃, and Y₁₂ are optionally part of RTG. In some embodiments, the donor site sequence comprises sequence STIR-Xₙ₁—X₁X₂X₃X₄X₅X₆X₇X₈X₉X₁₀X₁₁X₁₂X₁₃X₁₄—Xₙ₂-STIR where X is any nucleotide, one or more of X₁₂, X₁₃, and X₁₄ are optionally part of the donor site sequence, STIR is optional, but if present is a sub-terminal inverted repeat comprising 2 to 20 nucleotides, and X₈X₉ are the core, and n1 and n2 can independently be 1 to 10, and wherein the bridgeRNA encodes an LDG in the 5′ to 3′ direction X₁X₁X₂X₃X₄X₅X₆X₇ and an RDG in the 5′ to 3′ direction Y₁₄Y₁₃Y₁₂Y₁₁Y₁₀Y₉Y₈ where Y is the complementary nucleotide to X and one or more of Y₁₄, Y₁₃, and Y₁₂ are optionally part of RDG. In some embodiments, one or more of the nucleotides of the LTG, RTG, LDG, and/or RDG of the bridgeRNA base pair to a nucleotide of their respective target site sequence or donor site sequence vis non-canonical base pairing.

In certain aspects, the invention provides a vector comprising any of the nucleic acids of the nucleic acid editing system of the invention. In certain aspects, the invention provides a host cell comprising any of the vector(s) of the invention. In some embodiments, any of the nucleic acids of the nucleic acid editing system further comprise an inducible promoter.

In certain aspects, the invention provides a method of integrating a DNA molecule of interest into a sequence specific site of a DNA of interest of a cell, the method comprising introducing into the cell: a nucleic acid editing system of the invention.

In some embodiments, the cell is a mammalian cell. In some embodiments, the cell is a human cell.

In some embodiments, the DNA of interest of the cell comprises a donor site sequence and the DNA molecule of interest is part of a nucleic acid of the nucleic acid editing system comprising a target site sequence. In some embodiments, the DNA of interest of the cell comprises a target site sequence and the DNA molecule of interest is part of a nucleic acid of the nucleic acid editing system comprising a donor site sequence. In some embodiments, the DNA of interest of the cell further comprises a second donor site sequence and the DNA molecule of interest further comprises a second target site sequence and the nucleic acid editing system comprises a second bridgeRNA that targets the second donor site sequence and second target site sequence. In some embodiments, the DNA of interest of the cell comprises a second target site sequence and the DNA molecule of interest further comprises a second donor site sequence and the nucleic acid editing system comprises a second bridgeRNA that targets the second donor site sequence and second target site sequence. In some embodiments, the sequence of the bridgeRNA was engineered before introduction of the nucleic acid editing system to bind to the donor site sequence and target site sequence. In some embodiments, the DNA of interest of the cell is the genome of the cell. In some embodiments, the DNA of interest of the cell is a plasmid.

In certain aspects, the invention provides a method of inverting a DNA sequence of a DNA of interest of a cell, the method comprising introducing into the cell: a nucleic acid editing system of the invention, wherein a target site sequence and donor site sequence are present on the same DNA molecule of interest and the LD of the donor site sequence and RT of the target site sequence are on the same DNA strand.

In some embodiments, the DNA of interest of the cell is the genome of the cell. In some embodiments, the sequence of the bridgeRNA was engineered, before introduction of the nucleic acid editing system, to bind to the donor site sequence and target site sequence.

In certain aspects, the invention provides a method of excising a DNA sequence of a DNA of interest of a cell, the method comprising introducing into the cell: a nucleic acid editing system of the invention, wherein a target site sequence and donor site sequence are present on the same DNA molecule of interest and the LD of the donor site sequence and LT of the target site sequence are on the same DNA strand.

In some embodiments, the DNA of interest of the cell is the genome of the cell. In some embodiments, the sequence of the bridgeRNA was engineered, before introduction of the nucleic acid editing system, to bind to the donor site sequence and target site sequence.

In certain aspects, the invention provides a method of translocating DNA sequences between two linear DNA molecules of interest, the method comprising introducing into a cell: a nucleic acid editing system of the invention, wherein a donor site sequence is present on a first linear DNA molecule and a target site sequence is present on a second linear DNA molecule.

In some embodiments, the linear DNA molecules of interest of the cell are chromosomes of the cell. In some embodiments, the sequence of the bridgeRNA was engineered, before introduction of the nucleic acid editing system, to bind to the donor site sequence and target site sequence.

Other embodiments of the invention are further described in the following sections of the application, including the Drawings, Detailed Description, Examples, and Claims. Still other objects and advantages of the invention will become apparent by those of skill in the art from the disclosure herein, which are simply illustrative and not restrictive. Thus, other embodiments will be recognized by the ordinarily skilled artisan without departing from the spirit and scope of the invention.

### BRIEF DESCRIPTION OF THE DRAWINGS

The patent or application file contains at least one drawing originally executed in color. To conform to the requirements for PCT patent applications, many of the figures presented herein are black and white representations of images originally created in color.

FIGS. 1A-E show general features of IS110 insertion sequence elements. (A) Sequence features of the two groups of IS110s. The IS110 group is characterized by longer left non-coding ends (LE) and shorter right non-coding ends (RE). The IS1111 group is characterized by shorter LE and longer RE. In both groups, a core sequence motif (1-5 nt) is found at both ends of the element. IS110s were previously thought to lack sub-terminal inverted repeats (STIRs), while IS111 is were known to have 6-12 nt sub-terminal inverted repeats. However, as described herein, most IS110s also have short STIRs (see FIGS. 10A-B). IS110 elements are typically 1000-2000 nt in length. Figure discloses SEQ ID NOS 795157-795160. (B) Depiction of domains of IS110 transposases. IS110 transposases are typically 300-500aa long. The RuvC-like domain (DEDD_Tnp_IS110 by Pfam) includes a canonical DEDD catalytic motif. The IS110 Tnp domain (Transposase_20 by Pfam) has a catalytic serine. (C) Depiction of IS110 element life-cycle. Genomically integrated IS110 elements cut themselves from the genome and results in scarless repair of the genomic DNA target site and the formation of a circular IS110 element. In the circular form, the RE becomes adjacent to the LE. Insertion can occur into the same dsDNA target site or into new target sites. An inserted linear IS110 element consists of a left non-coding end (LE), coding sequence for a transposase (Tpase), and a right non-coding end (RE). The inserted IS110 element is flanked on the left end with a left flank (LF) (leftmost box) comprising a left target (LT) sequence and on the right end with a right flank (RF) (rightmost box) comprising a right target (RT) sequence. For many IS110 elements, between the LF and LE and between RE and RF are identical “core” sequences (rhombus), although not all IS110 elements may utilize a “core” sequence. IS110 elements excise themselves, resulting in a pre-insertion (“target”) site bearing LF-core (if present)-RF, and a circular element with RE-core (if present)-LE-Tpase. Concatenation of the RE-LE junction forms a “donor” site sequence as a subsequence of the RE-LE junction, which, if present, includes the other core sequence found on the integrated element. The donor site sequence may also include sub-terminal inverted repeats (STIR) indicated with triangles, although STIRs are not required for IS110 recombinase activity. Concatenation of the RE-LE also forms a promoter which may promote expression of a bridgeRNA from the RE or LE. It may also promote expression of the transposase. The circular form of the element can reinsert into the target site from which it was excised or into any other polynucleotide with a target site sequence; the bridgeRNA encoded within the LE or RE recognizes the donor site sequence and/or the target site sequence to mediate transposition. Figure discloses SEQ ID NOS 795161-795164. (D) IS110 transposase phylogenetic tree. IS110s have several clades but are largely distinguished by the IS110 and IS1111 groups. Showing host kingdom and phylum to demonstrate diverse origins. The location of notable IS110 transposases is highlighted on the tree. (E) Comparison of IS110 group end lengths. IS110s typically have LEs longer than their REs, while IS111 s typically have REs longer than their LEs.

FIGS. 2A-D show identification of the bridgeRNA from the model IS110 IS621. (A) RNAseq of IS110 non-coding ends (SEQ ID NO: 795165). A plasmid encoded RE-LE sequence was delivered to E. coli and RNA was extracted and sequenced. Boundaries of an RNA encoded within the LE are defined across 6 orthologs of IS621. (B) Demonstration of bridgeRNA binding to IS621 transposase. The RNA in part A for IS621 was purified and exposed to IS621 transposase at varying concentrations. Microscale thermophoresis is used to measure the binding kinetics of the bridgeRNA to the transposase. A scrambled RNA with no bases matching and a reverse complement of the bridgeRNA serve as negative controls. (C) Determination of IS621 bridgeRNA structure. Hundreds of related LEs of IS621 were aligned and RNA structure was predicted for each. The predominant structure at each position in the alignment was calculated and plotted; structures were characterized as 5′ stem, 3′ stem, hairpin, other or gap. A structure between the LE start and CDS start emerged that features a consensus bridgeRNA structure and accessory structure on the 5′ end. (D) Depiction of IS621 bridgeRNA structure: nnnnnnnnYYnRRnnnnYYYnnnYnnnnRRnnnnYYGGAYGCCGYnYYnRnCCUnnRRYnnn ARYYYGYnnYGUAGAUnnnYGCRnCRRnYRYYnnnnnnnnYnnGYnnnRRRYCGRACnG nAUCnYnGGCYGGYnnnYCGRnARYCYGCAUYACAAGUnGRUnRCRYRAnnnn (SEQ ID NO: 795166). The information in C is represented here using the software R2R. The target binding loop and donor binding loop are labeled. An accessory structure for this particular bridgeRNA is also labeled. The secondary structure of IS621 bridgeRNA structure in FIG. 2D is represented in “dot-bracket” notation as: ...((((((((((....)))))))))).((((((((((.(((............(((((....)))))..............))))))).......(.((((....(((............. ((((((....)))))...).............)))).)... where matching parentheses “(“and”)” indicate base pairs, and unpaired bases are shown as dots (“.”).

FIGS. 3A-D show prediction and verification of the mechanism of bridgeRNA recognition of DNA. (A) Depiction of covariation analysis approach. Boundaries of IS110 elements are identified using comparative genomics. The non-coding ends are concatenated as they would be in the circular form of an IS110 to identify the donor sites and the pre-integration target sites are extracted. bridgeRNA sequences are predicted from non-coding ends. Alignments of target sites (or donor sites) are compared to structurally informed alignments of bridgeRNA sequences to identify covarying positions (SEQ ID NOS 795167-795168, respectively, in order of appearance). (B) Covariation between the bridgeRNA of IS621 and its target and donor. A covariation score as calculated by the software CCMpred was normalized and plotted for each position of the target and donor along the left end. Subsequences of the bridgeRNA, covary with the target and donor. Covarying sequences are observed to be complementary or reverse-complementary to the donor and target. These covarying regions in the bridgeRNA were then inspected for evidence of base-pairing with the target and donor sequences to identify the programmable guide sequences. Figure discloses SEQ ID NOS 795172, 795164, and 795169-795171, respectively, in order of appearance. (C) Model of IS621 bridgeRNA with target and donor sequences. A representation of the R2R structure in FIG. 2D is shown with the LTG, RTG, LDG, and RDG positions within the target binding loop and donor binding loop. The LT, RT, LD, and RD are shown with their relative positions to the core sequence found in both the target and donor. Sub-terminal inverted repeats are also shown on the donor. The target binding loop of the bridgeRNA comprises a left-target guide (LTG) and right-target guide (RTG) which are specific for sequences of the target site, i.e., the left target (LT) and right target (RT) sequences, respectively. For IS110 family transposases that use a core sequence, both the LTG and RTG may include base-pairing specificity for at least one base of the core dinucleotide sequence (dashed lines). The donor binding loop of the bridgeRNA comprises a left-donor guide (LDG) and right-donor guide (RDG) which are specific for sequences of the donor site, i.e., the left donor (LD) and right donor (RD) sequences, respectively. For IS110 family transposases that use a core sequence, both the LDG and RDG may include base-pairing specificity for at least one base of the core sequence. The donor site may also encode sub-terminal inverted repeats (STIR) which interact directly with the transposase protein, and therefore may be required for the transposition reaction or other parts of the IS110 life-cycle such as cutting and pasting. In some embodiments, additional bridgeRNA nucleotides outside of these described guide sequences can play a role in programming specificity for different target and donor sequences. Figure discloses SEQ ID NOS 795172 and 795164, respectively, in order of appearance. (D) Demonstration of sequence specific binding of target and donor. IS621 transposase and bridgeRNA ribonucleoproteins were exposed to the WT target and donor sequences as well as scrambled DNA sequences that match at 0 positions. The binding affinity for the WT bridgeRNA for the WT donor and target is shown.

FIG. 4 shows a diagram of how to reprogram a bridgeRNA to recognize new targets and donors. Depiction of bridgeRNAs programmed to recognize new targets and donors. The WT bridgeRNA is first depicted with the WT target and donor sequence. The target and/or donor loops are modified in each of the following examples. The bridgeRNA sequences are depicted with the LTG, RTG, LDG, and RDG sequences indicated which are capable of base-pairing with the target and donor site sequences depicted. These subsequences may be reprogrammed to bind with any desired target or donor sequence. In all five examples, the core nucleotides of the target and donor match each other. In the final example, the STIRs are modified to any sequence, since they are not strictly required for transposition function. Figure discloses SEQ ID NOS 795169, 795173-795174, 795170-795171, 795175-795178, 795171, 795179, 795176, 795180, 795178, 795180-795184, 795183, 795185-795186, 795187-795188, and 795187, respectively, in order of appearance.

FIGS. 5A-D shows in cellulo demonstration of transposition and target reprogramming. (A) GFP reporter assay for transposition. A pDonor plasmid encodes an inactive GFP gene adjacent to the RE-LE of IS621, which expresses the bridgeRNA. pDonor is co-transformed into E. coli along with pTarget, which encodes the WT target and the IS621 transposase, both of which are adjacent to promoters. Integration of donor into target activates GFP expression. (B) Demonstration of GFP reporter assay function. E. coli cells are measured for GFP expression on the FITC channel using flow cytometry. GFP expression is observed using the assay in (A) only when the transposase is WT; inactivation of conserved residues in either the RuvC-like domain or Tnp domain abolishes transposition. (C) Diagram of reprogramming bridgeRNAs to recognize new targets. The target loop of the bridgeRNA is modified to recognize a new target. The target is also modified to match the target loop when used in the reporter assay in A. Graph shows demonstration of specific re-targeting of transposition to new targets. Plasmids expressing seven bridgeRNAs with unique target loops were paired with matching targets or the WT target. Transposition is observed only when the matching target is provided, as measured by flow cytometry for GFP expression. Figure discloses SEQ ID NOS 795189, 795190, 795176, 795191-795194, and 795173, respectively, in order of appearance. (D) Separation of the bridgeRNA from the RE-LE. The bridgeRNA can be separated and expressed from a separate promoter to achieve higher rates of transposition. The donor sequence can be reduced from 298 bp to at least 22 bp without affecting transposition efficiency. Truncation to 11 bp (removing the STIRs) reduces transposition efficiency to near background. Some integrations are observed when the bridgeRNA is present with the 11 bp donor. Systems lacking a bridgeRNA never result in integration. Figure discloses SEQ ID NOS 795195-795196, respectively, in order of appearance.

FIGS. 6A-E shows IS621 bridgeRNA target/target loop mismatch tolerance and reprogramming screen. (A) Schematic depicting antibiotic resistance reporter design. A minimal donor (22 bp) is encoded on a plasmid adjacent to a kanamycin resistance gene. A second plasmid encodes the target, bridgeRNA, and transposase. The target is linked to the bridgeRNA using a barcode. Recombination between the donor and target plasmid results in E. coli survival, and functional bridgeRNA target loop and target pairs are recorded using next generation sequencing. (B) Schematic depicting target specificity screen design. The target (SEQ ID NOS 795197-795205, respectively, in order of appearance) and target loop are varied, except for the core of the target and the subsequences of the LTG and RTG that bind the core. The donor loop (and donor) are held constant. Target and target loop pairs are designed to assay single mismatches, double mismatches, and total mismatches. Targets in the screen are selected to reduce the number of off-targets in the E. coli genome. (C) Abundance of target and target loop pairs. Abundance is measured by barcode counts per million reads. Target/target loop pairs with zero mismatches are generally more abundant, while increasing the number of mismatches decreases abundance. (D) Sequence logo of top quintile of targets. The relative enrichment of nucleotides at each position of the target are shown for target/target loop pairs with zero mismatches in the top quintile of 6364 target/target loop pairs. (E) Single mismatch tolerance by position. The relative enrichment of nucleotides for the top quintile of target sets are shown when the target loop does or does not mismatch for each position of the target (SEQ ID NO: 795173). The best performing zero-mismatch pair in each target set is used to represent the set, and the top quintile of target sets is shown.

FIGS. 7A-H show IS621 bridgeRNA donor/donor loop mismatch tolerance and reprogramming screen. (A) Schematic depicting antibiotic resistance reporter design. A full length donor is encoded on a plasmid adjacent to a kanamycin resistance gene. The constitutive promoter of the WT system expresses the bridgeRNA. A unique molecular identifier (UMI) identifies a donor/donor loop pair. A second plasmid encodes the target and transposase. Recombination between the donor and target plasmid results in E. coli survival, and functional bridgeRNA donor loop and donor pairs are recorded using next generation sequencing. (B) Schematic depicting donor specificity and reprogramming design. The donor (SEQ ID NOS 795206-795212, respectively, in order of appearance) and donor loop are varied, except the core and the subsequences of the LDG and RDG that bind the core. The target loop (and target) are held constant, but the target is a non-WT sequence not found in the E. coli genome. Target and target loop pairs are designed to assay single mismatches and double mismatches for the WT donor sequence. 5000 random perfectly-matched donor and donor loops are also assayed. (C) UMI abundance of donor/donor loop pairs with 1 nt difference from WT. Counts are plotted for pairs with 0 or 1 mismatches between donor and donor loop. UMI abundance of WT donor paired with WT donor loop is depicted as a red dashed line. (D) UMI abundance of donor/donor loop pairs with 2 nt difference from WT. Counts are plotted for pairs with 0 or 2 mismatches between donor and loop. UMI abundance of WT donor paired with WT donor loop is depicted as a red dashed line. (E) UMI abundance of donor/donor loop pairs with 0 mismatches. CPM values of donor/donor loop pairs are binned by the number of nucleotide differences between the reprogrammed donor and WT donor. CPM of WT donor paired with the WT donor loop is depicted as a red dashed line. (F) Single mismatch tolerance by position in the donor (SEQ ID NO: 795196). The relative enrichment of nucleotides for the top quintile of donor sets is shown, with all 4×4=16 mismatch combinations tested at each position in the donor. (G) Sequence logo of top quintile of donors. The relative enrichment of nucleotides at each position of the target are shown for donor-donor loop pairs with zero mismatches in the top quintile of 5000 donor/donor loop pairs. (H) Demonstration of specific re-targeting of transposition to new donors. Plasmids expressing five bridgeRNAs with unique donor loops (SEQ ID NOS 795196 and 795213-795221, respectively, in order of appearance) were matched with cognate donors or the WT donor. Transposition is observed only when the matching donor is provided, as measured by flow cytometry for GFP expression via FITC. Results were generated using a 22 bp donor using the approach in FIG. 5D.

FIGS. 8A-C show a diagram and demonstration of DNA rearrangements with IS621 transposase. (A) Depiction of GFP-reporter assay for DNA insertion. A plasmid encoding a donor and a GFP coding sequence and a plasmid encoding a target plasmid adjacent to a promoter are delivered into E. coli. Co-expression of a bridgeRNA encoding target and donor loops matching the provided target and donor results in efficient insertion in E. coli. Figure discloses SEQ ID NOS 795164 and 795222-795224, respectively, in order of appearance. (B) Depiction of GFP-reporter assay for excisive recombination of DNA. A plasmid encoding a promoter adjacent to a donor and a target preceded by a terminator and followed by a GFP coding sequence is delivered to E. coli. Co-expression of a bridgeRNA encoding target and donor loops matching the provided target and donor results in efficient excisive recombination in E. coli; removal of the intervening sequence encoding the terminator enables GFP expression. The reaction results in one DNA molecule becoming two DNA molecules. Figure discloses SEQ ID NOS 795164, 795225, 795223, and 795226, respectively, in order of appearance. (C) Depiction of GFP-reporter assay for inversion of DNA. A plasmid encoding a promoter adjacent to a donor and a target preceded by a terminator and GFP coding sequence is delivered to E. coli, Co-expression of a bridgeRNA encoding target and donor loops matching the provided target and donor results in efficient inversion in E. coli; inversion of the sequence between the donor and target enables GFP expression. Figure discloses SEQ ID NOS 795164, 795227, 795226, and 795228, respectively, in order of appearance. (A-C) Insertion efficiency is measured by the percent of cells expressing GFP as measured by flow cytometry. Excisive recombination efficiency is measured by the percent of cells expressing GFP as measured by flow cytometry. Inversion efficiency is measured by the percent of cells expressing GFP as measured by flow cytometry.

FIGS. 9A-B shows a diagram and demonstration of DNA insertion into the E. coli genome with IS621 transposase. (A) Depiction of genome integration assay. An E. coli cell line containing a donor plasmid is made that can grow under kanamycin selection below 37° C. due to kanamycin resistance encoded on the donor plasmid. At 37° C., the donor plasmid cannot replicate. A pHelper plasmid encoding IS621 transposase and a bridgeRNA that recognizes the donor on the plasmid and a target in the genome is delivered to E. coli. Growth at 37° C. results in selection for E. coli that have integrated the plasmid into the genome, which is required for survival. (B) Integration profile using bridgeRNAs targeting four sites in the genome for integration. Targets are rank ordered by relative abundance of integration locations from nanopore sequencing data. Integration sites are colored by the number of differences between the observed target site and the expected target site. Figure discloses SEQ ID NOS 795229-795235, respectively, in order of appearance.

FIGS. 10A-B shows identification of sub-terminal inverted repeats in IS110 group IS110 elements. (A) Diagram of approach for identifying sub-terminal inverted repeats. Boundaries of IS110 elements are identified using comparative genomics and BLAST. The non-coding ends are concatenated as they would be in the circular form and are aligned. Covarying sequences are compared across the donor up to 25 bp in each direction from the core. (B) Covariation of sequences within the donor identifies short sub-terminal inverted repeats. A covariation score is plotted for each position of the donor for covariation with itself.

FIGS. 11A-C shows prediction and verification of a bridgeRNA expressed from the RE of an IS1111. (A) Determination of IS1111_229727 bridgeRNA structure. Hundreds of related REs of IS1111_229727 were aligned and RNA structure was predicted for each. The predominant structure at each position in the alignment was calculated and graphed; structures were characterized as 5′, 3′ stem, hairpin, other or gap. A structure between the estimated RE start and element boundary emerges that features a target binding loop and donor binding loop. (B) Depiction of IS1111_229727 bridgeRNA structure nnnnnGnYYnYYGRnRGnGRCGYRGCCCGGYnnnnGnGYAAYCCnCGnnnnYRYnnnGnR RCnRnnnYnRAYYYnCGnnnYYAGAnnGnGGCAGGCnCnYnnGRCGGAnnYnnGYRnnG YGGUAnCCARYCCRCGRAURUCAGCnnGAUYnRCCGUCGnnnnnnRCYnGCYnCGYC nCnnYYRRnnRnYnnnnn (SEQ ID NO: 795236). The information in A is represented here using the software R2R. The secondary structure of IS1111_229727 bridgeRNA structure in FIG. 11B is represented in “dot-bracket” notation as: ...(((((((((((.(((((((((((..(((....((((...((((..........(((((((((.))))..)))))..........))))...))))....(((((..........(((((..... ...))))).................))))).......))).))))))))))).)))))))))))... where matching parentheses “(“and”)” indicate base pairs, and unpaired bases are shown as dots (“.”). (C) RNAseq verification of IS1111_229727 bridgeRNA. RNAseq coverage is represented over the RE of IS1111_229727.

FIGS. 12A-B show alignment of RuvC and Tnp domains of diverse IS110 transposases. (A) Alignment of IS110 RuvC-like domains (SEQ ID NOS 795237-795261, respectively, in order of appearance). Alignment is depicted with conserved residues and regions. Residues are colored by amino acid chemical properties. (B) Alignment of IS110 Tnp domains (SEQ ID NOS 795262-795286, respectively, in order of appearance). Alignment is depicted with conserved residues and regions. Residues are colored by amino acid chemical properties.

FIG. 13 shows diverse predicted bridgeRNA structures associated with diverse IS110 transposases. Showing diverse bridgeRNA consensus structures predicted from across diverse IS110 transposases. The procedure to generate each structure was the same as the procedure used to generate the IS621 bridgeRNA consensus structure. RNA covariance models were clustered using a graph-clustering approach, and consensus structures from 12 different clusters are shown. At least one loop resembling the target and/or donor loop is present in each structure. Significantly co-varying base-pairs are shown with a gray box highlight. The bridgeRNA structures in FIG. 13 are representations of SEQ ID NOs: 795287-795303, respectively, with gap positions excluded and trimming of extra unstructured bases.

FIGS. 14A-H shows tertiary structure alignment and analysis of IS110 transposase proteins. (A) Formula for the Template modeling score (TM-score), where L_{target} is the length of the amino acid sequence of the target protein, and L_{common} is the number of residues that appear in both the template and target structures. dᵢ is the distance between the ith pair of residues in the template and target structures, and dₒ(L_{target})=1.24∛(L_target−15)−1.8 is a distance scale that normalizes distances (Zhang and Skolnick 2004). Alternatively, the score can be normalized according to the length of the query protein, or the score can be normalized by the averaged length of the two proteins. A TM-score has a value in (0,1], and a cutoff of >0.5 is commonly used for identifying proteins with homologous tertiary structures (Zhang and Skolnick 2005). (B) TM-score distribution when aligning predicted IS110 structures to the IS621 AlphaFold structure. Each row shows the distribution of TM-scores when normalized according to the length described on the right—the average of the two lengths, the length of IS621, or the length of the query protein. The dotted line indicates a TM-score of 0.5, a commonly used minimum score threshold for identifying homologous proteins. (C) Structural alignment of two distantly related IS110 proteins. IS621 is shown in green, a separate predicted IS110 transposase structure is shown in cyan. Four different angles of the same structural alignment are shown. These two proteins are 18.1% at the amino acid level, but have a TM-score of 0.805. (D) TM-score distribution of IS110 structures when clustered and aligned to the IS621 structure. Protein structures were clustered at 100%, 90%, and 50% identity and a representative of each cluster was taken. The TM-score normalized by the average length of the two sequences is shown. Each panel is a different level of percent amino acid identity clustering. (E) TM-scores of RuvC and Tnp domains when aligned to IS621 domains, compared with the full protein TM-scores. Domains were extracted using the boundaries identified by the corresponding Pfam domains (DEDD_Tnp_IS110 and Transposase_20). These domain sub-structures were then aligned to the IS621 sub-structures using TM-align. TM-scores are shown for the full protein, the RuvC domain, and the Tnp domain. (F) IS630 transposase TM-scores vs. IS110 transposase TM-scores. All IS110 family and IS630 family transposase structures were aligned to the IS621 AlphaFold structure. The TM-score normalized by the average length of the two sequences is shown. The IS630 family was selected for comparison because it had a similar protein length distribution to that of IS110. (G) Schematic demonstrating the location of conserved residues within their respective protein structural domains and the estimated distances between them. On the top panel, showing the 5 conserved residues in the RuvC domain in a representative IS110 structure and a representative IS1111 structure. Residues are colored and labeled with the color red. The 5 positions are labeled P1-P5. Also showing the distances between these residues that are subsequently calculated, including D1-D3, which are colored and labeled as blue, purple, and green, respectively. On the bottom panel, showing the same but for the 5 conserved positions in the Tnp domain and the 3 calculated distances. Distances are with respect to the alpha carbon of each residue. (H) Distances between conserved residues in the RuvC and Tnp domains of IS110 AlphaFold structures. Distances were calculated as described in the previous paragraph. Showing here the distribution of distances in angstroms (Å) for each distance within each domain. See FIG. 14G as a reference for the distances.

FIG. 15 provides sequence listings for IS110 elements (SEQ ID NOs: 1-348). Elements are represented as 5′-3′ nucleotide sequences in typical FASTA format with additional formatting to indicate subsequences of interest. When available, the annotations include: Dark gray highlighting at the beginning of the sequence indicates the core. The core is only shown once and it is always on the 5′ end when annotated. Light gray highlighting indicates the LE and the RE, which always flank the CDS sequence. This is simply defined as the sequence that comes between the CDS and the core or the end of the element. The CDS sequence is shown as non-highlighted sequence with a single underline. The bridgeRNA boundary predictions are shown with lower-case nucleotides. When present, guide sequences are shown with bold typeface. When present, the 4 bold sub-sequences represent the LTG, the RTG, the LDG, and the RDG, in that order. Additional IS110 elements are provided as SEQ ID NOs: 349-10175 of the accompanying sequence listing, which is hereby incorporated by reference in its entirety. The sequence listing includes start and stop positions for Core, LE, RE, CDS, and bridgeRNA sequences as features of the sequence listing.

FIG. 16 provides sequence listings for transposase proteins described herein (SEQ ID NOs: 10176-10523). Proteins are also represented as amino acid sequences in typical FASTA format, with an extra line to represent the secondary structure predictions of each residue. Additional formatting is used to indicate subsequences of interest. When available, the annotations include: Dark gray highlighting to identify the boundaries of the RuvC-like domain as predicted using the DEDD_Tnp_IS110 Pfam domain. Light gray highlighting to identify the boundaries of the Tnp domain as predicted using the Transposase_20 Pfam domain. Bold typeface indicates amino acids that are highly conserved, with up to 5 such amino acids in each domain. The secondary structure prediction was generated using the standard mkdssp tool on all available IS110 transposase AlphaFold structures. These secondary structures were then projected onto sequences in our collection by primary sequence alignment. The different characters indicate: H, Alphahelix; B, Betabridge; E, Strand; G, Helix_3; I, Helix_5; P, Helix_PPII; T, Turn; S, Bend; “-”, Loop. These secondary structures can be used to orient a person of skill in the art, and be used to identify the coiled-coil linking domain. Additional transposase protein sequences are provided as SEQ ID NOs: 10524-20350 and 40357-516430 of the accompanying sequence listing, which is hereby incorporated by reference in its entirety. The sequence listing includes start and stop positions for RuvC-like domain and Tnp domain, as well as the P1-P5 positions for each domain used in the AlphaFold analysis, as features of the sequence listing.

FIG. 17 provides sequence listings for donors (SEQ ID NOs:30354-30529). Donors are represented as 50 nt 5′-3′ nucleotide sequences in typical FASTA format with additional formatting to indicate subsequences of interest. When available, the annotations include: Light gray highlighting indicates the right end (RE) and left end (LE), where the RE is 5′ to the core sequence, and the LE is 3′ to the core sequence. The core sequence is represented as non-highlighted text with a single underline. When present, the programmable portions of the donor that correspond with the bridgeRNA LDG and RDG are shown with bold typeface. The programmable portion of the donor RE that corresponds with the bridgeRNA LDG is referred to as the left donor (LD) and the programmable portion of the donor LE that corresponds with the bridgeRNA RDG is referred to as the right donor (RD). Additional donor sequences are provided as SEQ ID NOs: 30530-40356 of the accompanying sequence listing, which is hereby incorporated by reference in its entirety. The sequence listing includes start and stop positions for Core, LE, and RE sequences as features of the sequence listing.

FIG. 18 provides sequence listings for targets (SEQ ID NOs: 20351-20526). Targets are represented as 50 nt 5′-3′ nucleotide sequences in typical FASTA format with additional formatting to indicate subsequences of interest. When available, the annotations include: Light gray highlighting indicates the left flank (LF) and right flank (RF), where the LF is 5′ to the core sequence, and the RF is 3′ to the core sequence. The core sequence is represented as non-highlighted text with a single underline. The programmable portions of the target that correspond with the bridgeRNA LTG and RTG are shown with bold typeface. The programmable portion of the target LF that corresponds with the bridgeRNA LTG is referred to as the left target (LT) and the programmable portion of the donor RF that corresponds with the bridgeRNA RTG is referred to as the right target (RT). Additional target sequences are provided as SEQ ID NOs: 20527-30353 of the accompanying sequence listing, which is hereby incorporated by reference in its entirety. The sequence listing includes start and stop positions for Core, LF, and RF sequences as features of the sequence listing.

FIG. 19 provides consensus sequences and structures for bridgeRNA sequences (SEQ ID NOs: 795156, 795304, 795287, 795305-795328, 795297, 795329, 795330-795344, 795289, 795345-795351, 795294, 795352-795400, 795291, 795401-795412, 795300, 795288, 795302, 795413-795427, 795301, 795428-795440, 795296, 795441-795446, 795295, 795447, 795290, 795448-795454, 795298, 795455-795459, 795292, 795460-795468, 795293, 795469, 795470-795471, 795370, 795472-795508, 795299, 795509, 795510-795514, 795303, 795515-795542, 795536, and 795543-795564, respectively, in order of appearance). The name of each model is specified by lines that begin with “>”, just as in a typical FASTA file. The next line is a consensus sequence for the model, where “n” represents any nucleotide, “R” represents an A or G nucleotide, “Y” represents a C or U nucleotide, and then A, C, G, and T represent individual nucleotides. The next four lines indicate 4 possible RNA secondary structures using different confidence thresholds when running the ConsAliFold RNA structure prediction algorithm. These four lines correspond to the gamma parameters 4, 8, 16, and 32, respectively, with increasing gamma values representing more permissive models (allowing for more structure). The notation used for the secondary structure is referred to as “dot-bracket” notation, where matching parentheses “(“and”)” indicate base pairs, and unpaired bases are shown as dots (“.”).

FIG. 20 shows the IS621 transposase AlphaFold model used in the structural analysis. All available IS110 transposase AlphaFold structures were aligned back to this model using the TM-align algorithm to generate TM-scores. This analysis established that a TM-score cutoff of 0.5 is both sensitive and precise for identifying IS110 transposases.

FIG. 21 shows RuvC-like DEDD catalytic domain motifs for IS110 transposases belonging to the IS110 group.

FIG. 22 shows motifs for the “D” region of the canonical DEDD catalytic motif for IS110 transposases belonging to the IS110 group. SEQ ID NOs are shown in parentheses.

FIG. 23 shows motifs for the “E” region of the canonical DEDD catalytic motif for IS110 transposases belonging to the IS110 group. SEQ ID NOs are shown in parentheses.

FIG. 24 shows motifs for the “DD” region of the canonical DEDD catalytic motif for IS110 transposases belonging to the IS110 group. SEQ ID NOs are shown in parentheses.

FIG. 25 shows RuvC-like DEDD catalytic domain motifs for IS110 transposases belonging to the IS1111 group.

FIG. 26 shows motifs for the “D” region of the canonical DEDD catalytic motif for IS110 transposases belonging to the IS1111 group. SEQ ID NOs are shown in parentheses.

FIG. 27 shows motifs for the “E” region of the canonical DEDD catalytic motif for IS110 transposases belonging to the IS1111 group. SEQ ID NOs are shown in parentheses.

FIG. 28 shows motifs for the “DD” region of the canonical DEDD catalytic motif for IS110 transposases belonging to the IS1111 group. SEQ ID NOs are shown in parentheses.

FIG. 29 shows transposase domain motifs for IS110 transposases belonging to the IS110 group.

FIG. 30 shows motifs for the first conserved region of the transposase domain for IS110 transposases belonging to the IS110 group. SEQ ID NOs are shown in parentheses.

FIG. 31 shows motifs for the second conserved region of the transposase domain for IS110 transposases belonging to the IS110 group. SEQ ID NOs are shown in parentheses.

FIG. 32 shows transposase domain motifs for IS110 transposases belonging to the IS1111 group.

FIG. 33 shows motifs for the first conserved region of the transposase domain for IS110 transposases belonging to the IS1111 group. SEQ ID NOs are shown in parentheses.

FIG. 34 shows motifs for the second conserved region of the transposase domain for IS110 transposases belonging to the IS1111 group. SEQ ID NOs are shown in parentheses.

In FIGS. 21-34 the motifs are in common Prosite format, where x is any amino acid and x(n) represents n number of any amino acid and x(n,m) represents n to m number of any amino acids. The order is by prevalence of the domain in the transposase sequence database. In FIGS. 22-24, 26-28, 30-31, and 33-34, the motifs are shown as a list with each motif separated by a semi-colon.

FIGS. 35A-B show additional examples of predicted bridgeRNA secondary structures with predicted LTG, RTG, LDG, and RDG guide sequences. (A) Showing a schematics of 6 bridgeRNA consensus structures derived from 3 IS110 group elements and 3 IS1111 group elements. IS110 group elements typically encode their bridgeRNA in the 5′ non-coding end (LE) of the element, while IS1111 group elements typically encode their bridgeRNA in the 3′ non-coding end (RE). Guide sequences are colored according to the sequence they bind, whether it be the target (blue), the donor (orange), or the core (green). For some members of the IS1111 group, the donor-binding guide sequences are often found within a large multi-loop structure rather than an internal loop. (B) A more detailed representation of the same structures and sequences found in (A). Consensus secondary structures are shown with the IUPAC nucleotide codes circles, colored according to conservation. Highlighted guide sequences are displayed above their corresponding targets (SEQ ID NOs: 798527, 798529, 798531, 798533, 798535, 798537, respectively, in order of appearance) and donors (SEQ ID NOs: 798528, 798530, 798532, 798534, 798536, 798538 respectively, in order of appearance) for comparison. LTG, RTG, LDG, and RDG are directly labeled. The bridgeRNA structures are representations of SEQ ID NOs: 795344, 795370, 795303, 795295, 795293, 795287 with gap positions excluded and trimming of extra unstructured bases.

FIGS. 36A-E show the utility of extending the natural length of the right target guide (RTG) to increase efficiency and specificity of programmable recombination. (A) Schematic depicting how a longer RTG can be reprogrammed, in addition to how cores can be reprogrammed in conjunction with reprogramming a longer RTG. (B) Relative recombination rate between donors and targets with reprogrammed cores and 4 bp or 7 bp homology RTGs. The assay detailed in FIG. 5D was used. Results depict that having longer RTG homology enhances efficiency of recombination with the WT core sequences and reprogrammed core sequences. (C) Schematic depicting approach for genome integration, identical in approach to FIG. 9A. (D) On- and off-target integration frequency using 4 base or 7 base RTGs for targeting. The same bridgeRNAs as FIG. 9B were utilized to integrate a donor cargo into the E. coli genome with either a 4 base or 7 base RTG. Integration sites were binned by the number of differences from the 11 bp target site sequence intended by the programmed bridgeRNA with a 4 base RTG. (E) Rank order of integration sites averaged over two replicates. The same data as (D) are depicted by the relative number of integrations. High frequency integrations are highlighted by depicting their sequence and to show how the relative targeting specificity is modified when comparing a 4 base and 7 base RTG.

FIGS. 37A-E show assessment of donor boundaries of an IS110 bridge recombinase system. (A) Schematic for assaying sequence preference upstream of the donor sequence. The 6 nucleotides upstream of the LD are varied. Recombination is selected for using Kanamycin resistance and successful recombinants are measured via next-generation sequencing (NGS). Target is SEQ ID NO: 798549 and donor is SEQ ID NO:798547. (B) Schematic for assaying sequence preference downstream of the donor sequence. The 8 nucleotides downstream of the 4th position of the RD are varied, including part of the RD. Assay parameters are otherwise identical to those shown in A. Target is SEQ ID NO: 798549 and donor is SEQ ID NO:798548. (C) Nucleotide requirement upstream and downstream of the donor sequence. The 5′ and 3′ STIR sequences are highlighted in pink. Figures shows SEQ ID NO: 795196. (D-E) Sequence preference upstream (D) and downstream (E) of the donor sequence. The 5′ and 3′ STIR sequences are highlighted in pink.

FIGS. 38A-C show plasmid-plasmid recombination in human cells. (A) Schematic of plasmid-plasmid recombination assay in human cells. pEffector expressed the bridgeRNA and the recombinase from U6 and Ef1a promoters, respectively. pDonor and pTarget are recombined upon co-transfection with pEffector. PCR of the LT-RD junction with primers F and R detect recombination. (B) Verification of plasmid-plasmid recombination. PCR of the LT-RD junction is performed with only pDonor and pTarget, with pEffector lacking a bridgeRNA, and pDonor, pTarget, and pEffector. The recombinase on pEffector was evaluated with three different NLS formats. The recombinase shown is the IS621 recombinase with a bridgeRNA specific for its wild-type donor sequence and a reprogrammed target sequence Target 01. The target binding loop RTG encodes 7 bp of homology to the target. (C) Sanger sequencing confirmation of recombination. Sanger sequencing traces are aligned to the entire PCR of the LT-RD junction (top), with a zoomed-in version showing the nucleotides proximal to the LT-RD (bottom). Figure shows SEQ ID NOS: 798550, 798550, and 798551 in order of appearance.

FIGS. 39A-D shows plasmid inversion in human cells with diverse orthologs. (A) Schematic of plasmid inversion recombination assay in human cells. pEffector expresses the bridgeRNA and the recombinase from U6 and Ef1a promoters, respectively. The recombinase is fused to a P2A self cleaving peptide and EGFP to measure recombinase expression. PCR of the LT-RD junction with primers F and R detect recombination. (B) Verification of plasmid inversion recombination. PCR of the LT-RD junction is performed in the presence and absence of bridgeRNA for three different NLS configurations for IS621_23122 recombinase. (C) Percentage of cells expressing EGFP 72 hours post-transfection. Four IS110 orthologs are shown each with different NLS configurations. (D) Percentage of mCherry+ cells within the EGFP+ cell population. Four IS110 orthologs are shown each with different NLS configurations. The WT target and donor sequence are recombined for each ortholog. For IS621_127209 and IS621_23122, the sequence flanking the WT 4 nt RT was modified to allow 7 bp between the RT and the WT RTG.

FIGS. 40A-D shows bridgeRNA engineering for improved efficiency and specificity. (A) Schematic of the IS110 element IS621_23122 indicating approximate bridgeRNA boundary locations. A bridgeRNA of 179 nt (bRNA179) spans the start of the bridgeRNA to the end of the LE of the element. A bridgeRNA of 260 nt (bRNA260) starts at the same location and extends into the CDS of the recombinase. (B) Bridge editing efficiency of an inversion reporter using different length bridgeRNAs. Extending the bridgeRNA to 260 nt of natural sequence context increases efficiency relative to the 179 nt bridgeRNA. (C) Schematic comparing a WT target binding loop to an LTG-shifted target binding loop. LTG shifting allows targeting of a 16 nt target sequence by binding the 9 bp before the core rather than 9 bases including the core, increasing specificity. Target is SEQ ID NO: 798552 and donor is SEQ ID NO:798553. (D) Bridge editing efficiency of an inversion reporter with a WT bridgeRNA and an LTG-shifted bridgeRNA. Both bridgeRNAs utilize the additional 81 nt added to the 3′ end of the bridgeRNA in panel b.

FIGS. 41A-C show engineering of the human genome by delivery of a large DNA cargo and a bridge editor. (A) Schematic depicting bridge editing of the human genome via delivery of a donor plasmid. A recombinase and bridgeRNA specific for the plasmid donor (pDonor, 4.8 kb) and the target sequence in the genome results in integration of the donor into the genome. PCR of the LT-RD junction with primers F and R detect recombination. (B) PCR detection of LT-RD junction from genomic DNA. (C) Sanger sequencing confirmation of the integrated donor in the human genome. Sanger sequencing traces are aligned to the entire PCR of the LT-RD junction (top), with a zoomed-in version showing the nucleotides proximal to the LT-RD (bottom). Figure shows SEQ ID NOS: 798554, 798555, and 798556 in order of appearance.

FIGS. 42A-D shows engineering of the human genome by delivery of only a bridge editor. (A) Schematic depicting bridge editing of the human genome for inversions via delivery of only recombinase and bridgeRNA. A recombinase and bridgeRNA specific for a genomic donor and genomic target sequence results in inversion when the donor and target are on opposite strands. PCR of the RD-LT junction with primers L and L′ and the LD-RT junction with primers R and R′ detect recombination. Various orientations of target and donor result in inversion—one is shown here. (B-C) PCR detection of RD-LT and LD-RT for four different bridgeRNAs via agarose gel. The chromosomal locus targeted by the bridgeRNA is shown (above) as well as the relative orientation of the donor and target before and after recombination (bottom). (D) Example of Sanger sequencing confirmation of an inverted locus from panel b. Sanger sequencing traces are aligned to the entire PCR of the RD-LT junction (top left), with a zoomed in version showing the nucleotides proximal to the RD-LT (bottom left). Sanger sequencing traces are aligned to the entire PCR of the LD-RT junction (top right), with a zoomed in version showing the nucleotides proximal to the LD-RT (bottom right). Figure shows SEQ ID NOS: 798557, 798557, 798558, 798559, and 798558 in order of appearance. (E) Schematic depicting bridge editing of the human genome for excisions via delivery of only recombinase and bridgeRNA. A recombinase and bridgeRNA specific for a genomic donor and genomic target sequence results in excision when the donor and target are on the same strands. PCR of the LD-RT junction with primers G and G′ detect excision from the locus while PCR of the LT-RD junction with E and E′ detect the excised DNA. Various orientations of target and donor result in inversion—one is shown here.

FIGS. 43A-F show engineering of a split bridgeRNA system for recombination. (A) Schematic depicting recombination assay using an LE encoded bridgeRNA specific for a donor and target. Donor is SEQ ID NO: 795177. (B) Schematic depicting recombination assay using an LE encoded bridgeRNA and a separately expressed target binding loop (TBL). The target binding loop of the LE encoded bridgeRNA has been inactivated by reprogramming the LTG and RTG to have no complementarity to any sequence in the plasmids or organism, while the donor binding loop (DBL) is specific for the donor site sequence. (C) Comparison of recombination efficiency using the structure of the WT bridgeRNA (A) and the split bridgeRNA as depicted in (B). (D) Schematic depicting various split bridgeRNA systems. The bridgeRNA is depicted as a sequence where one or the other binding loop has been reprogrammed to have no specificity for a sequence found in the system. Some versions completely split the bridgeRNA into two, with a separate TBL and DBL. Some versions exclude additional accessory or unstructured nucleotides. The right hand panel indicates what the approximate specificity and structures may be for various iterations of a split bridgeRNA system. (E) Insertion of cargo into the lacZ gene of the E. coli genome. Insertion is performed using a bridgeRNA with the TBL targeted to the lacZ gene. Blue/white screening of lacZ activity using beta-galactosidase is used to pick colonies bearing the genomic insertion. PCR and agarose gel (right) confirms integration of the cargo into the genome. (F) Insertion of cargo into the lacZ gene of the E. coli genome. Insertion is performed using a bridgeRNA with the TBL reprogrammed to have no specificity for a sequence in the plasmid system or E. coli, while a separate TBL specific for the lacZ gene is expressed from a synthetic promoter such as the system depicted in (B). Blue/white screening of lacZ activity using beta-galactosidase is used to pick colonies bearing the genomic insertion. PCR and agarose gel (right) confirms integration of the cargo into the genome.

FIGS. 44A-B show a summary of mismatch tolerance between an IS110 bridgeRNA target binding loop and its target. (A) Schematic depicting antibiotic resistance reporter design. A minimal donor (22 bp) is encoded on a plasmid adjacent to a kanamycin resistance gene. A second plasmid encodes the target, bridgeRNA, and transposase. The target is linked to the bridgeRNA using a barcode. Recombination between the donor and target plasmid results in E. coli survival, and functional bridgeRNA target loop and target pairs are recorded using next generation sequencing (left). Schematic depicting target specificity screen design. The target and target loop are varied, except for the core of the target and the subsequences of the LTG and RTG that bind the core. The donor loop (and donor) are held constant. Target and target loop pairs are designed to assay single mismatches, double mismatches, and total mismatches. Targets in the screen are selected to reduce the number of off-targets in the E. coli genome. (B) Sequence Abundance of target and target loop pairs. Abundance is measured by barcode counts per million reads. Target/target loop pairs with zero mismatches are generally more abundant, while increasing the number of mismatches decreases abundance. (D) Sequence logo of top quintile of targets. The relative enrichment of nucleotides at each position of the target are shown for target/target loop pairs with zero mismatches in the top quintile of 6364 target/target loop pairs. (B) Mismatch tolerance at each position of the 11 bp target sequence. The x-axis shows the target position, with the CT core held constant. The top panel shows the target nucleotide recovery frequency when the target binding loop contains an A at each guide position as a percentage of recovered recombinants at each position. The second, third, and fourth panel shows the same but when the target-binding loop contains a C, G, or U at each position. Target positions are depicted as the top strand of the DNA.

### DETAILED DESCRIPTION

The present invention relates to the IS110 transposon family. The IS110 transposons encode both a “bridgeRNA” molecule and a transposase protein. The bridgeRNA molecule in concert with the transposase mediates site-specific recombination between one or more DNA molecules containing a target site sequence and a donor site sequence. The target site sequence and the donor site sequences can be on the same DNA molecule or different DNA molecules. Generally, as used herein, the target site and donor site sequences are simply nucleic acid sequences that associate with, or are recognized by, an IS110 bridgeRNA and transposase complex, and depending on the orientation of these sequences and whether these sequences are on the same or different molecules, a transposition reaction will occur resulting in recombination between the target site and donor site sequences such that the result is either an insertion (or translocation), excisive recombination, or inversion. Thus, for insertion or translocation reactions, the target sequence and the donor sequence are on different molecules. For excisive recombination and inversion, the target and donor site sequences are on the same molecule, and depending on the orientation of the target and donor site sequences, intervening sequences are excised or inverted. Such recombination reactions may be employed to recombine any DNA sequence with any other DNA sequence in a programmable manner, without any requirements to use DNA sequences originating from the IS110 element. More specifically, the present invention provides recombinant IS110 transposons where the encoded bridgeRNA molecule is programmable by modifying sequences in the target and/or donor binding loops of the bridgeRNA thereby engineering the bridgeRNA to specifically bind sequences of interest. In one category of programmable transposition, the bridgeRNA is designed such that a donor DNA molecule of interest can be recombined with a target DNA molecule of interest to effectuate insertion of a sequence located on a different DNA molecule or translocation of sequences on different DNA molecules. In another category of programmable transposition, the bridgeRNA is designed such that a donor DNA sequence of interest can be recombined with a target DNA of interest to effectuate excision or inversion of intervening sequences located on the same DNA molecule. Further, the invention also encompasses non-programmed uses of the IS110 family of transposons. For example, a non-programmed IS110 bridgeRNA (target and donor binding loops are not modified to change the binding specificity of the bridgeRNA) and transposase complex can be used to target naturally occurring target and donor site sequences in prokaryotic genomes, naturally occurring target and donor site sequences in eukaryotic genomes, introduced target and donor site sequences in prokaryotic genomes, and introduced target and donor site sequences in eukaryotic genomes.

### A. Definitions

The terms “polynucleotide”, “nucleotide sequence”, “nucleic acid”, “nucleic acid molecule”, “nucleic acid segment”, and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “DNA” refers to, without limitation, deoxyribonucleic acid, which includes, but is not limited to genomic or non-genomic DNA that exists within a cell or the isolated form of such DNA. Genomic or non-genomic DNA includes without limitation, chromosomal or non-chromosomal DNA such as episomal, viral, plasmid, mitochondrial, cellular, or chloroplast DNA.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “nuclease” refers to an agent capable of breaking a phosphodiester bond linking a nucleotide residue in a nucleic acid molecule, such as a protein or small molecule. In some embodiments, the nuclease is an enzyme capable of binding a nucleic acid molecule, and breaking a phosphodiester bond that links a nucleotide residue within the nucleic acid molecule. The nuclease may be an endonuclease that cleaves the phosphodiester bond within the polynucleotide chain or an exonuclease that cleaves the phosphodiester bond at the end of the polynucleotide chain. In some embodiments, the nuclease is a site-specific nuclease that binds to and/or cleaves a particular phosphodiester bond within a particular nucleotide sequence. The term “nickase” refers to an endonuclease which cleaves only a single strand of a DNA duplex.

As used herein, the term “excisionase” refers to a host-derived, bacteriophage, or mobile genetic element sequence-specific DNA binding protein. It is involved in removing DNA from nucleotide sequences, repairing the DNA with or without a sequence scar. The removed DNA may be in the form of linear or circular ssDNA or dsDNA.

“Sequence-specific” refers to, but is not limited to, recombination or a recombination event which occurs at a predictable locus or identifiable nucleotide sequence or modification of a nucleotide at a predetermined sequence location.

The term “transposon”, as used herein, refers to a polynucleotide (or nucleic acid segment), which can be copied or moved to a new nucleic acid sequence context through the action of a transposase. An insertion sequence (IS) element refers to a transposon that encodes the minimal components necessary for recombination of a nucleotide sequence (e.g. a transposase and a bridgeRNA). An IS element may be referred to herein as an IS element, IS110 element, or transposon.

The term “transposase” as used herein refers to an enzyme, which is a component of a functional nucleic acid-protein complex (e.g., a transpososome) capable of transposition and which mediates transposition. The transposase may comprise a single protein or comprise multiple proteins. A transposase may be an enzyme capable of forming a functional complex with a transposon end, transposon end sequences, or transposon-derived sequences. The term “transposase” may also refer in certain embodiments to integrases, recombinases, invertases, or excisionases. Described herein are transposases derived from the IS110 family of transposons. The IS110 “transposases” described herein are also referred to as IS110 “recombinases.”

The expression “transposition reaction” used herein refers to a reaction wherein a transposase recombines a DNA polynucleotide comprising a donor site sequence with a DNA polynucleotide comprising a target site sequence, e.g., an integration reaction, a recombination reaction, an inversion reaction, or an excision reaction. A transposition reaction may occur when the donor site sequence and target site sequence are on one or more DNA molecules. Both the target and donor site sequences may contain a sequence or secondary structure. The target site and the donor site sequences may contain a sequence or secondary structure recognized by the transposase and/or an insertion motif sequence where the transposase cuts or creates staggered breaks in the target polynucleotide sequence.

The term “transposon end sequence” as used herein refers to the nucleotide sequences at the distal ends of a transposon. The transposon end sequences, or subsequences thereof, may be the DNA sequences recognized by the transposase to form a transpososome complex and to perform a transposition reaction. In certain embodiments described herein, the transposon end sequences are derived from the non-coding end sequences of the IS110 family of transposons.

The practice of aspects of the present invention can employ, unless otherwise indicated, conventional techniques of cell biology, cell culture, molecular biology, transgenic biology, microbiology, recombinant DNA, and biochemistry, which are within the skill of the art. Such techniques are explained fully in the literature. See, e.g., Molecular Cloning A Laboratory Manual, 3rd Ed., ed. by Sambrook (2001), Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture Of Animal Cells (R. I. Freshney, Alan R. Liss, Inc., 1987); Immobilized Cells and Enzymes (TRL Press, 1986); B. Perbal, A Practical Guide To Molecular Cloning (1984); the series, Methods In Enzymology (Academic Press, Inc., N.Y.), specifically, Methods In Enzymology, Vols. 154 and 155 (Wu et al. eds.); Gene Transfer Vectors For Mammalian Cells (J. H. Miller and M. P. Calos eds., 1987, Cold Spring Harbor Laboratory); Immunochemical Methods In Cell And Molecular Biology (Caner and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986) and subsequent versions thereof.

One skilled in the art can obtain a protein in several ways, which include, but are not limited to, isolating the protein via biochemical means or expressing a nucleotide sequence encoding the protein of interest by genetic engineering methods.

A protein is encoded by a nucleic acid (including, for example, genomic DNA, messenger RNA (mRNA), complementary DNA (cDNA), synthetic DNA, as well as any form of corresponding RNA). Nucleic acids encoding a protein can be produced via recombinant DNA technology and such recombinant nucleic acids can be prepared by conventional techniques, including chemical synthesis, genetic engineering, enzymatic techniques, or a combination thereof.

### B. IS110 Elements

The IS110 family of transposons refer to a family of transposons that are widespread in prokaryotic genomes. They are categorized into two groups, the IS110 group and the IS1111 group, and they encode transposases that cumulatively demonstrate a range of insertion site specificities. The IS110 transposases can exhibit invertase and excisionase activity, in addition to their transposase activity.

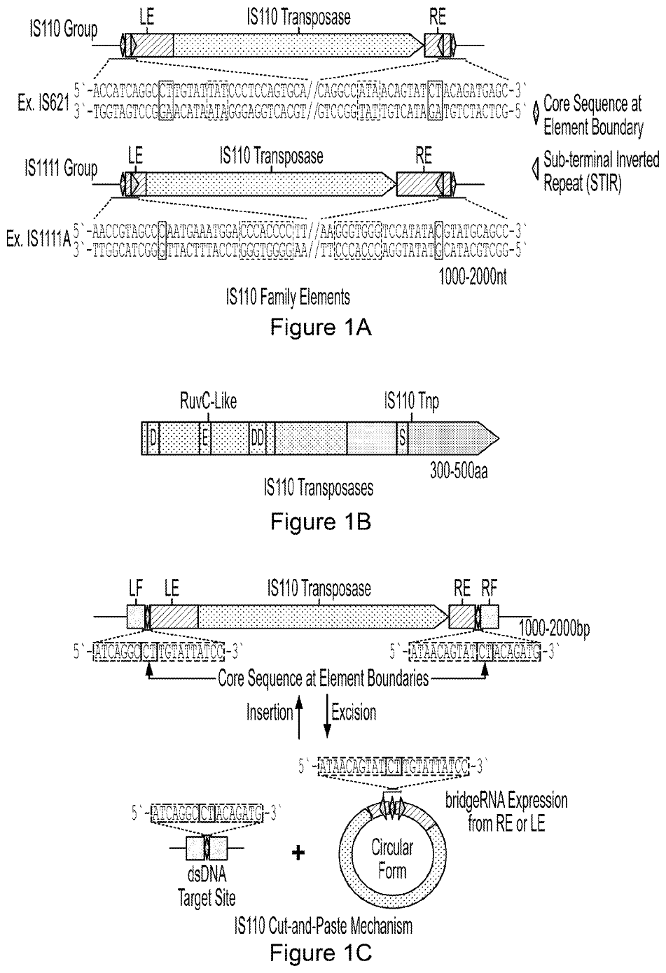

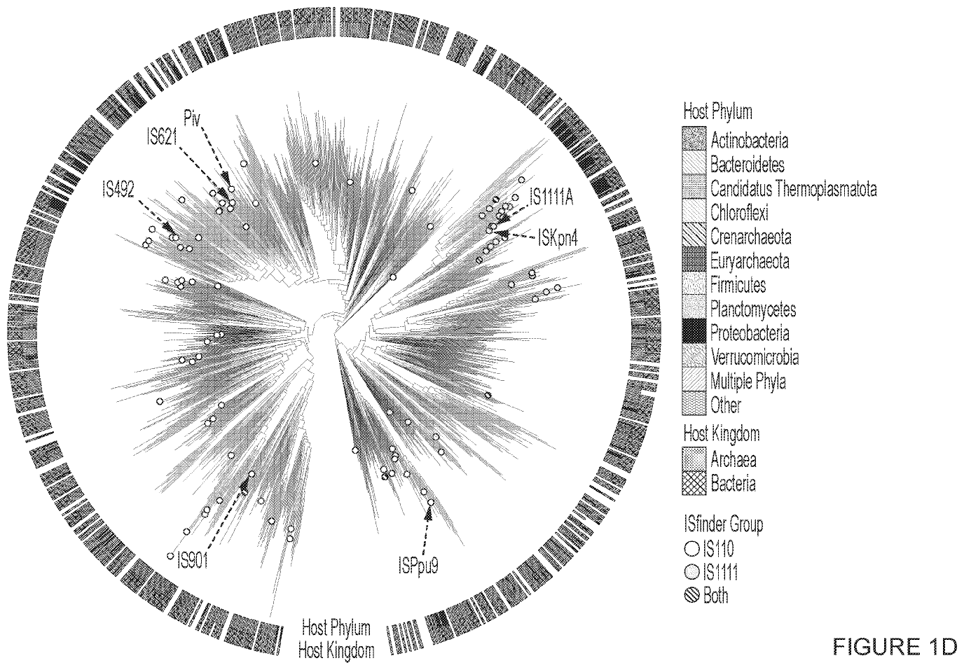

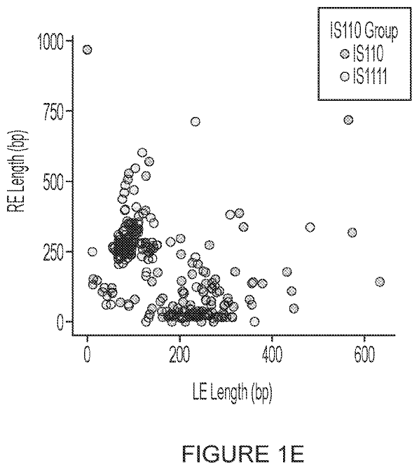

The life-cycle of an IS110 element is depicted in FIG. 1C. A linear IS110 element integrated into a target site comprises a left non-coding end (LE), a coding sequence for a transposase (Tpase) and a right non-coding end (RE), and, in some embodiments, the IS110 element is flanked by a repeated core sequence as shown in FIG. 1C. IS110 elements excise themselves, resulting in pre-insertion (“target”) site bearing LF-core (if present)-RF, and a circular element with RE-core (if present)-LE-Tpase. Concatenation of the RE-LE junction forms a “donor” site sequence as a subsequence of the RE-LE junction, which, if present, includes the other core sequence found on the integrated element. The donor site sequence may also include sub-terminal inverted repeats (STIR). Concatenation of the RE-LE may also form a promoter which, in the appropriate cellular context, may promote expression from the LE or RE of a RNA molecule referred to herein as bridgeRNA. The promoter may also promote expression of the transposase in the appropriate cellular context. The bridgeRNA encoded within the LE or RE forms an RNA-protein complex with the transposase and recognizes the donor site and/or the target site sequences to mediate transposition. The circular form of the element can reinsert into the target site or insert into any other target site sequence recognized by the bridgeRNA-transposase complex.

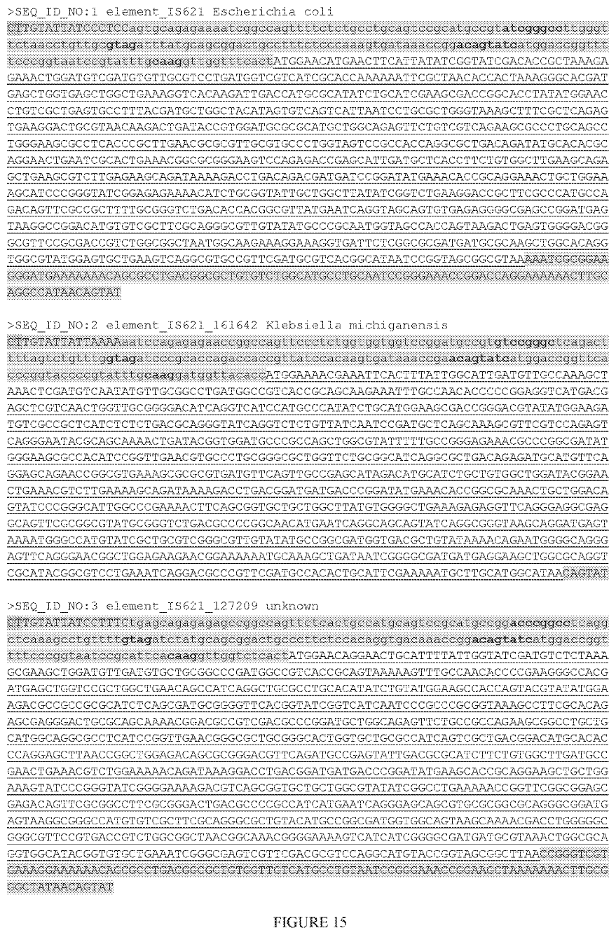

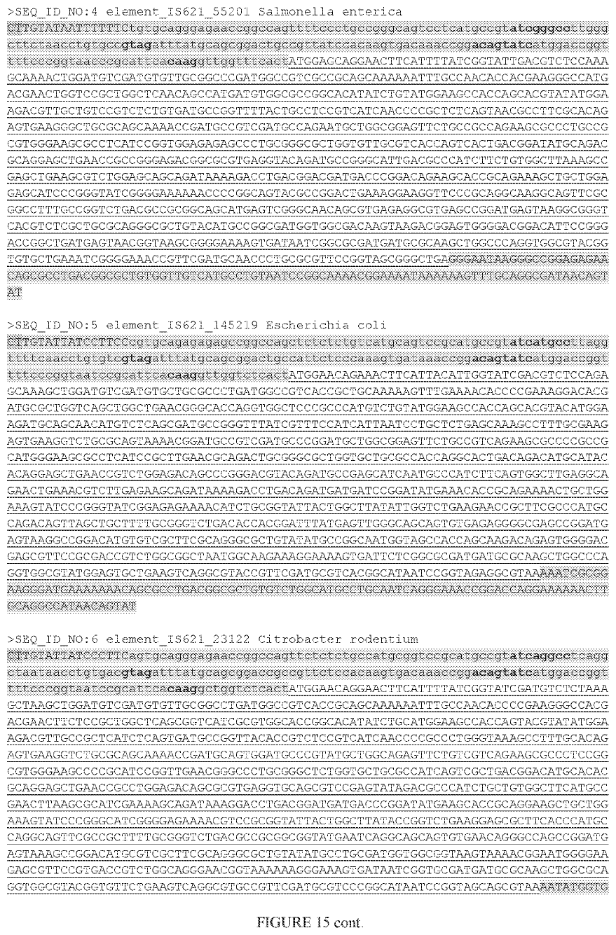

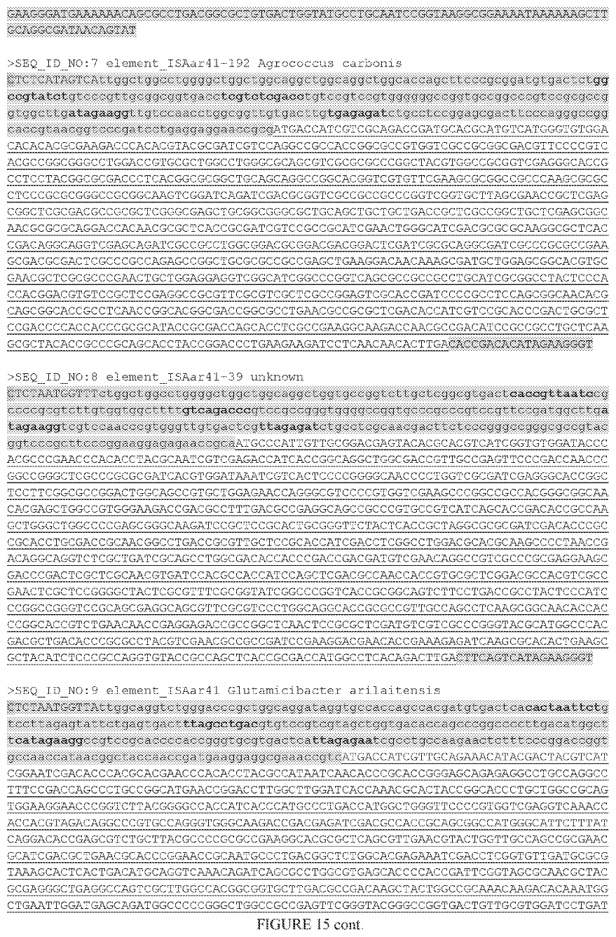

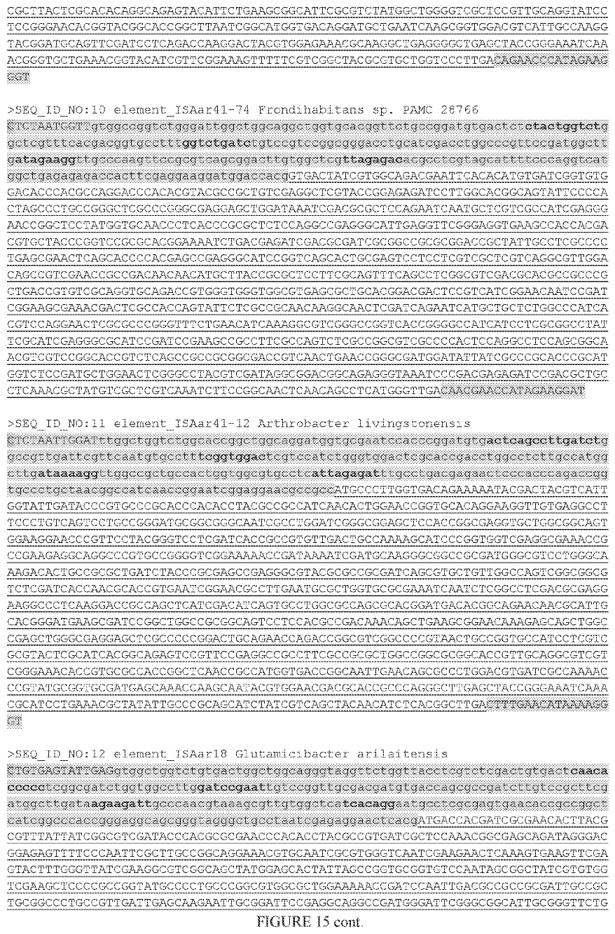

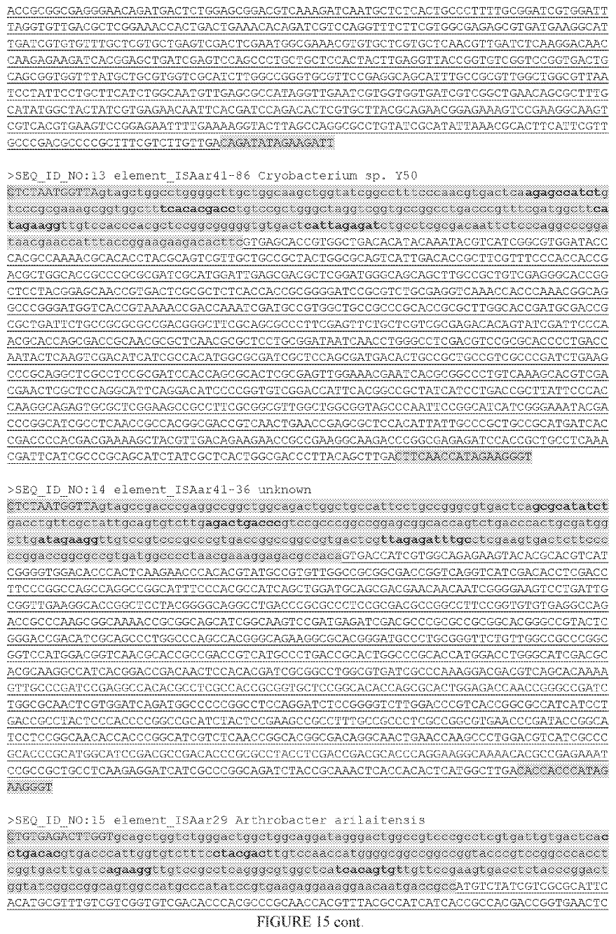

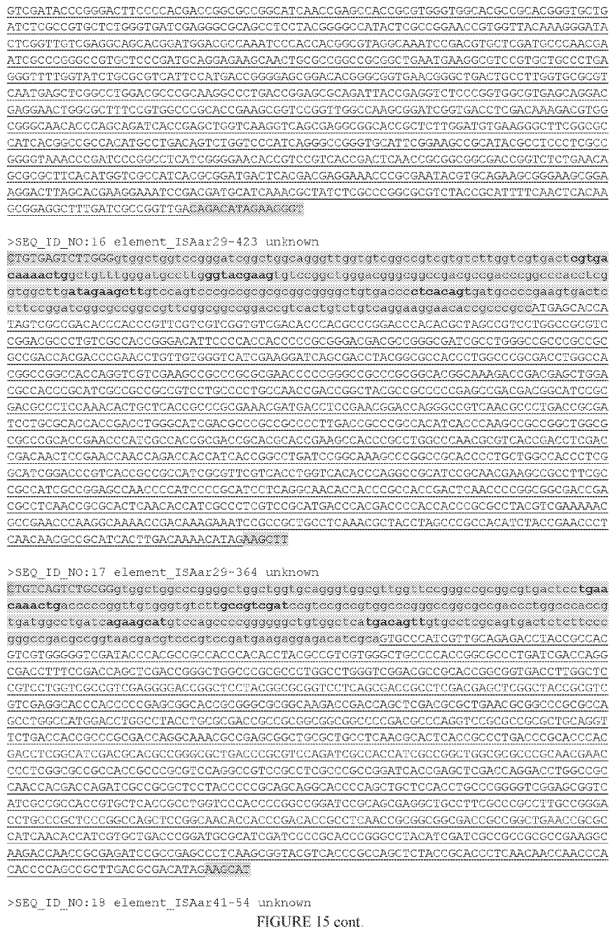

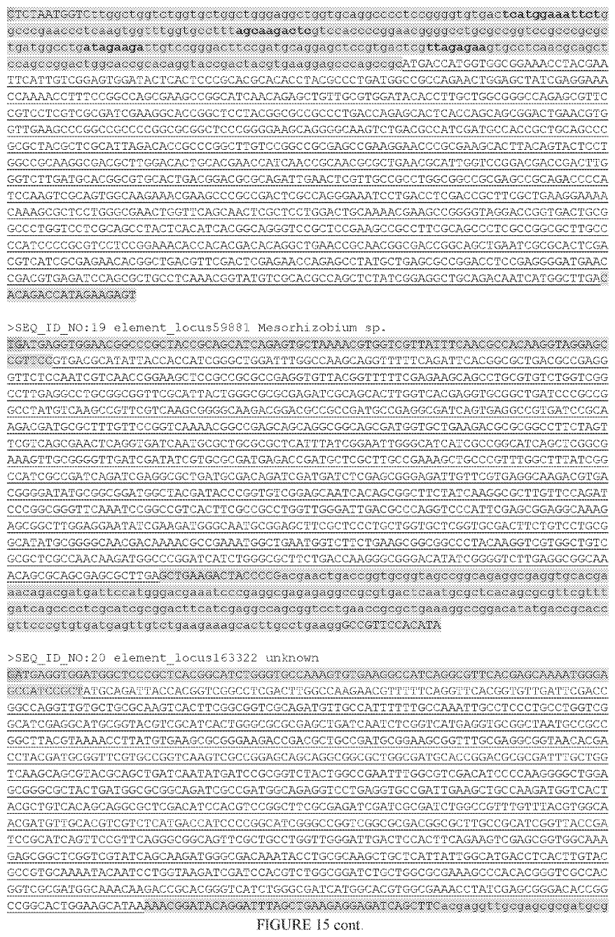

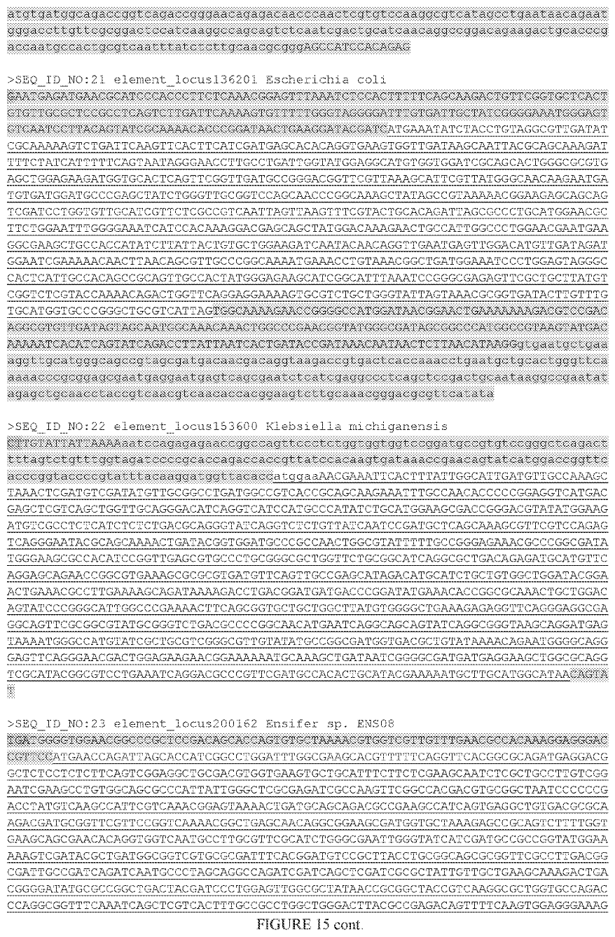

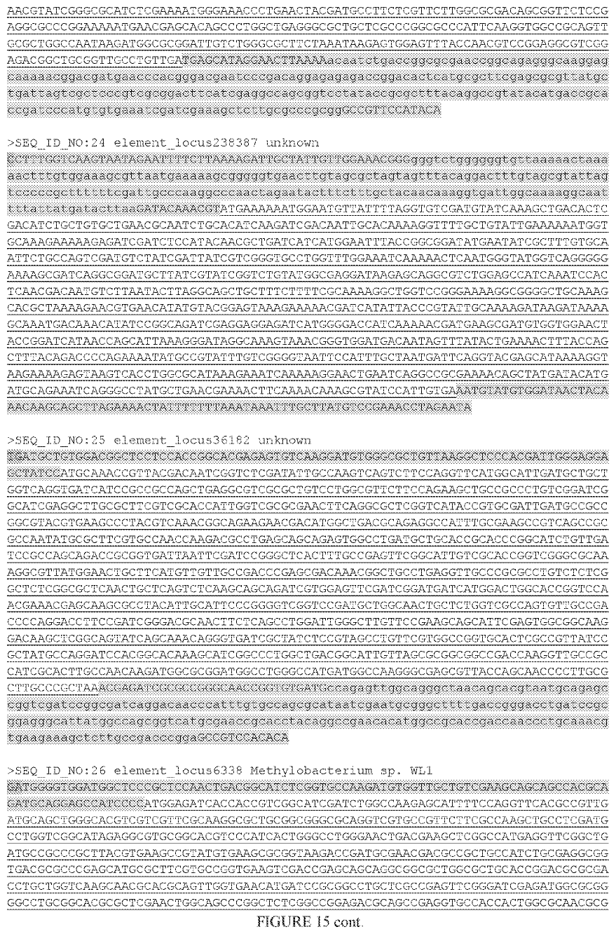

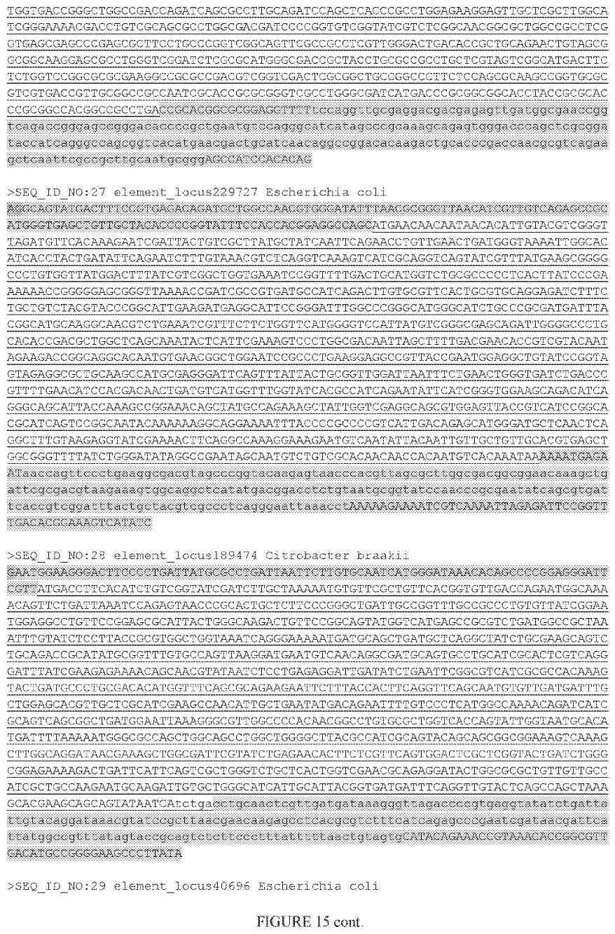

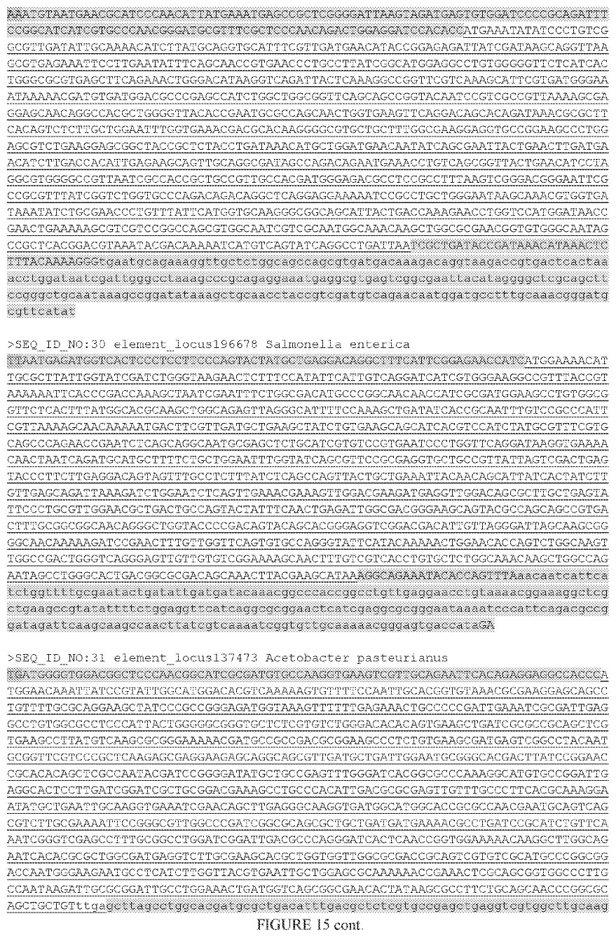

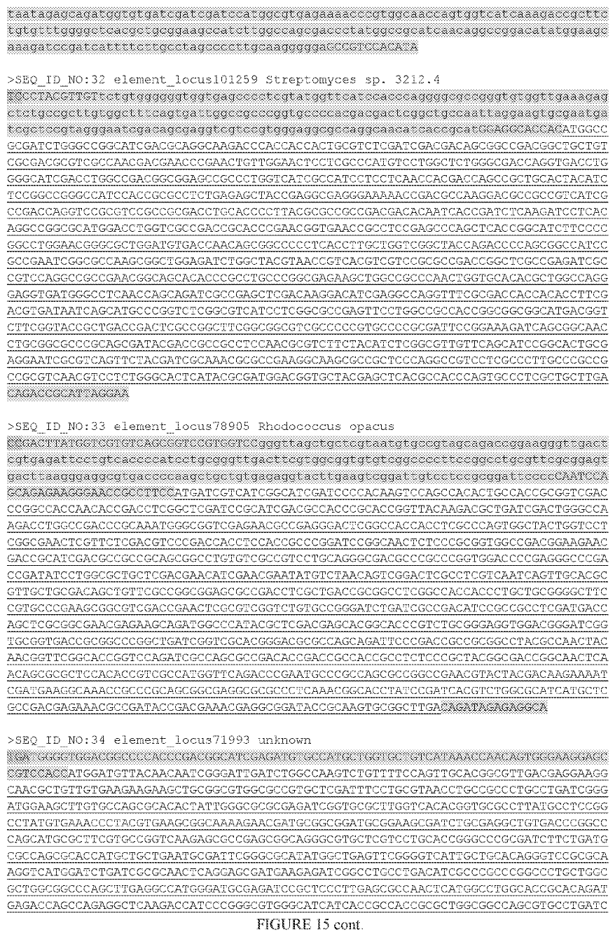

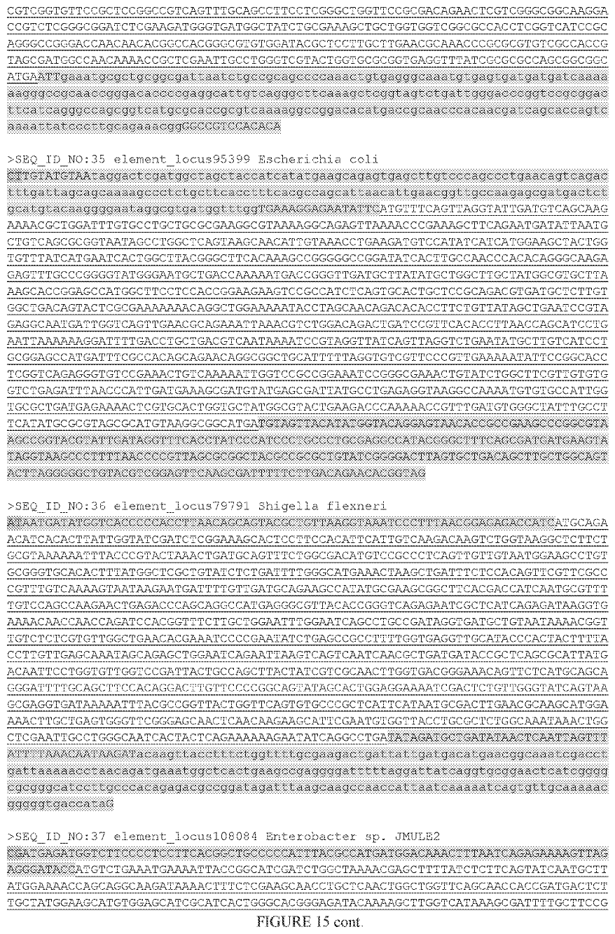

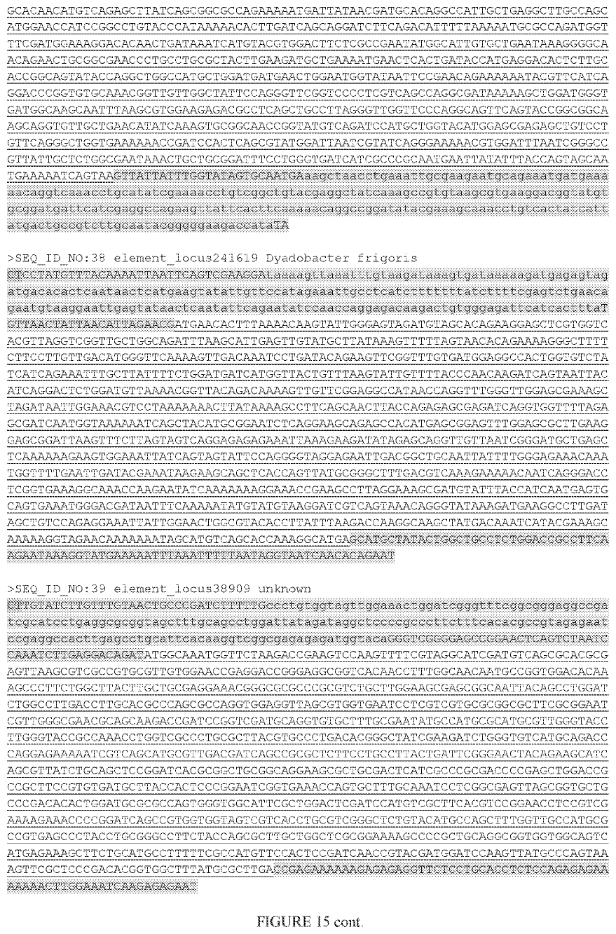

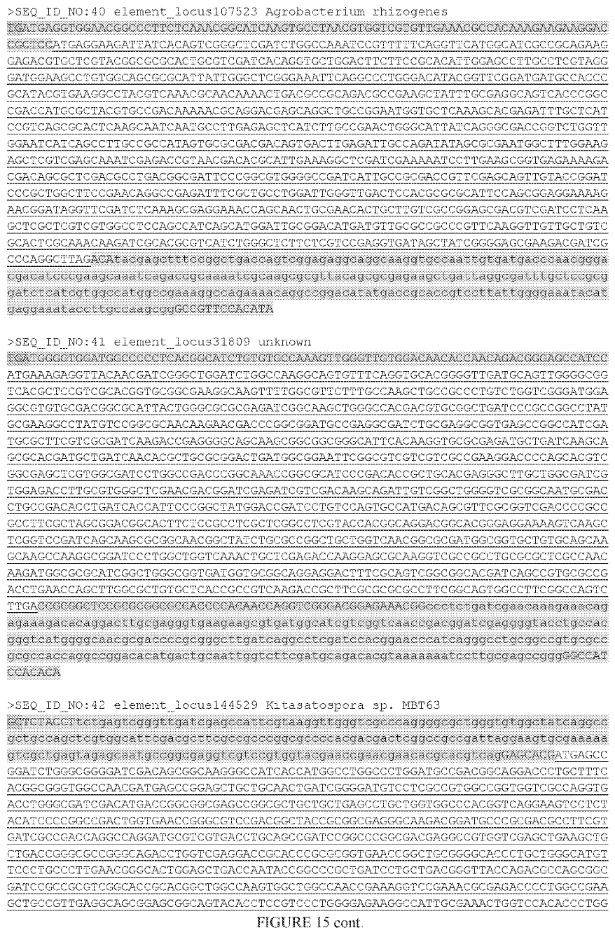

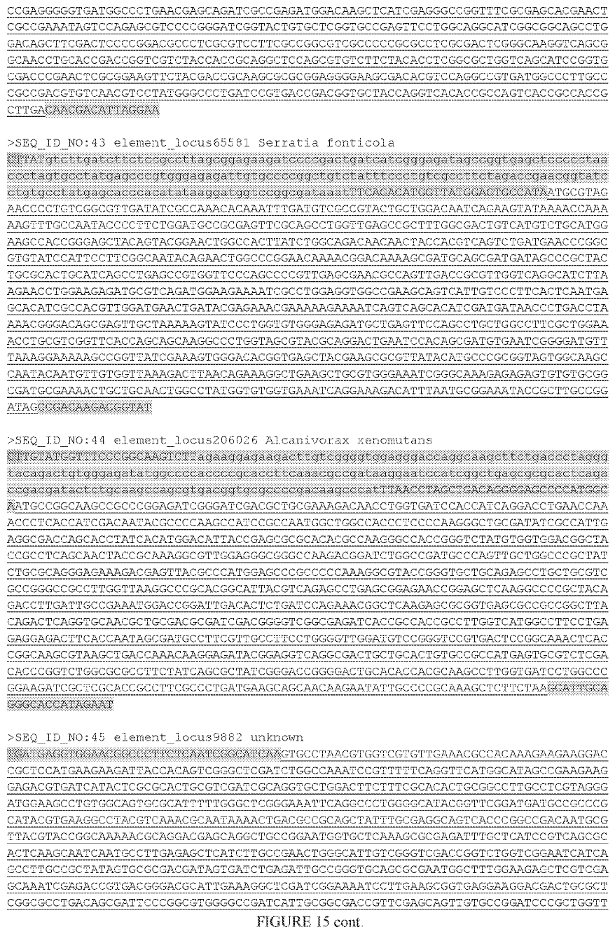

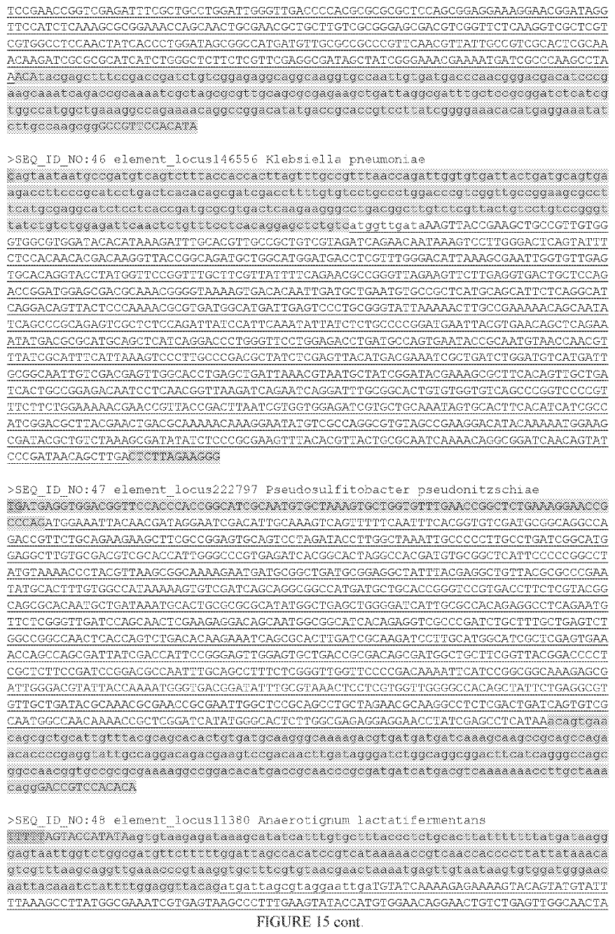

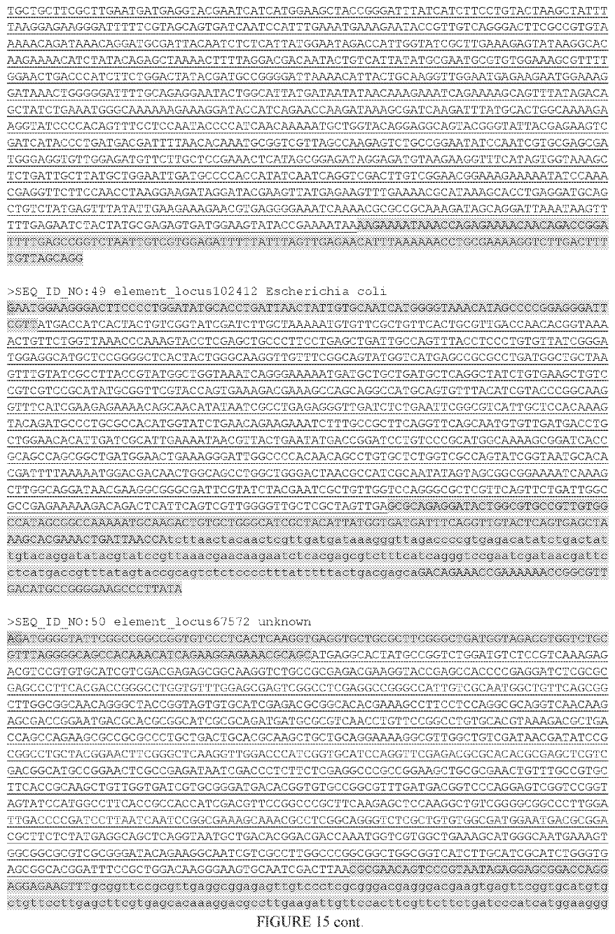

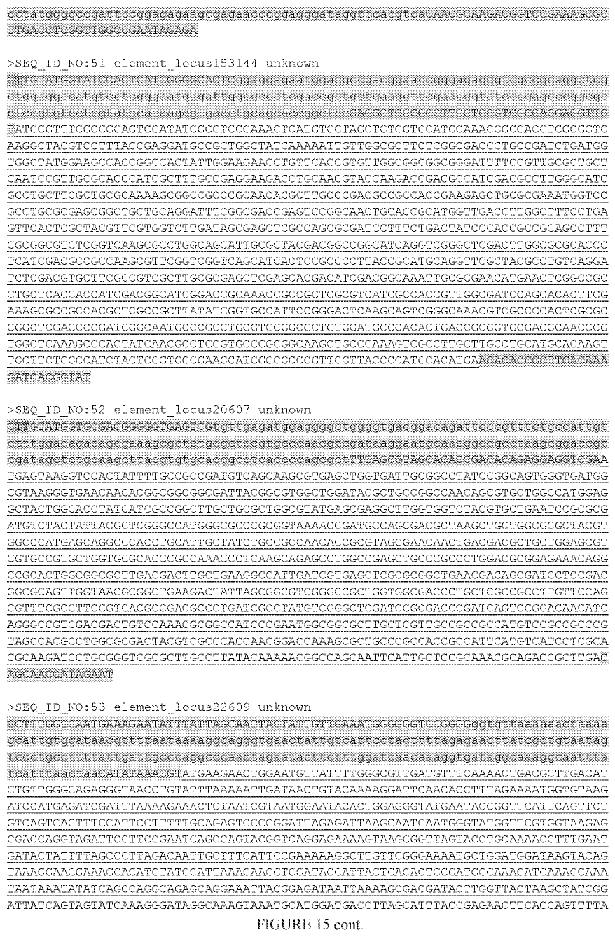

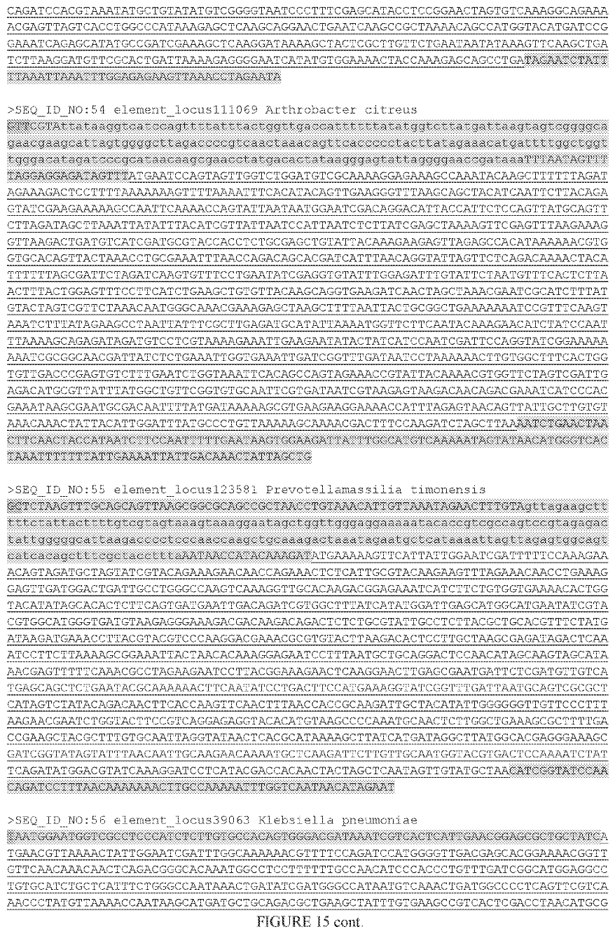

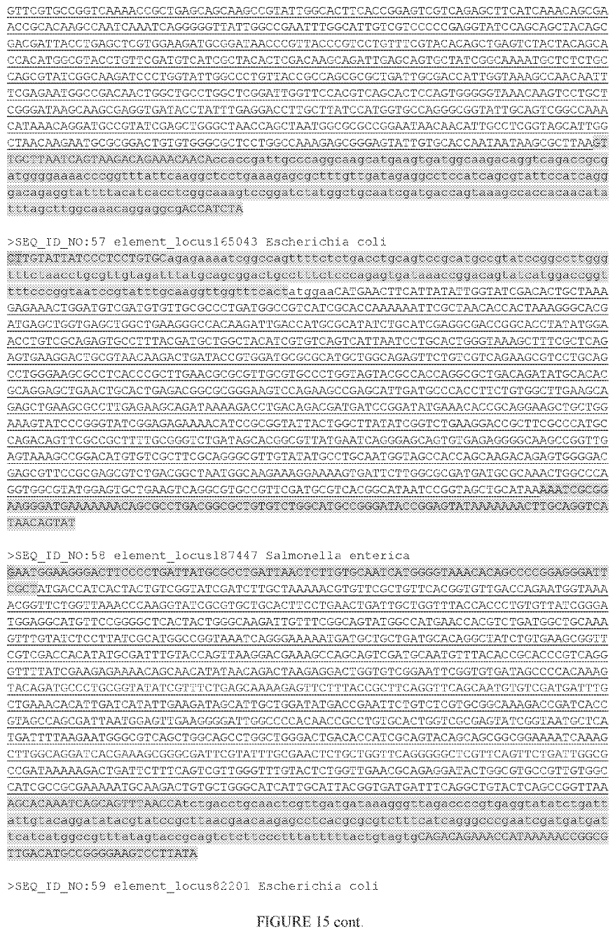

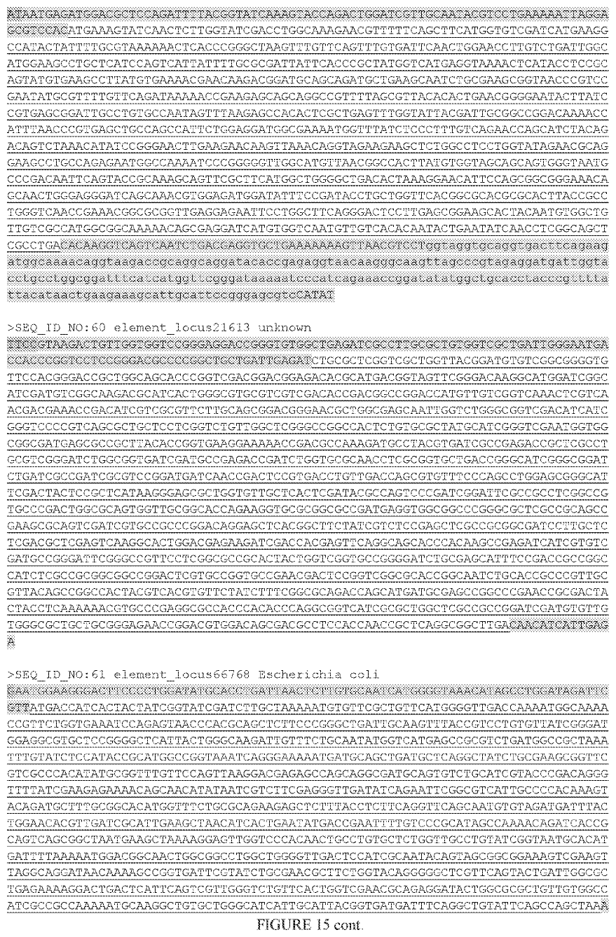

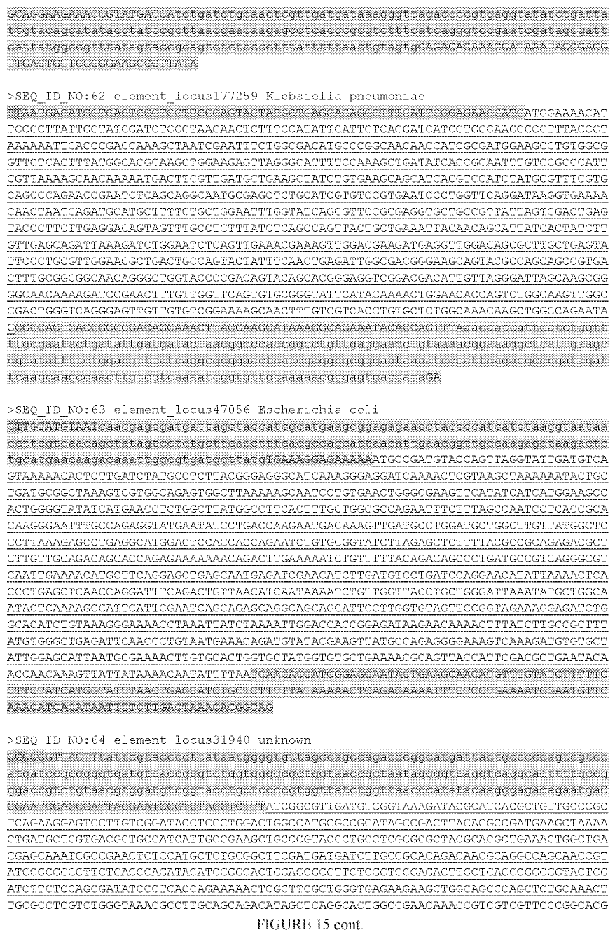

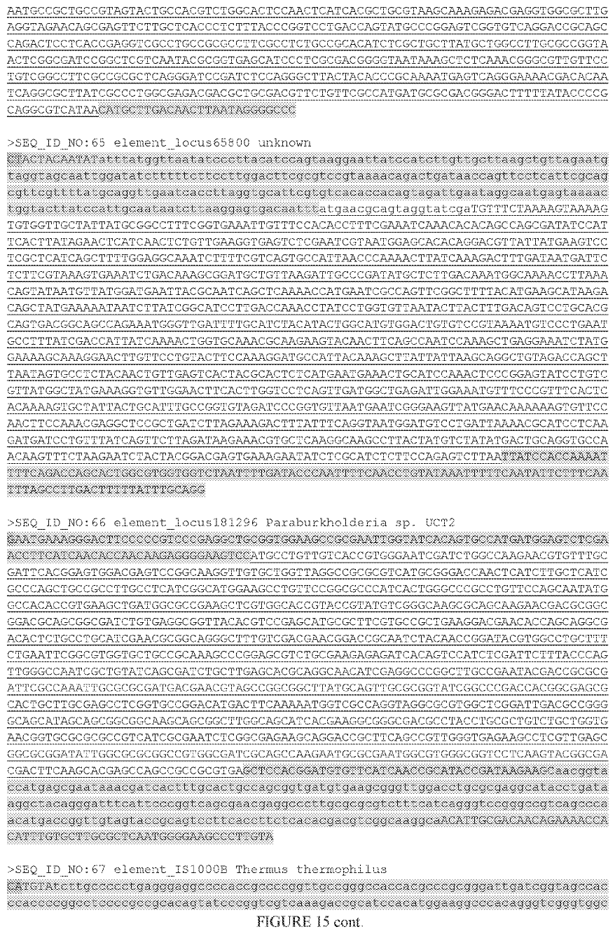

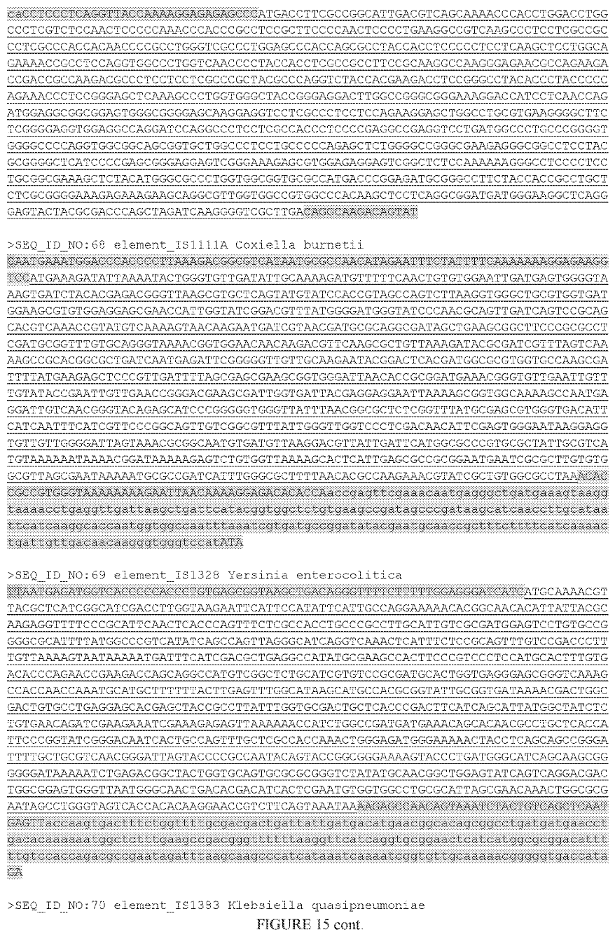

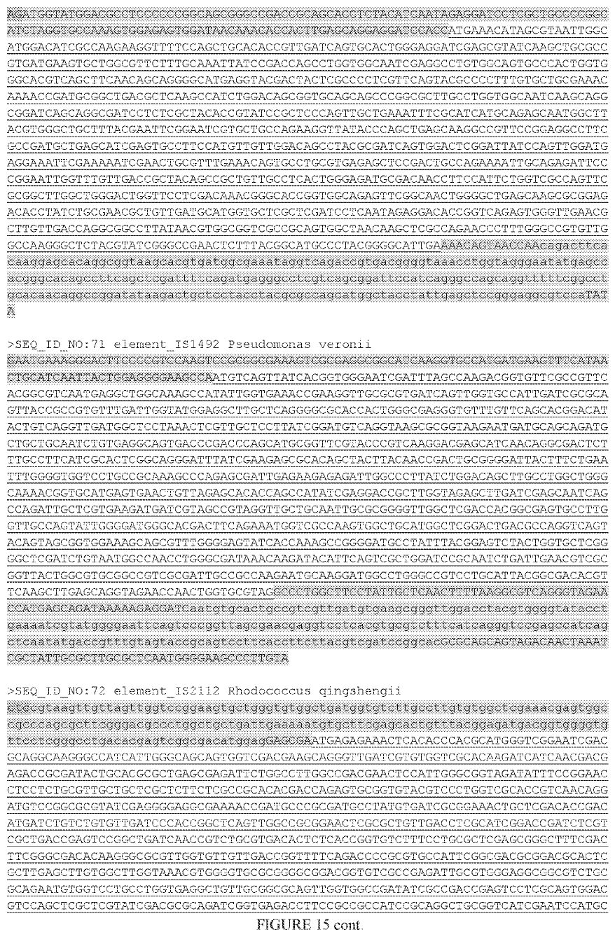

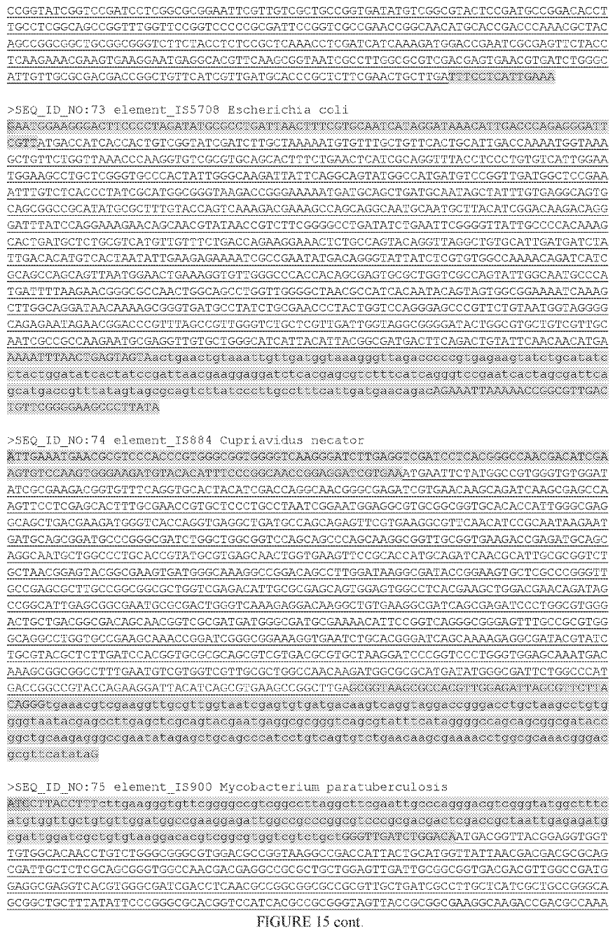

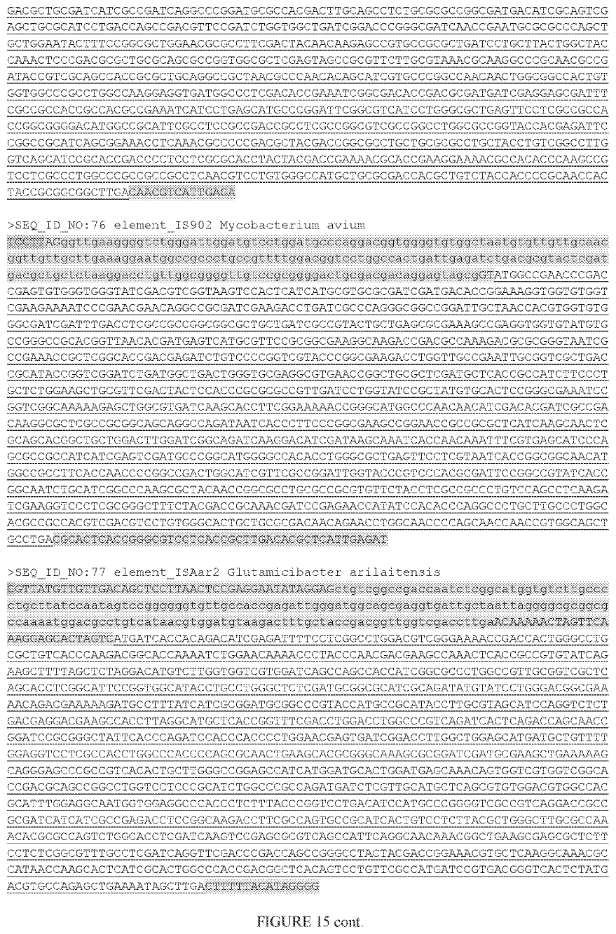

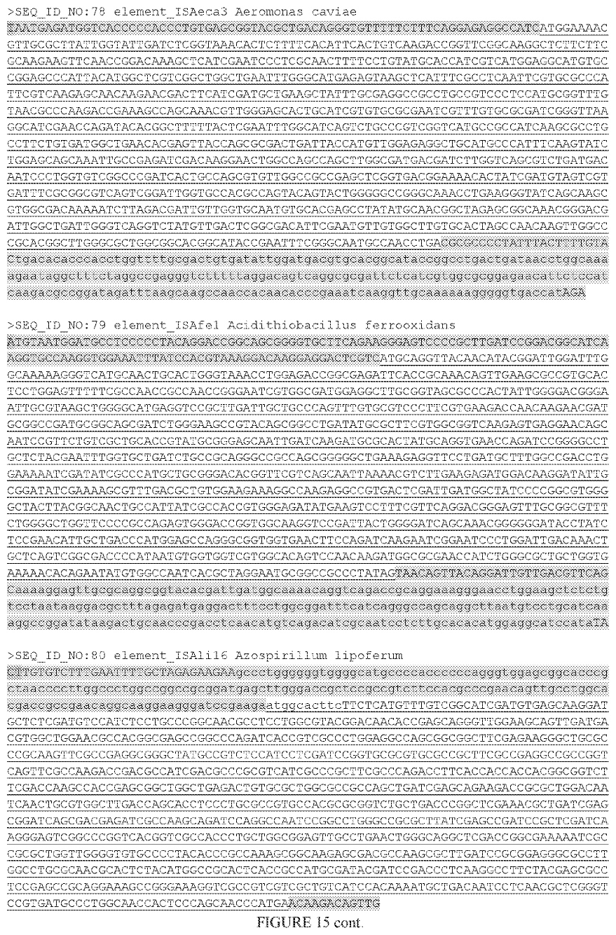

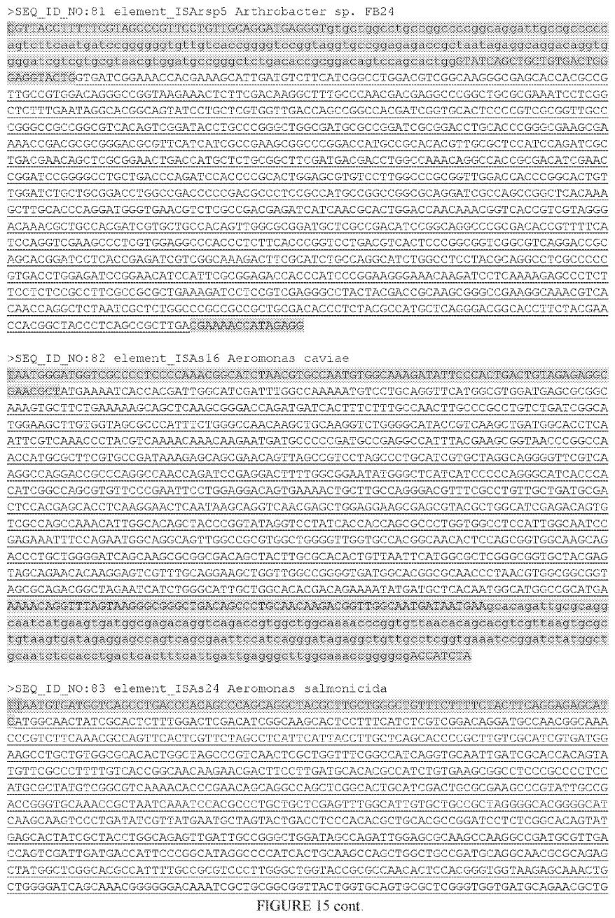

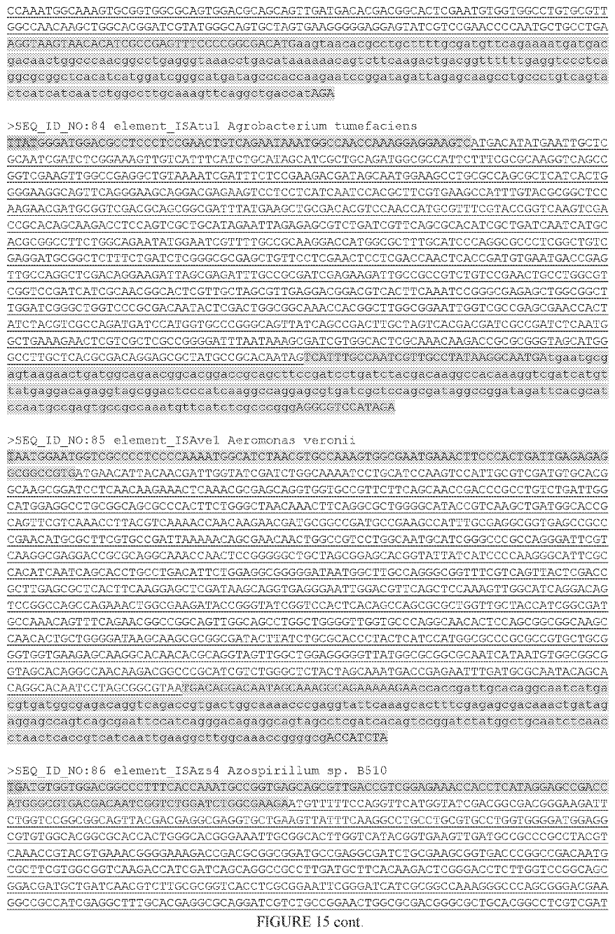

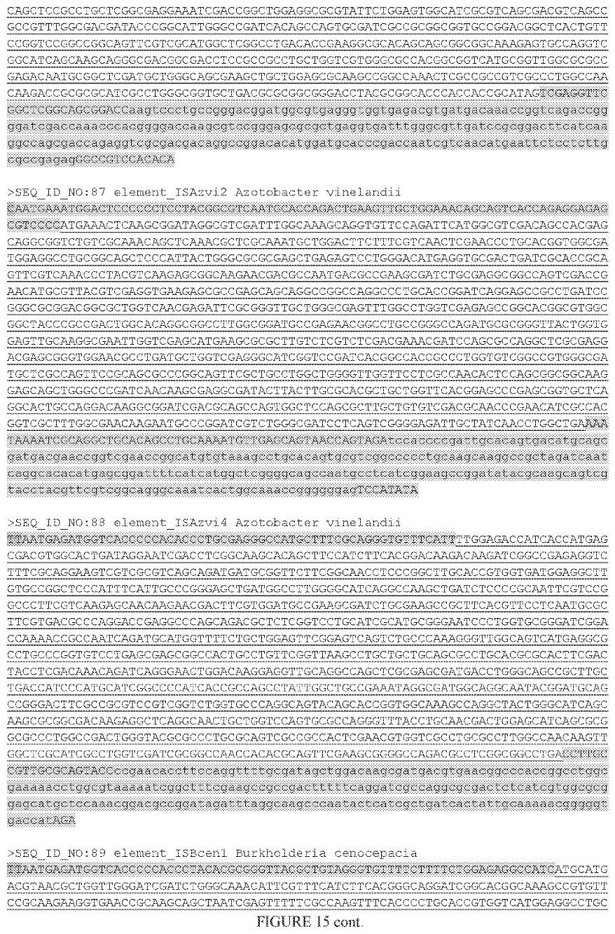

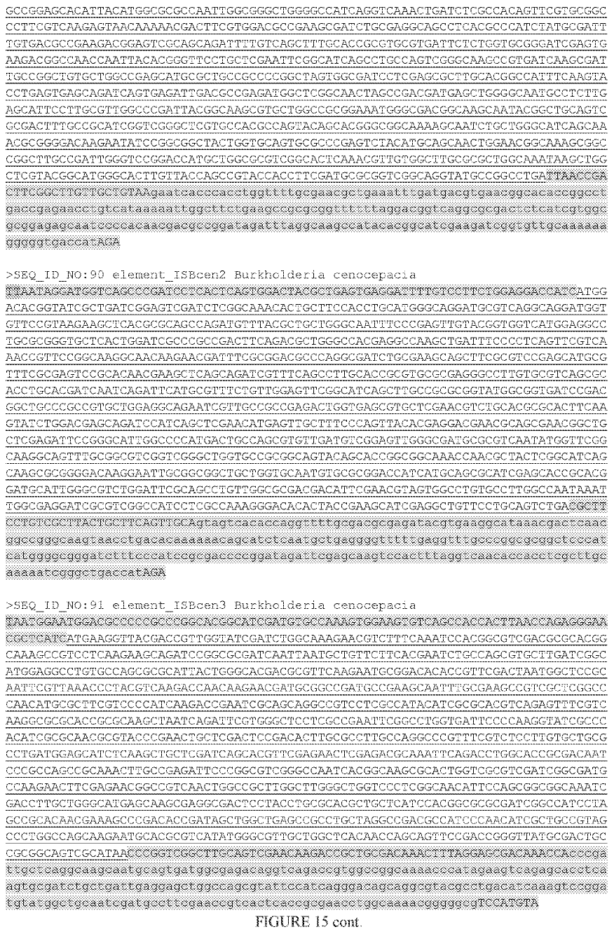

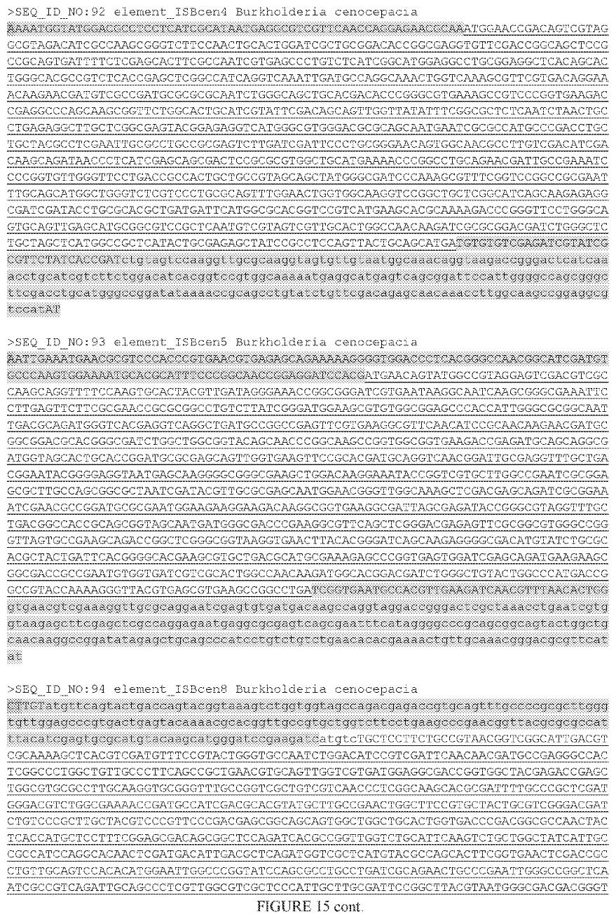

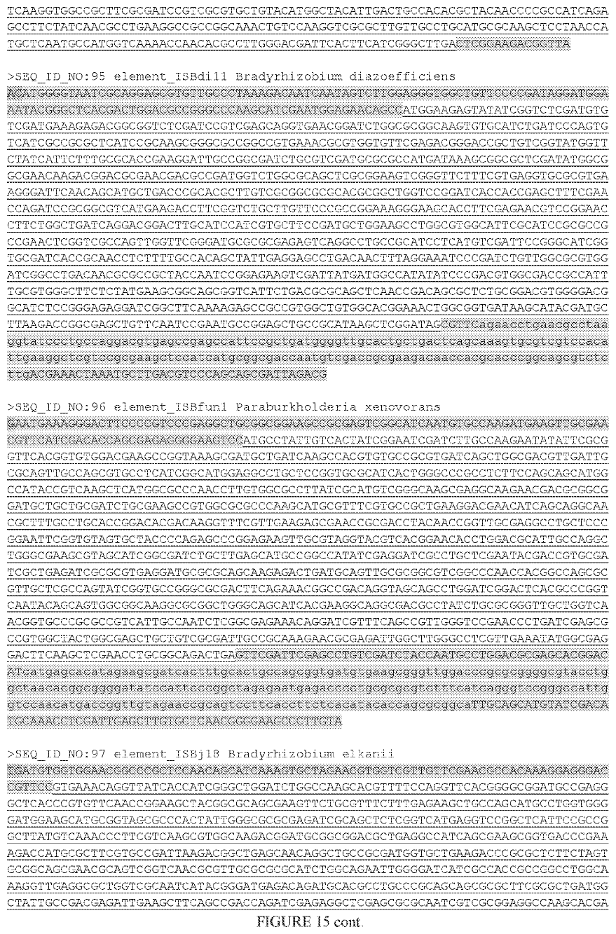

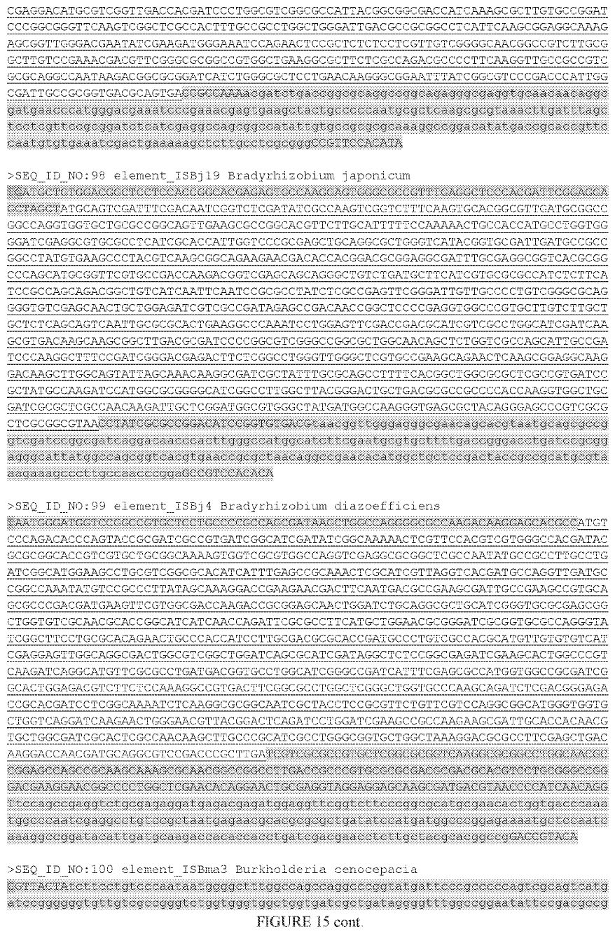

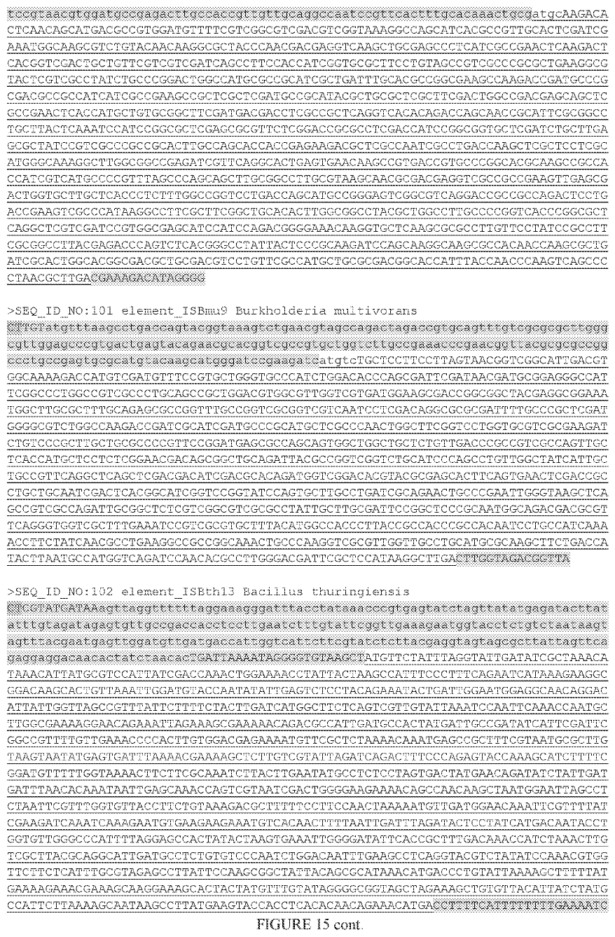

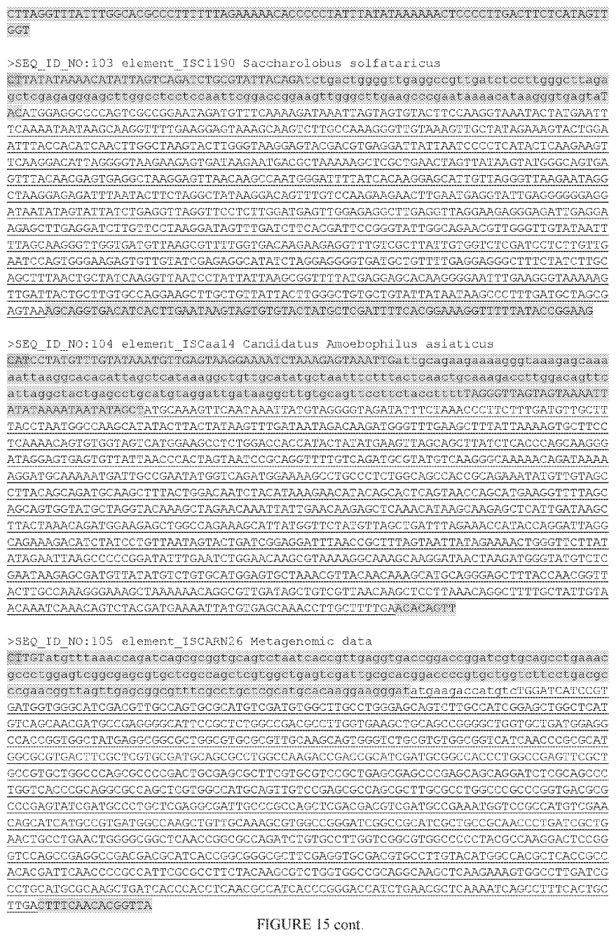

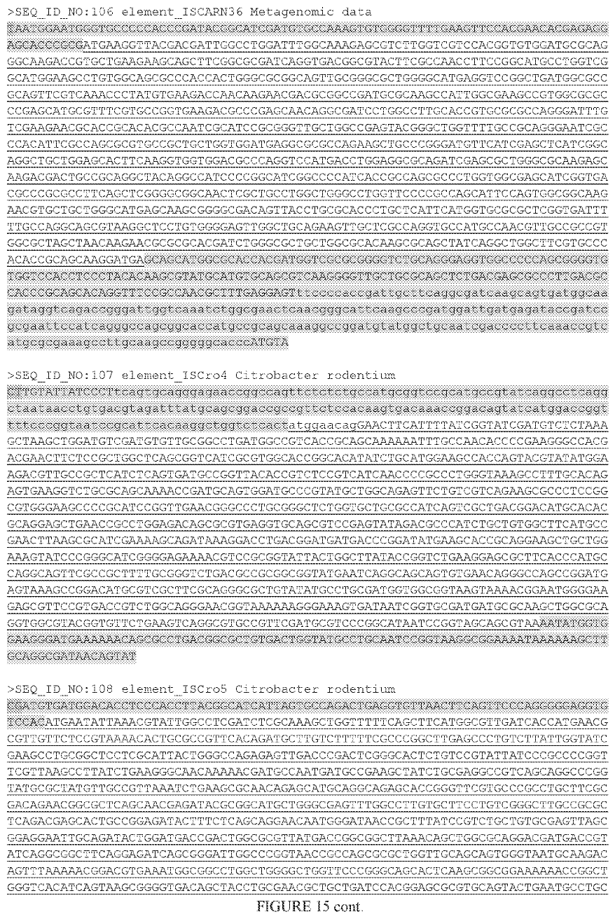

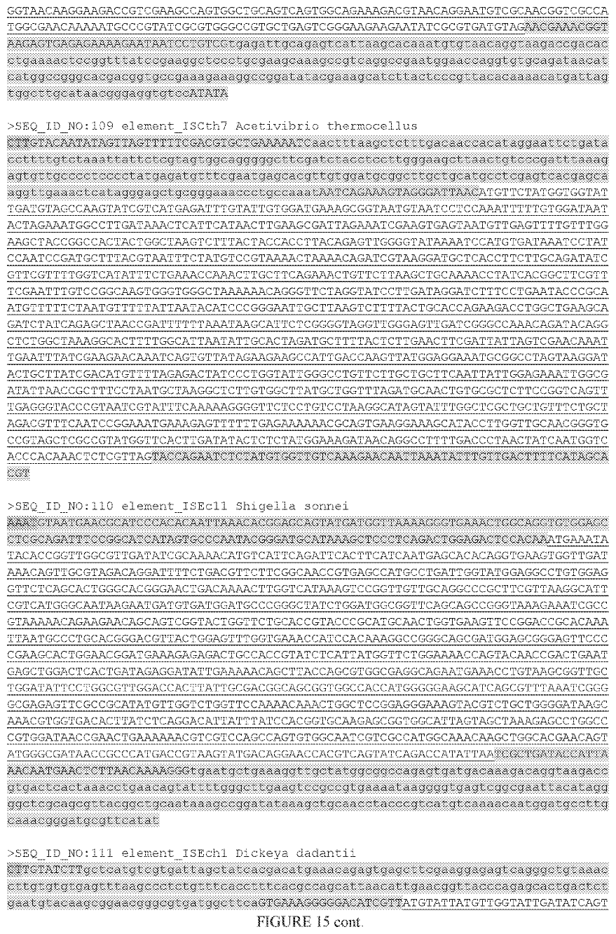

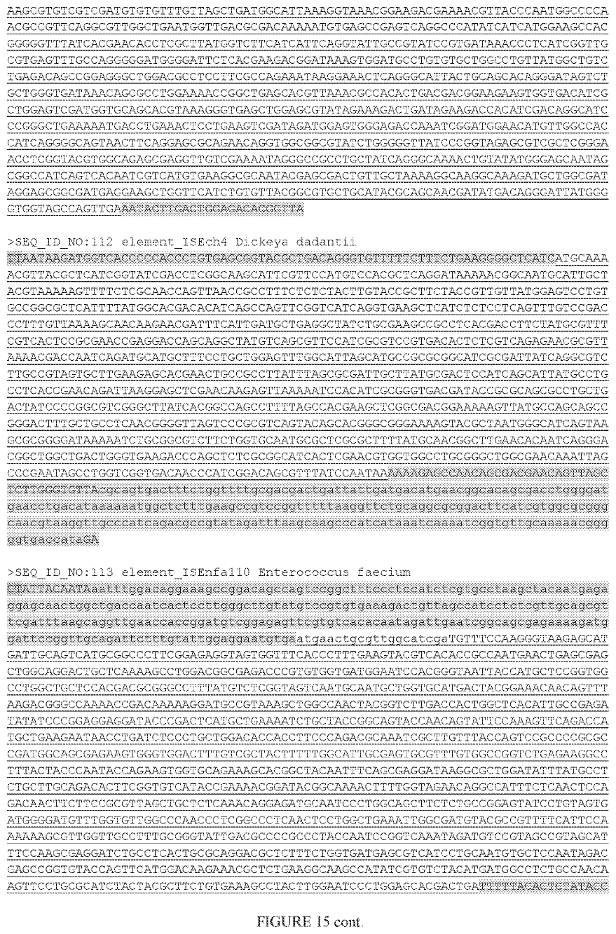

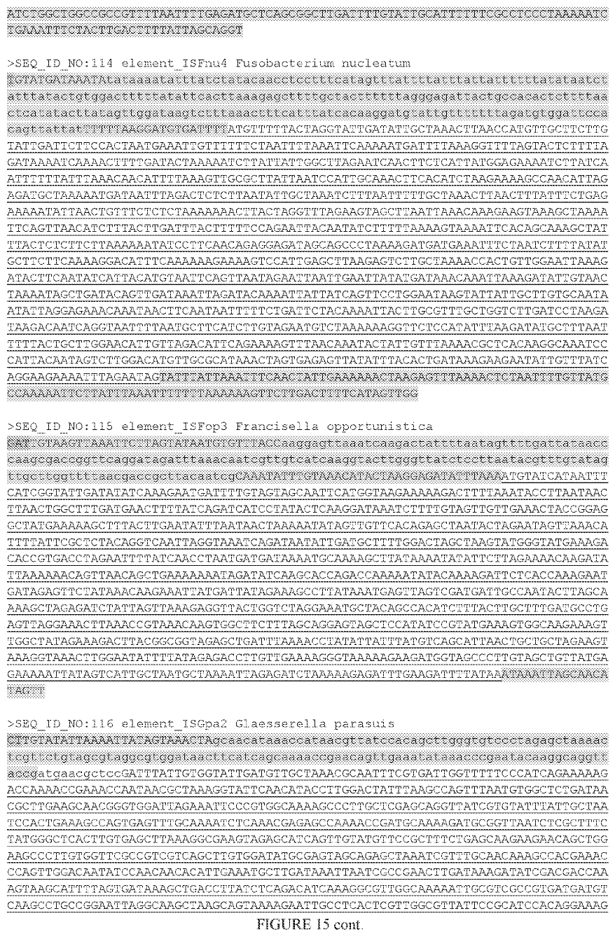

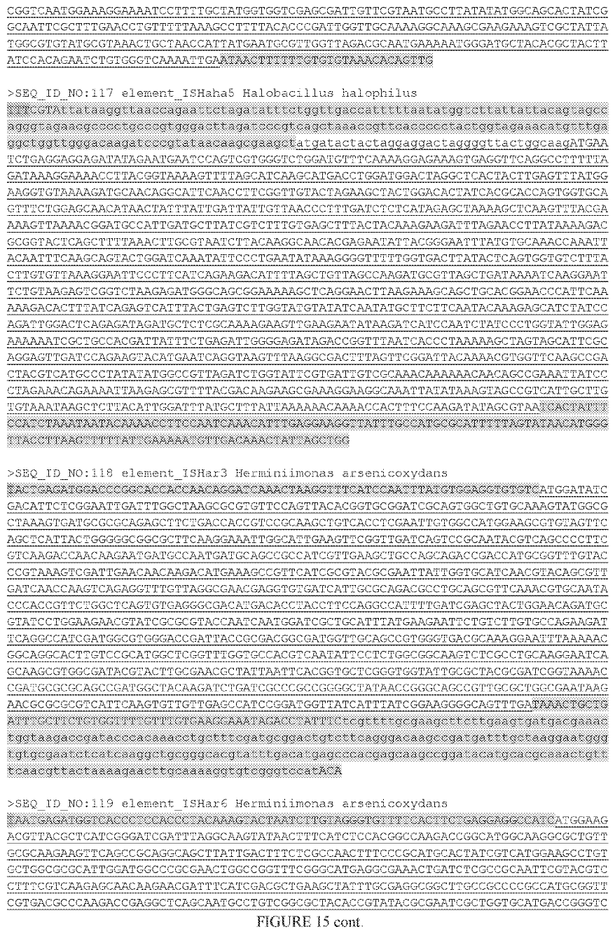

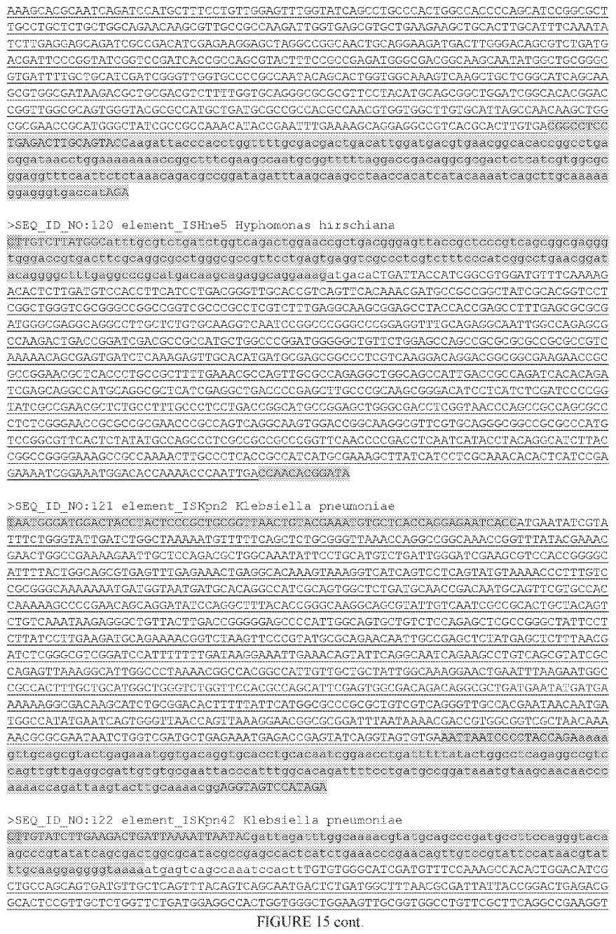

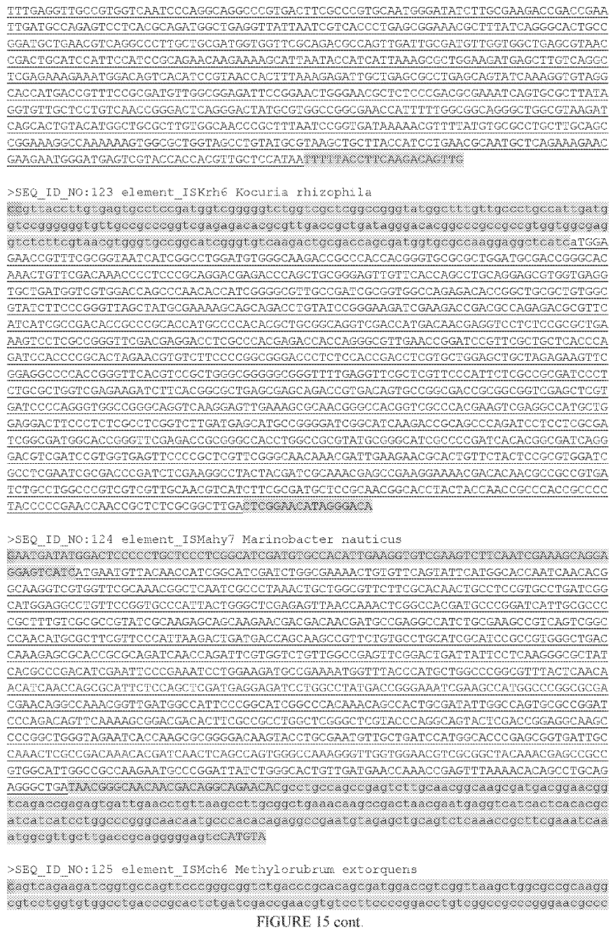

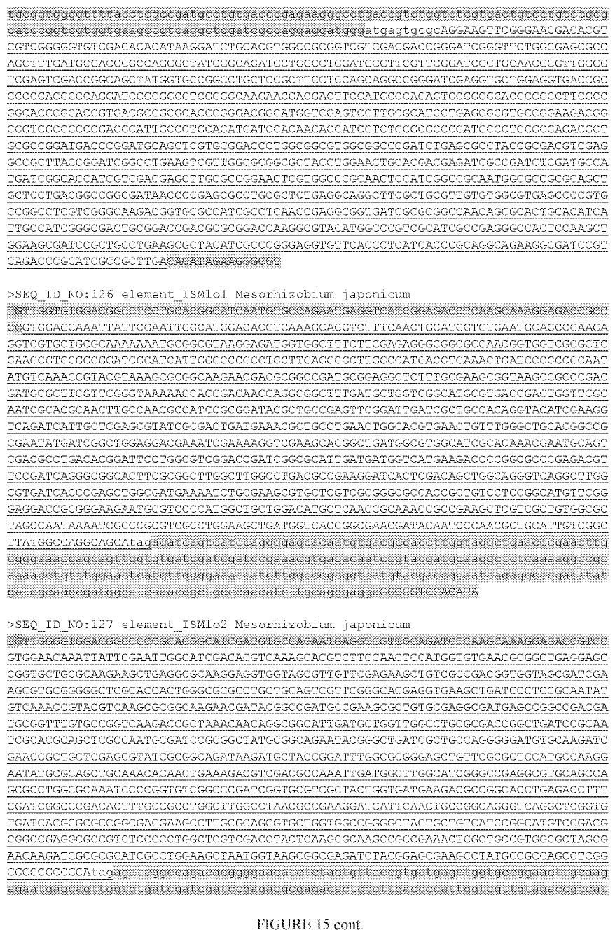

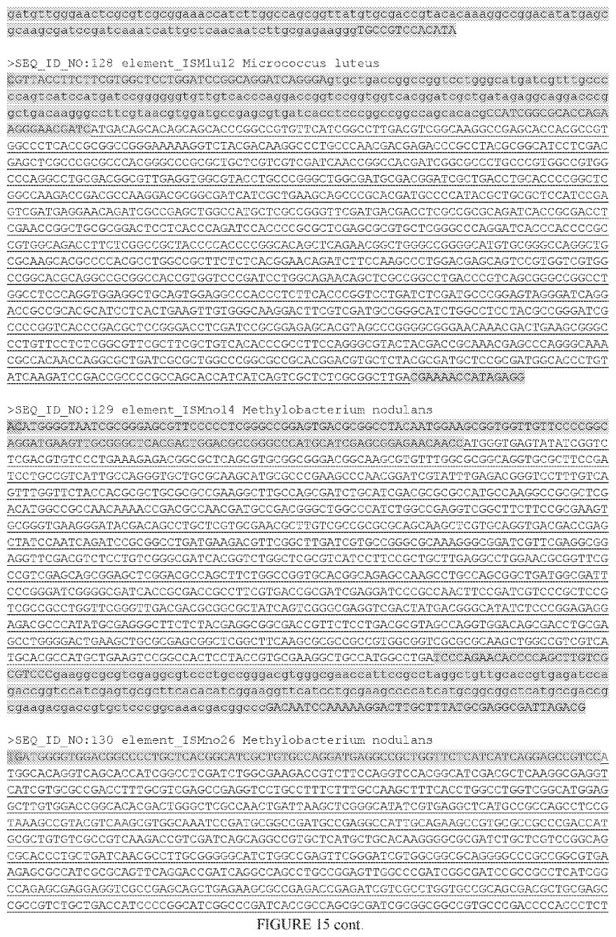

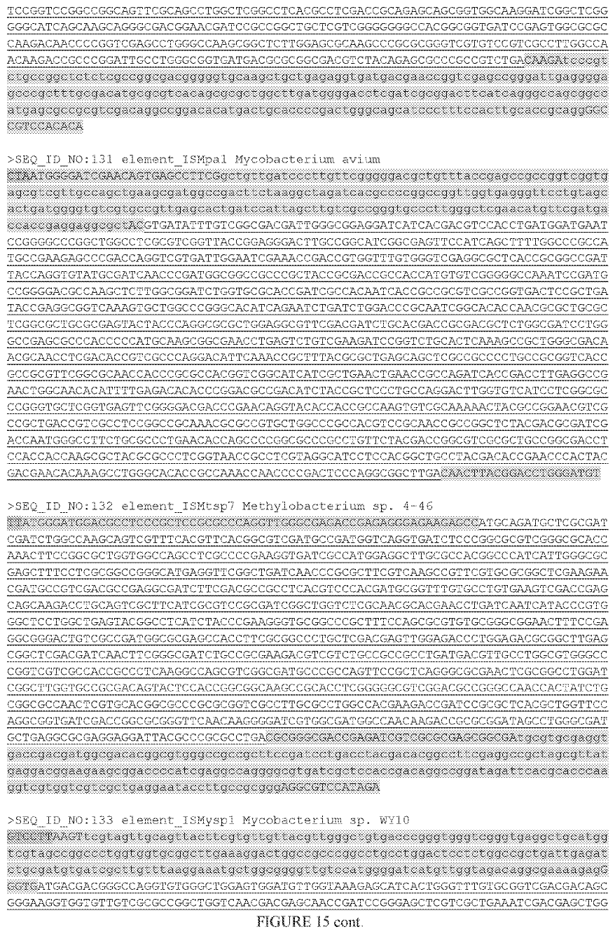

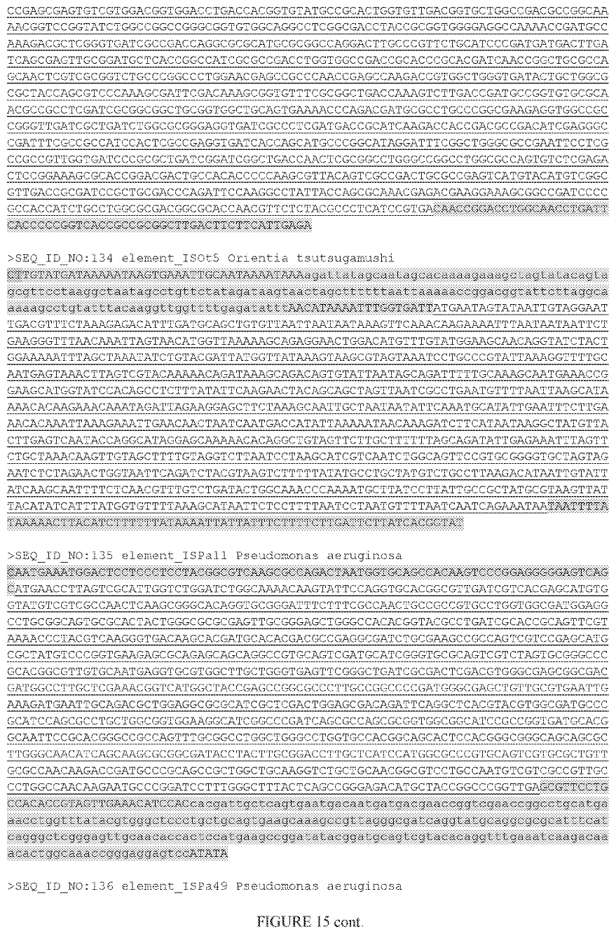

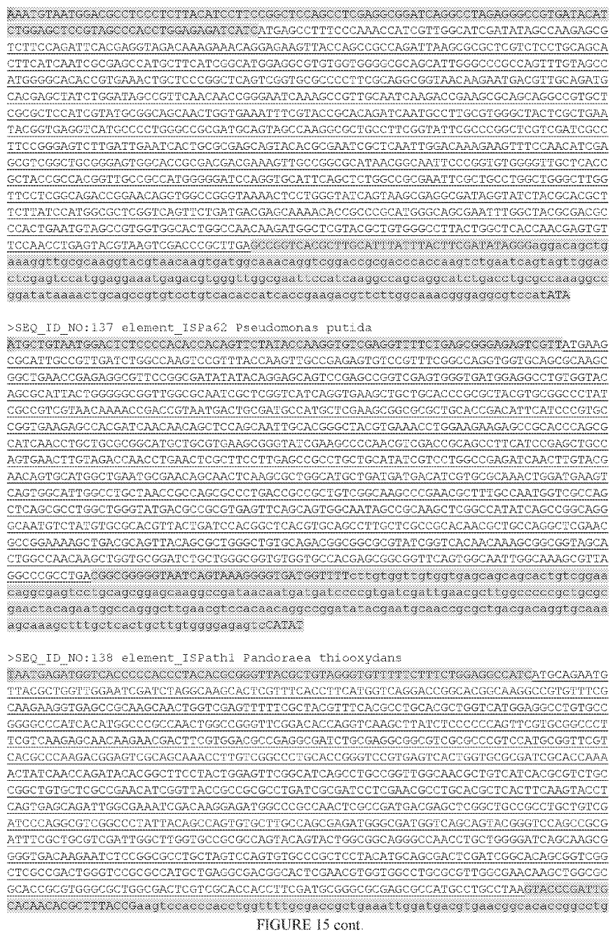

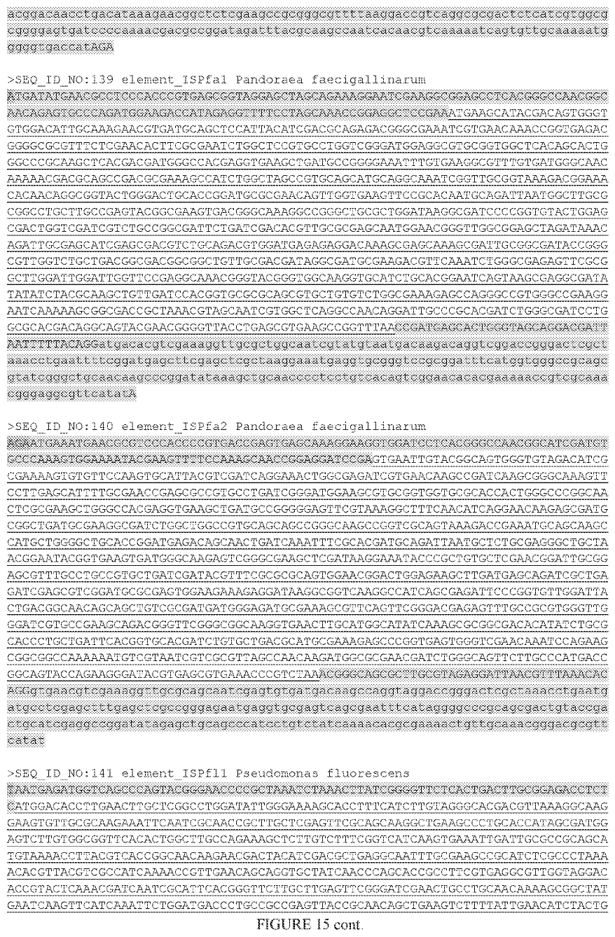

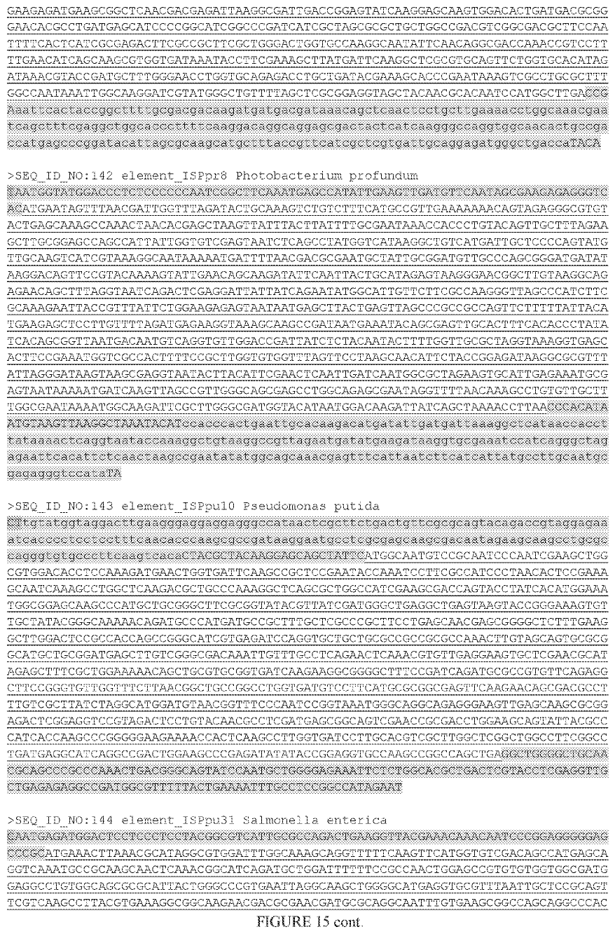

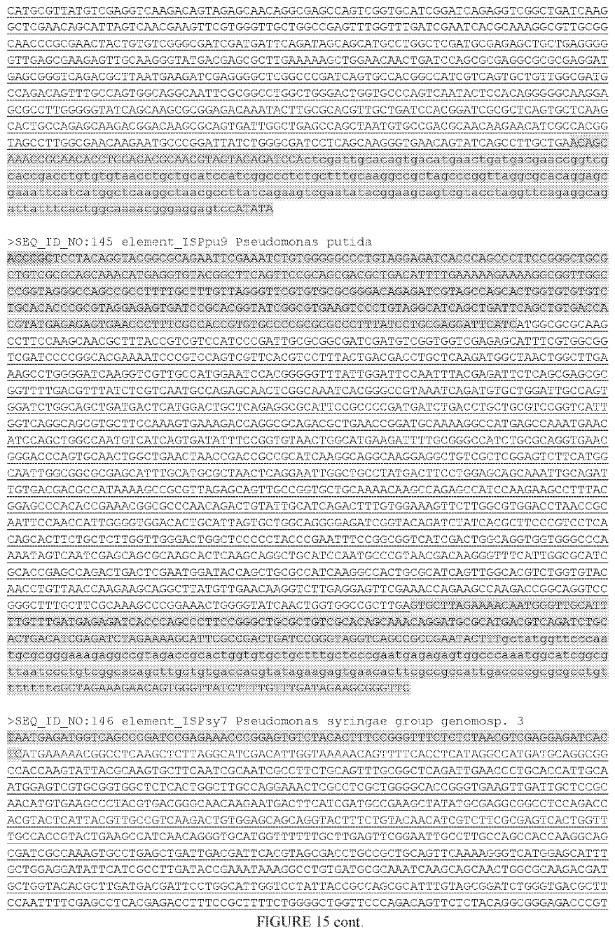

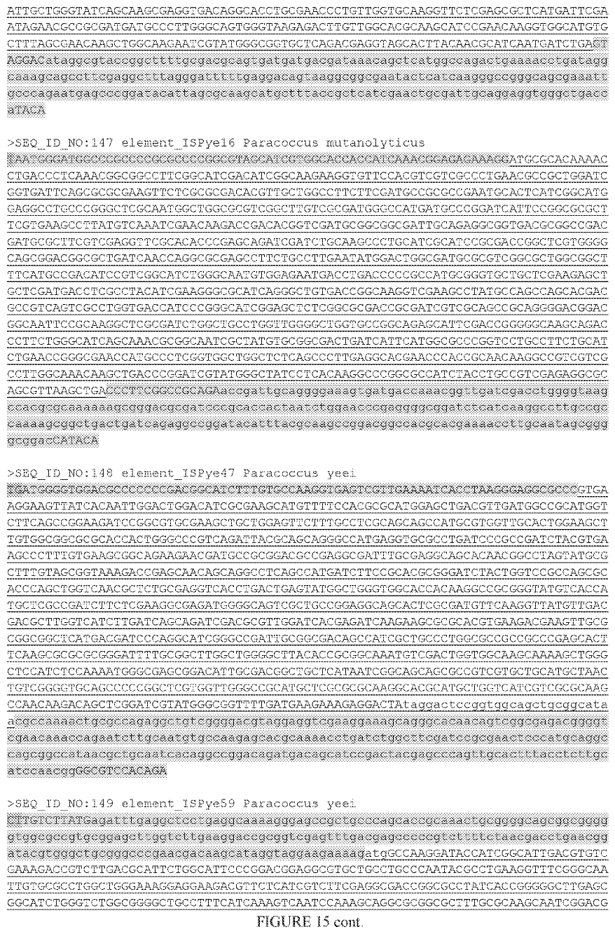

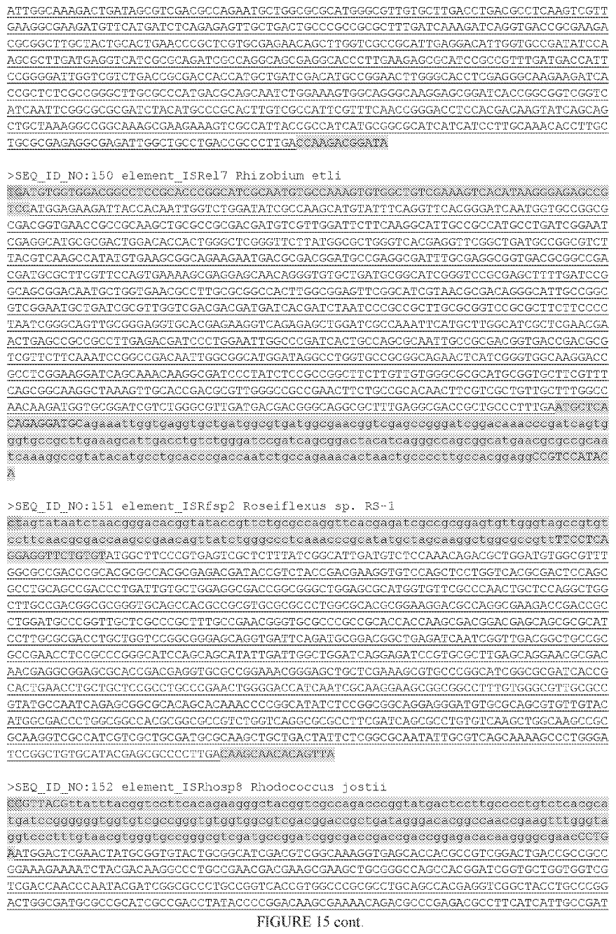

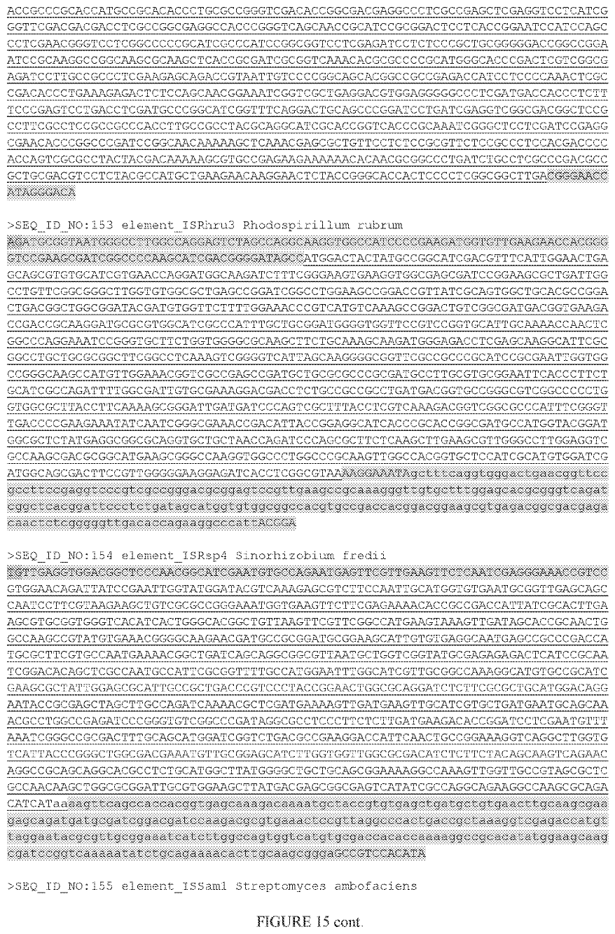

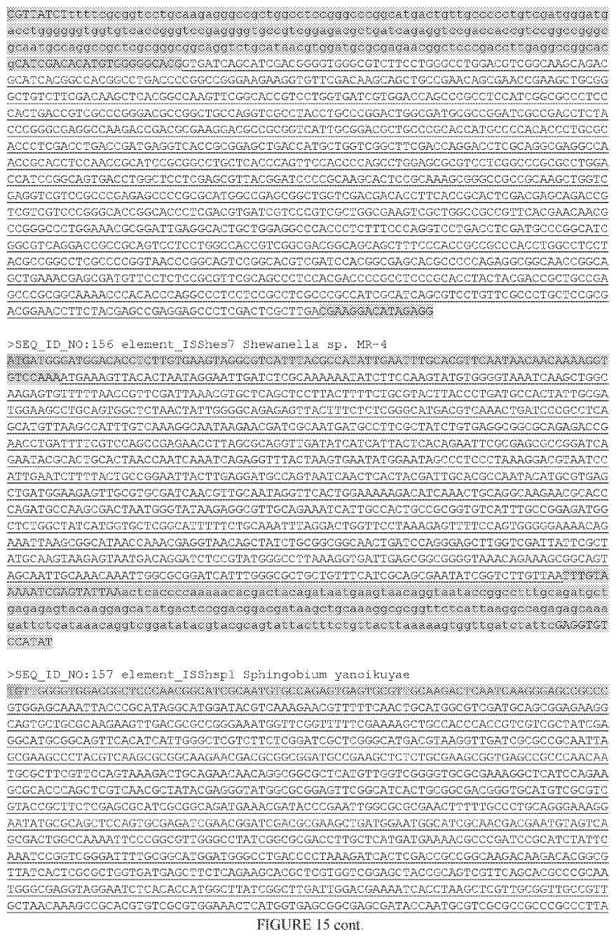

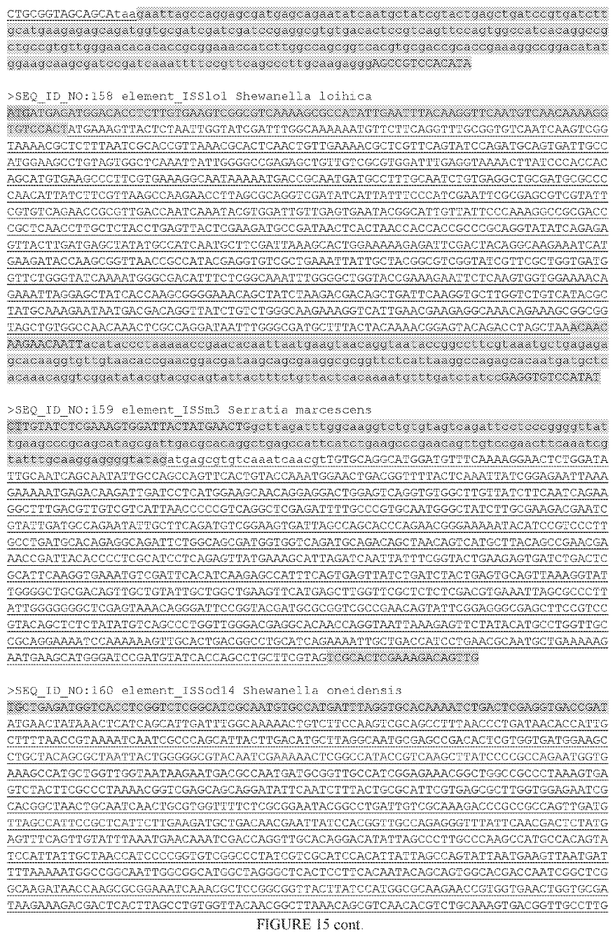

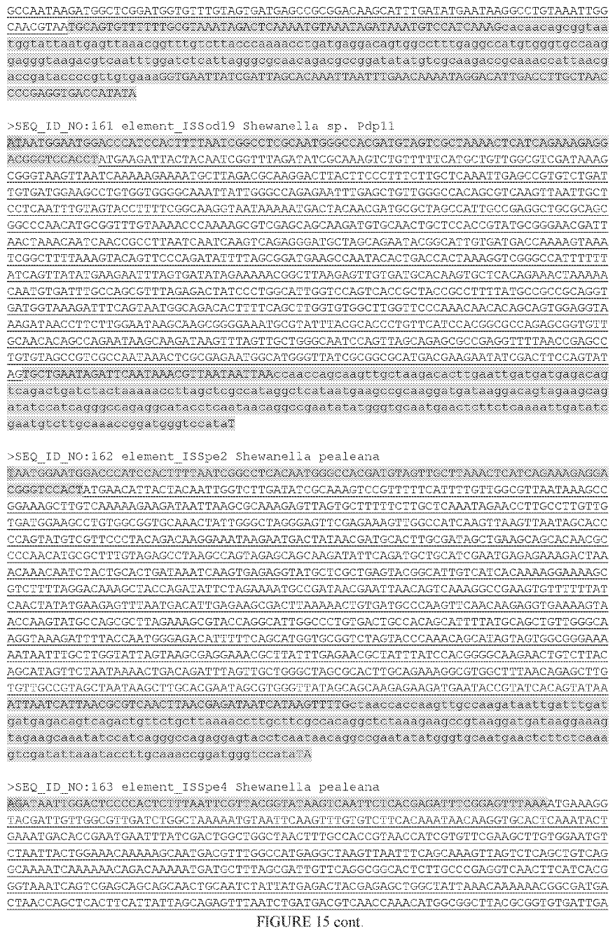

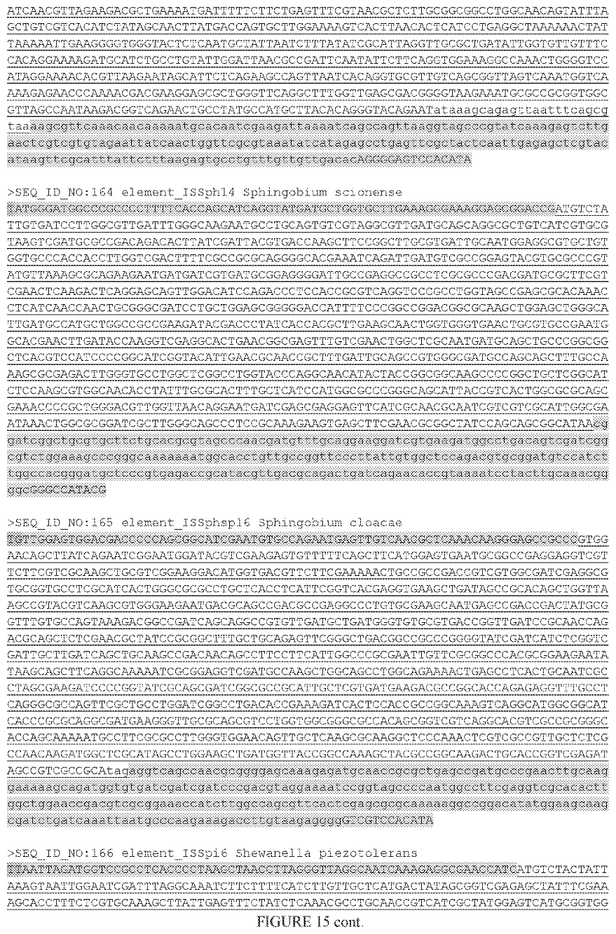

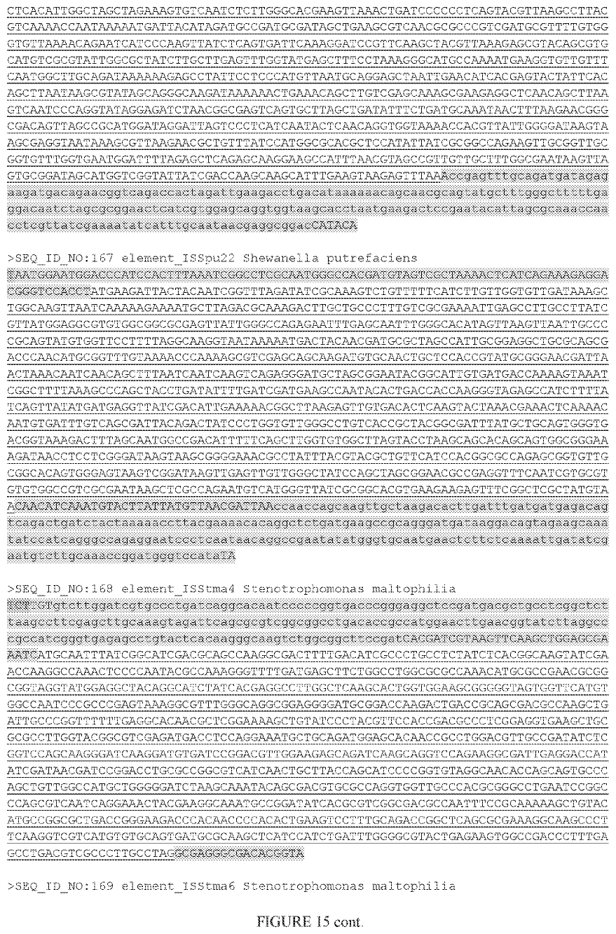

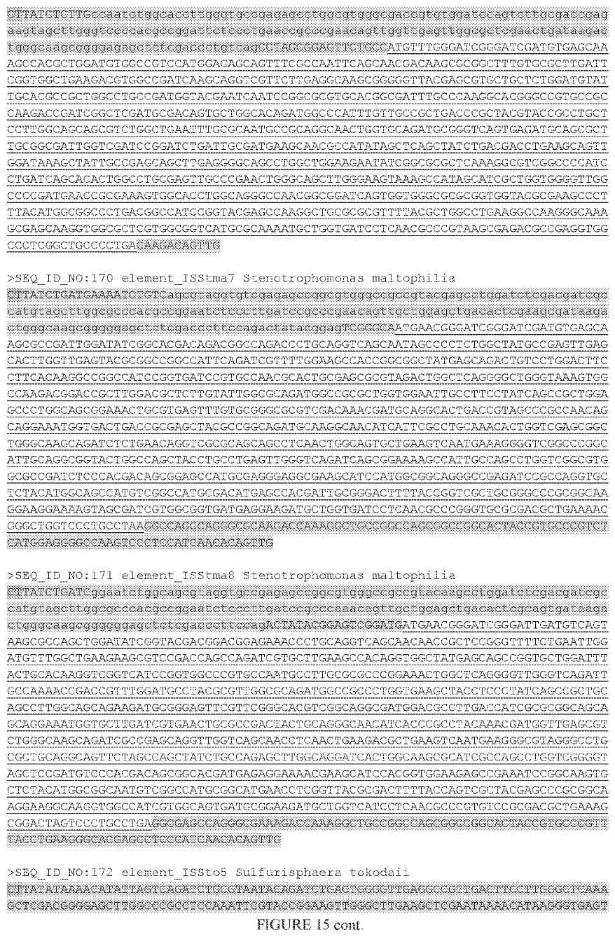

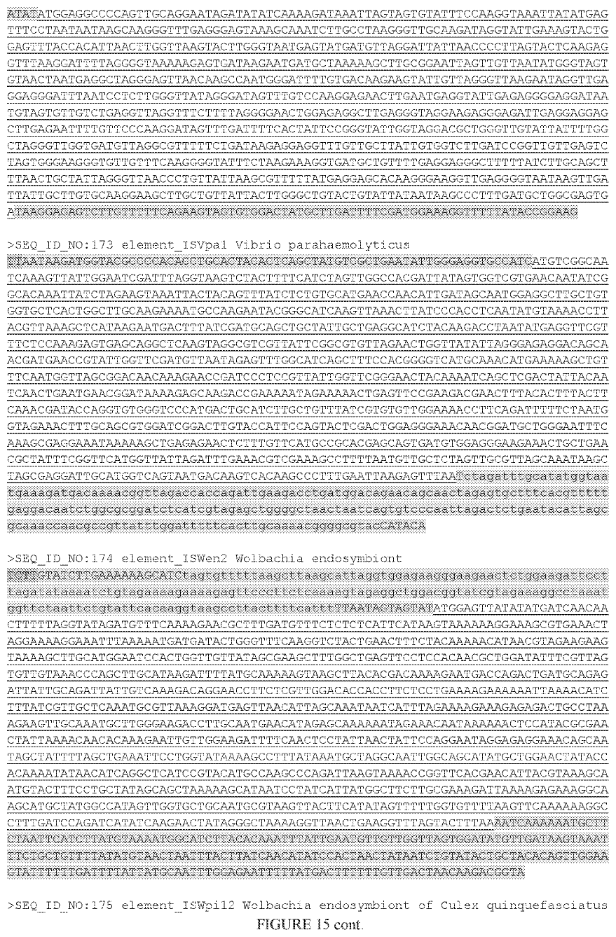

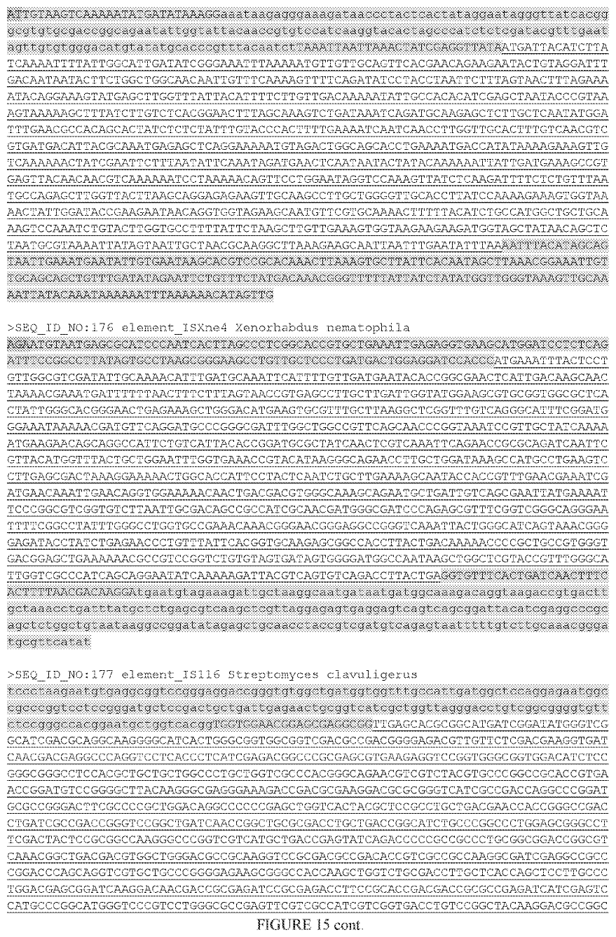

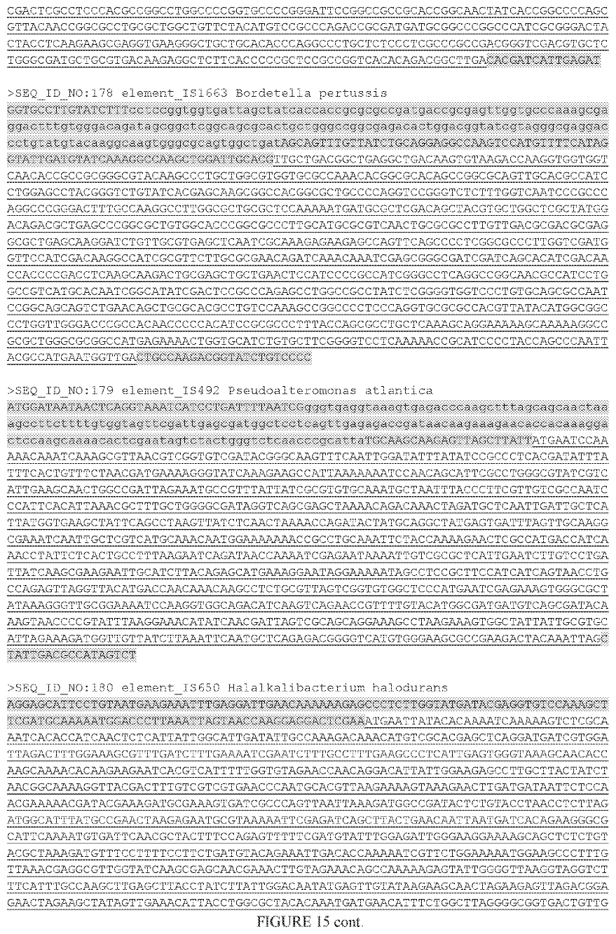

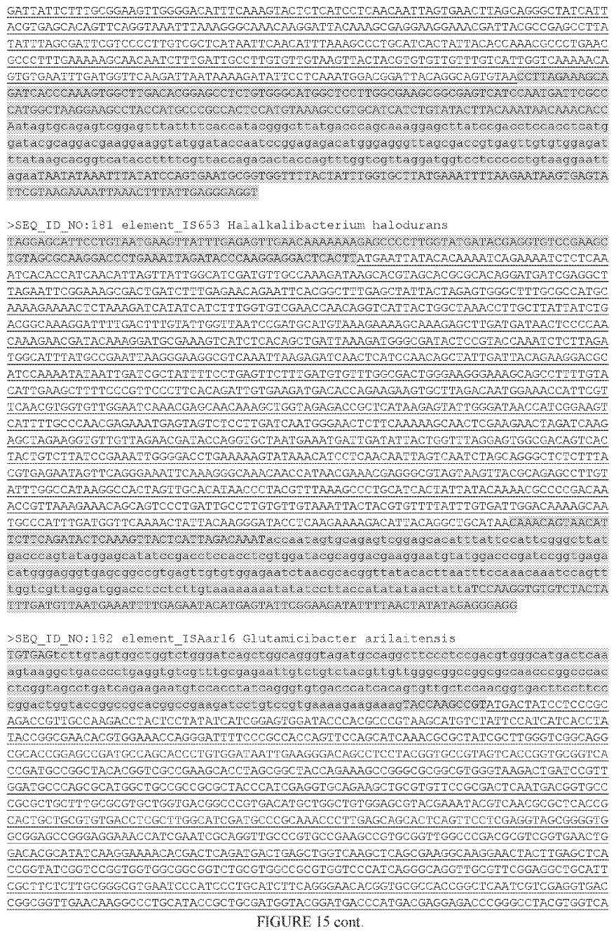

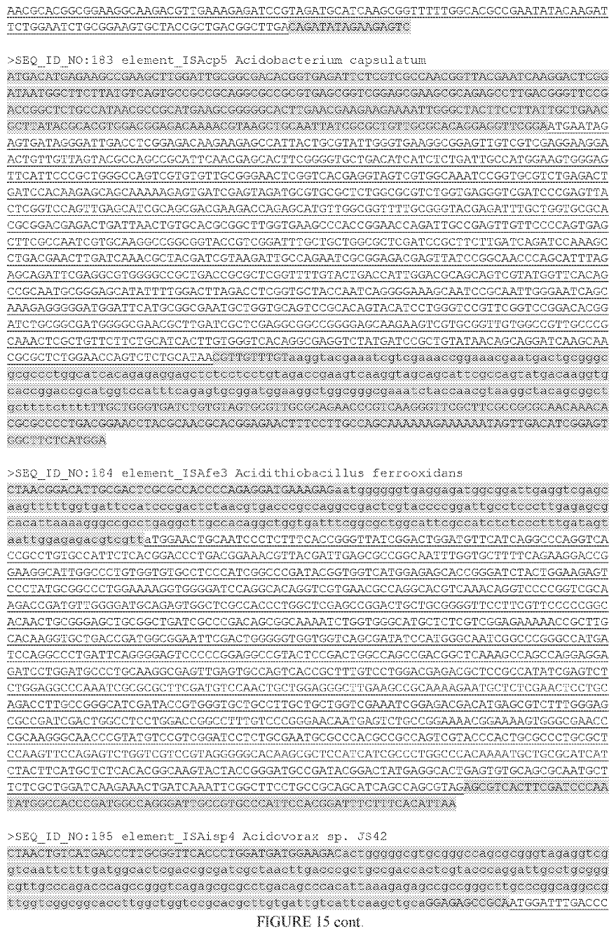

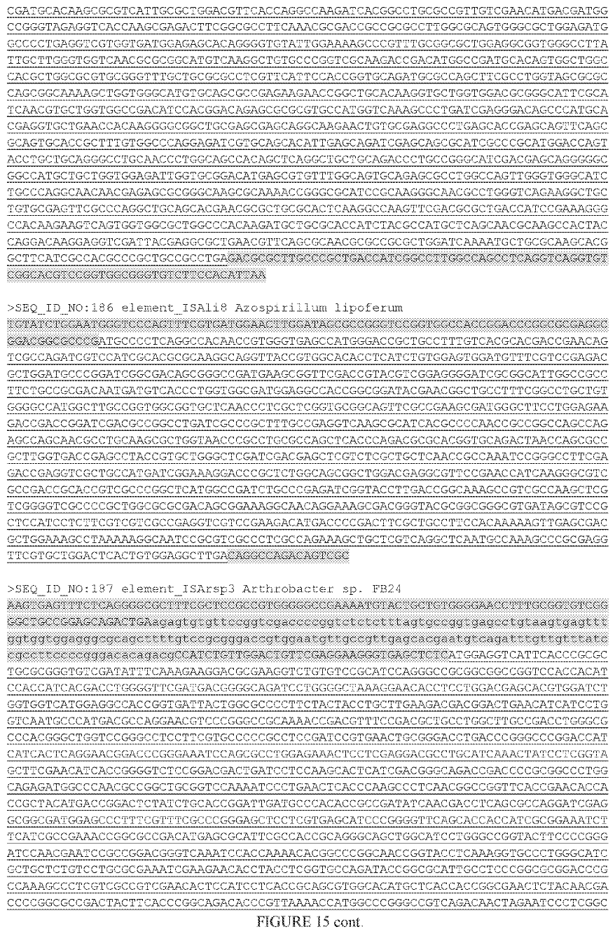

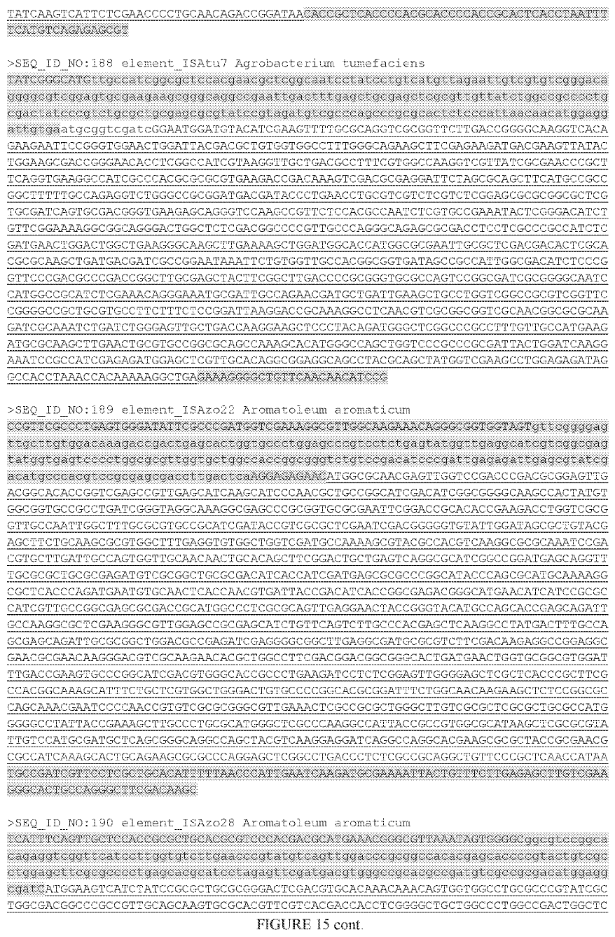

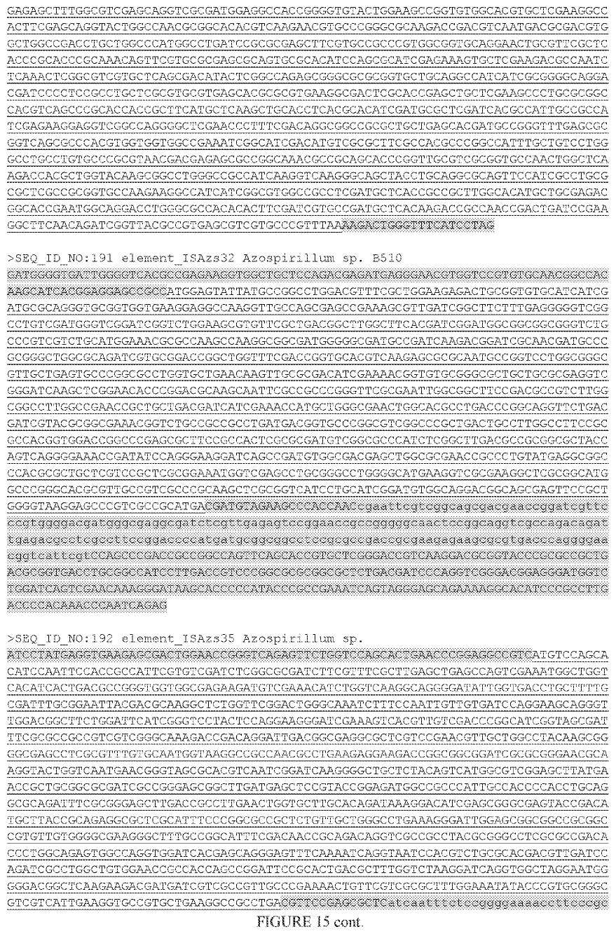

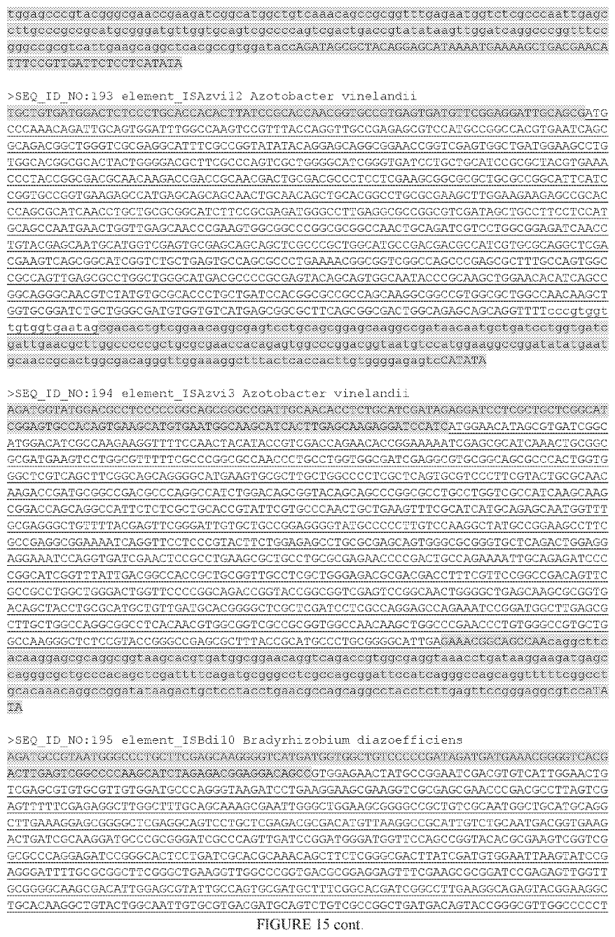

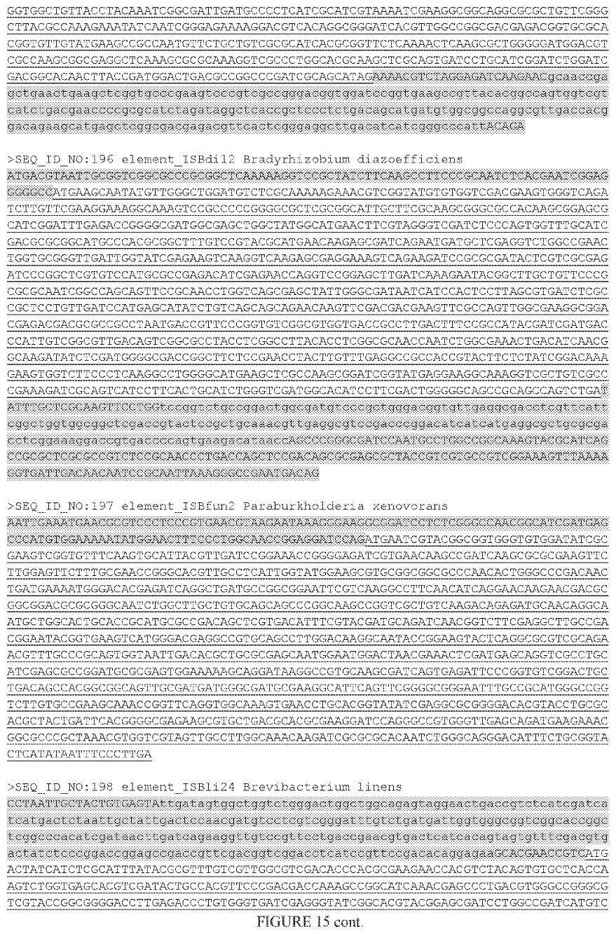

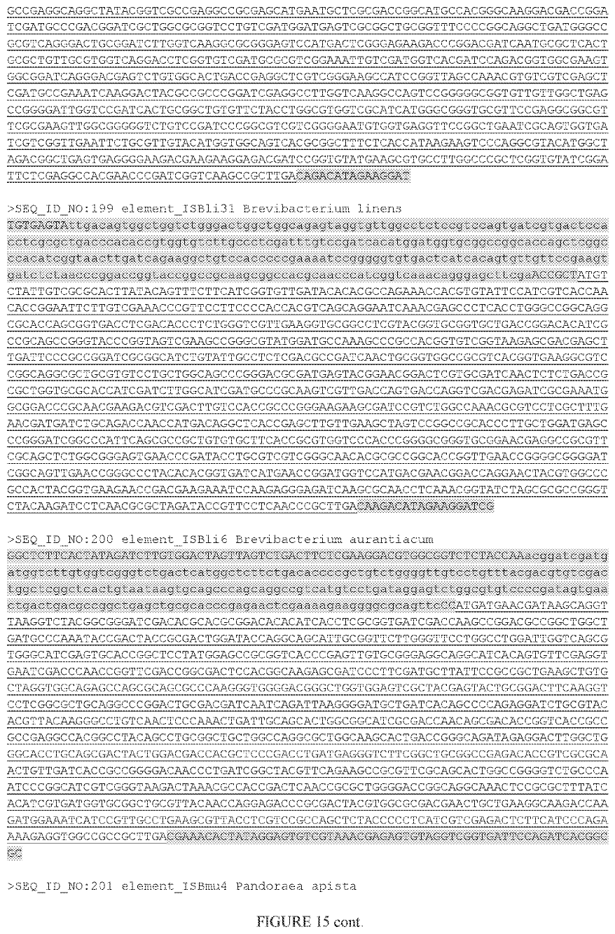

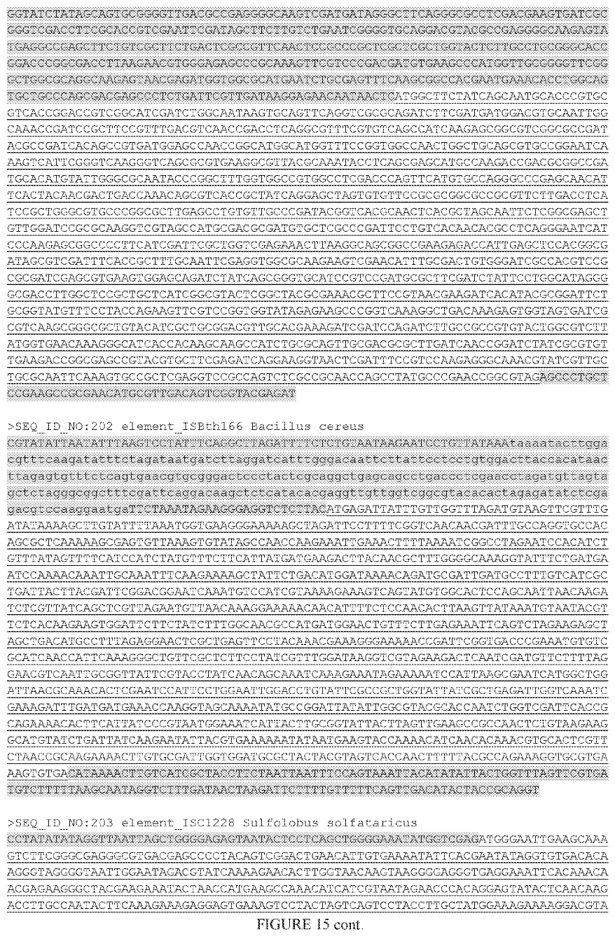

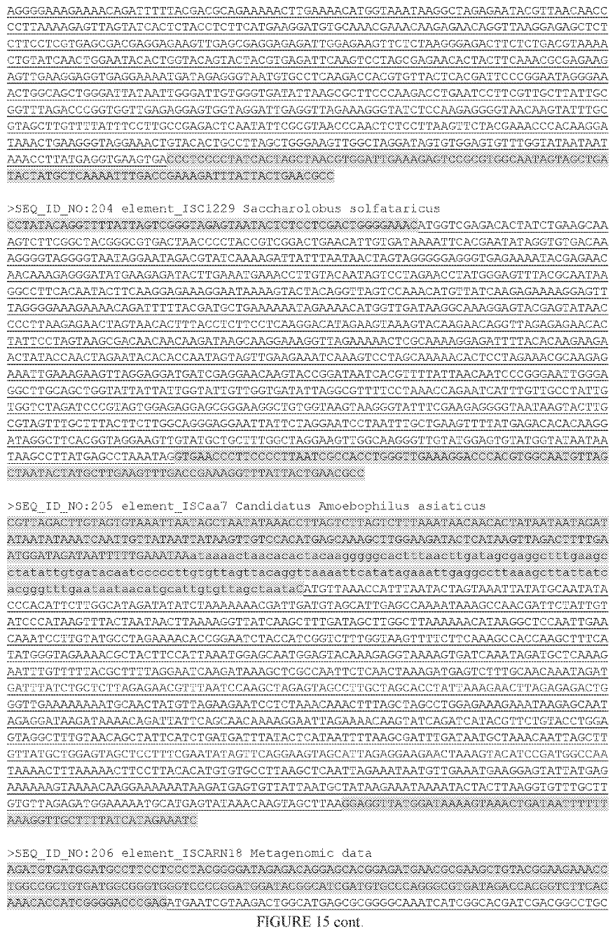

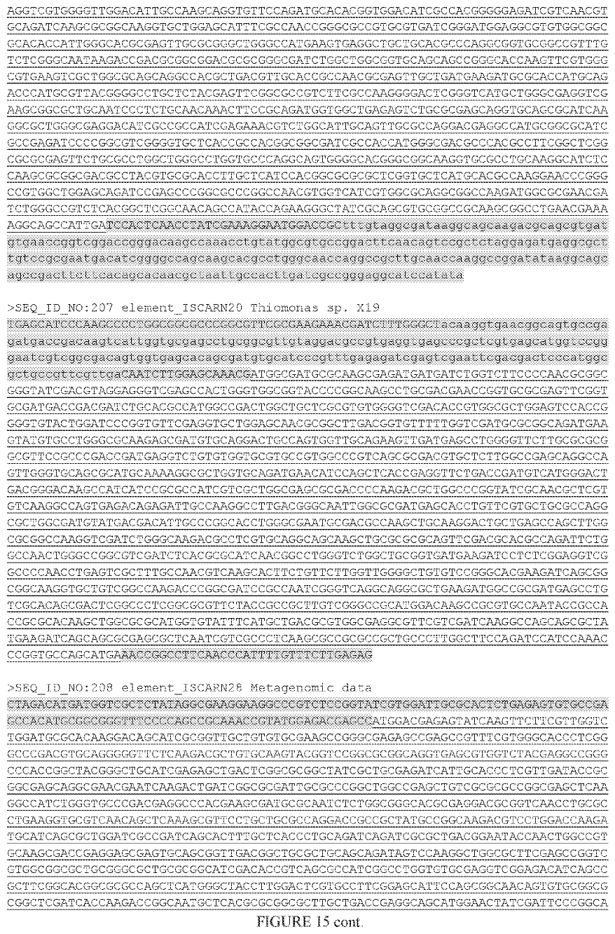

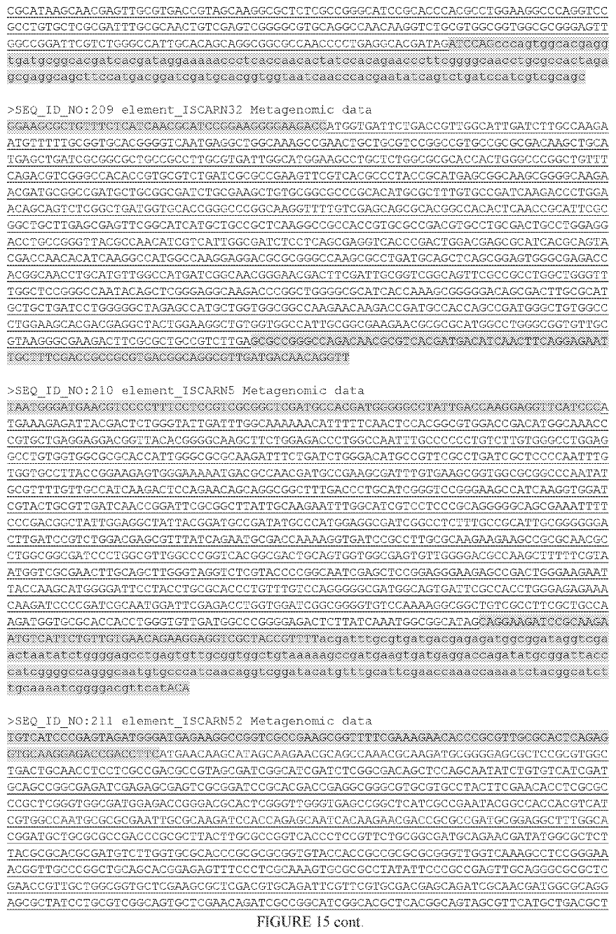

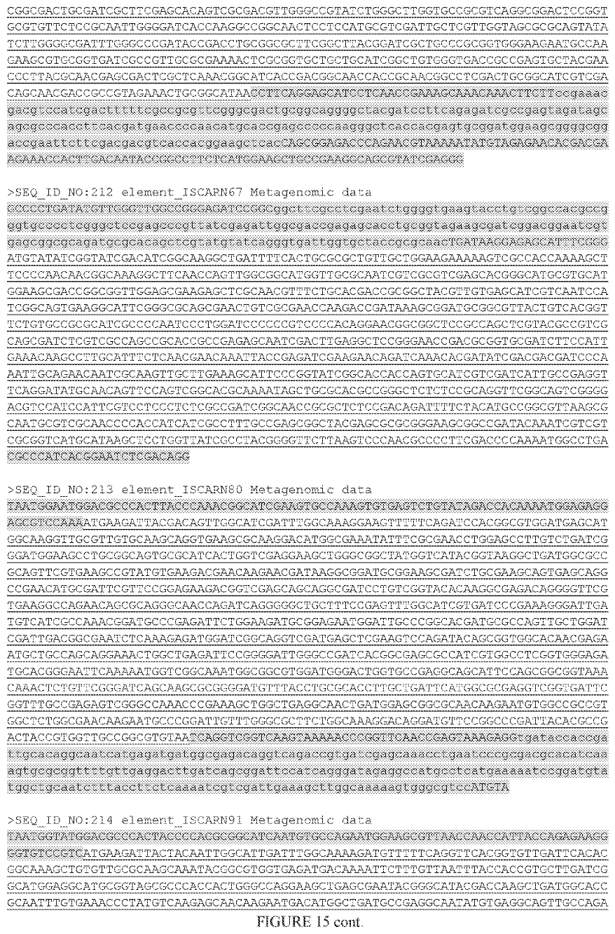

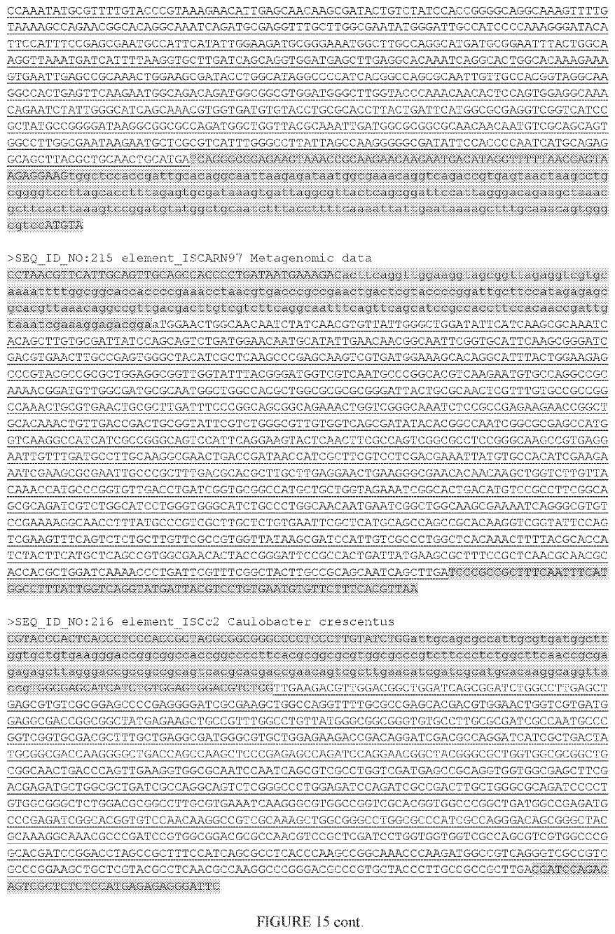

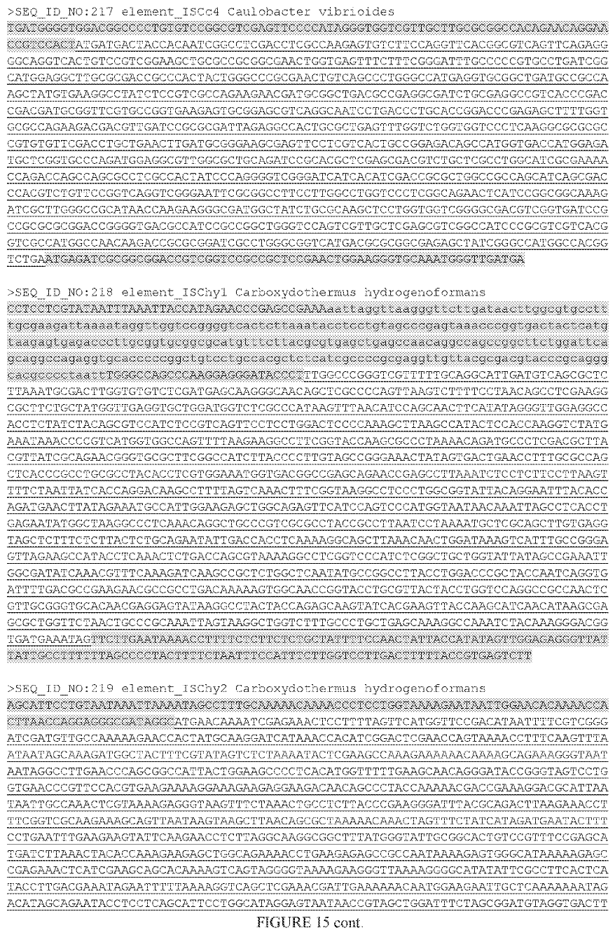

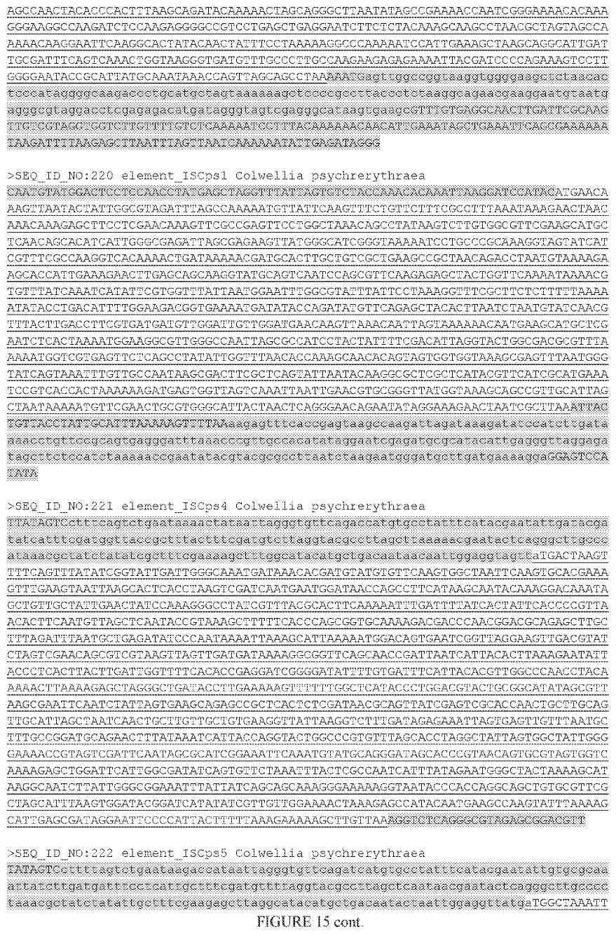

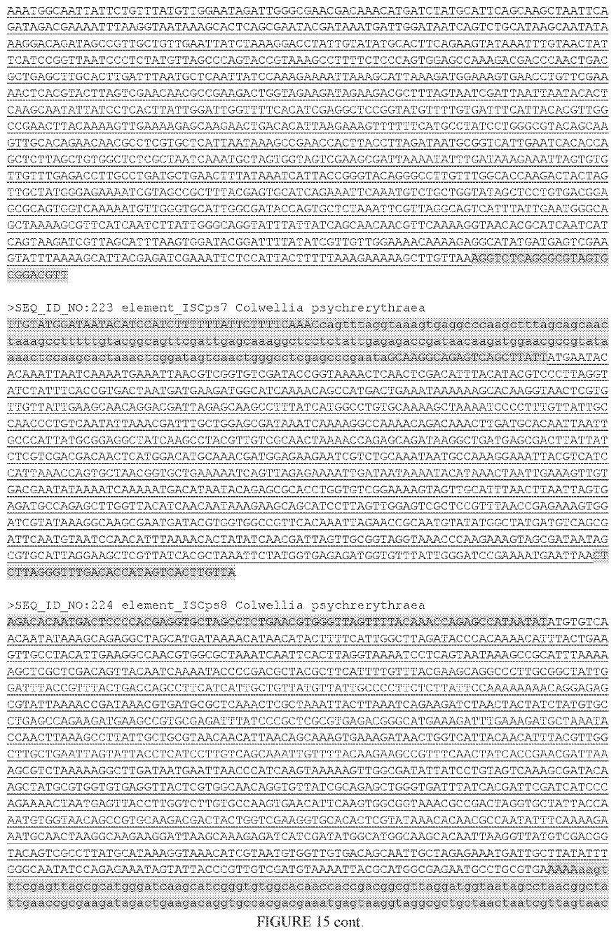

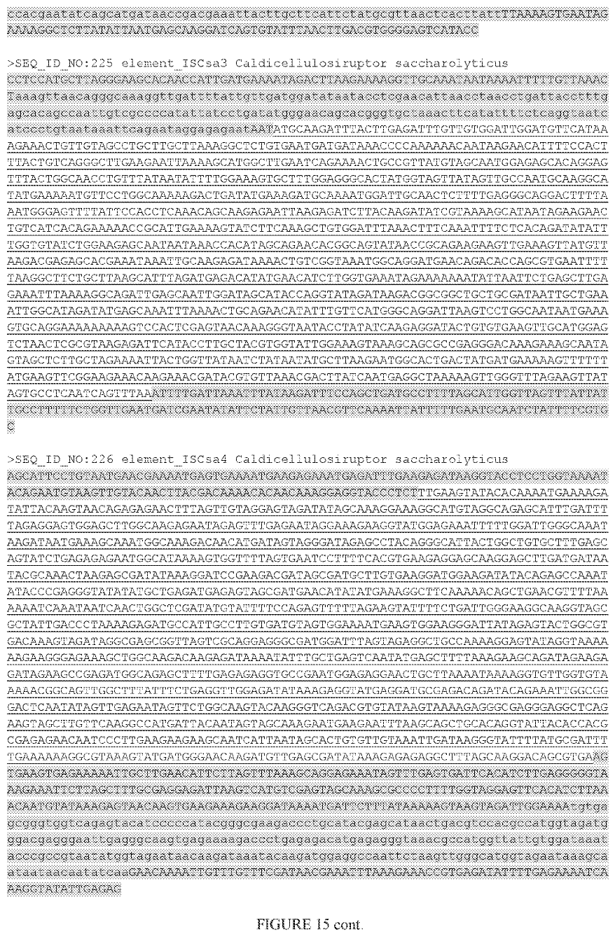

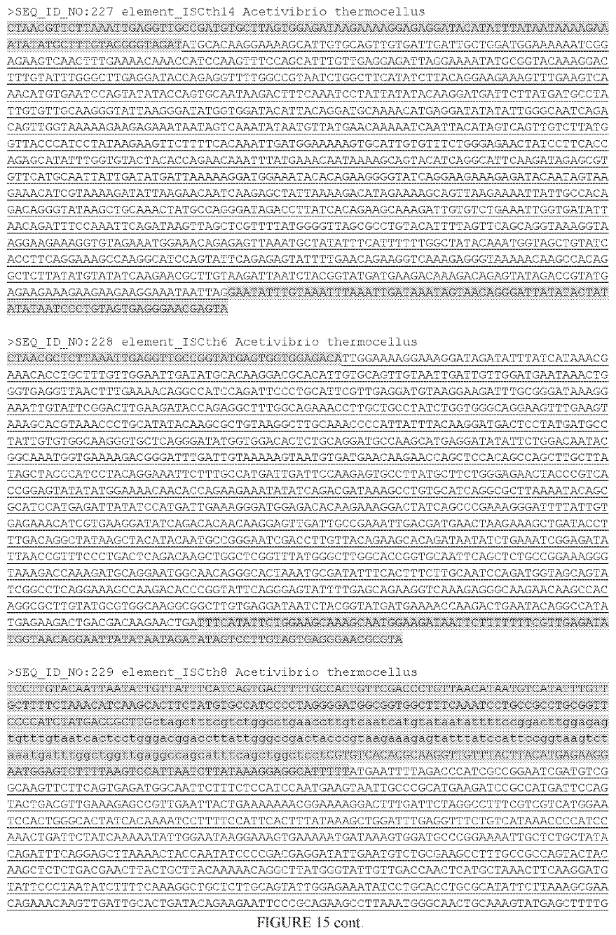

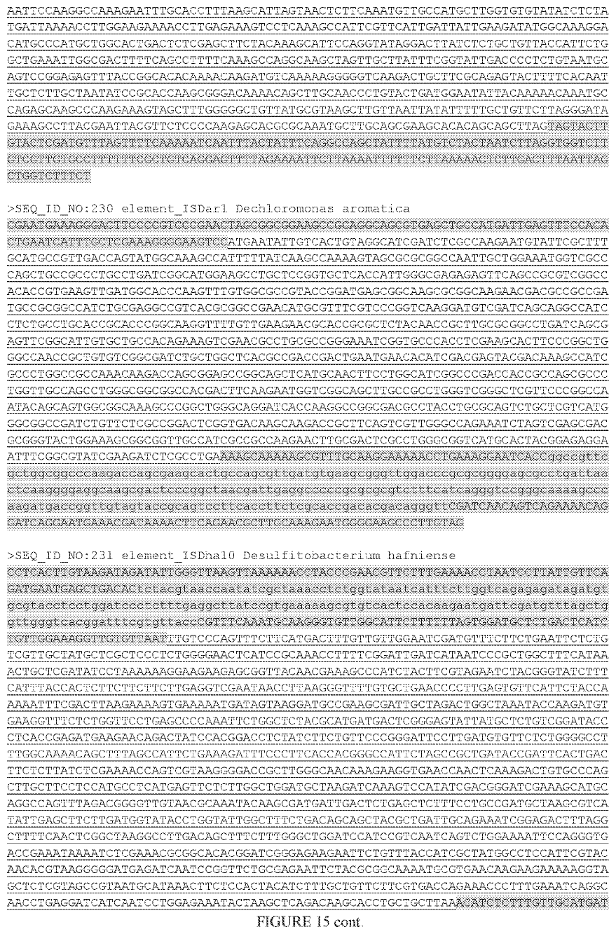

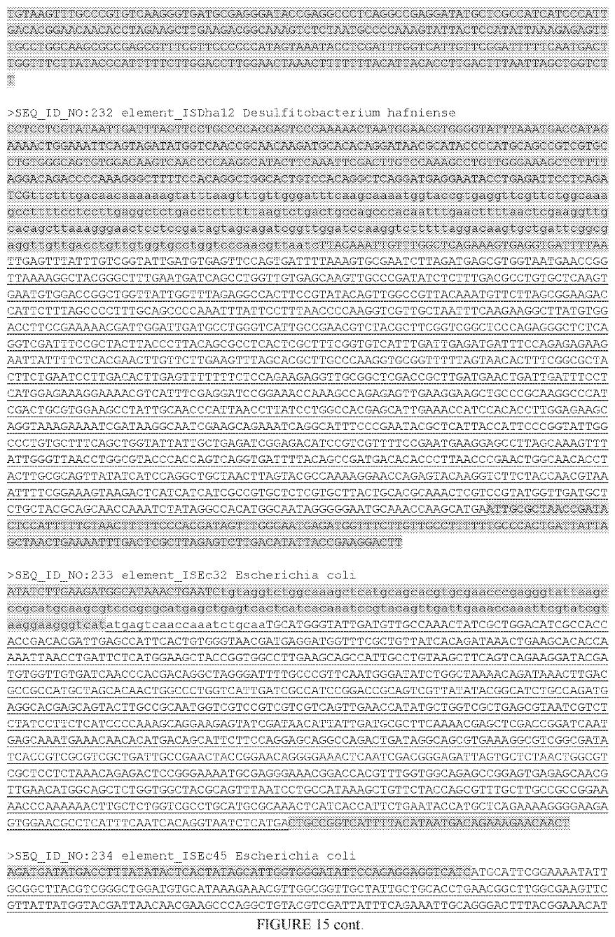

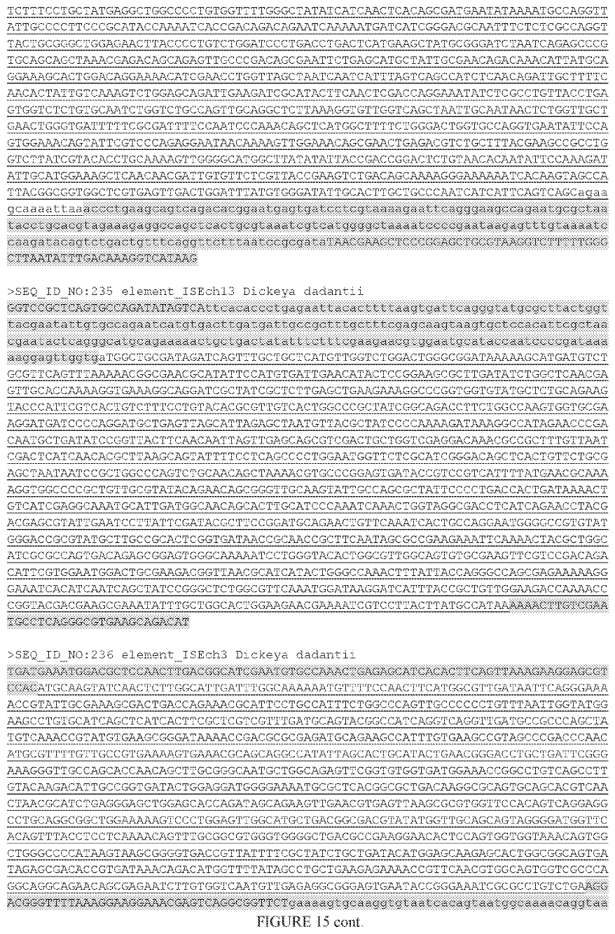

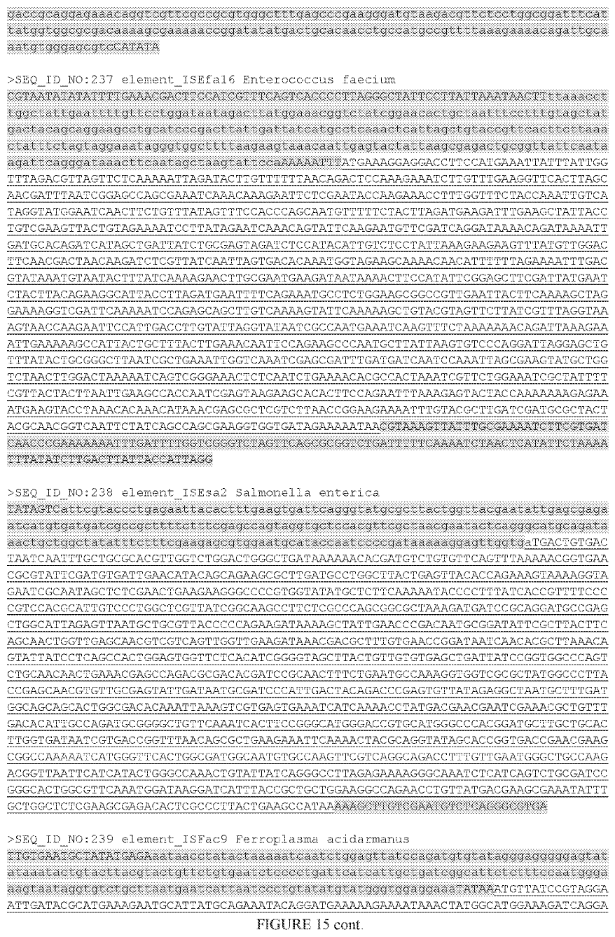

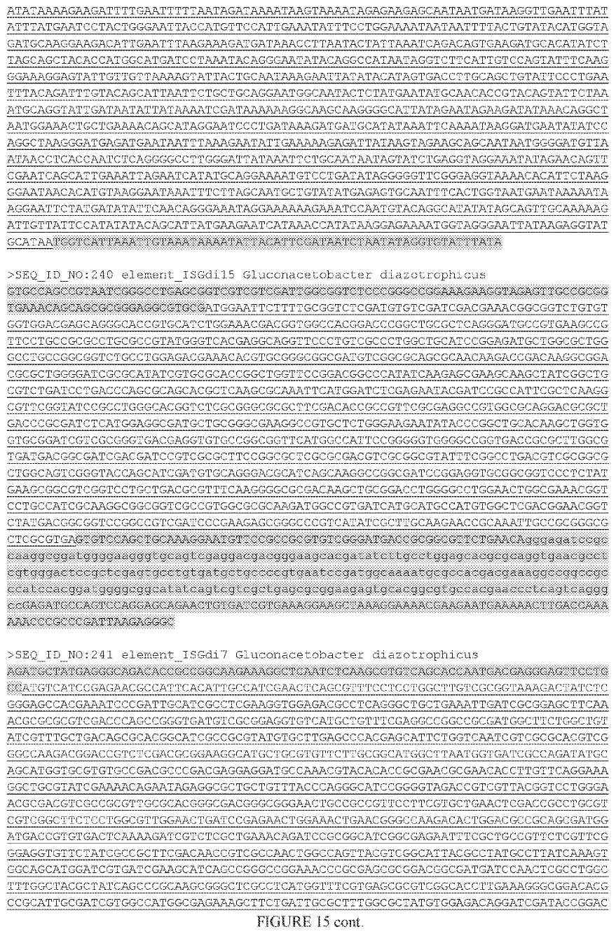

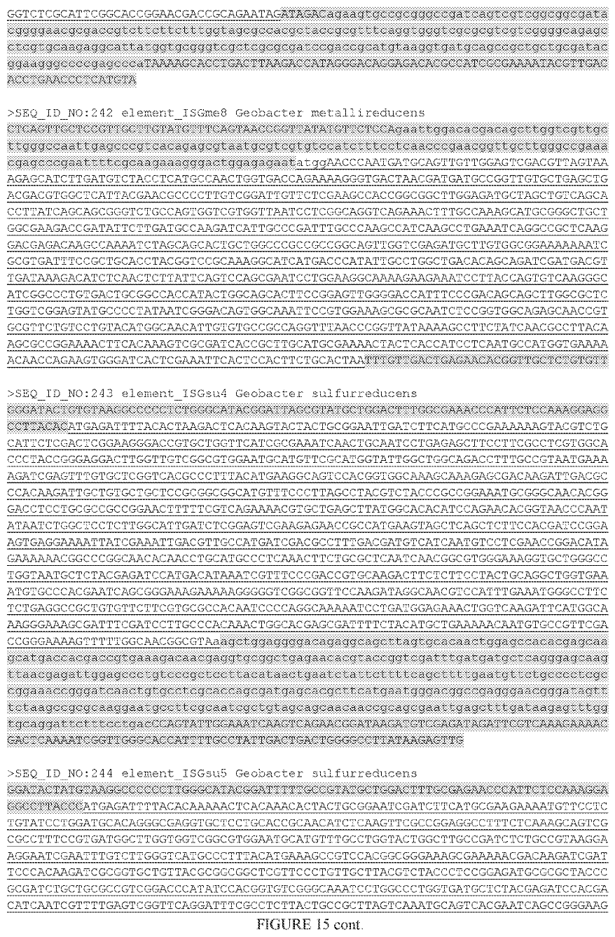

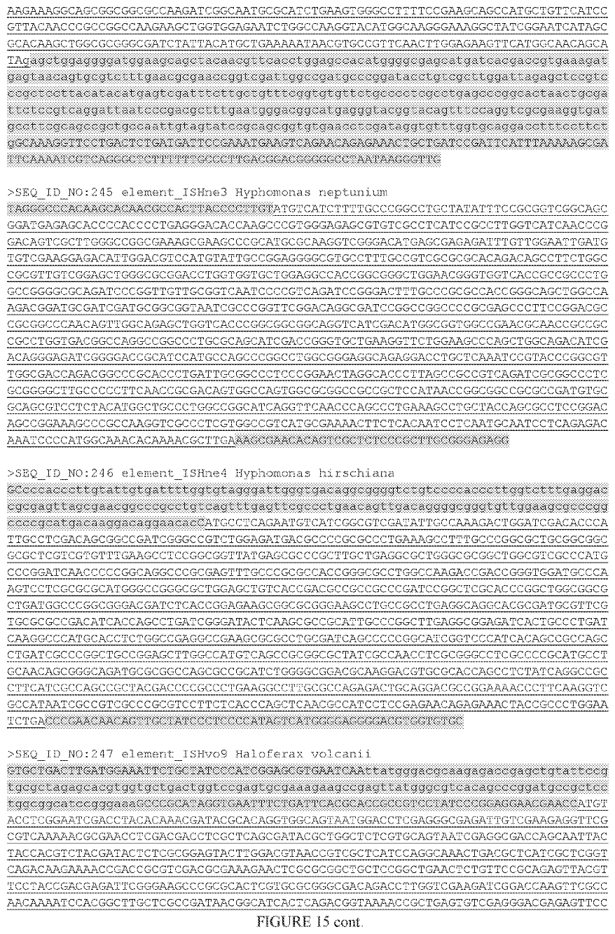

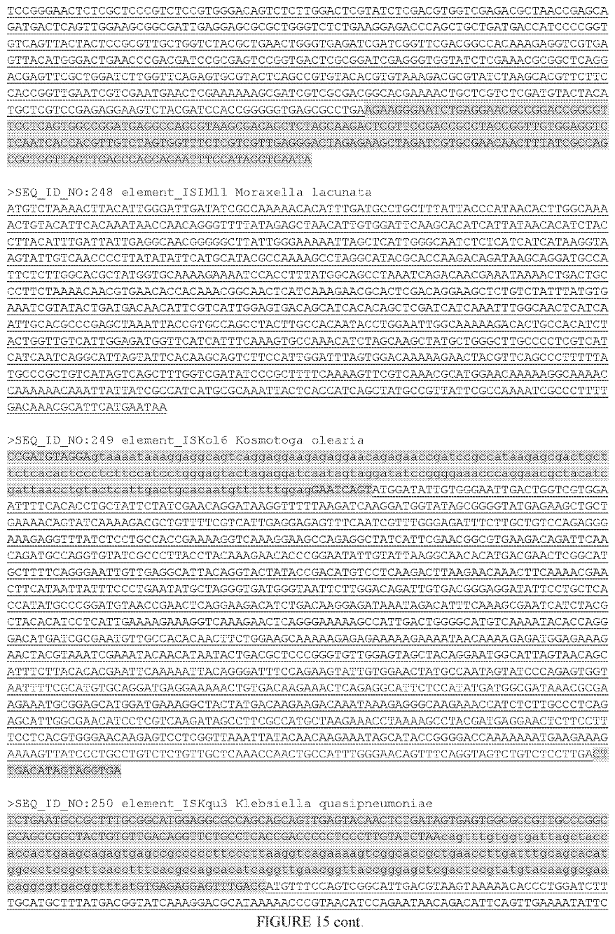

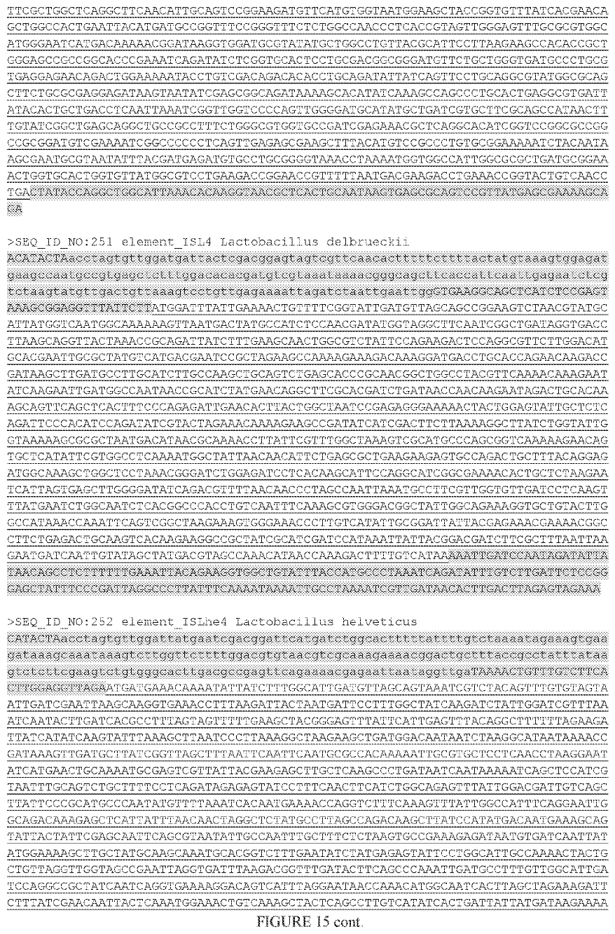

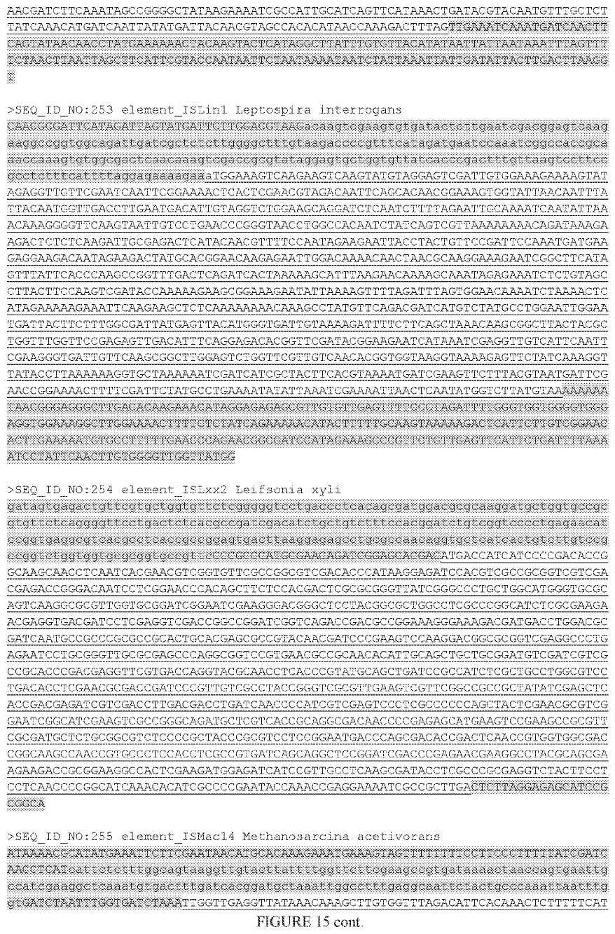

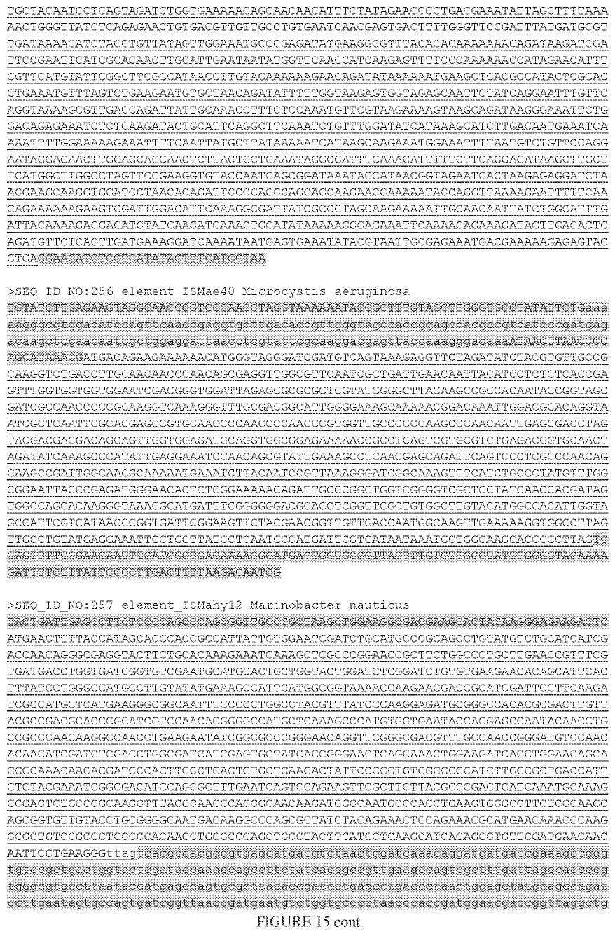

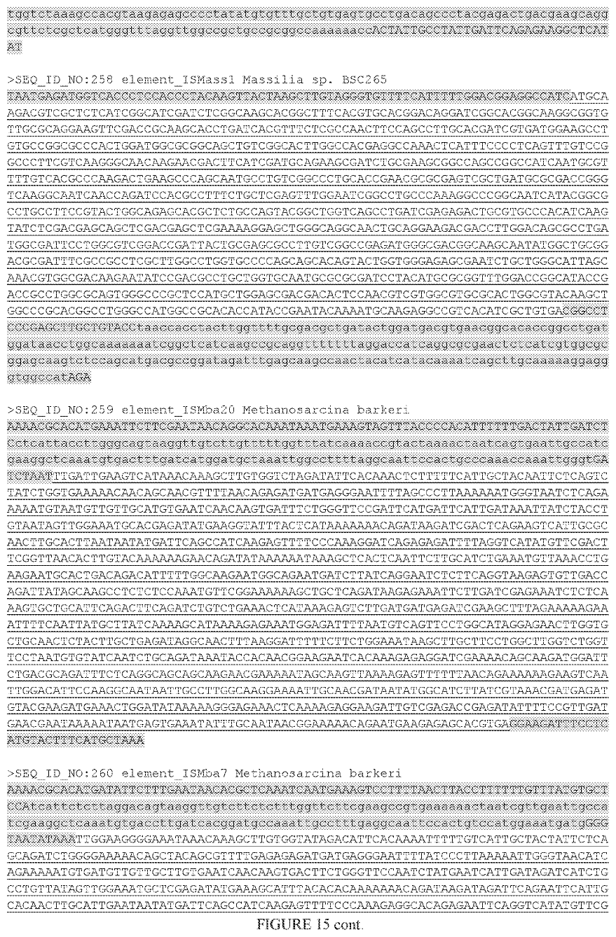

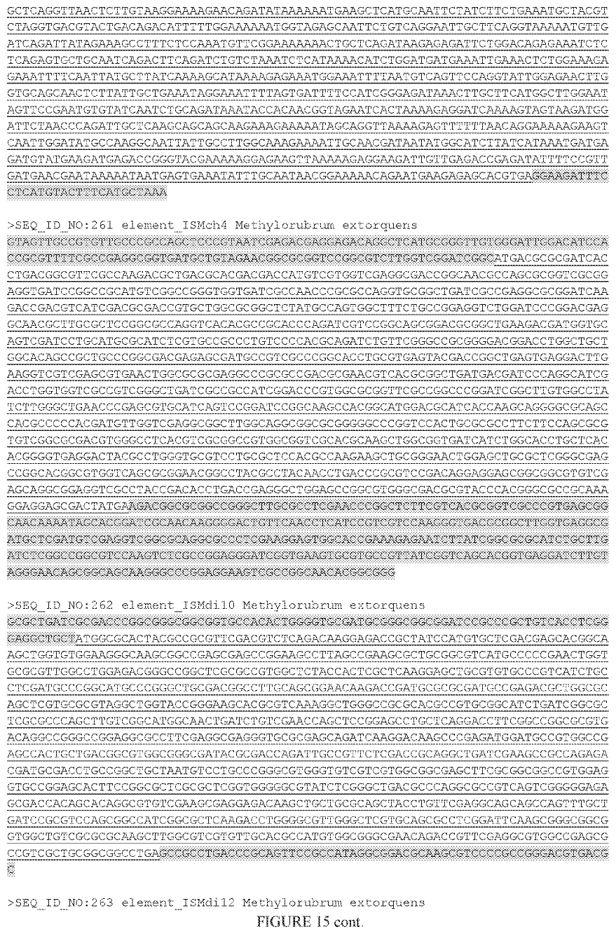

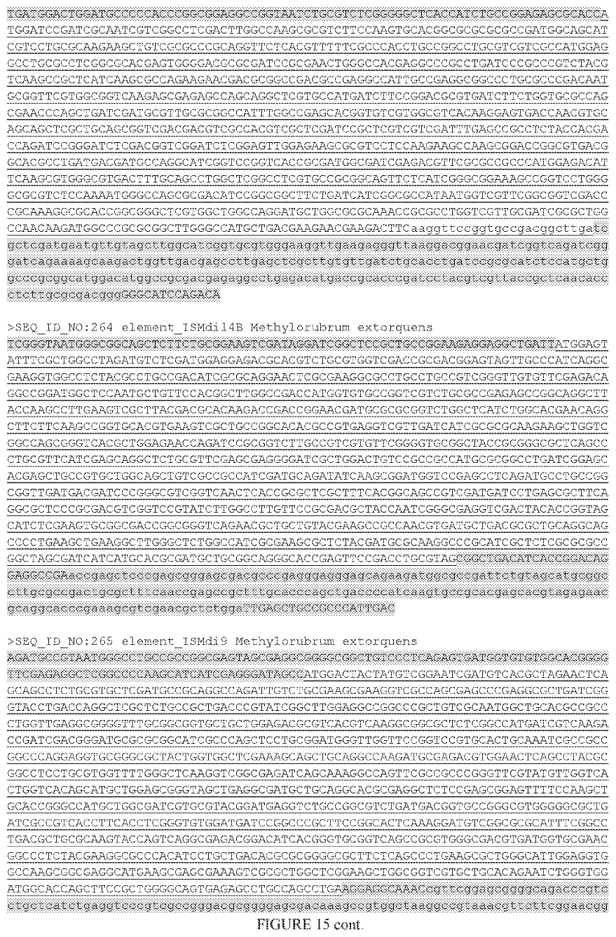

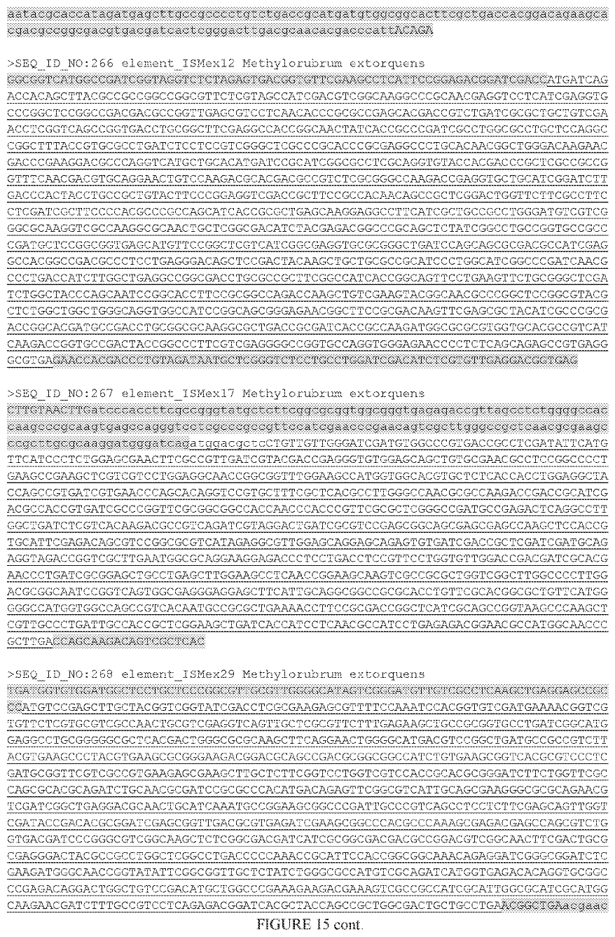

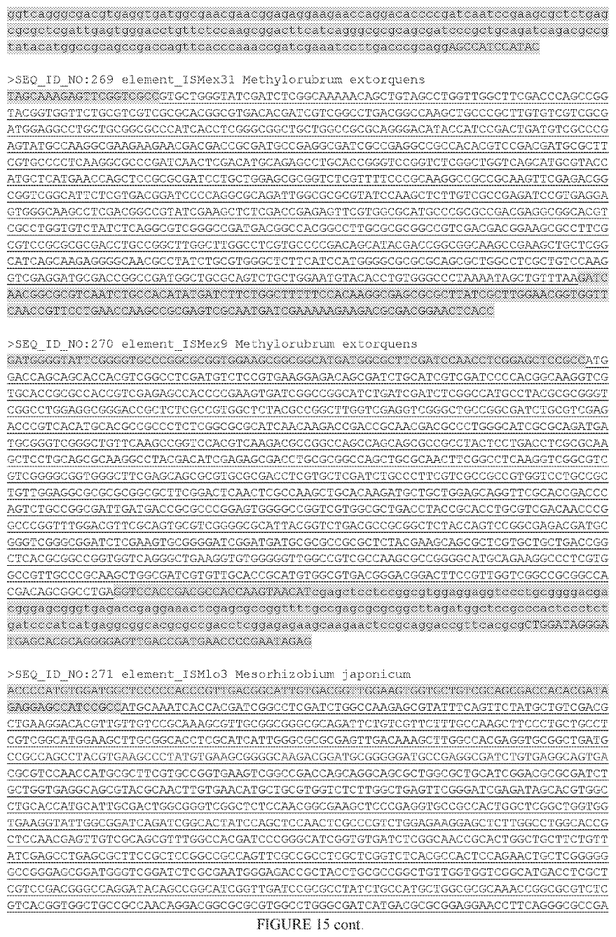

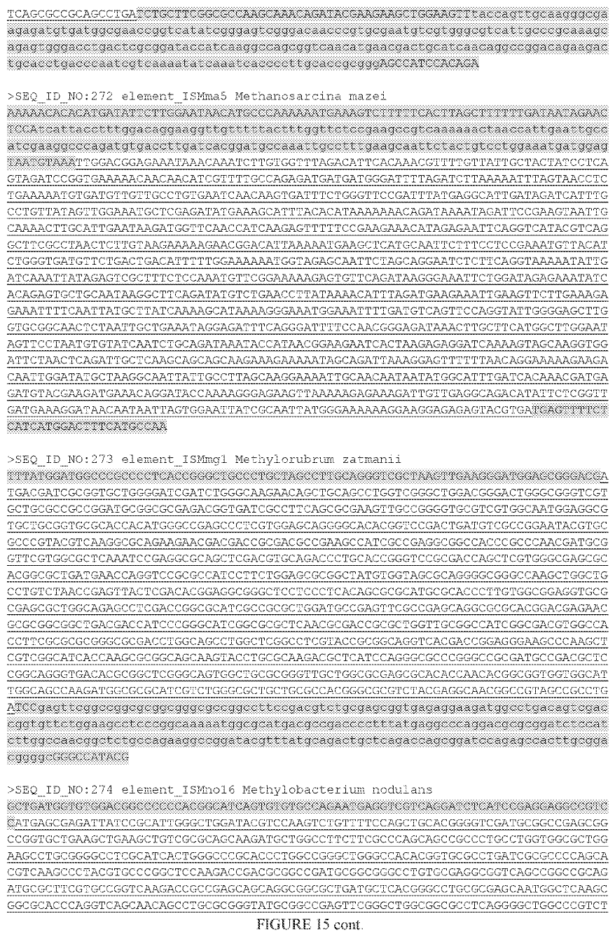

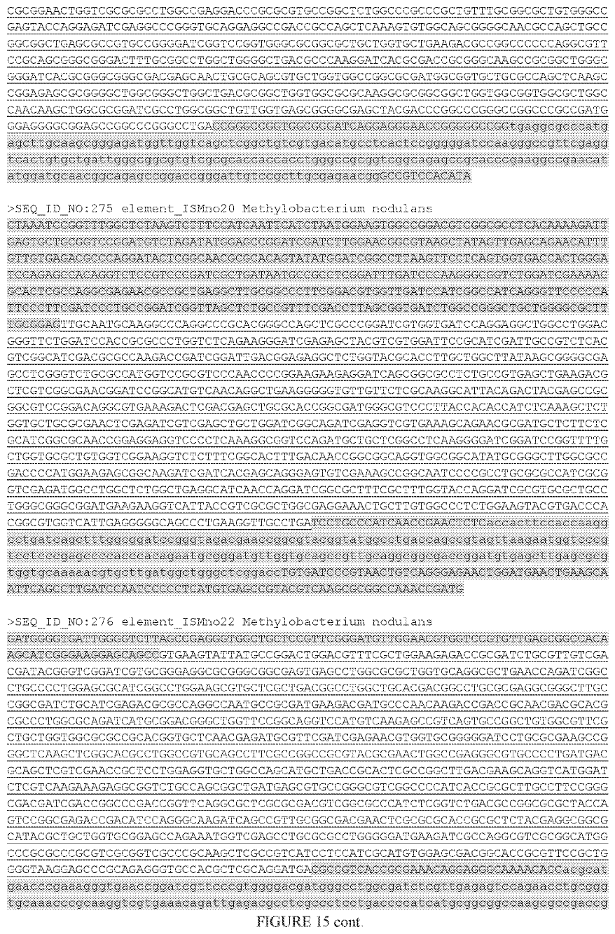

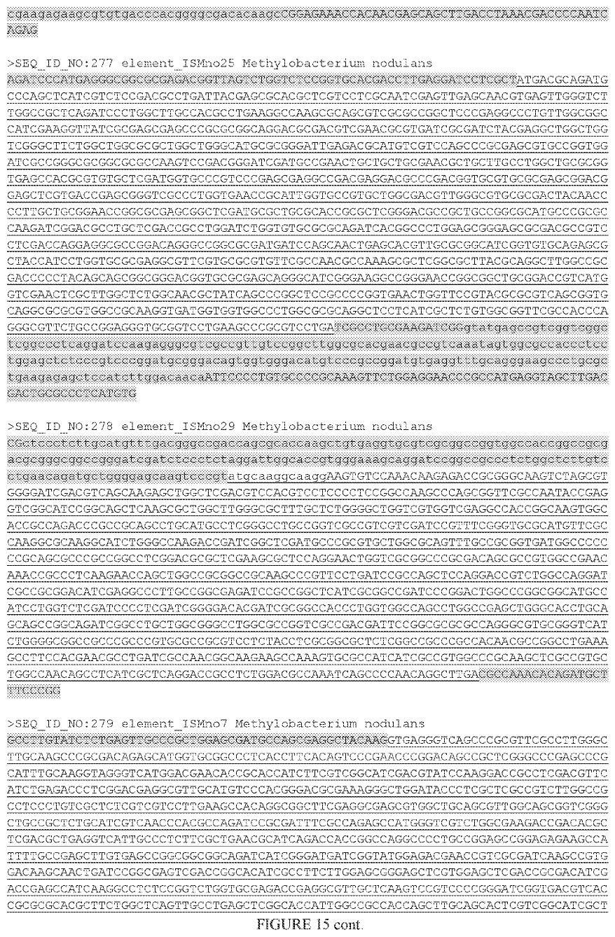

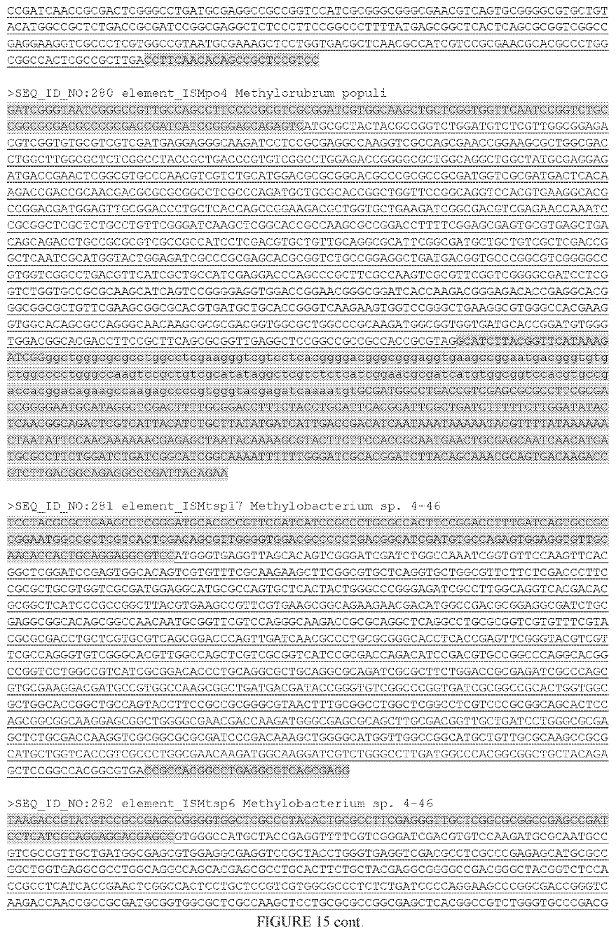

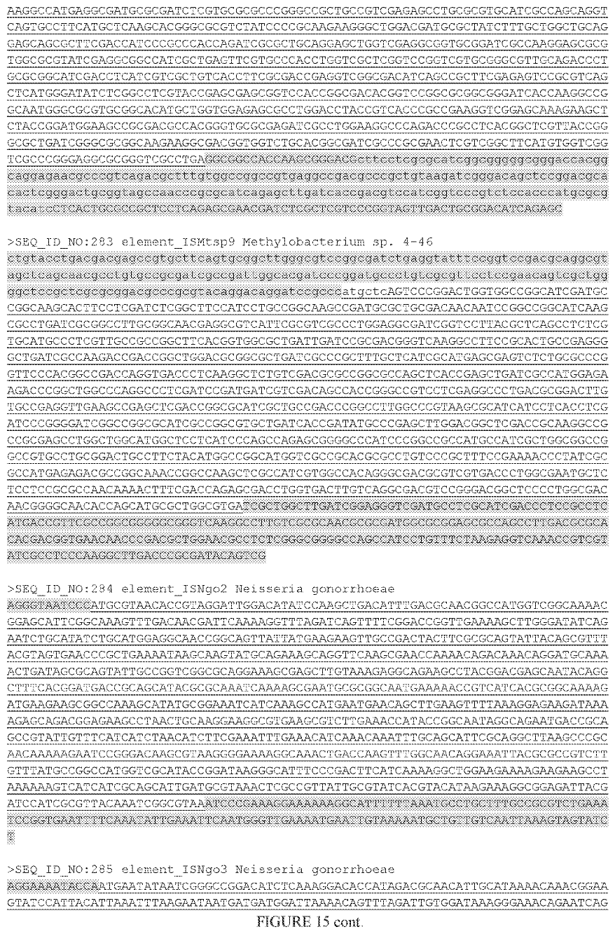

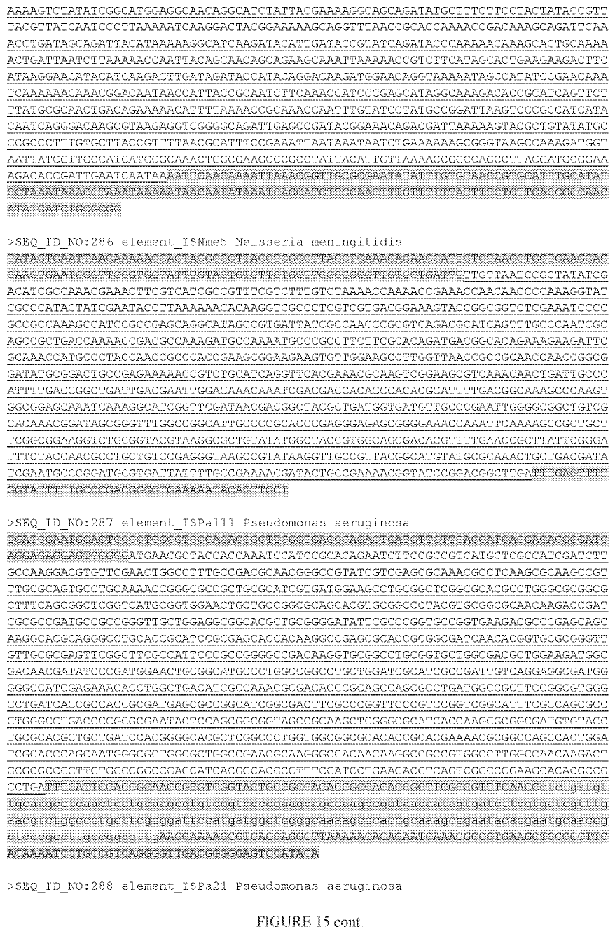

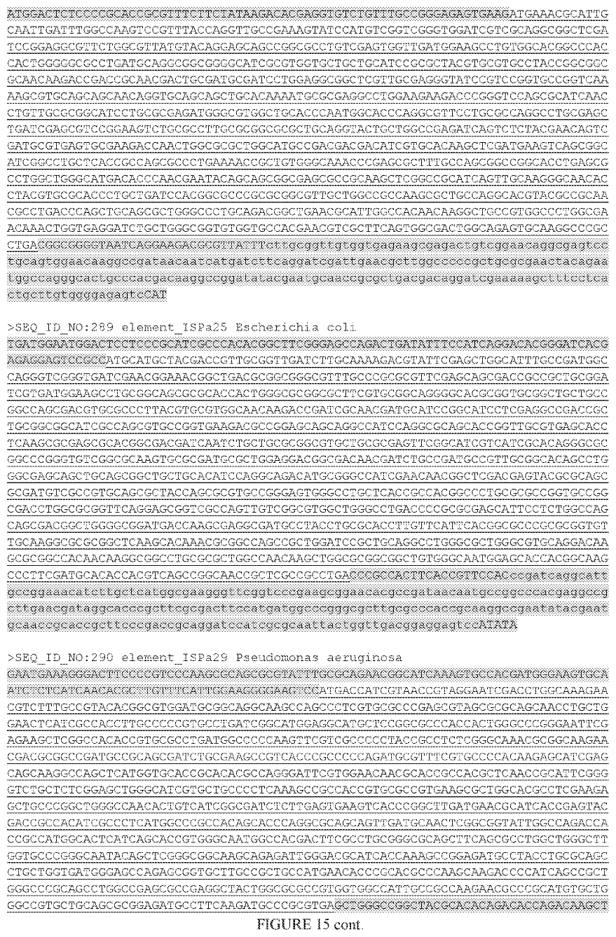

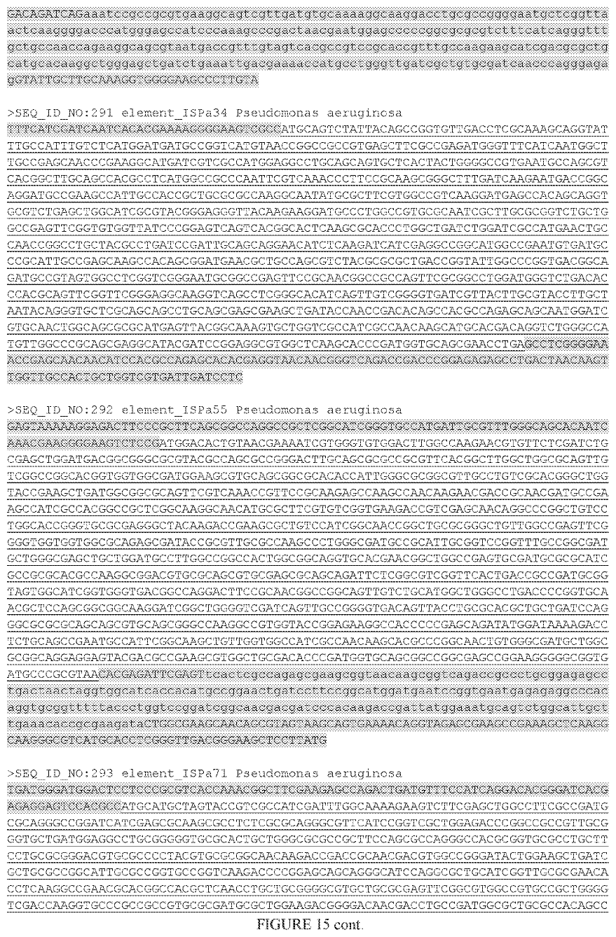

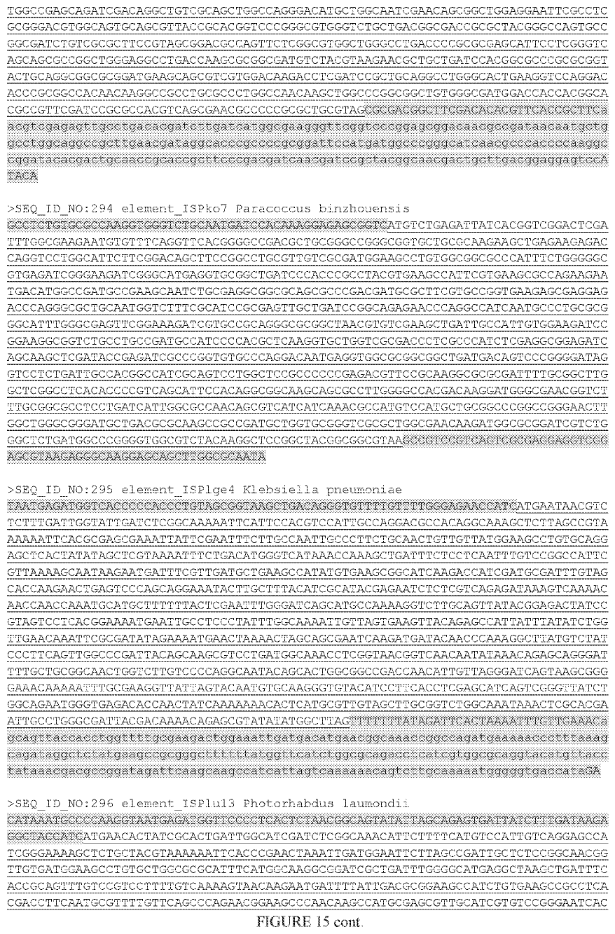

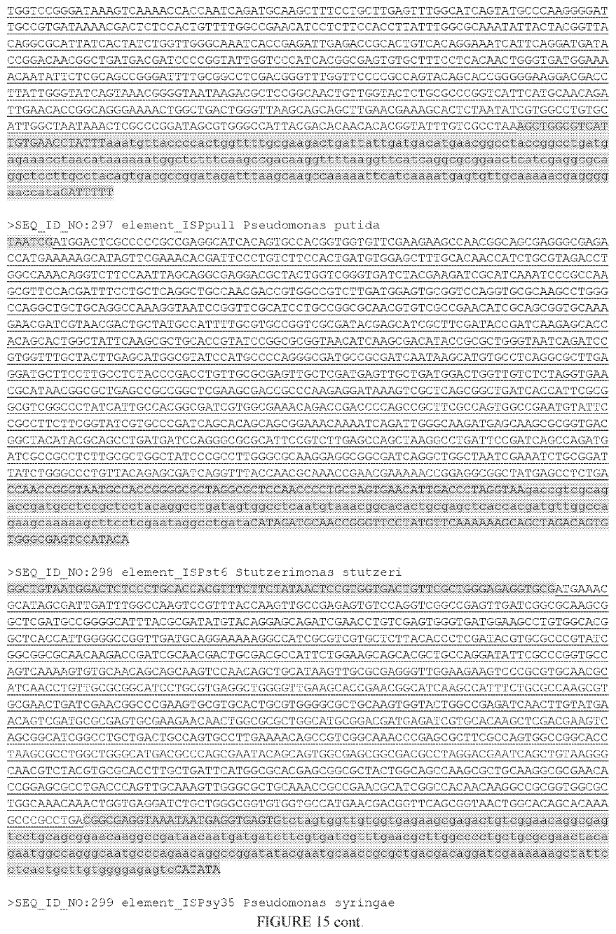

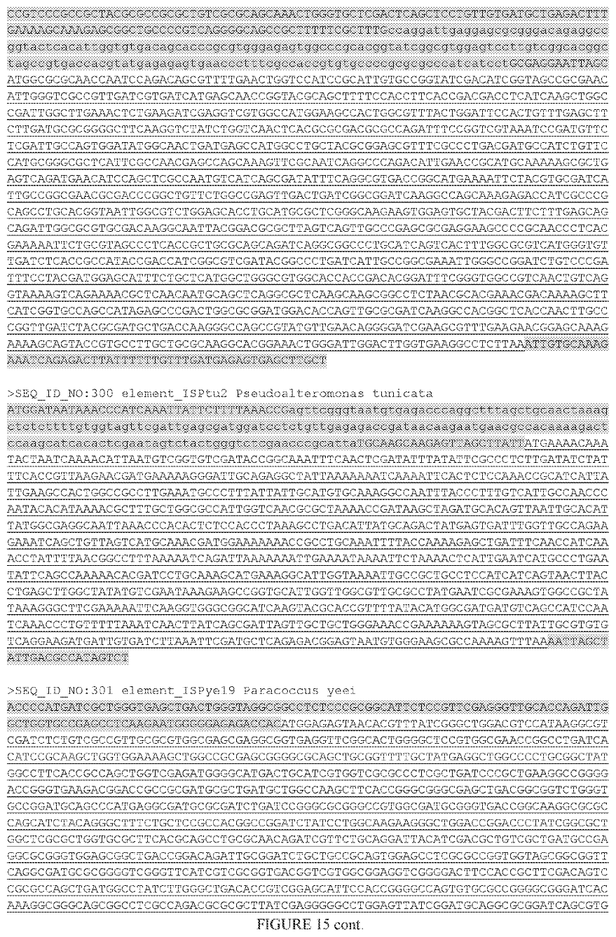

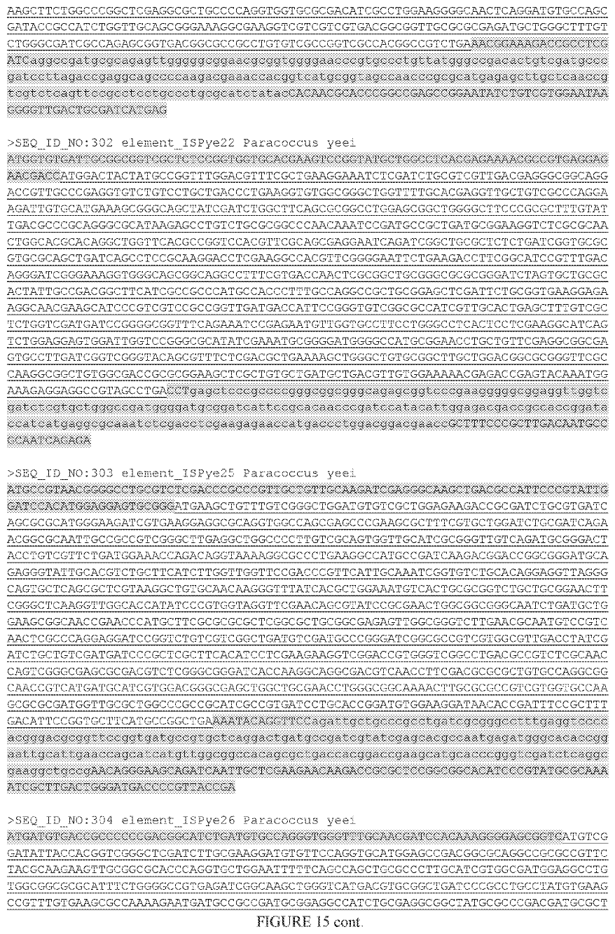

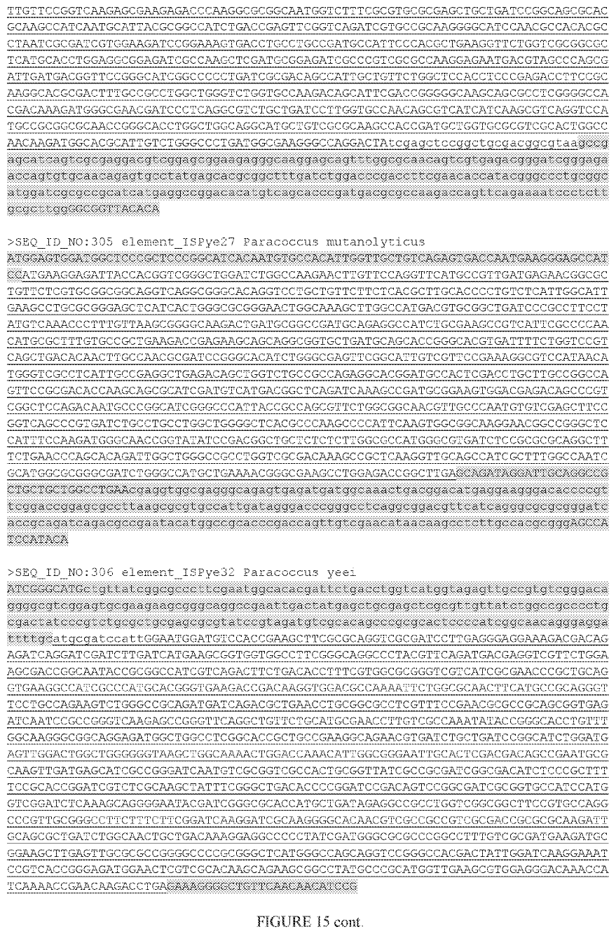

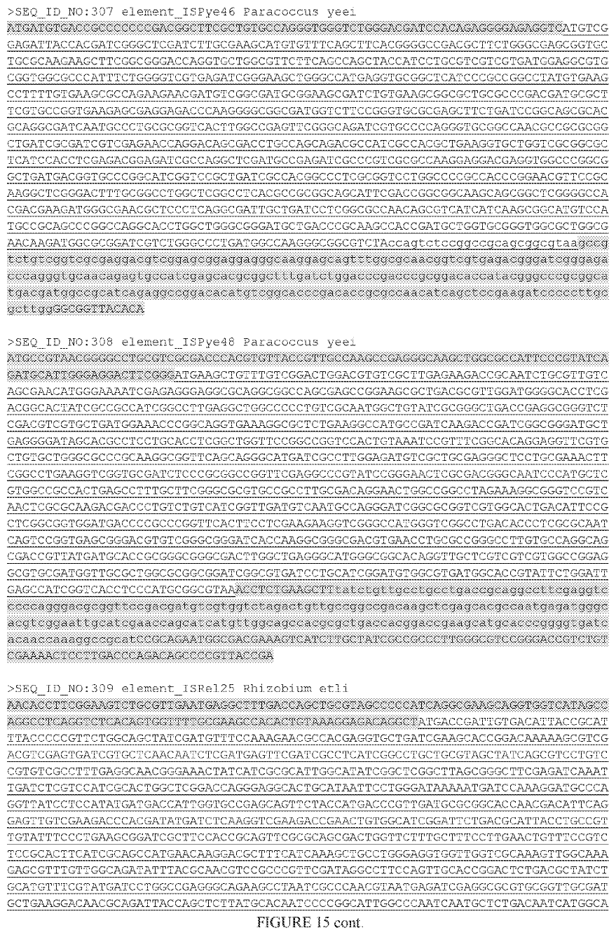

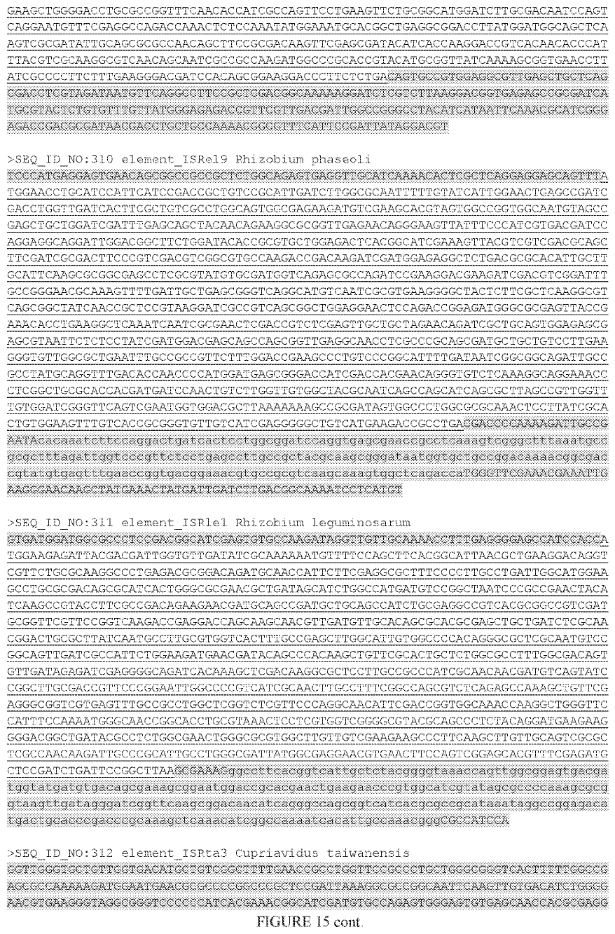

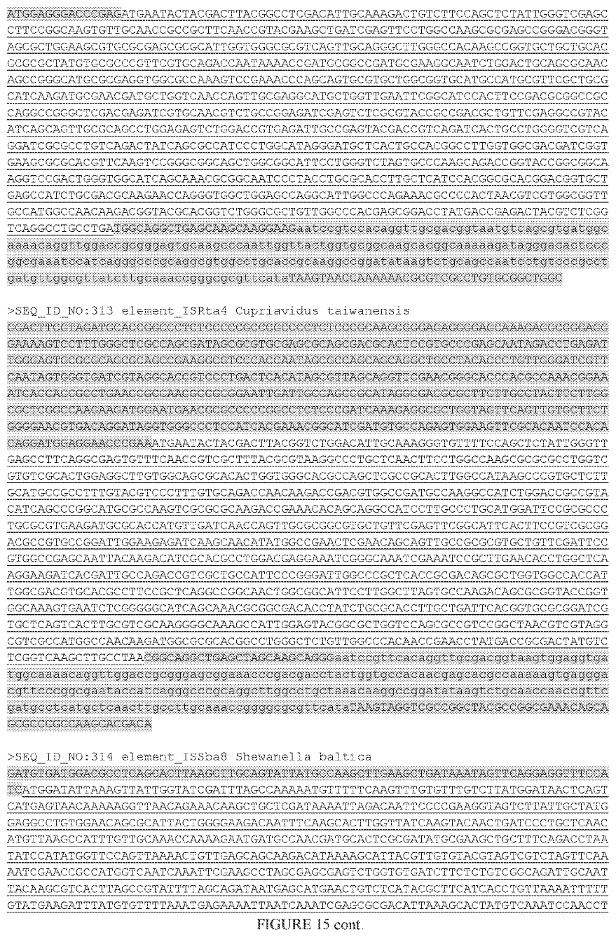

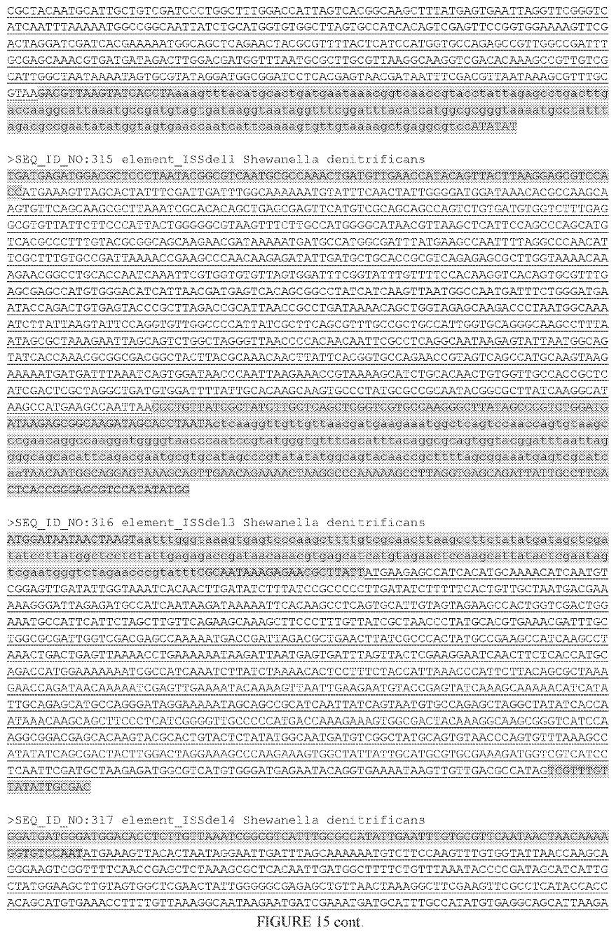

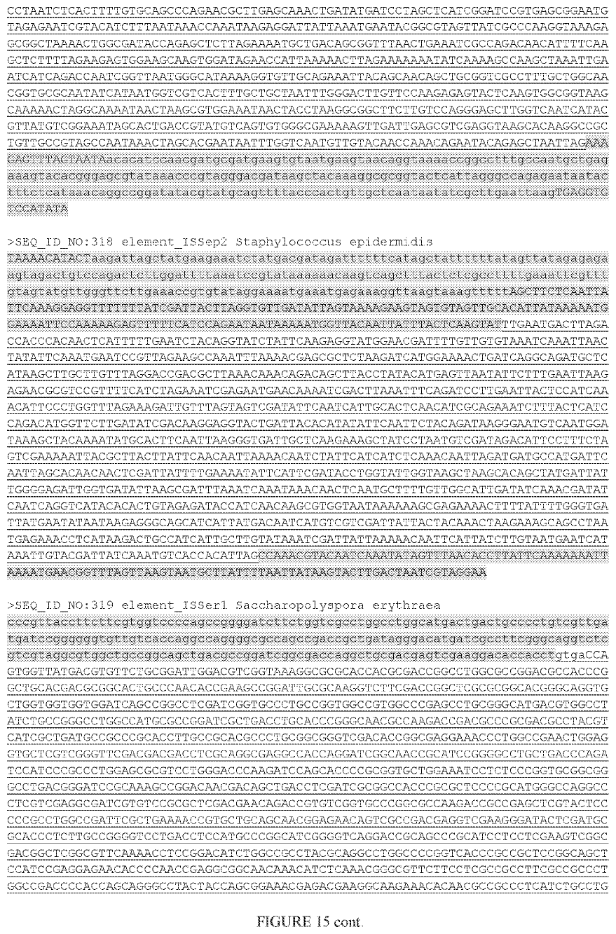

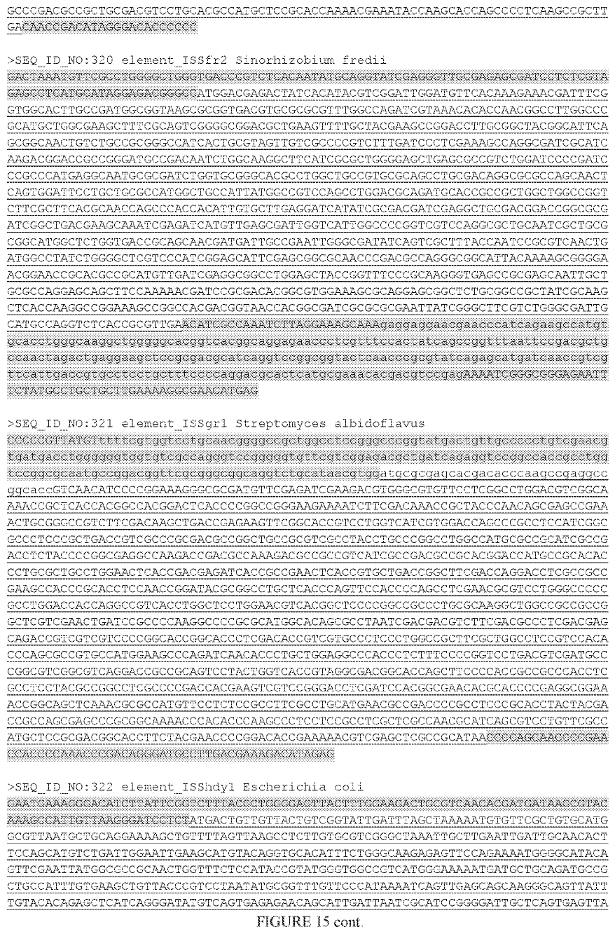

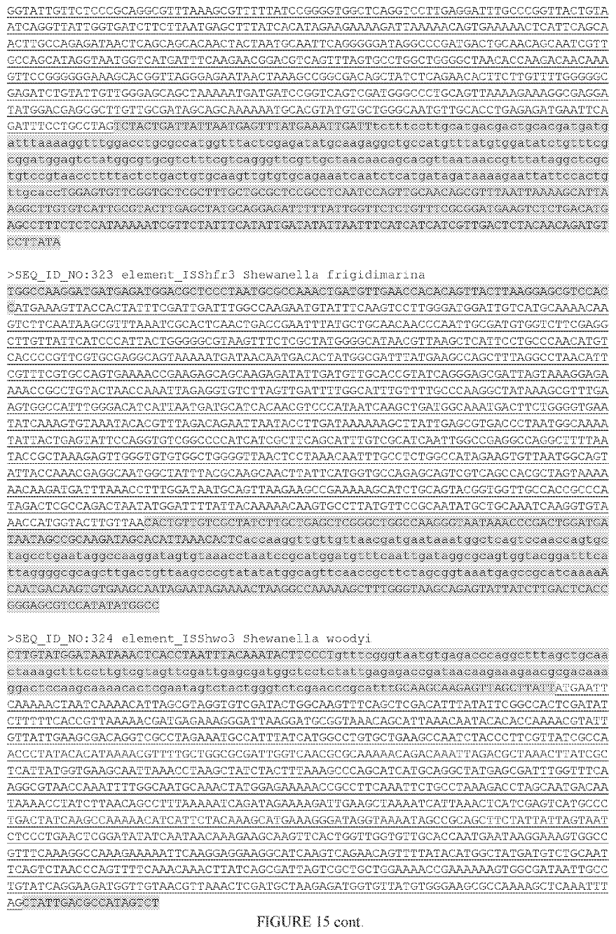

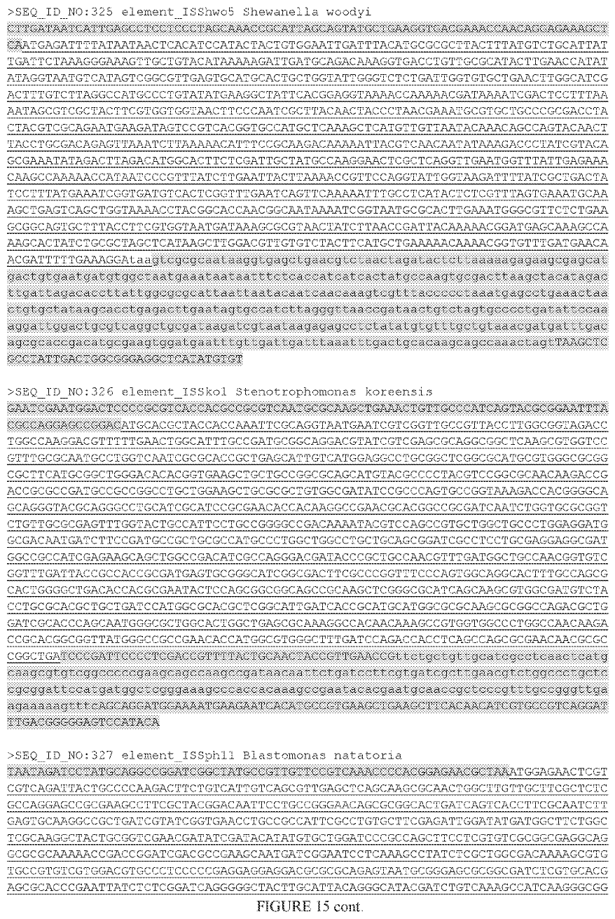

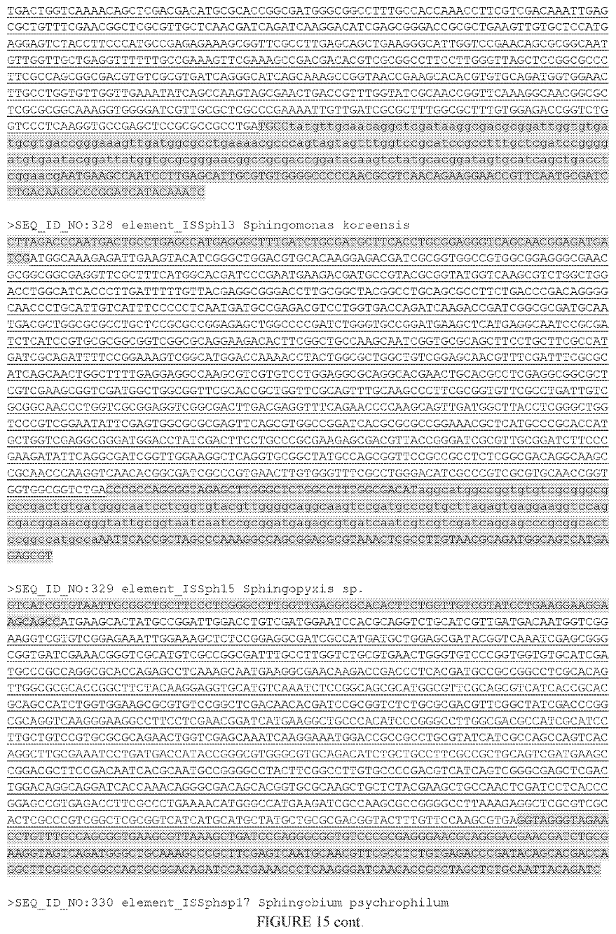

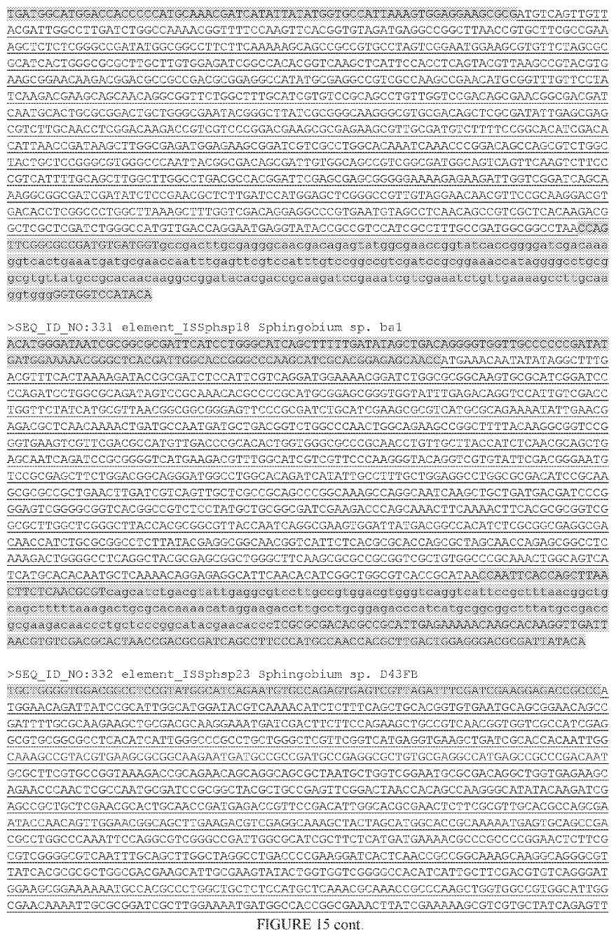

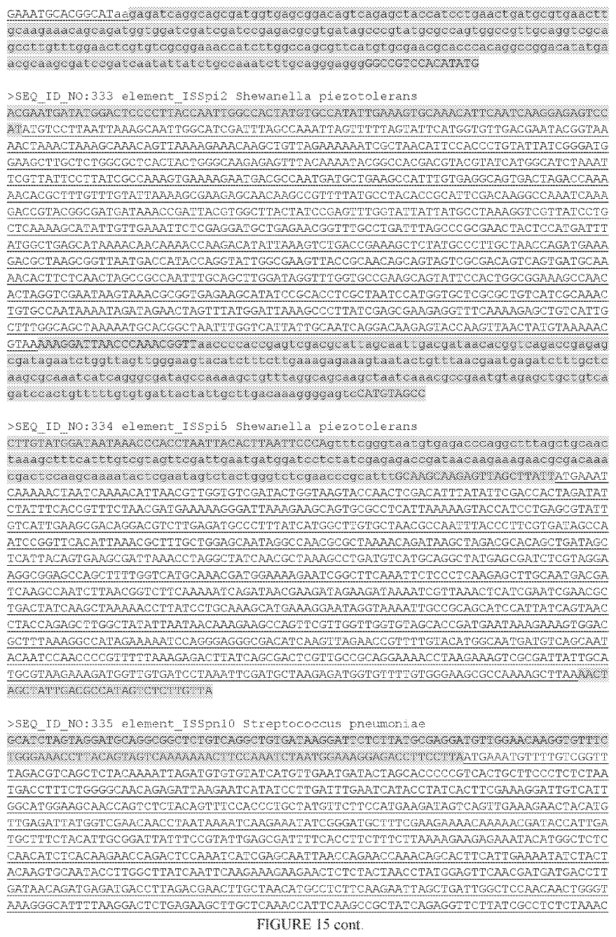

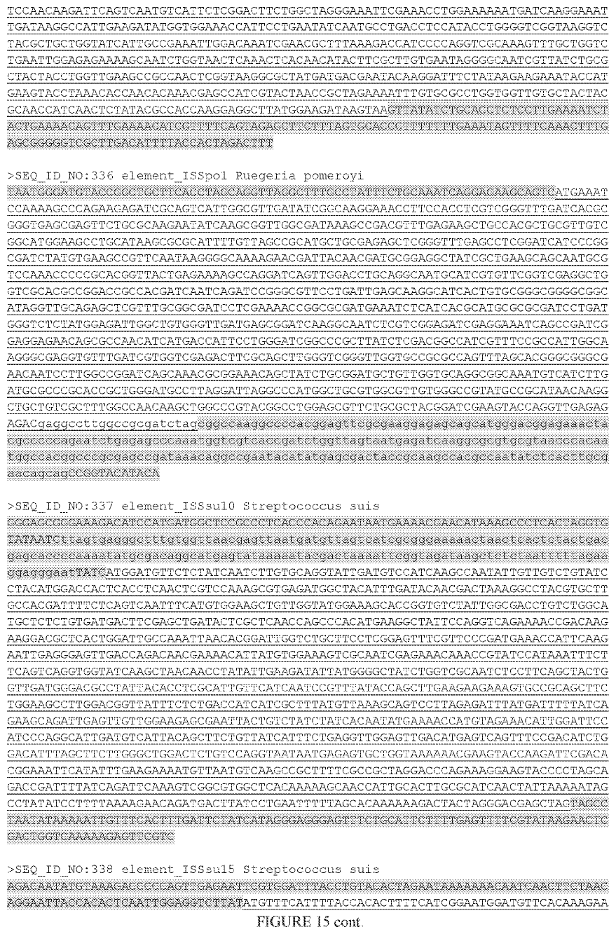

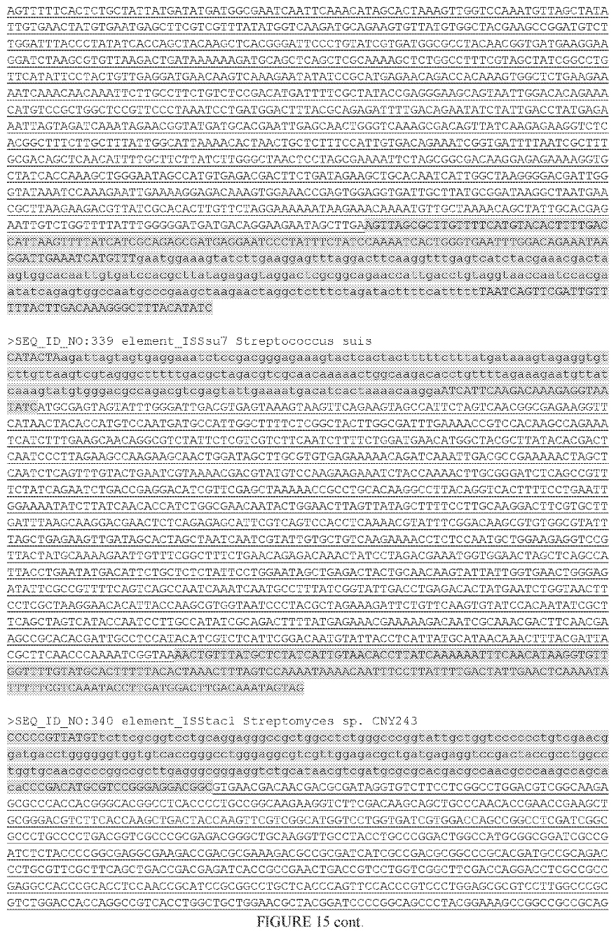

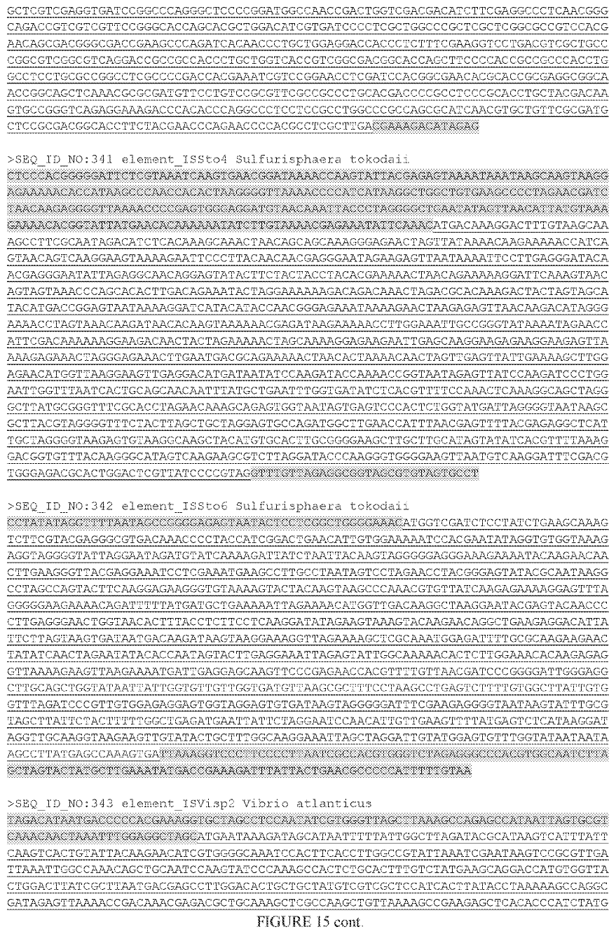

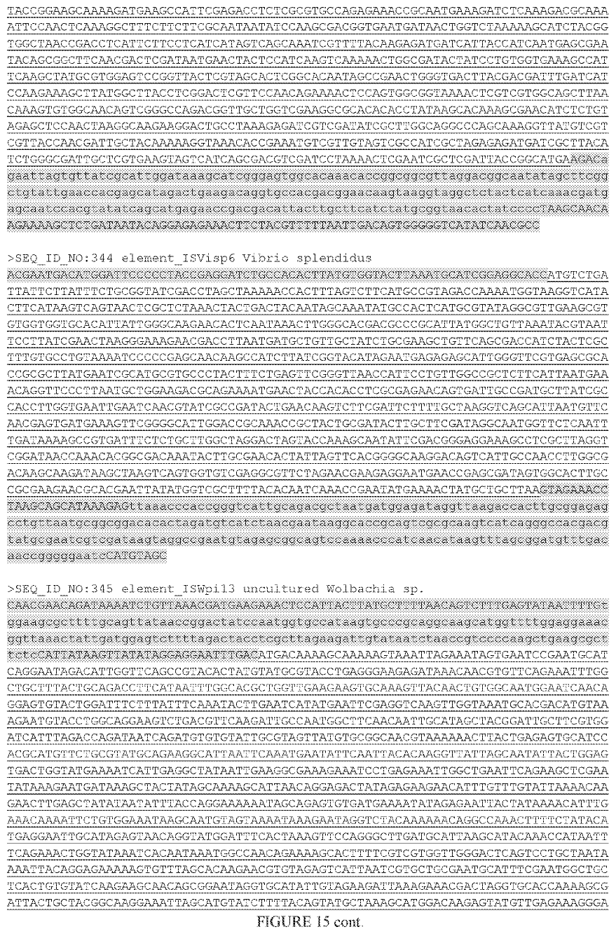

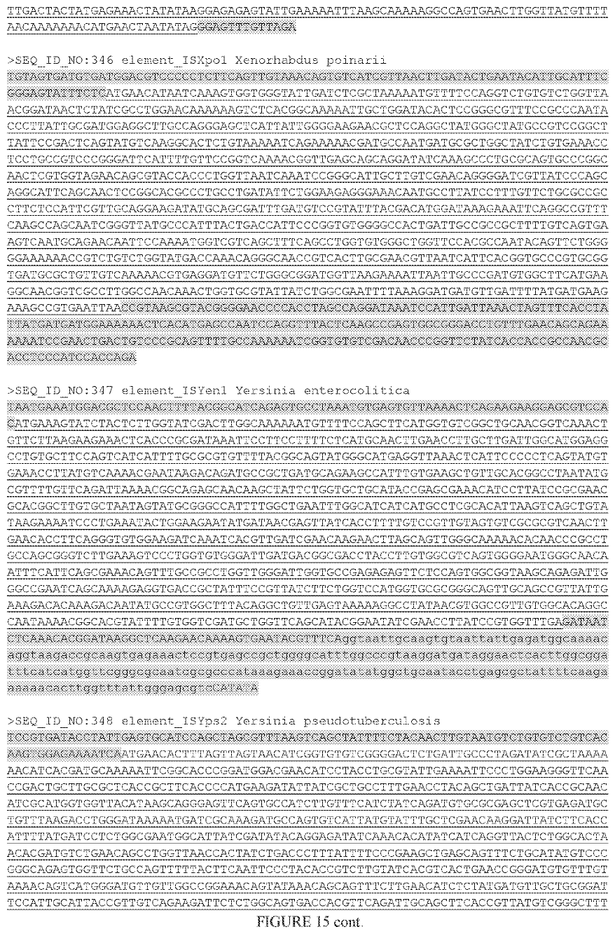

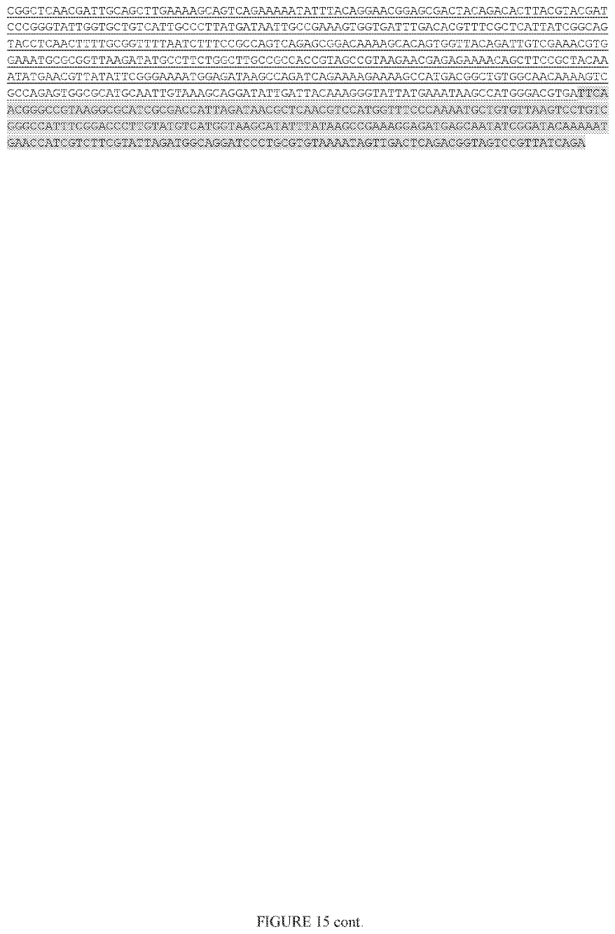

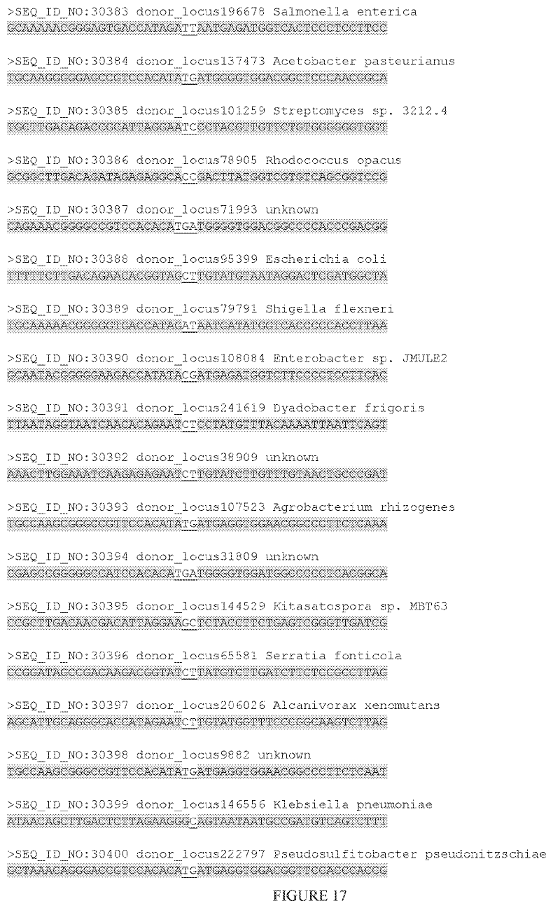

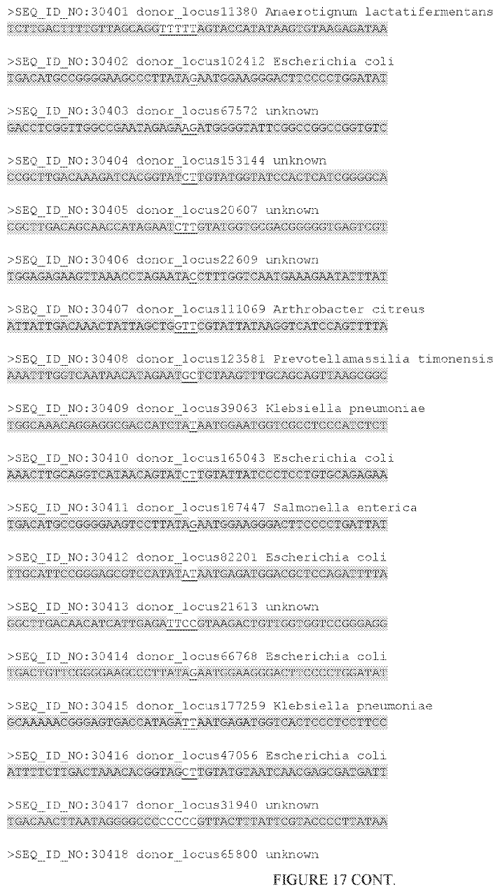

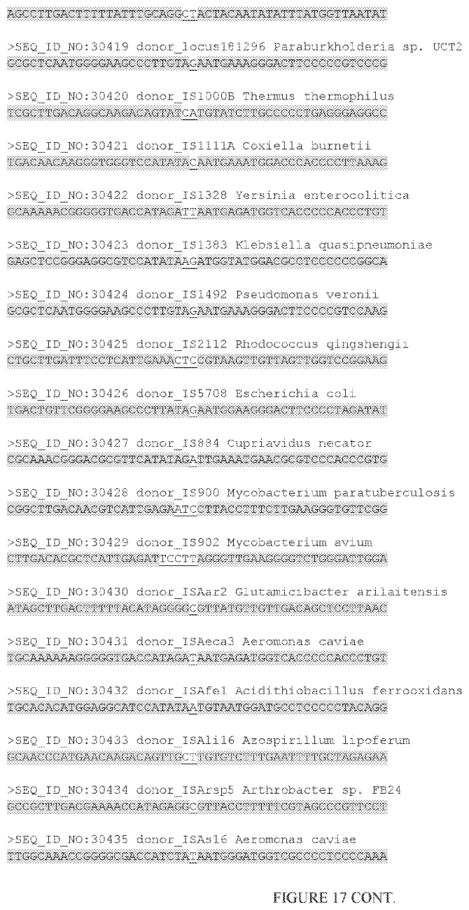

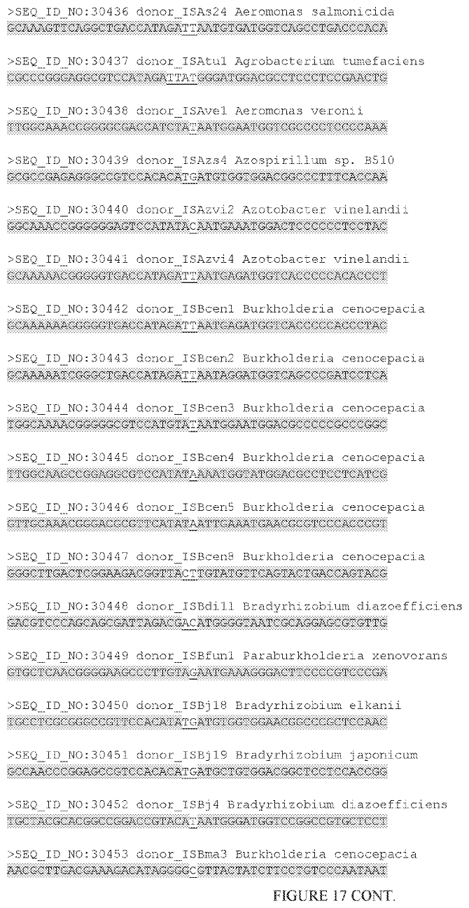

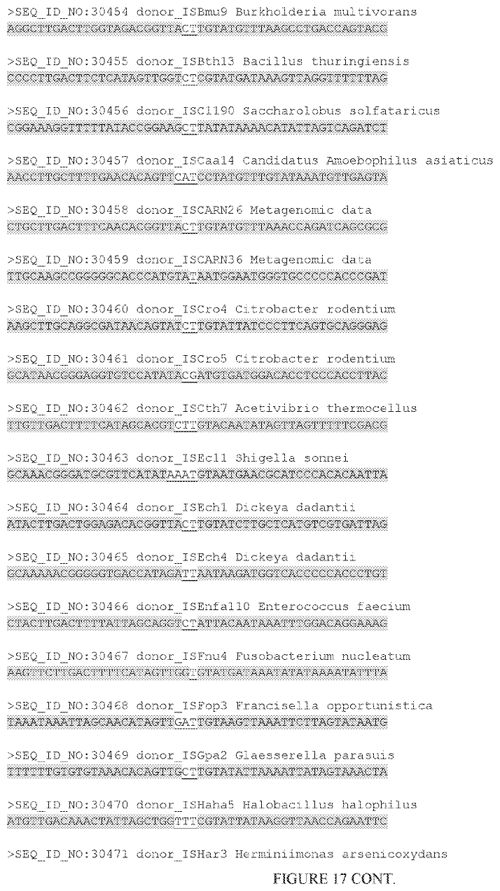

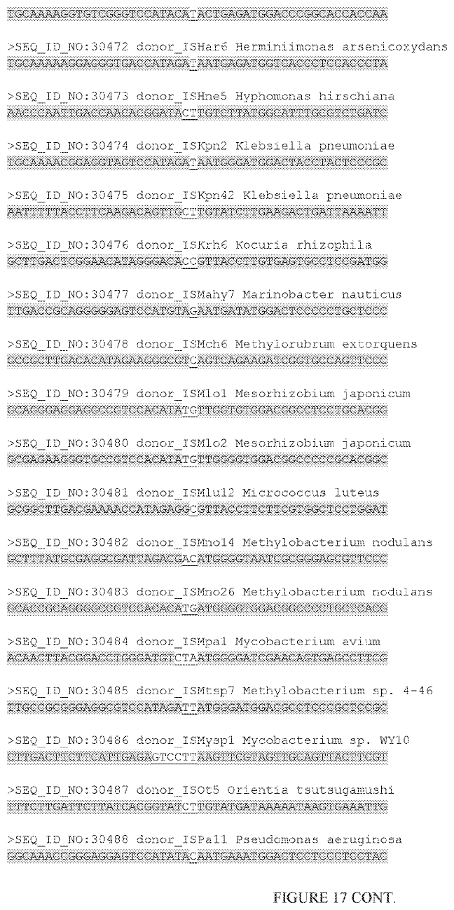

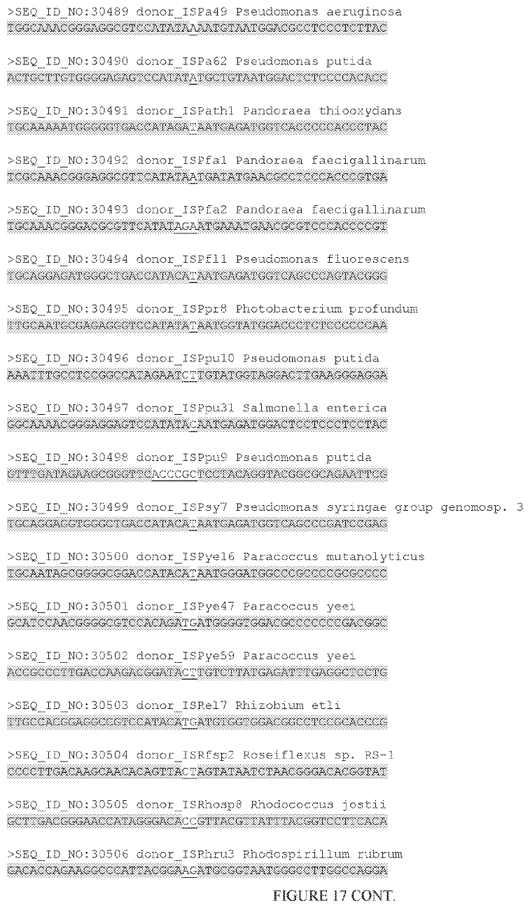

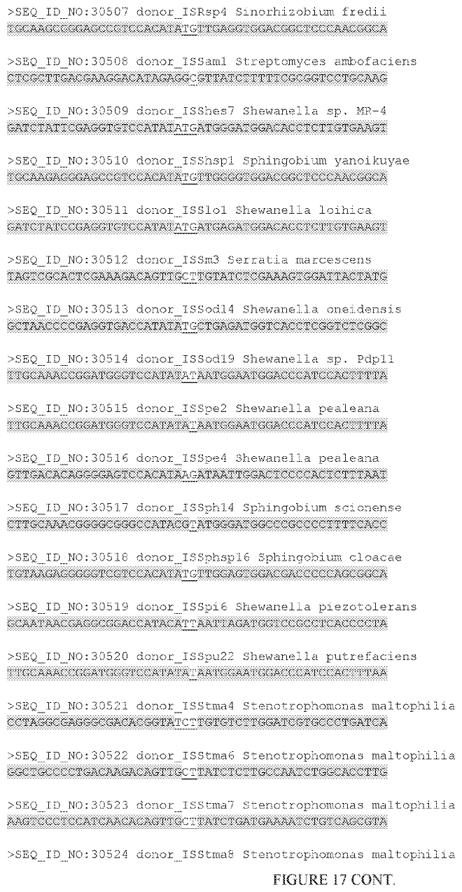

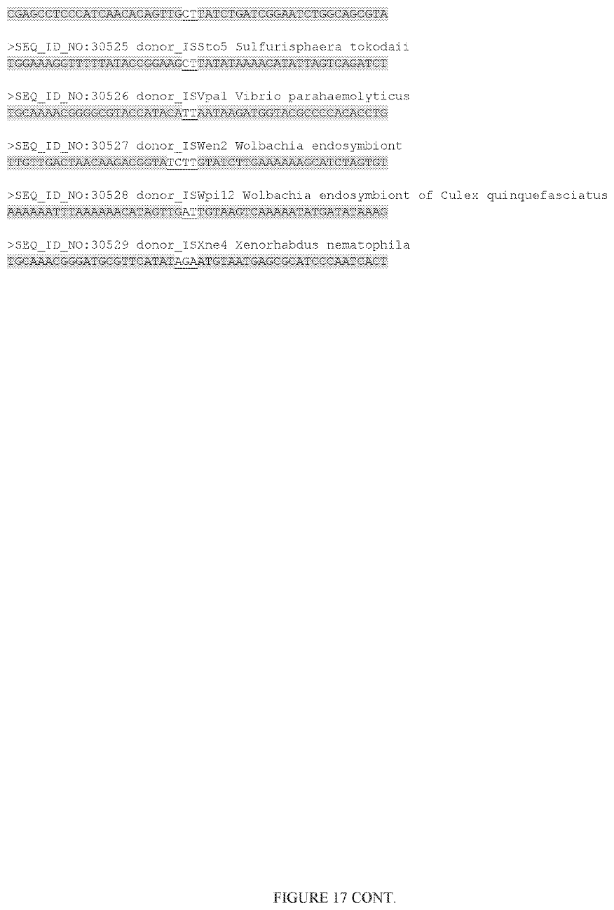

The left non-coding end (LE) of an IS110 element refers to the nucleotide sequence 5′ of the start codon of the IS110 element encoded IS110 transposase that extends (upstream) to the core or the 5′ terminal end of the element. Thus, LE is simply defined as the sequence that comes between the CDS and the core or the 5′ end of the element. The 5′ terminal end of the element may be defined using comparative (meta)genomics, or by analysis of bridgeRNA specificity for the donor sequences found at the terminus of the LE or by BLAST similarity searches to IS110s with terminal ends defined with the previous two methods. See Examples 1 and 2. In some embodiments, LE comprises an LE sequence provided in FIG. 15 (SEQ ID NOS: 1-348) or FIG. 17 (SEQ ID NOS: 30354-30529). In some embodiments, LE comprises an LE sequence provided in SEQ ID NOS: 349-10175 or 30530-40356.

The right non-coding end (RE) of an IS110 element refers to the nucleotide sequence 3′ of the stop codon of the IS110 element encoded IS110 transposase that extends (downstream) to the core or the 3′ terminal end of the element. Thus, RE is simply defined as the sequence that comes between the CDS and the core or the 3′ end of the element. The 3′ terminal end of the element may be defined using comparative (meta)genomics or by analysis of bridgeRNA specificity for the donor sequences found at the terminus of the RE or by BLAST similarity searches to IS110s with terminal ends defined with the previous two methods. See Examples 1 and 2. In some embodiments, RE comprises an RE sequence provided in FIG. 15 (SEQ ID NOS: 1-348) or FIG. 17 (SEQ ID NOS: 30354-30529). In some embodiments, RE comprises an RE sequence provided in SEQ ID NOS: 349-10175 or 30530-40356.

For IS110 transposons that comprise a core sequence, the core refers to an identical nucleotide sequence found immediately 5′ and 3′ of the left non-coding end (LE) and right non-coding end (RE), respectively. The core was previously referred to as “target intervening core” or “TIC” and any references to target intervening core or TIC refer to the core sequence. In some embodiments, the core sequence is 1-10 nucleotides long. In some embodiments, the core sequence is 1-5 nucleotides long. In some embodiments, the core sequence is 1 nucleotide long, 2 nucleotides long, 3 nucleotides long, 4 nucleotides long, 5 nucleotides long, 6 nucleotides long, 7 nucleotides long, 8 nucleotides long, 9 nucleotides long, or 10 nucleotides long. In certain embodiments, the core sequence is 2 nucleotides long. In some embodiments, a core comprises a core sequence provided in FIG. 15 (SEQ ID NOS: 1-348) or FIG. 17 (SEQ ID NOS: 30354-30529). In some embodiments, a core comprises a core sequence provided in SEQ ID NOS: 349-10175 or 30530-40356.

Exemplary IS110 family IS element sequences are provided in FIG. 15 (SEQ ID NOS: 1-348). The nucleotide sequences of LE, core (where present), the transposase, and RE are indicated as described above for FIG. 15. Additional exemplary IS110 family IS element sequences are provided in SEQ ID NOS: 349-10175. The nucleotide sequences of LE, core (where present), the transposase CDS, RE, and bridgeRNA are indicated as features of the sequence listing.

For IS110 elements that comprise a core sequence, RE-core-LE refers to a concatenation of the nucleotide sequences of the RE, core, and LE which a portion thereof (e.g., the donor site sequence comprised of LD-core-RD) may be bound by an IS110 family transposase described herein (e.g., see Section C). In some embodiments, RE-core-LE comprises an LE, core, and RE provided in FIG. 15 (SEQ ID NOS: 1-348) or FIG. 17 (SEQ ID NOS: 30354-30529). In some embodiments, RE-core-LE comprises an LE, core, and RE provided in SEQ ID NOS: 349-10175 or 30530-40356. The nucleotide sequences of LE, core (where present), and RE are indicated as features of the sequence listing. In some embodiments, LD-core-RD comprises an LD, core, and RD provided in FIG. 17 (SEQ ID NOS: 30354-30529). The nucleotide sequences of LD and RD are indicated in bold and the nucleotide sequence of core sequence is represented as non-highlighted text with a single underline. In some embodiments, LD-core-RD comprises an LD, core, and RD derived from the LDG and RDG provided in FIG. 15 (SEQ ID NOS: 1-348).

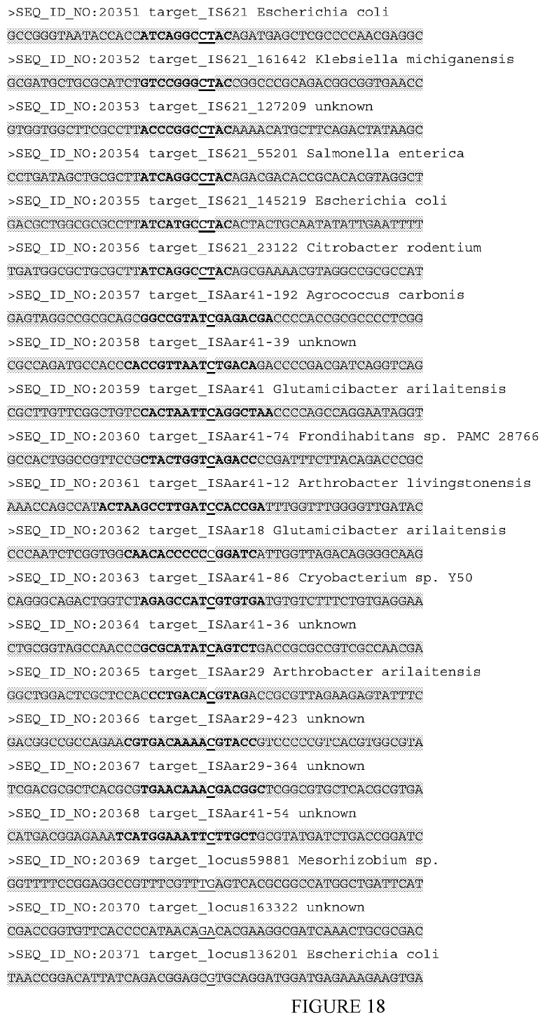

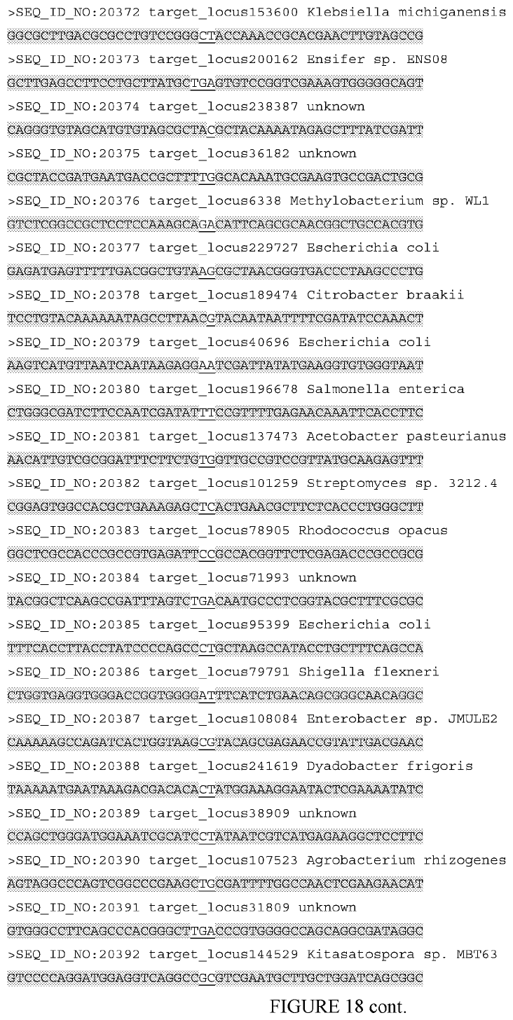

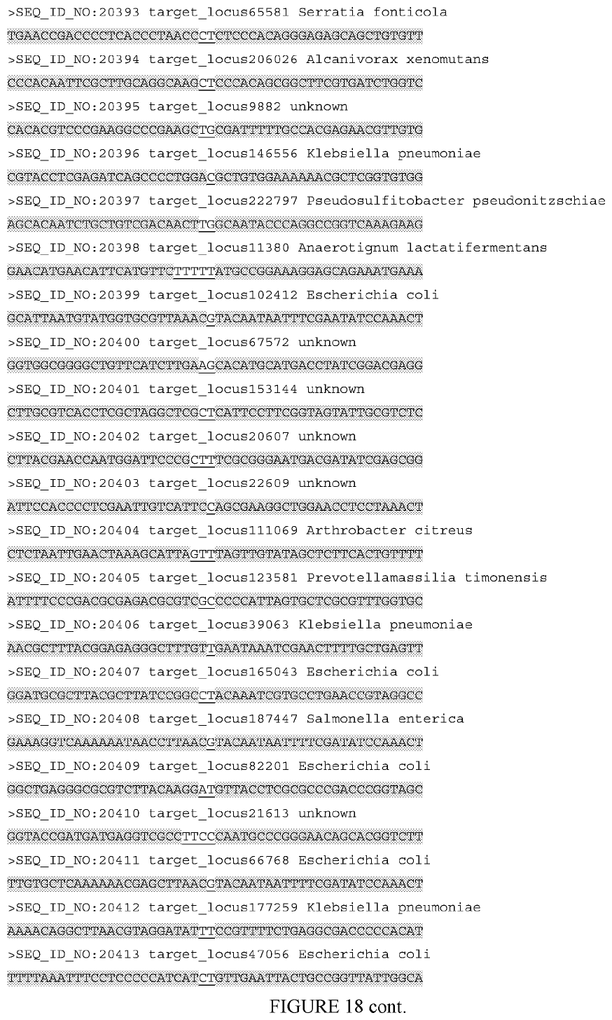

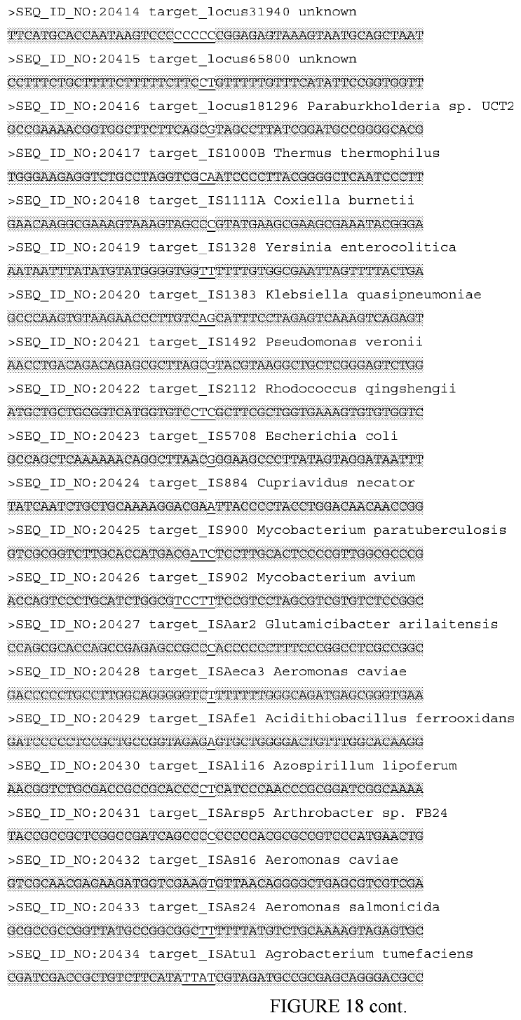

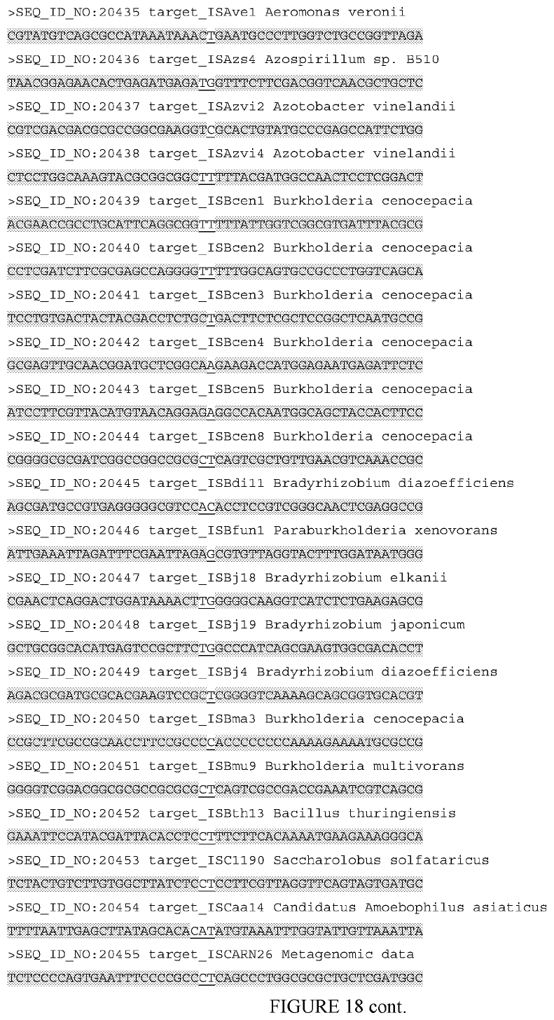

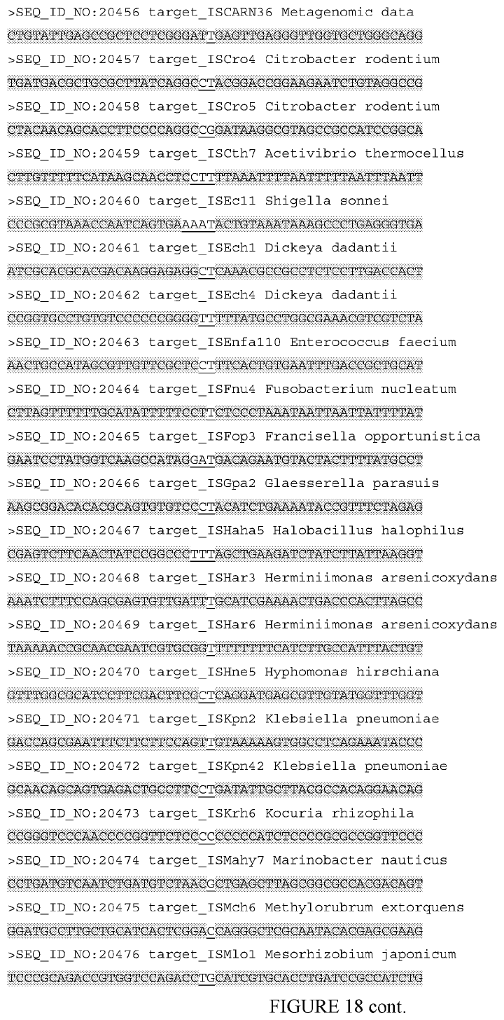

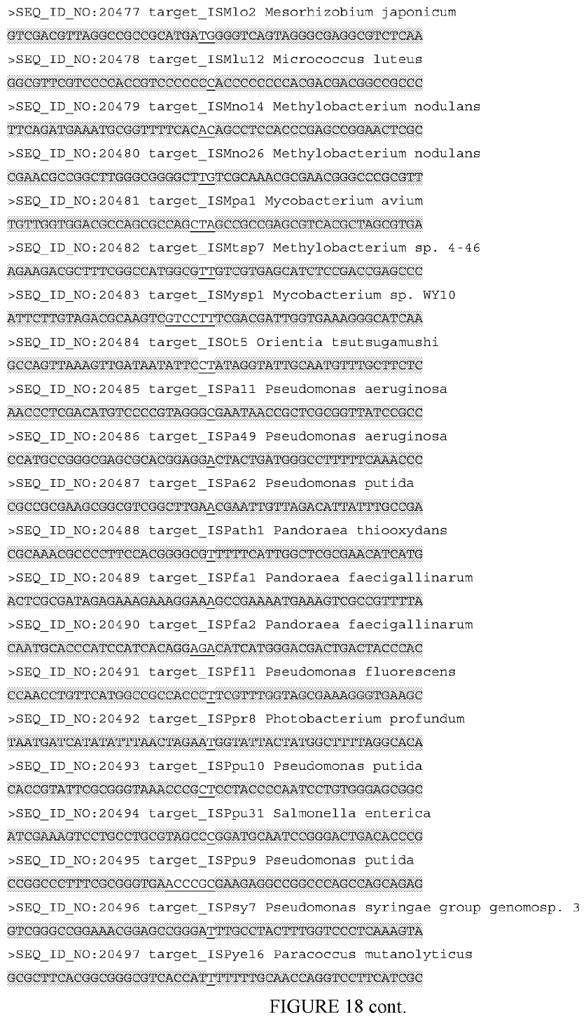

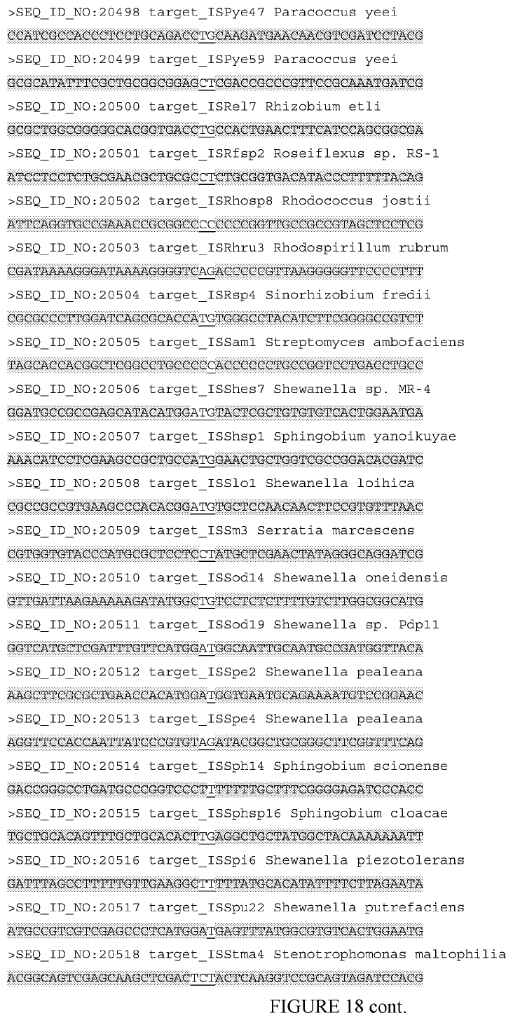

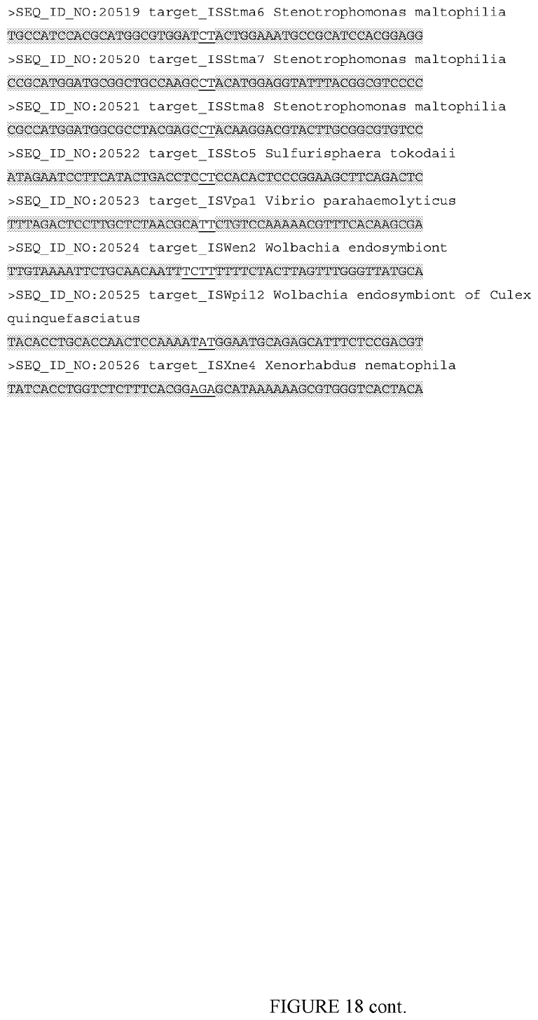

For IS110 elements that comprise a core sequence, LF-core-RF refers to a concatenation of the nucleotide sequences of the LF, core, and RF which a portion thereof (e.g., the target site sequence comprised of LT-core-RT) may be bound by an IS110 family transposase described herein (e.g., see Section C). In some embodiments, LF-core-RF comprises a LF, core, and RF provided in FIG. 18 (SEQ ID NOs: 20351-20526). In some embodiments, RF-core-LF comprises an LF, core, and RF provided in SEQ ID NOs: 20527-30353. The nucleotide sequences of LF, core (where present), and RF are indicated as features of the sequence listing. In some embodiments, LT-core-RT comprises an LT, core, and RT provided in FIG. 18 (SEQ ID NOS: 20351-20368). The nucleotide sequences of LT and RT are indicated in bold and the nucleotide sequence of core sequence is represented as non-highlighted text with a single underline. In some embodiments, LT-core-RT comprises an LT, core, and RT derived from the LTG and RTG provided in FIG. 15 (SEQ ID NOS: 1-348).

For IS110 elements that do not comprise a core sequence, RE-LE refers to a concatenation of the nucleotide sequences of the RE and LE which a portion thereof (e.g., the donor site sequence comprises of LD-RD) may be bound by an IS110 family transposase described herein (e.g., see Section C). In some embodiments, RE-LE comprises an LE and RE provided in FIG. 15 (SEQ ID NOS: 1-348) or FIG. 17 (SEQ ID NOS: 30354-30529). In some embodiments, RE-LE comprises an LE and RE provided in SEQ ID NOS: 349-10175 or 30530-40356. In some embodiments, LD-RD comprises an LD and RD derived from the LDG and RDG provided in FIG. 15 (SEQ ID NOS: 1-348).

For IS110 elements that do not comprise a core sequence, LF-RF refers to a concatenation of the nucleotide sequences of the LF and RF which a portion thereof (e.g., the target site sequence comprised of LT-RT) may be bound by an IS110 family transposase described herein (e.g., see Section C). In some embodiments, LF-RF comprises an LF and RF provided in FIG. 18 (SEQ ID NOs: 20351-20526). In some embodiments, LF-RF comprises a LF and RF provided in SEQ ID NOs: 20527-30353. In some embodiments, LT-RT comprises an LT and RT derived from the LTG and RTG provided in FIG. 15 (SEQ ID NOS: 1-348).

### C. IS110 Family Transposases

The IS110 family of transposases encoded within the IS110 transposons were identified by homology searching for a DEDD catalytic domain, which is a RuvC-like domain. See Example 1. IS110 family transposases described herein comprise an N-terminal RuvC-like DEDD catalytic domain and a C-terminal transposase domain with two canonical Pfam domains as depicted in FIG. 1B. In some embodiments, the polypeptide sequence between the N-terminal RuvC-like DEDD catalytic domain and the C-terminal transposase domain comprises a linker domain comprising a coiled-coil.

Within the IS110 family, transposons can be classed into the IS110 group which are any insertion sequence (IS) element encoding an IS110 transposase and comprising a longer 5′ non-coding end (LE) than 3′ non-coding end (RE). See FIGS. 1A, D, E.

Within the IS110 family, transposons can be classed into the IS1111 group which are any insertion sequence (IS) element encoding an IS110 transposase and typically comprising a longer 3′ non-coding end (RE) than 5′ non-coding end (LE). See FIGS. 1A, D, E.

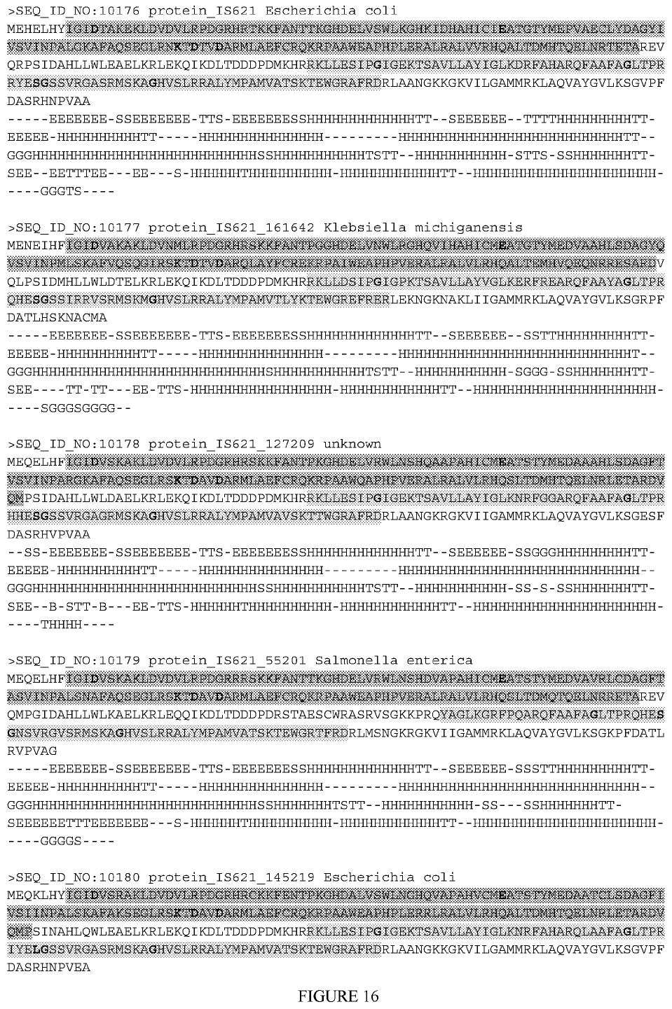

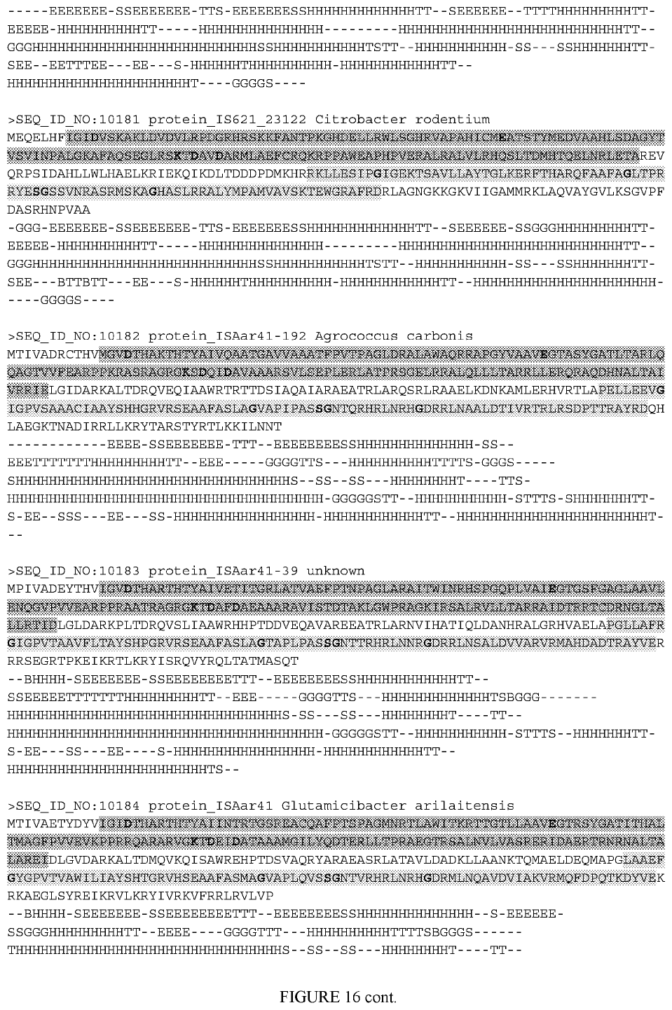

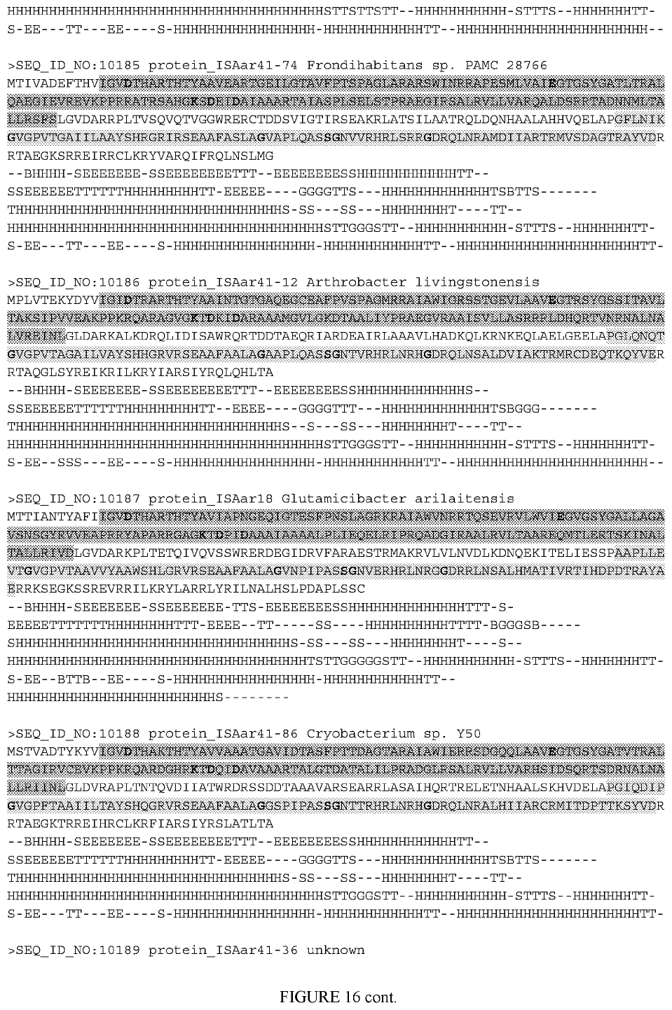

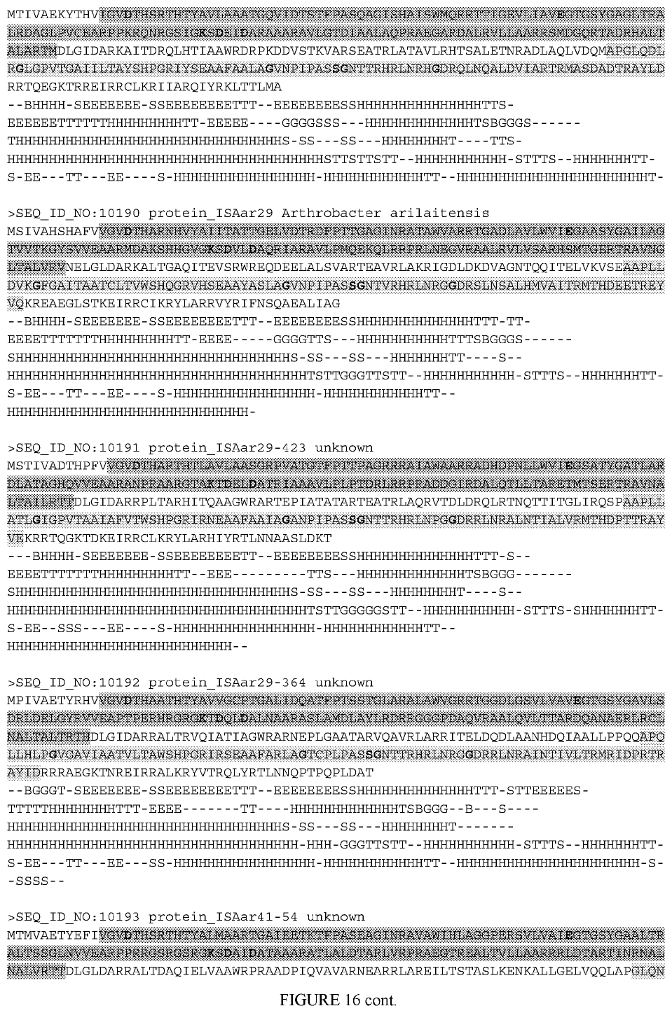

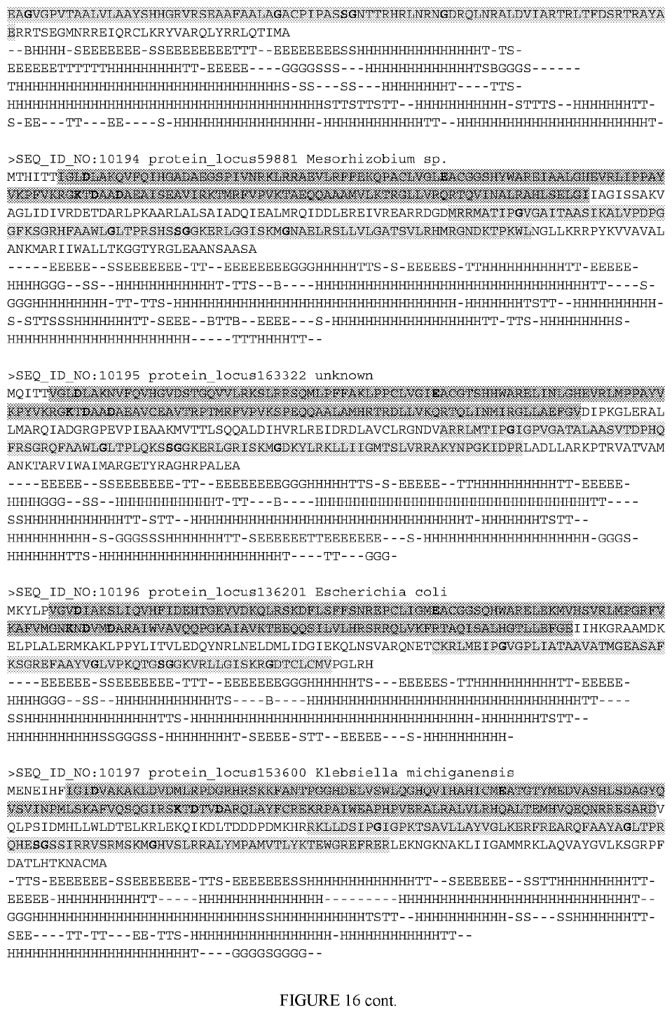

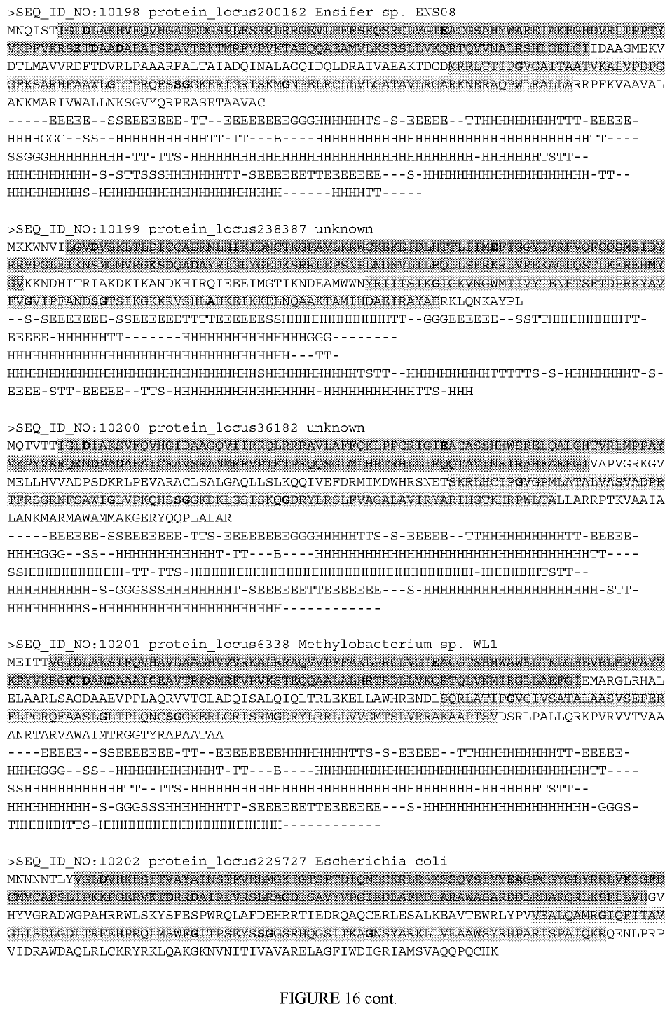

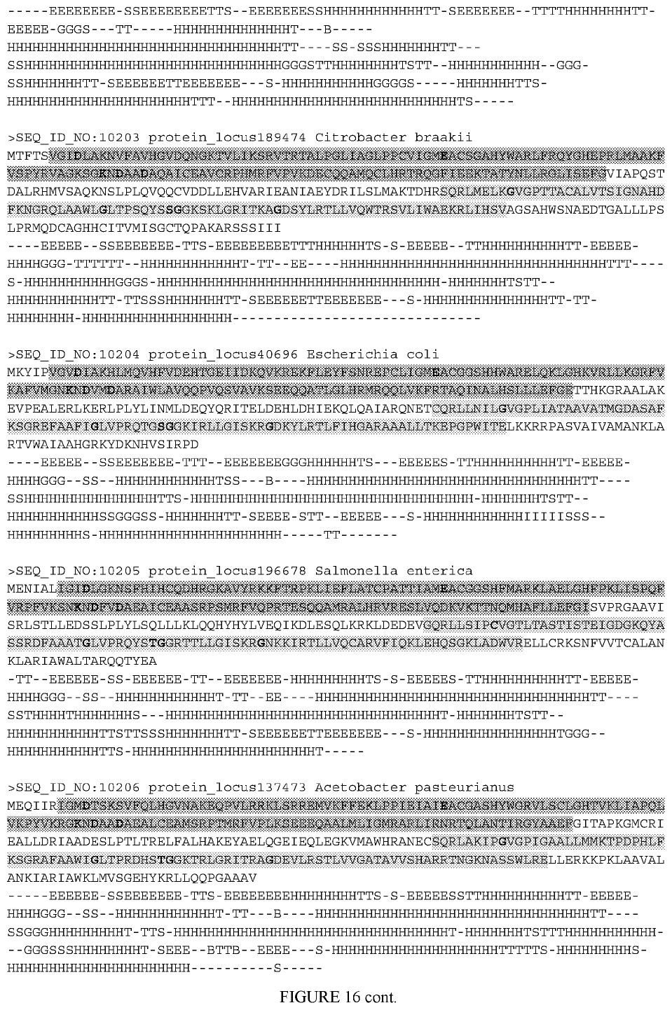

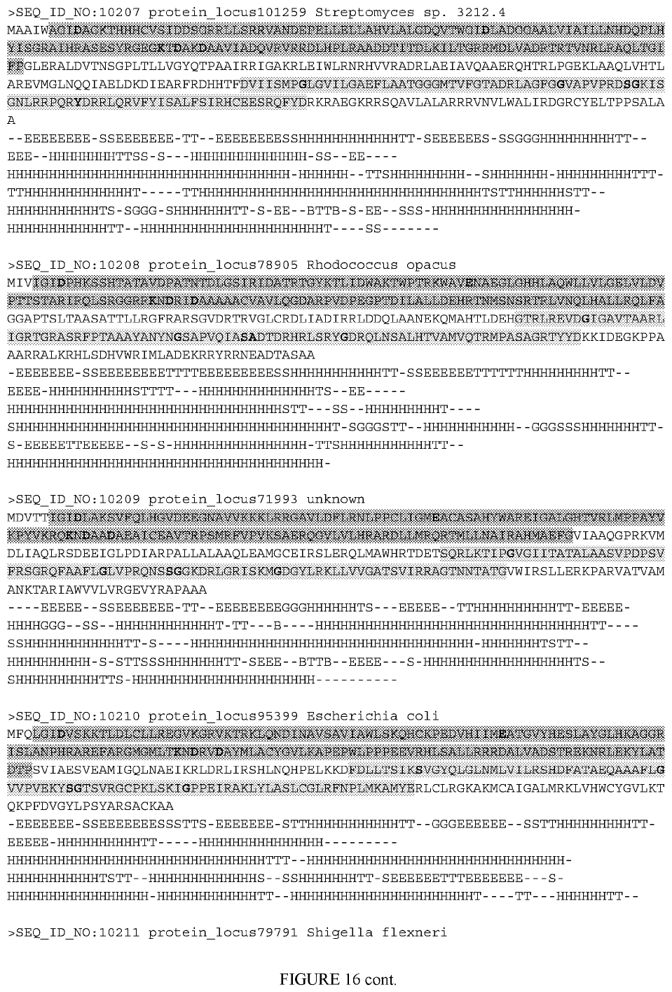

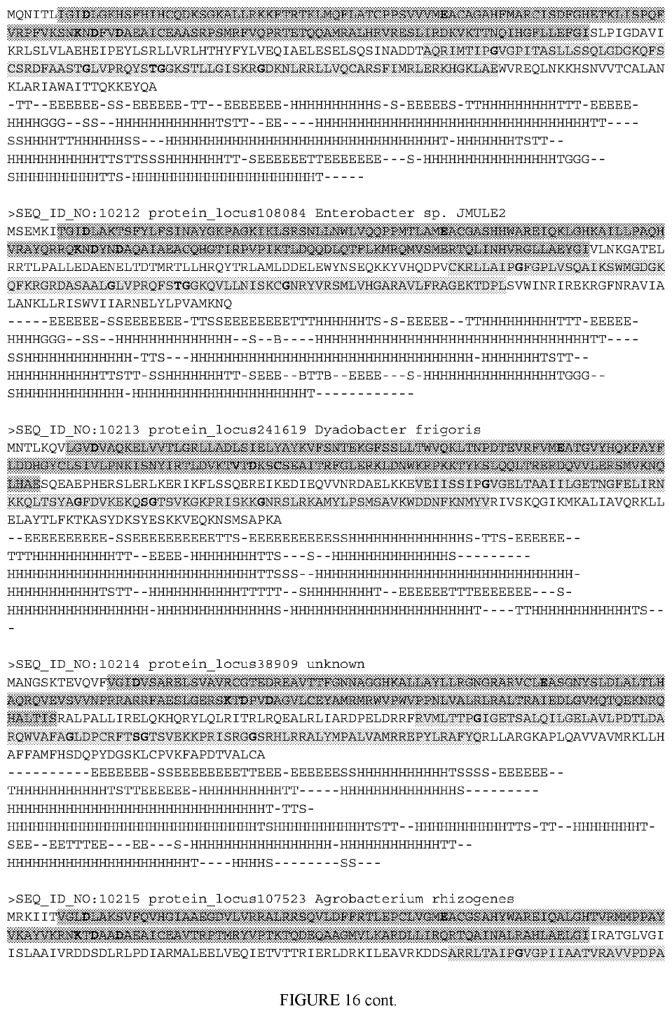

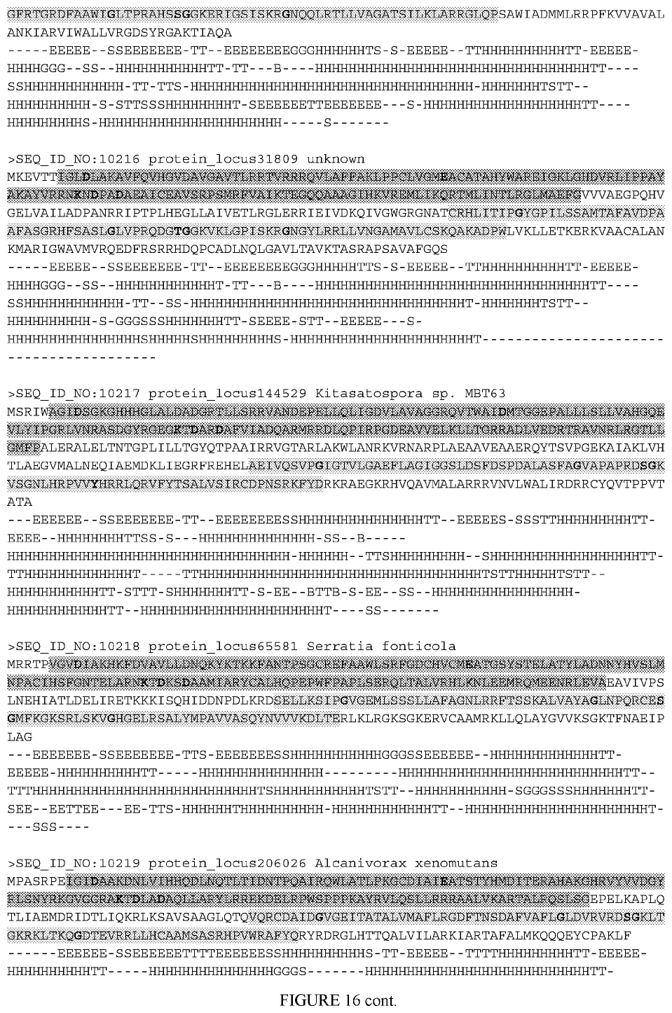

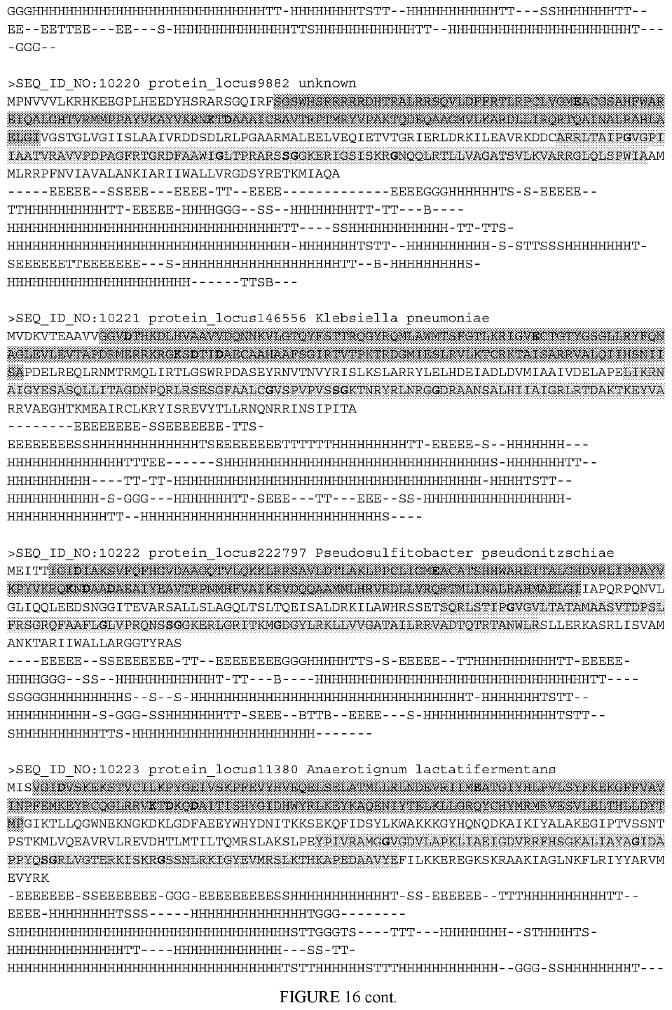

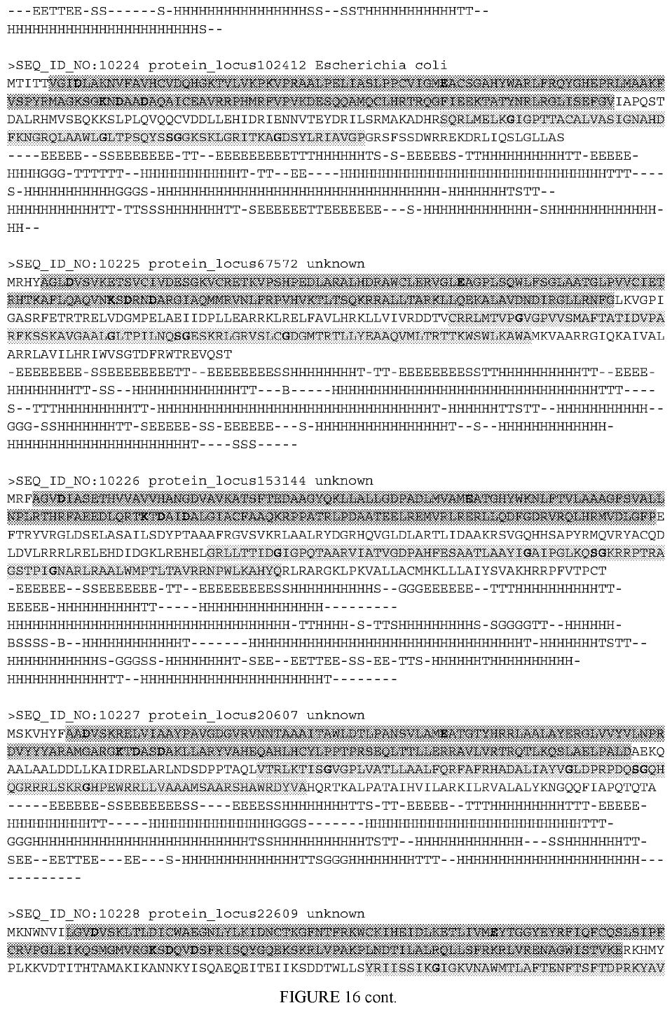

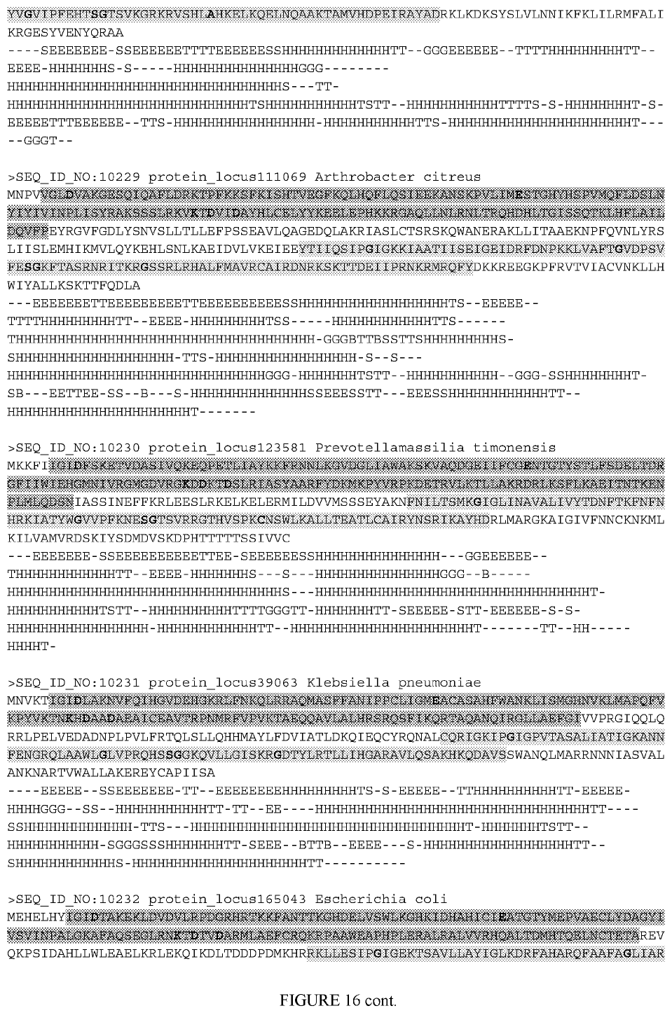

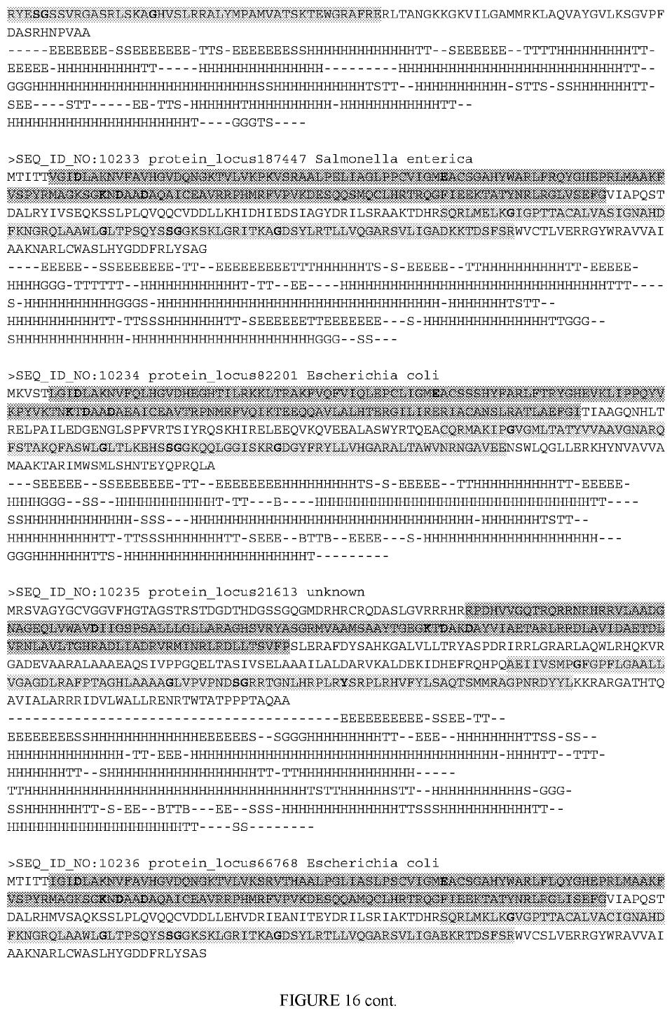

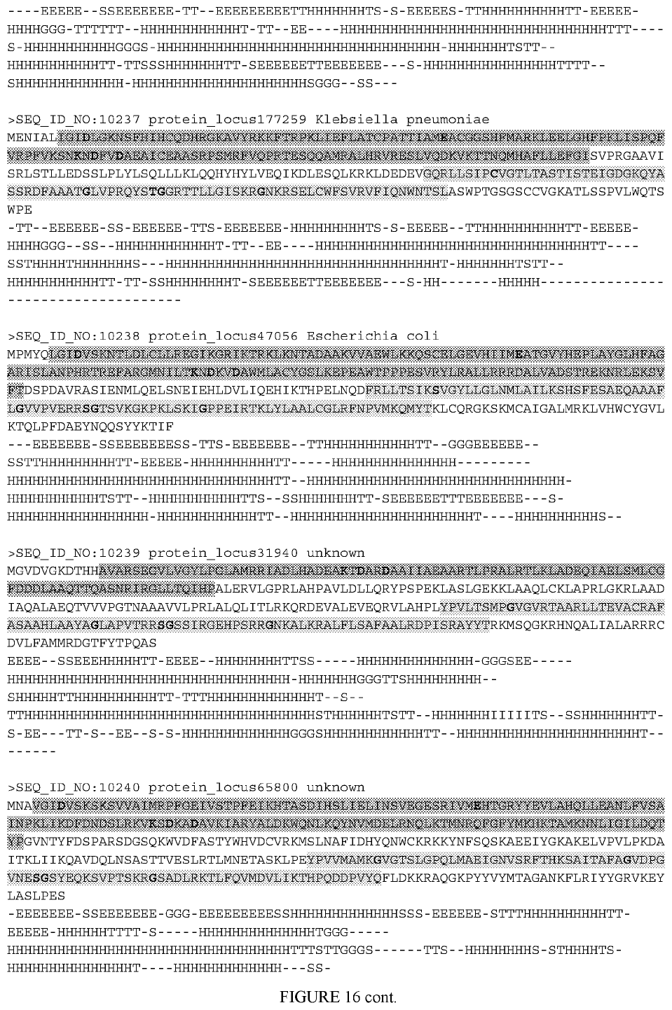

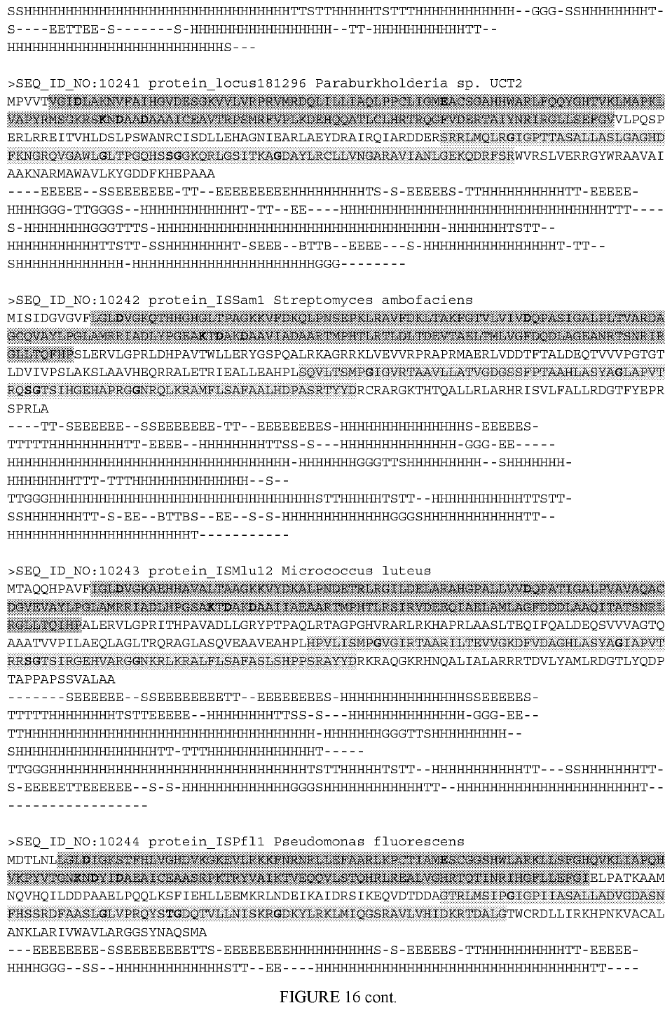

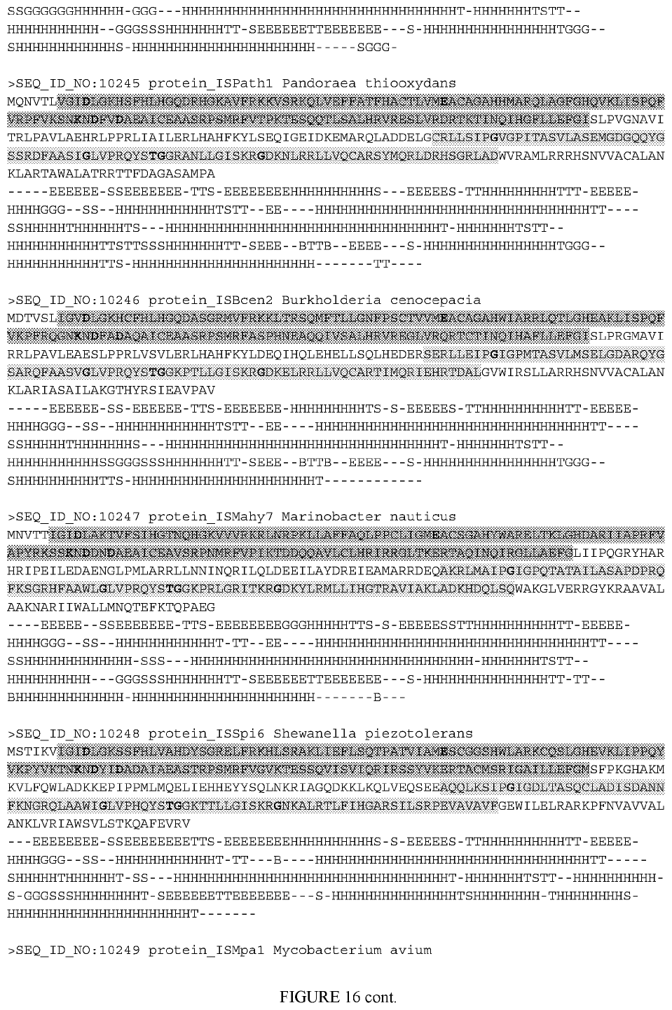

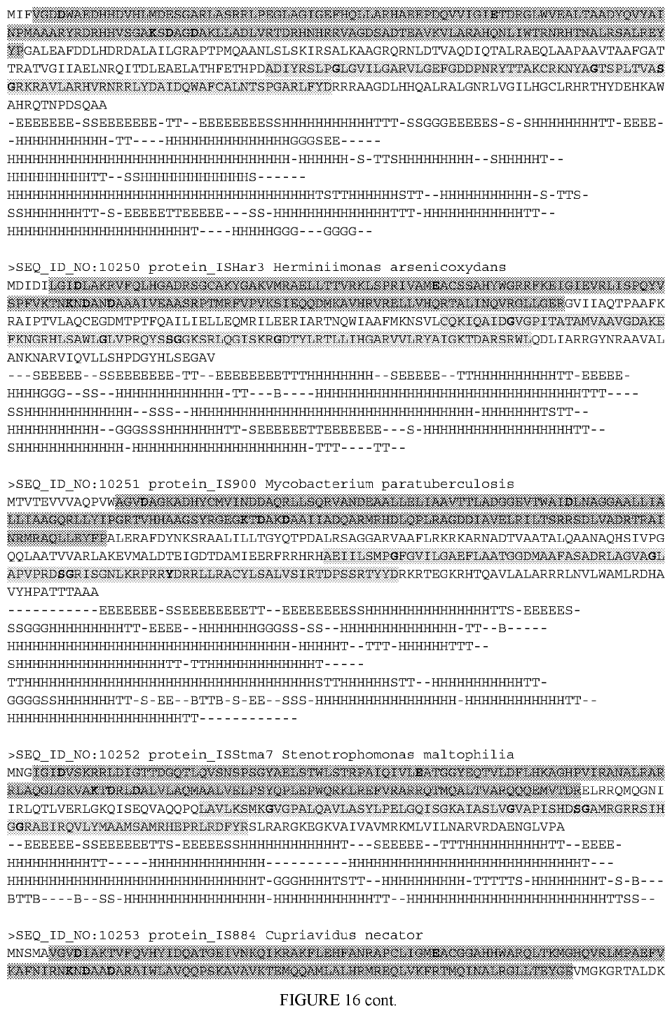

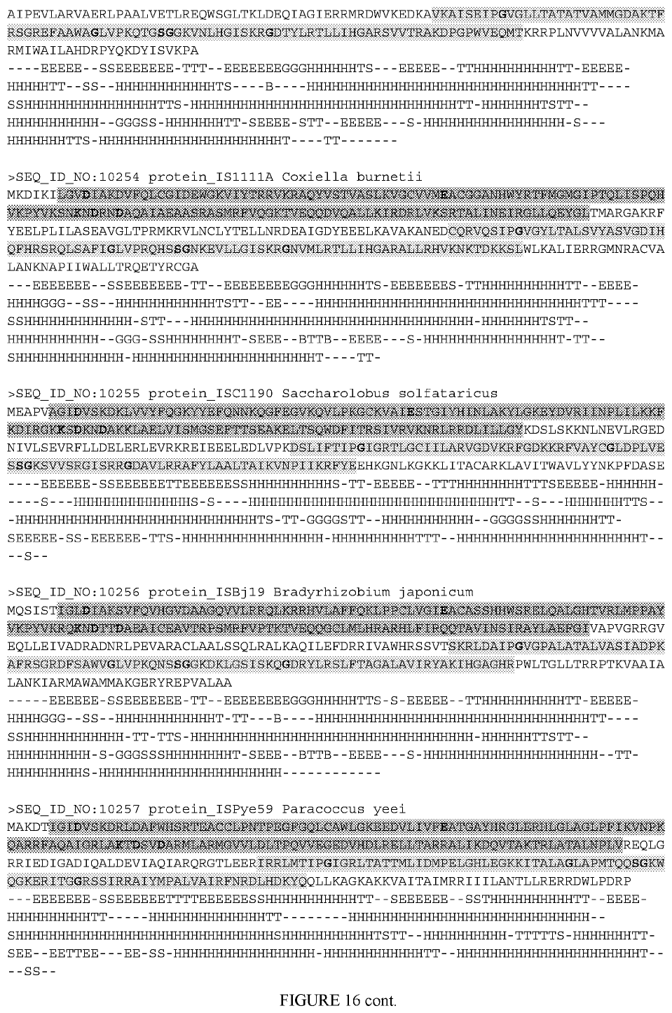

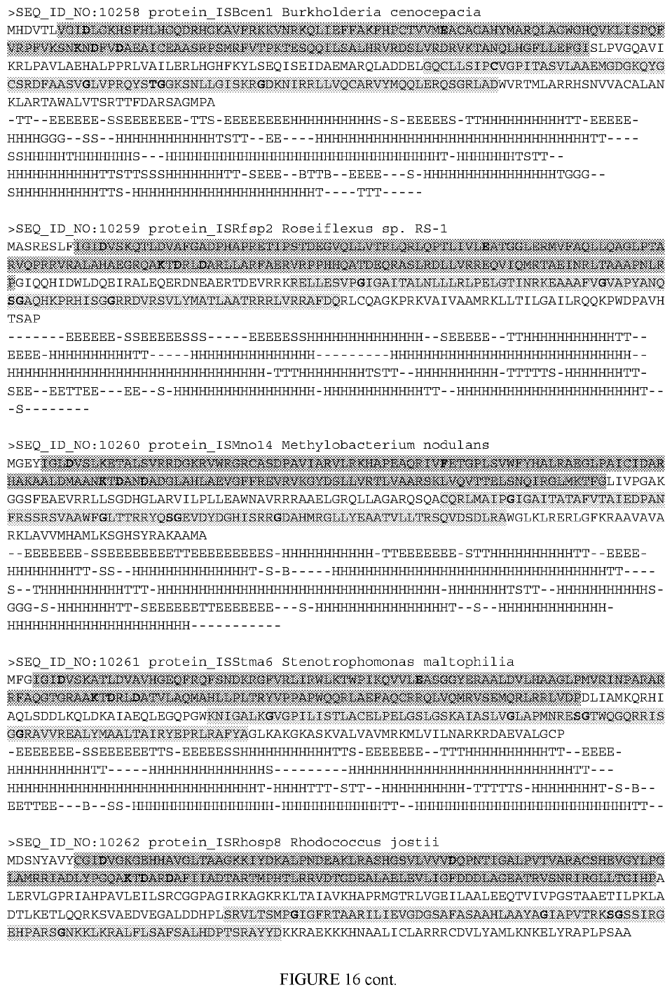

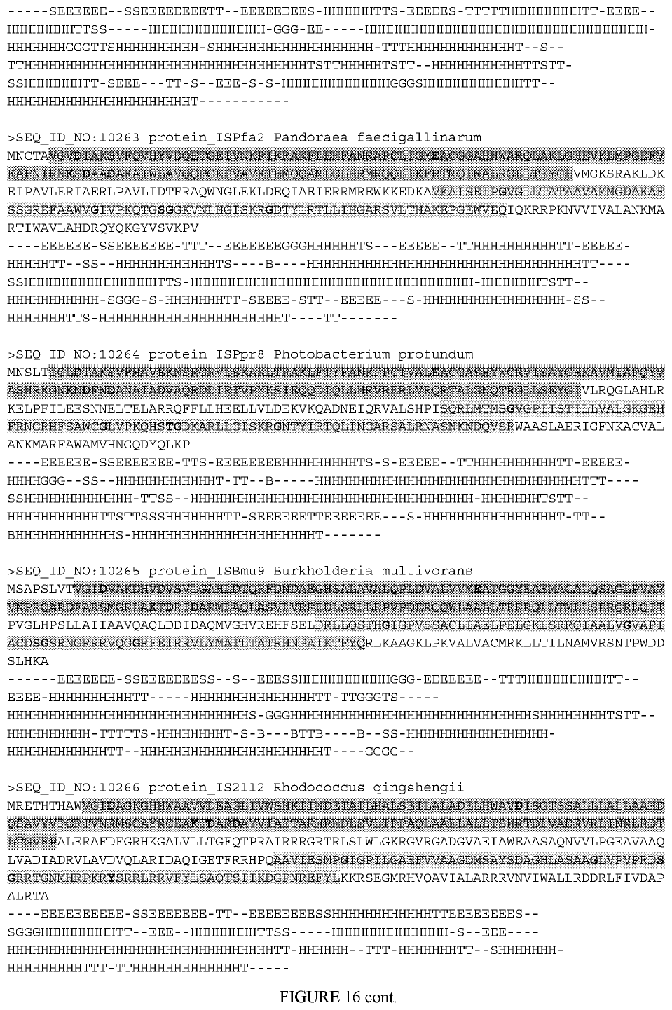

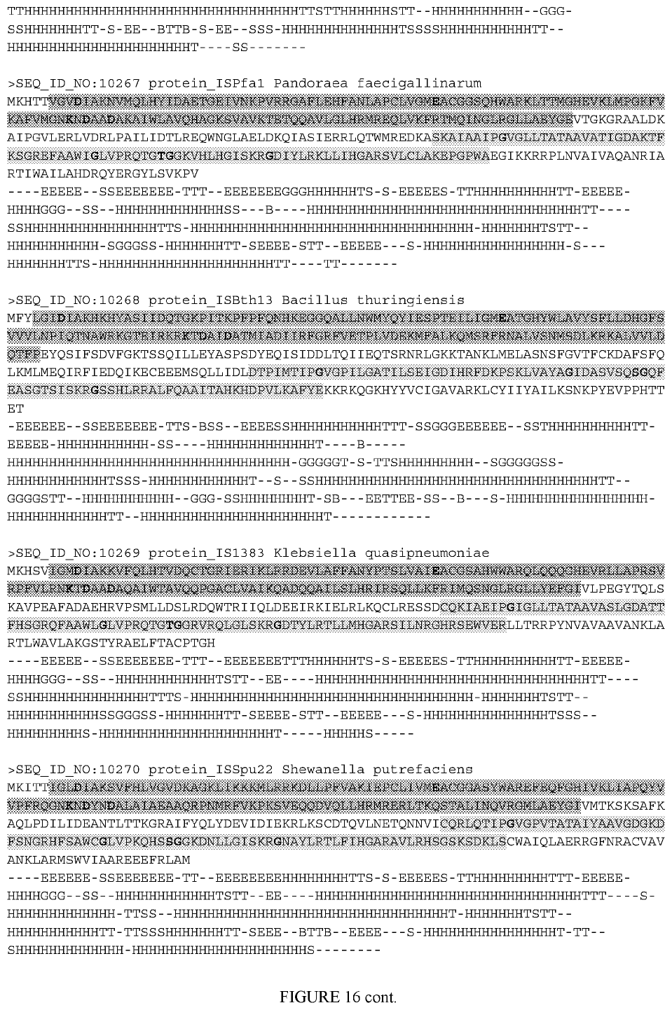

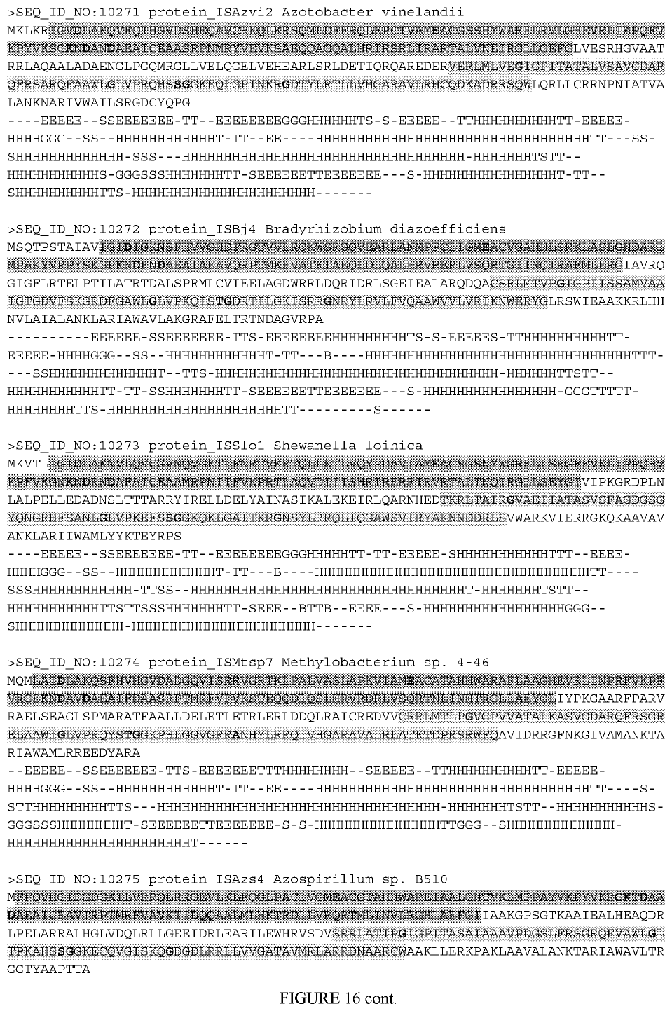

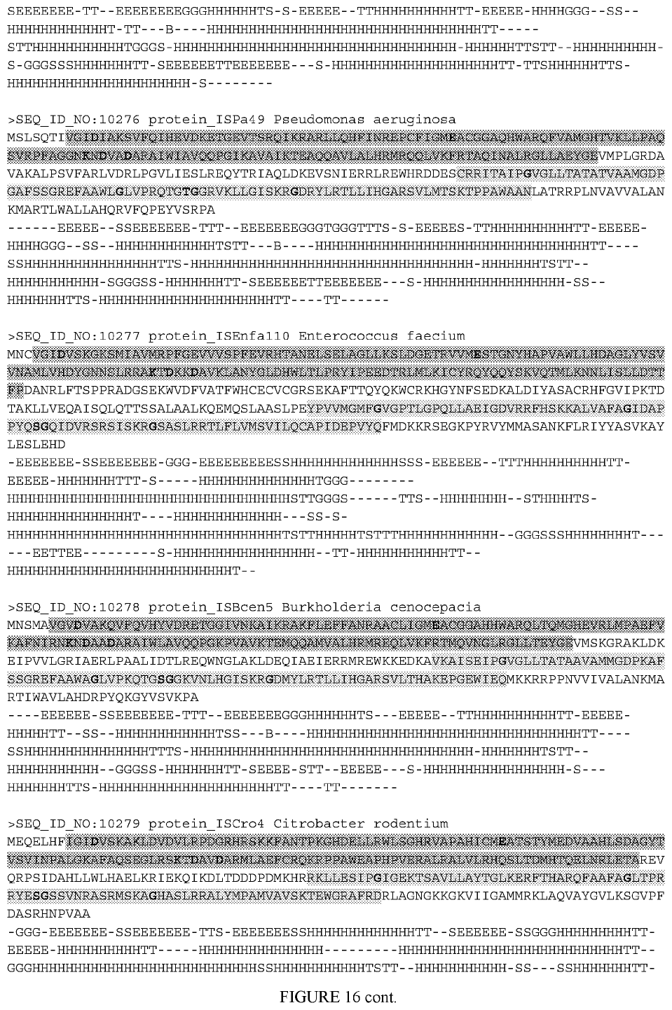

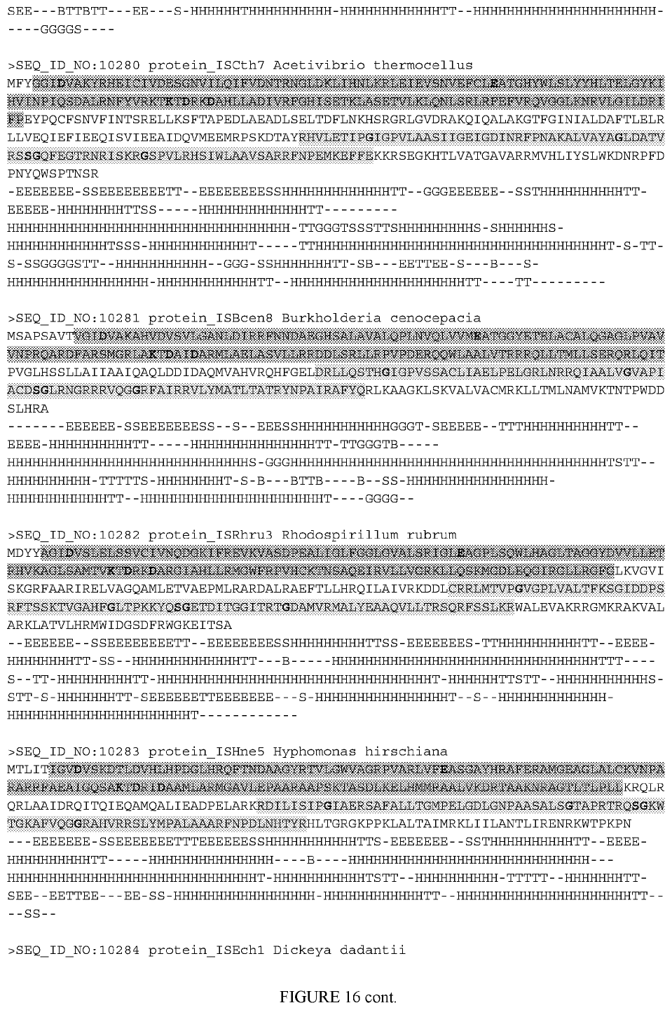

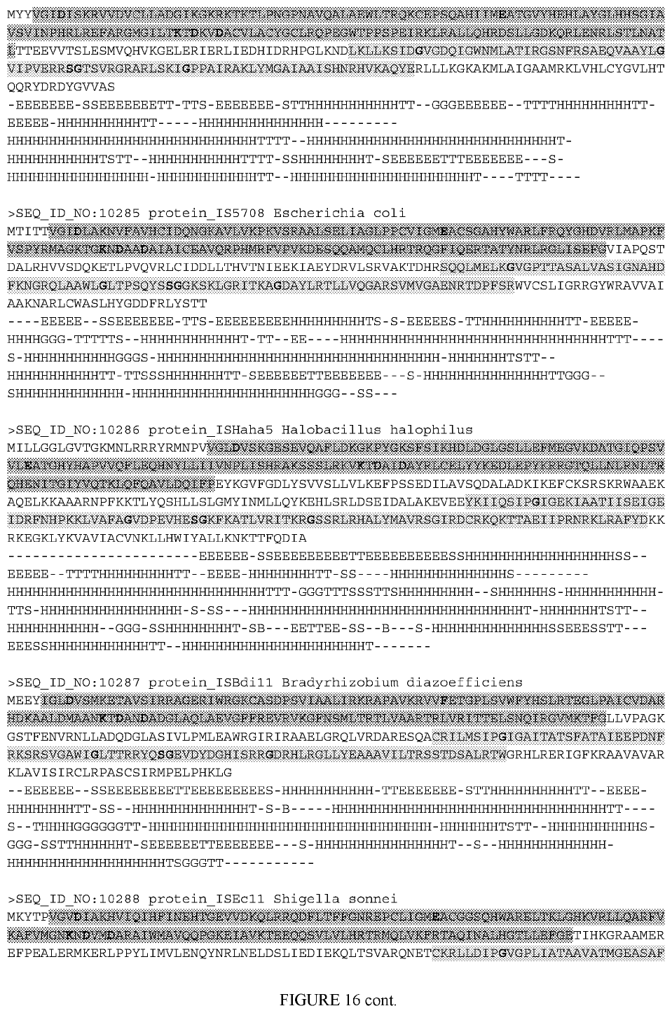

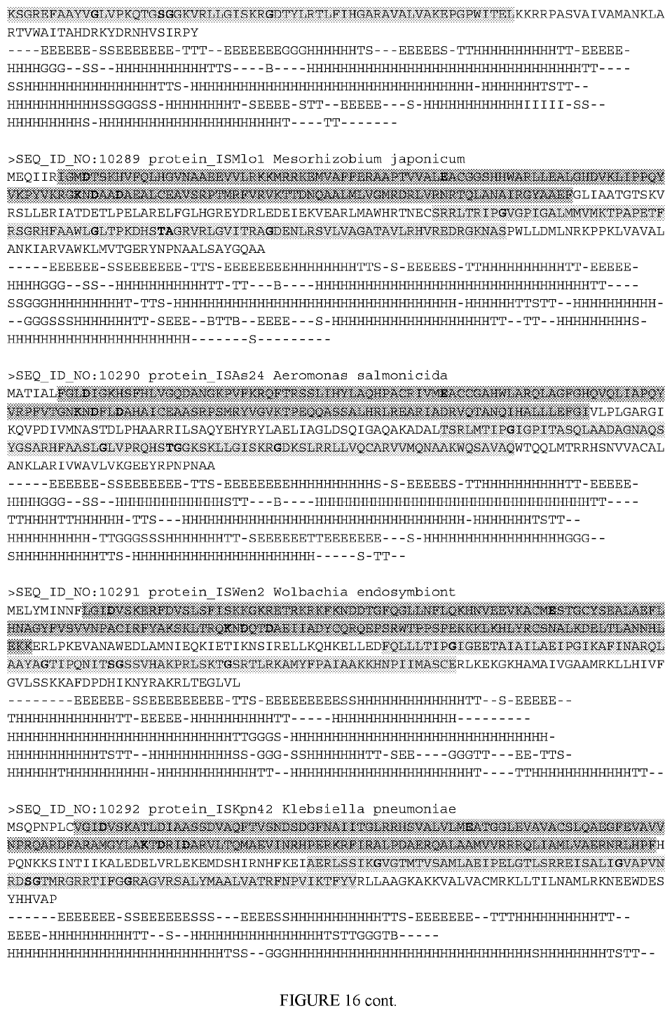

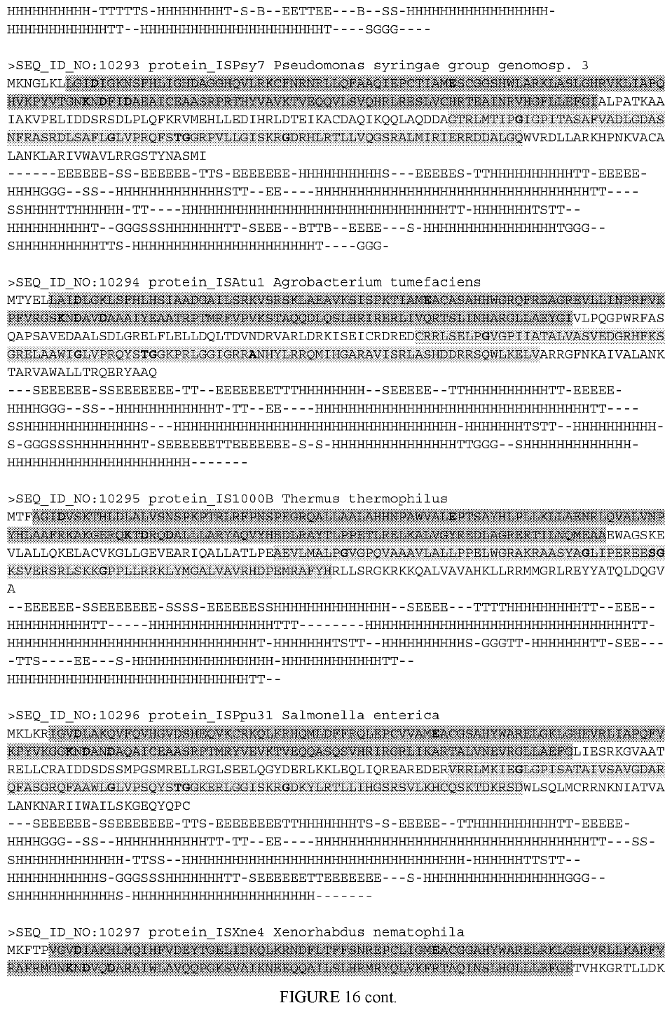

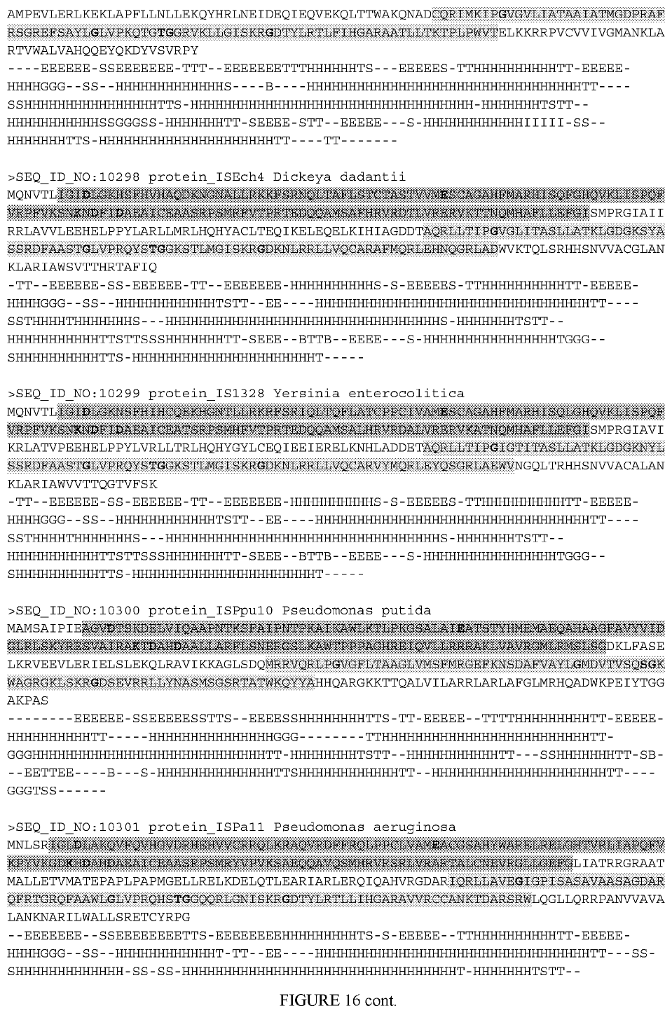

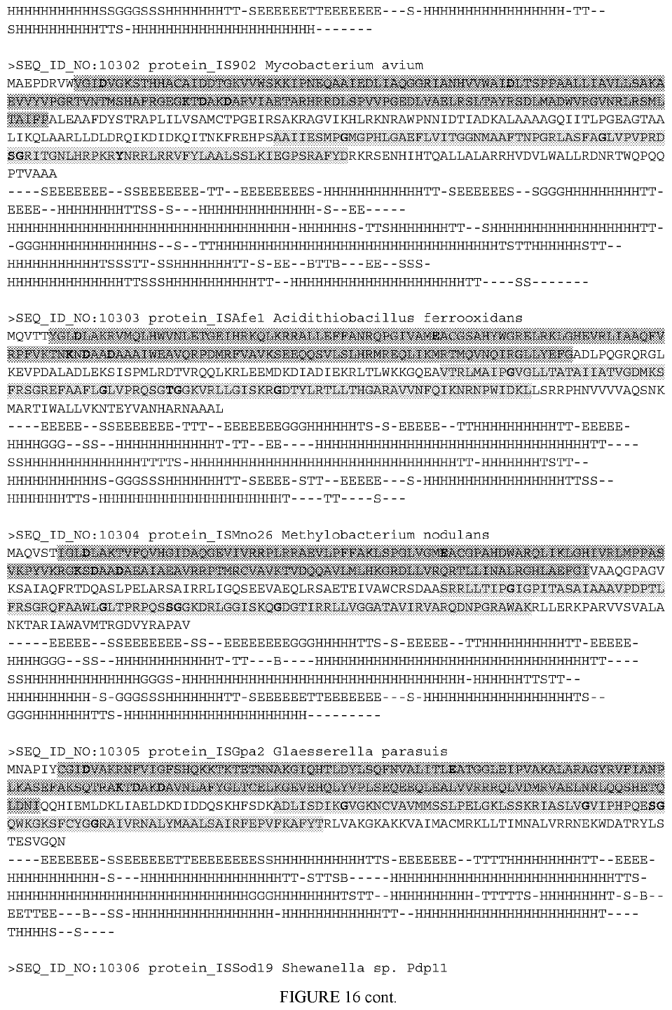

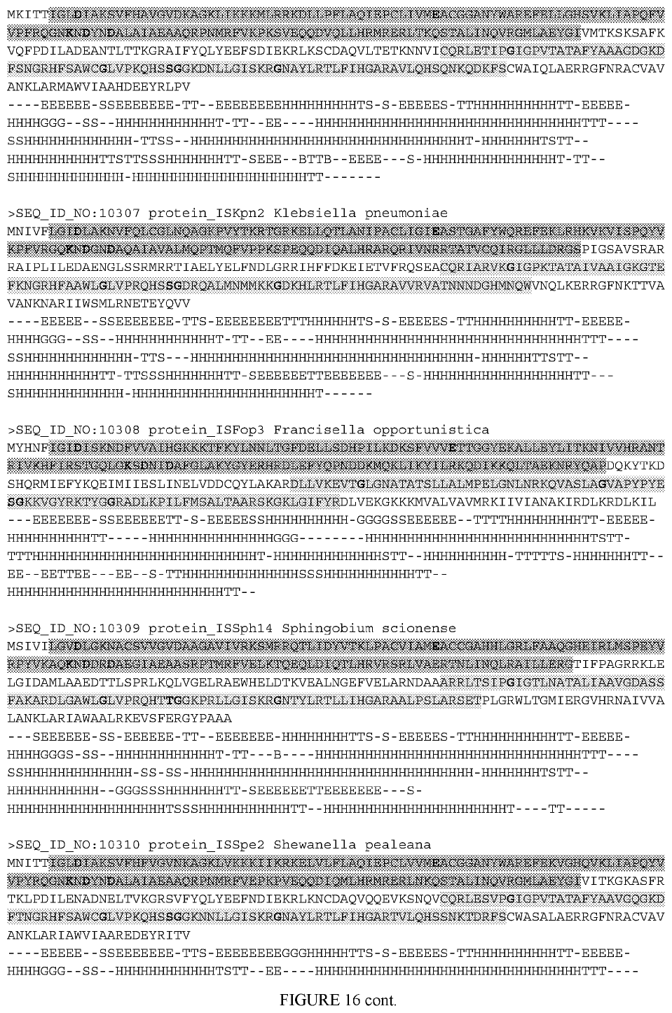

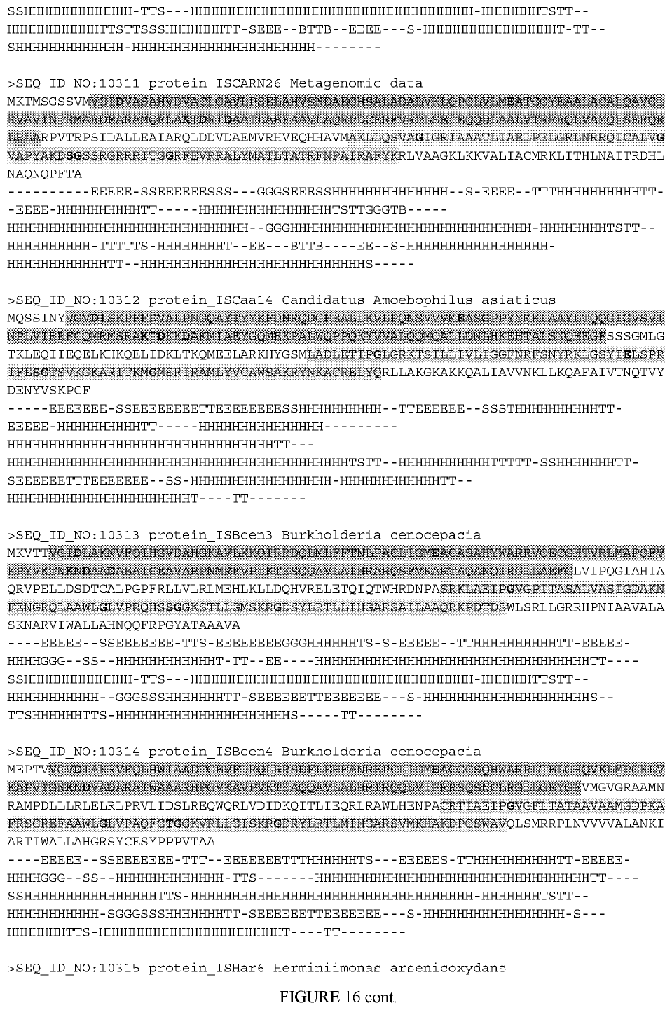

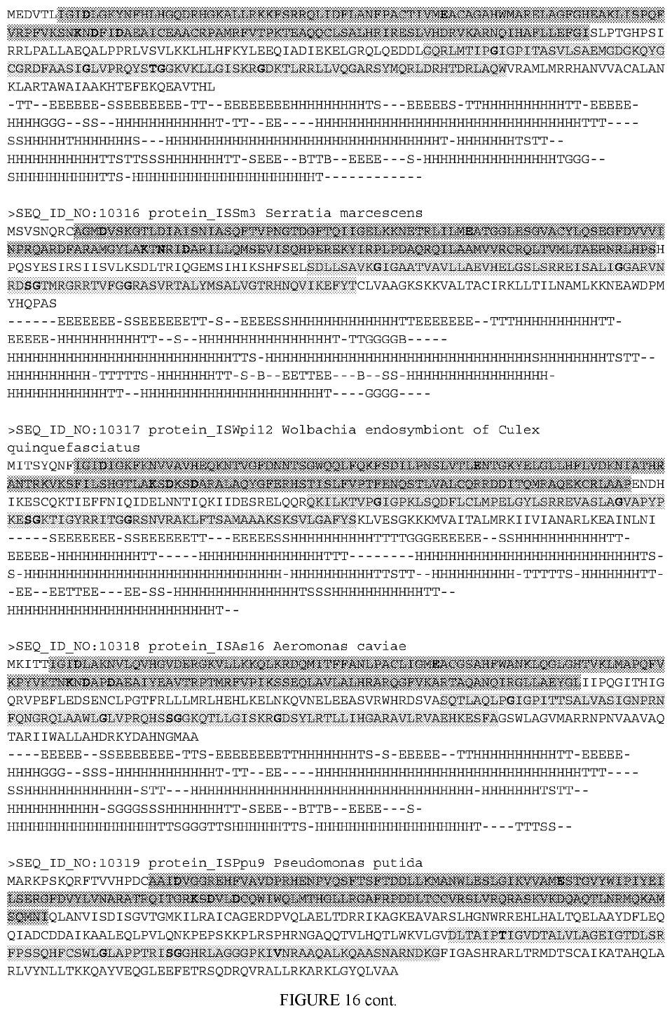

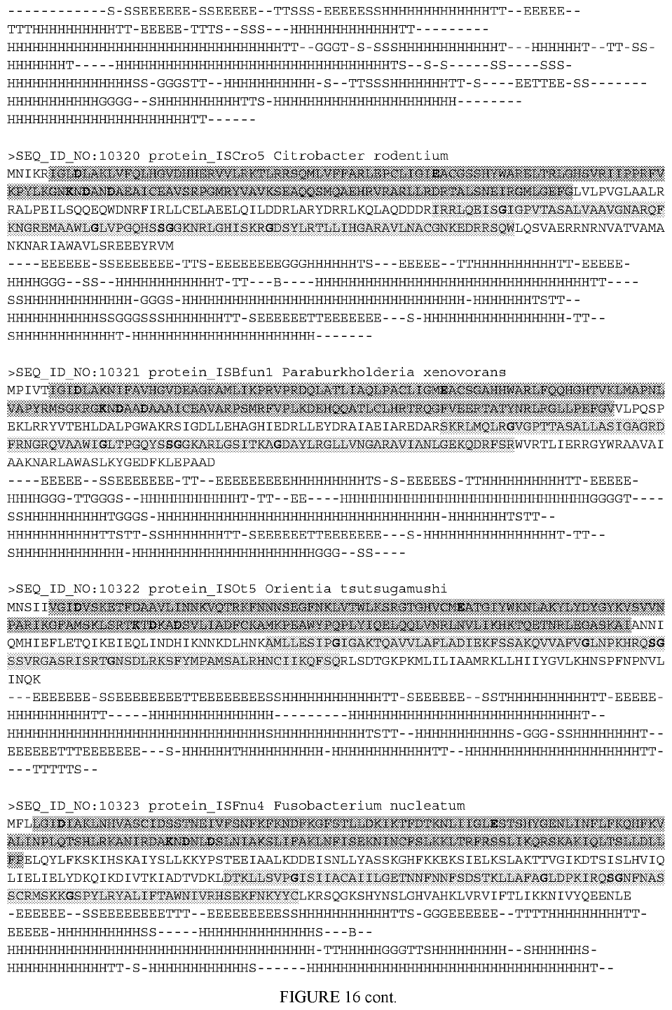

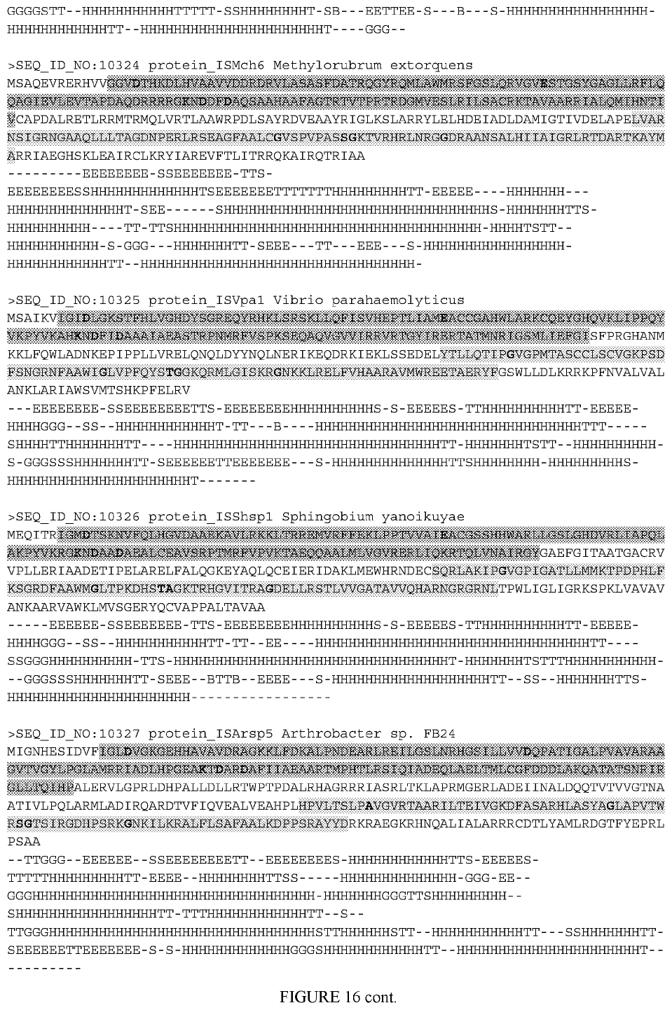

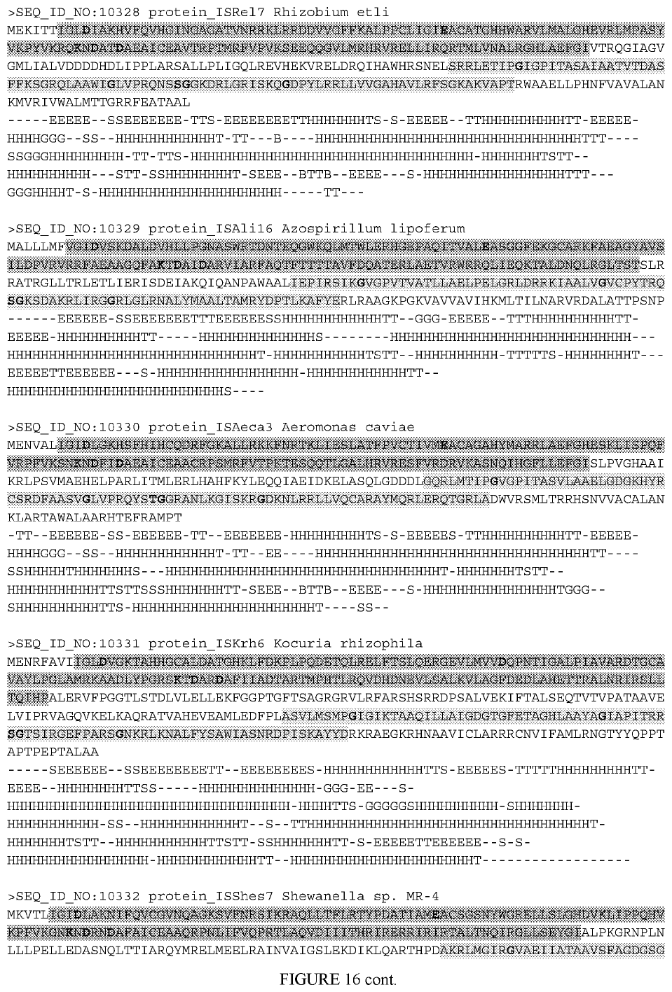

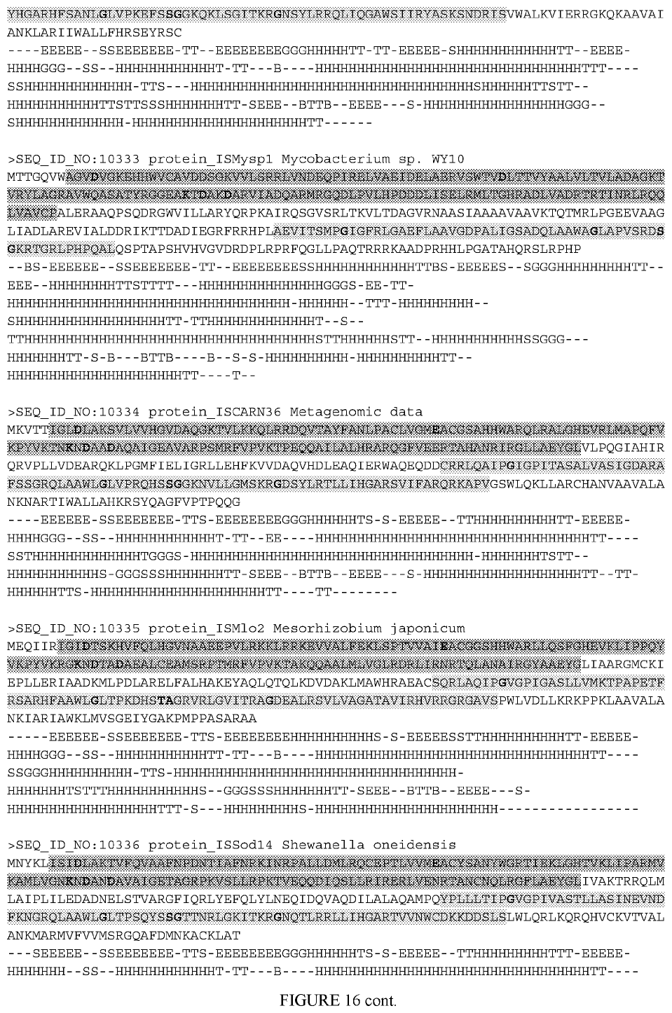

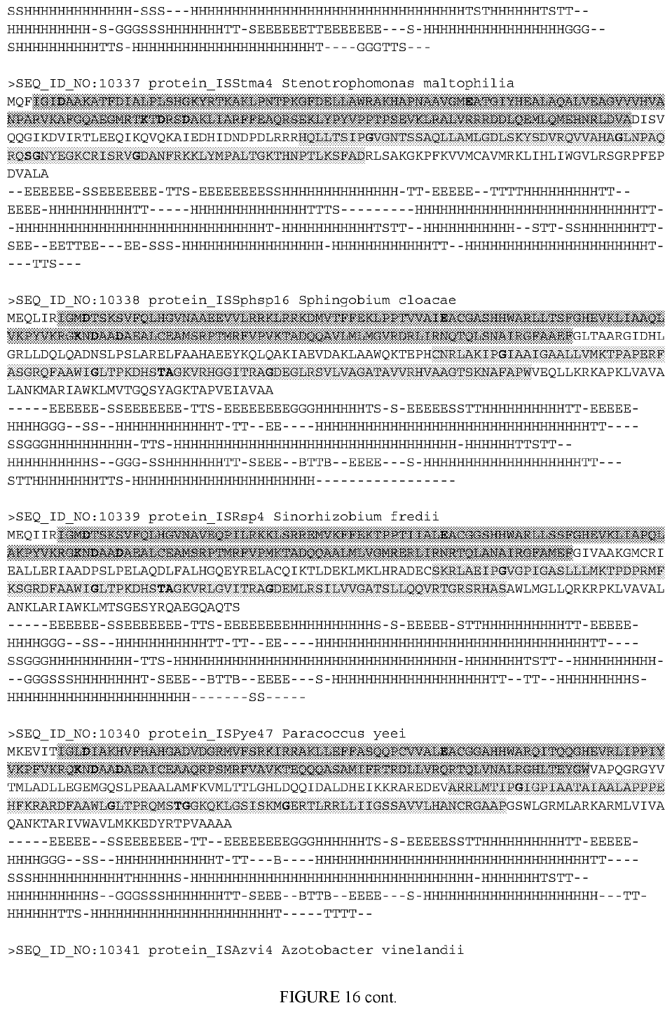

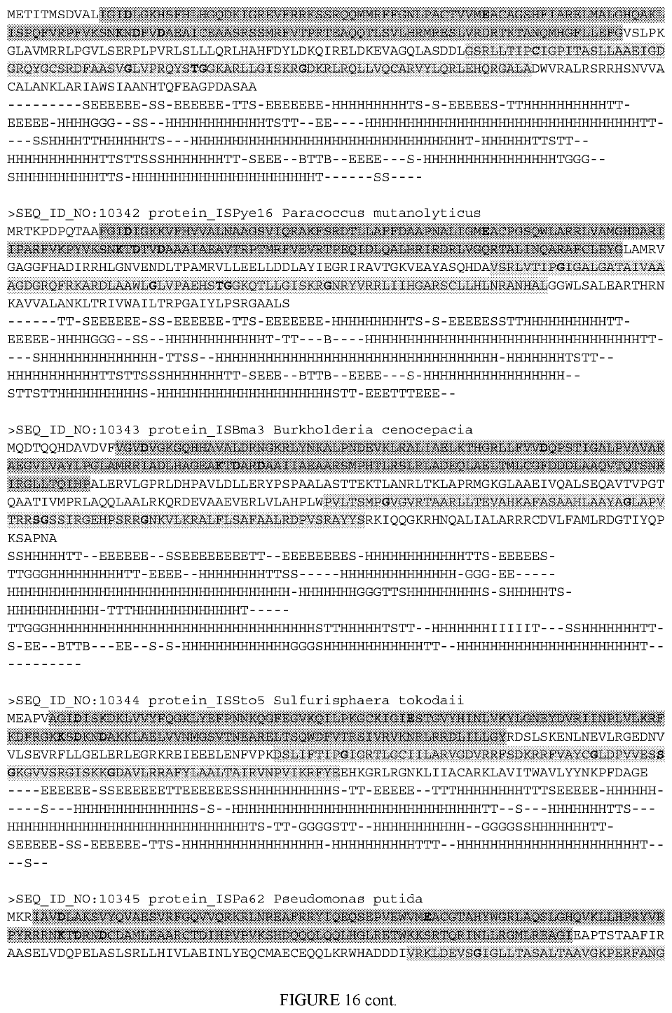

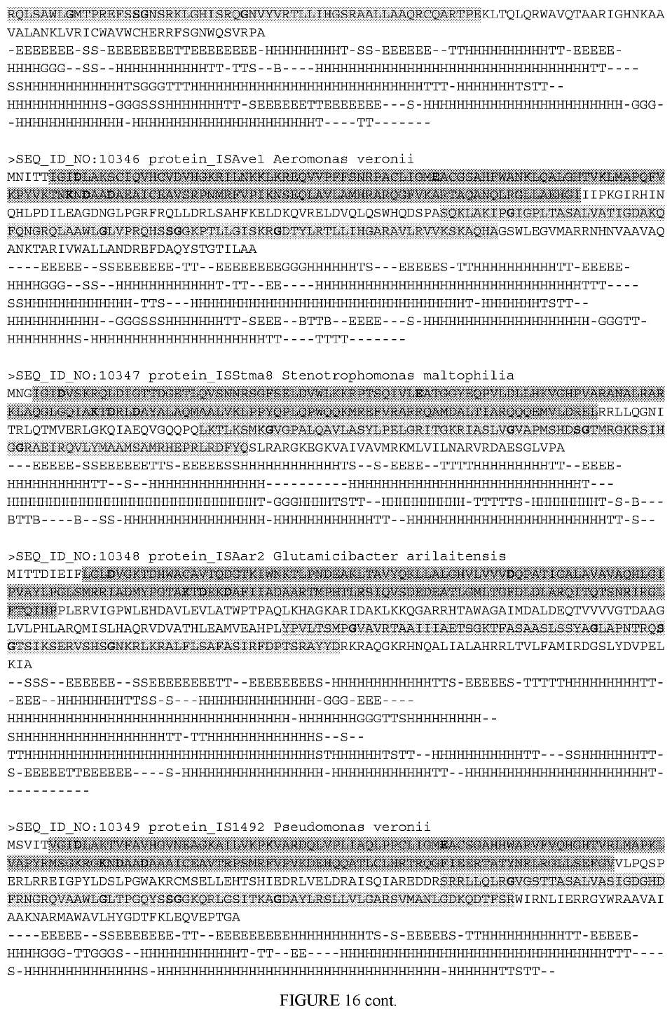

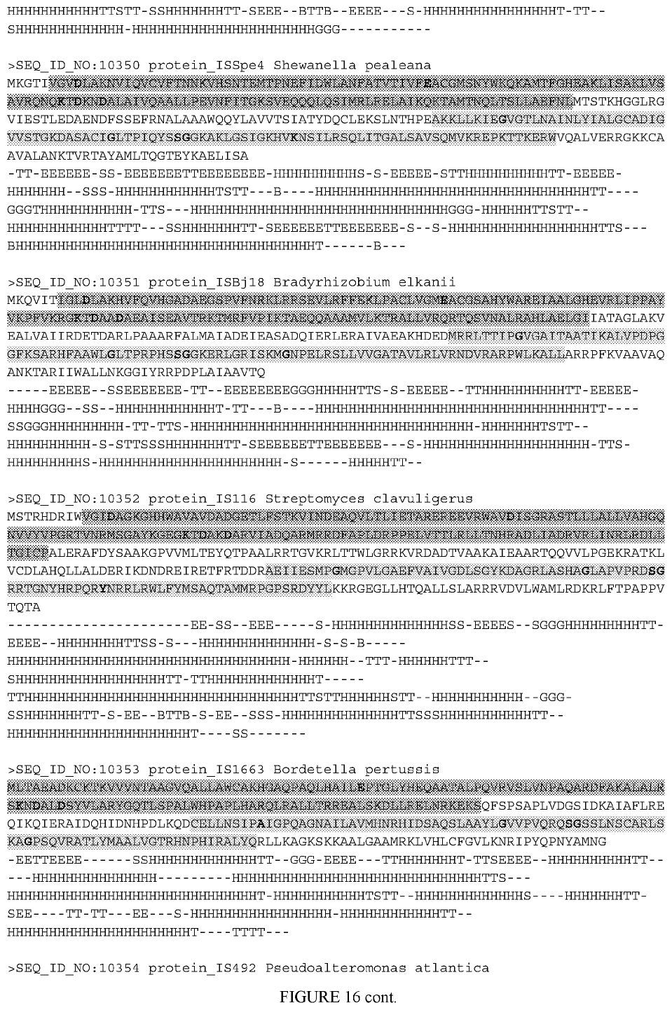

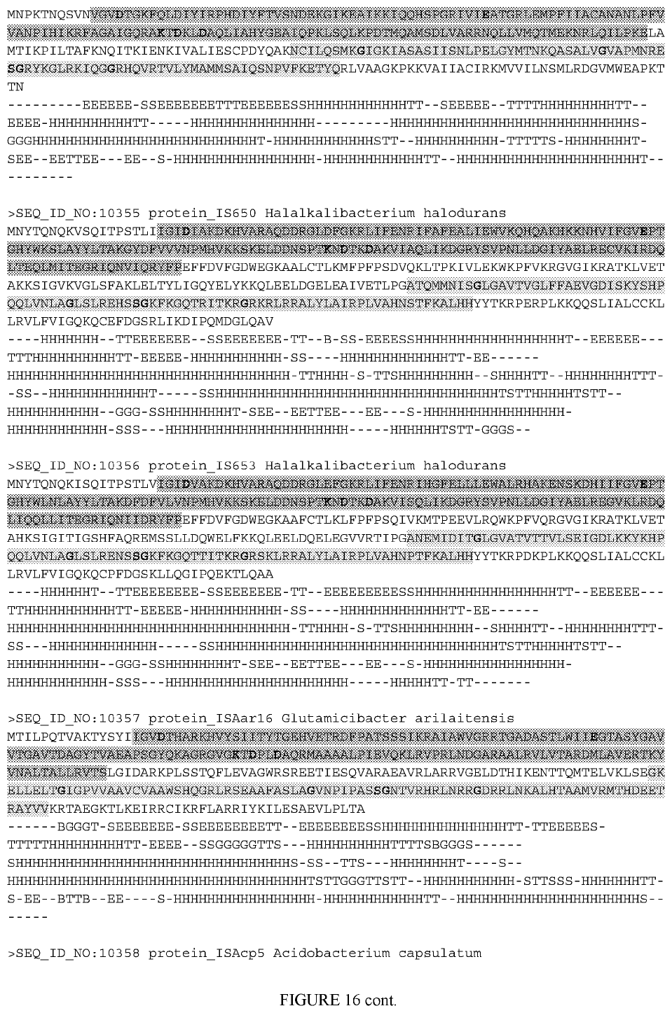

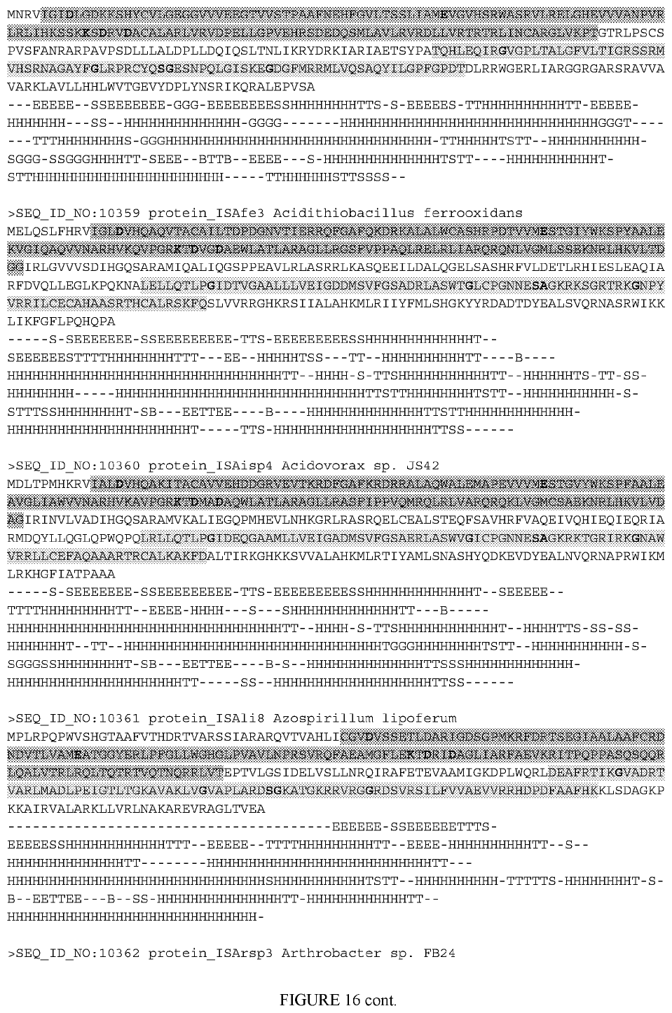

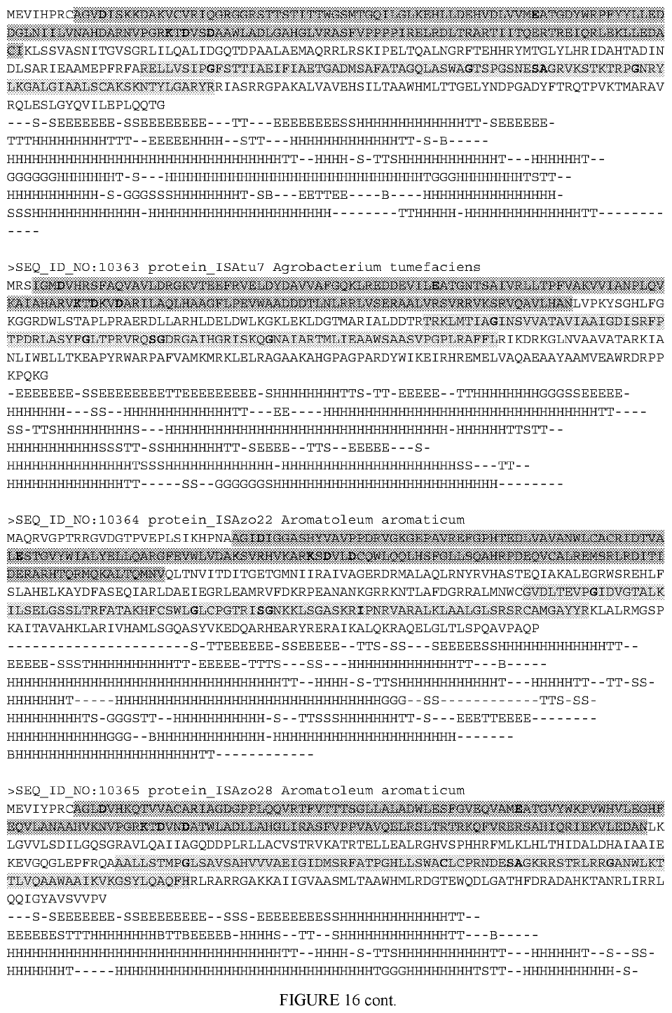

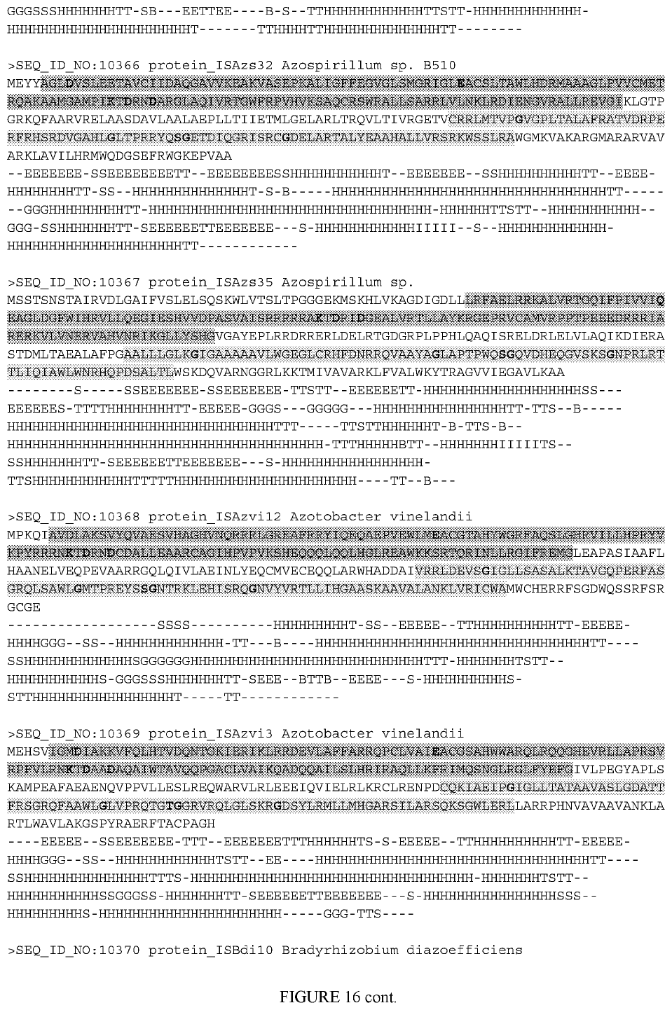

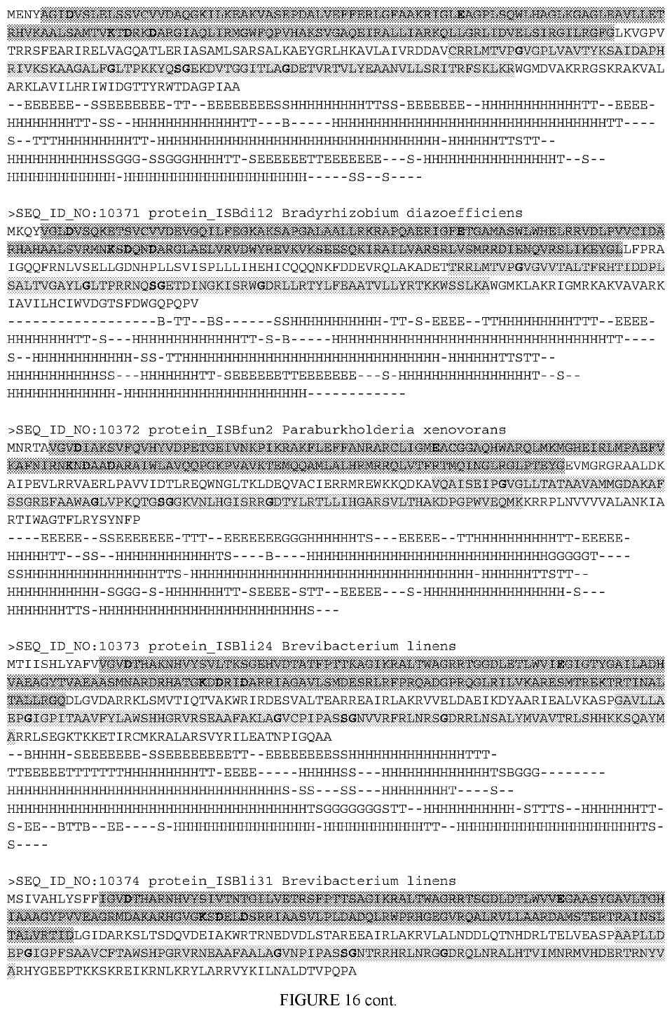

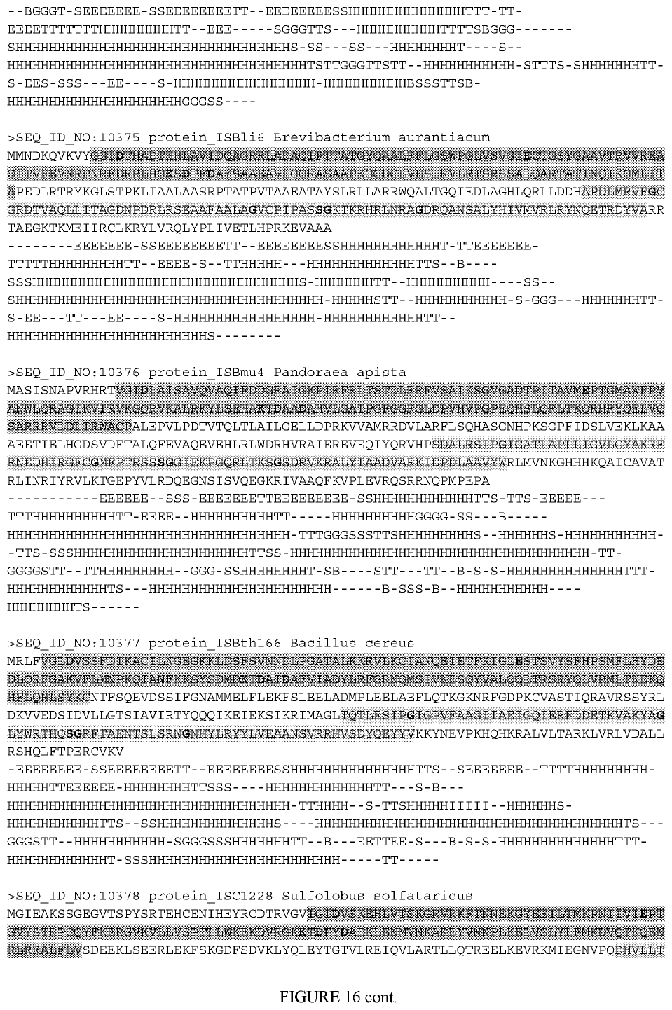

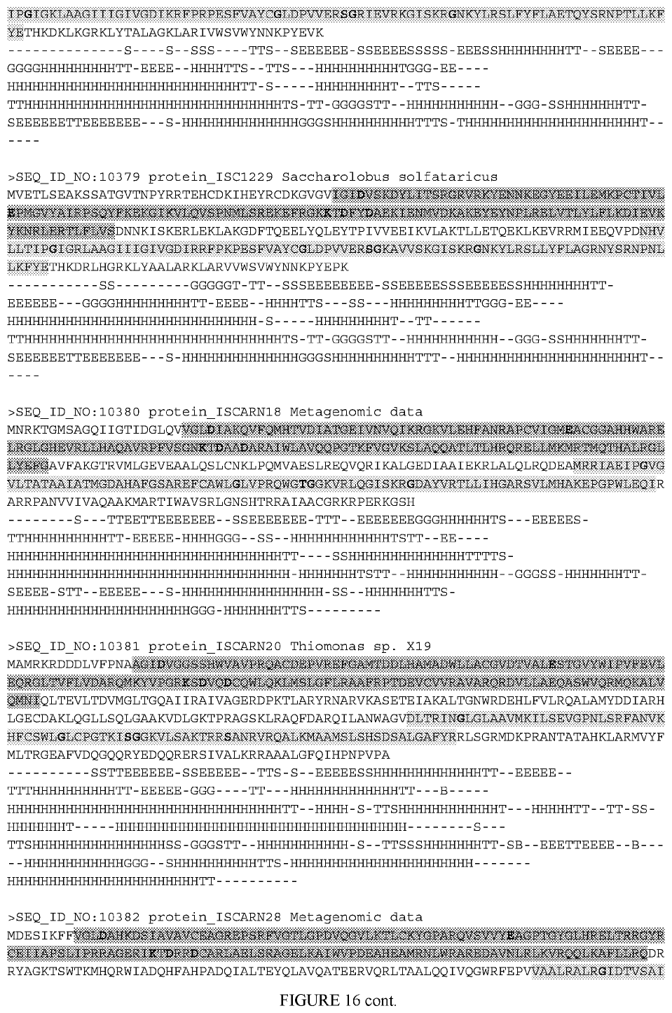

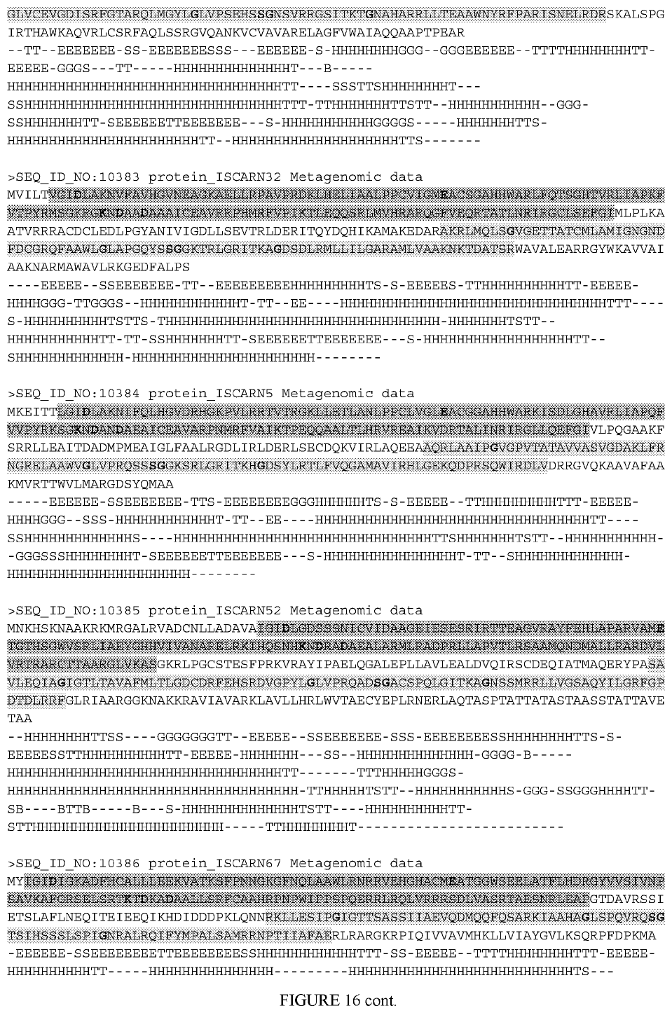

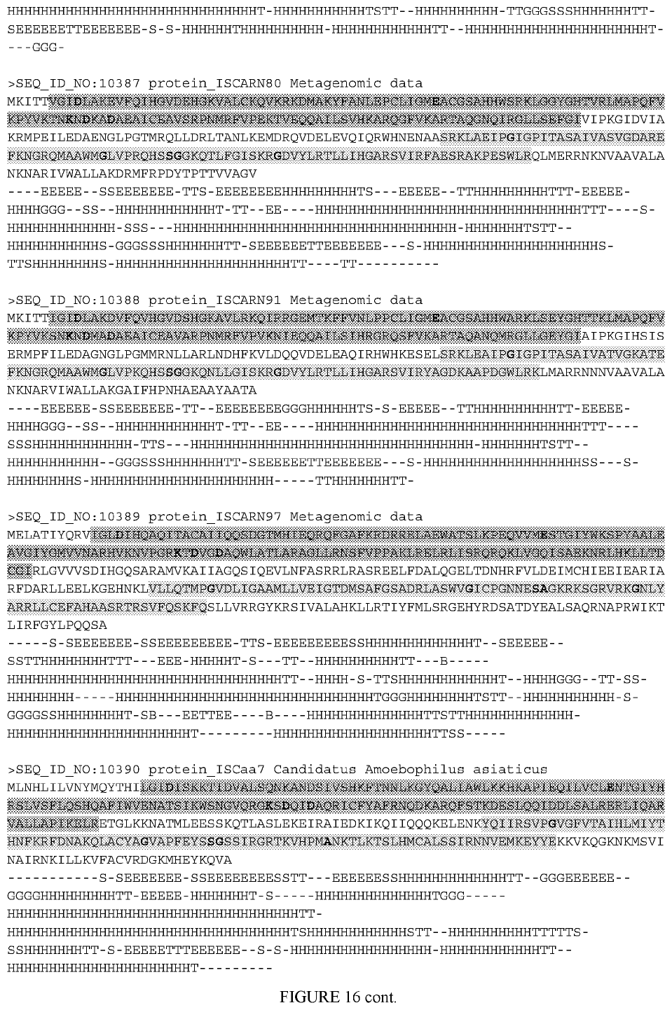

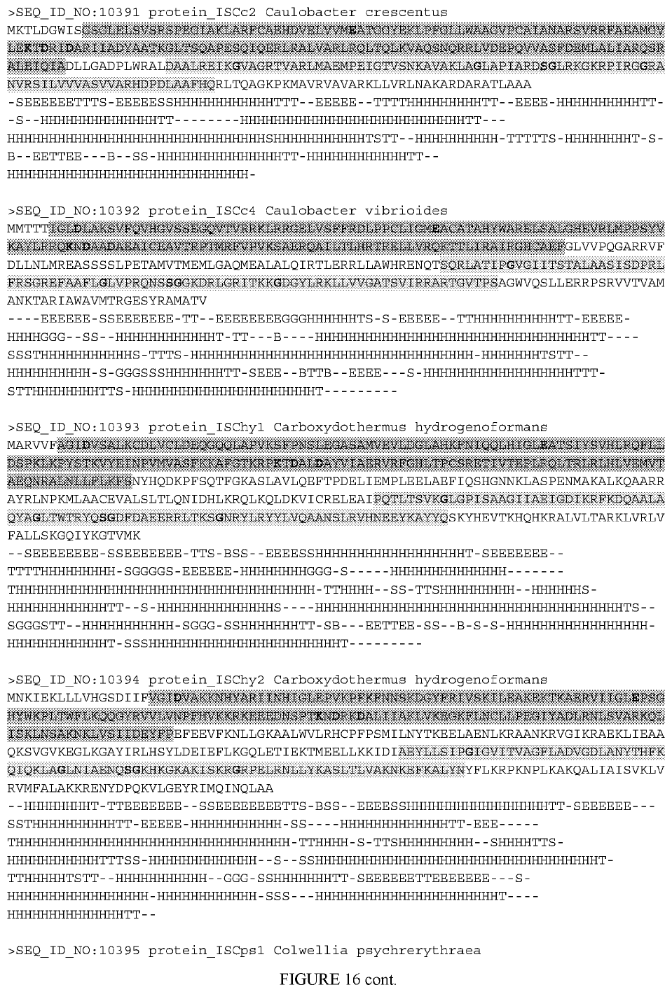

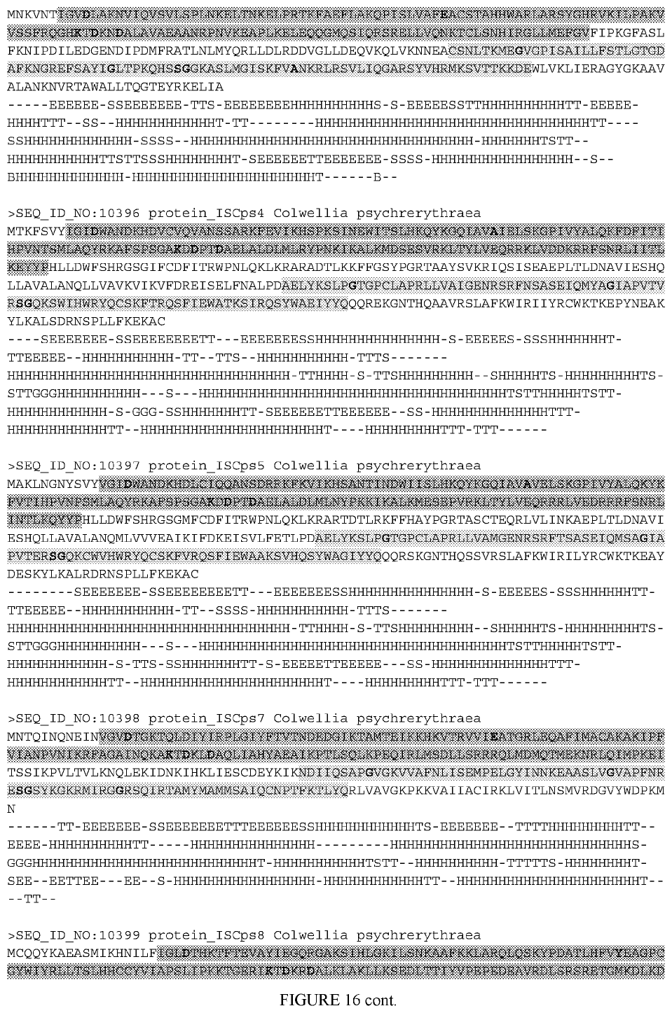

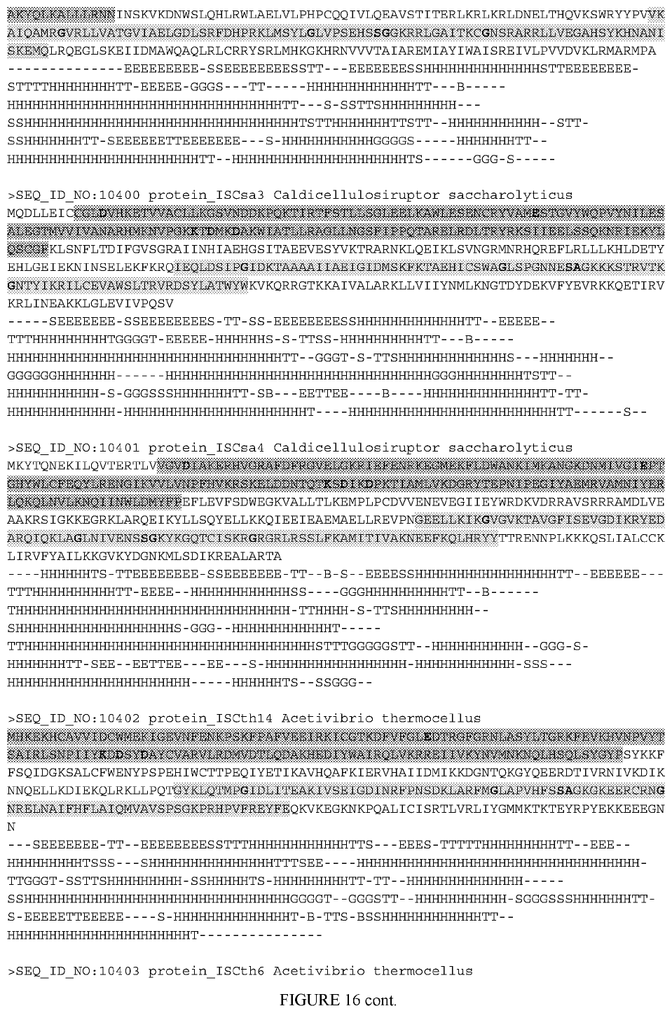

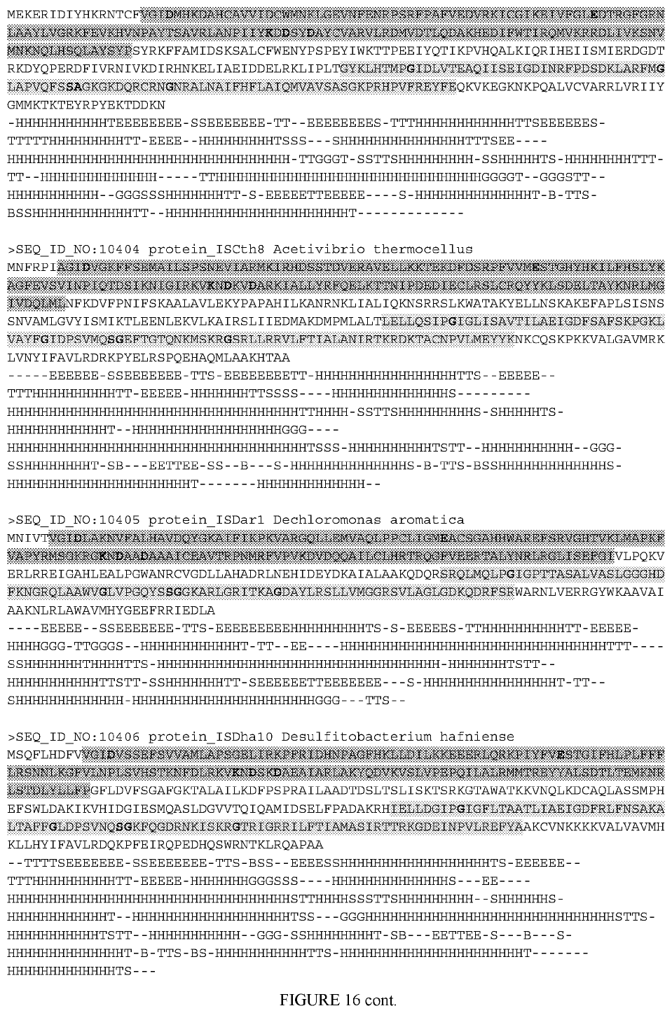

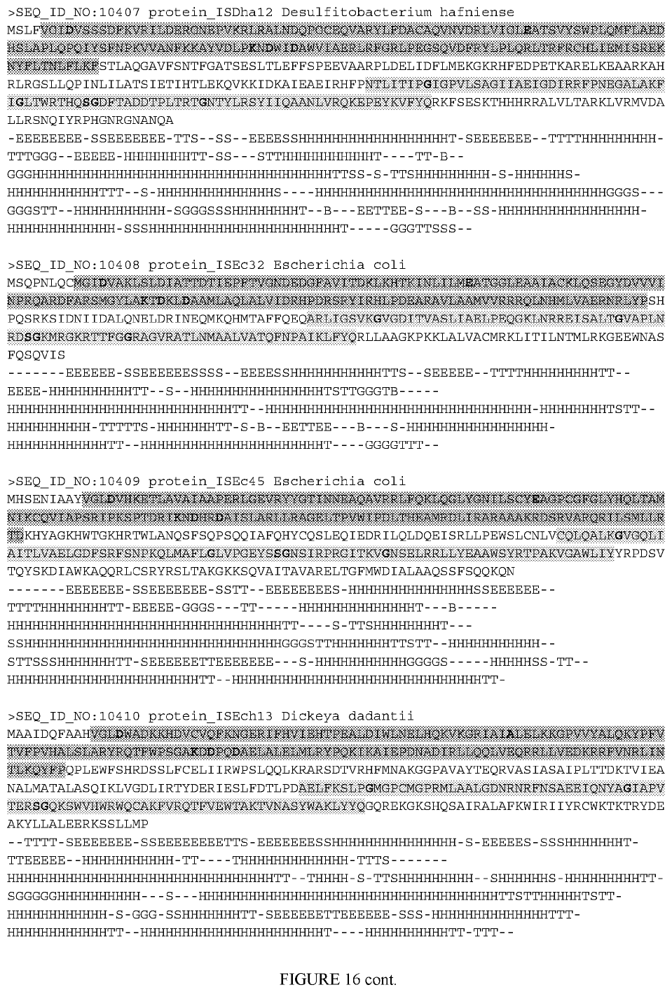

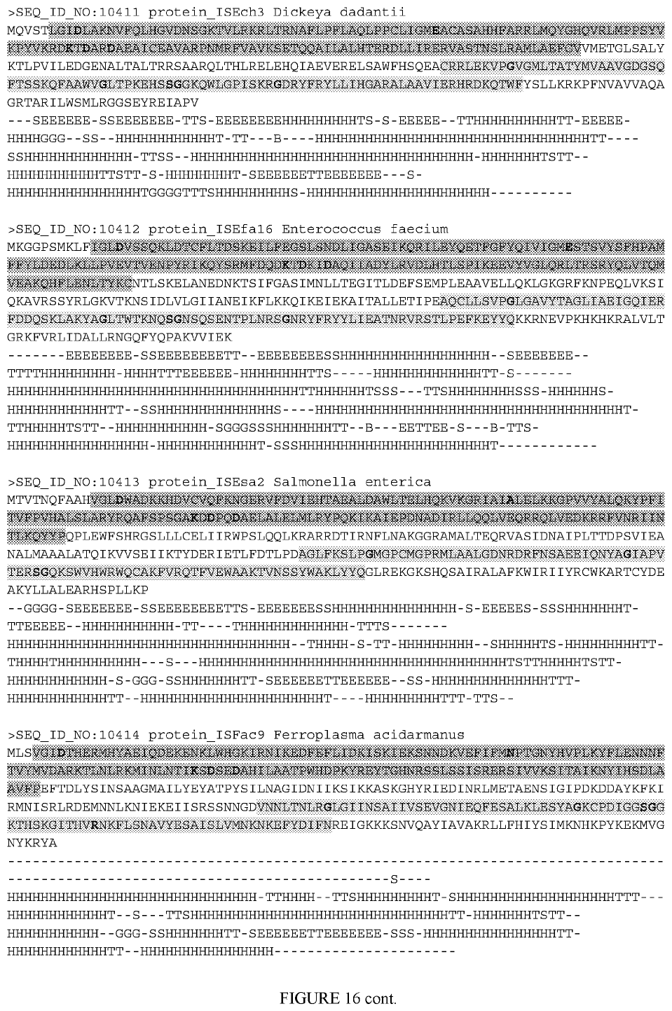

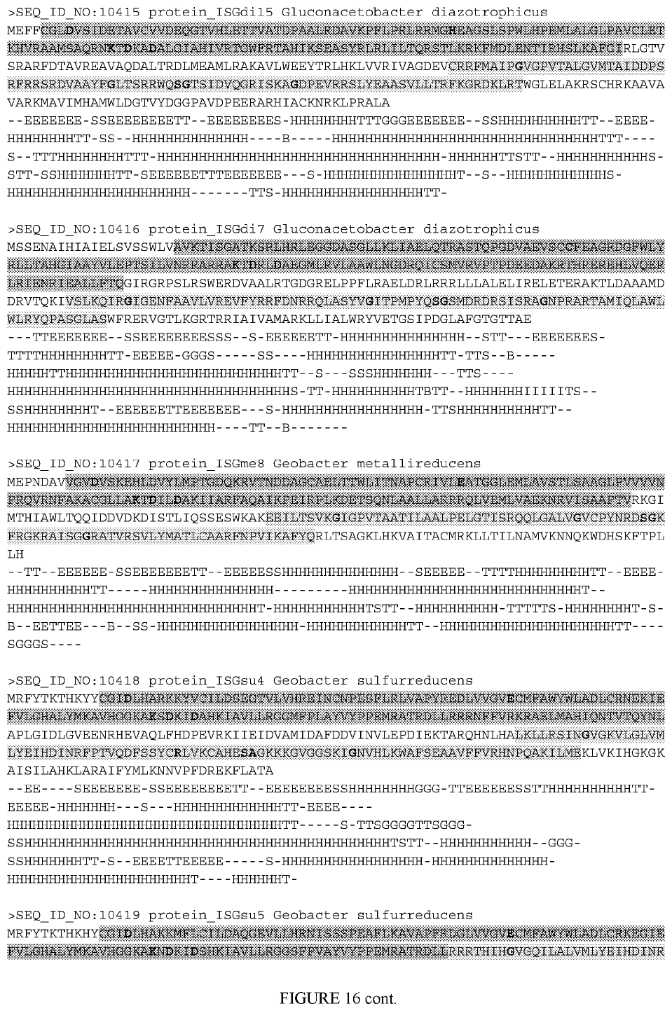

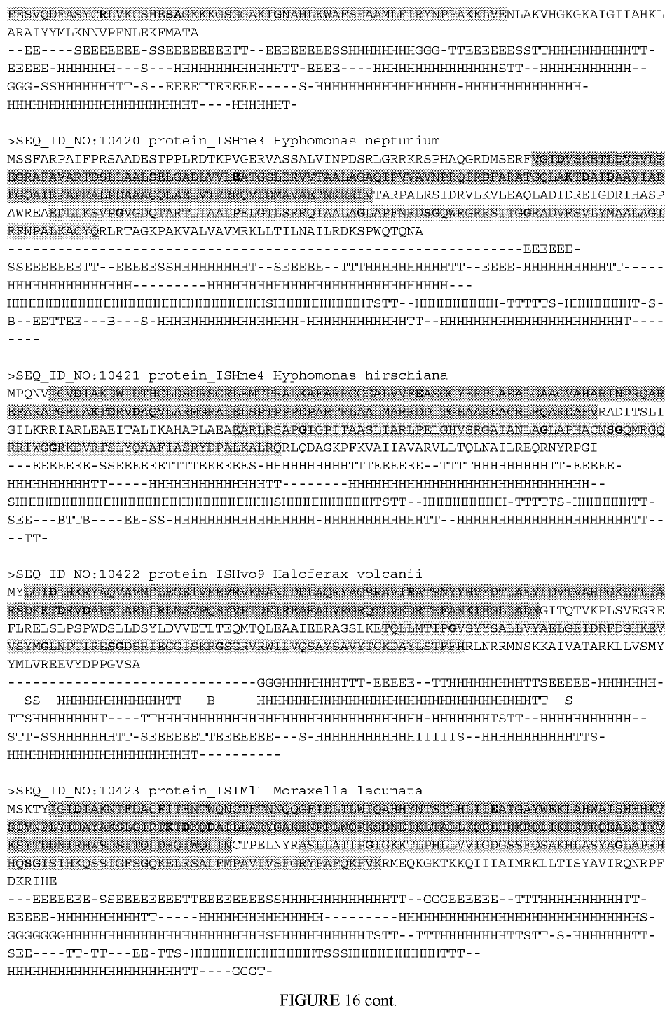

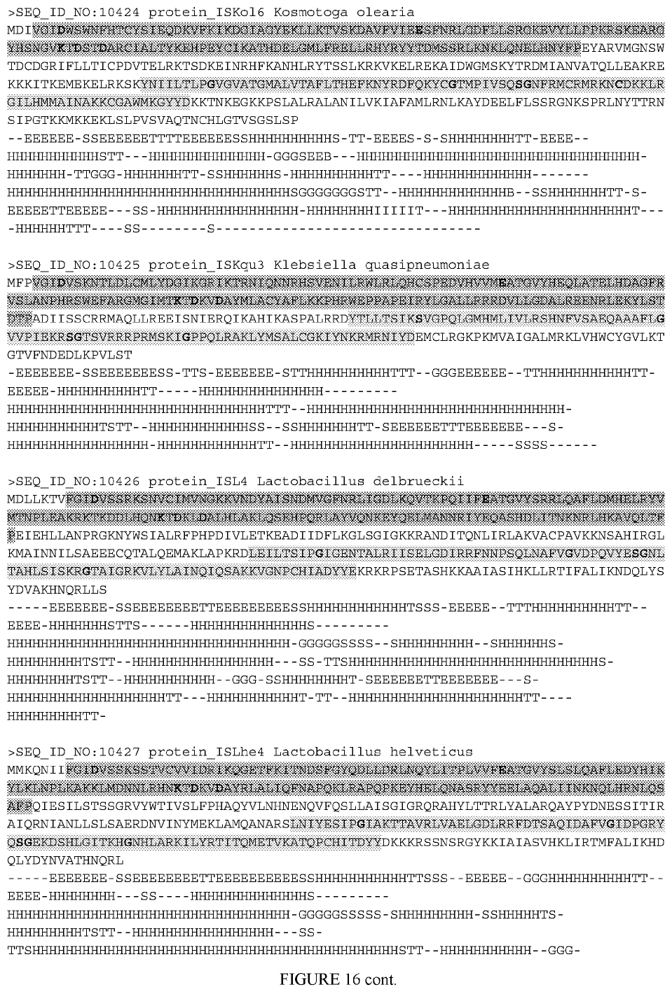

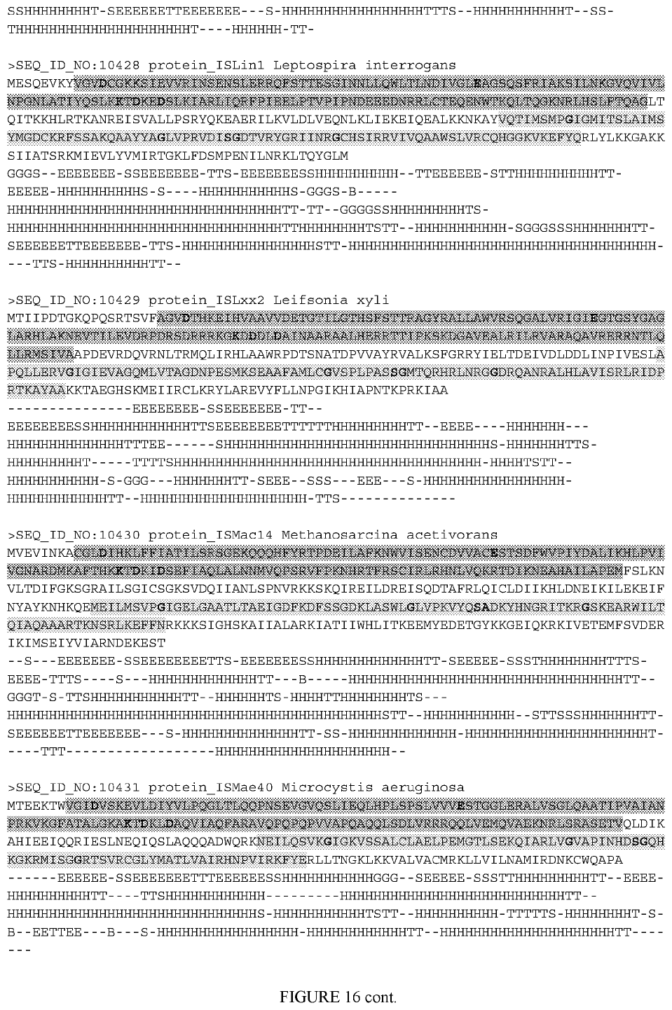

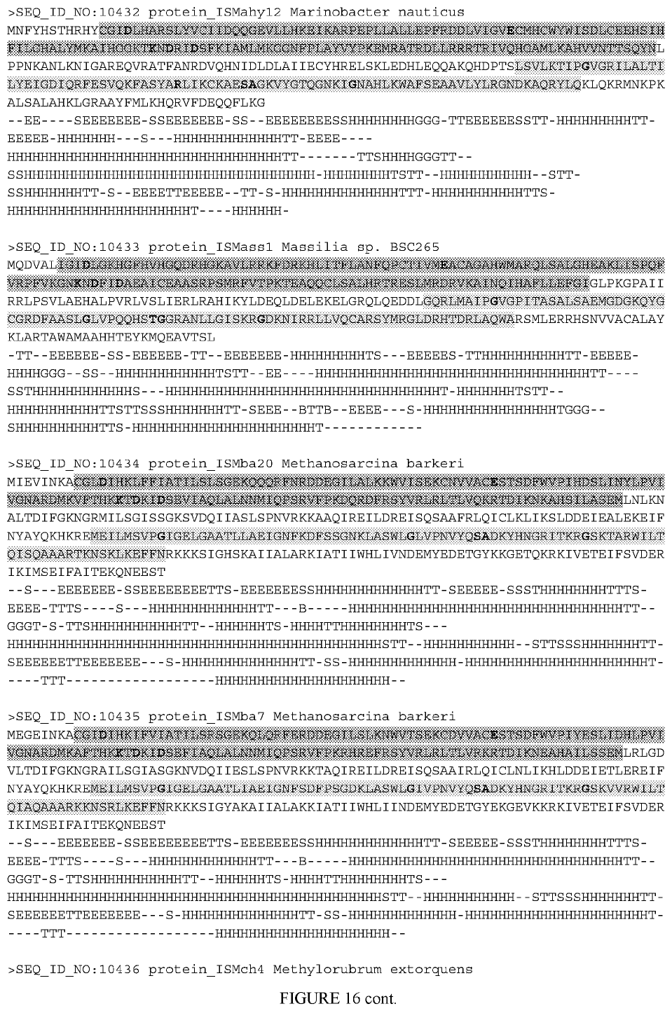

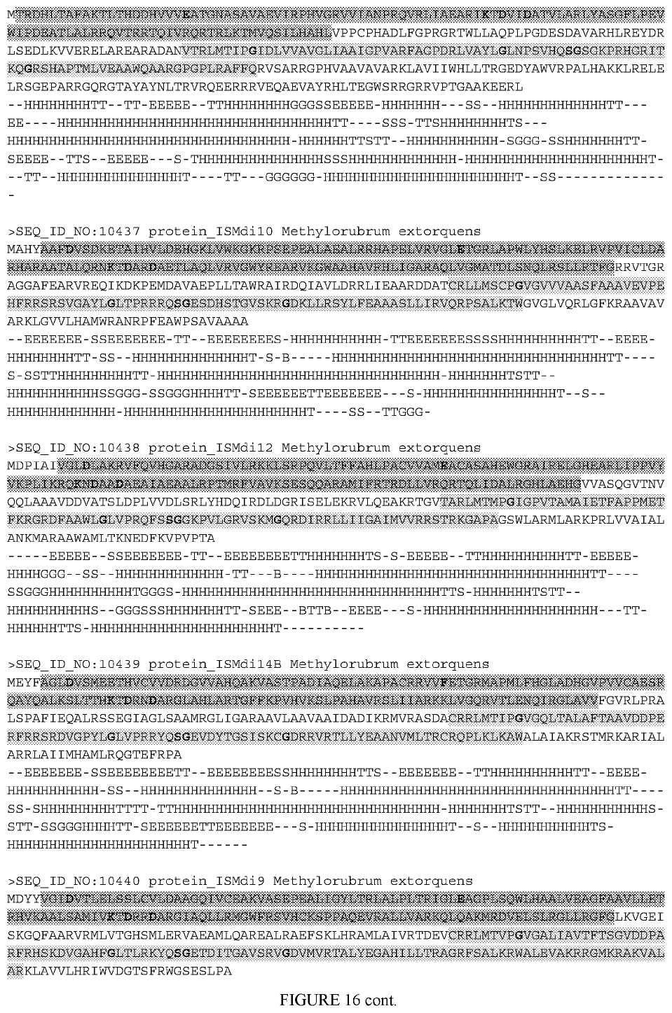

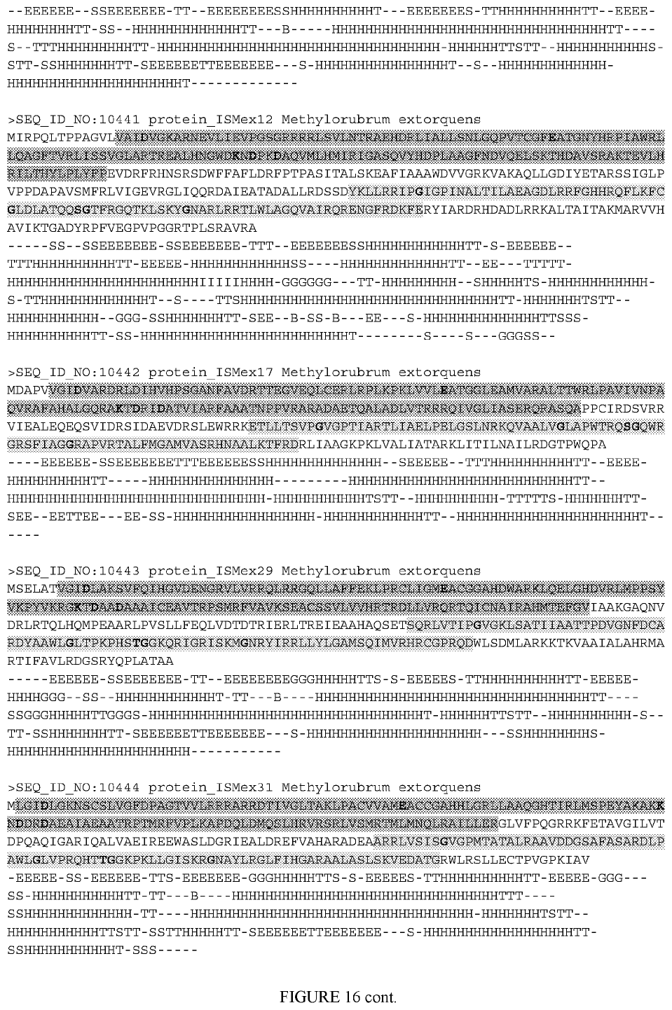

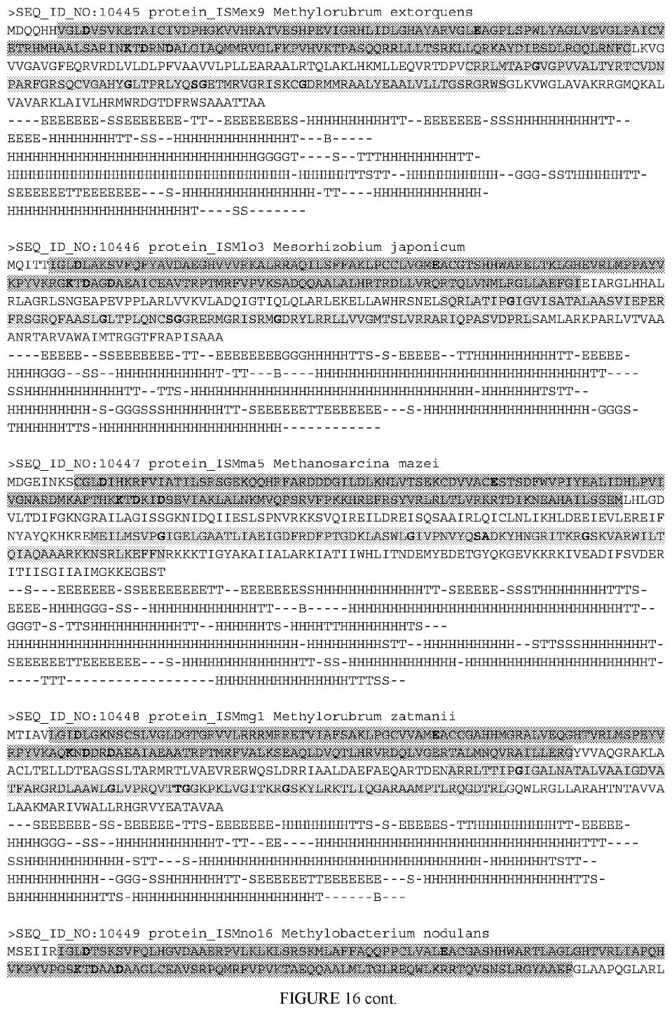

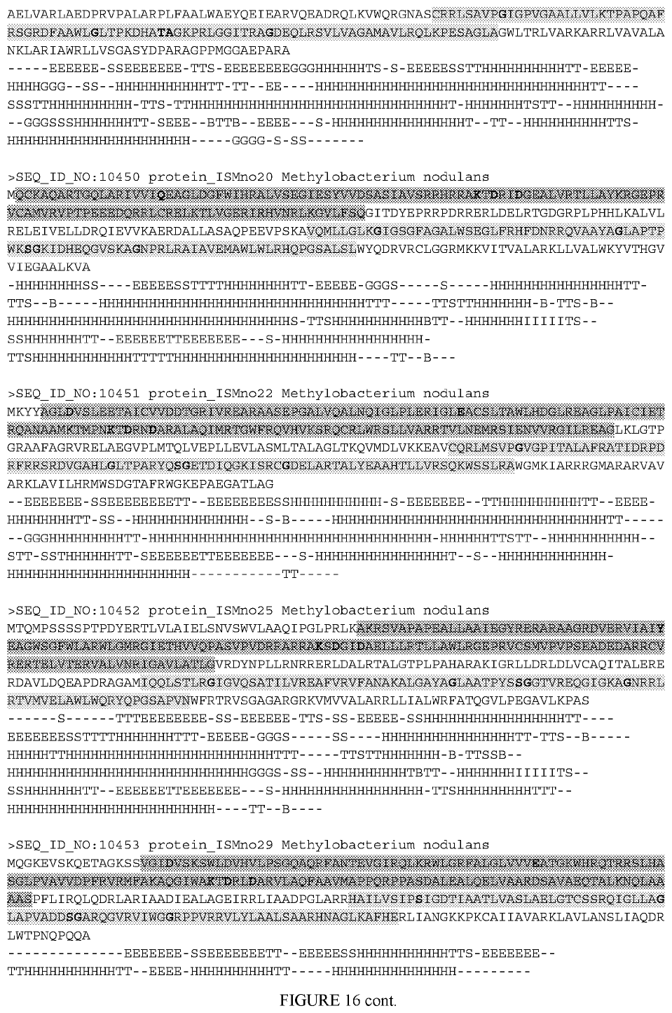

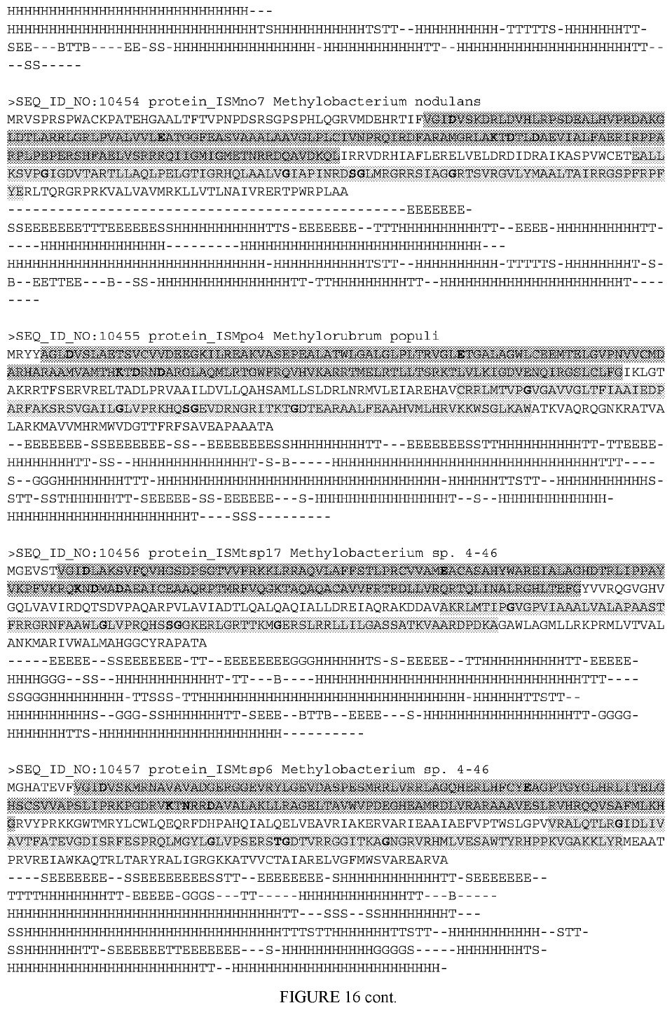

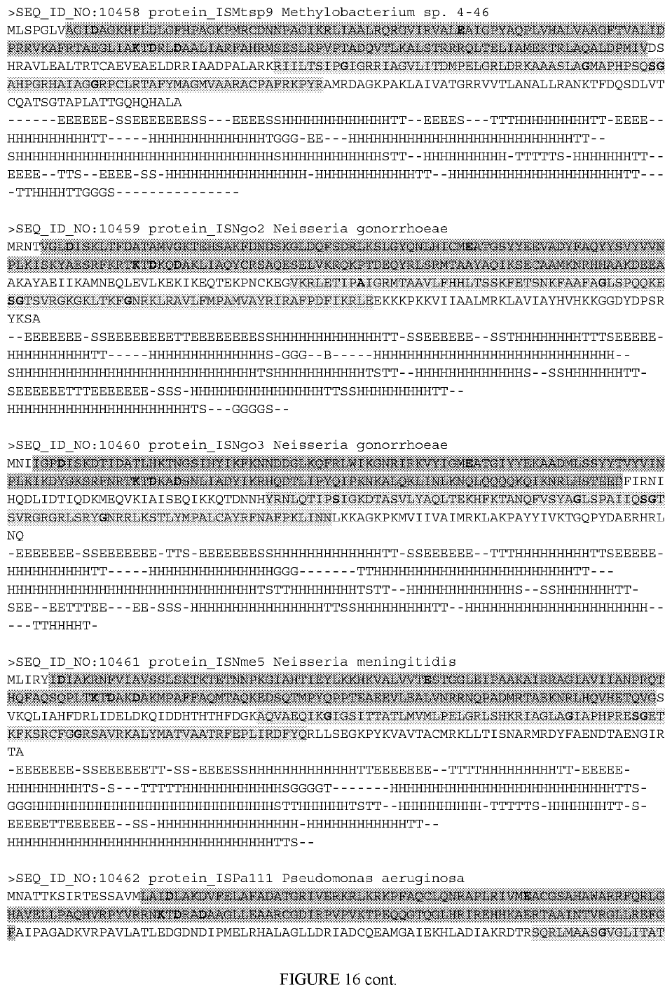

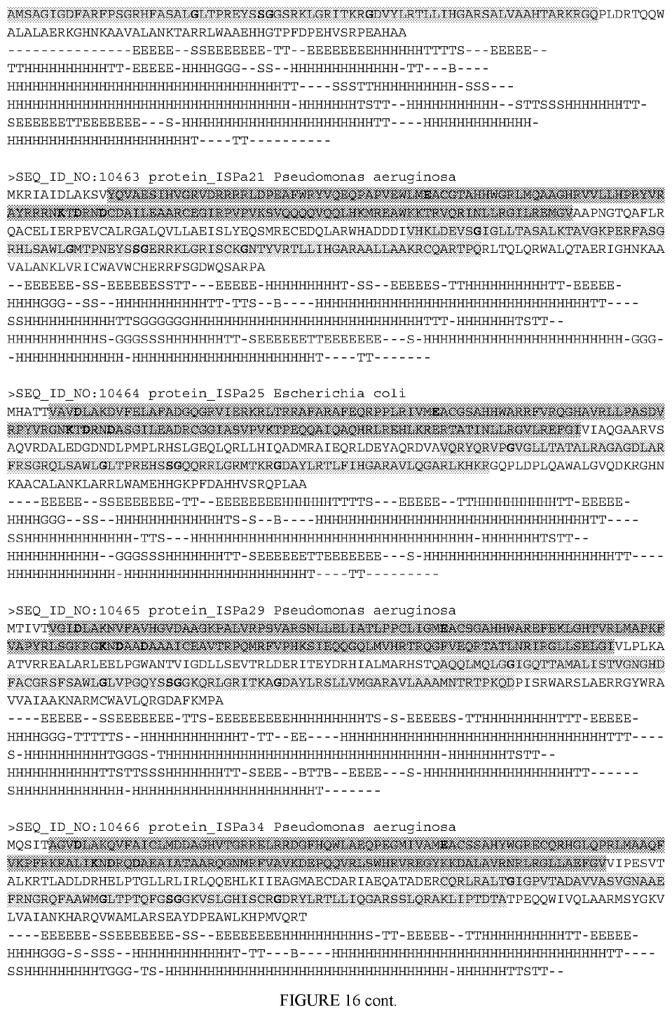

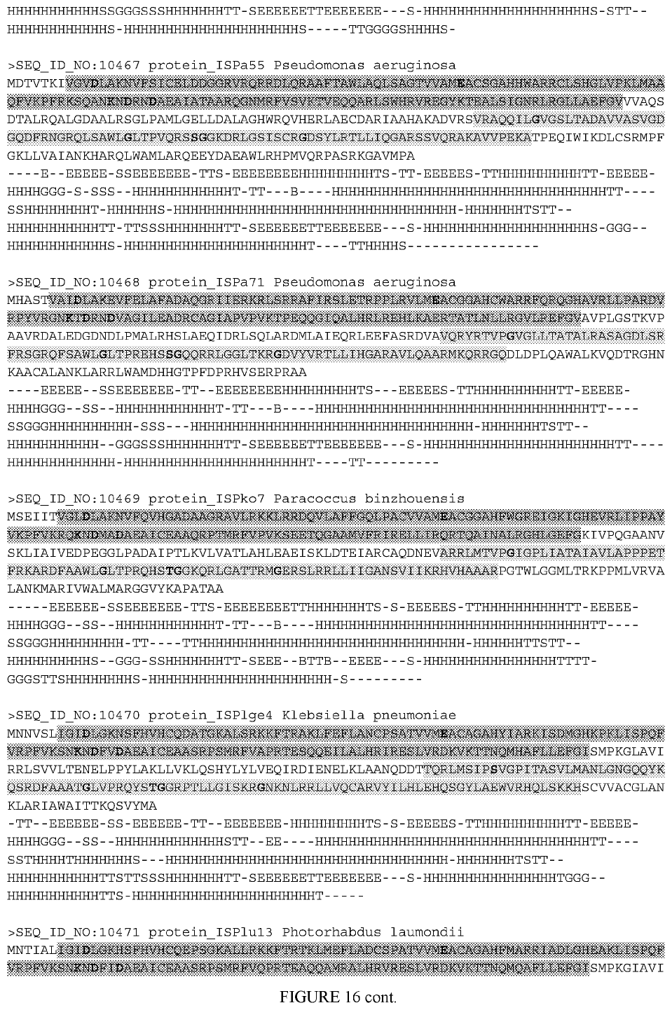

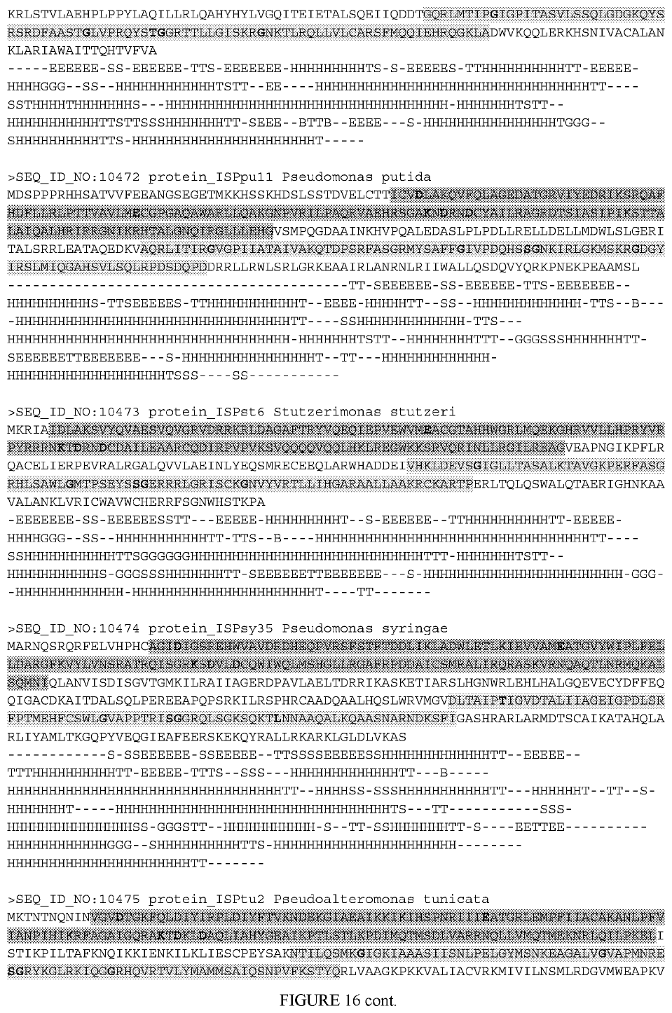

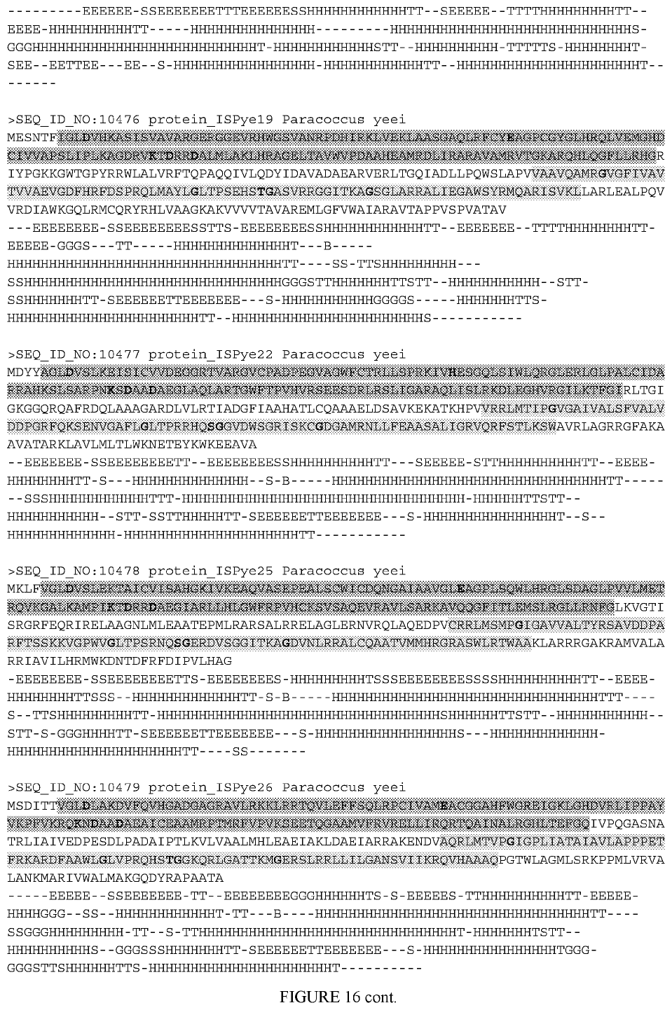

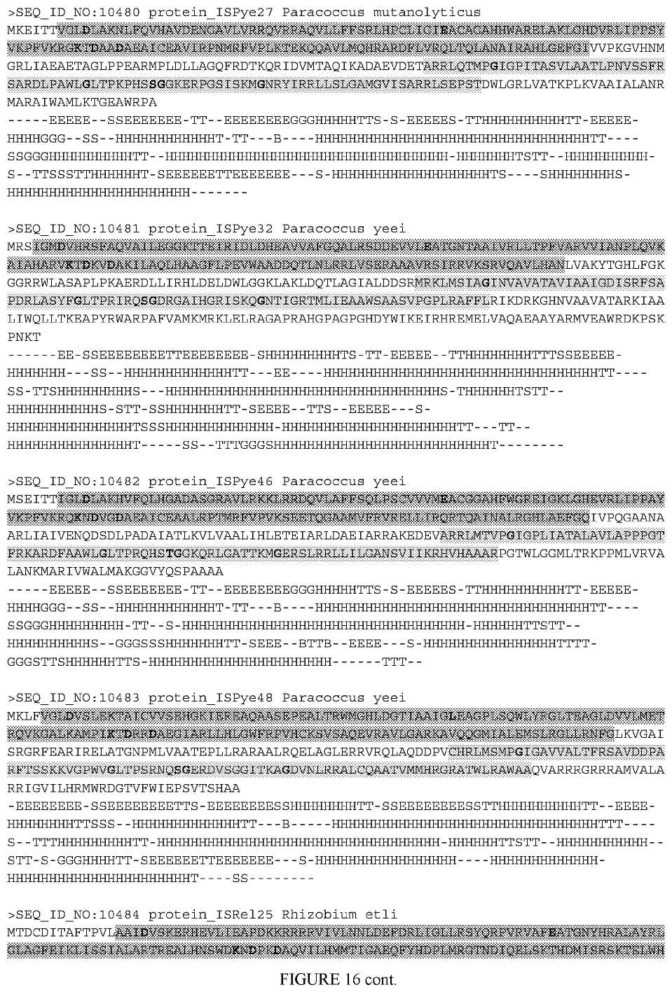

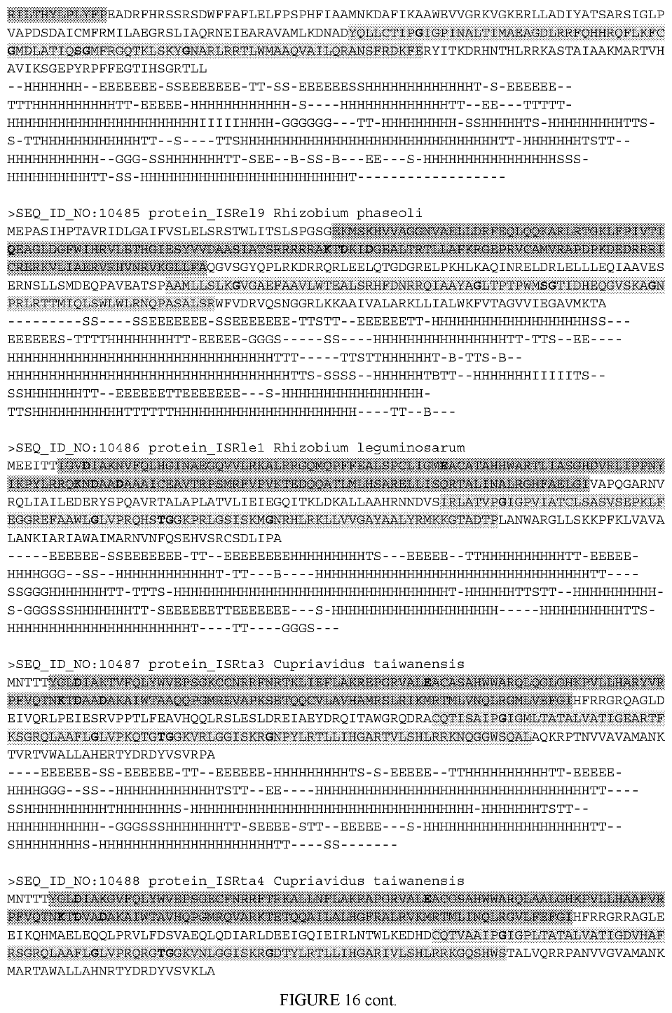

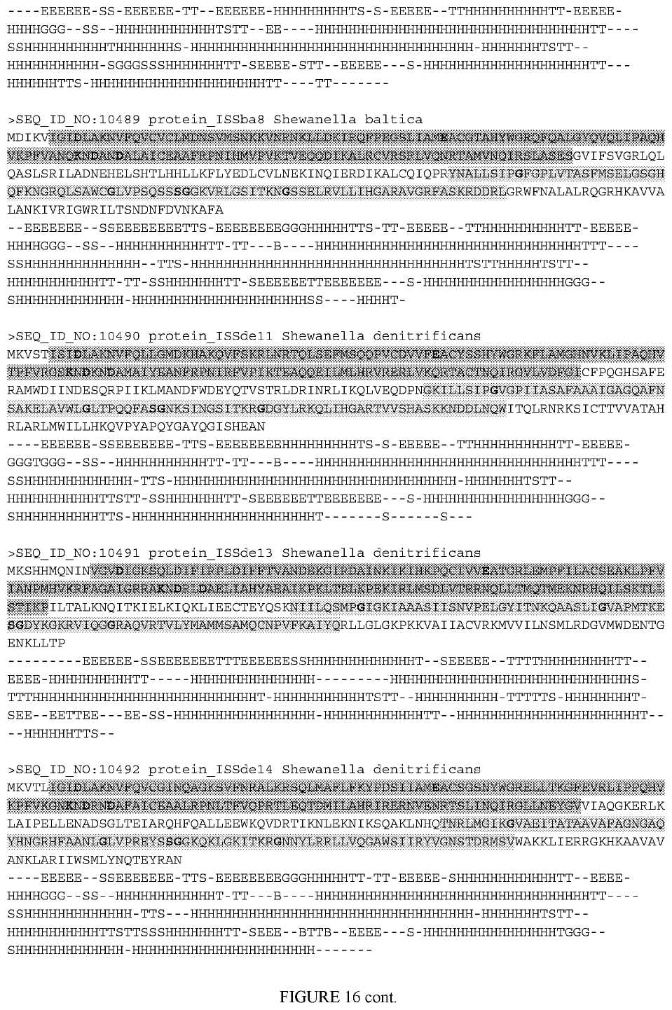

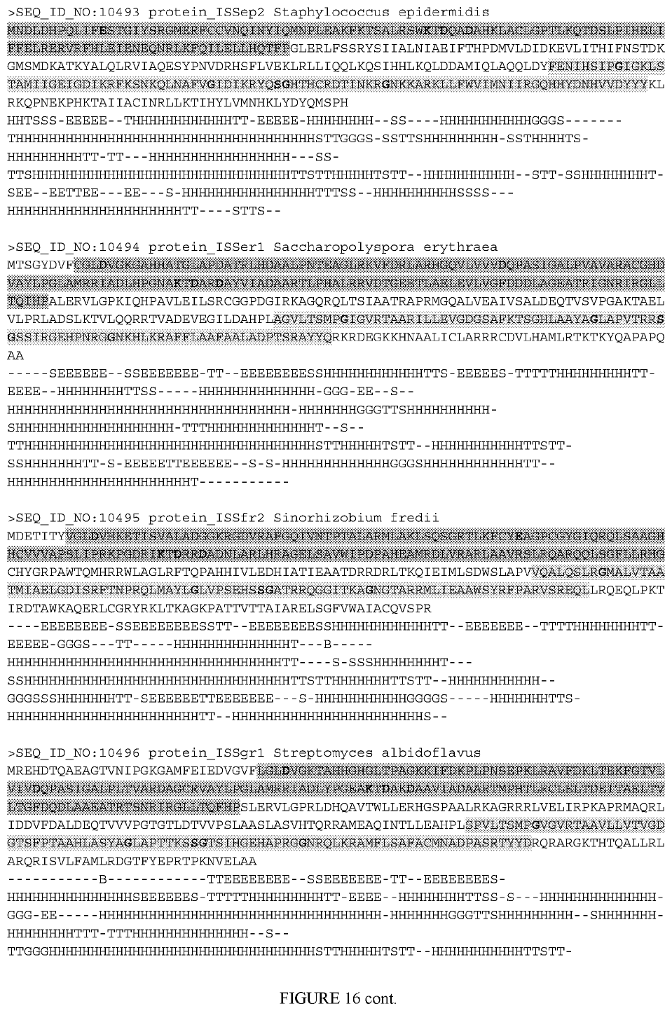

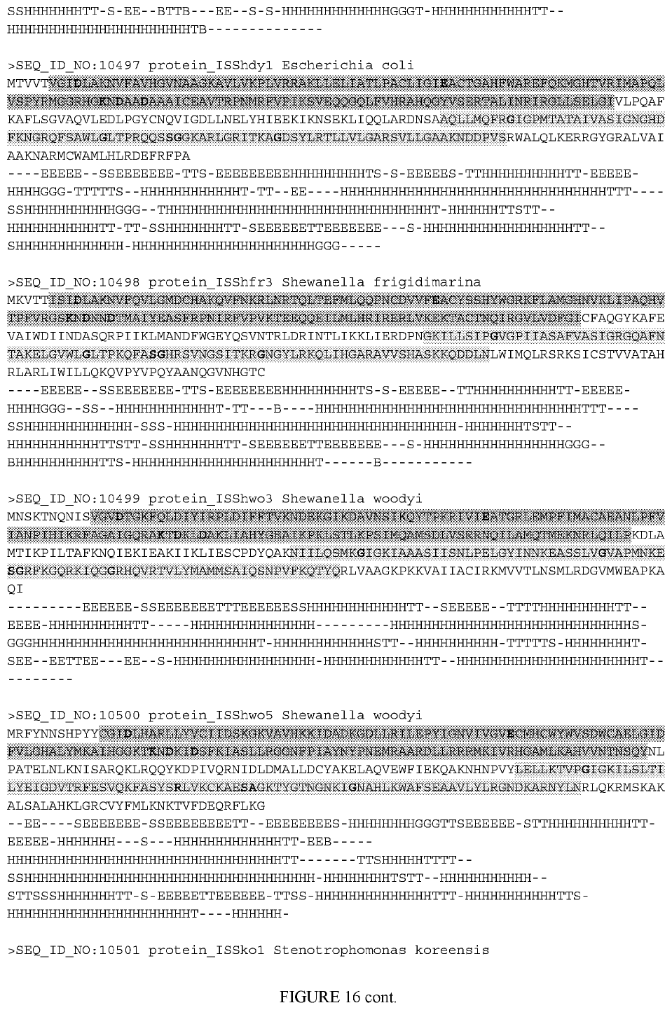

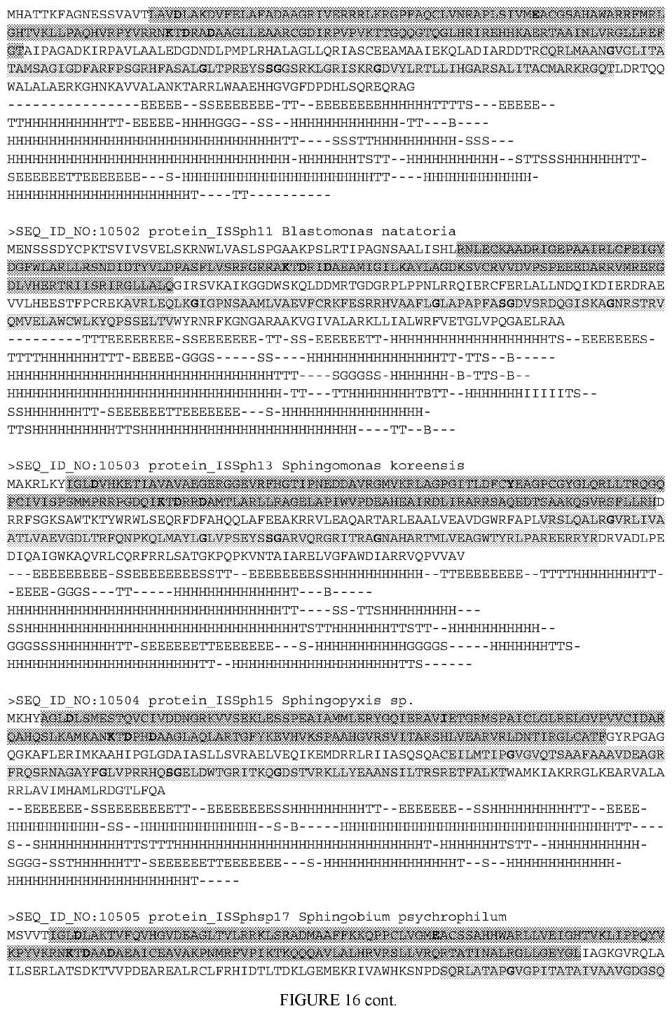

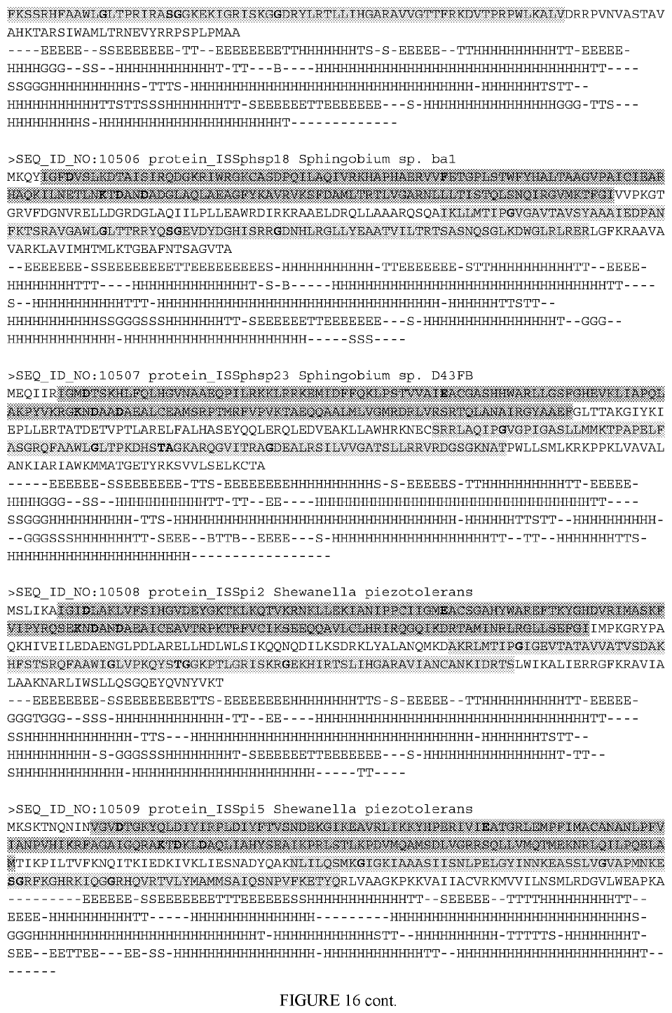

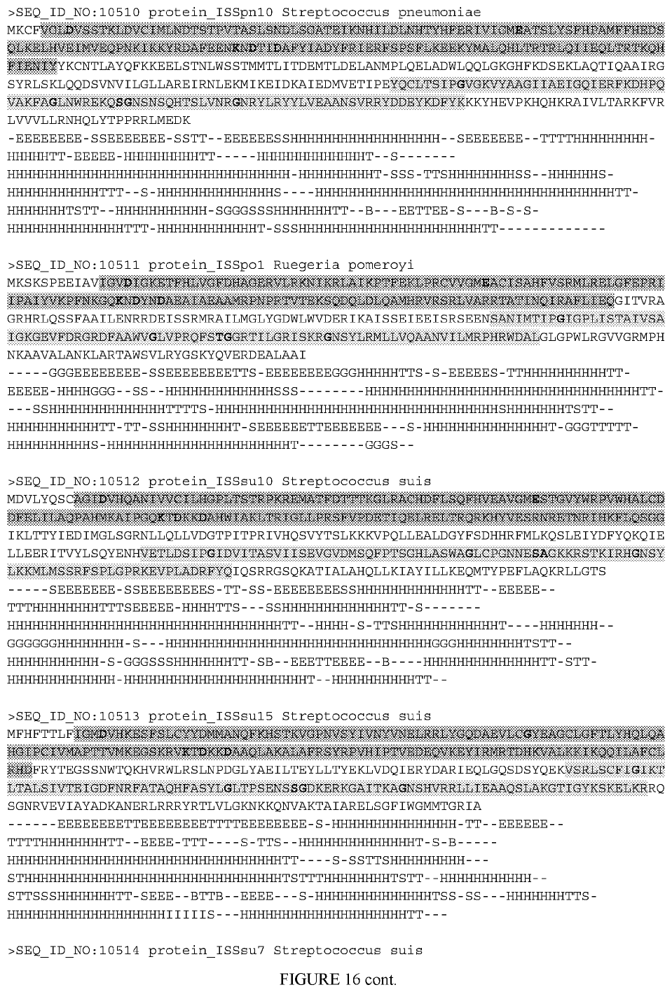

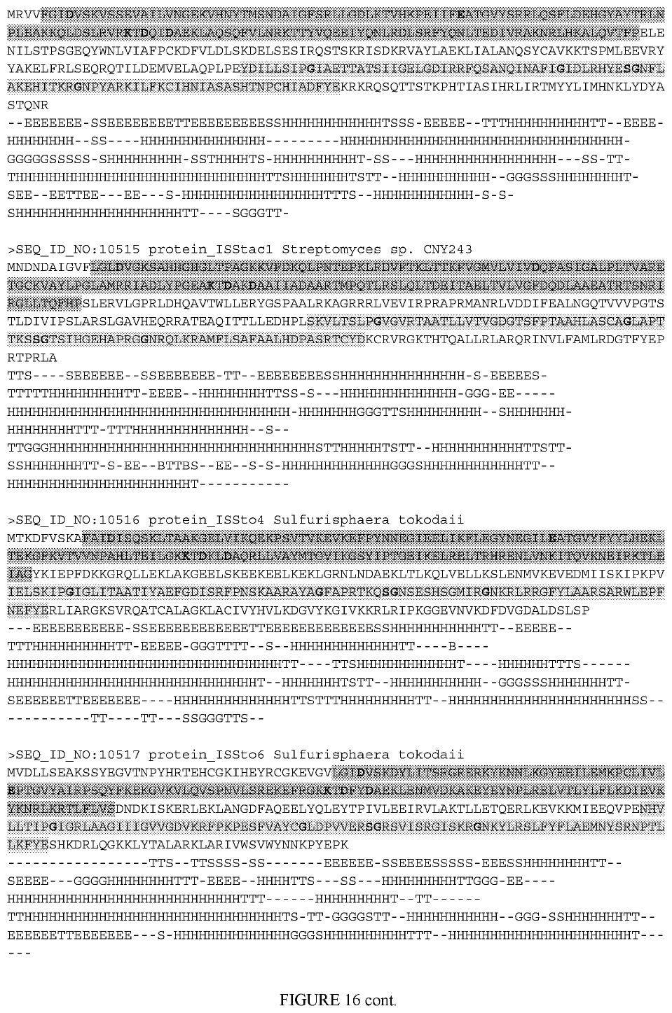

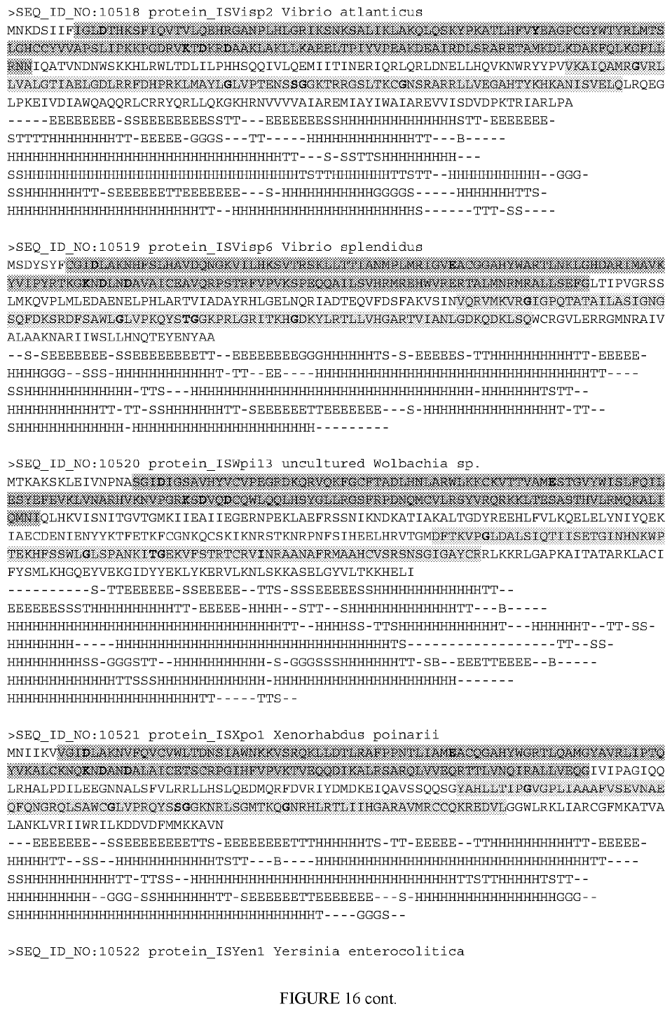

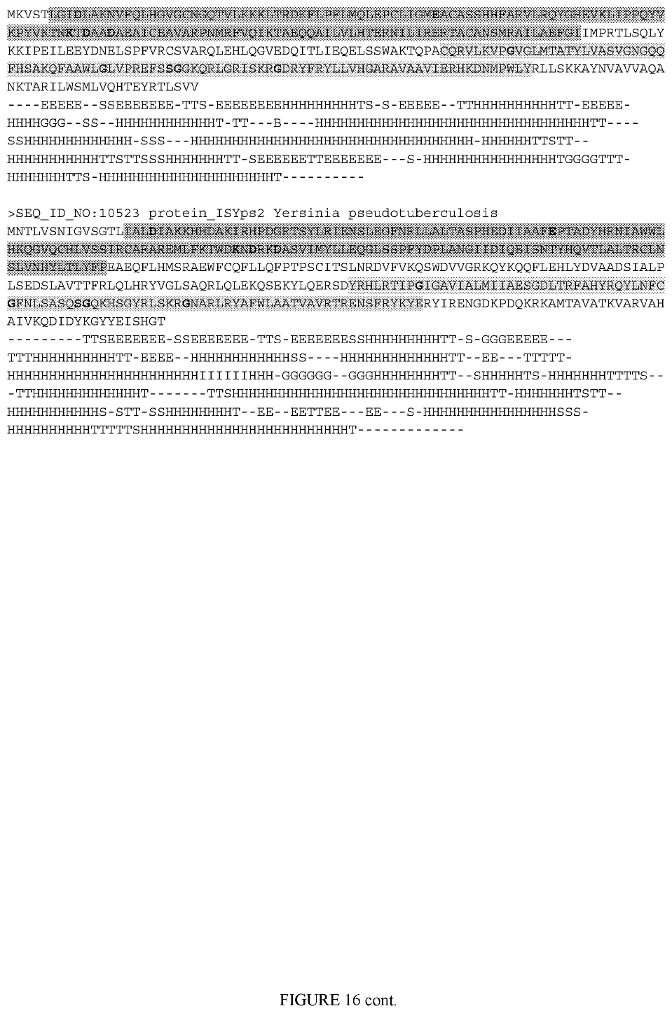

Exemplary primary amino acid sequences and secondary structure prediction of IS110 family transposases are provided in FIG. 16 (SEQ ID NOS: 10176-10523). Additional exemplary primary amino acid sequences of IS110 family transposases are provided in SEQ ID NOS: 10524-20350 and 40357-516430. The RuvC-like DEDD catalytic domain and transposase domain with two canonical Pfam domains are indicated as features of the sequence listing. The polypeptide sequence between the N-terminal RuvC-like DEDD catalytic domain and the C-terminal transposase domain comprises a linker domain comprising a coiled-coil.

In some embodiments, the IS110 family transposase comprises an amino acid sequence that is 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to a sequence provided in FIG. 16 (SEQ ID NOS: 10176-10523) or SEQ ID NOS: 10524-20350 or 40357-516430. In some embodiments the sequence is “protein_IS621” of FIG. 16 (SEQ ID NO:10176).

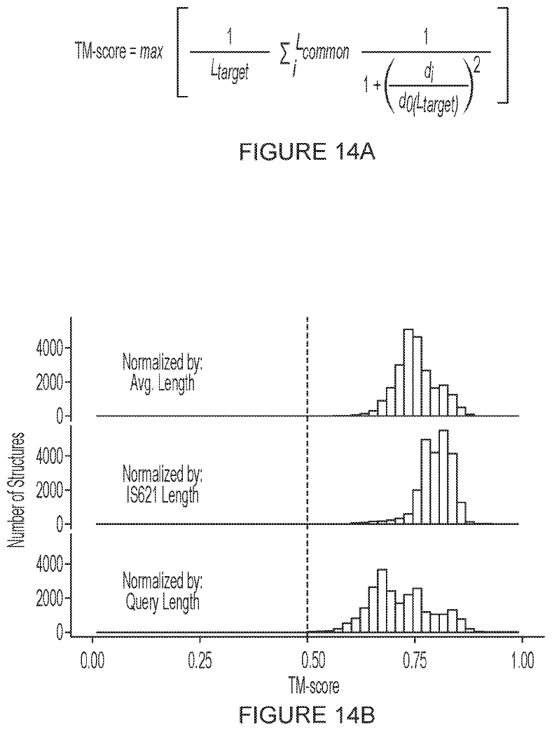

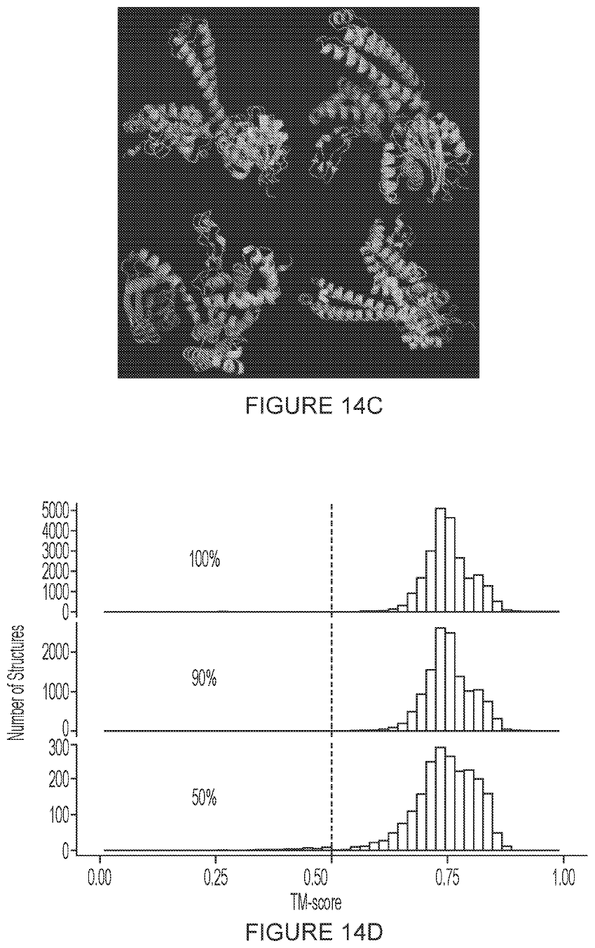

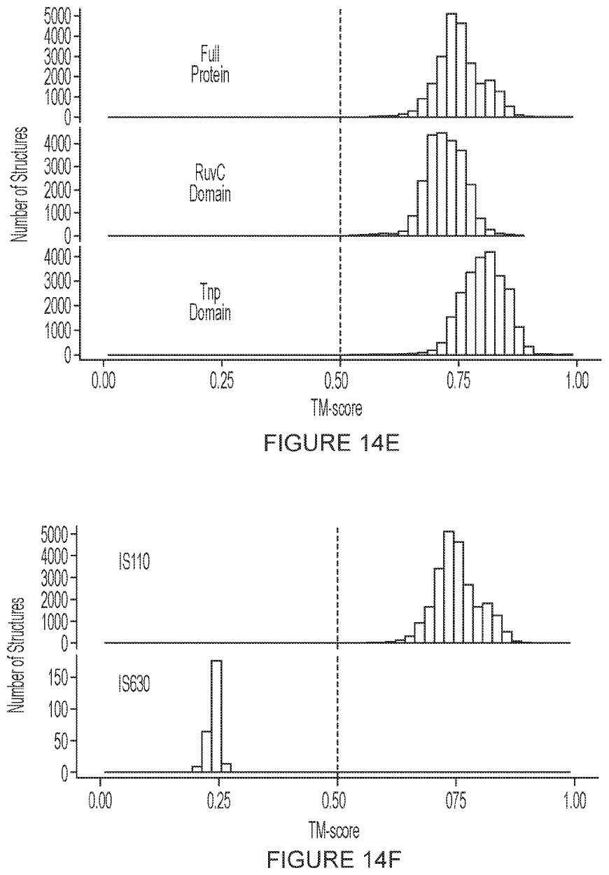

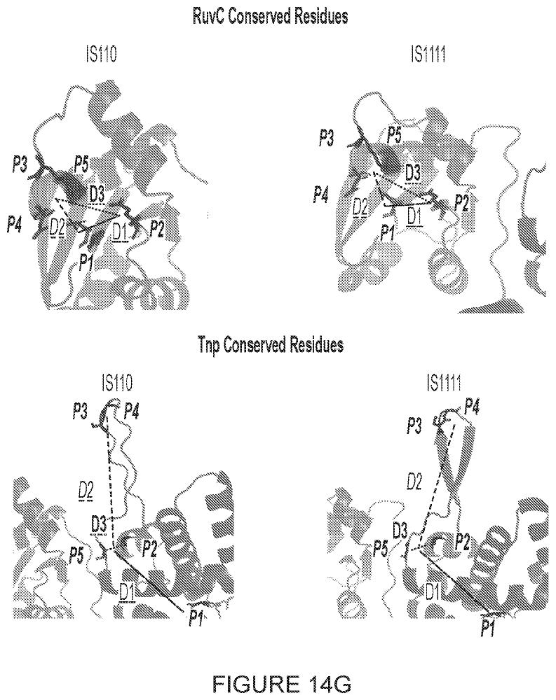

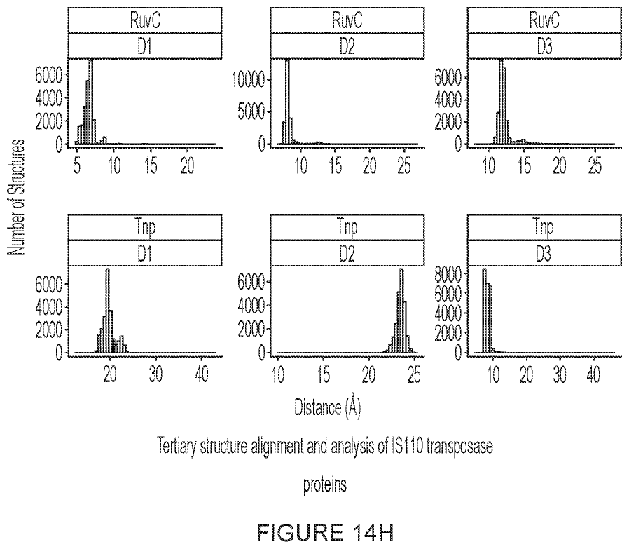

Domain motifs and/or regions of the IS110 family transposase can be identified not necessarily by similarity of amino acid sequences but by structural similarity. In some embodiments, structural similarity is determined by the template modeling score (TM-score). See Example 15 and FIGS. 14A-H. In some embodiments, predicted secondary structure is used to identify domain motifs and/or regions of the IS110 family transposase. In some embodiments, secondary structure of a primary amino acid sequence is predicted using a standard mkdssp tool on tertiary structure files or equivalent protein structure prediction software. In some embodiments, the linker domain of the IS110 family transposase comprises a polypeptide sequence between the RuvC-like DEDD catalytic domain and transposase domain that comprises an amino acid sequence that is predicted to form a coiled-coil.

In some embodiments, the IS110 family transposase comprises a polypeptide that forms a similar tertiary structure to the tertiary structure of IS621 as shown in FIG. 14C. In some embodiments, the tertiary structure is determined using AlphaFold or similar protein structure prediction software. In some embodiments, a particular amino acid sequence is considered to form a similar tertiary structure to that of IS621 if the template modeling score (TM-score) is 0.5 or higher. In some embodiments, a particular amino acid sequence is considered to form a similar tertiary structure to that of IS621 if the template modeling score (TM-score) is 0.5 or higher, 0.6 or higher, 0.7 or higher, 0.8 or higher, or 0.9 or higher.

The TM-score is defined as provided in FIG. 14A, where L_{target} is the length of the amino acid sequence of the target protein, and L_{common} is the number of residues that appear in both the template and target structures. dᵢ is the distance between the ith pair of residues in the template and target structures, and dₒ(L_{target})=1.24−(L_target−15)−1.8 is a distance scale that normalizes distances (Zhang, Yang, and Jeffrey Skolnick. 2004. “Scoring Function for Automated Assessment of Protein Structure Template Quality.” Proteins 57 (4): 702-10). Alternatively, the score can be normalized according to the length of the query protein, or the score can be normalized by the averaged length of the two proteins. A TM-score has a value in (0,1], and a cutoff of >0.5 is commonly used for identifying proteins with homologous tertiary structures (Zhang, Yang, and Jeffrey Skolnick. 2005. “TM-Align: A Protein Structure Alignment Algorithm Based on the TM-Score.” Nucleic Acids Research 33 (7): 2302-9).

In some embodiments, the IS110 family transposase comprises an amino acid sequence that is 15% identical or more, 16% identical or more, 17% identical or more, 18% identical or more, 19% identical or more, 20% identical or more, 21% identical or more, 22% identical or more, 23% identical or more, 24% identical or more, 25% identical or more, 26% identical or more, 27% identical or more, 28% identical or more, 29% identical or more, 30% identical or more, 31% identical or more, 32% identical or more, 33% identical or more, 34% identical or more, 35% identical or more, 36% identical or more, 37% identical or more, 38% identical or more, 39% identical or more, 40% identical or more, 41% identical or more, 42% identical or more, 43% identical or more, 44% identical or more, 45% identical or more, 46% identical or more, 47% identical or more, 48% identical or more, 49% identical or more, 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to a sequence provided in FIG. 16 (SEQ ID NOS: 10176-10523) or SEQ ID NOS: 10524-20350 or 40357-516430 and forms a similar tertiary structure as shown in FIG. 14C. In some embodiments the sequence is “protein_IS621” of FIG. 16 (SEQ ID NO:10176). In some embodiments, the tertiary structure is determined using AlphaFold or similar protein structure prediction software. In some embodiments, a particular amino acid sequence is considered to form a similar tertiary structure to that of IS621 if the template modeling score (TM-score) is 0.5 or higher. In some embodiments, a particular amino acid sequence is considered to form a similar tertiary structure to that of IS621 if the template modeling score (TM-score) is 0.5 or higher, 0.6 or higher, 0.7 or higher, 0.8 or higher, or 0.9 or higher.

In certain aspects, described herein is an IS110 family transposase comprising means for performing a transposase reaction. In some embodiments, the means for performing a transposase reaction comprises a sequence provided in FIG. 16 (SEQ ID NOS: 10176-10523) or SEQ ID NOS: 10524-20350 or 40357-516430.

In certain aspects, described herein are nucleic acids encoding any of the IS110 family transposase amino acid sequences provided herein.

### C.1. RuvC-like DEDD Catalytic Domain

The RuvC-like DEDD catalytic domain refers to the domain of the IS110 transposase that resembles the RuvC Holliday junction resolvase, an abundant protein domain found within proteins of diverse function. The RuvC domain is often found within RNA-guided CRISPR nucleases. RNA-guided RuvC domain bearing CRISPR nucleases are sometimes associated with transposons, such as CRISPR associated transposons (CAST). CRISPR nucleases associated with transposons do not mediate transposition but impart target specificity for the transposome. The IS110 family transposases described herein comprise a RuvC-like DEDD catalytic domain.

In some embodiments, the IS110 family transposase comprises a RuvC-like DEDD catalytic domain comprising an amino acid sequence that is 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the RuvC-like DEDD catalytic domain sequences provided in FIG. 16 (SEQ ID NOS: 10176-10523) or SEQ ID NOS: 10524-20350 or 40357-516430. In some embodiments, the RuvC-like DEDD catalytic domain sequence is the RuvC-like DEDD catalytic domain of “protein_IS621” of FIG. 16.

In some embodiments, the IS110 family transposase comprises a RuvC-like DEDD catalytic domain that forms a similar tertiary structure to the RuvC-like DEDD catalytic domain of IS621. In some embodiments, the tertiary structure is determined using AlphaFold or similar protein structure prediction software. In some embodiments, a particular amino acid sequence is considered to form a similar tertiary structure to the RuvC-like DEDD catalytic domain of IS621 if the template modeling score (TM-score) for the RuvC-like DEDD catalytic domain is 0.5 or higher. In some embodiments, a particular amino acid sequence is considered to form a similar tertiary structure to the RuvC-like DEDD catalytic domain of IS621 if the template modeling score (TM-score) is 0.5 or higher, 0.6 or higher, 0.7 or higher, 0.8 or higher, or 0.9 or higher.

In some embodiments, a particular amino acid sequence is considered to form a similar tertiary structure to the RuvC-like DEDD catalytic domain of IS621 based on distances between the alpha carbon of conserved residues in the RuvC-like DEDD catalytic domain. FIG. 16 (SEQ ID NOS: 10176-10523) provides in bold typeface up to 5 amino acids that are highly conserved in the RuvC-like DEDD catalytic domain. SEQ ID NOS: 10524-20350 or 40357-516430 provide as features P1-P5 of the sequence listing up to 5 amino acids that are highly conserved in the RuvC-like DEDD catalytic domain. Conserved amino acids in a particular amino acid sequence are identified by primary amino acid sequence alignment. In some embodiments, a particular amino acid sequence is considered to form a similar tertiary structure to the RuvC-like DEDD catalytic domain of IS621 if a distance (“D1”) between the alpha carbon of a first conserved residue and the alpha carbon of a second conserved residue of the amino acid sequence is less than 10 angstroms (Å), wherein the conserved residues of the amino acid sequence is per alignment of the primary amino acid sequence with one or more RuvC-like DEDD catalytic domains, such as IS621. In IS621 the first conserved residue is D11 and the second conserved residue is E60. In some embodiments, D1 is between 4 and 10 angstroms (Å). In some embodiments, D1 is between 5 and 7.5 angstroms (Å). In some embodiments, a particular amino acid sequence is considered to form a similar tertiary structure to the RuvC-like DEDD catalytic domain of IS621 if an average distance (“D2”) between the alpha carbon of a first conserved residue and the alpha carbon of a third conserved residue, between the alpha carbon of a first conserved residue and the alpha carbon of a fourth conserved residue, and between the alpha carbon of a first conserved residue and the alpha carbon of a fifth conserved residue, is less than 10 angstroms (Å), wherein the conserved residues of the amino acid sequence is per alignment of the primary amino acid sequence with one or more RuvC-like DEDD catalytic domains, such as IS621. In IS621 the first conserved residue is D11, the third conserved residue is K100, the fourth conserved residue is D102, and the fifth conserved residue is D105. In some embodiments, D2 is between 5 and 10 angstroms (Å). In some embodiments, D2 is between 7.5 and 10 angstroms (Å). In some embodiments, a particular amino acid sequence is considered to form a similar tertiary structure to the RuvC-like DEDD catalytic domain of IS621 if an average distance (“D3”) between the alpha carbon of a second conserved residue and the alpha carbon of a third conserved residue, between the alpha carbon of a second conserved residue and the alpha carbon of a fourth conserved residue, and between the alpha carbon of a second conserved residue and the alpha carbon of a fifth conserved residue, is less than 15 angstroms (Å), wherein the conserved residues of the amino acid sequence is per alignment of the primary amino acid sequence with one or more RuvC-like DEDD catalytic domains, such as IS621. In IS621 the second conserved residue is E60, the third conserved residue is K100, the fourth conserved residue is D102, and the fifth conserved residue is D105. In some embodiments, D3 is between 10 and 15 angstroms (Å). In some embodiments, D3 is between 13 and 15 angstroms (Å). In some embodiments, a particular amino acid sequence is considered to form a similar tertiary structure to the RuvC-like DEDD catalytic domain of IS621 if D1 is less than 10 angstroms (Å), D2 is less than 10 angstroms (Å), and D3 is less than 15 angstroms (Å). In some embodiments, a particular amino acid sequence is considered to form a similar tertiary structure to the RuvC-like DEDD catalytic domain of IS621 if D1 is between 4 and 10 angstroms (Å), D2 is between 5 and 10 angstroms (Å), and D3 is between 10 and 15 angstroms (Å). In some embodiments, a particular amino acid sequence is considered to form a similar tertiary structure to the RuvC-like DEDD catalytic domain of IS621 if D1 is between 5 and 7.5 angstroms (Å), D2 is between 7.5 and 10 angstroms (Å), and D3 is between 13 and 15 angstroms (Å).

In some embodiments, the IS110 family transposase comprises a RuvC-like DEDD catalytic domain comprising an amino acid sequence that is 15% identical or more, 16% identical or more, 17% identical or more, 18% identical or more, 19% identical or more, 20% identical or more, 21% identical or more, 22% identical or more, 23% identical or more, 24% identical or more, 25% identical or more, 26% identical or more, 27% identical or more, 28% identical or more, 29% identical or more, 30% identical or more, 31% identical or more, 32% identical or more, 33% identical or more, 34% identical or more, 35% identical or more, 36% identical or more, 37% identical or more, 38% identical or more, 39% identical or more, 40% identical or more, 41% identical or more, 42% identical or more, 43% identical or more, 44% identical or more, 45% identical or more, 46% identical or more, 47% identical or more, 48% identical or more, 49% identical or more, 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the RuvC-like DEDD catalytic domain sequences provided in FIG. 16 (SEQ ID NOS: 10176-10523) or SEQ ID NOS: 10524-20350 or 40357-516430 and forms a similar tertiary structure to the RuvC-like DEDD catalytic domain of IS621. In some embodiments, the tertiary structure is determined using AlphaFold or similar protein structure prediction software. In some embodiments, a particular amino acid sequence is considered to form a similar tertiary structure to the RuvC-like DEDD catalytic domain of IS621 if the template modeling score (TM-score) for the RuvC-like DEDD catalytic domain is 0.5 or higher. In some embodiments, a particular amino acid sequence is considered to form a similar tertiary structure to the RuvC-like DEDD catalytic domain of IS621 if the template modeling score (TM-score) is 0.5 or higher, 0.6 or higher, 0.7 or higher, 0.8 or higher, or 0.9 or higher. In some embodiments the RuvC-like DEDD catalytic domain sequence provided is the RuvC-like DEDD catalytic domain of “protein_IS621” of FIG. 16 (SEQ ID NO:10176).

In some embodiments, the RuvC-like DEDD catalytic domain can be identified using statistical models that annotate protein domains, such as Pfam profile hidden markov models (pHMMs). In IS110 family transposases, the RuvC-like DEDD catalytic domain is often recognized by the Pfam profile hidden markov model (pHMM) PF01548, short name DEDD_Tnp_IS110.

In some embodiments, the IS110 family transposase comprises a RuvC-like DEDD catalytic domain comprising a motif D-x(43)-E-x(39)-K-x(1)-D-x(2)-D (SEQ ID NO: 795142), D-x(42)-E-x(34)-K-x(1)-D-x(2)-D (SEQ ID NO: 795143), [DE]-x(38,63)-[EACDGQVIPS]-x(30,53)-[KSQIVRHLTMA]-x(1)-[DNE]-x(2)-[DEASCM], [DE]-x(41,59)-[EYALGHCVFITMS]-x(30,45)-[KRMQNH]-x(1)-[DN]-x(2)-[DAS], GIDVS (SEQ ID NO: 795144), GLDVH (SEQ ID NO: 795145), [GAS][ILVDFMACWTHGN][DE][VTIWPLAFCSMR][SAHGCD], [GAS][LIMVCFA]D[VLIFAYQTHMDCWRGS][HASGD], MEATG (SEQ ID NO: 795146), MEACG (SEQ ID NO: 795147), [MLVIFCYAGTWSHPR]-x(0,2)-[EGACQDVIMP][ASGPHIDRYLNQCTVFMEK][TSECPAYGIVDNFKLMHR]-x(0,1)-[GASTRDQVNLWEKYHICMP], [MYVCLIASFTGQEHW]-x(0,2)-[EYQGVACFLHITMSDKW]-x(0,2)-[AEVYSFMGTICNPLDQ]-x(0,2)-[CGATESMVLINPFDQW]-x(0,2)-[GPASTLCYINRMVFEWHQDK], DRIDA (SEQ ID NO: 795148), DRRDA (SEQ ID NO: 795149), [DNE][RPAKVTSQEFGIDLHMNYWC][ILKVAGTNRSFQPHDEMCWY][DAESCM][A CSPLTGV], or [DN][RAKEYDQGFVPTMSLHWNIC][RALNIVKHTQDSEMGWFYCP][DSA][ASTGCV LI],with motifs in common Prosite format, where x is any amino acid and x(n) represents n number of any amino acid and x(n,m) represents n to m number of any amino acids. In some embodiments, the RuvC-like DEDD catalytic domain comprises a motif D-x(43)-E-x(39)-K-x(1)-D-x(2)-D (SEQ ID NO: 795142), D-x(42)-E-x(34)-K-x(1)-D-x(2)-D (SEQ ID NO: 795143), GIDVS (SEQ ID NO: 795144), GLDVH (SEQ ID NO: 795145), MEATG (SEQ ID NO: 795146), MEACG (SEQ ID NO: 795147), DRIDA (SEQ ID NO: 795148), or DRRDA (SEQ ID NO: 795149). In some embodiments, the RuvC-like DEDD catalytic domain comprising a motif above forms a similar tertiary structure to the RuvC-like DEDD catalytic domain of IS621. In some embodiments, the tertiary structure is determined using AlphaFold or similar protein structure prediction software. In some embodiments, a particular amino acid sequence is considered to form a similar tertiary structure to the RuvC-like DEDD catalytic domain of IS621 if the template modeling score (TM-score) for the RuvC-like DEDD catalytic domain is 0.5 or higher. In some embodiments, a particular amino acid sequence is considered to form a similar tertiary structure to the RuvC-like DEDD catalytic domain of IS621 if the template modeling score (TM-score) is 0.5 or higher, 0.6 or higher, 0.7 or higher, 0.8 or higher, or 0.9 or higher.

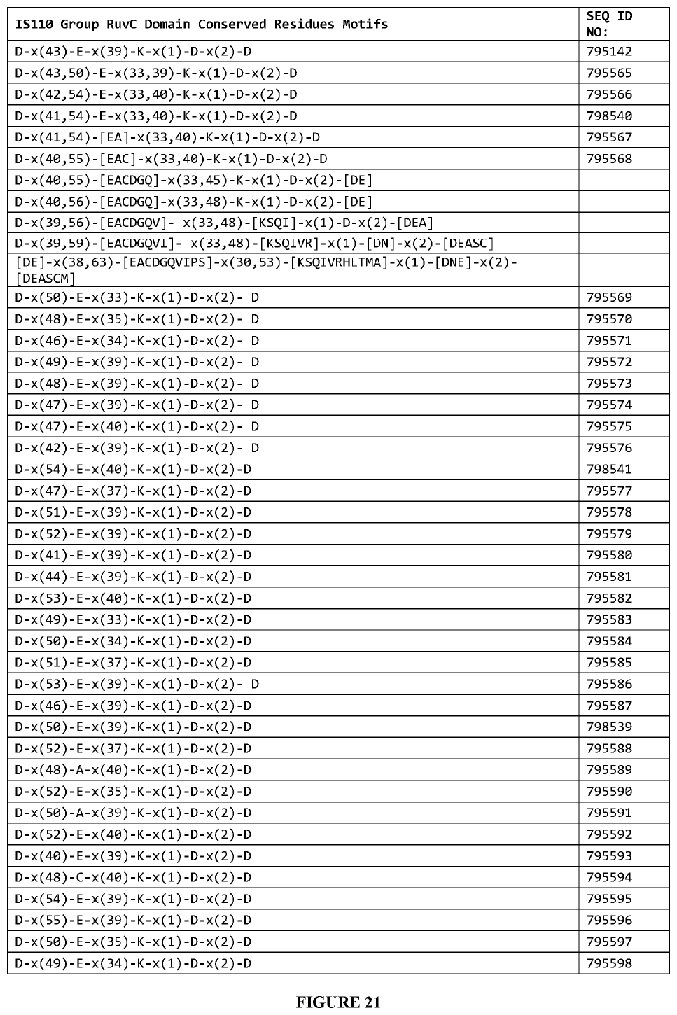

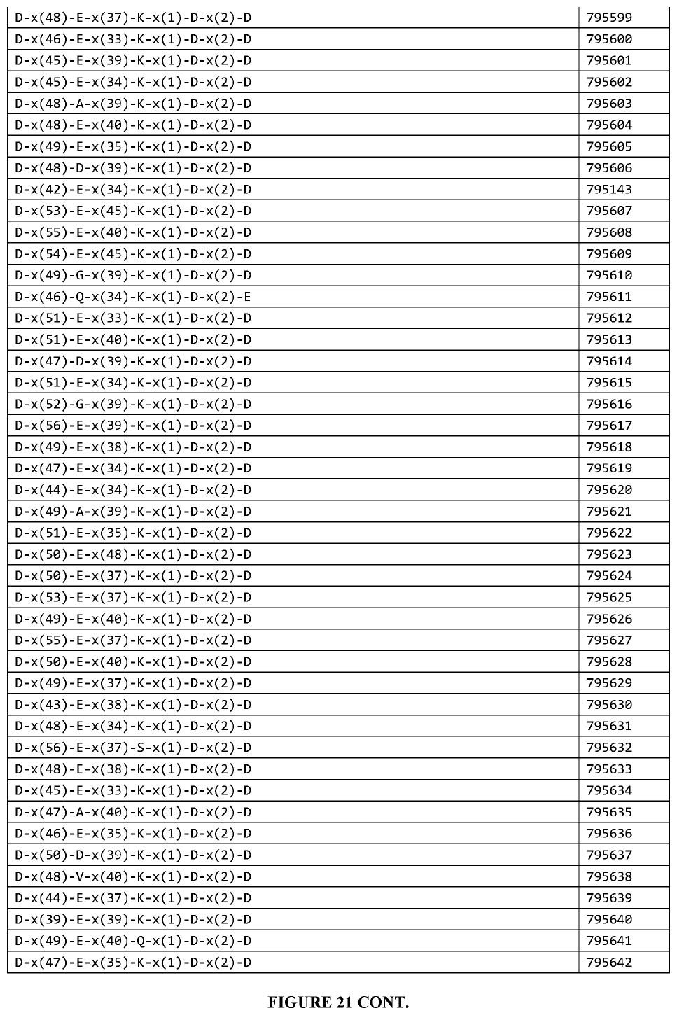

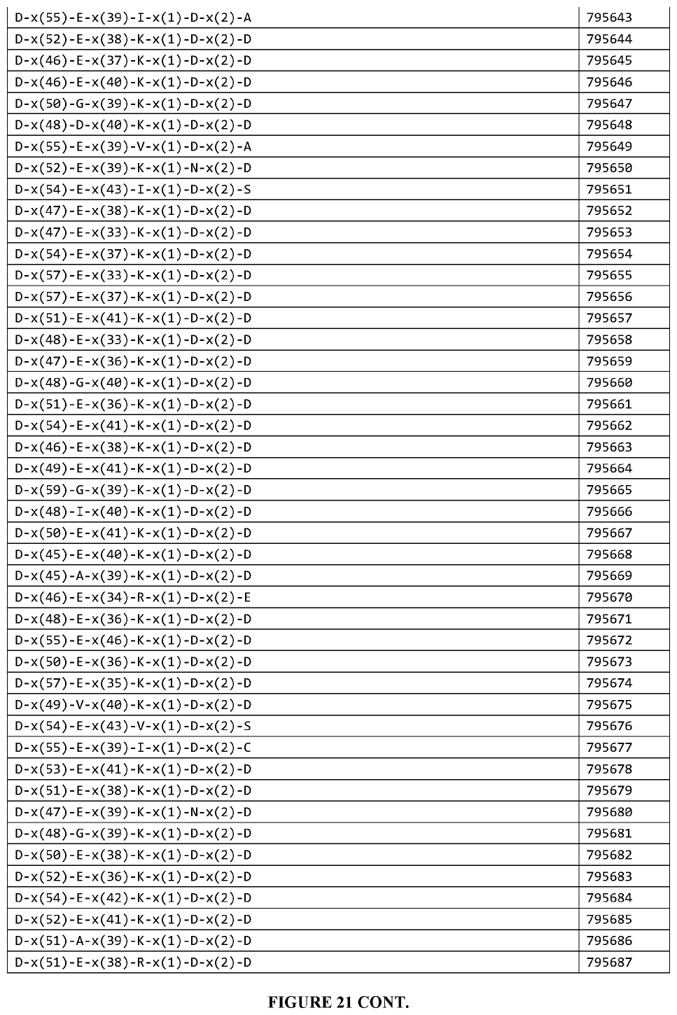

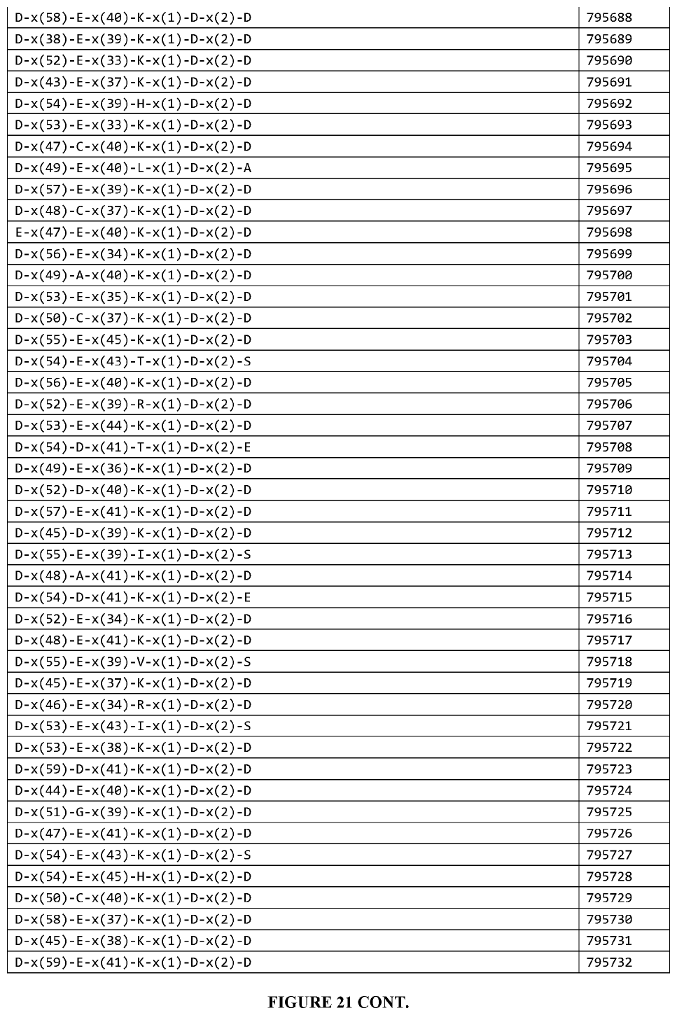

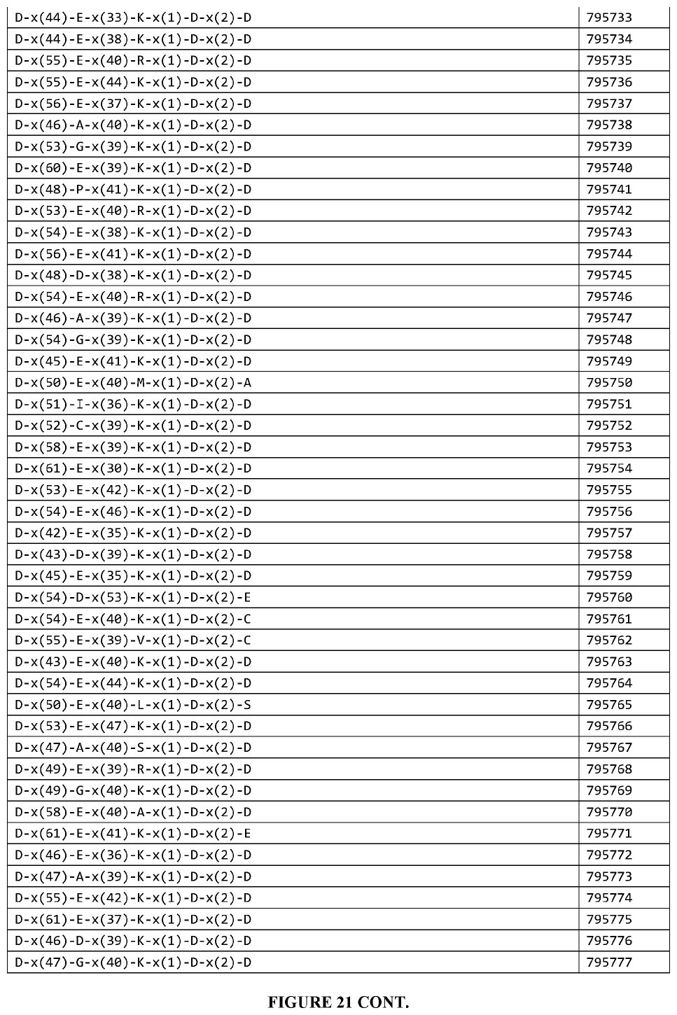

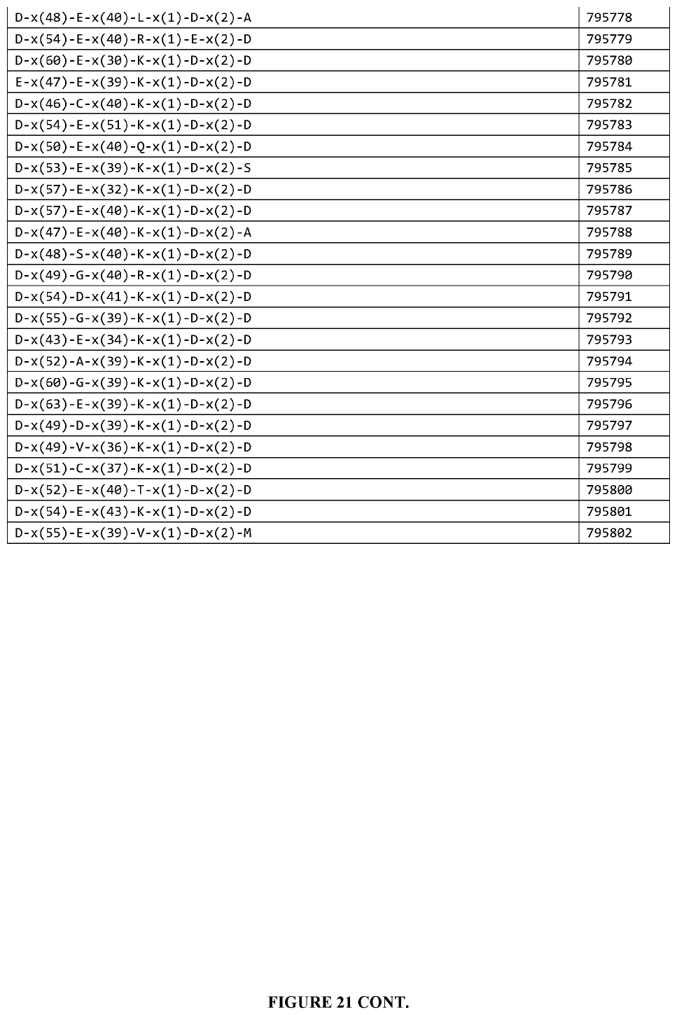

In some embodiments, the IS110 family transposase comprises a RuvC-like DEDD catalytic domain comprising an amino acid sequence that is 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to a RuvC-like DEDD catalytic domain sequence of FIG. 16 (SEQ ID NOS: 10176-10523) or SEQ ID NOS: 10524-20350 or 40357-516430 and comprising any of the motifs in the preceding paragraph or in FIGS. 21-28. In some embodiments, the amino acid sequence forms a similar tertiary structure to the RuvC-like DEDD catalytic domain of IS621. In some embodiments, the tertiary structure is determined using AlphaFold or similar protein structure prediction software. In some embodiments, a particular amino acid sequence is considered to form a similar tertiary structure to the RuvC-like DEDD catalytic domain of IS621 if the template modeling score (TM-score) for the RuvC-like DEDD catalytic domain is 0.5 or higher. In some embodiments, a particular amino acid sequence is considered to form a similar tertiary structure to the RuvC-like DEDD catalytic domain of IS621 if the template modeling score (TM-score) is 0.5 or higher, 0.6 or higher, 0.7 or higher, 0.8 or higher, or 0.9 or higher.

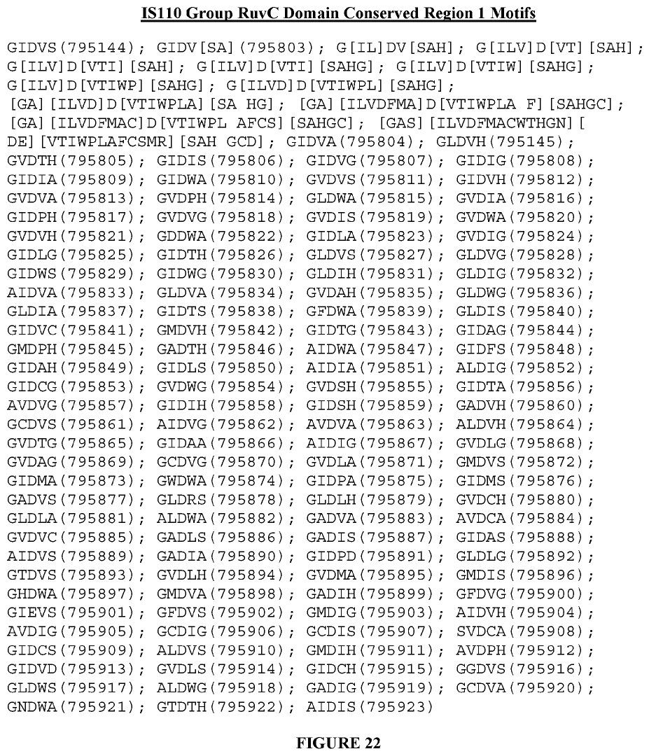

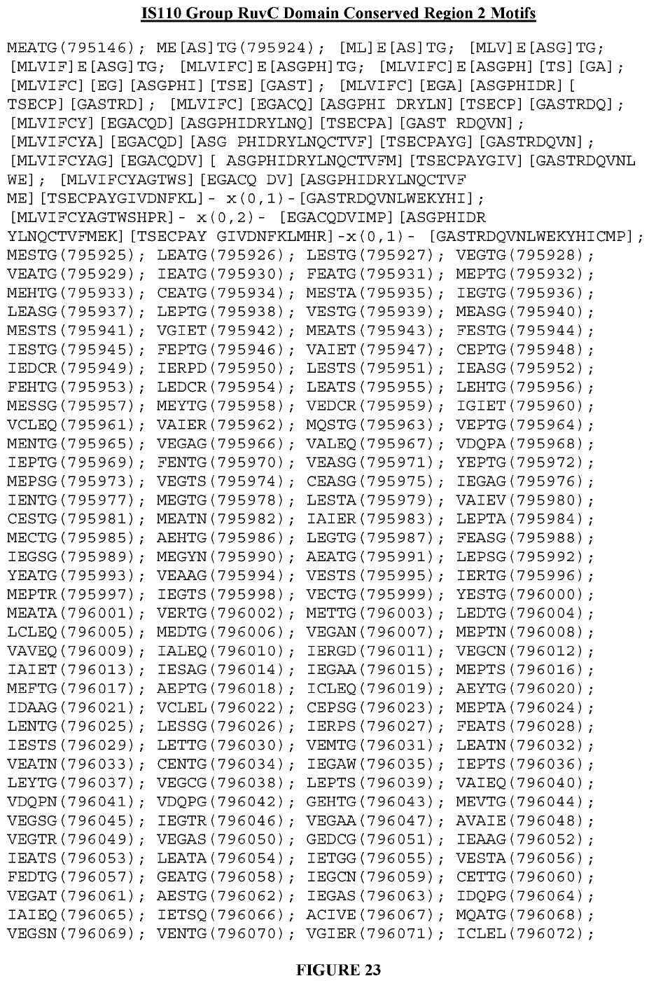

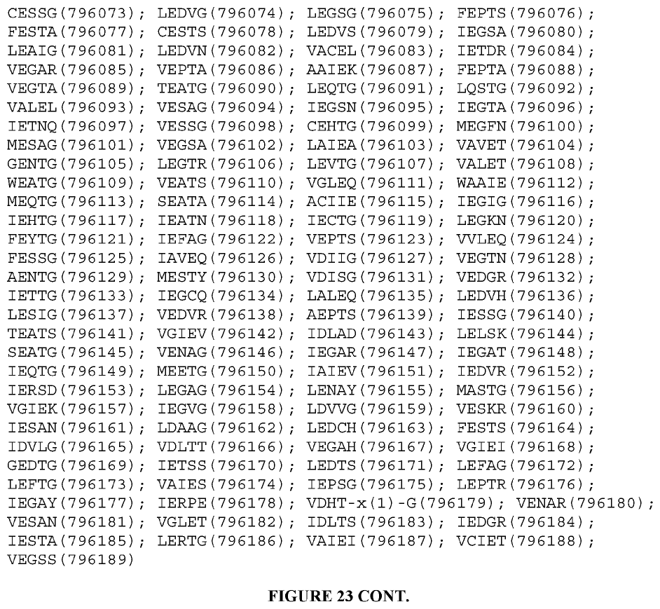

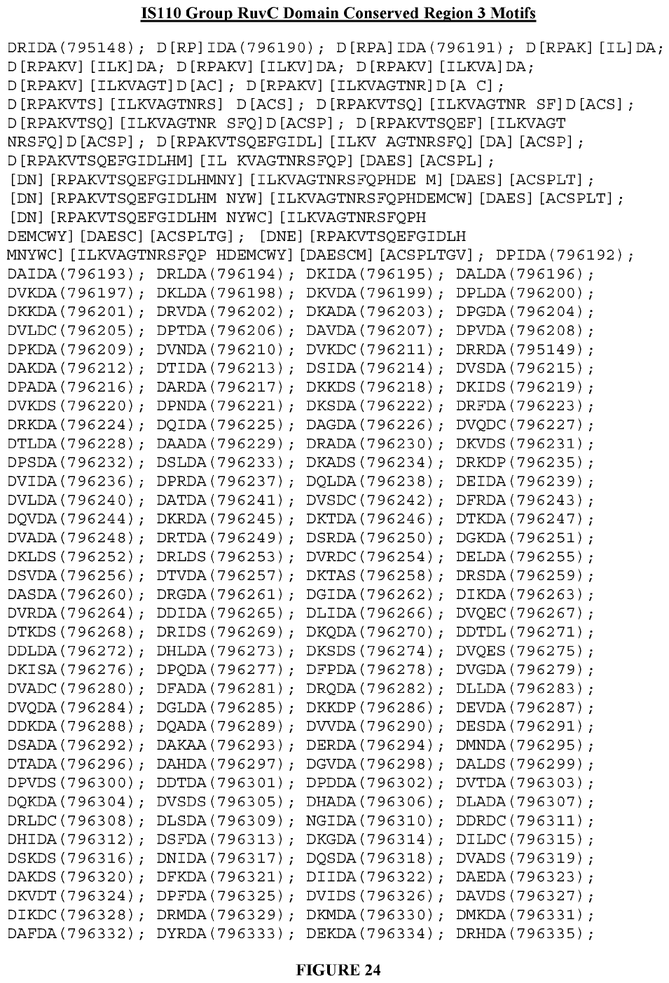

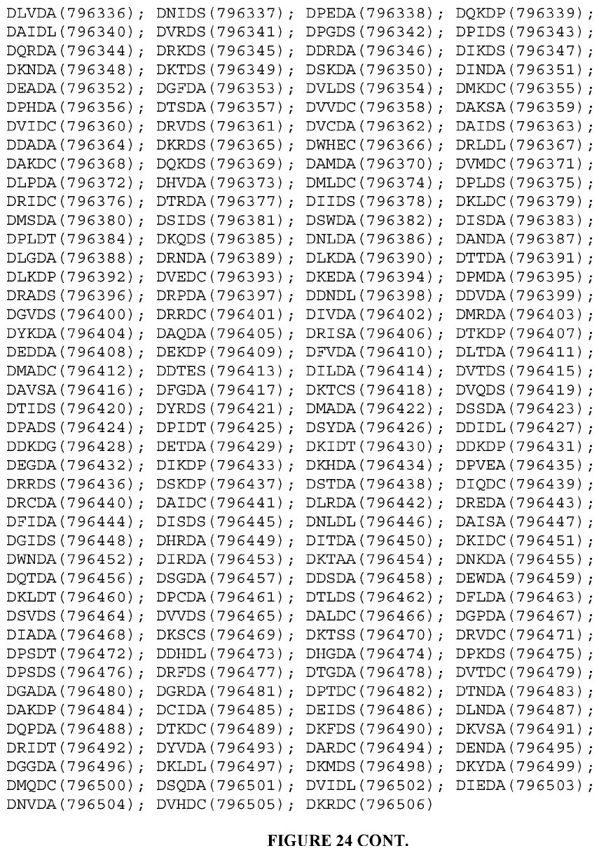

In some embodiments, the IS110 transposases belonging to the IS110 group comprise a RuvC-like DEDD catalytic domain comprising a domain motif provided in FIG. 21 with motifs in common Prosite format, where x is any amino acid and x(n) represents n number of any amino acid and x(n,m) represents n to m number of any amino acids. In some embodiments, the IS110 transposases belonging to the IS110 group comprise a RuvC-like DEDD catalytic domain comprising one or more of the RuvC-like DEDD catalytic domain motifs provided in FIGS. 22-24 with motifs in common Prosite format, where x is any amino acid and x(n) represents n number of any amino acid and x(n,m) represents n to m number of any amino acids. In some embodiments, the IS110 transposases belonging to the IS110 group comprises a RuvC-like DEDD catalytic domain wherein the “D” region of the canonical DEDD catalytic motif comprises a domain motif provided in FIG. 22 with motifs in common Prosite format, where x is any amino acid and x(n) represents n number of any amino acid and x(n,m) represents n to m number of any amino acids. In some embodiments, the IS110 transposases belonging to the IS110 group comprises a RuvC-like DEDD catalytic domain wherein the “E” region of the canonical DEDD catalytic motif comprises a domain motif provided in FIG. 23 with motifs in common Prosite format, where x is any amino acid and x(n) represents n number of any amino acid and x(n,m) represents n to m number of any amino acids. In some embodiments, the IS110 transposases belonging to the IS110 group comprises a RuvC-like DEDD catalytic domain wherein the “DD” region of the canonical DEDD catalytic motif comprises a domain motif provided in FIG. 24 with motifs in common Prosite format, where x is any amino acid and x(n) represents n number of any amino acid and x(n,m) represents n to m number of any amino acids. In some embodiments, the I110 transposases belonging to the I110 group comprises a RuvC-like DEDD catalytic domain wherein the “D” region of the canonical DEDD catalytic motif comprises a domain motif provided in FIG. 22, the “E” region of the canonical DEDD catalytic motif comprises a domain motif provided in FIG. 23, and the “DD” region of the canonical DEDD catalytic motif comprises a domain motif provided in FIG. 24 with motifs in common Prosite format, where x is any amino acid and x(n) represents n number of any amino acid and x(n,m) represents n to m number of any amino acids.

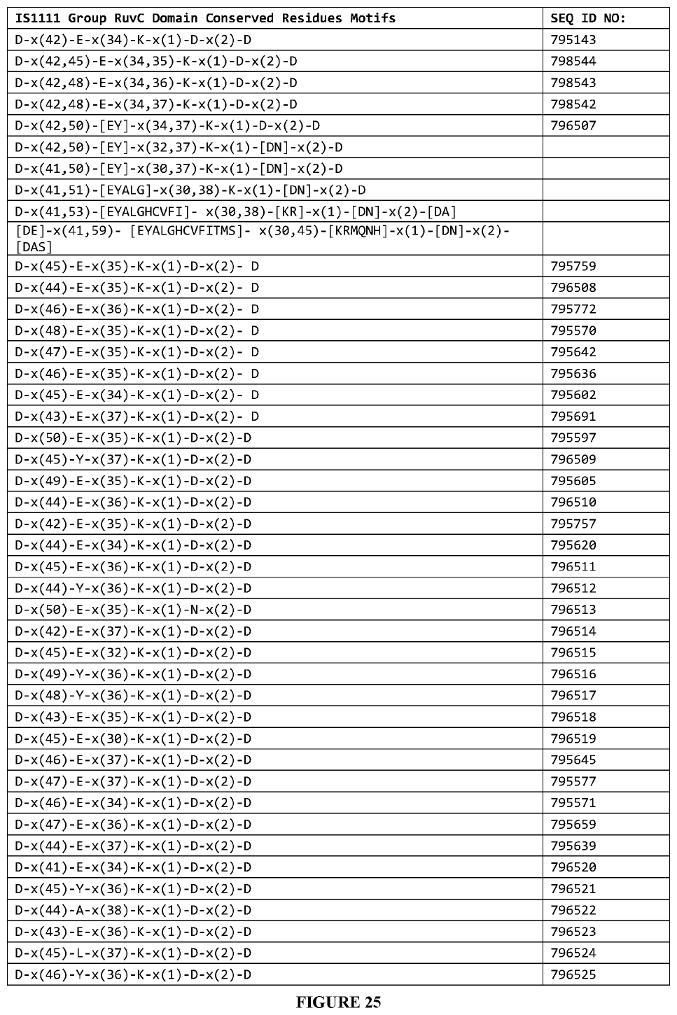

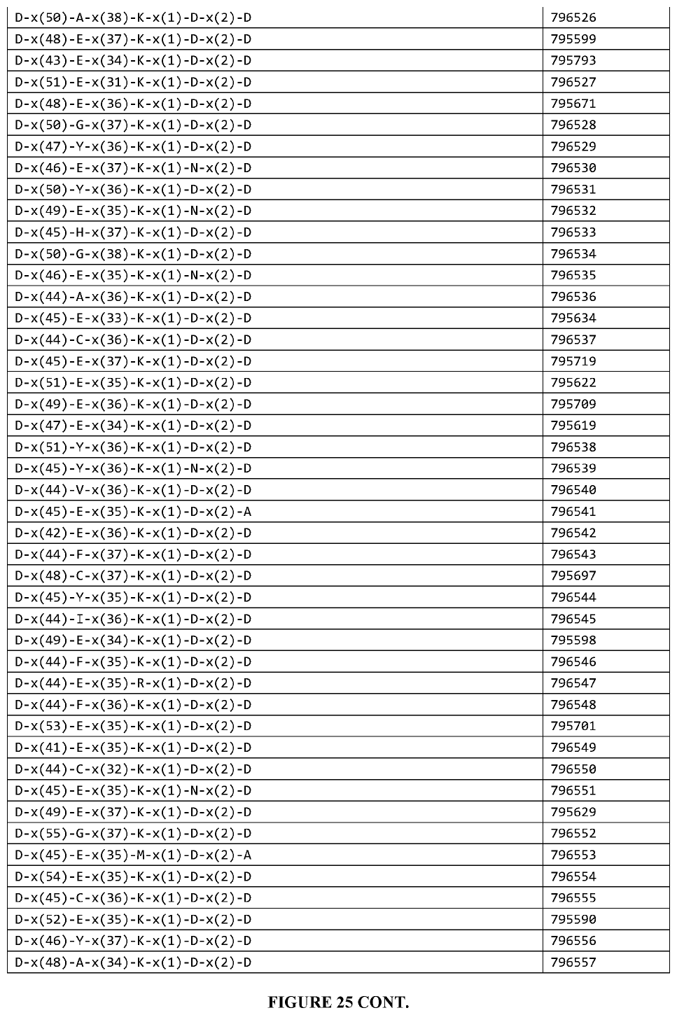

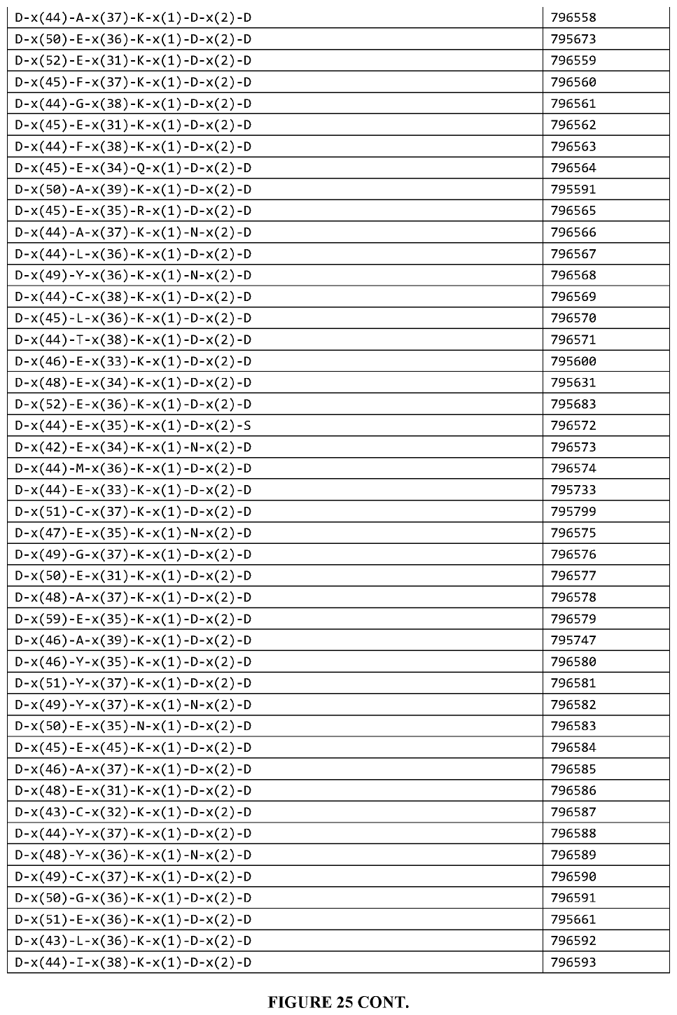

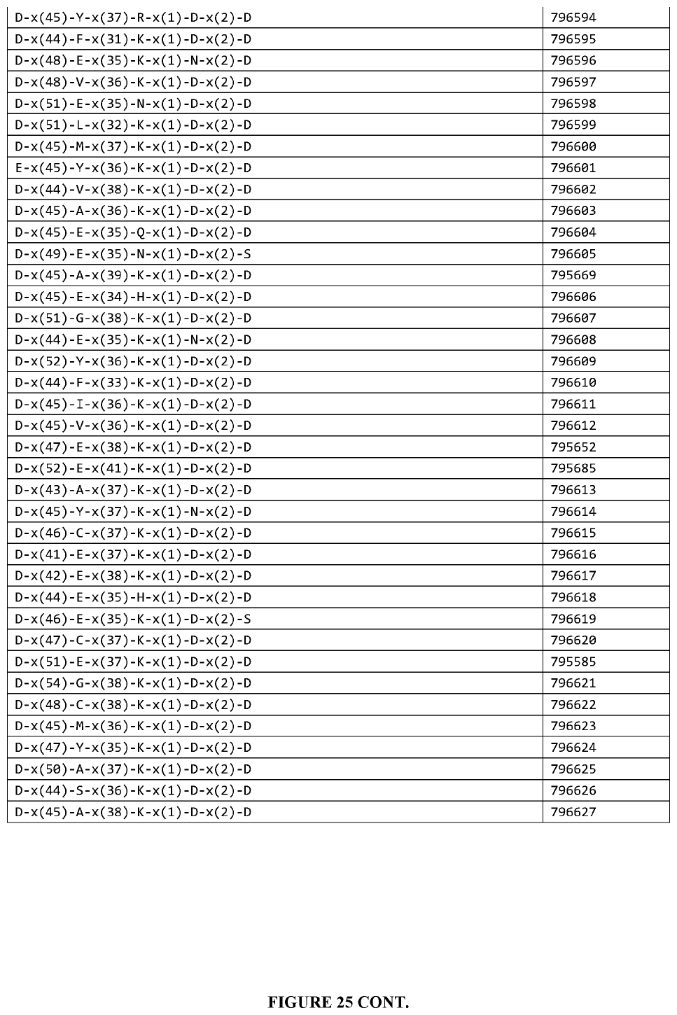

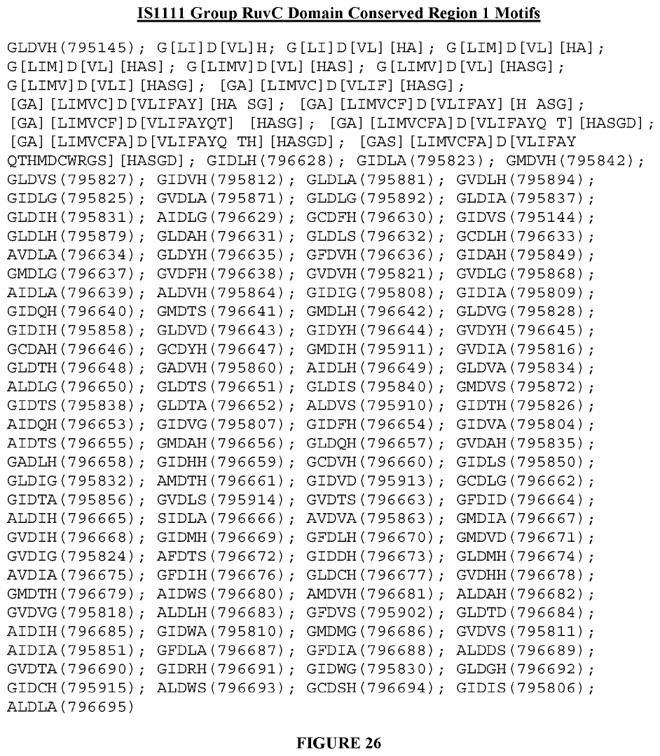

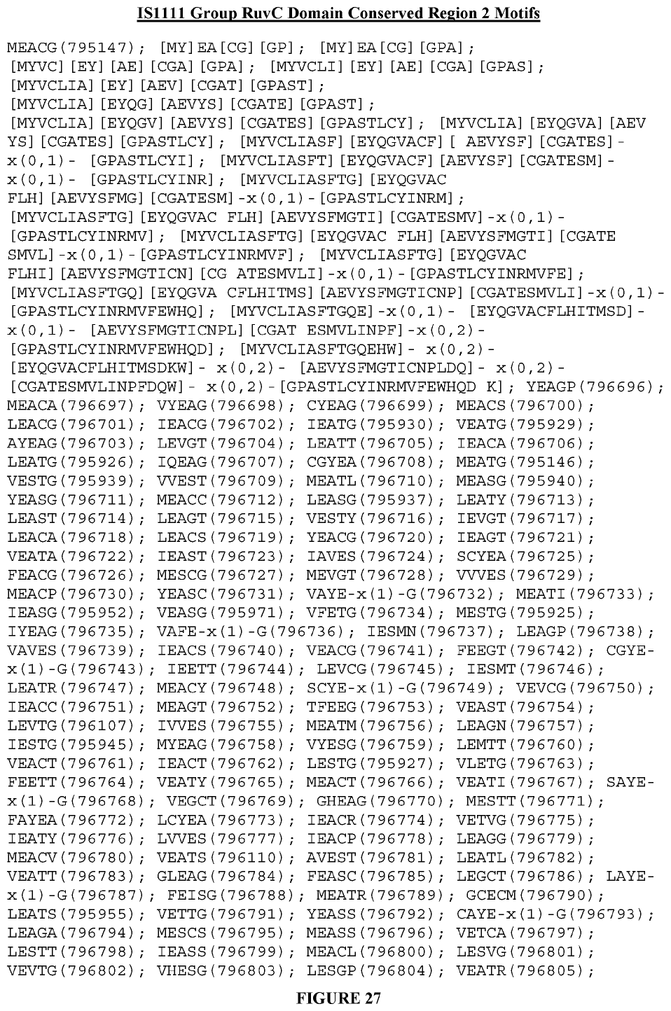

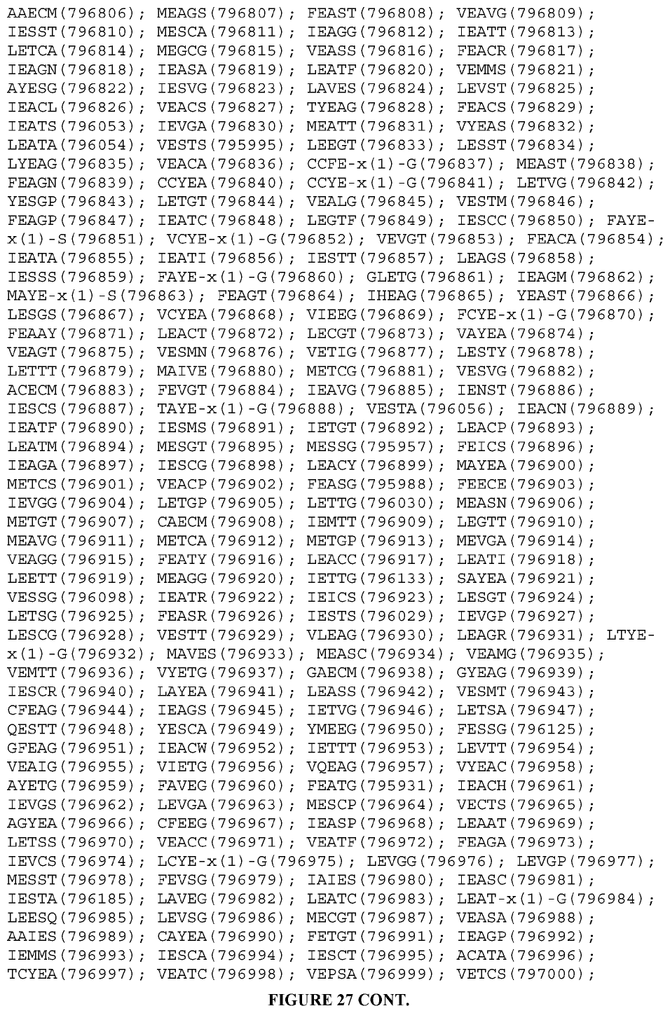

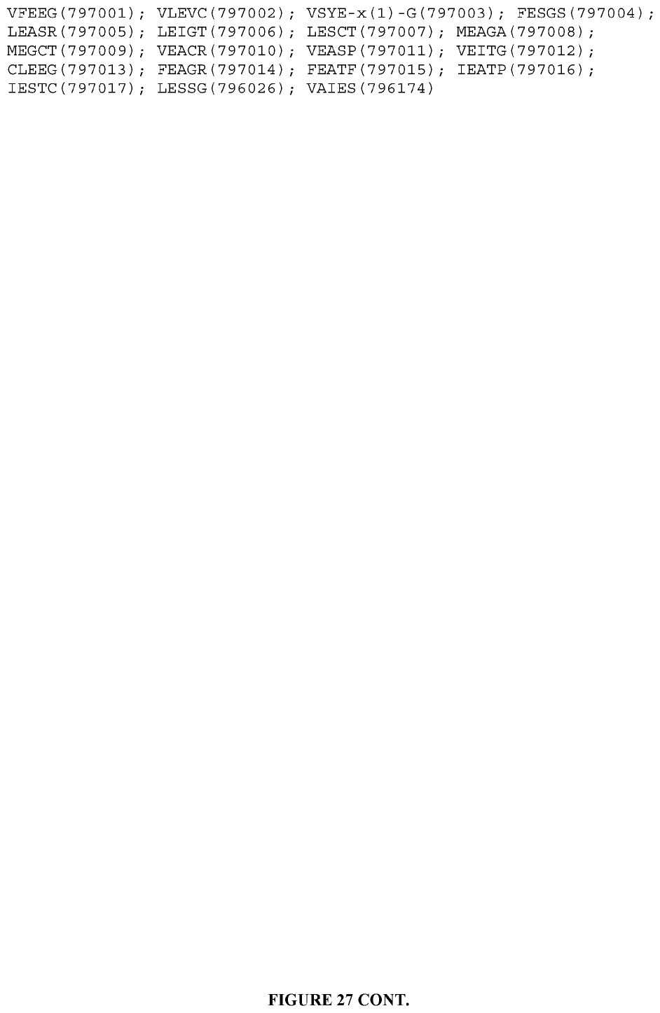

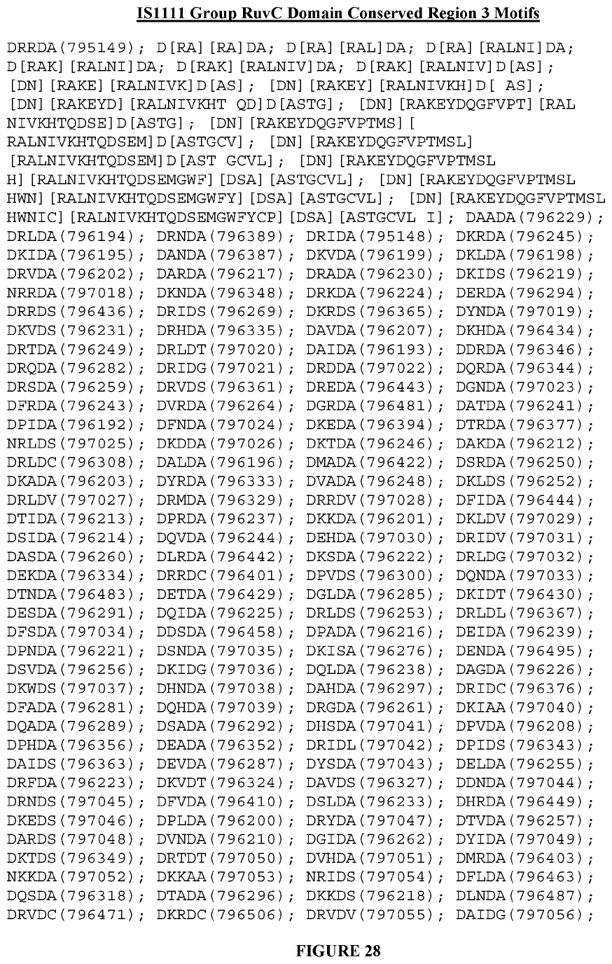

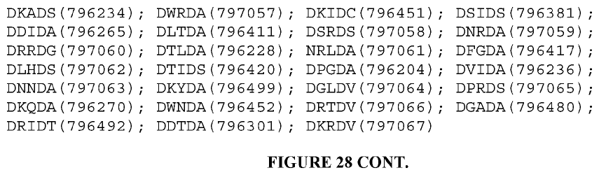

In some embodiments, the IS110 transposases belonging to the IS1111 group comprise a RuvC-like DEDD catalytic domain comprising a domain motif provided in FIG. 25 with motifs in common Prosite format, where x is any amino acid and x(n) represents n number of any amino acid and x(n,m) represents n to m number of any amino acids. In some embodiments, the IS110 transposases belonging to the IS1111 group comprise a RuvC-like DEDD catalytic domain comprising one or more of the RuvC-like DEDD catalytic domain motifs provided in FIGS. 26-28 with motifs in common Prosite format, where x is any amino acid and x(n) represents n number of any amino acid and x(n,m) represents n to m number of any amino acids. In some embodiments, the IS110 transposases belonging to the IS1111 group comprises a RuvC-like DEDD catalytic domain wherein the “D” region of the canonical DEDD catalytic motif comprises a domain motif provided in FIG. 26 with motifs in common Prosite format, where x is any amino acid and x(n) represents n number of any amino acid and x(n,m) represents n to m number of any amino acids. In some embodiments, the IS110 transposases belonging to the IS1111 group comprises a RuvC-like DEDD catalytic domain wherein the “E” region of the canonical DEDD catalytic motif comprises a domain motif provided in FIG. 27 with motifs in common Prosite format, where x is any amino acid and x(n) represents n number of any amino acid and x(n,m) represents n to m number of any amino acids. In some embodiments, the IS110 transposases belonging to the IS1111 group comprises a RuvC-like DEDD catalytic domain wherein the “DD” region of the canonical DEDD catalytic motif comprises a domain motif provided in FIG. 28 with motifs in common Prosite format, where x is any amino acid and x(n) represents n number of any amino acid and x(n,m) represents n to m number of any amino acids. In some embodiments, the IS110 transposases belonging to the IS1111 group comprises a RuvC-like DEDD catalytic domain wherein the “D” region of the canonical DEDD catalytic motif comprises a domain motif provided in FIG. 26, the “E” region of the canonical DEDD catalytic motif comprises a domain motif provided in FIG. 27, and the “DD” region of the canonical DEDD catalytic motif comprises a domain motif provided in FIG. 28 with motifs in common Prosite format, where x is any amino acid and x(n) represents n number of any amino acid and x(n,m) represents n to m number of any amino acids.

The present invention contemplates domain swapping in order to generate IS110 transposase chimeras that have advantageous functions. In some embodiments, exchanging of a RuvC-like DEDD catalytic domain (e.g., any RuvC-like DEDD catalytic domain provided in FIG. 16 (SEQ ID NOS: 10176-10523) or SEQ ID NOS: 10524-20350 or 40357-516430) with a different RuvC-like DEDD catalytic domain (e.g., any other RuvC-like DEDD catalytic domain provided in FIG. 16 (SEQ ID NOS: 10176-10523) or SEQ ID NOS: 10524-20350 or 40357-516430) resulting in advantageous properties is also envisioned as described in Farruggio et al., 2014. For example, exchanging one RuvC-like DEDD catalytic domain with another RuvC-like DEDD catalytic domain may allow for higher affinity for a bridgeRNA or increased transposition efficiency.

### C.2. Transposase Domain

The IS110 family transposases described herein comprise a transposase domain.

In some embodiments, the transposase domain can be identified using statistical models that annotate protein domains, such as Pfam profile hidden markov models (pHMMs). In IS110 family transposases, the transposase domain is often recognized by the Pfam profile hidden markov model (pHEMM) PF02371, short name Transposase_20.

In some embodiments, the IS110 family transposase comprises a transposase domain comprising an amino acid sequence that is 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the transposase domain sequences provided in FIG. 16 (SEQ ID NOS: 10176-10523) or SEQ ID NOS: 10524-20350 or 40357-516430. In some embodiments the transposase domain sequence provided is the transposase domain of “protein_IS621” of FIG. 16 (SEQ ID NO:10176).

In some embodiments, the IS110 family transposase comprises a transposase domain that forms a similar tertiary structure to the transposase domain of IS621. In some embodiments, the tertiary structure is determined using AlphaFold or similar protein structure prediction software. In some embodiments, a particular amino acid sequence is considered to form a similar tertiary structure to the transposase domain of IS621 if the template modeling score (TM-score) for the transposase domain is 0.5 or higher. In some embodiments, a particular amino acid sequence is considered to form a similar tertiary structure to the transposase domain of IS621 if the template modeling score (TM-score) is 0.5 or higher, 0.6 or higher, 0.7 or higher, 0.8 or higher, or 0.9 or higher.

In some embodiments, a particular amino acid sequence is considered to form a similar tertiary structure to the transposase domain of IS621 based on distances between the alpha carbon of conserved residues in the transposase domain. FIG. 16 (SEQ ID NOS: 10176-10523) provides in bold typeface amino acids up to 5 amino acids that are highly conserved in the transposase domain. SEQ ID NOS: 10524-20350 or 40357-516430 provide as features P1-P5 of the sequence listing up to 5 amino acids that are highly conserved in the transposase domain. Conserved amino acids in a particular amino acid sequence are identified by primary amino acid sequence alignment. In some embodiments, a particular amino acid sequence is considered to form a similar tertiary structure to the transposase domain of IS621 if an average distance (“D1”) between the alpha carbon of a first conserved residues and the alpha carbon of a second conserved residue and between the alpha carbon of a first conserved residue and the alpha carbon of a fifth conserved residue of the amino acid sequence is less than 25 angstroms (Å), wherein the conserved residues of the amino acid sequence is per alignment of the primary amino acid sequence with one or more transposase domains, such as IS621. In IS621 the first conserved residue is G203, the second conserved residue is G233, and the fifth conserved residue is G255. In some embodiments, D1 is between 15 and 25 angstroms (Å). In some embodiments, D1 is between 17 and 23 angstroms (Å). In some embodiments, a particular amino acid sequence is considered to form a similar tertiary structure to the transposase domain of IS621 if an average distance (“D2”) between the alpha carbon of a second conserved residue and the alpha carbon of a third conserved residue, between the alpha carbon of a second conserved residue and the alpha carbon of a fourth conserved residue, between the alpha carbon of a fifth conserved residue and the alpha carbon of a third conserved residue, and between the alpha carbon of a fifth conserved residue and the alpha carbon of a fourth conserved residue, is less than 25 angstroms (Å), wherein the conserved residues of the amino acid sequence is per alignment of the primary amino acid sequence with one or more transposase domains, such as IS621. In IS621 the second conserved residue is G233, the third conserved residue is S241, the fourth conserved residue is G242, and the fifth conserved residue is G255. In some embodiments, D2 is between 20 and 25 angstroms (Å). In some embodiments, D2 is between 22 and 24 angstroms (Å). In some embodiments, a particular amino acid sequence is considered to form a similar tertiary structure to the transposase of IS621 if a distance (“D3”) between the alpha carbon of a second conserved residue and the alpha carbon of a fifth conserved residue is less than 15 angstroms (Å), wherein the conserved residues of the amino acid sequence is per alignment of the primary amino acid sequence with one or more transposase domains, such as IS621. In IS621 the second conserved residue is G233 and the fifth conserved residue is G255. In some embodiments, D3 is between 5 and 15 angstroms (Å). In some embodiments, D3 is between 7 and 12 angstroms (Å). In some embodiments, a particular amino acid sequence is considered to form a similar tertiary structure to the transposase domain of IS621 if D1 is less than 25 angstroms (Å), D2 is less than 25 angstroms (Å), and D3 is less than 15 angstroms (Å). In some embodiments, a particular amino acid sequence is considered to form a similar tertiary structure to the transposase domain of IS621 if D1 is between 15 and 25 angstroms (Å), D2 is between 20 and 25 angstroms (Å), and D3 is between 5 and 15 angstroms (Å). In some embodiments, a particular amino acid sequence is considered to form a similar tertiary structure to the transposase domain of IS621 if D1 is between 17 and 23 angstroms (Å), D2 is between 22 and 24 angstroms (Å), and D3 is between 7 and 12 angstroms (Å).

In some embodiments, the IS110 family transposase comprises a transposase domain comprising an amino acid sequence that is 15% identical or more, 16% identical or more, 17% identical or more, 18% identical or more, 19% identical or more, 20% identical or more, 21% identical or more, 22% identical or more, 23% identical or more, 24% identical or more, 25% identical or more, 26% identical or more, 27% identical or more, 28% identical or more, 29% identical or more, 30% identical or more, 31% identical or more, 32% identical or more, 33% identical or more, 34% identical or more, 35% identical or more, 36% identical or more, 37% identical or more, 38% identical or more, 39% identical or more, 40% identical or more, 41% identical or more, 42% identical or more, 43% identical or more, 44% identical or more, 45% identical or more, 46% identical or more, 47% identical or more, 48% identical or more, 49% identical or more, 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the transposase domain sequences provided in FIG. 16 (SEQ ID NOS: 10176-10523) or SEQ ID NOS: 10524-20350 or 40357-516430 and the amino acid sequence forms a similar tertiary structure to the transposase domain of IS621. In some embodiments, the tertiary structure is determined using AlphaFold or similar protein structure prediction software. In some embodiments, a particular amino acid sequence is considered to form a similar tertiary structure to the transposase domain of IS621 if the template modeling score (TM-score) for the transposase domain is 0.5 or higher. In some embodiments, a particular amino acid sequence is considered to form a similar tertiary structure to the transposase domain of IS621 if the template modeling score (TM-score) is 0.5 or higher, 0.6 or higher, 0.7 or higher, 0.8 or higher, or 0.9 or higher. In some embodiments the transposase domain sequence provided is the transposase domain of “protein_IS621” of FIG. 16 (SEQ ID NO:10176).

In some embodiments, the IS110 family transposase comprises a RuvC-like DEDD catalytic domain described in the preceding paragraphs (see Section C.1.) and further comprises a transposase domain comprising an amino acid sequence that is 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the transposase domain sequences provided in FIG. 16 (SEQ ID NOS: 10176-10523) or SEQ ID NOS: 10524-20350 or 40357-516430. In some embodiments, the amino acid sequence forms a similar tertiary structure to the transposase domain of IS621. In some embodiments, the tertiary structure is determined using AlphaFold or similar protein structure prediction software. In some embodiments, a particular amino acid sequence is considered to form a similar tertiary structure to the transposase domain of IS621 if the template modeling score (TM-score) for the transposase domain is 0.5 or higher. In some embodiments, a particular amino acid sequence is considered to form a similar tertiary structure to the transposase domain of IS621 if the template modeling score (TM-score) is 0.5 or higher, 0.6 or higher, 0.7 or higher, 0.8 or higher, or 0.9 or higher. In some embodiments the transposase domain sequence provided is the transposase domain of “protein_IS621” of FIG. 16 (SEQ ID NO:10176).

In some embodiments, the IS110 family transposase comprises a RuvC-like DEDD catalytic domain described in the preceding paragraphs (see Section C.1.) and further comprises a transposase domain that the amino acid sequence forms a similar tertiary structure to the transposase domain of IS621. In some embodiments, the tertiary structure is determined using AlphaFold or similar protein structure prediction software. In some embodiments, a particular amino acid sequence is considered to form a similar tertiary structure to the transposase domain of IS621 if the template modeling score (TM-score) for the transposase domain is 0.5 or higher. In some embodiments, a particular amino acid sequence is considered to form a similar tertiary structure to the transposase domain of IS621 if the template modeling score (TM-score) is 0.5 or higher, 0.6 or higher, 0.7 or higher, 0.8 or higher, or 0.9 or higher. In some embodiments the transposase domain sequence provided is the transposase domain of “protein_IS621” of FIG. 16 (SEQ ID NO:10176). In some embodiments, the amino acid sequence forms a similar tertiary structure to the RuvC-like DEDD catalytic domain of IS621. In some embodiments, the tertiary structure is determined using AlphaFold or similar protein structure prediction software. In some embodiments, a particular amino acid sequence is considered to form a similar tertiary structure to the RuvC-like DEDD catalytic domain of IS621 if the template modeling score (TM-score) for the RuvC-like DEDD catalytic domain is 0.5 or higher. In some embodiments, a particular amino acid sequence is considered to form a similar tertiary structure to the RuvC-like DEDD catalytic domain of IS621 if the template modeling score (TM-score) is 0.5 or higher, 0.6 or higher, 0.7 or higher, 0.8 or higher, or 0.9 or higher. In some embodiments the RuvC-like DEDD catalytic domain sequence provided is the RuvC-like DEDD catalytic domain of “protein_IS621” of FIG. 16 (SEQ ID NO:10176). In some embodiments, the transposase domain comprises an amino acid sequence that is 15% identical or more, 16% identical or more, 17% identical or more, 18% identical or more, 19% identical or more, 20% identical or more, 21% identical or more, 22% identical or more, 23% identical or more, 24% identical or more, 25% identical or more, 26% identical or more, 27% identical or more, 28% identical or more, 29% identical or more, 30% identical or more, 31% identical or more, 32% identical or more, 33% identical or more, 34% identical or more, 35% identical or more, 36% identical or more, 37% identical or more, 38% identical or more, 39% identical or more, 40% identical or more, 41% identical or more, 42% identical or more, 43% identical or more, 44% identical or more, 45% identical or more, 46% identical or more, 47% identical or more, 48% identical or more, 49% identical or more, 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the transposase domain sequences provided in FIG. 16 (SEQ ID NOS: 10176-10523) or SEQ ID NOS: 10524-20350 or 40357-516430.

In some embodiments, the IS110 family transposase comprises a RuvC-like DEDD catalytic domain comprising an amino acid sequence that is 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the RuvC-like DEDD catalytic domain sequences provided in FIG. 16 (SEQ ID NOS: 10176-10523) or SEQ ID NOS: 10524-20350 or 40357-516430 and further comprises a transposase domain comprising an amino acid sequence that is 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the transposase domain sequences provided in FIG. 16 (SEQ ID NOS: 10176-10523) or SEQ ID NOS: 10524-20350 or 40357-516430. In some embodiments, the amino acid sequence forms a similar tertiary structure to the RuvC-like DEDD catalytic domain of IS621. In some embodiments, the tertiary structure is determined using AlphaFold or similar protein structure prediction software. In some embodiments, a particular amino acid sequence is considered to form a similar tertiary structure to the RuvC-like DEDD catalytic domain of IS621 if the template modeling score (TM-score) for the RuvC-like DEDD catalytic domain is 0.5 or higher. In some embodiments, a particular amino acid sequence is considered to form a similar tertiary structure to the RuvC-like DEDD catalytic domain of IS621 if the template modeling score (TM-score) is 0.5 or higher, 0.6 or higher, 0.7 or higher, 0.8 or higher, or 0.9 or higher. In some embodiments the RuvC-like DEDD catalytic domain sequence provided is the RuvC-like DEDD catalytic domain of “protein_IS621” of FIG. 16 (SEQ ID NO:10176). In some embodiments, the amino acid sequence forms a similar tertiary structure to the transposase domain of IS621. In some embodiments, the tertiary structure is determined using AlphaFold or similar protein structure prediction software. In some embodiments, a particular amino acid sequence is considered to form a similar tertiary structure to the transposase domain of IS621 if the template modeling score (TM-score) for the transposase domain is 0.5 or higher. In some embodiments, a particular amino acid sequence is considered to form a similar tertiary structure to the transposase domain of IS621 if the template modeling score (TM-score) is 0.5 or higher, 0.6 or higher, 0.7 or higher, 0.8 or higher, or 0.9 or higher. In some embodiments the transposase domain sequence provided is the transposase domain of “protein_IS621” of FIG. 16 (SEQ ID NO:10176).

In some embodiments, the IS110 family transposase comprises a transposase domain comprising a motif G-x(28)-G-x(7)-SG-x(10)-G (SEQ ID NO: 795150), G-x(29)-G-x(7)-SG-x(11)-G (SEQ ID NO: 795151), IPGIG (SEQ ID NO: 795152), IPGVG (SEQ ID NO: 795153), AGLAP (SEQ ID NO: 795154), LGLVP (SEQ ID NO: 795155), [GSRAVECPYQNHFDKIT]-x(26,39)-[GKARCTNSEQHMD]-x(7,8)-[STRP][GASDNQCVER]-x(8,18)-[GACVYTSKRFNHIQDE], [GASFCRTNQKYDEV]-x(25,31)-[GRALKMSQCED]-x(7)-[STRAP][GNADSVCHER]-x(10,17)-[GSVRCAYKHQTFINED], [IVLAMFQTERCHSKYPWNG][PKDTFYERVSHQANGILCMW][GRASTCEPKVYQNH FDI]-x(0,1)-[IVAFLMCSWTYGKHNPRE]-x(0,2)-[GSDANKQERTMHCP], [IVLHAMFQTCYEPRSGW][PRKESADYTHVNGQICWLMF][GSFAQCYTRKNWLDEV H][VIFLCMYATPSGW]-x(0,2)-[GADSQNKRTEPWHC], [AVLNCSTIGMFYWDHEPQR][GRTKNACSEHQMD][LVTIMAYSFCQRWKNPGH][A CDTSVNERHFWIYQGKLMP][PVLAWISTCGNMR], or [LAVIFTCSWMGYPRQNKE][GRLAMCKEQDS][LIVTCMKSAFRPQG][VTAICRNDGS QMHLEPYKF][PGKASVIDRLTENQCW] with motifs in common Prosite format, where x is any amino acid and x(n) represents n number of any amino acid and x(n,m) represents n to m number of any amino acids. In some embodiments, the transposase domain comprises a motif G-x(28)-G-x(7)-SG-x(10)-G (SEQ ID NO: 795150), G-x(29)-G-x(7)-SG-x(11)-G (SEQ ID NO: 795151), IPGIG (SEQ ID NO: 795152), IPGVG (SEQ ID NO: 795153), AGLAP (SEQ ID NO: 795154), or LGLVP (SEQ ID NO: 795155). In some embodiments, the amino acid sequence forms a similar tertiary structure to the transposase domain of IS621. In some embodiments, the tertiary structure is determined using AlphaFold or similar protein structure prediction software. In some embodiments, a particular amino acid sequence is considered to form a similar tertiary structure to the transposase domain of IS621 if the template modeling score (TM-score) for the transposase domain is 0.5 or higher. In some embodiments, a particular amino acid sequence is considered to form a similar tertiary structure to the transposase domain of IS621 if the template modeling score (TM-score) is 0.5 or higher, 0.6 or higher, 0.7 or higher, 0.8 or higher, or 0.9 or higher.

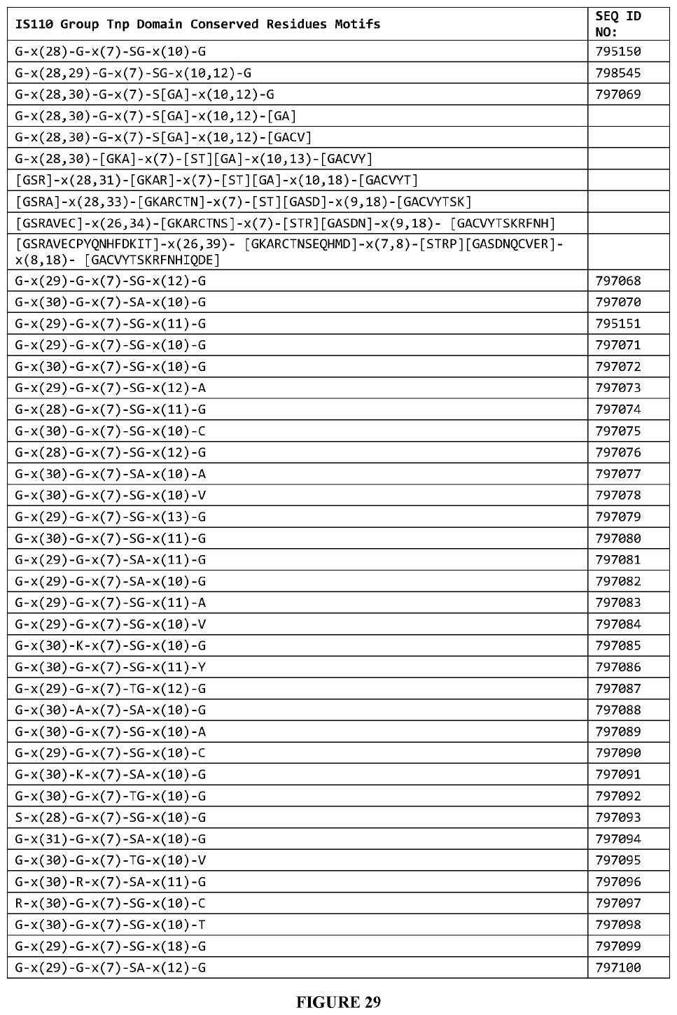

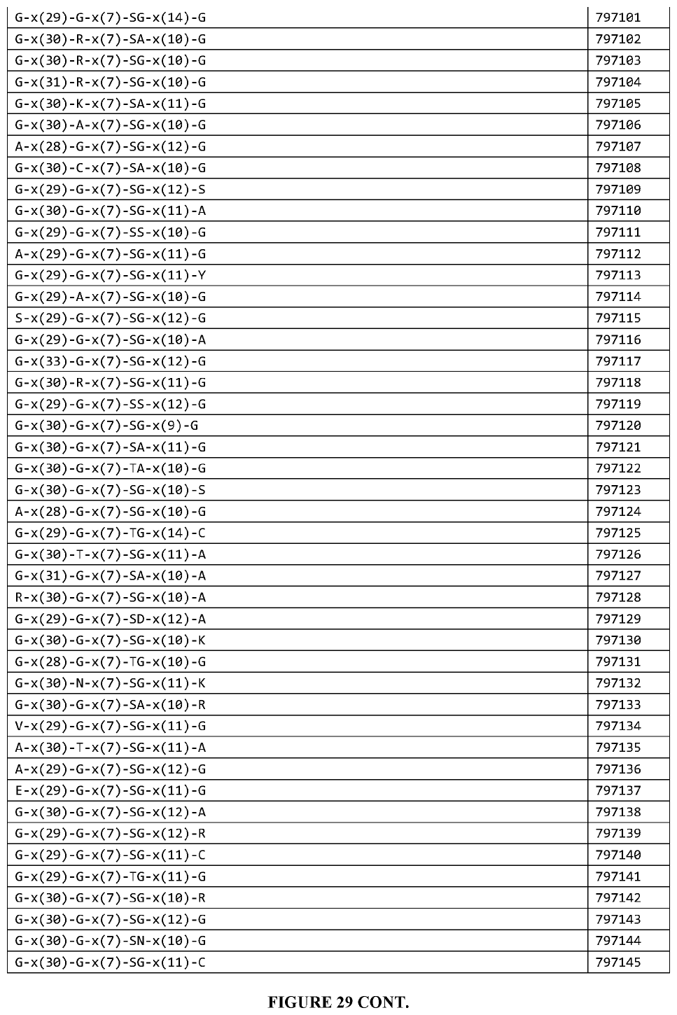

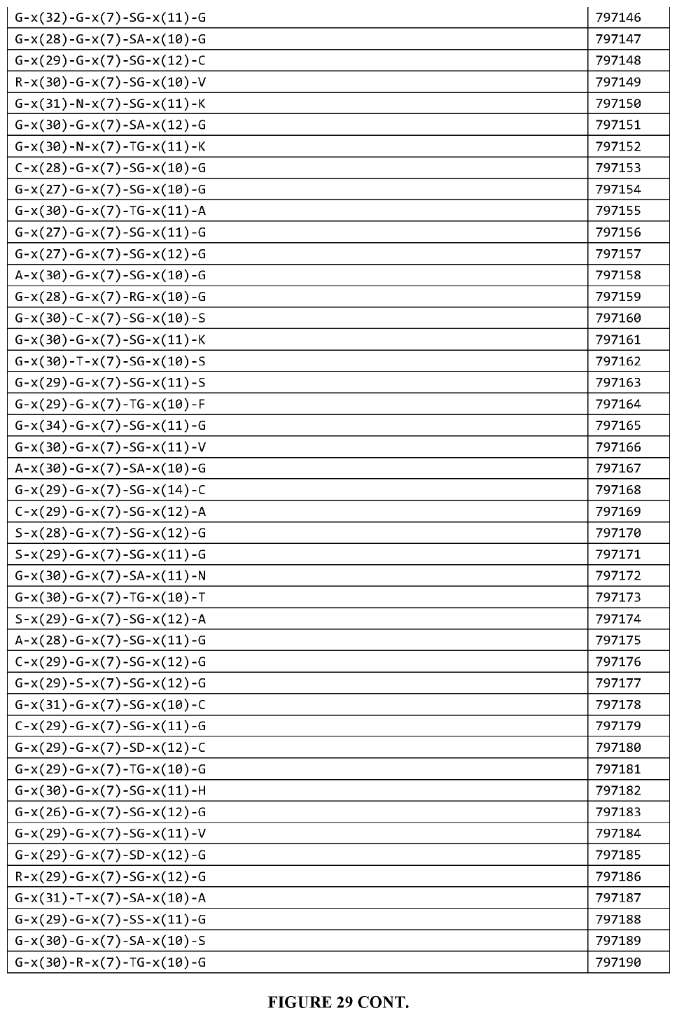

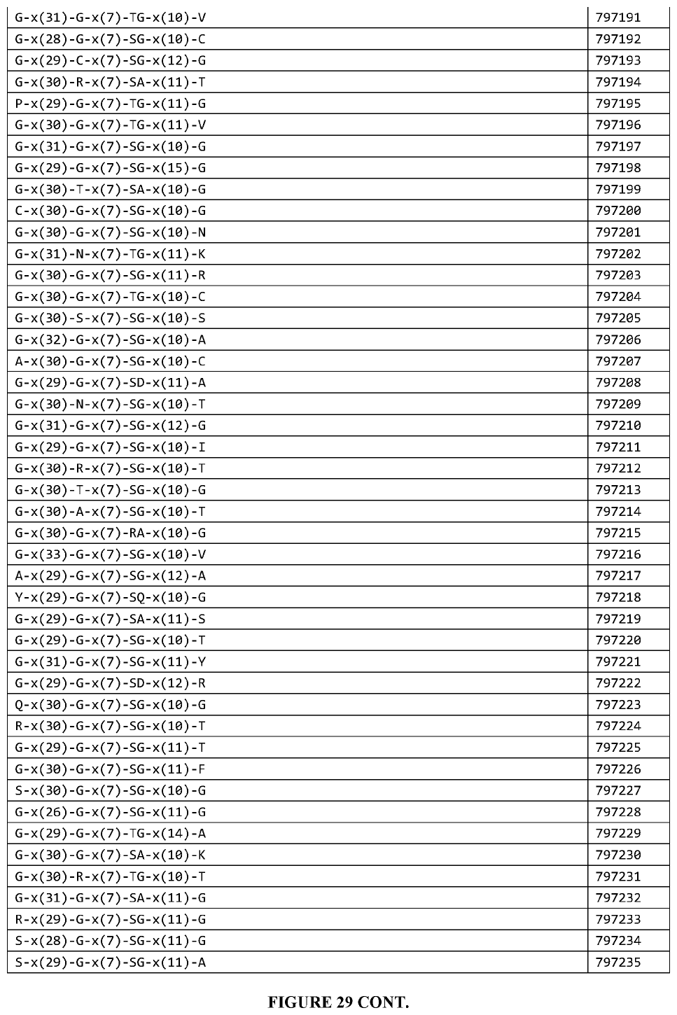

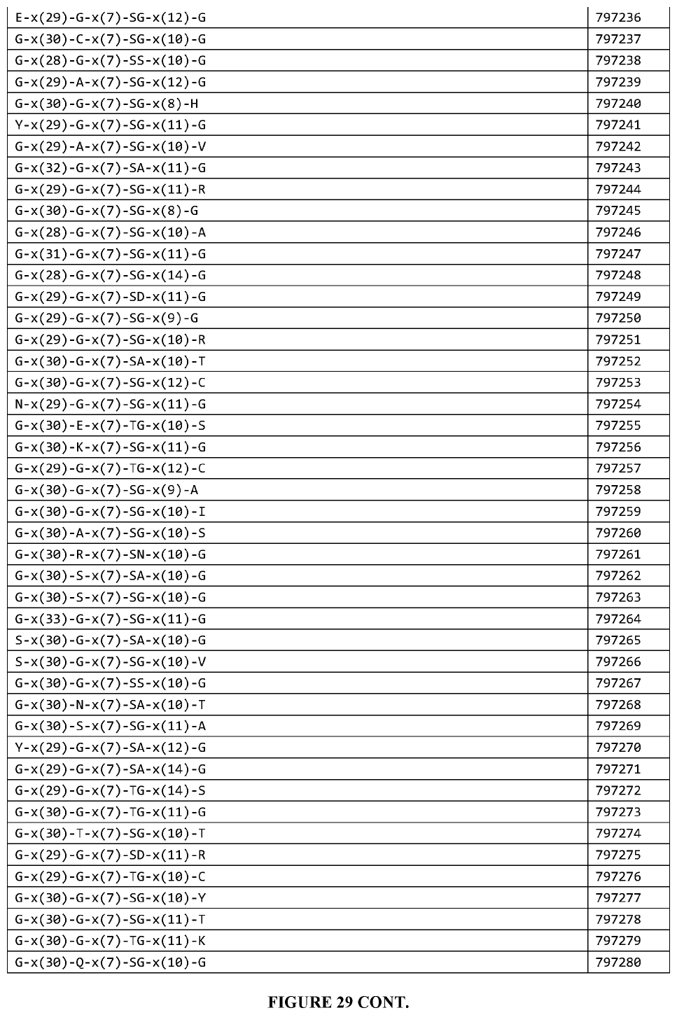

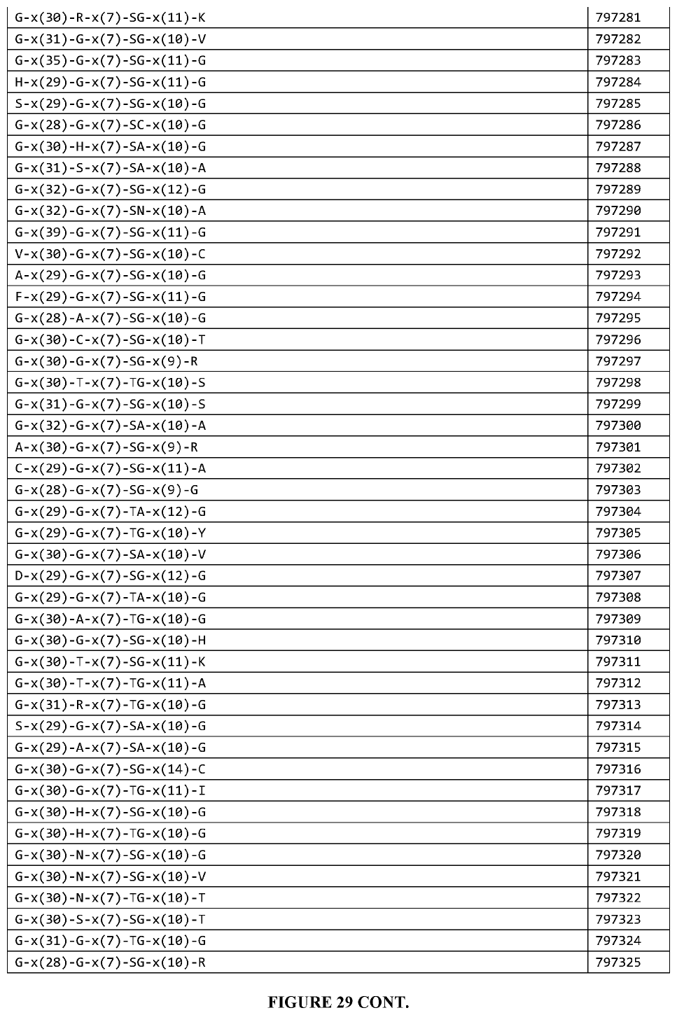

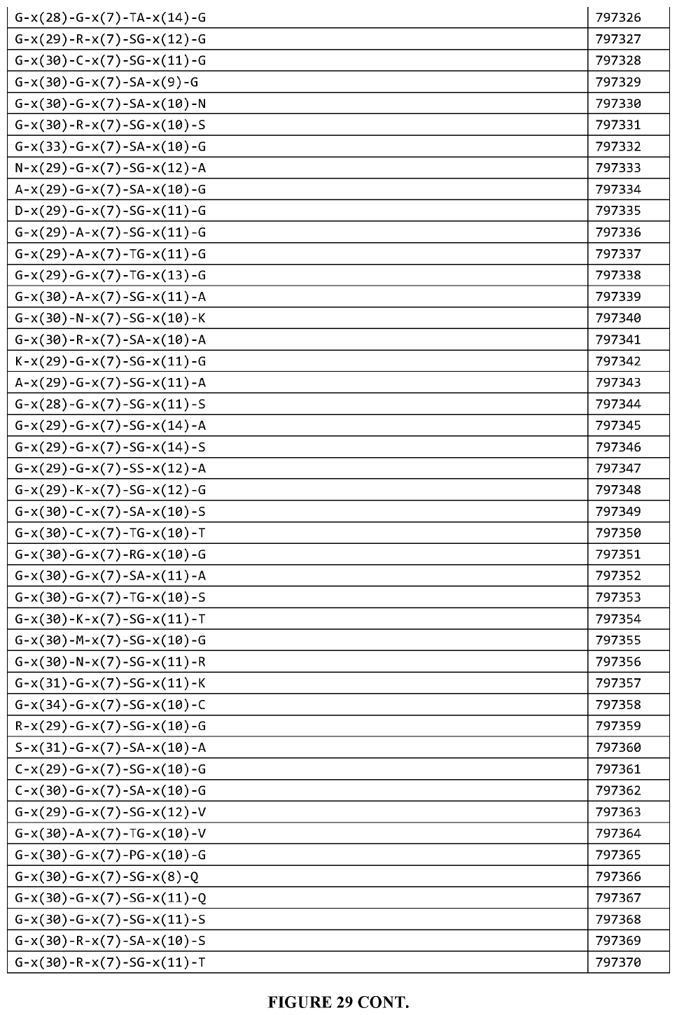

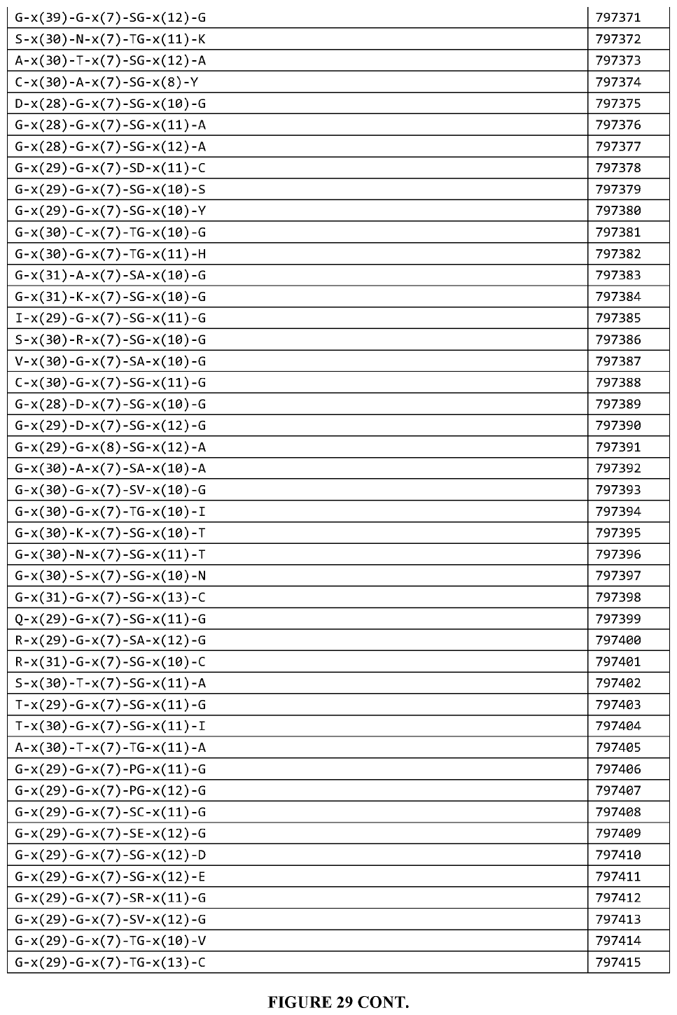

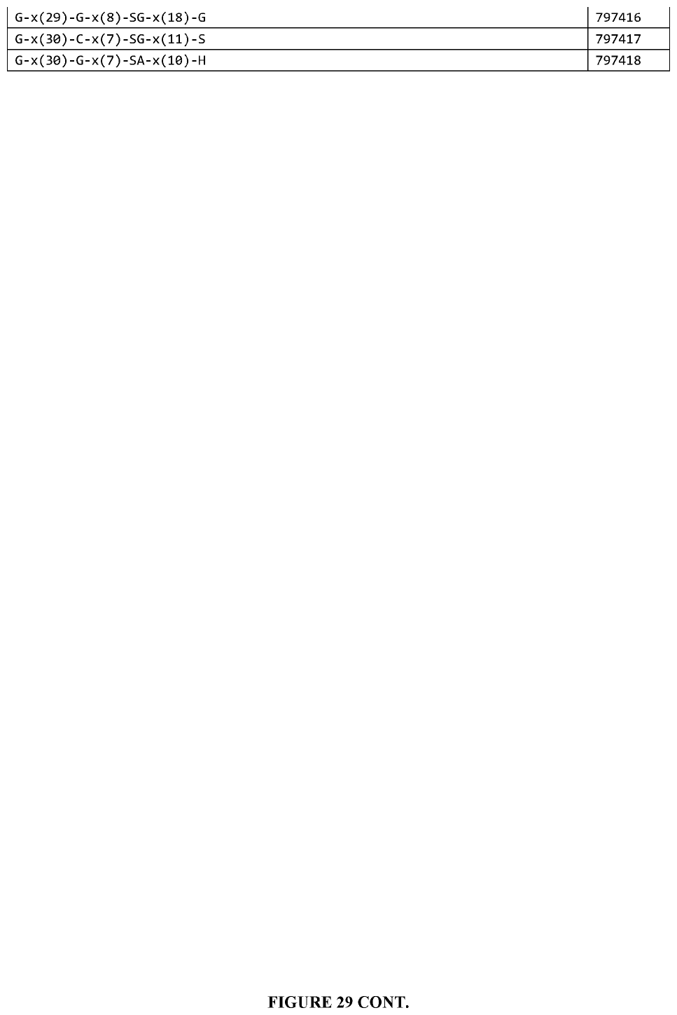

In some embodiments, the IS110 family transposase comprises a transposase domain comprising an amino acid sequence that is 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to a transposase domain sequence of FIG. 16 (SEQ ID NOS: 10176-10523) or SEQ ID NOS: 10524-20350 or 40357-516430 and comprising any of the motifs in the preceding paragraph or in FIGS. 29-34. In some embodiments, the amino acid sequence forms a similar tertiary structure to the transposase domain of IS621. In some embodiments, the tertiary structure is determined using AlphaFold or similar protein structure prediction software. In some embodiments, a particular amino acid sequence is considered to form a similar tertiary structure to the transposase domain of IS621 if the template modeling score (TM-score) for the transposase domain is 0.5 or higher. In some embodiments, a particular amino acid sequence is considered to form a similar tertiary structure to the transposase domain of IS621 if the template modeling score (TM-score) is 0.5 or higher, 0.6 or higher, 0.7 or higher, 0.8 or higher, or 0.9 or higher.

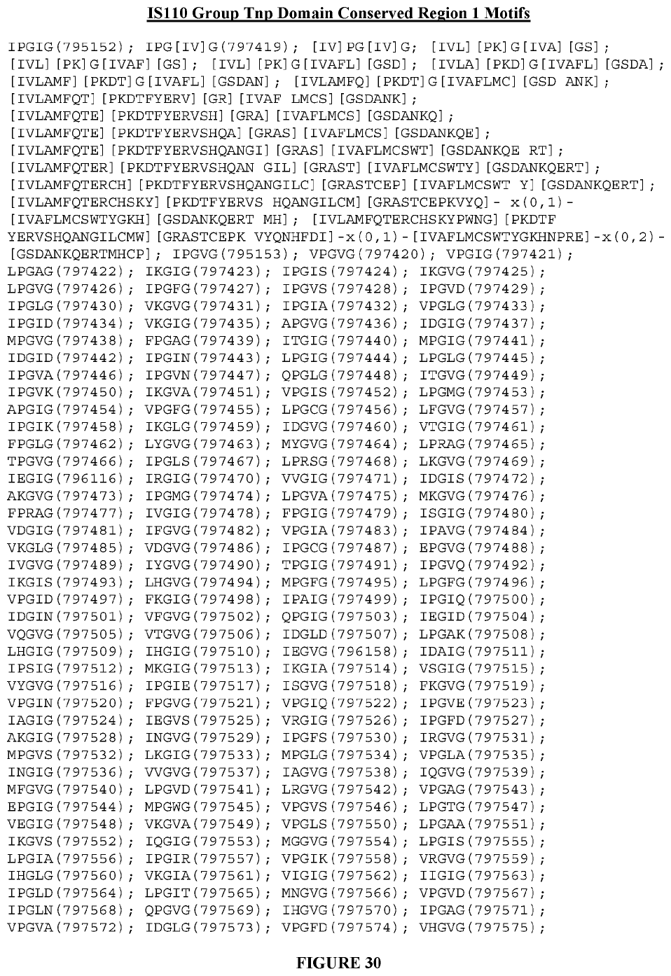

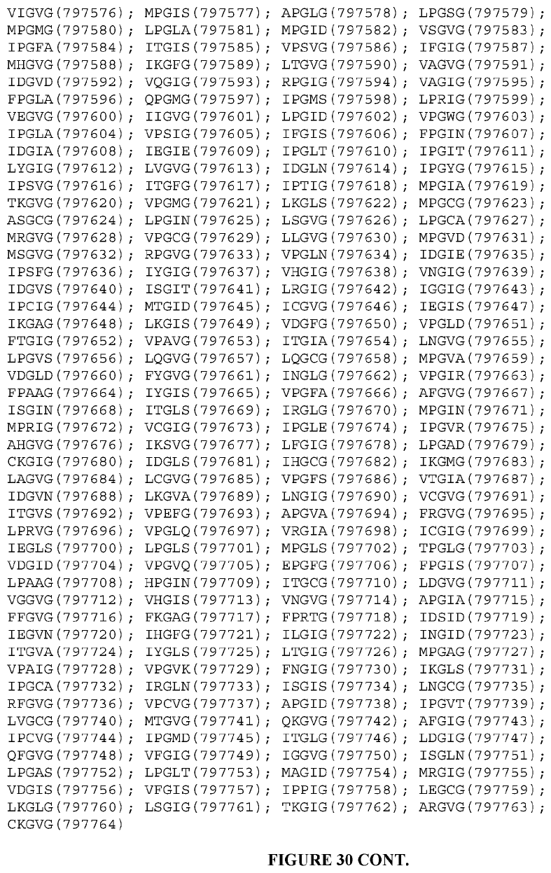

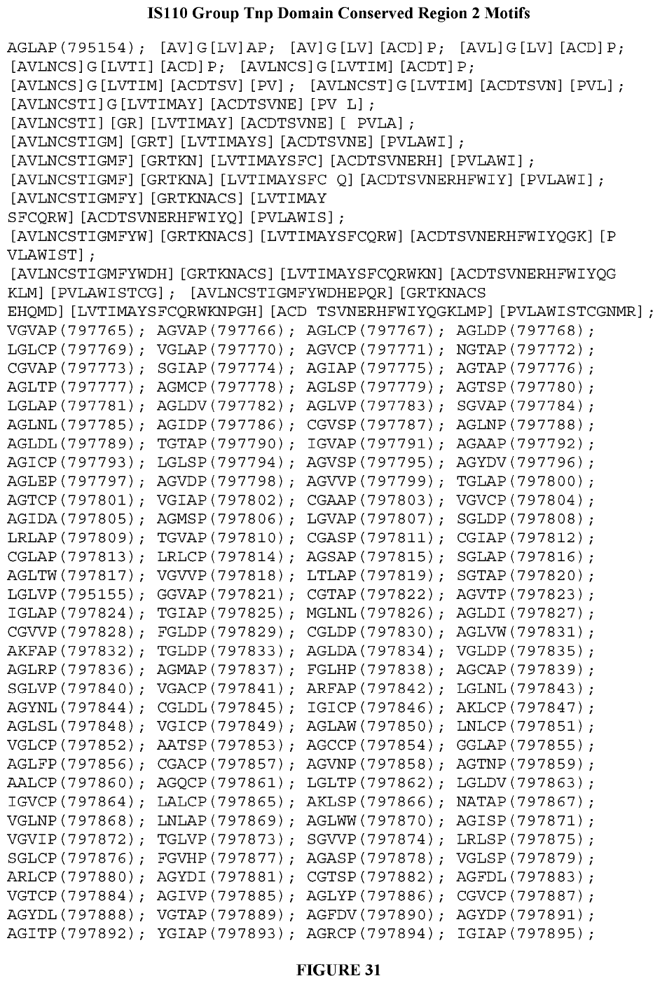

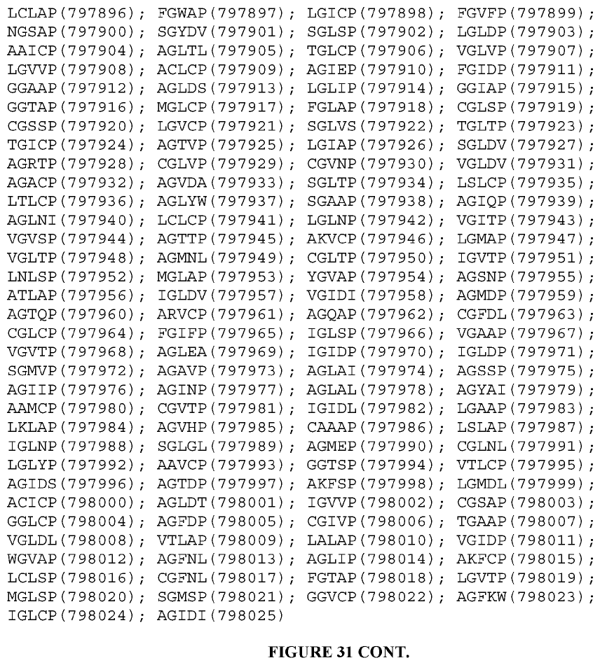

In some embodiments, the IS110 transposases belonging to the IS110 group comprise a transposase domain comprising a domain motifs provided in FIG. 29 with motifs in common Prosite format, where x is any amino acid and x(n) represents n number of any amino acid and x(n,m) represents n to m number of any amino acids. In some embodiments, the IS110 transposases belonging to the IS110 group comprise a transposase domain comprising one or more of the transposase domain motifs provided in FIGS. 30-31 with motifs in common Prosite format, where x is any amino acid and x(n) represents n number of any amino acid and x(n,m) represents n to m number of any amino acids.

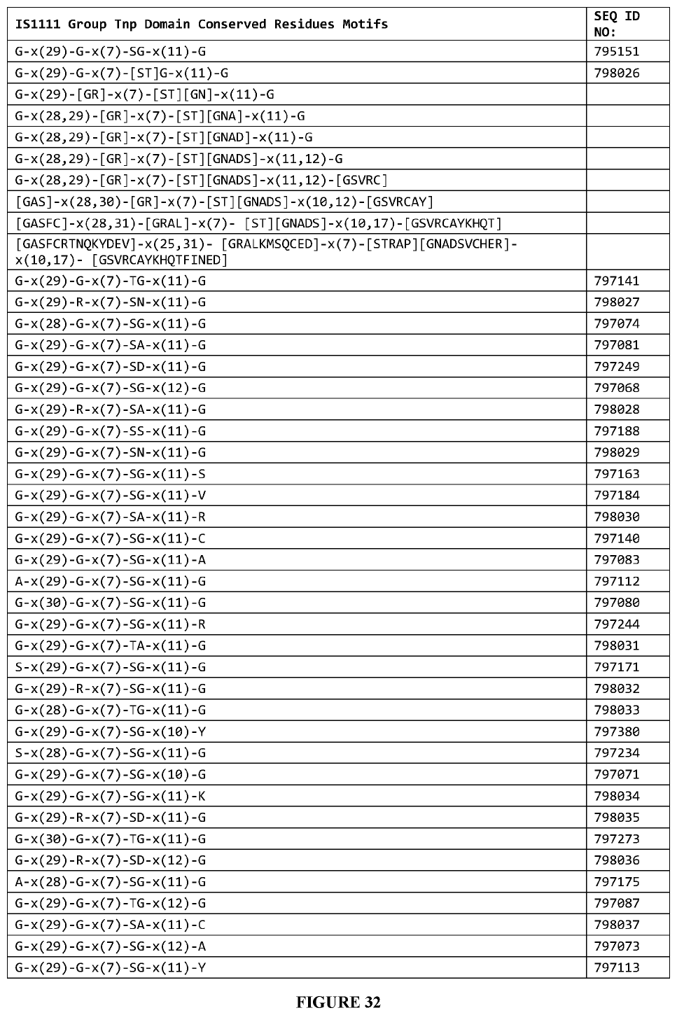

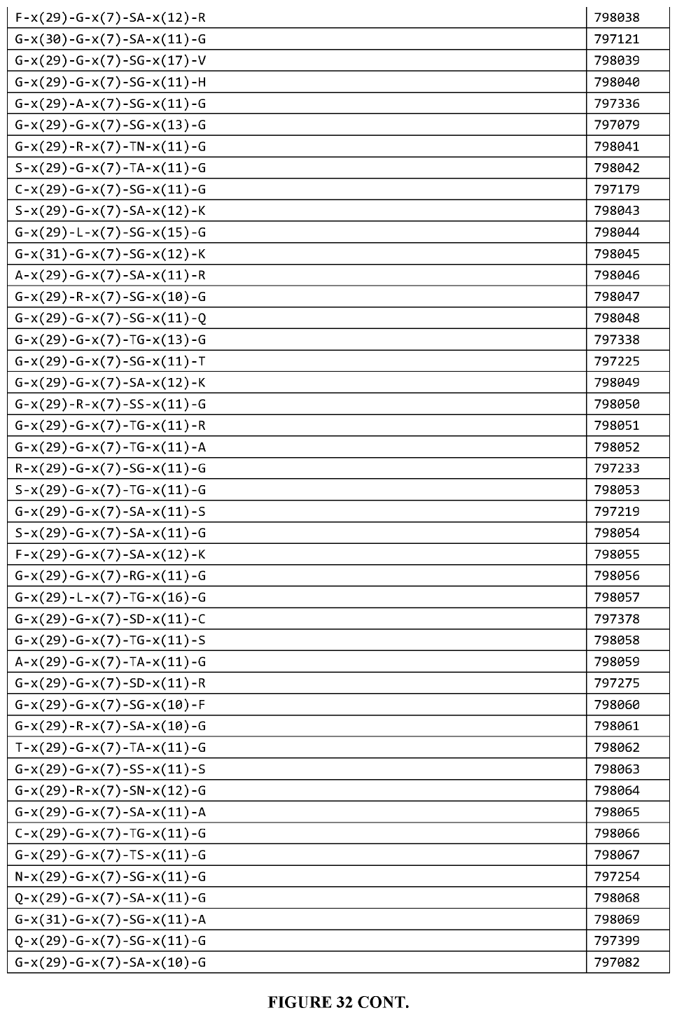

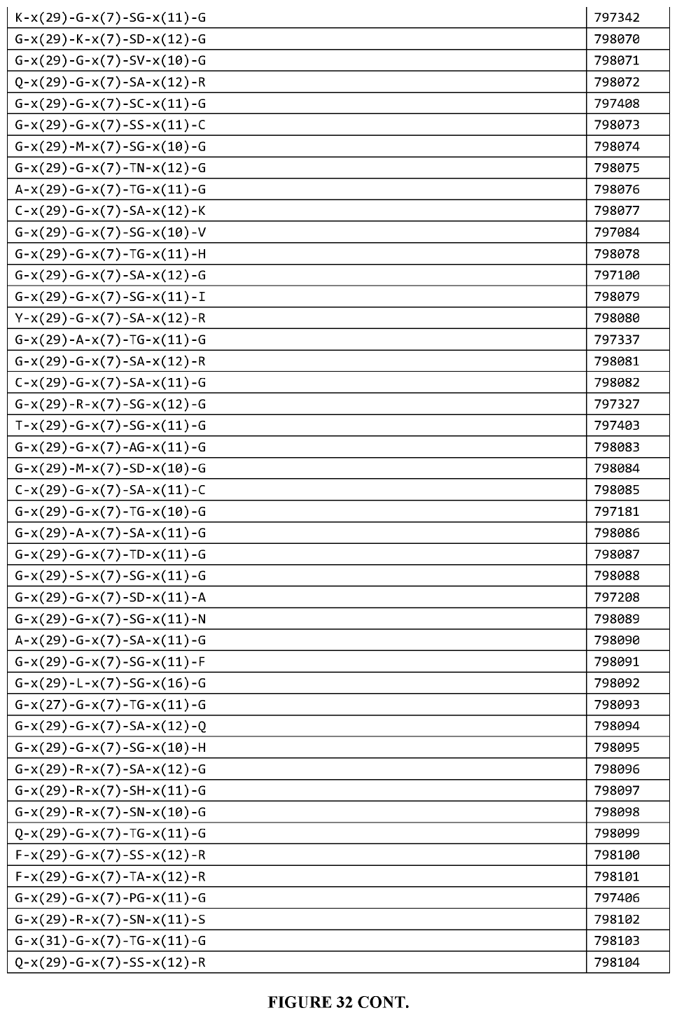

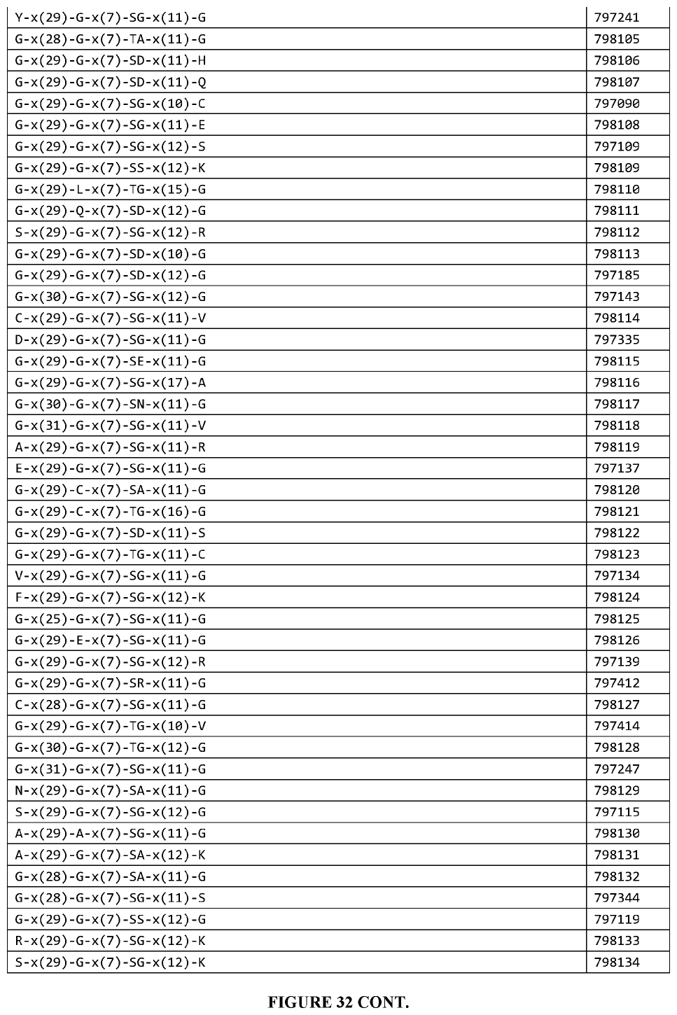

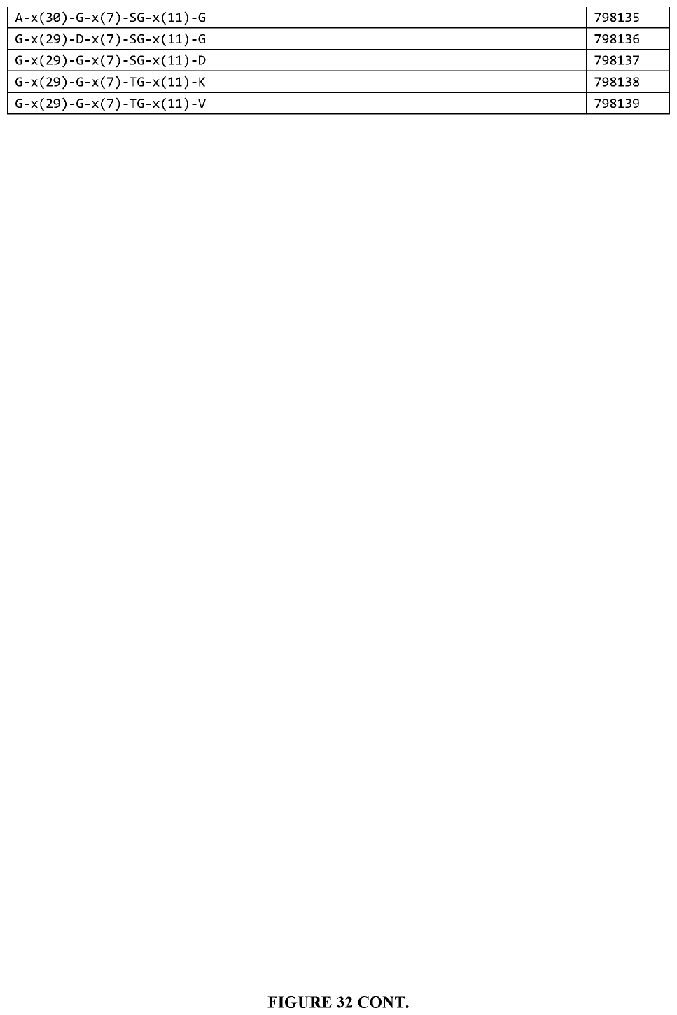

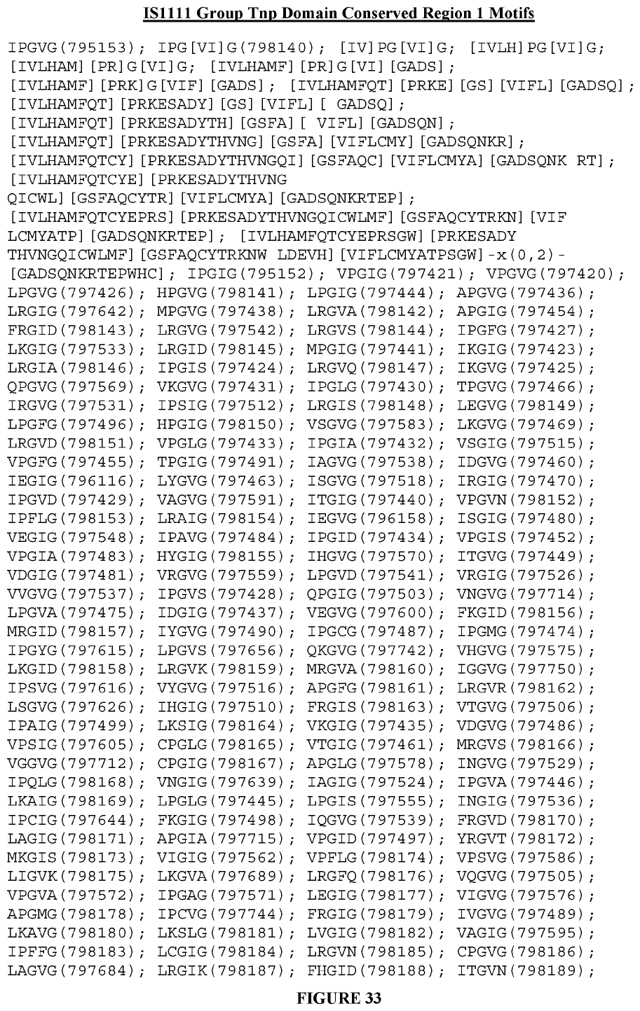

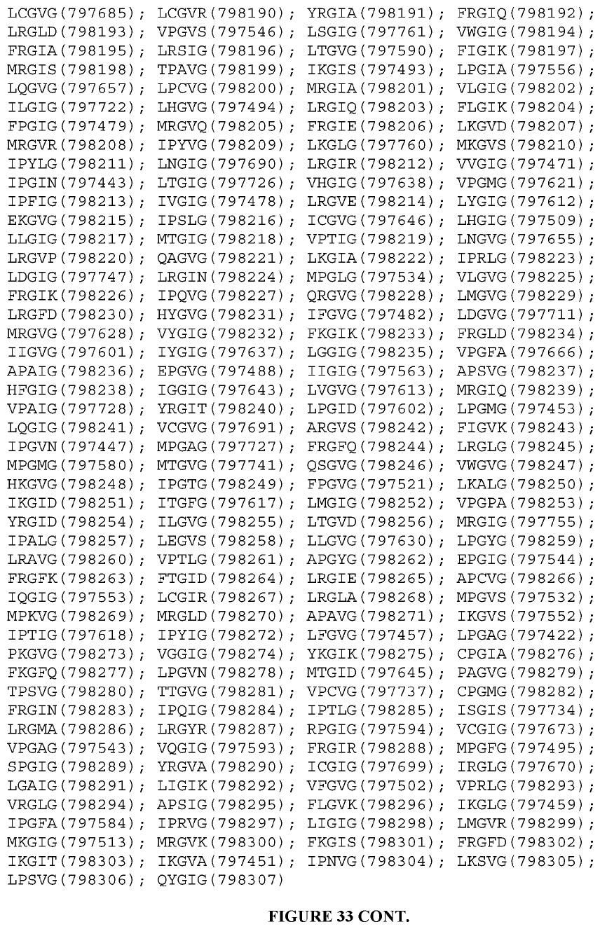

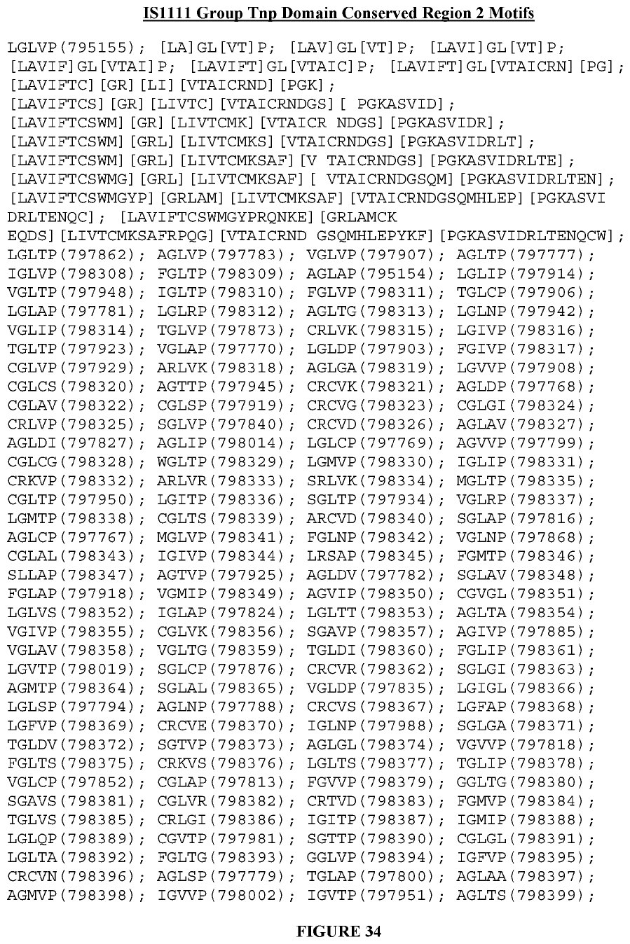

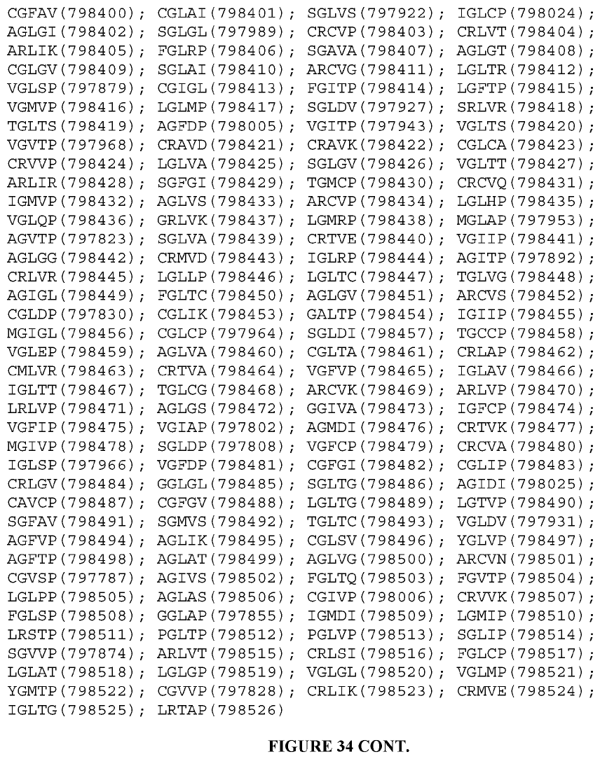

In some embodiments, the IS110 transposases belonging to the IS1111 group comprise a transposase domain comprising a domain motifs provided in FIG. 32 with motifs in common Prosite format, where x is any amino acid and x(n) represents n number of any amino acid and x(n,m) represents n to m number of any amino acids. In some embodiments, the IS110 transposases belonging to the IS1111 group comprise a transposase domain comprising one or more of the transposase domain motifs provided in FIGS. 33-34 with motifs in common Prosite format, where x is any amino acid and x(n) represents n number of any amino acid and x(n,m) represents n to m number of any amino acids.

### C.3. Linker Domain

The IS110 family transposases described herein comprise a linker domain between the RuvC-like DEDD catalytic domain and the transposase domain. In some embodiments, the linker domain comprised a coiled-coil.

In some embodiments, the IS110 family transposase comprises a linker domain comprising an amino acid sequence that is 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the linker domain sequences provided in FIG. 16 (SEQ ID NOS: 10176-10523) or SEQ ID NOS: 10524-20350 or 40357-516430.

### C.4. IS110 Transposases

In certain aspects, the invention provides an IS110 family transposase comprising an N-terminal RuvC-like DEDD catalytic domain comprising a RuvC-like DEDD catalytic domain sequence provided in FIG. 16 (SEQ ID NOS: 10176-10523) or SEQ ID NOS: 10524-20350 or 40357-516430, a linker domain comprising a coiled-coil, and a C-terminal transposase domain comprising a transposase domain sequences provided in FIG. 16 (SEQ ID NOS: 10176-10523) or SEQ ID NOS: 10524-20350 or 40357-516430. In some embodiments, the linker domain comprises a linker domain sequence provided in FIG. 16 (SEQ ID NOS: 10176-10523) or SEQ ID NOS: 10524-20350 or 40357-516430. In some embodiments, the IS110 family transposase comprises “protein_IS621” of FIG. 16 (SEQ ID NO:10176).

In certain aspects, the invention provides an IS110 family transposase consisting of an N-terminal RuvC-like DEDD catalytic domain consisting of a RuvC-like DEDD catalytic domain sequences provided in FIG. 16 (SEQ ID NOS: 10176-10523) or SEQ ID NOS: 10524-20350 or 40357-516430, a linker domain comprising a coiled-coil, and a C-terminal transposase domain consisting of a transposase domain sequences provided in FIG. 16 (SEQ ID NOS: 10176-10523) or SEQ ID NOS: 10524-20350 or 40357-516430. In some embodiments, the linker domain consists of a linker domain sequence provided in FIG. 16 (SEQ ID NOS: 10176-10523) or SEQ ID NOS: 10524-20350 or 40357-516430. In some embodiments, the IS110 family transposase consists of “protein_IS621” of FIG. 16 (SEQ ID NO:10176).

In certain aspects, the invention provides an IS110 family transposase comprising, in the N-terminal to C-terminal direction, a RuvC-like DEDD catalytic domain comprising any of the motifs or sequences in FIGS. 21-28 and 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the RuvC-like DEDD catalytic domain sequences provided in FIG. 16 (SEQ ID NOS: 10176-10523) or SEQ ID NOS: 10524-20350 or 40357-516430, a linker domain comprising a coiled-coil, and a transposase domain comprising any of the motifs or sequences in FIGS. 30-34 and 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the transposase domain sequences provided in FIG. 16 (SEQ ID NOS: 10176-10523) or SEQ ID NOS: 10524-20350 or 40357-516430. In some embodiments, the linker domain comprises an amino acid sequence 50% identical or more, 51% identical or more, 52% identical or more, 53% identical or more, 54% identical or more, 55% identical or more, 56% identical or more, 57% identical or more, 58% identical or more, 59% identical or more, 60% identical or more, 61% identical or more, 62% identical or more, 63% identical or more, 64% identical or more, 65% identical or more, 66% identical or more, 67% identical or more, 68% identical or more, 69% identical or more, 70% identical or more, 71% identical or more, 72% identical or more, 73% identical or more, 74% identical or more, 75% identical or more, 76% identical or more, 77% identical or more, 78% identical or more, 79% identical or more, 80% identical or more, 81% identical or more, 82% identical or more, 83% identical or more, 84% identical or more, 85% identical or more, 86% identical or more, 87% identical or more, 88% identical or more, 89% identical or more, 90% identical or more, 91% identical or more, 92% identical or more, 93% identical or more, 94% identical or more, 95% identical or more, 96% identical or more, 97% identical or more, 98% identical or more, 99% identical or more, or 100% identical to the linker domain sequences provided in FIG. 16 (SEQ ID NOS: 10176-10523) or SEQ ID NOS: 10524-20350 or 40357-516430.

In certain aspects, described herein is an IS110 family transposase amino acid sequence further comprising a nuclear localization signal (NLS). In some embodiments the NLS is encoded at the N-terminus of the IS110 family transposase amino acid sequence. In some embodiments the NLS is encoded at the C-terminus of the IS110 family transposase amino acid sequence. In some embodiments, the IS110 family transposase amino acid sequence further comprises more than one NLS. In some embodiments, the IS110 family transposase amino acid sequence further comprises two NLSs. In some embodiments, the IS110 family transposase amino acid sequence further comprises three NLSs. In some embodiments, the IS110 family transposase amino acid sequence further comprises more than three NLSs. In some embodiments, the IS110 family transposase amino acid sequence further comprises three NLSs at the N-terminus of the IS110 family transposase amino acid sequence. In some embodiments, the IS110 family transposase amino acid sequence further comprises three NLSs at the C-terminus of the IS110 family transposase amino acid sequence. Any NLS known in that art can be used. In some embodiments, the NLS is an SV40 NLS.

Without being bound by theory, the observed IS110 transposition reaction resembles that of conservative site-specific recombinases (CSSRs). Two DNA sequences are specifically recognized by the transposition machinery. The bridgeRNA recognizes both the donor site sequence and target site sequence; the transposase binds the bridgeRNA and, in some embodiments, STIR subsequences of the donor site. Excision of an IS110 transposon element is scarless, the original target sequence is reconstituted to its original sequence pre-insertion.

In some embodiments, the present invention could be supplemented with additional IS110 orthologs with improved efficiency and/or specificity using an extended bioinformatic search. IS110 systems with improved efficiency, such as in mammalian cells, could be identified by prioritizing candidates that occur in organisms that naturally grow at physiological temperatures. For example, candidates that naturally reside within human gut microbes could be prioritized for experimental characterization in human cells. Alternatively, IS110 systems that are found in the genomes of extremophile organisms could be prioritized for experimental characterization due to their potential utility as in vitro, temperature-responsive molecular tools. In another embodiment, IS110 systems with high specificity could be prioritized based on predicted properties of their bridgeRNA sequences, which are further described below (see Section D).

The present invention also contemplates the use of the IS110 system with cofactors and/or additional domains that may impart new functions for the IS110 system, increase the specificity of the IS110 system, and/or increase the efficiency of the IS110 system. These may exist within IS110 elements and/or in the vicinity of IS elements.

In some embodiments, the present invention contemplates an approach to engineering IS110 systems. Scientists have demonstrated that it is possible to infer the identity of an ancestral protein using phylogenetic analysis (Alonso-Lerma, B., Jabalera, Y., Samperio, S. et al., Evolution of CRISPR-associated endonucleases as inferred from resurrected proteins., Nat Microbiol 8, 77-90 (2023)). These inferred ancestral proteins may not exist in nature in the present day, but they represent a common ancestor of an existing clade of proteins. These ancestral proteins could then be experimentally synthesized and tested to determine if they have favorable properties. In one embodiment, a clade of IS110s with particular long or specific bridgeRNA target and/or donor binding loops could be analyzed using this method to identify an ancestral protein that is capable of binding to a bridgeRNA molecule with even more favorable properties.

In one embodiment, the present invention could be supplemented with additional IS110 orthologs using an extended bioinformatic search. Some IS110 transposases may have fused with other domains, imparting new biochemical properties to the IS110 system. In some embodiments, IS110 transposases with unusually long amino acid sequences could be investigated as potential candidates for these domain fusions. In some embodiments, protein domain collections, such as Pfam and InterPro, could be used to search across all proteins that are known to contain an IS110 RuvC-like DEDD catalytic domain, an IS110 transposase domain, or both. These IS110s could then be synthesized and experimentally characterized to identify any favorable properties that they may have. In another embodiment, IS110 transposases may not be fused to an additional protein cofactor as a single amino acid sequence, but they may associate with them in a multi-protein complex. These complexes could be identified by searching for proteins that occur near an IS110 CDS with high frequency in natural genomes. These additional cofactors could be synthesized and experimentally characterized to identify any favorable properties that they may have.

In some embodiments, the present invention contemplates an approach to engineering IS110 systems wherein the transposase domain is replaced or fused with a integrase, a nucleobase deaminase, a reverse transcriptase, a recombinase, an integrase, a topoisomerase, a retrotransposon, phosphatase, polymerase, a ligase, a helitron, a helicase, a methylase, a demethylase, a translation activator, a translation repressor, a transcription activator, a transcription repressor, a transcription release factor, a chromatin modifier, a histone modifier, an acetylase, a deacetylase, a reverse transcriptase, or a nuclease. Fusions with the transposase domain can comprise simply fusing one of the aforementioned effector domain-comprising enzymes or domain(s) thereof with either the N- or C-terminus of the transposase. In some embodiments, the IS110 transposase may also be modified such that the catalytic activity of the RuvC or transposase domain is crippled. In some embodiments, as an alternative to direct fusions, co-delivery of one or more of these effector domains along with, for example, an inactive IS110 transposase domain.

In some embodiments, the present invention contemplates an approach to engineering IS110 systems wherein the IS110 transposase comprises one or more amino acid mutations as compared to a wild type, whereby the mutations increase binding and/or interaction with a target site sequence, donor site sequence, and/or bridgeRNA and/or increase IS110 transposase activity

In some embodiments, the IS110 transposases may be engineered in such a way so as to reduce, limit, or eliminate the reverse reactions of insertion, excisive recombination, or inversion, in accordance with the observation that IS110 transposons excise themselves using an inefficient mechanism.

In certain aspects, described herein are nucleic acids encoding any of the IS110 family transposase amino acid sequences provided herein. In any of the embodiments described herein, a nucleotide sequence encoding the IS110 transposases or portions thereof can be codon-optimized. This type of optimization is known in the art and entails the mutation of foreign-derived DNA to mimic the codon preferences of the intended host organism or cell while encoding the same protein. Thus, the codons are changed, but the encoded protein remains unchanged. For example, if the intended target cell was a human cell, a human codon-optimized IS110 transposases or portions thereof would be a suitable IS110 transposase. As another non-limiting example, if the intended host cell were a mouse cell, then a mouse codon-optimized IS110 transposases or portions thereof would be a suitable IS110 transposase. While codon optimization is not required, it is acceptable and may be preferable in certain cases.

### D. BridgeRNA

It was bioinformatically and experimentally determined that IS110 transposases bind an RNA, referred to herein as bridgeRNA, expressed from the IS110 element non-coding ends and which directs donor and target site specificity. See Examples 1 and 3-5. BridgeRNA was previously referred to as “directing RNA” or “dRNA” and any references to directing RNA or dRNA refer to the bridgeRNA.

As discussed above, concatenation of the RE and LE may form a promoter which promotes expression of the bridgeRNA. The bridgeRNA forms a RNA-protein complex with the transposase and recognizes via base-pairing the target site or the donor site and target site together to mediate transposition. In some embodiments, the bridgeRNA can be recombinantly expressed using any suitable promoter. In some embodiments, portions of the bridgeRNA can be recombinantly expressed as two separate molecules using one promoter or multiple promoters. In some embodiments, the bridgeRNA can be recombinantly expressed using a promoter designed from the concatenation sequence of the RE and LE as described supra. In some embodiments, the bridgeRNA can be recombinantly expressed using a type III pol III promoter, which is known in the art to be suitable for small RNA expression, such as for guide RNAs in CRISPR-Cas systems. In some embodiments, the promoter is a sigma70 promoter box.

The bridgeRNA for IS110 transposases of the IS110 group is typically encoded within the LE, although in some embodiments the bridgeRNA can be at least partially encoded within RE also. In some embodiments, the bridgeRNA can be encoded within the LE and at least partially encoded within a 5′ portion of the CDS sequence encoding the IS110 transposase. The bridgeRNA for IS110 transposases of the IS110 group have donor and target binding loops to recognize the donor site sequence and the target site sequence via base-pairing to mediate transposition.

The bridgeRNA for IS110 transposases of the IS1111 group is typically encoded within the RE. In some embodiments, the bridgeRNA can be encoded within the RE and at least partially encoded within a 3′ portion of the CDS sequence encoding the IS110 transposase (e.g., an IS1111 group I110 transposase). The bridgeRNA for I110 transposases of the IS1111 group have donor and target binding loops to recognize the donor site sequence and the target site sequence via base-pairing to mediate transposition. In some embodiments, the donor and target binding sequences of the bridgeRNA for IS110 transposases of the IS1111 group can be present on a single loop of the bridgeRNA that recognizes the donor site sequence and the target site sequence via base-pairing to mediate transposition. In some embodiments, the donor binding sequences of the bridgeRNA for IS110 transposases of the IS1111 group can be present on a multi-branched loop of the bridgeRNA that recognizes the donor site sequence via base-pairing to mediate transposition.

The bridgeRNA refers to a single-stranded RNA molecule that undergoes intramolecular base pairing to form one or more stem-loop structures (which includes stem structures with external loops, bulge loops, multi-branched loops, hairpin loops and/or internal loops, see e.g., FIG. 1 of Sato, K., Akiyama, M. & Sakakibara, Y. RNA secondary structure prediction using deep learning with thermodynamic integration. Nat Commun 12, 941 (2021), doi.org/10.1038/s41467-021-21194-4, the content of which is hereby incorporated by reference in its entirety). Typically, a stem is formed by two portions of the RNA molecule which are complementary when read in opposite directions and base pair to form a stem with intervening unpaired nucleotides between the two complementary portions forming a loop. A stem sequence does not need to be 100% complementary and can comprise mismatches, bulges, or internal loops.

In some embodiments, the bridgeRNA comprises a RNA molecule that comprises at least one stem-loop structure and further comprises one or more loops comprising a first nucleotide sequence that is complementary to a first target site sequence of a target DNA, a second nucleotide sequence that is complementary to a second target site sequence which is on the opposite strand of the target DNA to the first target site sequence, a third nucleotide sequence that is complementary to a first donor site sequence of a donor DNA, and a fourth nucleotide sequence that is complementary to a second donor site sequence which is on the opposite strand of the donor DNA to the first donor site sequence. In some embodiments, the bridgeRNA comprises a first internal loop comprising a first nucleotide sequence that is complementary to a first target site sequence of a target DNA, a second nucleotide sequence that is complementary to a second target site sequence which is on the opposite strand of the target DNA to the first target site sequence, and a second internal loop comprising a third nucleotide sequence that is complementary to a first donor site sequence of a donor DNA, and a fourth nucleotide sequence that is complementary to a second donor site sequence which is on the opposite strand of the donor DNA to the first donor site sequence. In some embodiments, the bridgeRNA comprises an internal loop comprising a first nucleotide sequence that is complementary to a first target site sequence of a target DNA, a second nucleotide sequence that is complementary to a second target site sequence which is on the opposite strand of the target DNA to the first target site sequence, and a multi-branched loop comprising a third nucleotide sequence that is complementary to a first donor site sequence of a donor DNA, and a fourth nucleotide sequence that is complementary to a second donor site sequence which is on the opposite strand of the donor DNA to the first donor site sequence. In some embodiments, said bridgeRNA binds a IS110 group transposase. In some embodiments, said bridgeRNA binds a IS1111 group transposase. In some embodiments, said bridgeRNA binds the transposase of IS621, ISPa11, IsPa29, ISMmg1, ISPfl1, ISMae40, ISStma6, ISAzs32, ISMex9, ISCARN28, ISAar16, ISCps7, ISPpu9, ISRe19, ISEsa2, ISMma5, IS900, or ISHne5. In some embodiments, said bridgeRNA binds the transposase of IS621. In some embodiments, a loop comprising the first and second nucleotide sequences that are complementary to the target site sequence of a target DNA may form a stem or partial stem structure when not bound by a transposase and form a single stranded structure when bound to a transposase. In some embodiments, a loop comprising the third and fourth nucleotide sequences that are complementary to the donor site sequence of a donor DNA may form a stem or partial stem structure when not bound by a transposase and form a single stranded structure when bound to a transposase.

With reference to the above bridgeRNA, in some embodiments, the first and second nucleotide sequences are fully complementary to their respective target site sequences of the target DNA. In some embodiments, the first and/or second nucleotide sequences are partially complementary to their respective target site sequences of the target DNA, i.e., there is non-canonical base pairing, mismatch and/or non-contiguous tolerance. In some embodiments, there is a single mismatch in the first or second nucleotide sequence. In some embodiments, there are two, three or four mismatches which can be in the first nucleotide sequence, in the second nucleotide sequence, or spread across the first and second nucleotide sequences. In some embodiments, the two, three or four mismatches are contiguous. In some embodiments, the two, three or four mismatches are non-contiguous. In some embodiments, there is a single non-canonical base pair in the first or second nucleotide sequence. In some embodiments, there are two, three or four non-canonical base pairs which can be in the first nucleotide sequence, in the second nucleotide sequence, or spread across the first and second nucleotide sequences. In some embodiments, the two, three or four non-canonical base pairs are contiguous. In some embodiments, the two, three or four non-canonical base pairs are non-contiguous. In some embodiments, the third and fourth nucleotide sequences are fully complementary to their respective donor site sequences of the donor DNA. In some embodiments, the third and/or fourth nucleotide sequences are partially complementary to their respective donor site sequences of the donor DNA, i.e., there is non-canonical base pairing, mismatch and/or non-contiguous tolerance. In some embodiments, there is a single mismatch in the third or fourth nucleotide sequence. In some embodiments, there are two mismatches which can be in the third nucleotide sequence, in the fourth nucleotide sequence, or spread across the third and fourth nucleotide sequences. In some embodiments, the two mismatches are contiguous. In some embodiments, the two mismatches are non-contiguous. In some embodiments, there is a single non-canonical base pair in the third or fourth nucleotide sequence. In some embodiments, there are two non-canonical base pairs which can be in the third nucleotide sequence, in the fourth nucleotide sequence, or spread across the third and fourth nucleotide sequences. In some embodiments, the two non-canonical base pairs are contiguous. In some embodiments, the two non-canonical base pairs are non-contiguous. In some embodiments, the non-canonical base pairing is non-Watson-Crick base pairing (i.e., is not a G-C base pair or A-T/U base pair). For example, see Pacesa M, Lin C H, Cléry A, et al., Structural basis for Cas9 off-target activity, Cell, 2022, 185(22):4067-4081.e21, the contents of which is hereby incorporated by reference in its entirety). In some embodiments, the non-canonical base pairing is wobble base pairing. In some embodiments, the non-canonical base pairing is Hoogsteen base pairing. In some embodiments, the non-canonical base pairing comprises a rG-dT base pair, a rU-dG base pair, a rA-dC base pair, a rC-dA base pair, a rA-dG base pair, or a rG-dG base pair.

In some embodiments, the bridgeRNA comprises a RNA molecule that comprises at least one stem-loop structure and further comprises one or more loops comprising a first nucleotide sequence that is complementary to a first target site sequence of a target DNA, a second nucleotide sequence that is complementary to a second target site sequence which is on the opposite strand of the target DNA to the first target site sequence. In some embodiments, the bridgeRNA further comprises a third nucleotide sequence that is complementary to a first donor site sequence of a donor DNA, and a fourth nucleotide sequence that is complementary to a second donor site sequence which is on the opposite strand of the donor DNA to the first donor site sequence. In some embodiments, the bridgeRNA comprises a first internal loop comprising a first nucleotide sequence that is complementary to a first target site sequence of a target DNA, a second nucleotide sequence that is complementary to a second target site sequence which is on the opposite strand of the target DNA to the first target site sequence. In some embodiments, the bridgeRNA comprises a second internal loop comprising a third nucleotide sequence that is complementary to a first donor site sequence of a donor DNA, and a fourth nucleotide sequence that is complementary to a second donor site sequence which is on the opposite strand of the donor DNA to the first donor site sequence. In some embodiments, the first internal loop (e.g. a multi-branched loop) of the bridgeRNA comprises a third nucleotide sequence that is complementary to a first donor site sequence of a donor DNA, and a fourth nucleotide sequence that is complementary to a second donor site sequence which is on the opposite strand of the donor DNA to the first donor site sequence. In some embodiments, said bridgeRNA binds an IS1111 group transposase. In some embodiments, said bridgeRNA binds the transposase of IS1111_229727. In some embodiments, a loop comprising the first and second nucleotide sequences that are complementary to the target site sequence of a target DNA may form a stem or partial stem structure when not bound by a transposase and form a single stranded structure when bound to a transposase. In some embodiments, a loop comprising the third and fourth nucleotide sequences that are complementary to the donor site sequence of a donor DNA may form a stem or partial stem structure when not bound by a transposase and form a single stranded structure when bound to a transposase.

With reference to the above bridgeRNA, in some embodiments, the first and second nucleotide sequences are fully complementary to their respective target site sequences of the target DNA. In some embodiments, the first and/or second nucleotide sequences are partially complementary to their respective target site sequences of the target DNA, i.e., there is non-canonical base pairing, mismatch and/or non-contiguous tolerance. In some embodiments, there is a single mismatch in the first or second nucleotide sequence. In some embodiments, there are two, three or four mismatches which can be in the first nucleotide sequence, in the second nucleotide sequence, or spread across the first and second nucleotide sequences. In some embodiments, the two, three or four mismatches are contiguous. In some embodiments, the two, three or four mismatches are non-contiguous. In some embodiments, there is a single non-canonical base pair in the first or second nucleotide sequence. In some embodiments, there are two, three or four non-canonical base pairs which can be in the first nucleotide sequence, in the second nucleotide sequence, or spread across the first and second nucleotide sequences. In some embodiments, the two, three or four non-canonical base pairs are contiguous. In some embodiments, the two, three or four non-canonical base pairs are non-contiguous. In some embodiments, the third and fourth nucleotide sequences are fully complementary to their respective donor site sequences of the donor DNA. In some embodiments, the third and/or fourth nucleotide sequences are partially complementary to their respective donor site sequences of the donor DNA, i.e., there is non-canonical base pairing, mismatch and/or non-contiguous tolerance. In some embodiments, there is a single mismatch in the third or fourth nucleotide sequence. In some embodiments, there are two mismatches which can be in the third nucleotide sequence, in the fourth nucleotide sequence, or spread across the third and fourth nucleotide sequences. In some embodiments, the two mismatches are contiguous. In some embodiments, the two mismatches are non-contiguous. In some embodiments, there is a single non-canonical base pair in the third or fourth nucleotide sequence. In some embodiments, there are two non-canonical base pairs which can be in the third nucleotide sequence, in the fourth nucleotide sequence, or spread across the third and fourth nucleotide sequences. In some embodiments, the two non-canonical base pairs are contiguous. In some embodiments, the two non-canonical base pairs are non-contiguous. In some embodiments, the non-canonical base pairing is non-Watson-Crick base pairing (i.e., is not a G-C base pair or A-T/U base pair). In some embodiments, the non-canonical base pairing is wobble base pairing. In some embodiments, the non-canonical base pairing is Hoogsteen base pairing. In some embodiments, the non-canonical base pairing comprises a rG-dT base pair, a rU-dG base pair, a rA-dC base pair, a rC-dA base pair, a rA-dG base pair, or a rG-dG base pair.

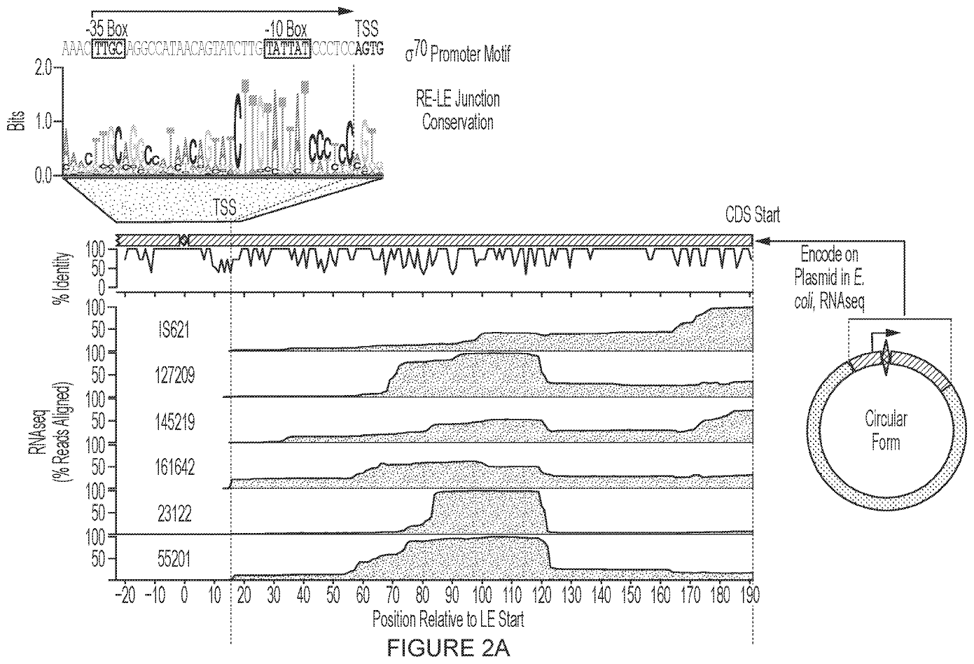

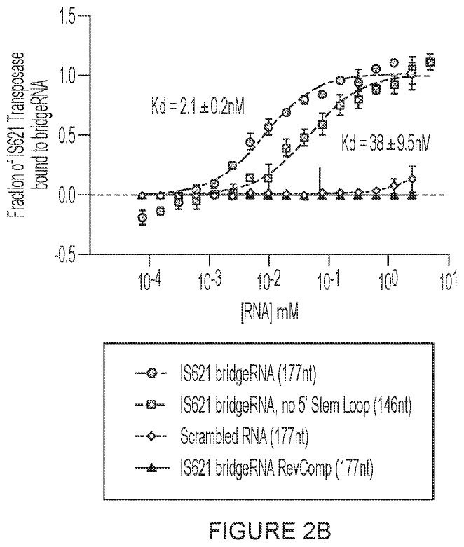

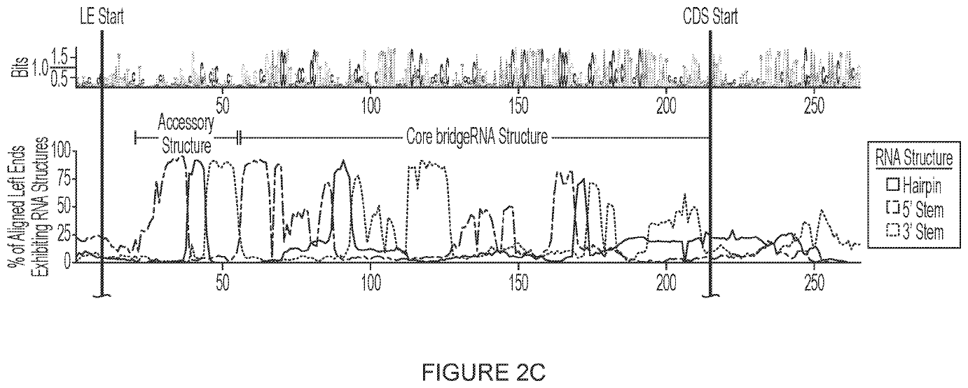

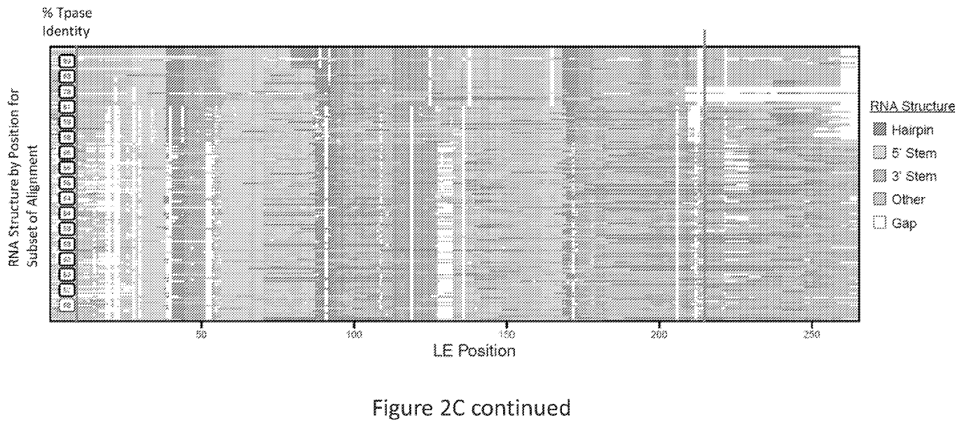

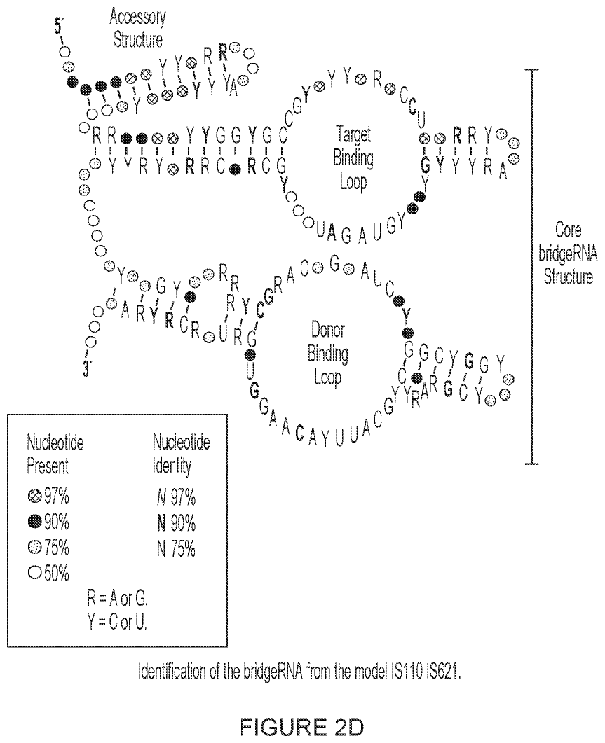

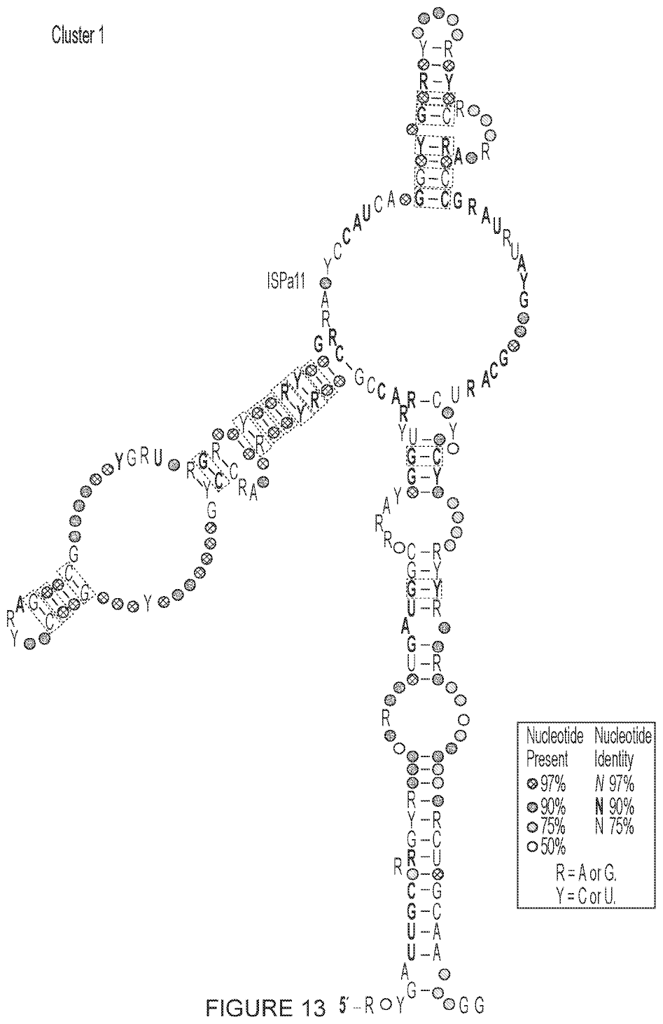

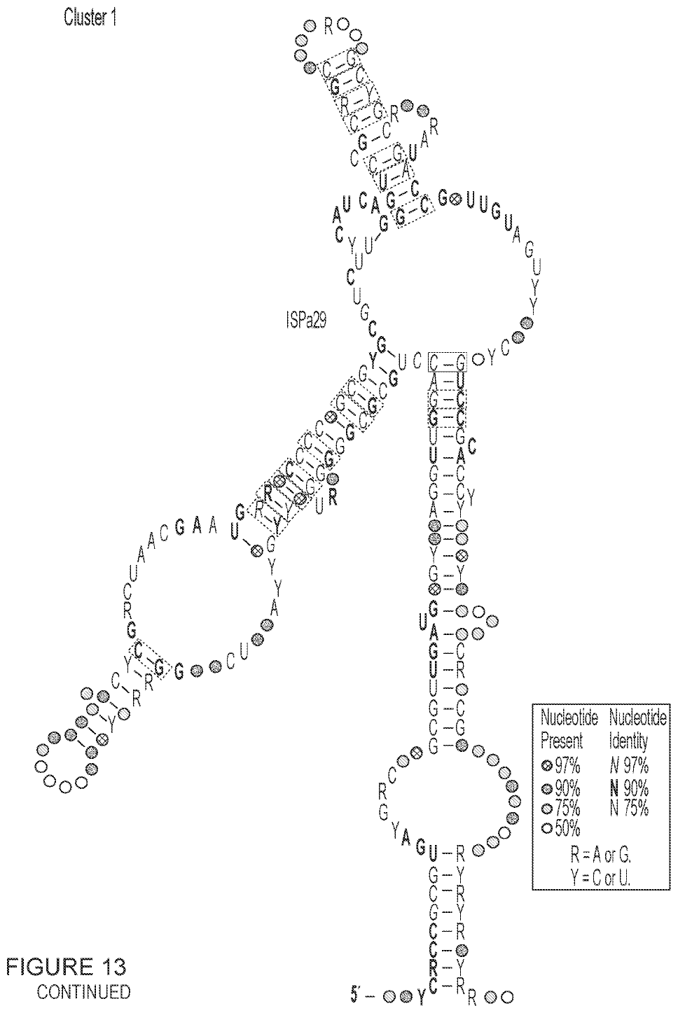

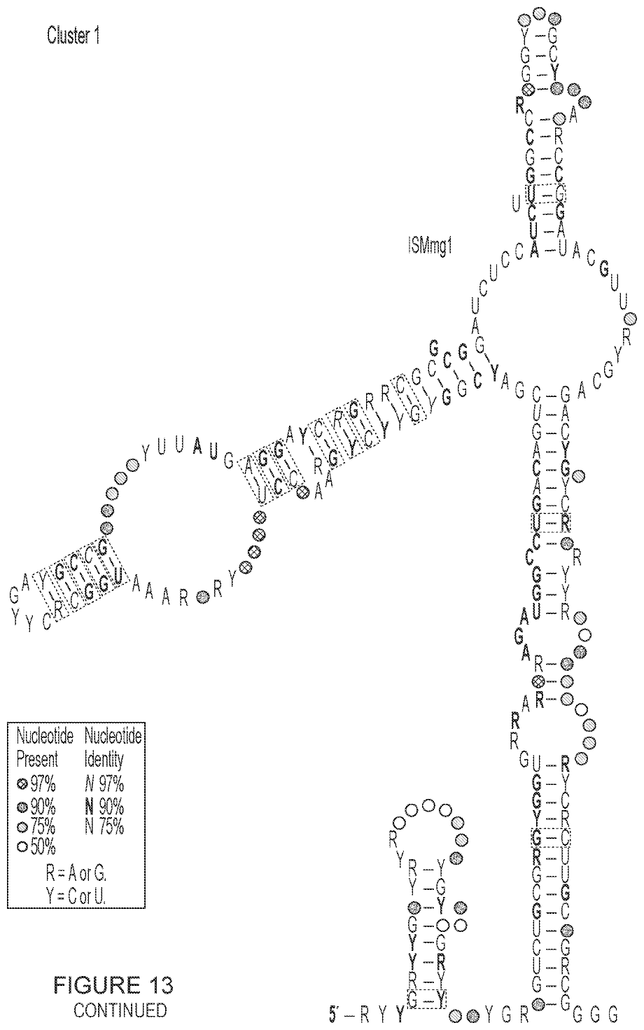

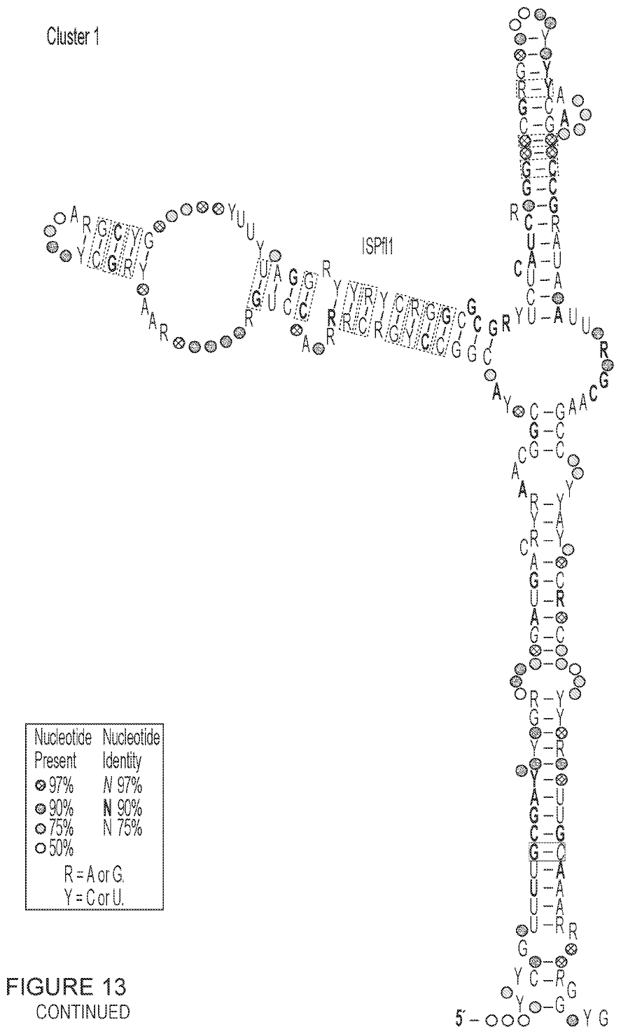

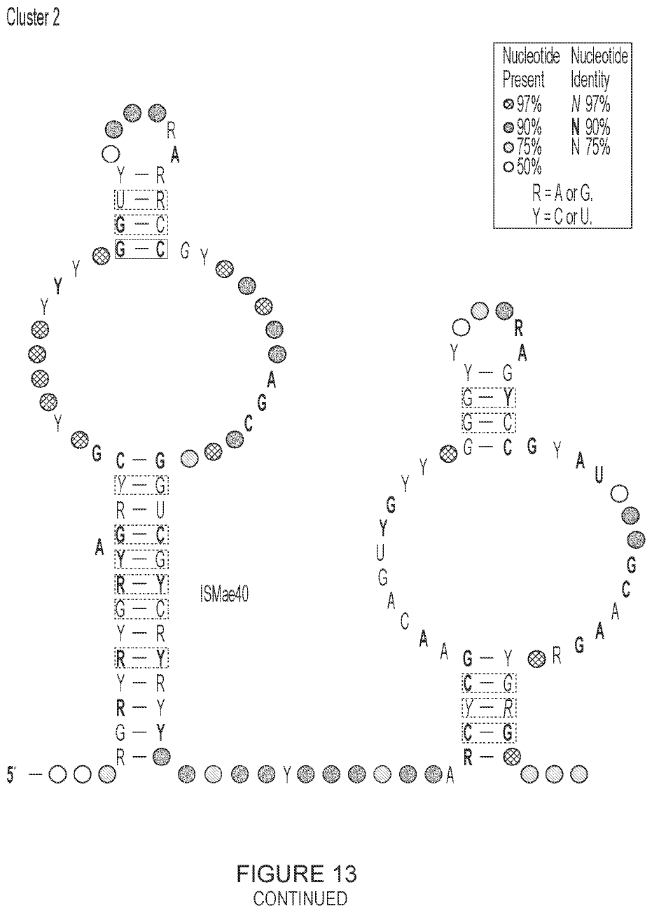

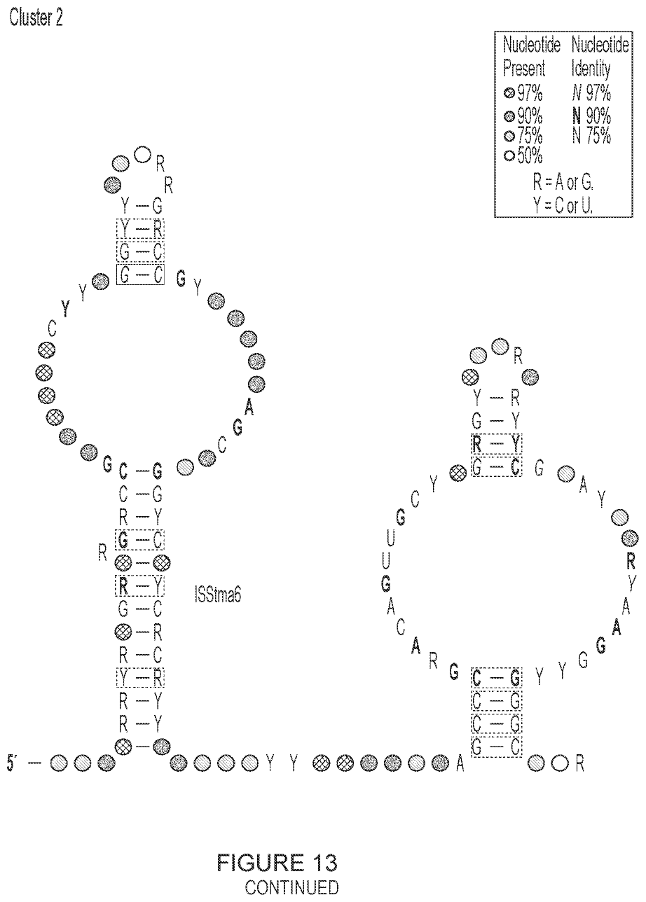

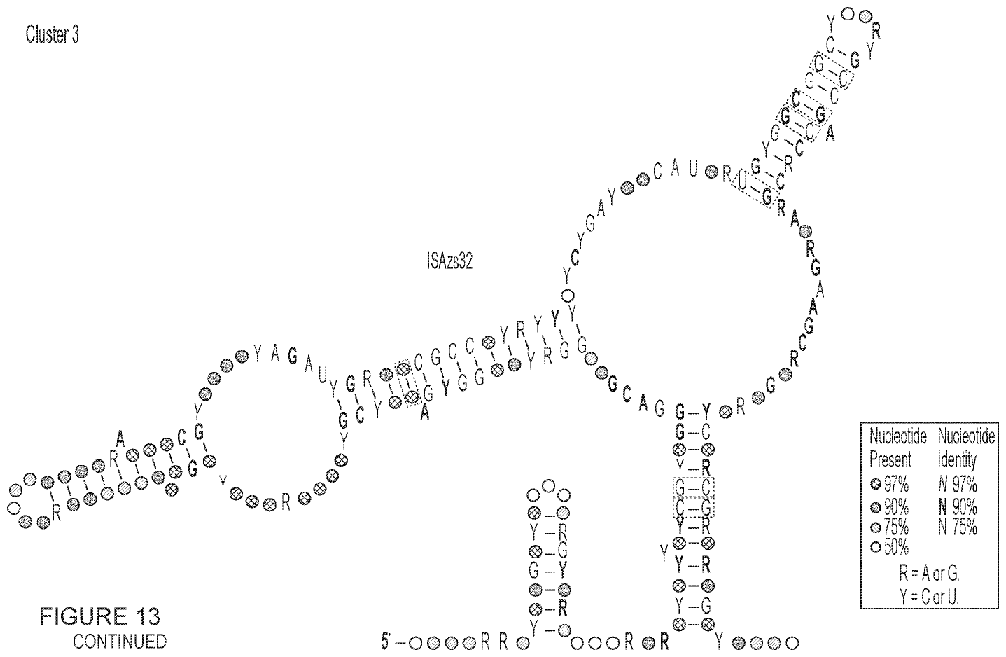

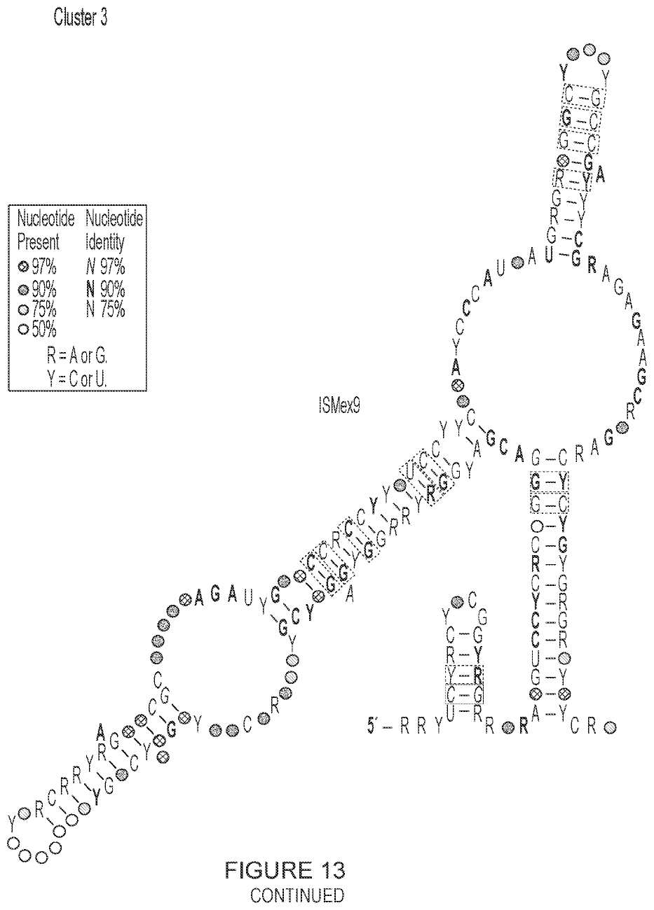

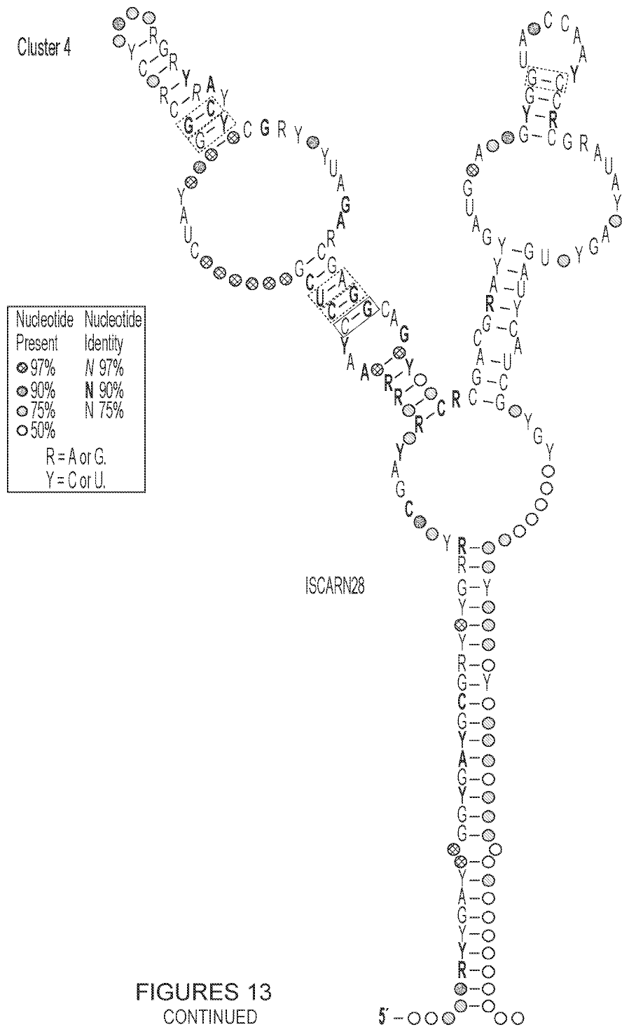

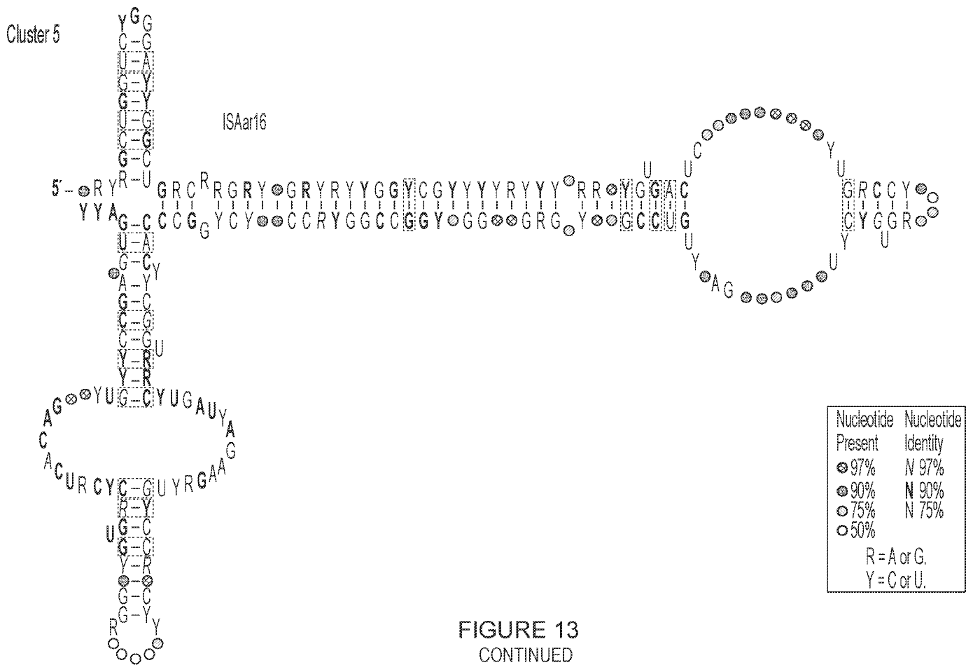

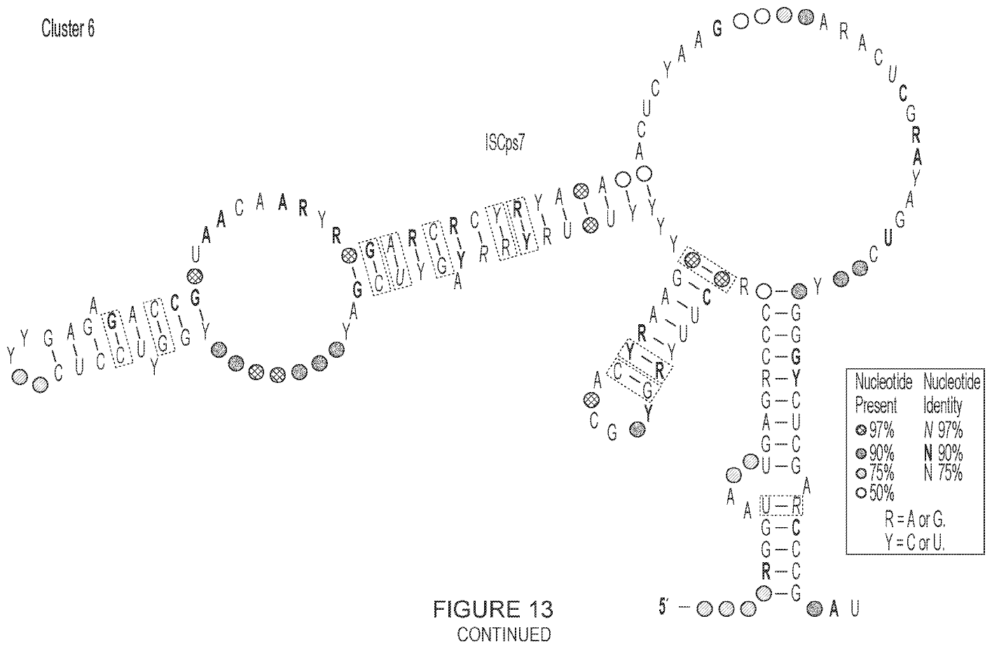

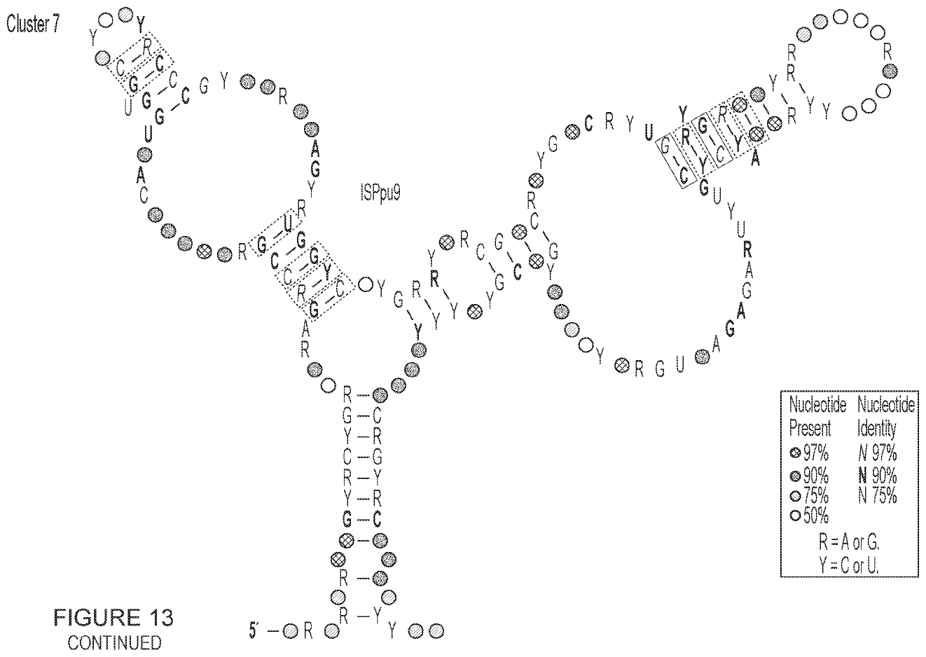

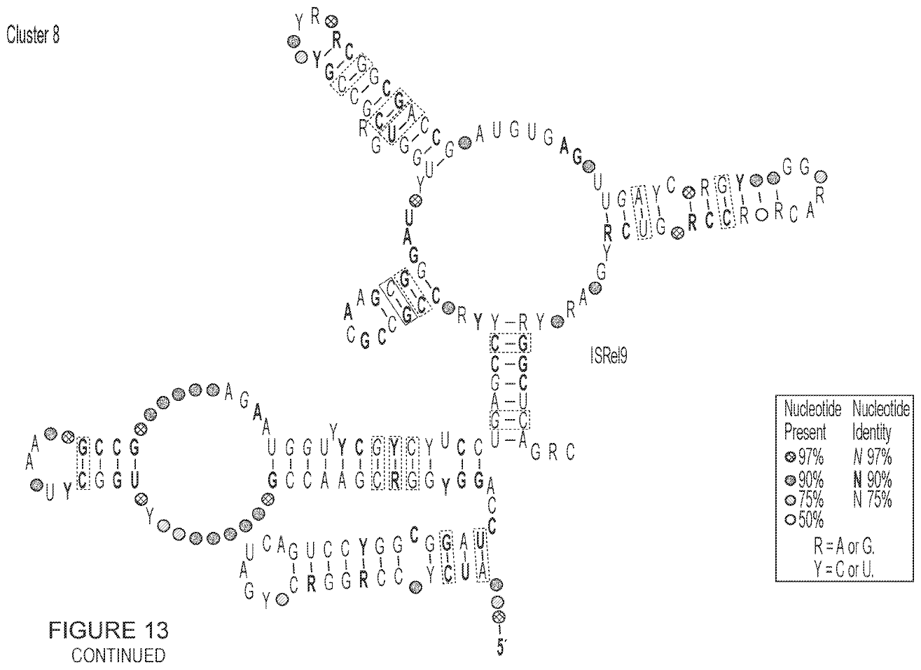

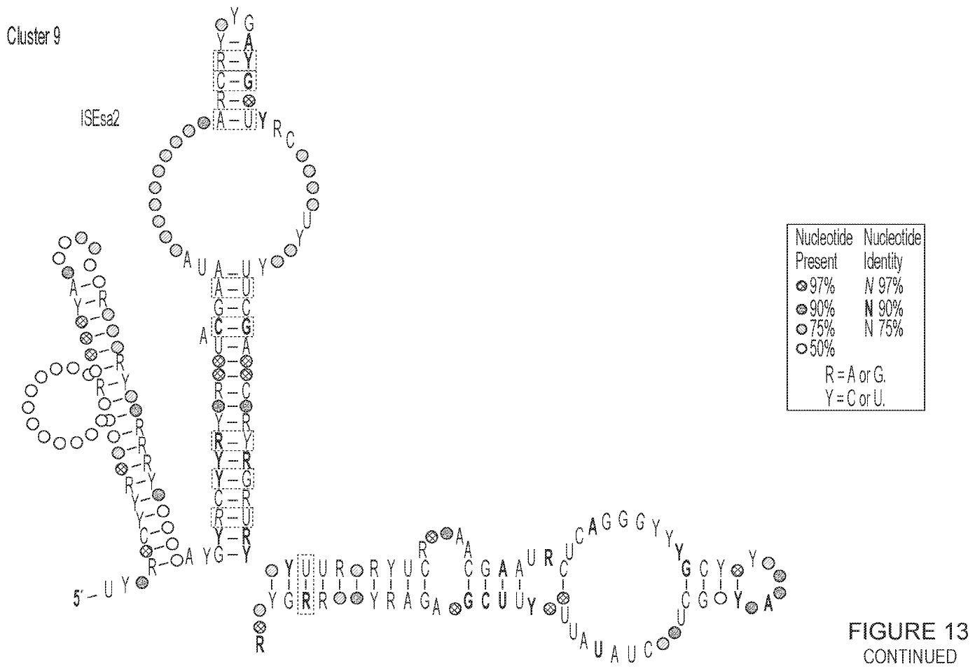

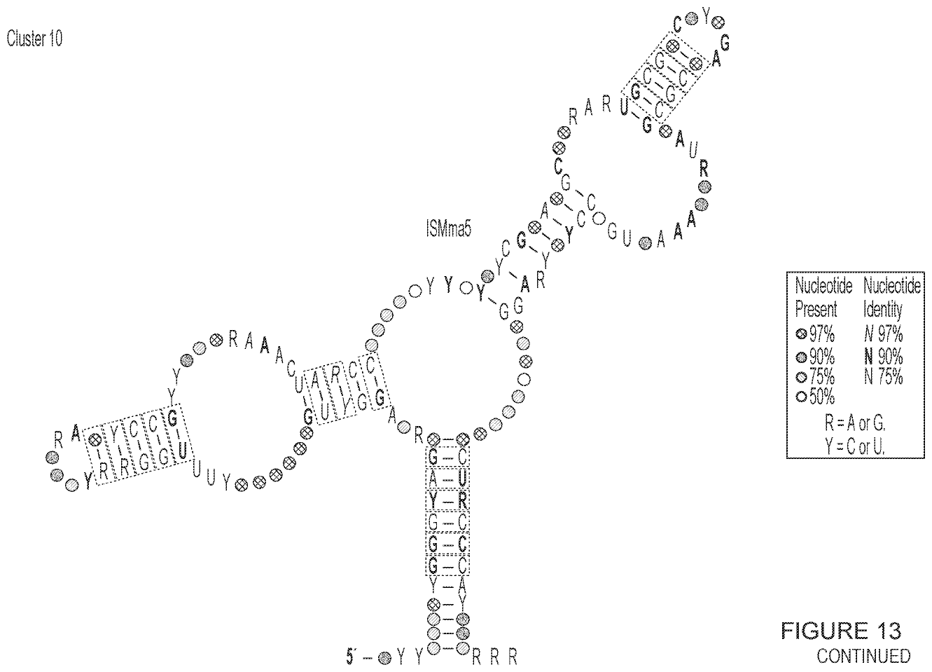

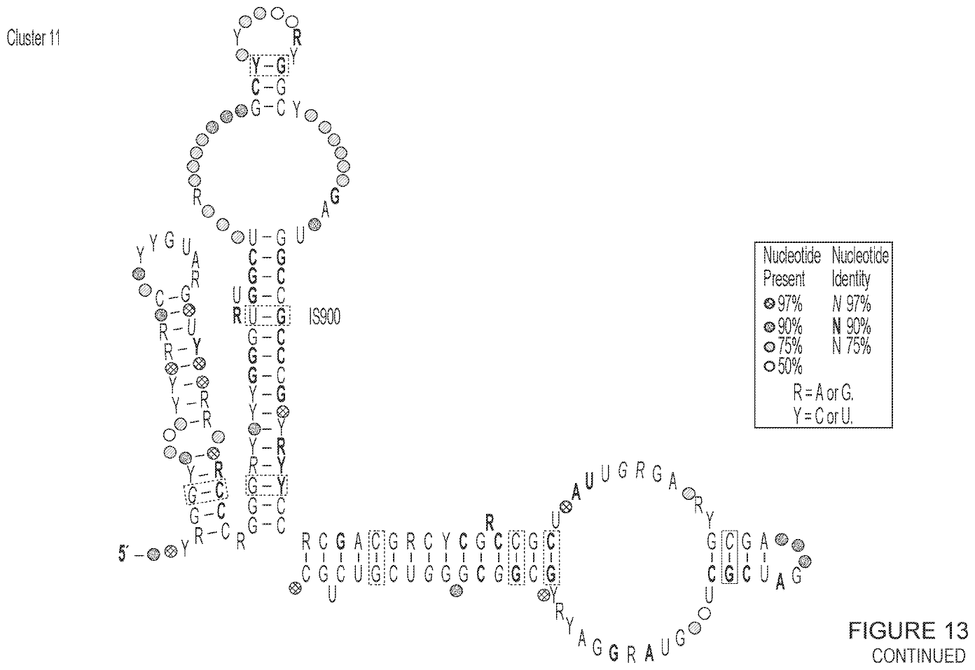

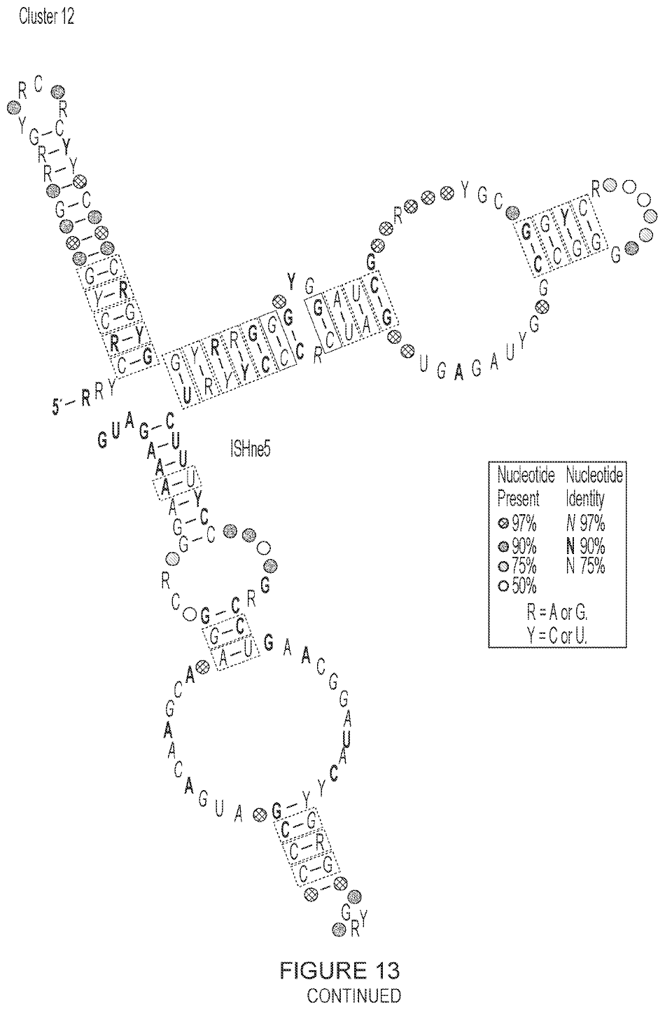

In some embodiments, the bridgeRNA comprises a RNA molecule that comprises at least two stem-loop structures, and with respect to the bridgeRNA having at least two stem-loop structures, these two structures are referred to as the “first stem-loop” and the “second stem-loop” where the first is 5′ or upstream to the second. In some embodiments, the first stem-loop comprises a target binding loop and the second stem-loop comprises a donor binding loop. In some embodiments, the first stem loop comprises a donor binding loop and the second stem-loop comprises a target binding loop. Thus, with this nomenclature, there can be additional stem-loop structures upstream of the first stem-loop, between the first and second stem-loops, and/or after the second-stem loop. In some embodiments, the bridgeRNA comprises at least a first internal loop referred to as a target binding loop and a second internal loop referred to as a donor binding loop. In some embodiments, the bridgeRNA comprises a RNA molecule that comprises at least two stem-loop structures as depicted in FIG. 2D and for Cluster 1 in FIG. 13. In FIG. 2D and for Cluster 1 in FIG. 13, and in some embodiments, the first stem-loop structure of the bridgeRNA comprises a first stem-loop (5-35 nt, 3-10 nt loop) comprising an internal loop (e.g., a target binding loop) (5-20 nt). In FIG. 2D and for Cluster 1 in FIG. 13, and in some embodiments, the second stem-loop structure of the bridgeRNA comprises a second stem-loop (5-35 nt, 3-10 nt loop) comprising an internal loop (e.g., a donor binding loop) (5-20 nt). The stem of the second stem-loop structure can include additional loops and bubbles that are 1-10 nucleotides each. As shown in FIG. 2D and in some embodiments, the bridgeRNA may further comprise an additional third stem-loop structure (5-15 nt stem, 3-10 nt loop) (accessory structure) 5′ to the first stem-loop. In some embodiments, said bridgeRNA binds the transposase of IS621. In some embodiments, said bridgeRNA binds the transposase of ISMae40 or ISStma6. In some embodiments, the donor binding loop and/or target binding loop may form a stem or partial stem structure when not bound by a transposase and form a single stranded structure when bound to a transposase.

Thus, in some embodiments, the bridgeRNA comprises a nucleotide sequence comprising the following secondary structure: first stem-loop comprising an internal target binding loop—second stem-loop comprising an internal donor binding loop. In some embodiments, the bridgeRNA comprises additional stem-loop structures, bulges, and/or loops (see, e.g., FIG. 2D). In some embodiments, the bridgeRNA comprises a nucleotide sequence comprising 5′-[A]-[B]-[C]-[D]-[E]-[F]-[G]-[H]-[I]-[J]-[K]-[L]-[M]-[N]-3′, wherein A is a first stem portion, B is a first side of an internal loop corresponding to a target binding loop, C is a second stem portion, D is a first loop portion, E is the reverse complement of C, F is a second side of the internal loop corresponding to the target binding loop, G is the reverse complement of A, H is a third stem portion, I is a first side of an internal loop corresponding to a donor binding loop, J is a fourth stem portion, K is a second loop portion, L is the reverse complement of J, M is a second side of the internal loop corresponding to the donor binding loop, and N is the reverse complement of H. In some embodiments, the reverse complement portions are not 100% complementary so that the stem structures may comprise one or more mismatches or bulges, or non-standard base-pairing may occur. In some embodiments, the first side of the internal loop corresponding to the target bind loop comprises a nucleotide sequence that is complementary to a first target site sequence of a target DNA (referred to as LTG (left target guide)) and the second side of the internal loop corresponding to the target bind loop comprises a second nucleotide sequence that is complementary to a second target site sequence which is on the opposite strand of the target DNA to the first target site sequence (referred to as RTG (right target guide)). In some embodiments, the first side of the internal loop corresponding to the donor binding loop comprises a nucleotide sequence that is complementary to a first donor site sequence of a donor DNA (referred to as LDG (left donor guide)) and the second side of the internal loop corresponding to the donor bind loop comprises a second nucleotide sequence that is complementary to a second donor site sequence which is on the opposite strand of the donor DNA to the first target site sequence (referred to as RDG (right donor guide)). In some embodiments, the stem structures may comprise one or more mismatches or bulges. In some embodiments, at least two portions of nucleotide sequence N are not complementary to the nucleotide sequence of portion H, so that the stem structure formed by base pairing between portion H and N comprises two bulges. In some embodiments, additional nucleotides are present between any of the portions A to N. For example, one or more non-base-paired nucleotides are present between portions G and H. In another embodiment, one or more nucleotides are present between the 5′ terminus and portion A. In another embodiment, one or more nucleotides are present between the 3′ terminus and portion N. In some embodiments, said bridgeRNA binds the transposase of IS621. In some embodiments, said bridgeRNA binds the transposase of ISMae40 or ISStma6. In some embodiments, the donor binding loop and/or target binding loop may form a stem or partial stem structure when not bound by a transposase and form a single stranded structure when bound to a transposase.

In some embodiments, the bridgeRNA comprises a nucleotide sequence comprising the following secondary structure: third stem-loop—first stem-loop comprising an internal target binding loop second stem-loop comprising an internal donor binding loop. In some embodiments, the bridgeRNA comprises additional stem-loop structures, bulges, and/or loops (see, e.g., FIG. 2D). In some embodiments, the bridgeRNA comprises a nucleotide sequence comprising 5′-[Z]-[X]—[Y]-[A]-[B]-[C]-[D]-[E]-[F]-[G]-[H]-[I]-[J]-[K]-[L]-[M]-[N]-3′, wherein Z is a fifth stem portion, X is a third loop portion, Y is the reverse complement of Z, A is a first stem portion, B is a first side of an internal loop corresponding to a target binding loop, C is a second stem portion, D is a first loop portion, E is the reverse complement of C, F is a second side of the internal loop corresponding to the target binding loop, G is the reverse complement of A, H is a third stem portion, I is a first side of an internal loop corresponding to a donor binding loop, J is a fourth stem portion, K is a second loop portion, L is the reverse complement of J, M is a second side of the internal loop corresponding to the donor binding loop, and N is the reverse complement of H. In some embodiments, the reverse complement portions are not 100% complementary so that the stem structures may comprise one or more mismatches or bulges, or non-standard base-pairing may occur. In some embodiments, the first side of the internal loop corresponding to the target bind loop comprises a nucleotide sequence that is complementary to a first target site sequence of a target DNA (referred to as LTG) and the second side of the internal loop corresponding to the target bind loop comprises a second nucleotide sequence that is complementary to a second target site sequence which is on the opposite strand of the target DNA to the first target site sequence (referred to as RTG). In some embodiments, the first side of the internal loop corresponding to the donor bind loop comprises a nucleotide sequence that is complementary to a first donor site sequence of a donor DNA (referred to as LDG) and the second side of the internal loop corresponding to the donor bind loop comprises a second nucleotide sequence that is complementary to a second donor site sequence which is on the opposite strand of the donor DNA to the first target site sequence (referred to as RDG). In some embodiments, the stem structures may comprise one or more mismatches or bulges. In some embodiments, at least two portions of nucleotide sequence N are not complementary to the nucleotide sequence of portion H, so that the stem structure formed by base pairing between portion H and N comprises two bulges. In some embodiments, additional nucleotides are present between any of the portions A to N. For example, one or more non-base-paired nucleotides are present between portions G and H. In another embodiment, one or more nucleotides are present between portions Y and A. In another embodiment, one or more nucleotides are present between the 5′ terminus and portion Z. In another embodiment, one or more nucleotides are present between the 3′ terminus and portion N. In some embodiments, said bridgeRNA binds the transposase of IS621. In some embodiments, the donor binding loop and/or target binding loop may form a stem or partial stem structure when not bound by a transposase and form a single stranded structure when bound to a transposase.

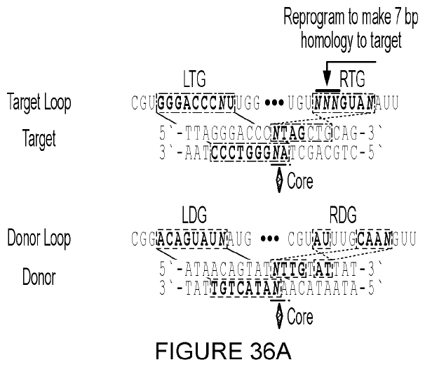

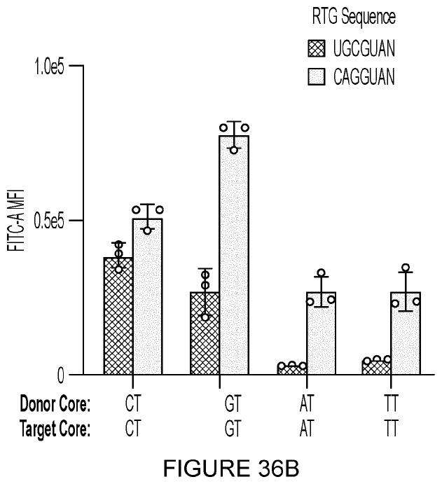

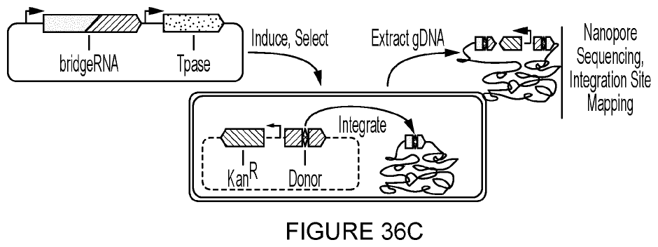

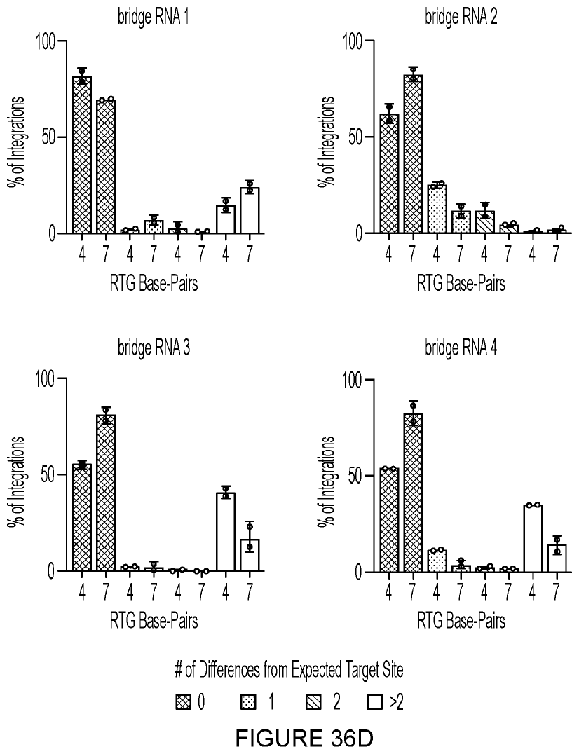

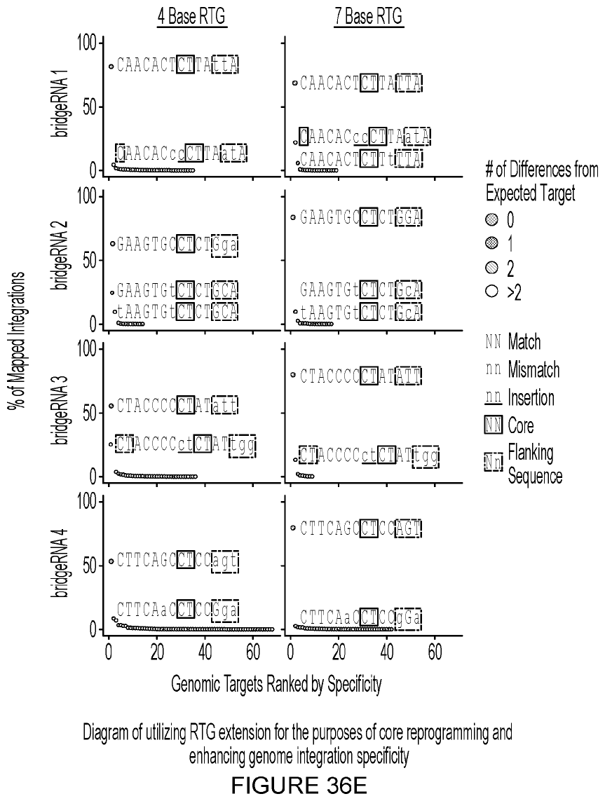

With reference to the above bridgeRNAs, in some embodiments, the LTG and RTG sequences are fully complementary to their respective target site sequences of the target DNA. In some embodiments, the LTG and/or RTG are partially complementary to their respective target site sequences of the target DNA, i.e., there is non-canonical base pairing, mismatch and/or non-contiguous tolerance. In some embodiments, there is a single mismatch in the LTG or RTG nucleotide sequence. In some embodiments, there are two, three or four mismatches which can be in the LTG, in the RTG, or spread across the LTG and RTG. In some embodiments, the two, three or four mismatches are contiguous. In some embodiments, the two, three or four mismatches are non-contiguous. In some embodiments, the LTG and/or RTG are partially complementary to their respective target site sequences of the target DNA so that there are one, two, three, or four nucleotides dispersed within LTG and/or RTG that do not base pair with the target DNA and form bulges. In some embodiments, there is a single non-canonical base pair in the LTG or RTG nucleotide sequence. In some embodiments, there are two, three or four non-canonical base pairs which can be in the LTG, in the RTG, or spread across the LTG and RTG. In some embodiments, the two, three or four non-canonical base pairs are contiguous. In some embodiments, the two, three or four non-canonical base pairs are non-contiguous. In some embodiments, the LDG and RDG nucleotide sequences are fully complementary to their respective donor site sequences of the donor DNA. In some embodiments, the LDG and/or RDG nucleotide sequences are partially complementary to their respective donor site sequences of the donor DNA, i.e., there is non-canonical base pairing, mismatch and/or non-contiguous tolerance. In some embodiments, there is a single mismatch in the LDG or RDG nucleotide sequence. In some embodiments, there are two, three or four mismatches which can be in the LDG, in the RDG, or spread across the LDG and RDG. In some embodiments, the two, three, or four mismatches are contiguous. In some embodiments, the two, three, or four mismatches are non-contiguous. In some embodiments, the LDG and/or RDG are partially complementary to their respective donor site sequences of the donor DNA so that there are one, two, three, or four nucleotides dispersed within LDG and/or RDG that do not base pair with the donor DNA and form bulges (see e.g., FIG. 36A). In some embodiments, there is a single non-canonical base pair in the LDG or RDG nucleotide sequence. In some embodiments, there are two, three, or four non-canonical base pairs which can be in the LDG, in the RDG, or spread across the LDG and RDG. In some embodiments, the two, three, or four non-canonical base pairs are contiguous. In some embodiments, the two, three, or four non-canonical base pairs are non-contiguous. In some embodiments, the non-canonical base pairing is non-Watson-Crick base pairing (i.e., is not a G-C base pair or A-T/U base pair). In some embodiments, the non-canonical base pairing is wobble base pairing. In some embodiments, the non-canonical base pairing is Hoogsteen base pairing. In some embodiments, the non-canonical base pairing comprises a rG-dT base pair, a rU-dG base pair, a rA-dC base pair, a rC-dA base pair, a rA-dG base pair, or a rG-dG base pair.

In some embodiments, the bridgeRNA comprises a nucleotide sequence comprising the sequence 5′--nnnnnnnnYYnRRnn----nnYYYnnnYnnnnRRnnnnYYGGAYGCCGYnYYnRnCCUnnRRYnnnARYYYGYnnYGU AGAUnnnYGCRnC-RRnYRYYnnnnnnnnYnnGYnnnRRRYCGRACnGnAUCnYnGGCYGGY-nnnYCGRnARYCYGCAUUYACAAGGUnGRUnRCRYRAnnn-n3′ (SEQ ID NO: 795156), wherein “n” represents any nucleotide, “R” represents an A or G nucleotide, and “Y” represents a C or U nucleotide. In some embodiments, the bridgeRNA comprises a nucleotide sequence comprising the sequence 5′--nnnnnnnnYYnRRnn----nnYYYnnnYnnnnRRnnnnYYGGAYGCCGYnYYnRnCCUnnRRYnnnARYYYGYnnYGU AGAUnnnYGCRnC-RRnYRYYnnnnnnnnYnnGYnnnRRRYCGRACnGnAUCnYnGGCYGGY-nnnYCGRnARYCYGCAUUYACAAGGUnGRUnRCRYRAnnn-n3′ (SEQ ID NO: 795156), wherein “n” represents any nucleotide, “R” represents an A or G nucleotide, and “Y” represents a C or U nucleotide and wherein the bridgeRNA comprises the secondary structure 5′........((((((..........)))))).....((((((((.(((............(((((....)))))..............))))).)))))))............................... ....(((((.....)))))..................................3′, wherein matching parentheses “(“and”)” indicate base-paired nucleotides, and “.” indicate unpaired bases. In some embodiments, the bridgeRNA comprises a nucleotide sequence comprising the sequence 5′--nnnnnnnnYYnRRnn----nnYYYnnnYnnnnRRnnnnYYGGAYGCCGYnYYnRnCCUnnRRYnnnARYYYGYnnYGU AGAUnnnYGCRnC-RRnYRYYnnnnnnnnYnnGYnnnRRRYCGRACnGnAUCnYnGGCYGGY-nnnYCGRnARYCYGCAUUYACAAGGUnGRUnRCRYRAnnn-n3′ (SEQ ID NO: 795156), wherein “n” represents any nucleotide, “R” represents an A or G nucleotide, and “Y” represents a C or U nucleotide and wherein the bridgeRNA comprises the secondary structure 5′.....((((((((((........)))))))))).((((((((((.(((............(((((....)))))..............))))).)))))))).......(.((((....(((.... .........((((((.....)))))...)...............)))..)))).)....3′, wherein matching parentheses “(“and”)” indicate base-paired nucleotides, and “.” indicate unpaired bases. In some embodiments, the bridgeRNA comprises a nucleotide sequence comprising the sequence 5′--nnnnnnnnYYnRRnn----nnYYYnnnYnnnnRRnnnnYYGGAYGCCGYnYYnRnCCUnnRRYnnnARYYYGYnnYGU AGAUnnnYGCRnC-RRnYRYYnnnnnnnnYnnGYnnnRRRYCGRACnGnAUCnYnGGCYGGY-nnnYCGRnARYCYGCAUUYACAAGGUnGRUnRCRYRAnnn-n3′ (SEQ ID NO: 795156), wherein “n” represents any nucleotide, “R” represents an A or G nucleotide, and “Y” represents a C or U nucleotide and wherein the bridgeRNA comprises the secondary structure 5′.....(((((((((((......))))))))))).((((((((((.(((...(((.((...(((((....)))))..))...)))....))))).))))))))......((((((((..((((. ..........((((((((.....)))))...))).............))))))))))))...3′, wherein matching parentheses “(“and”)” indicate base-paired nucleotides, and “.” indicate unpaired bases. In some embodiments, the bridgeRNA comprises a nucleotide sequence comprising the sequence 5′--nnnnnnnnYYnRRnn----nnYYYnnnYnnnnRRnnnnYYGGAYGCCGYnYYnRnCCUnnRRYnnnARYYYGYnnYGU AGAUnnnYGCRnC-RRnYRYYnnnnnnnnYnnGYnnnRRRYCGRACnGnAUCnYnGGCYGGY-nnnYCGRnARYCYGCAUUYACAAGGUnGRUnRCRYRAnnn-n3′ (SEQ ID NO: 795156), wherein “n” represents any nucleotide, “R” represents an A or G nucleotide, and “Y” represents a C or U nucleotide and wherein the bridgeRNA comprises the secondary structure 5′.....(((((((((((......))))))))))).((((((((((.(((...((((((...(((((....)))))..)))..)))....))))).))))))))...((.((((((((..(((((.((.((. ...((((((((.....)))))...))).....))...))))))))))))))))).3′, wherein matching parentheses “(” and “)” indicate base-paired nucleotides, and “.” indicate unpaired bases. In some embodiments, said bridgeRNA binds transposase IS621.

In some embodiments, the bridgeRNA comprises at least a target binding loop and a donor binding loop and is encoded by a sequence that is at least 50%, 55%, 60%, 65%, 70%, 75%, 80%, 85%, 90%, 91%, 92%, 93%, 94%, 95%, 96%, 97%, 98% or 99% identical to a bridgeRNA sequence of FIG. 15 (SEQ ID NOS: 1-348) or SEQ ID NOS: 349-10175. In some embodiments, the bridgeRNA comprises a sequence that is at least 50%, 55%, 60%, 65%, 70%, 75%, 80%, 85%, 90%, 91%, 92%, 93%, 94%, 95%, 96%, 97%, 98% or 99% identical to a bridgeRNA sequence of FIG. 15 (SEQ ID NOS: 1-348) or SEQ ID NOS: 349-10175.

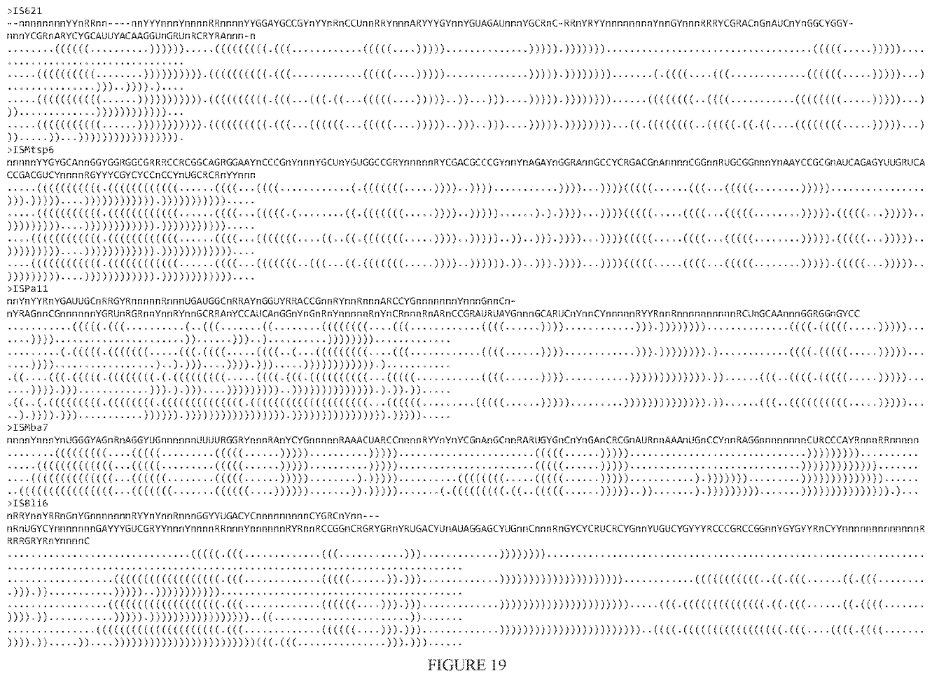

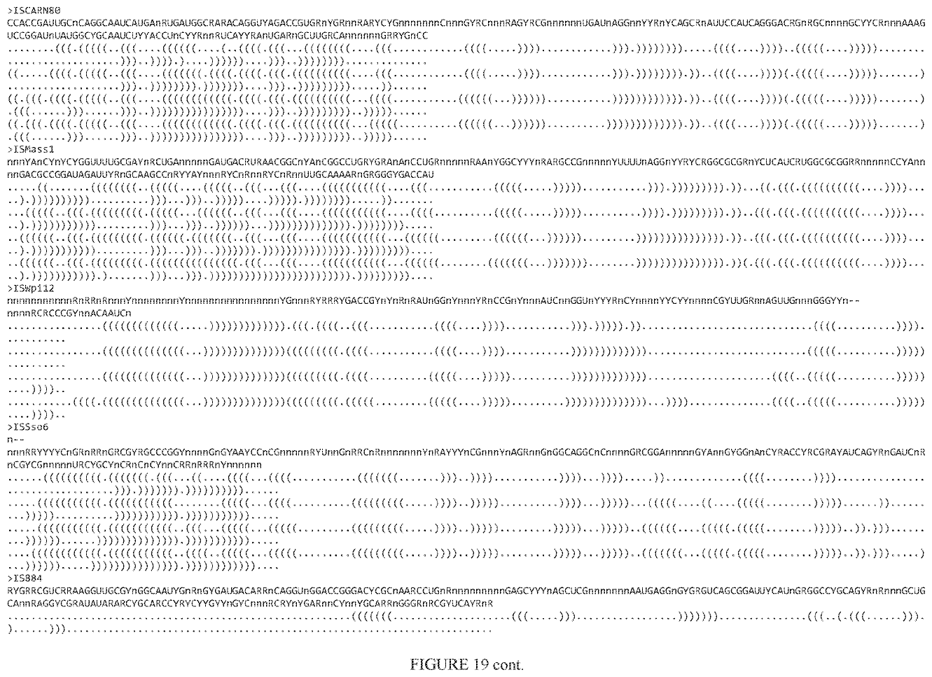

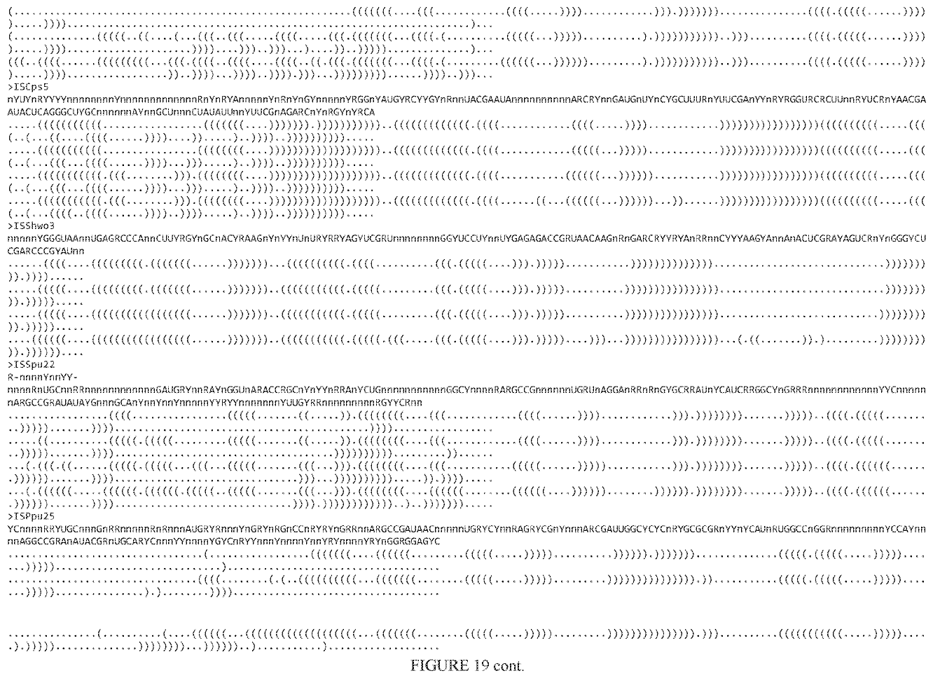

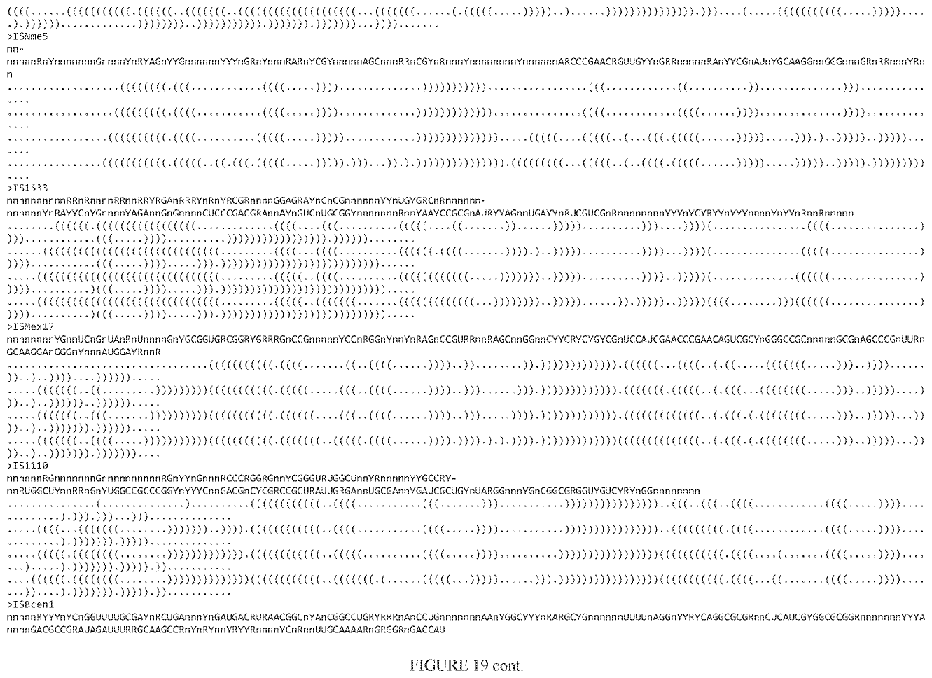

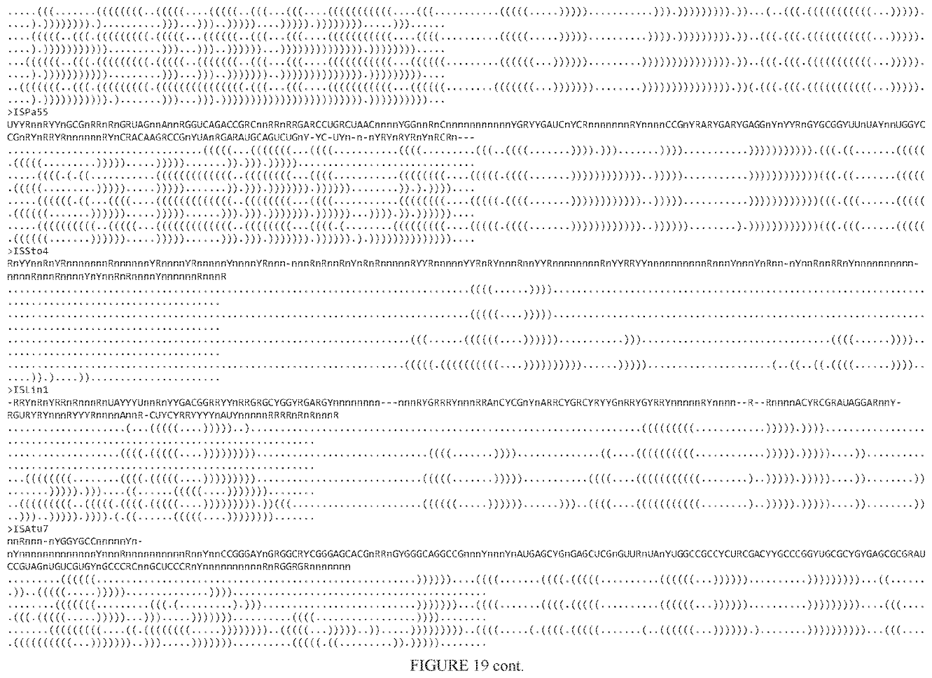

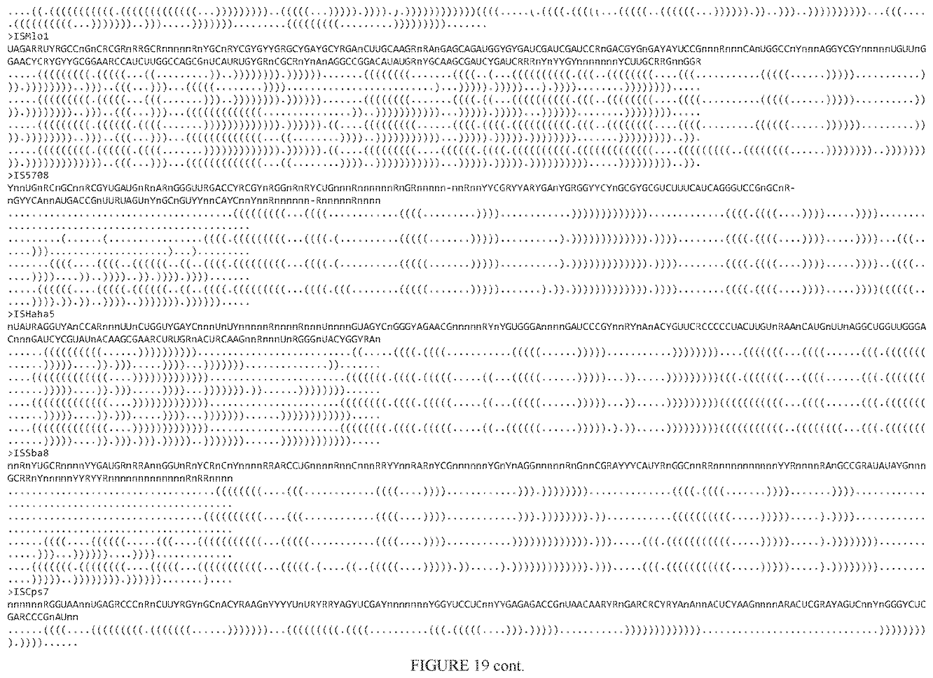

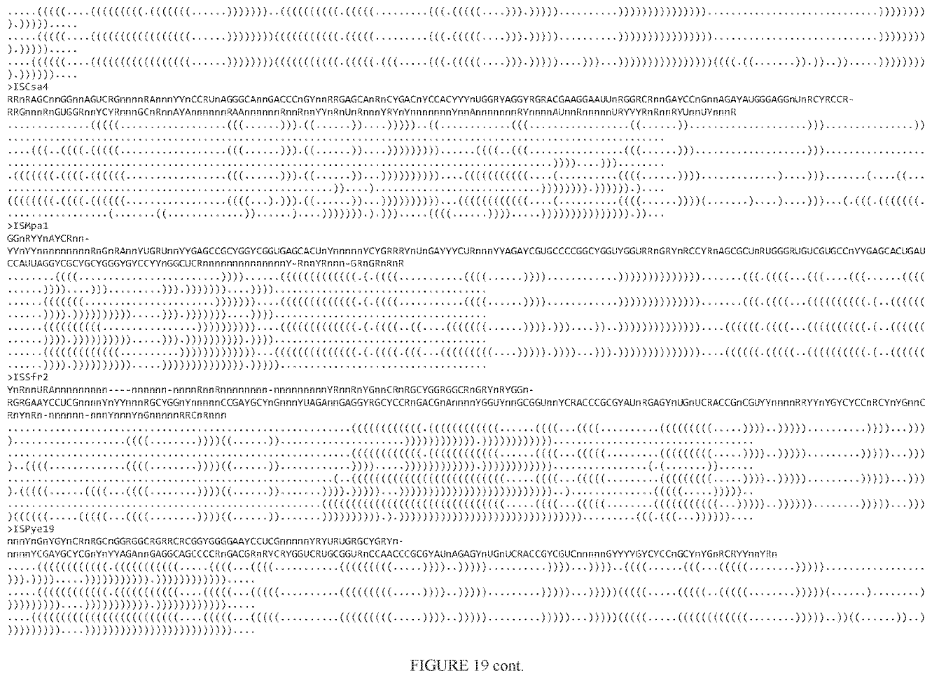

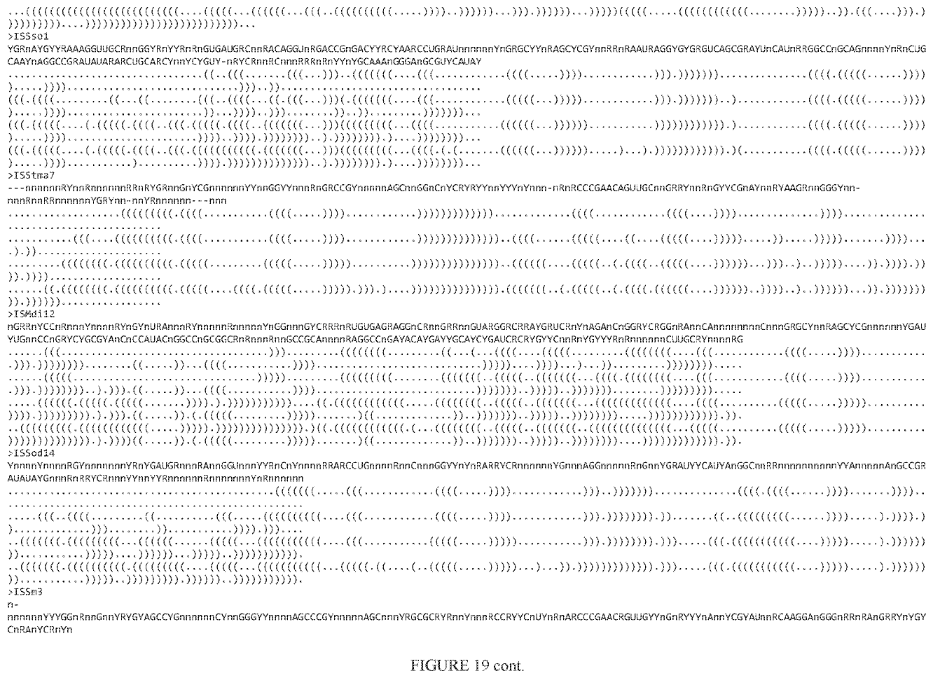

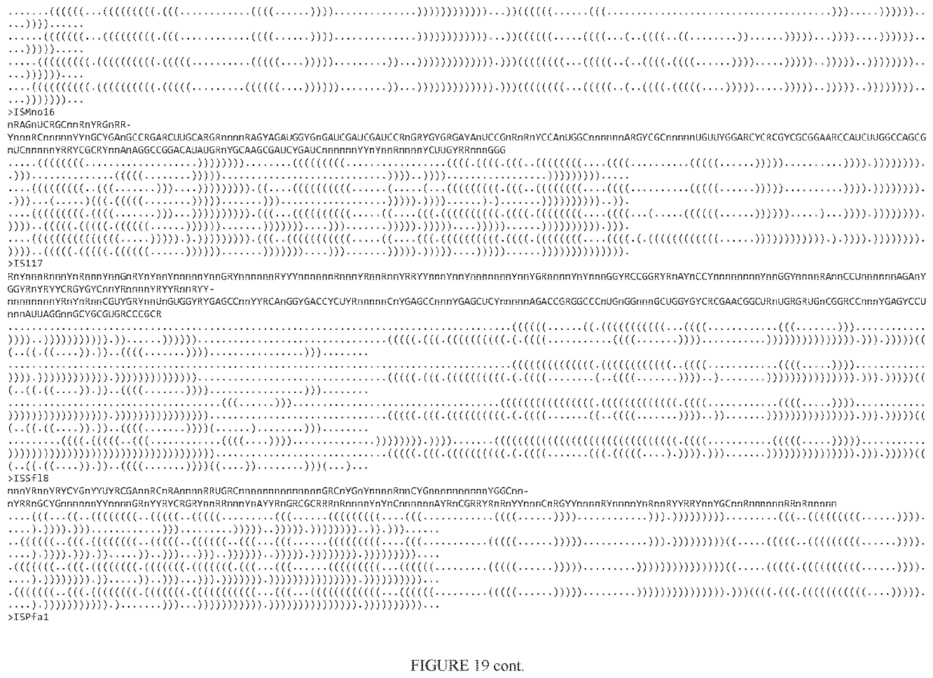

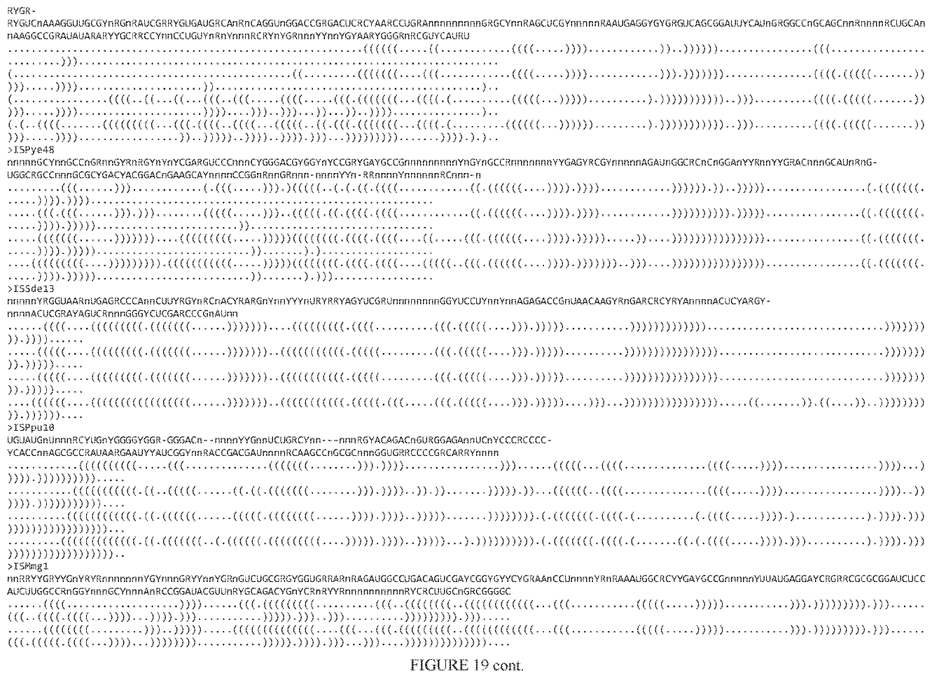

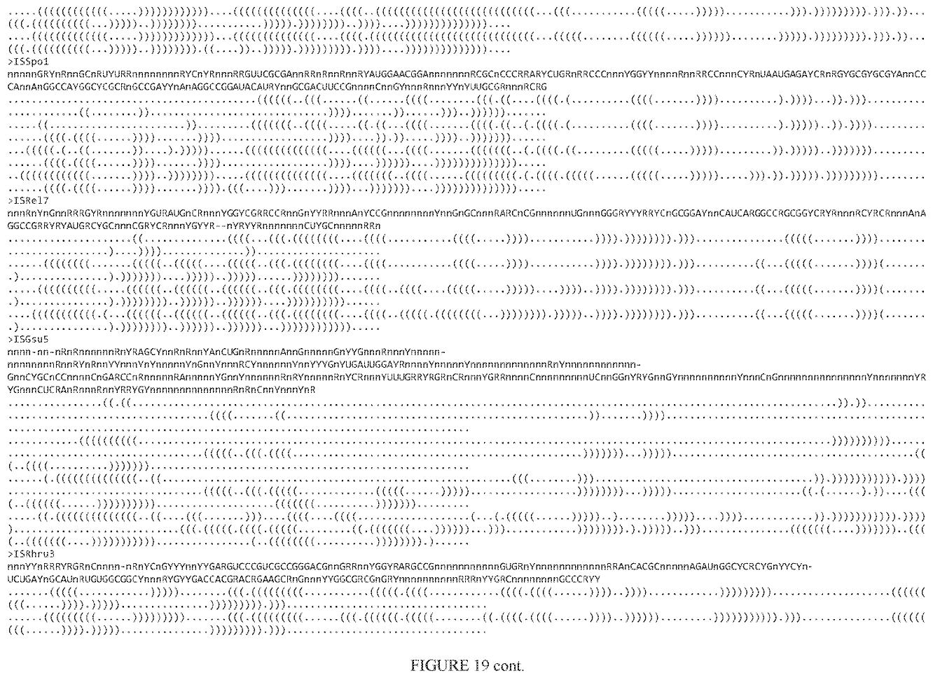

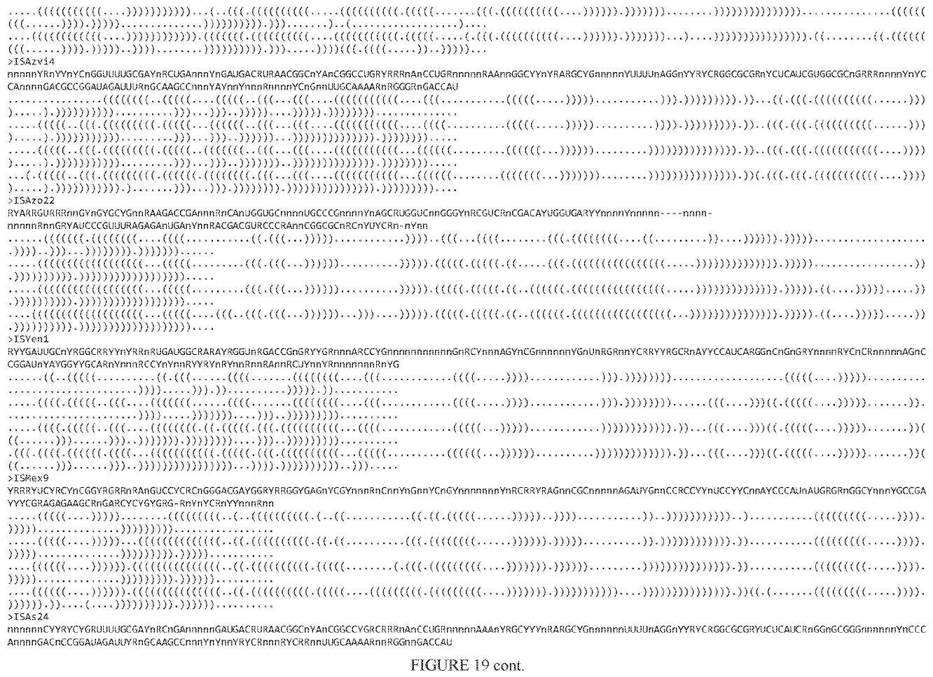

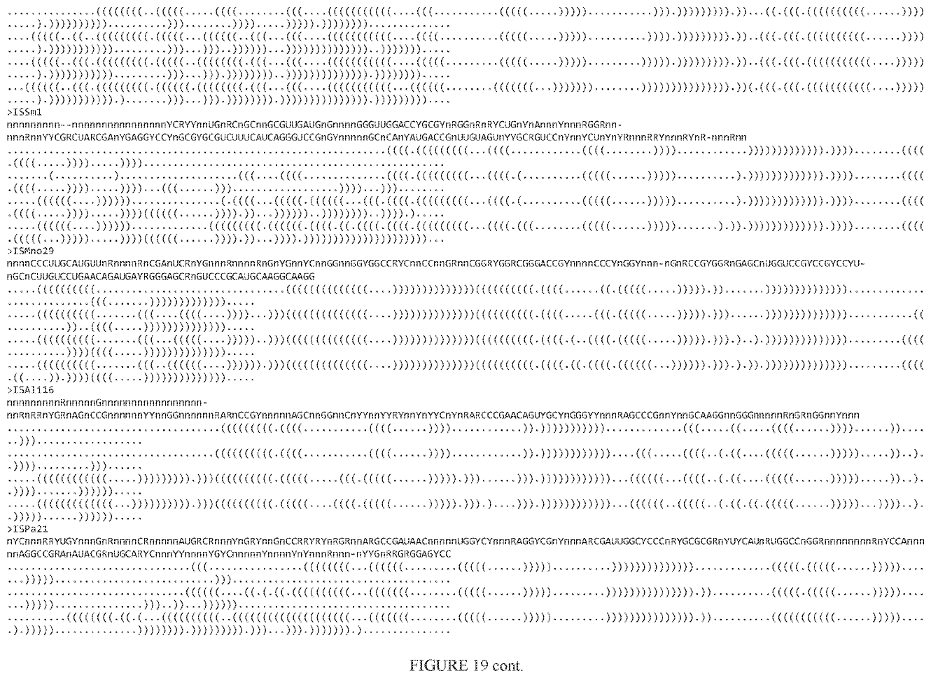

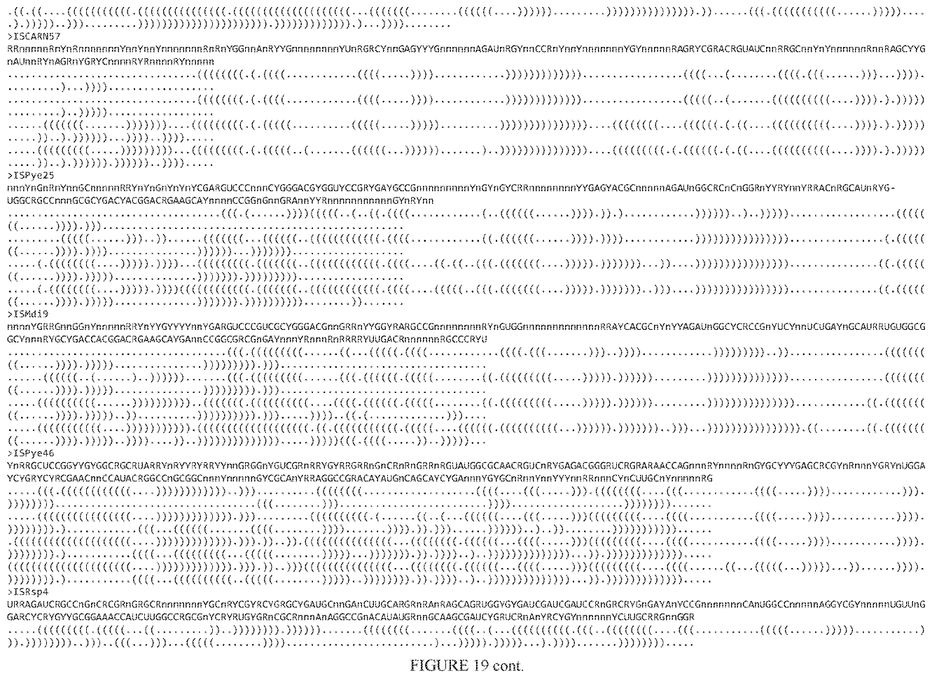

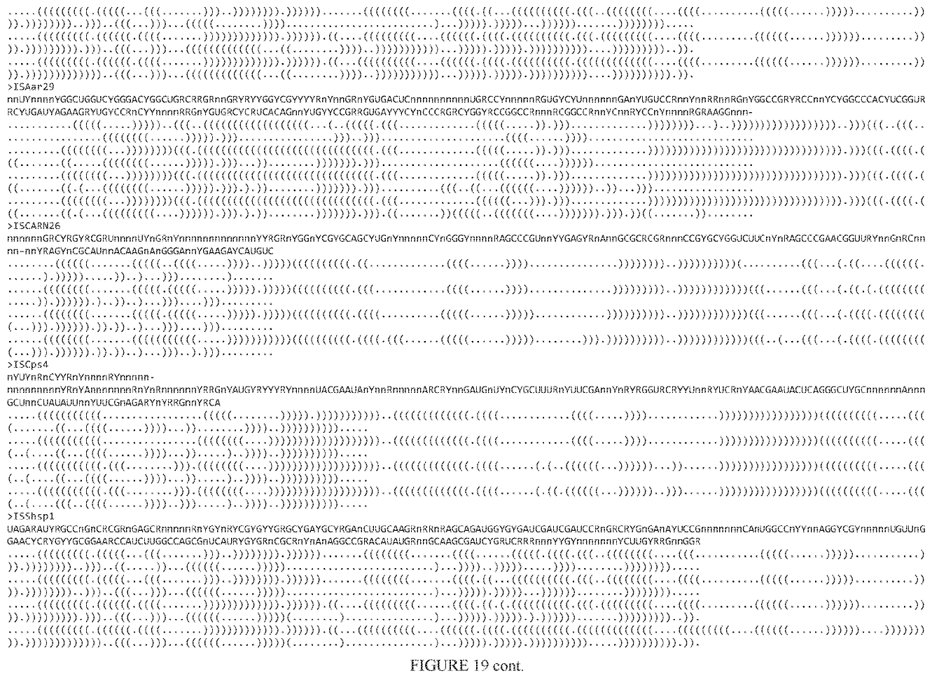

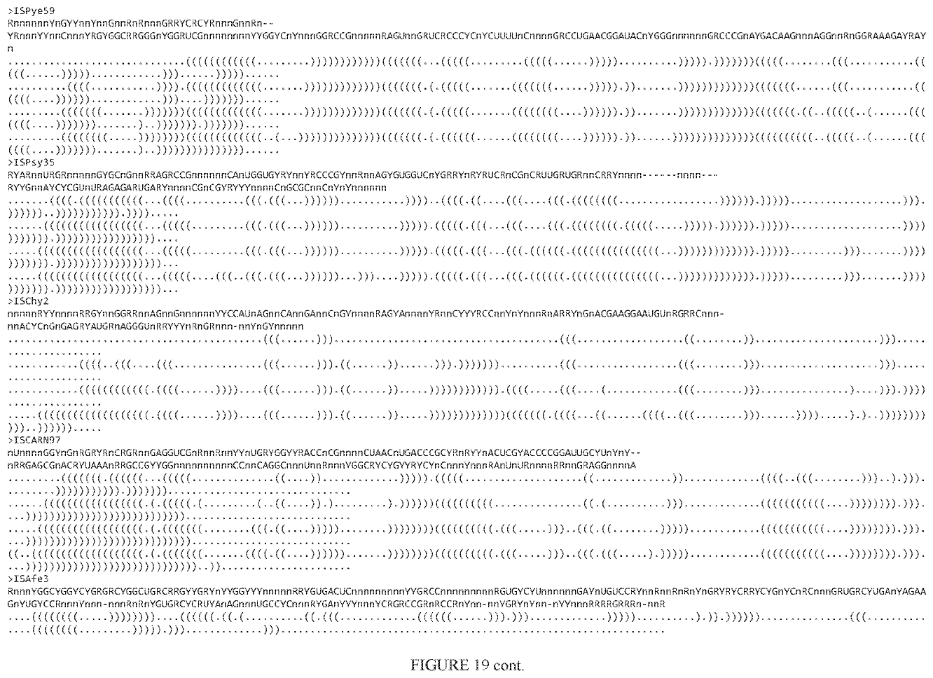

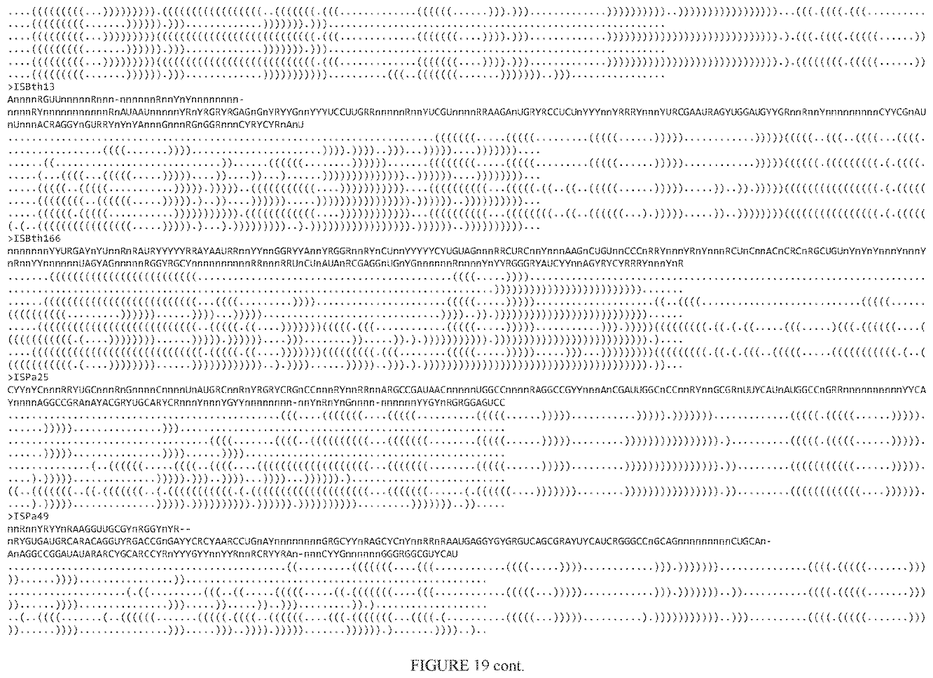

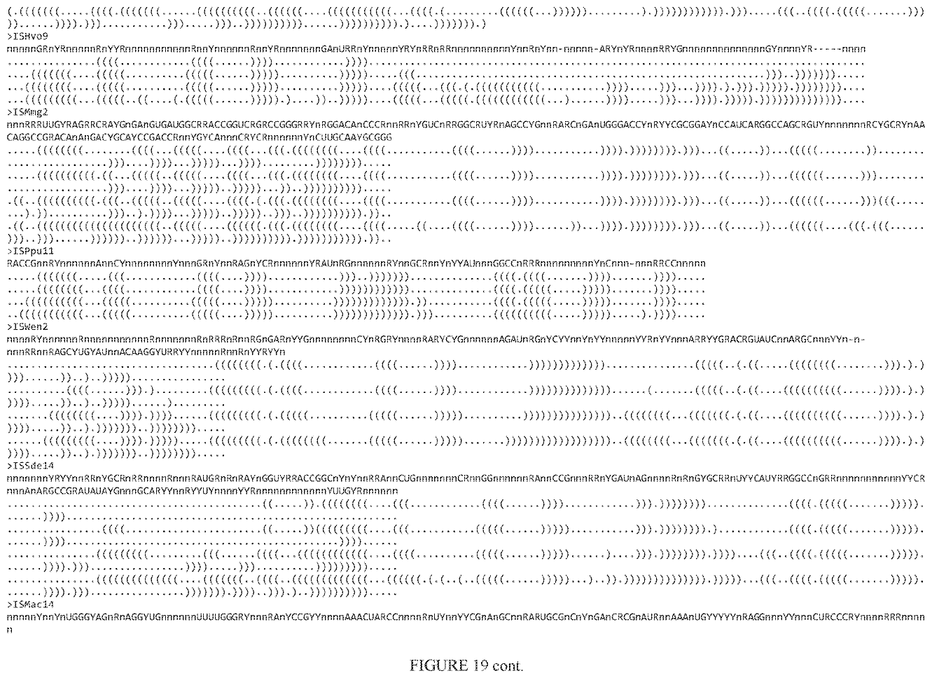

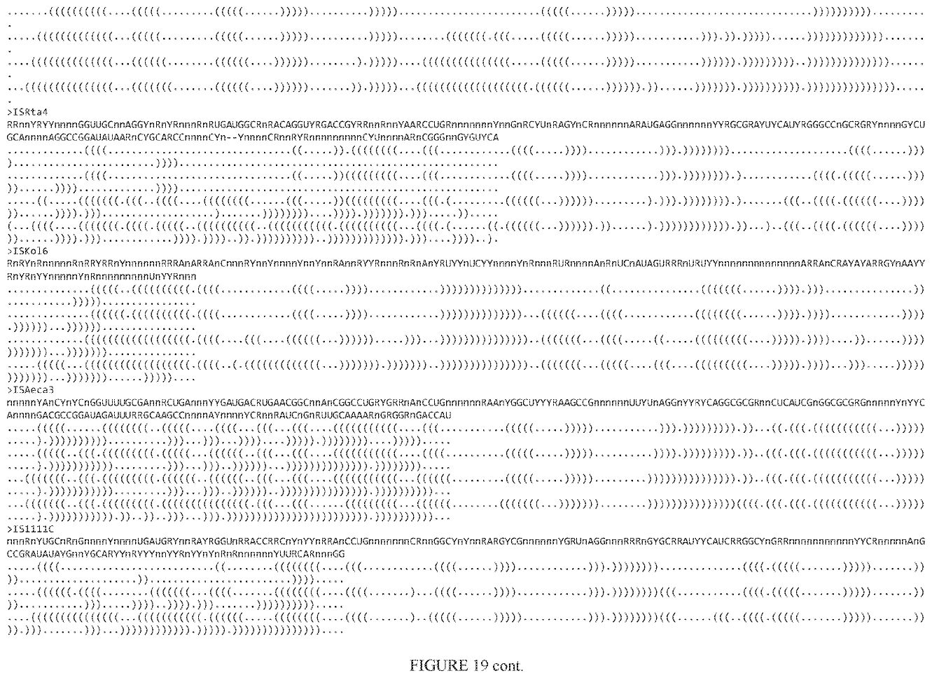

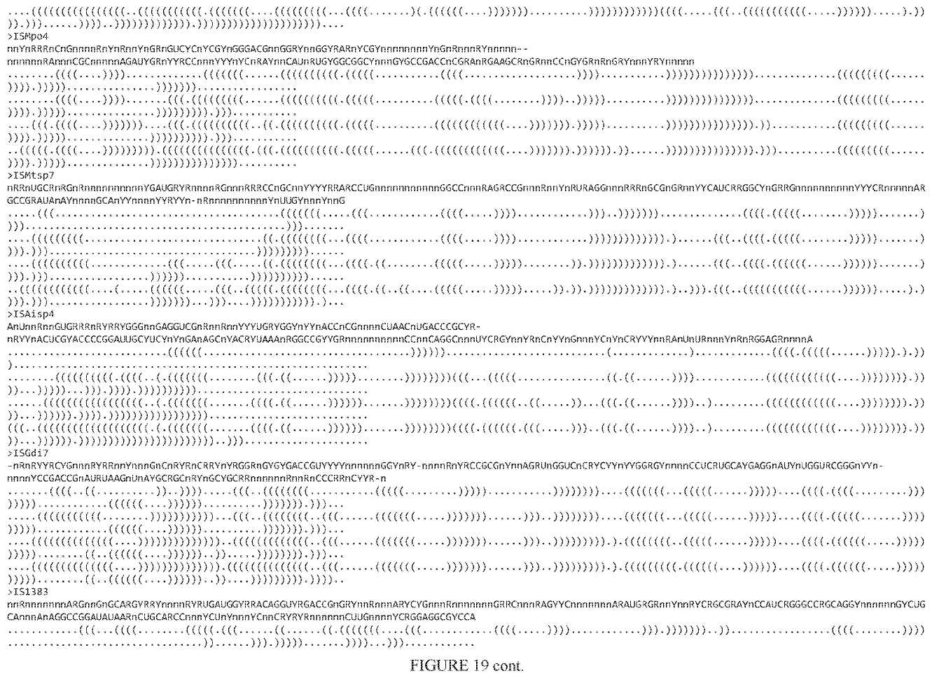

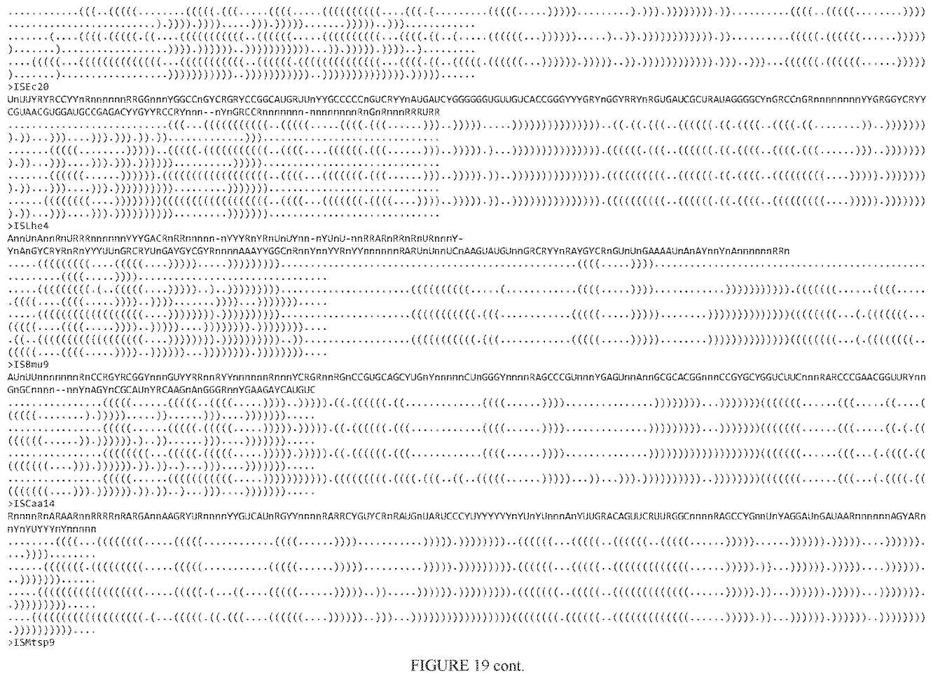

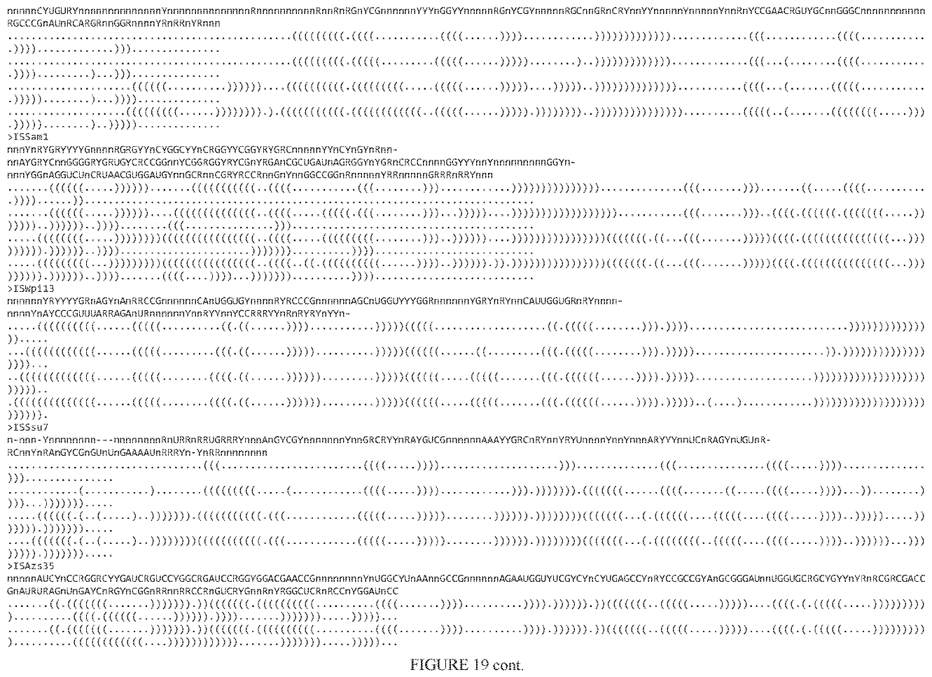

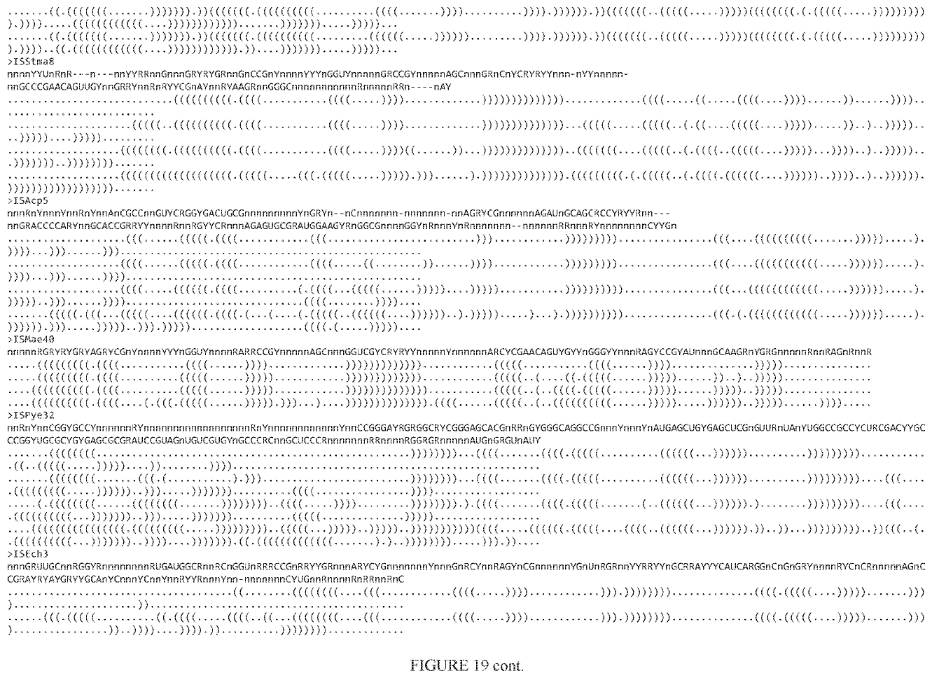

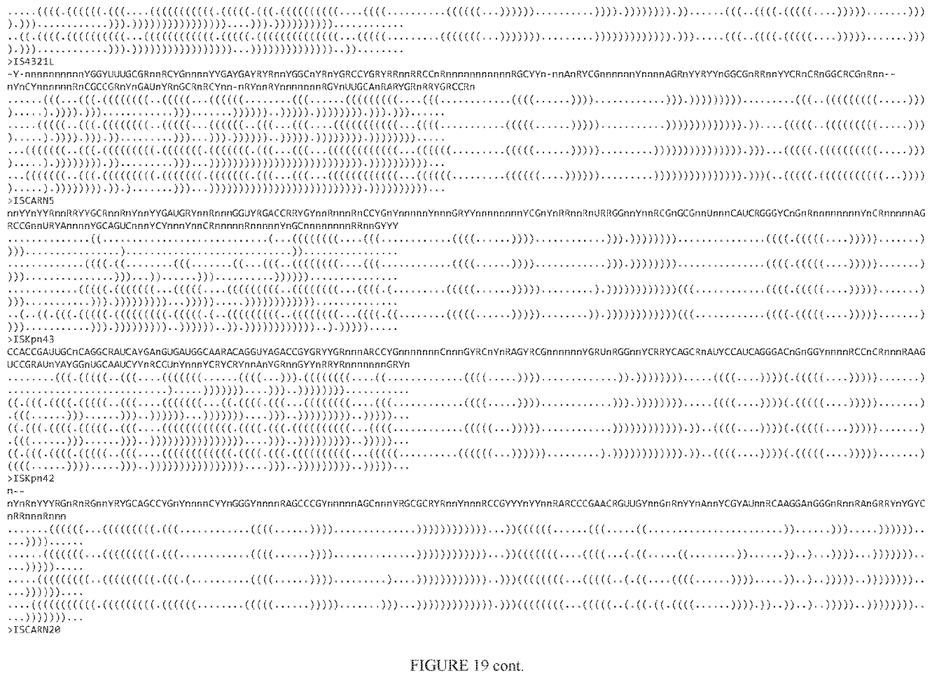

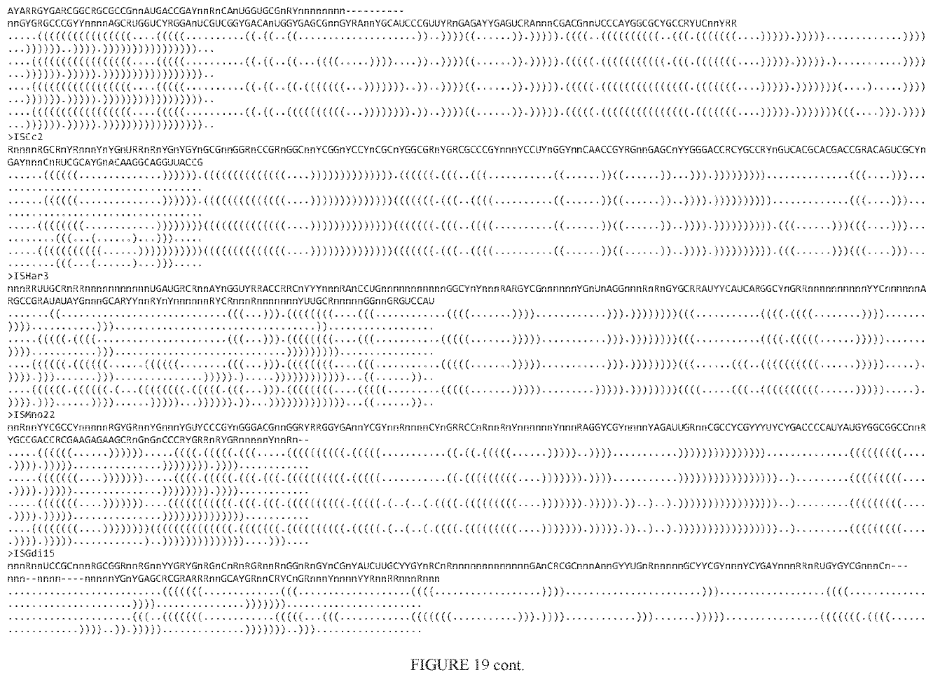

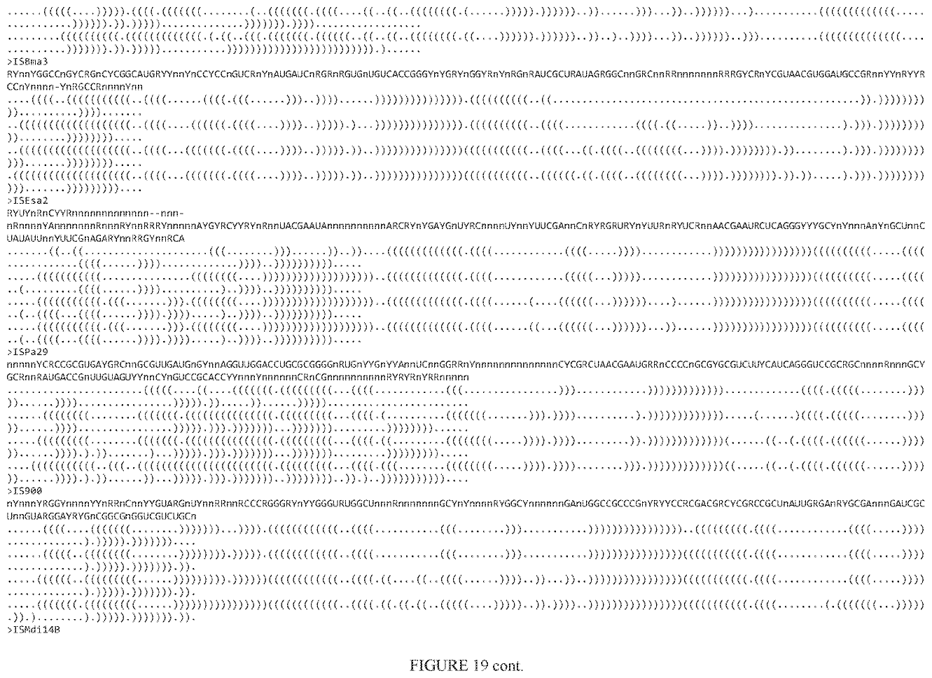

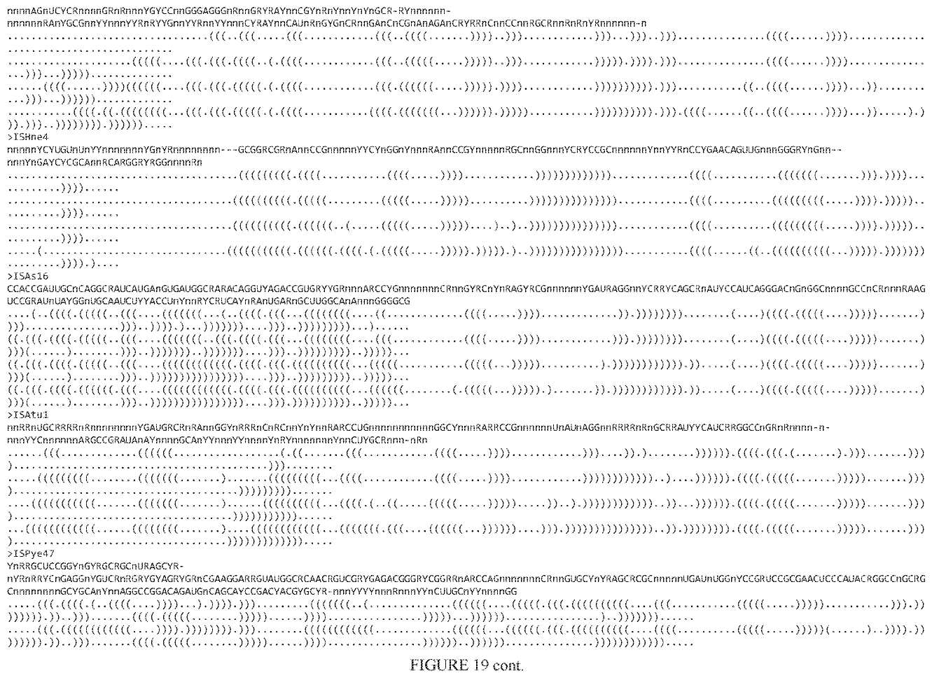

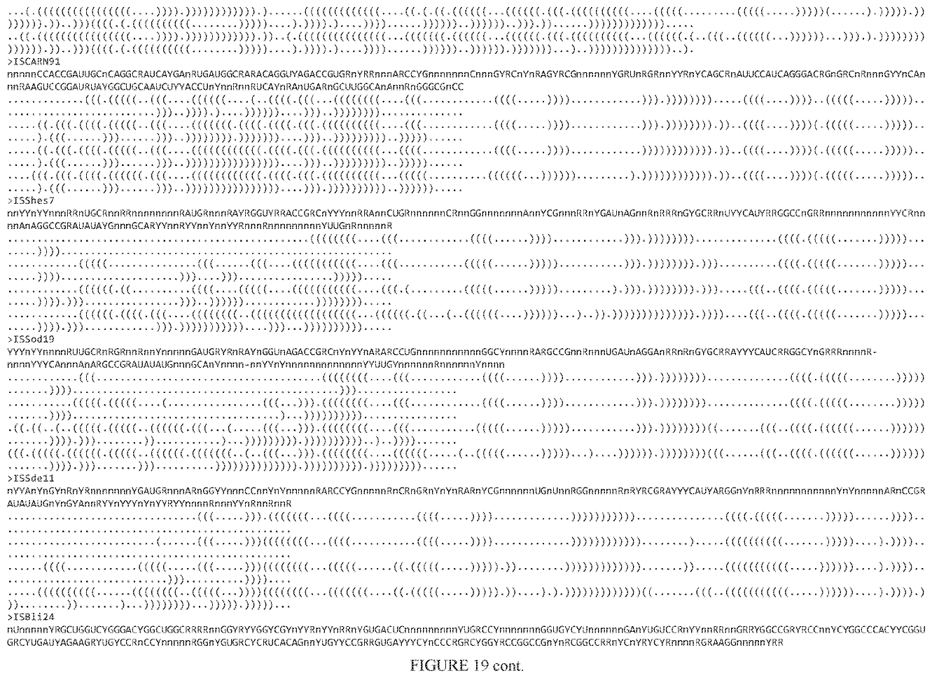

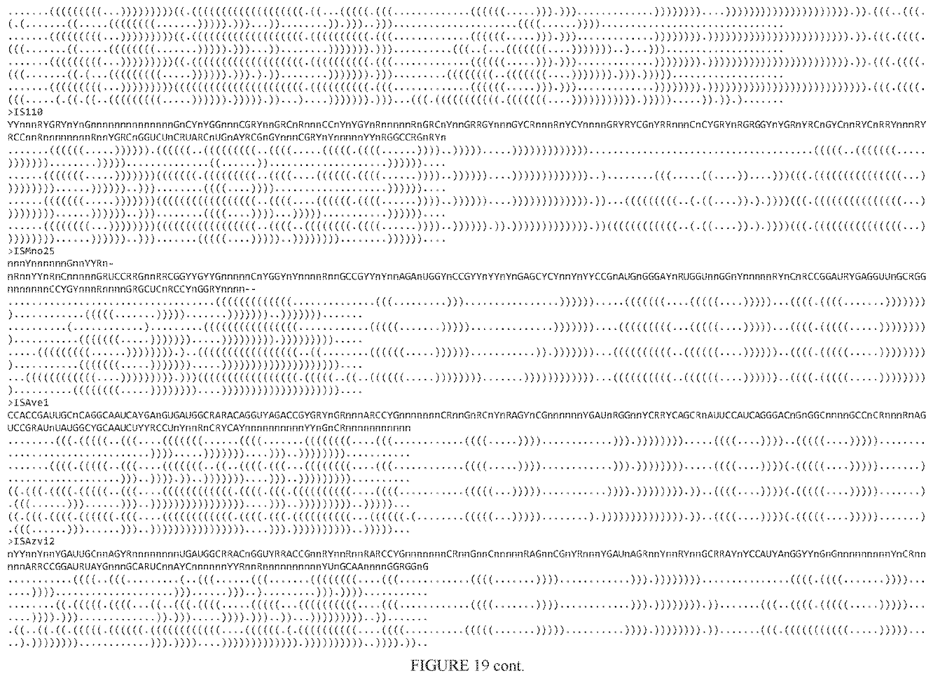

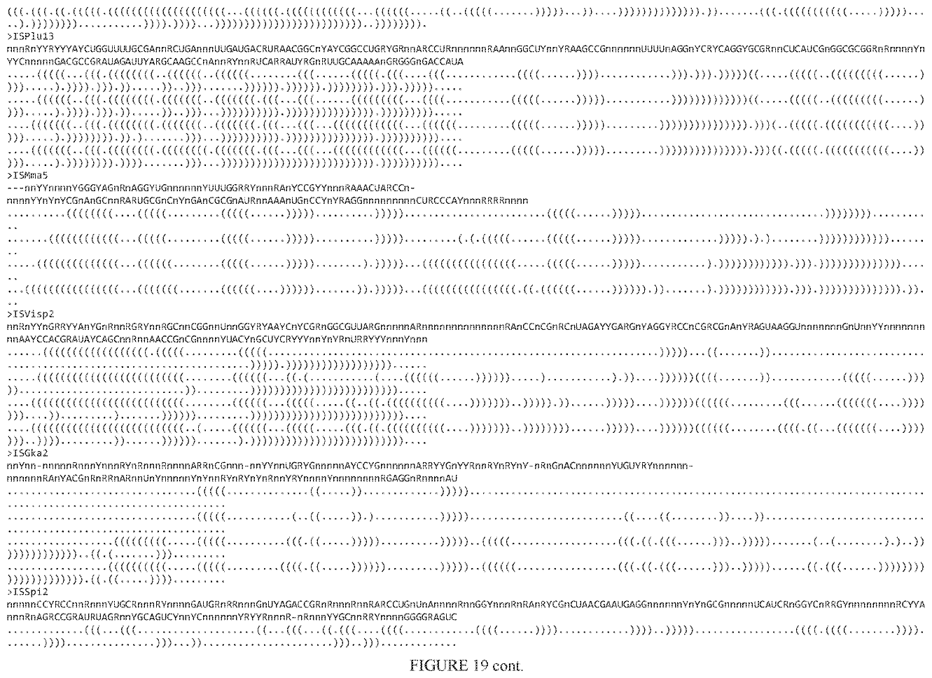

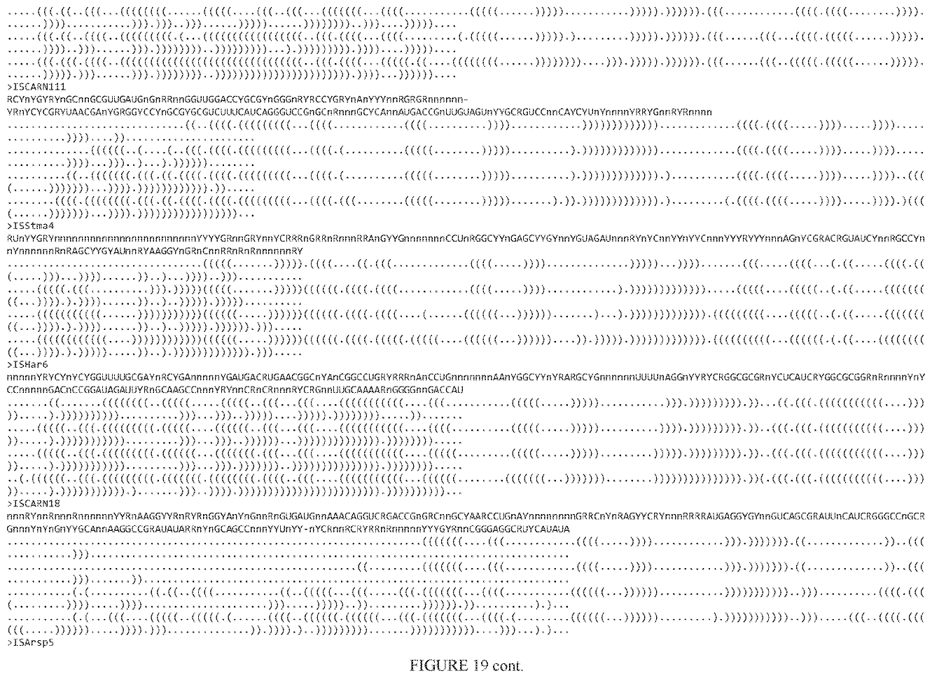

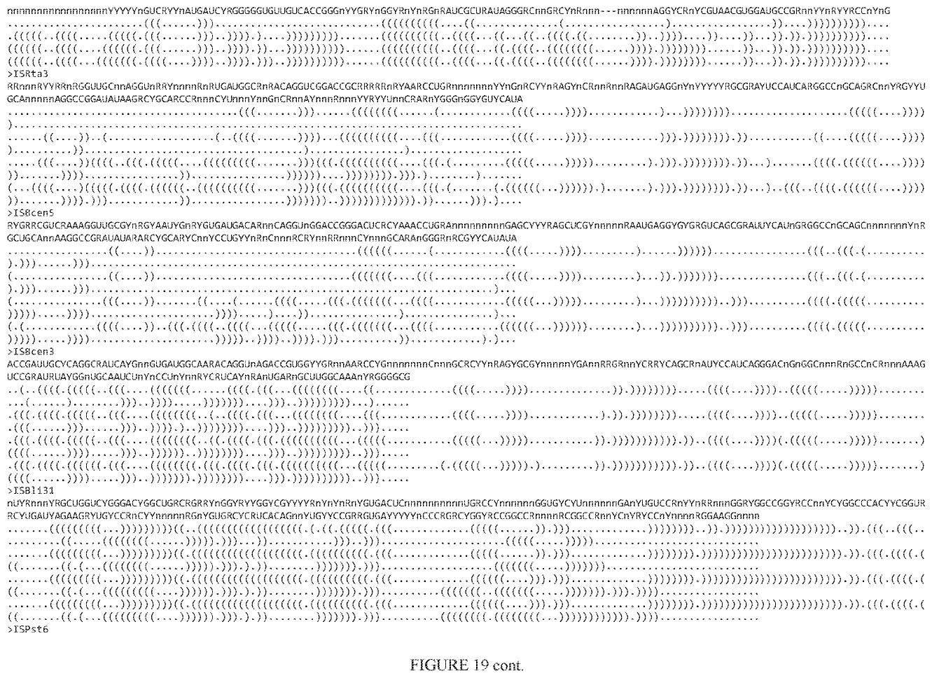

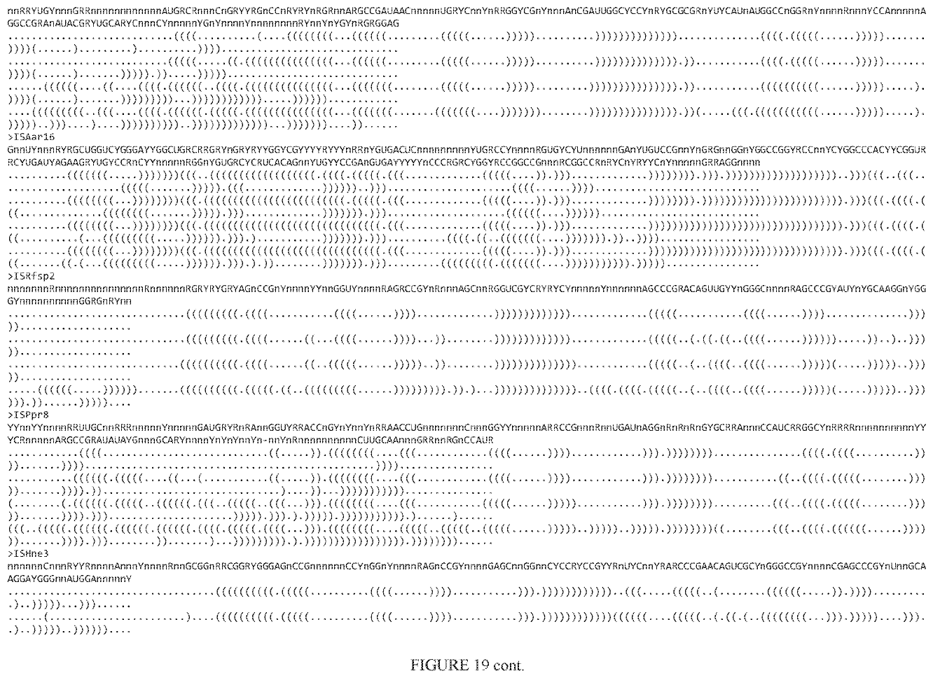

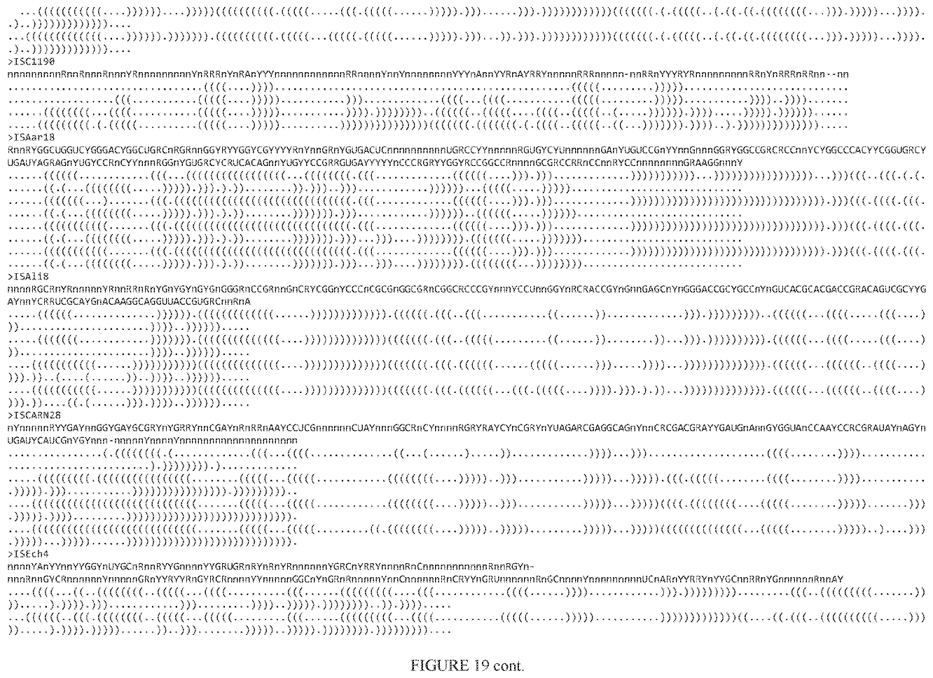

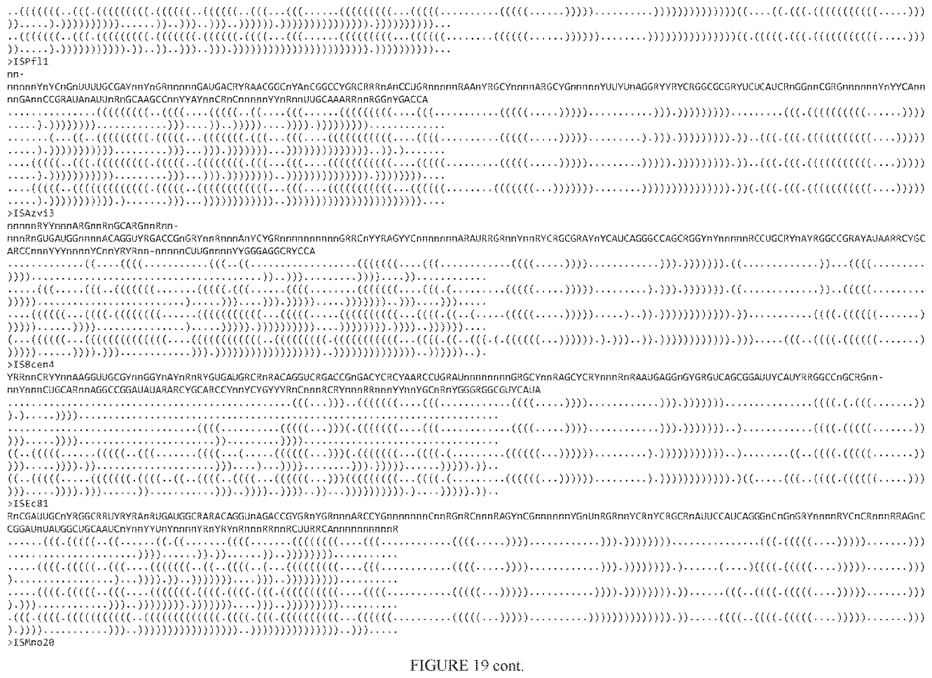

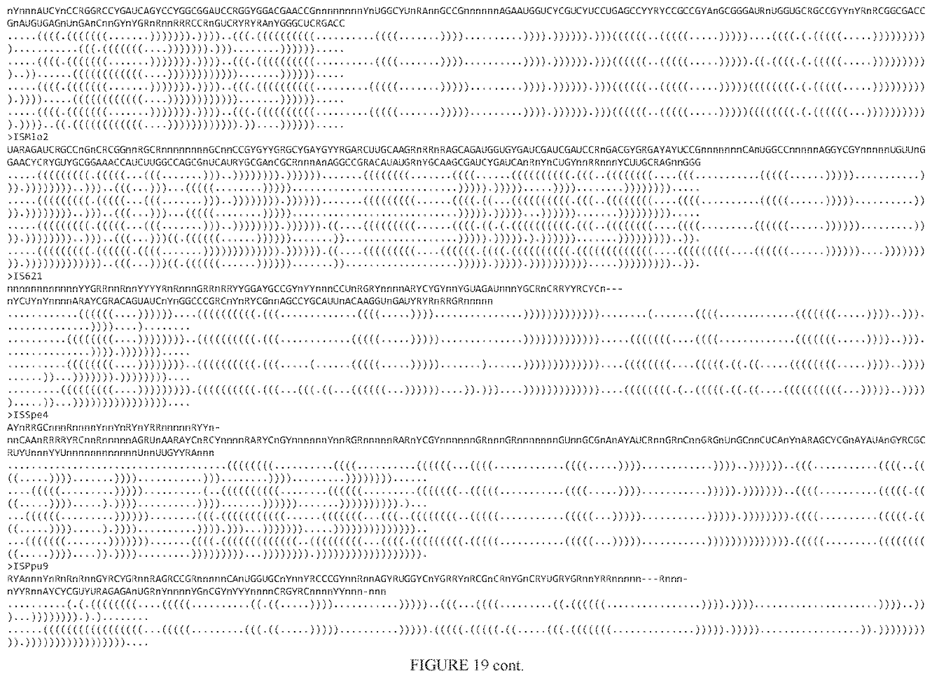

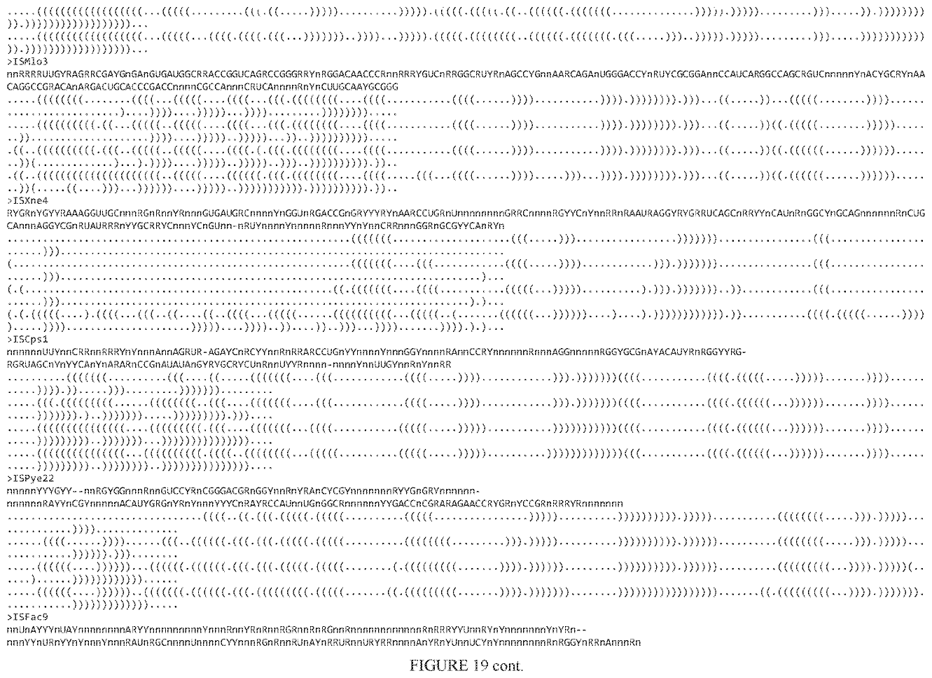

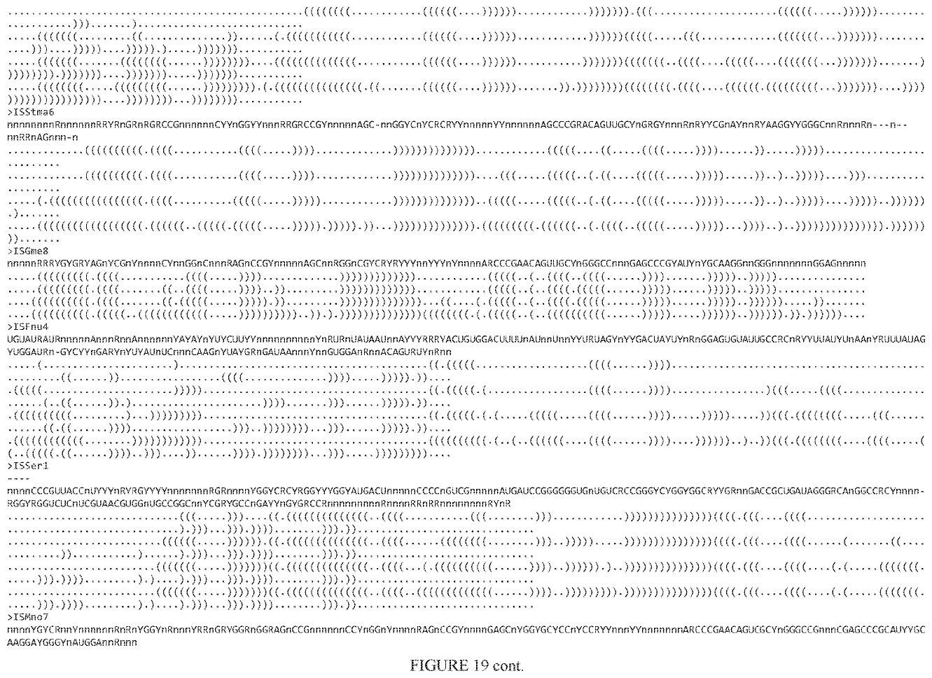

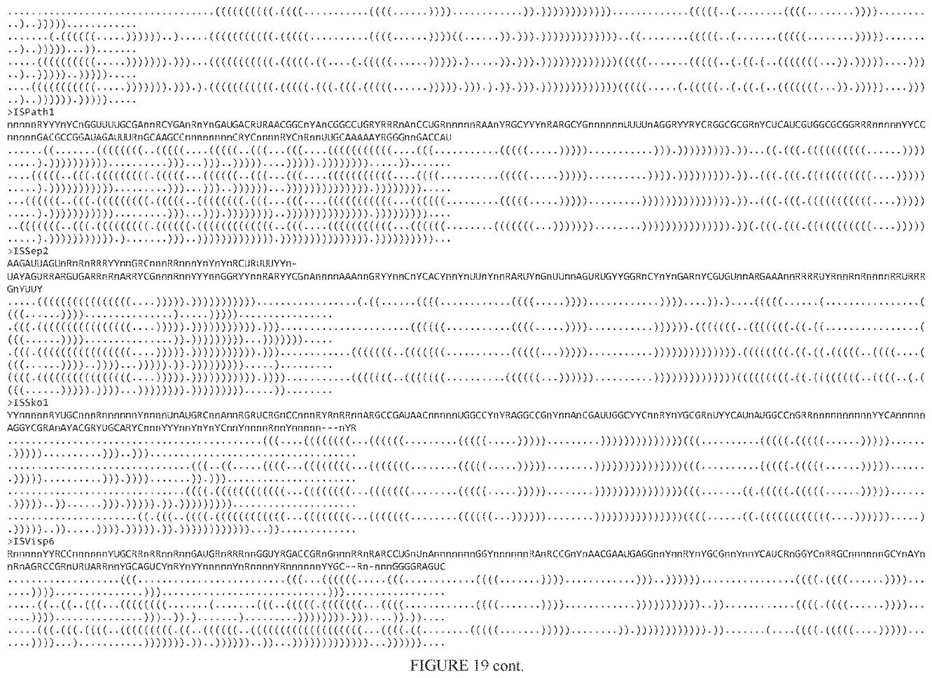

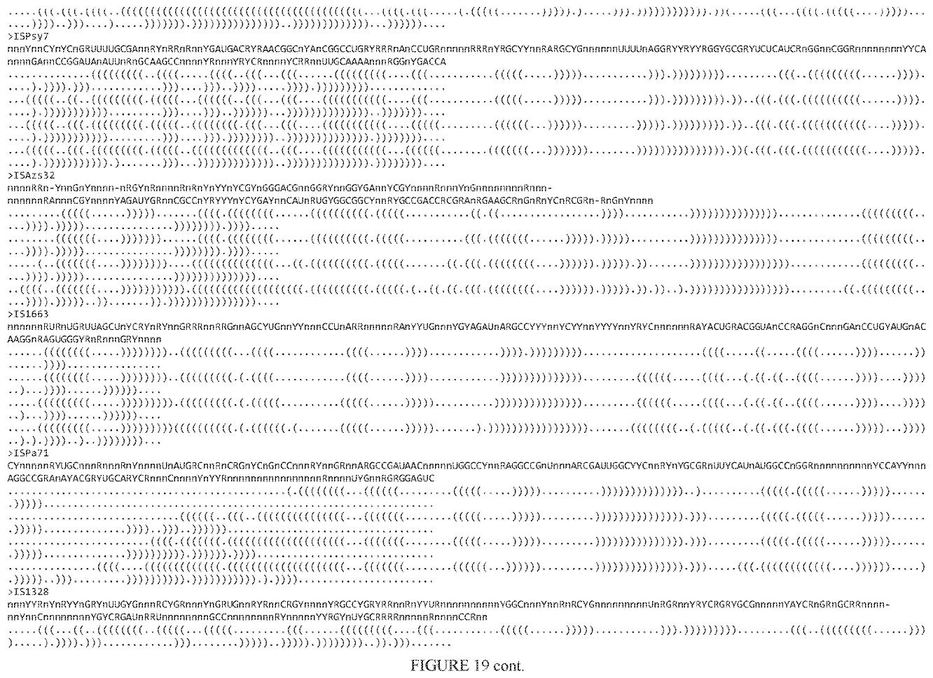

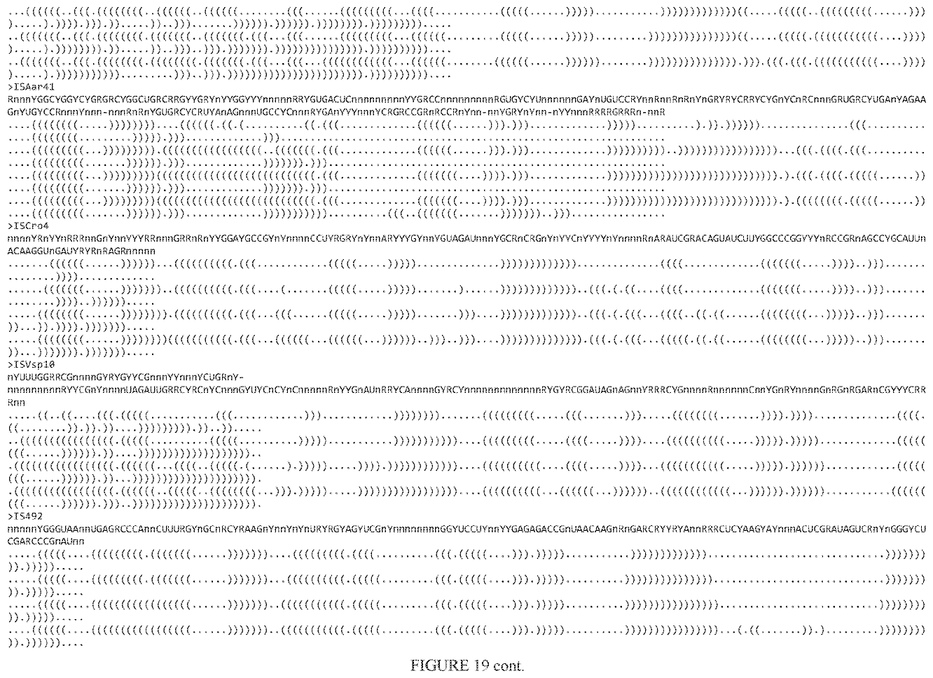

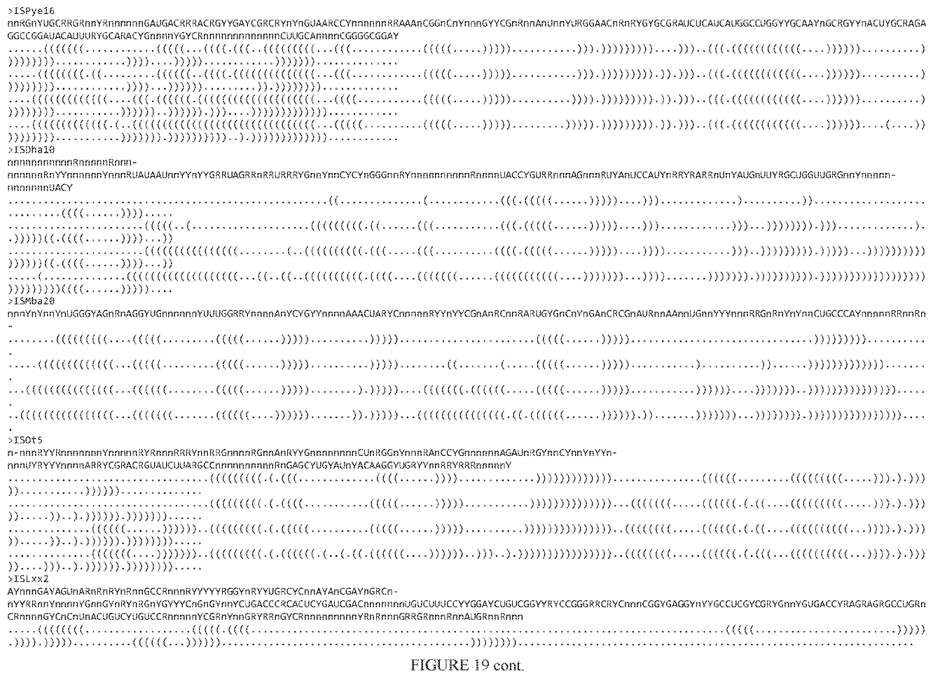

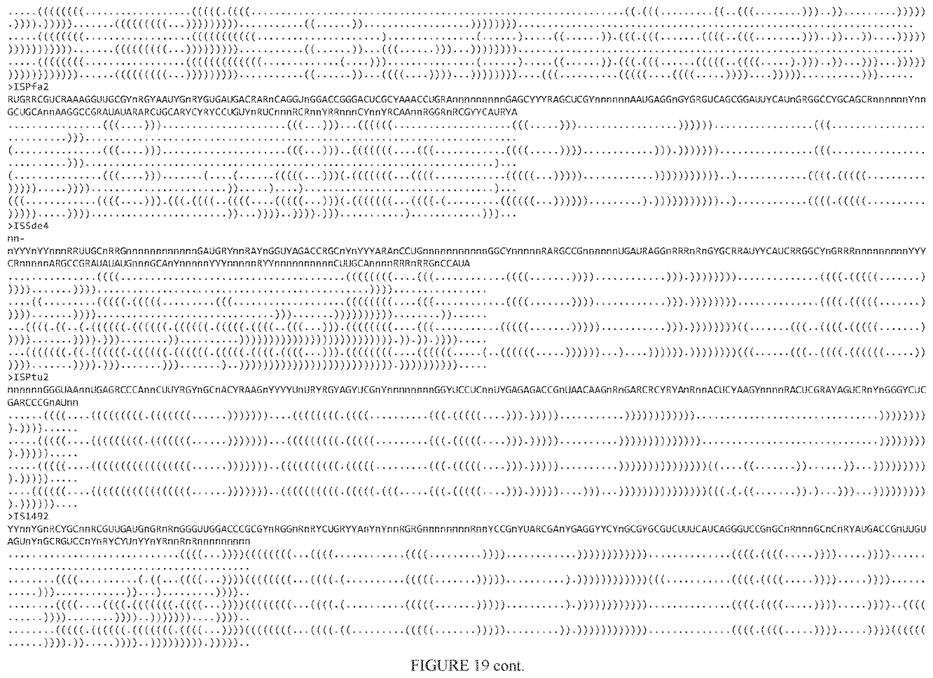

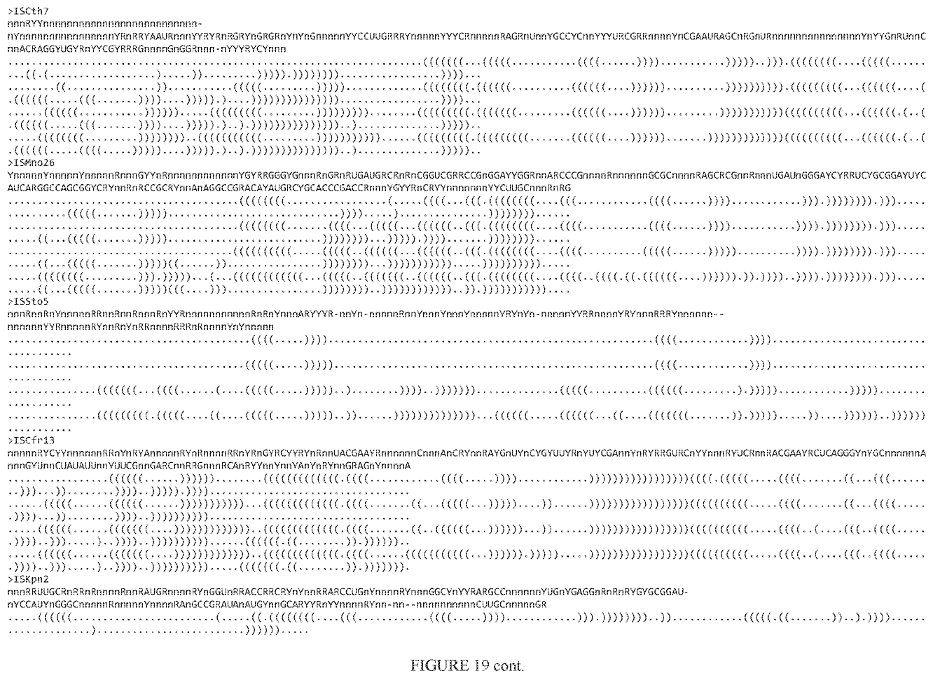

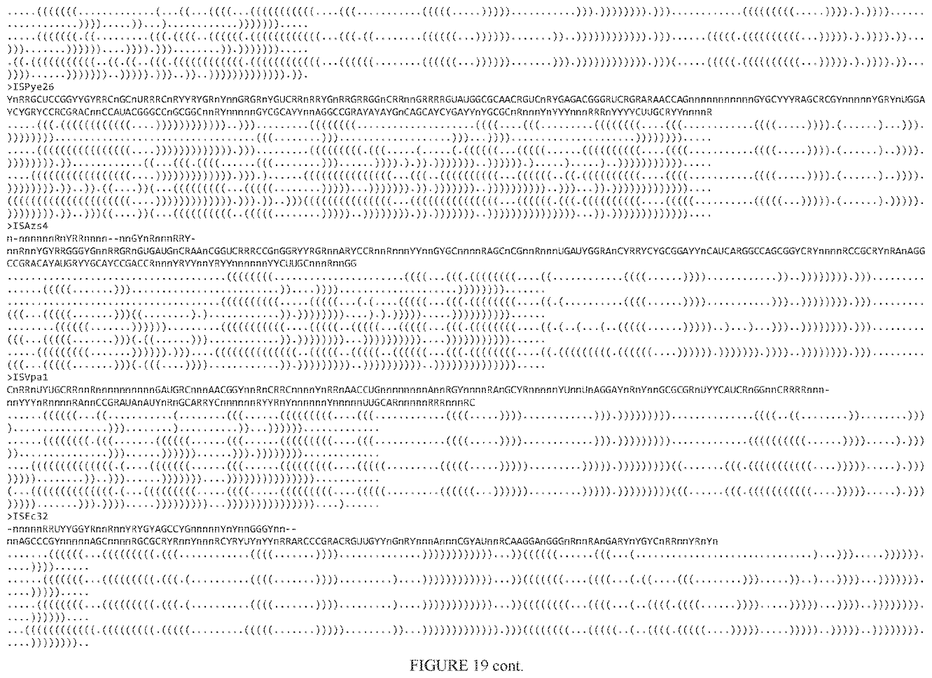

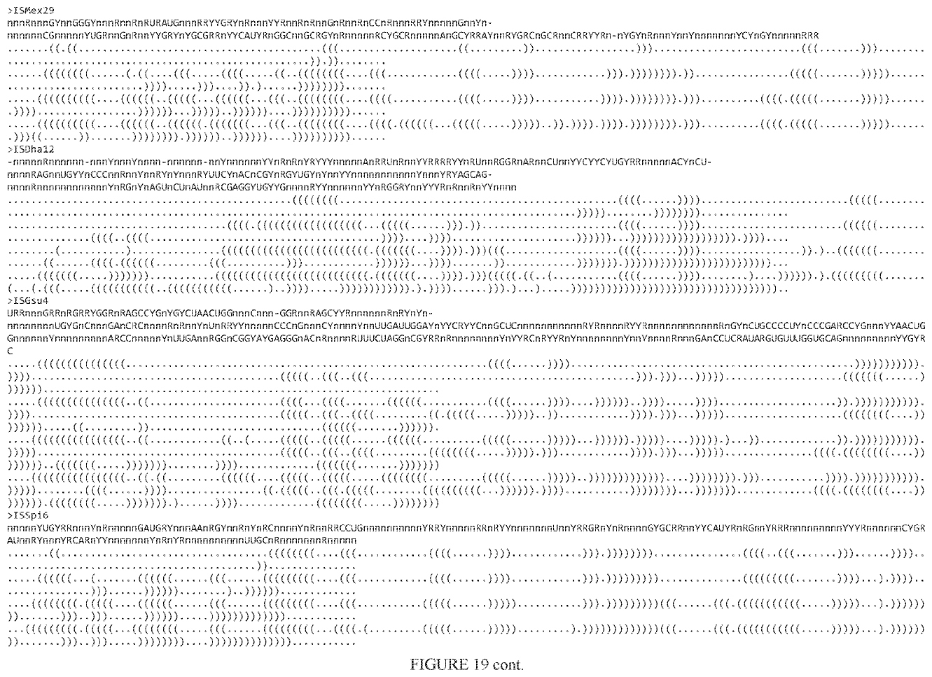

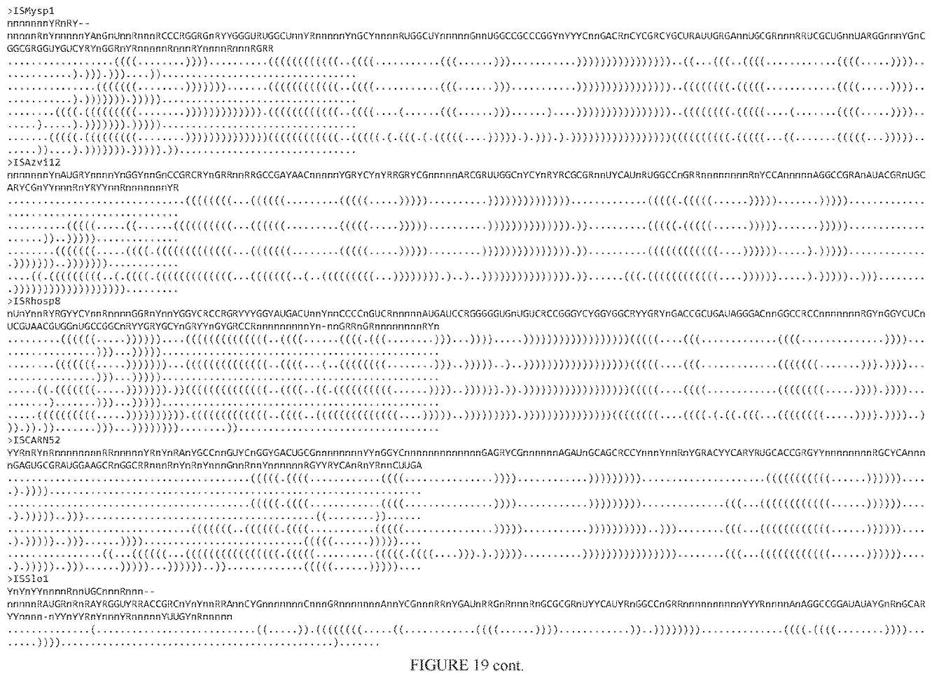

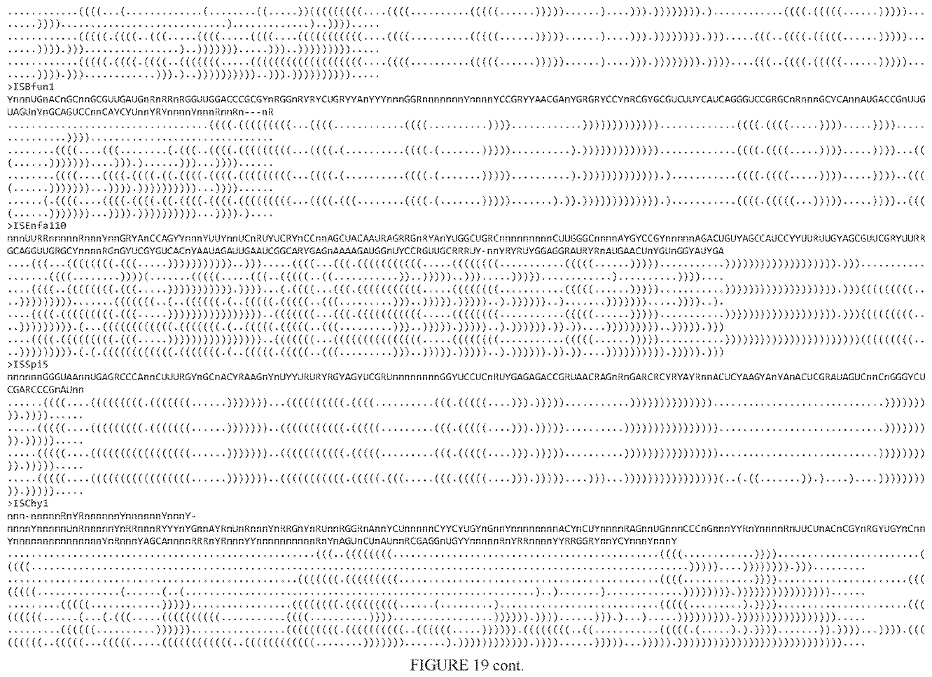

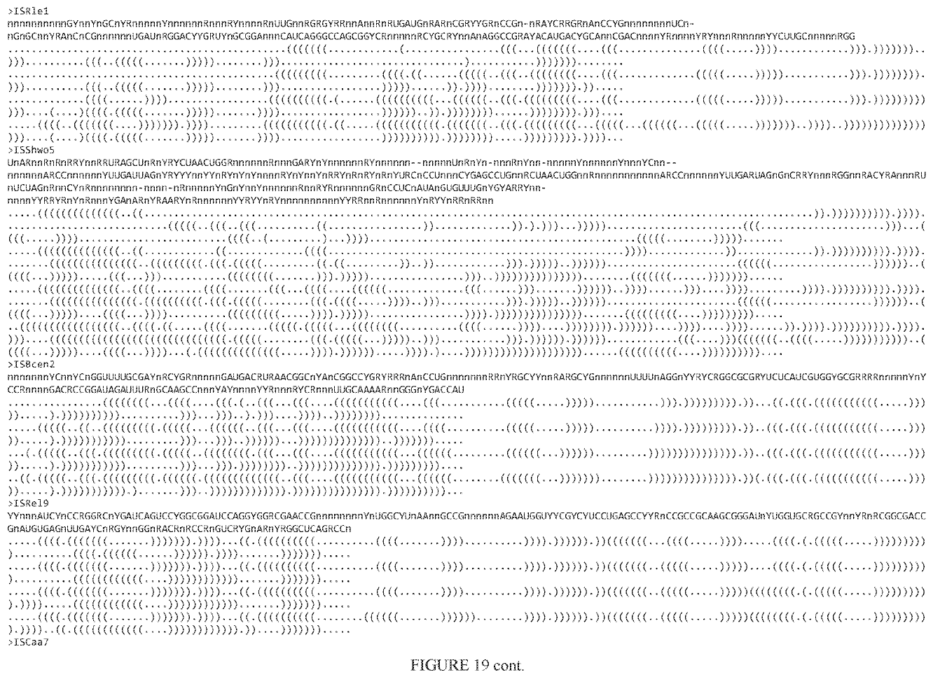

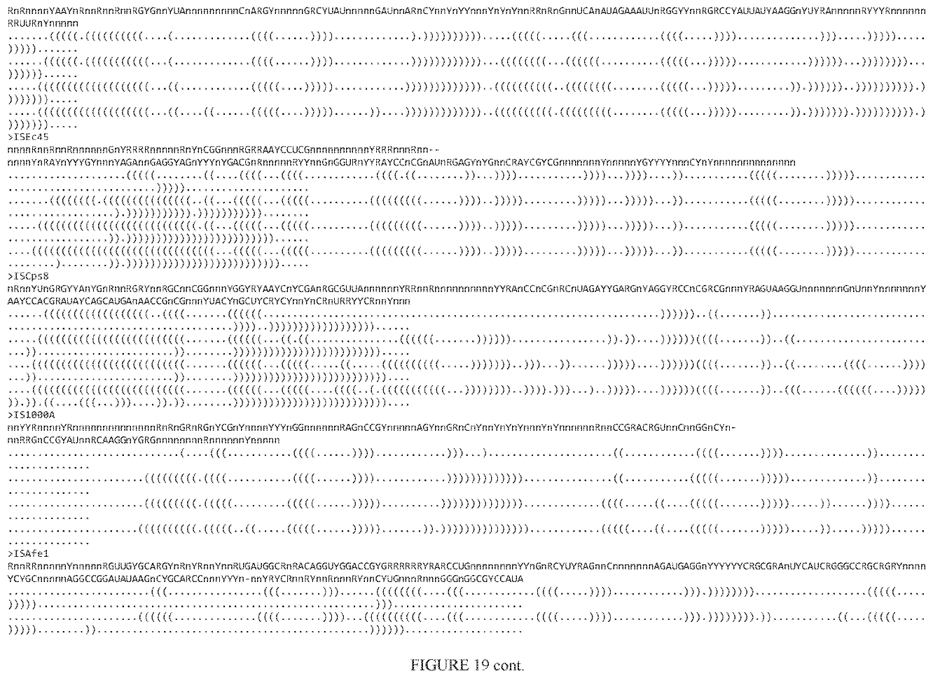

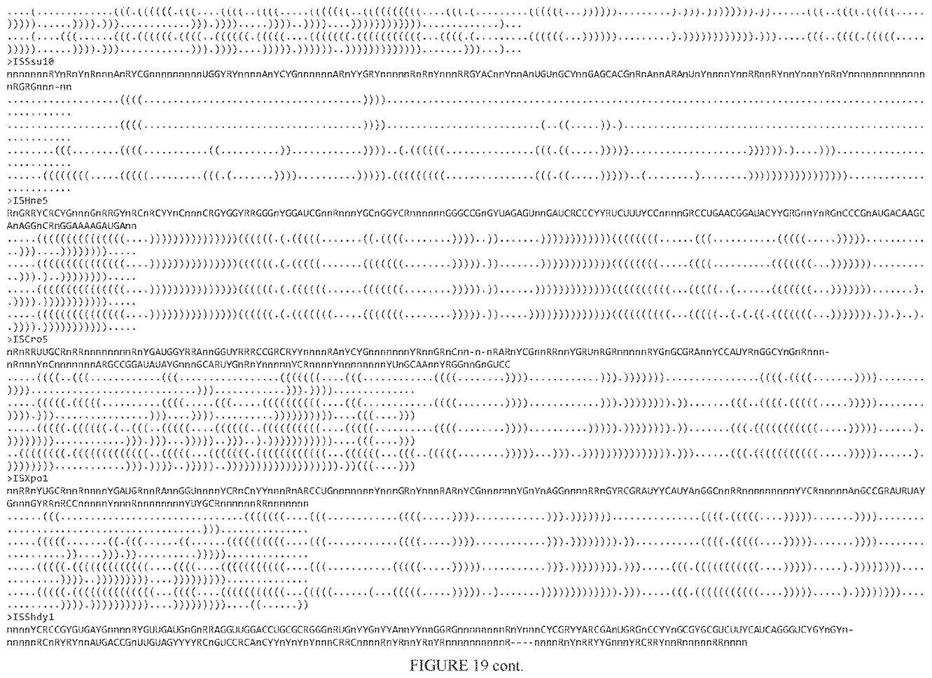

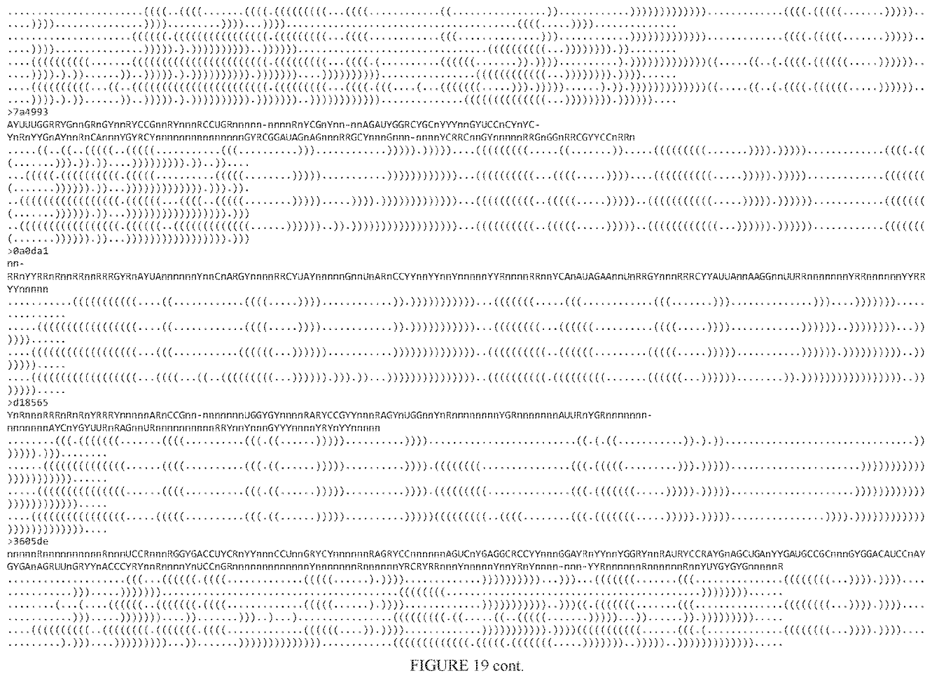

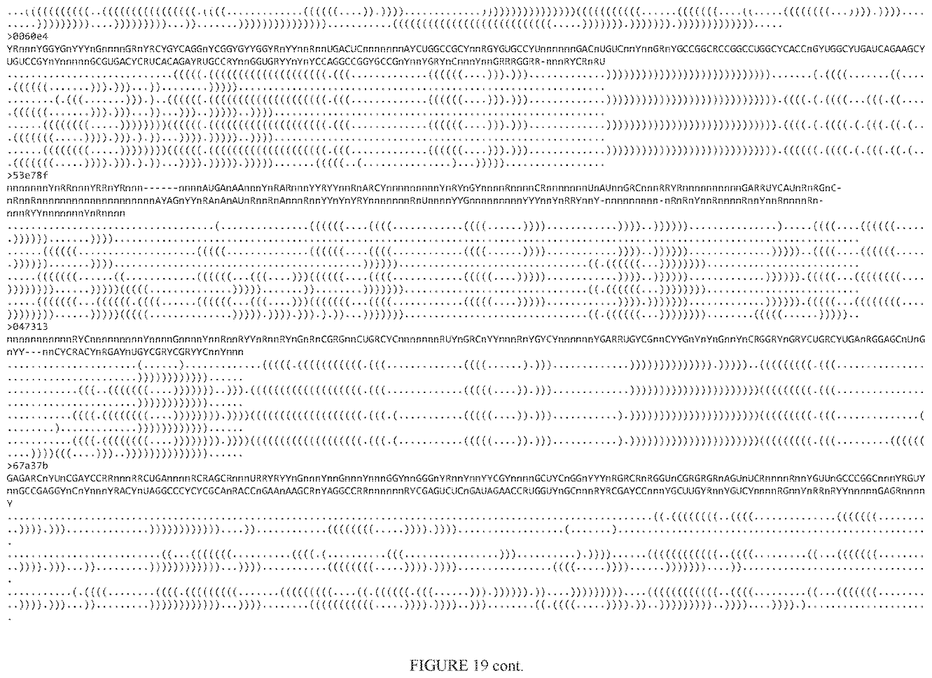

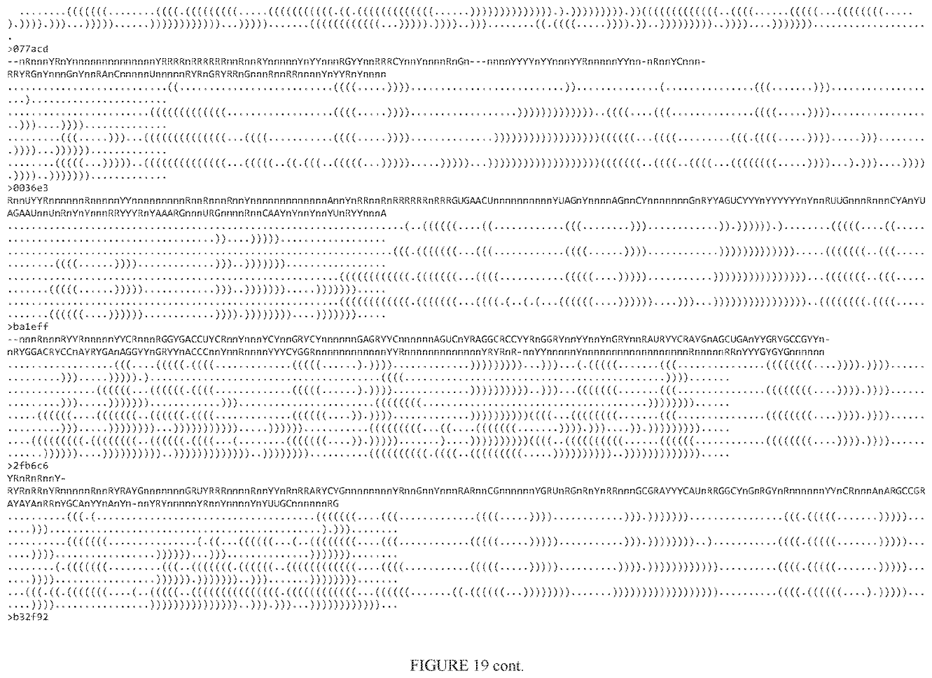

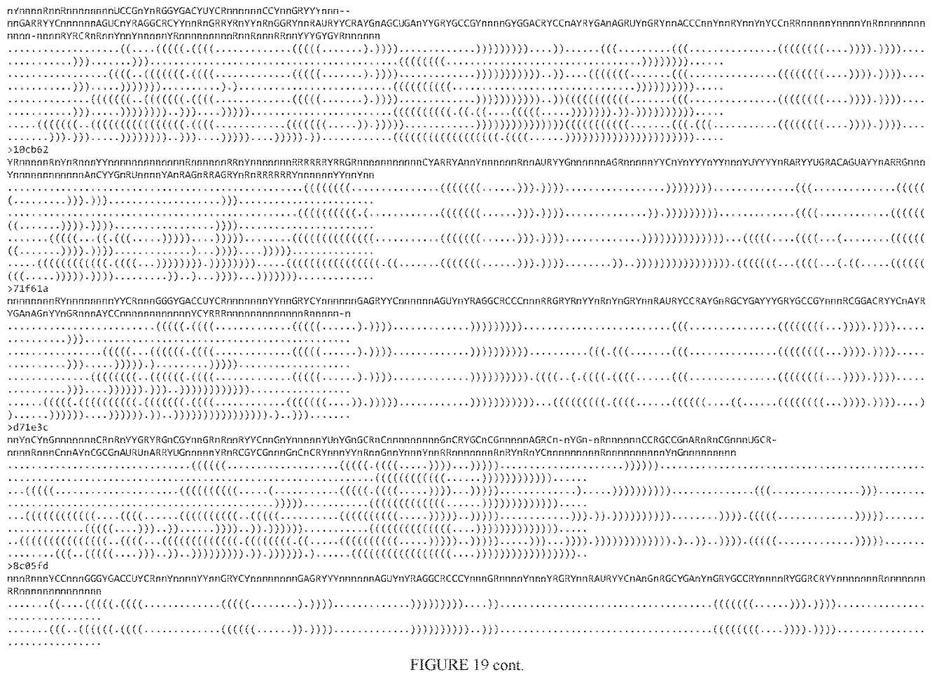

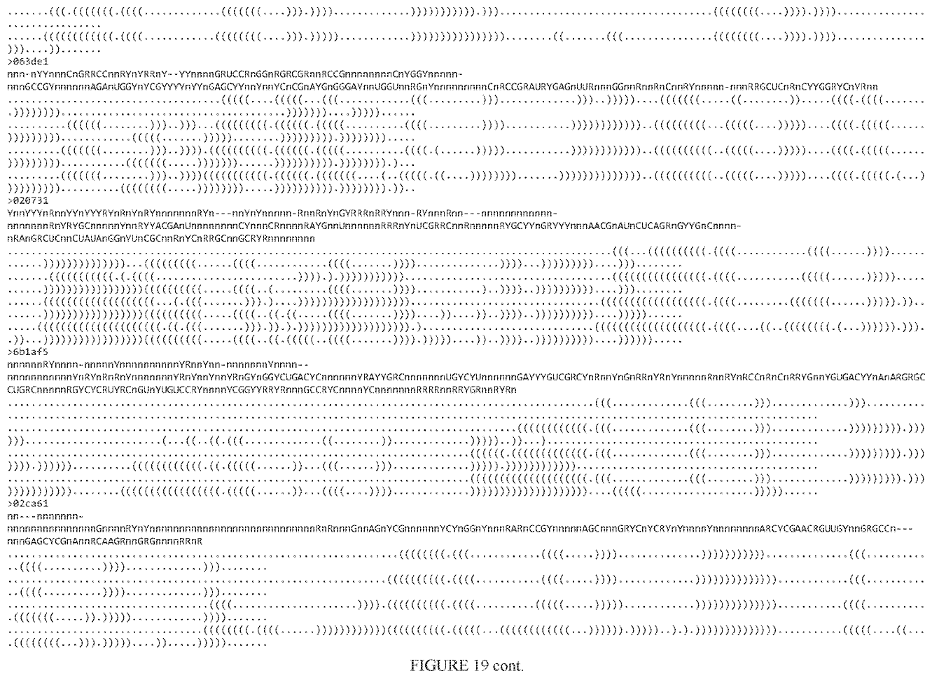

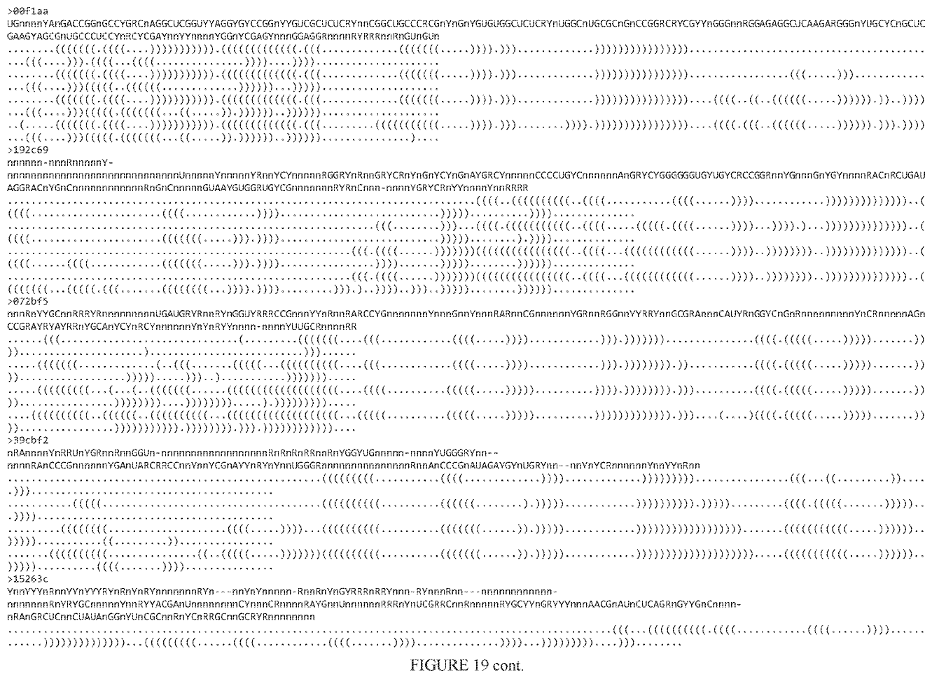

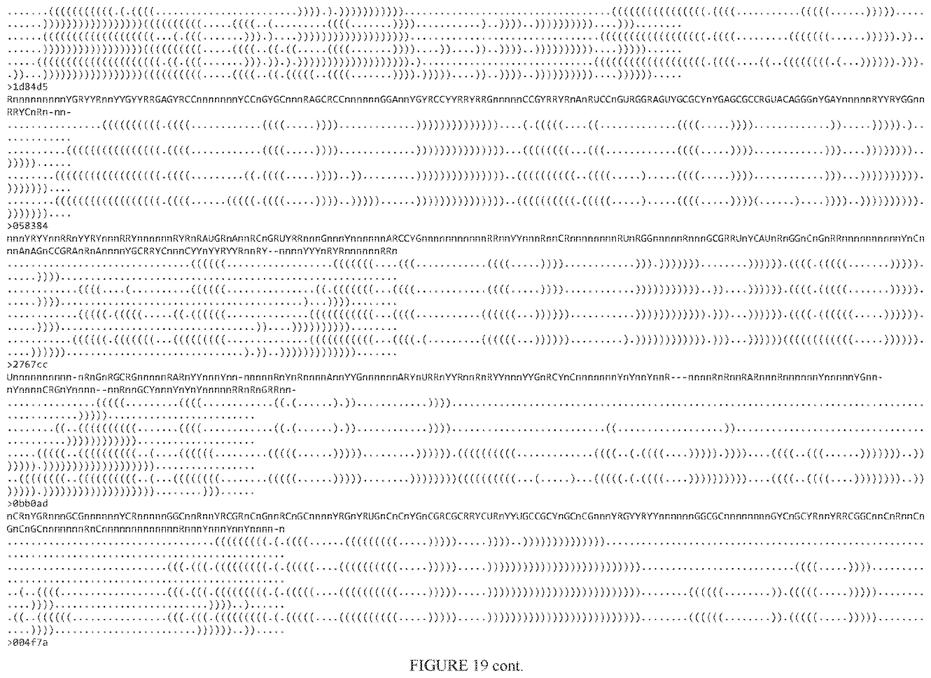

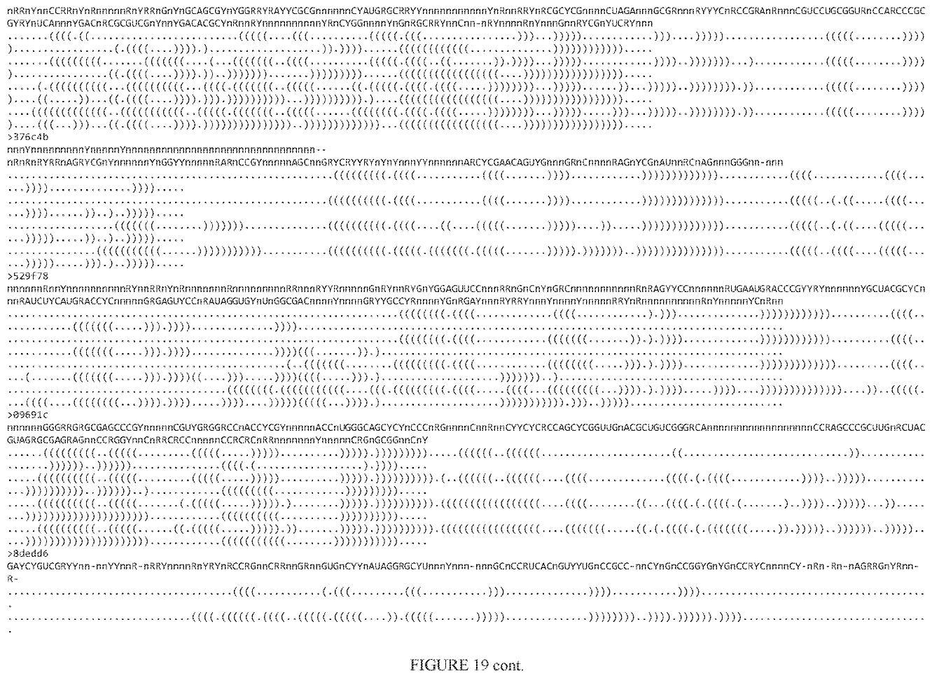

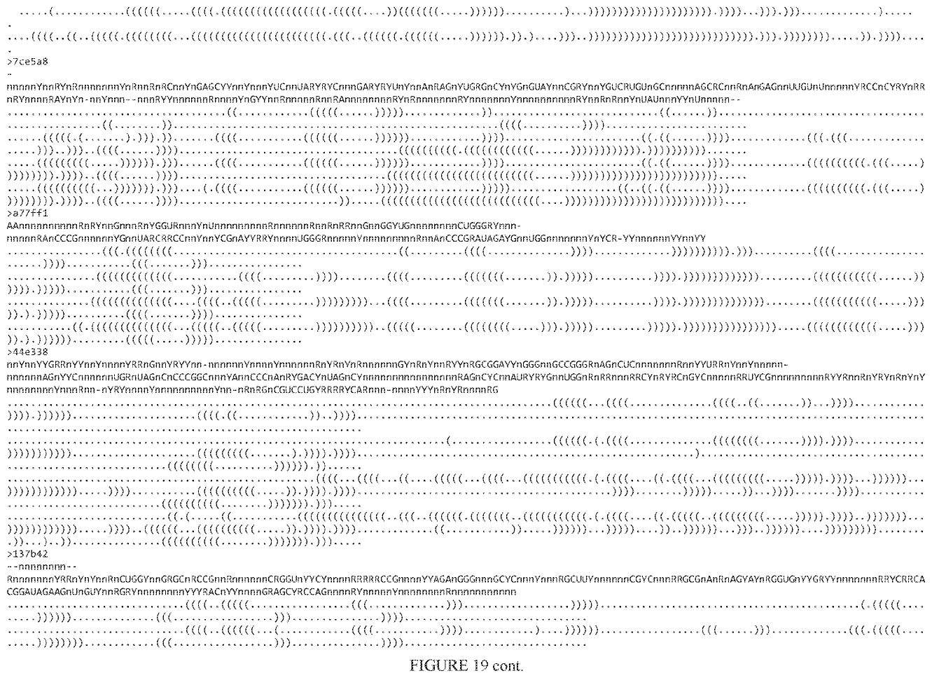

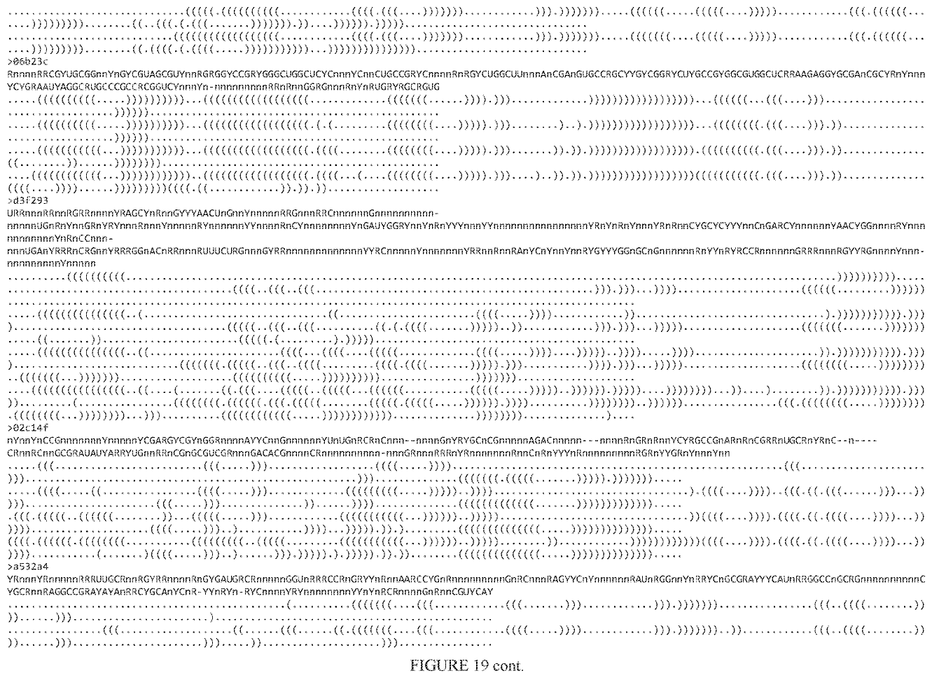

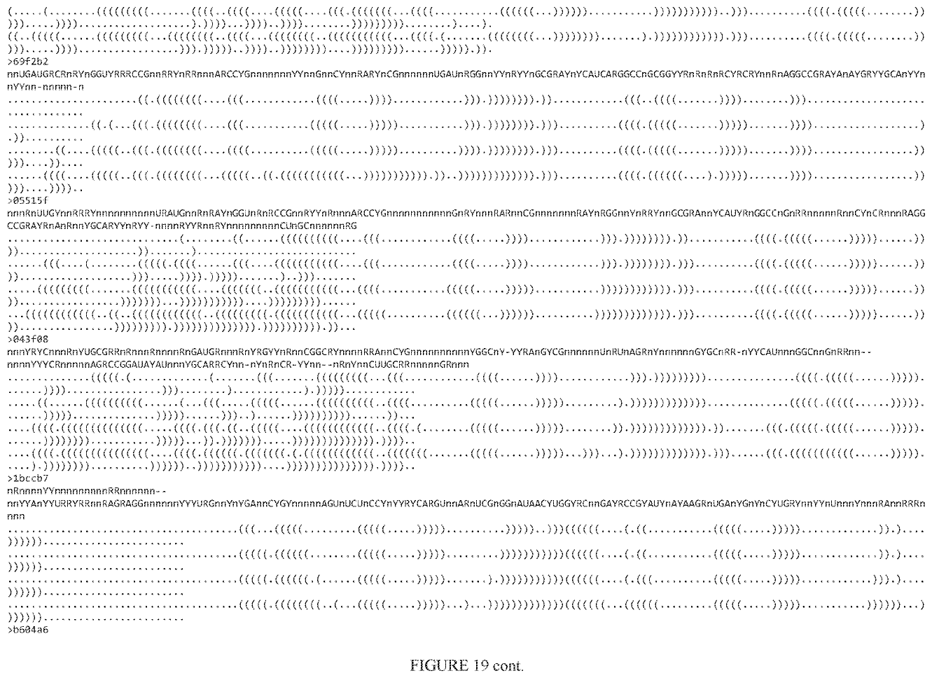

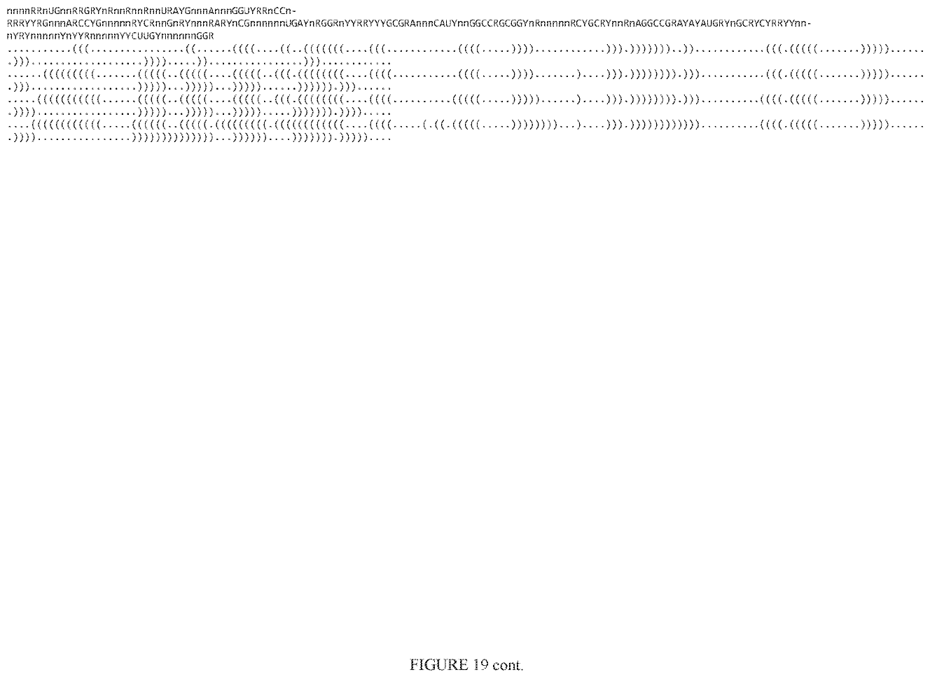

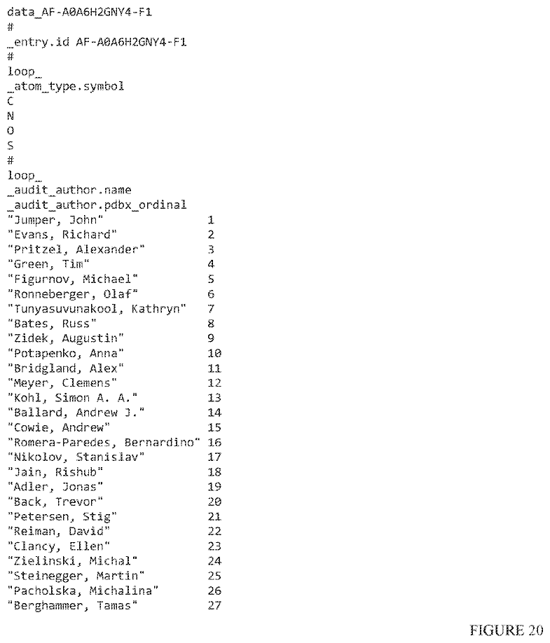

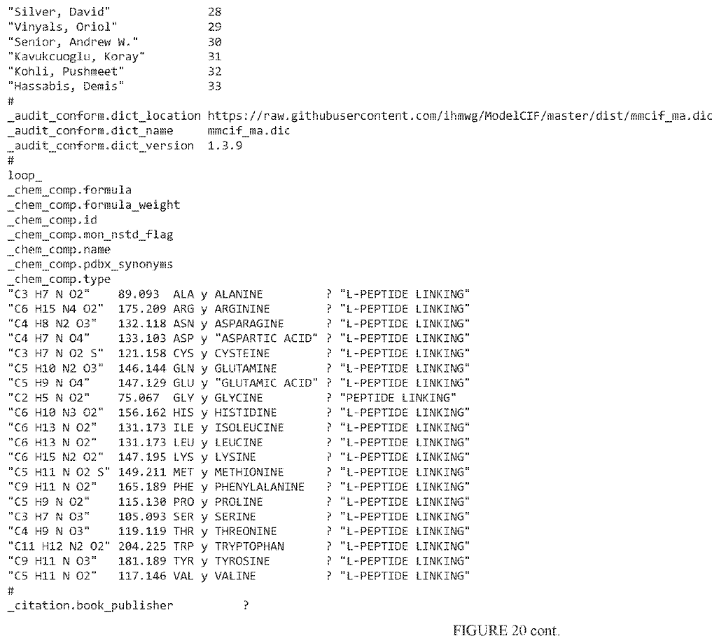

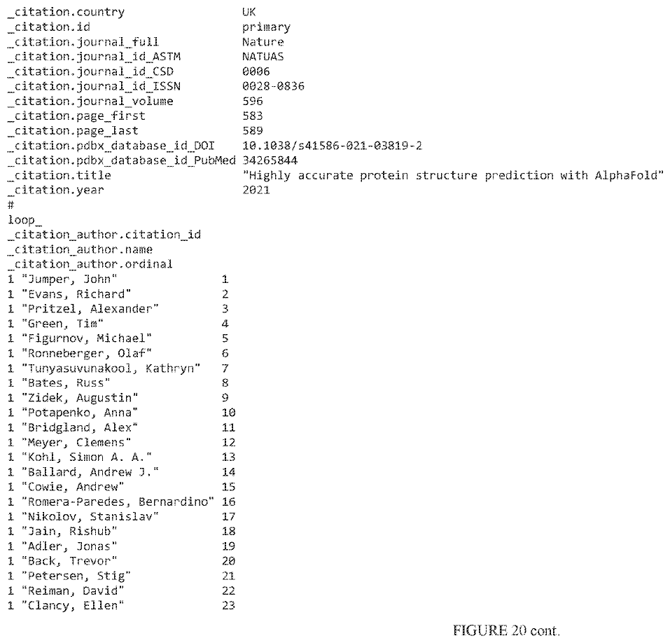

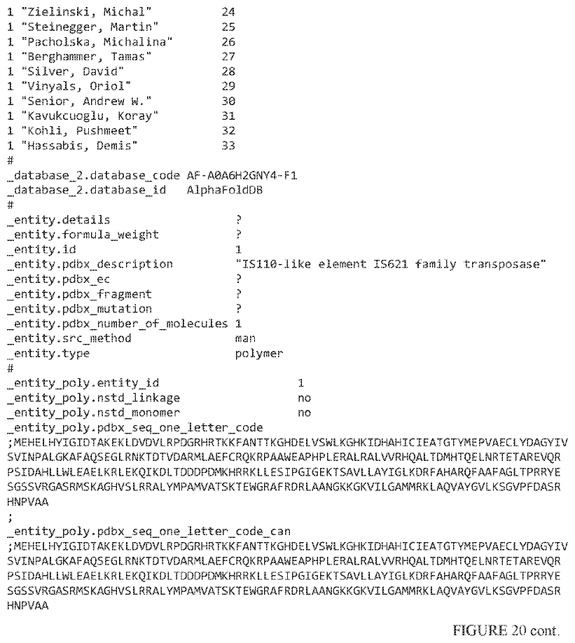

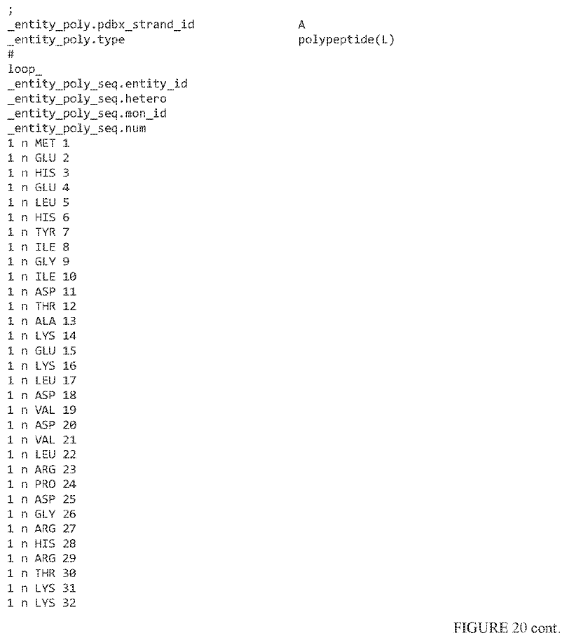

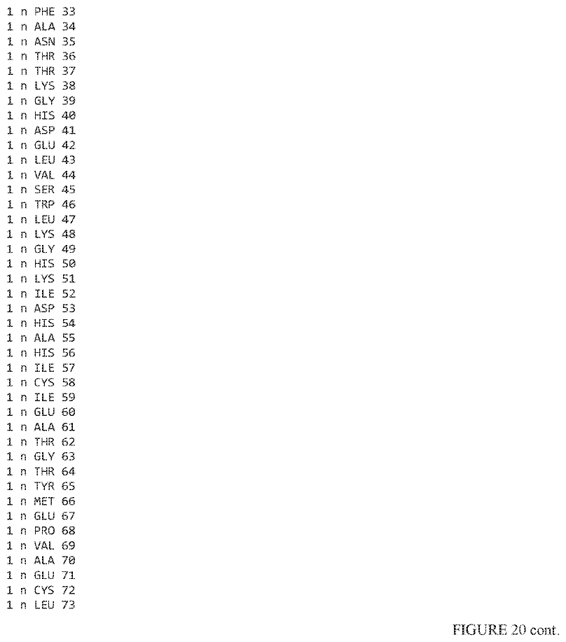

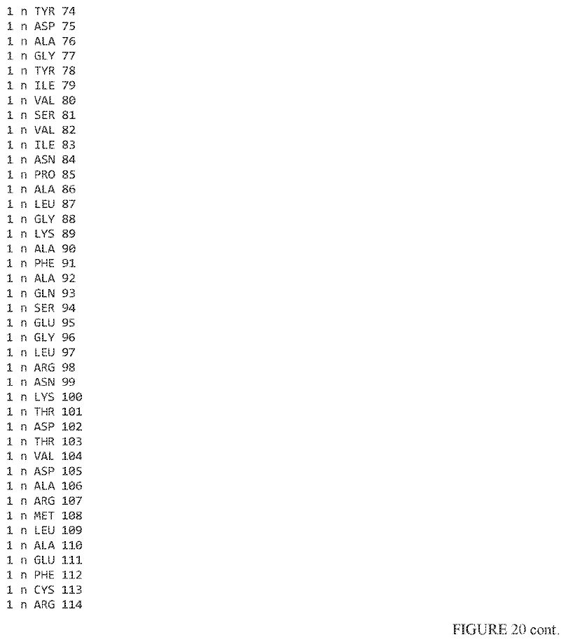

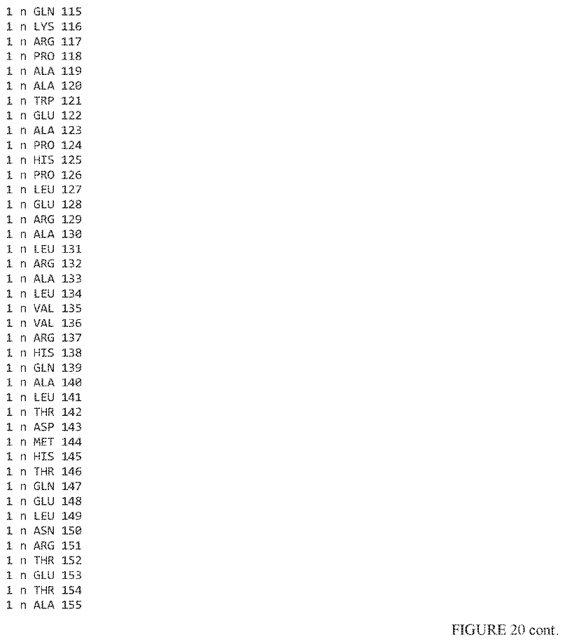

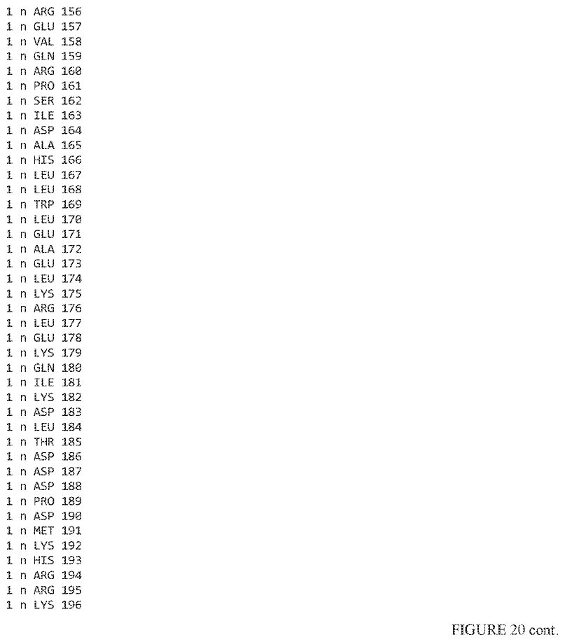

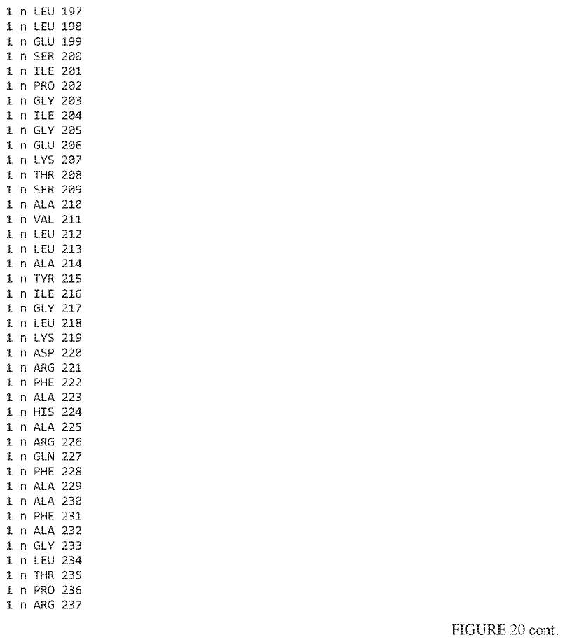

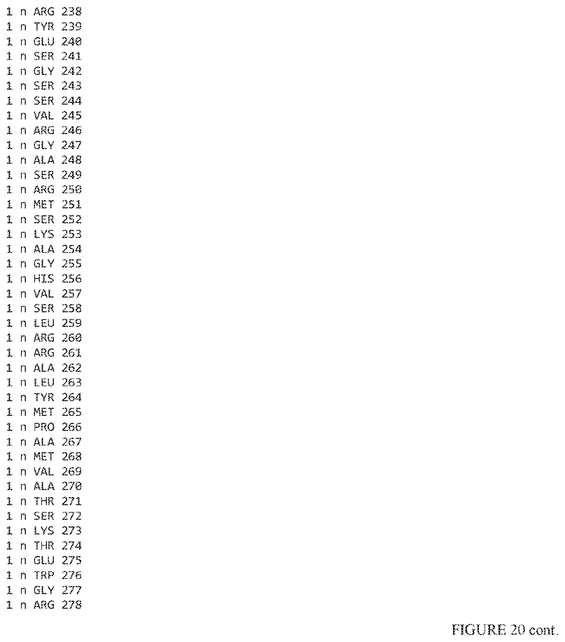

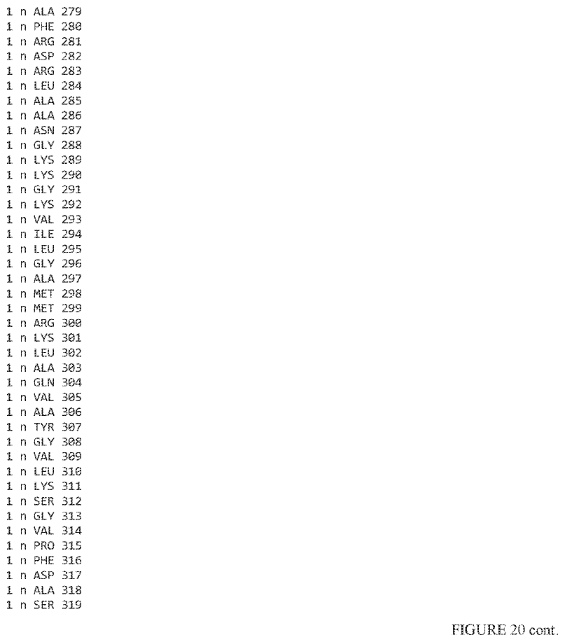

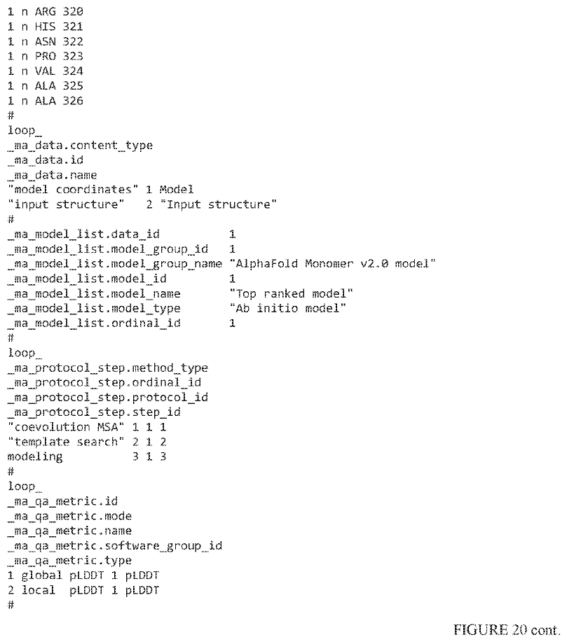

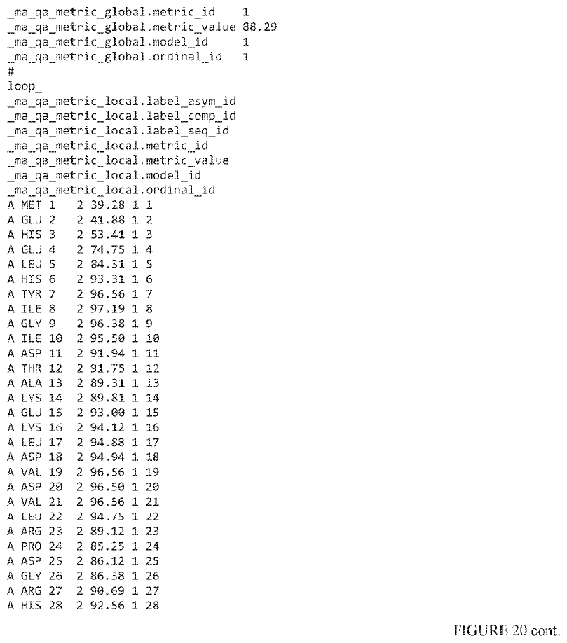

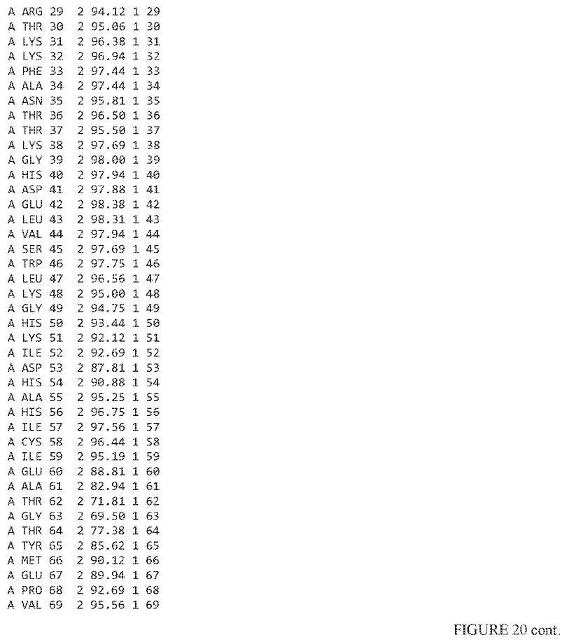

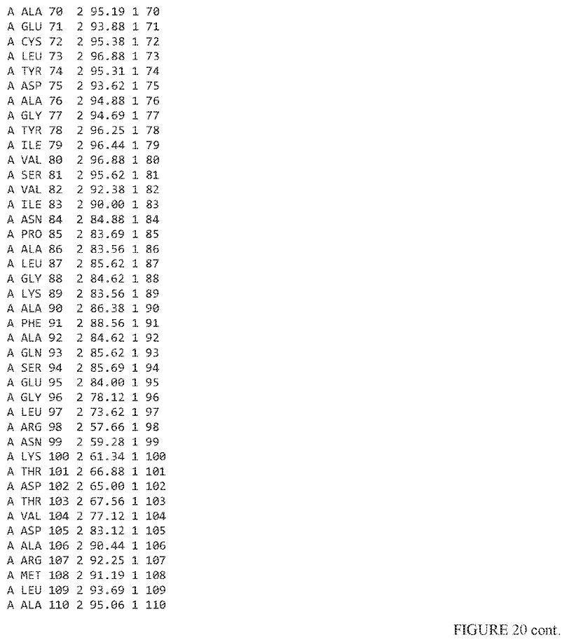

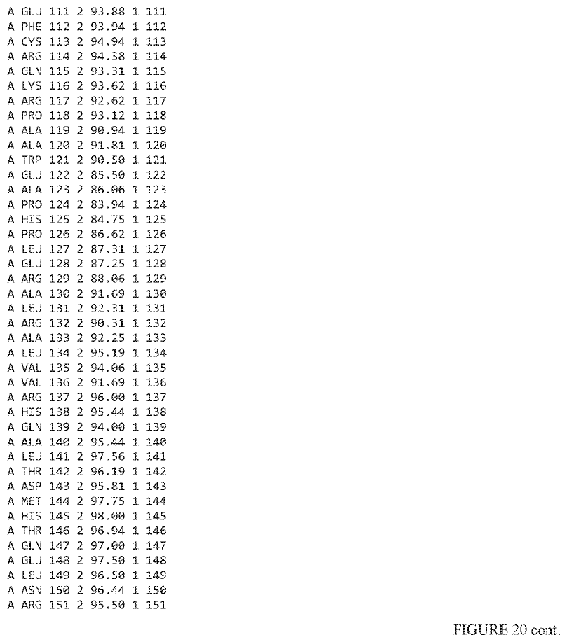

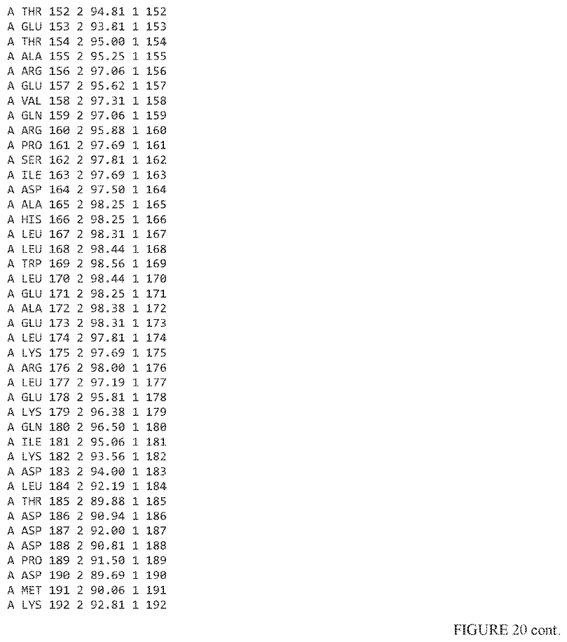

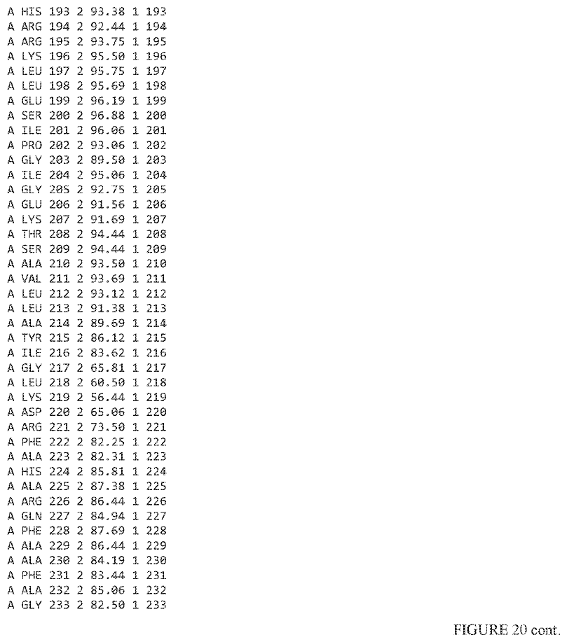

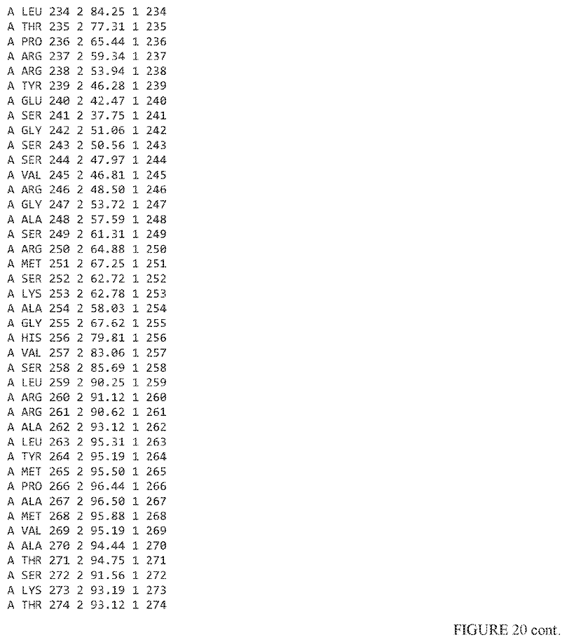

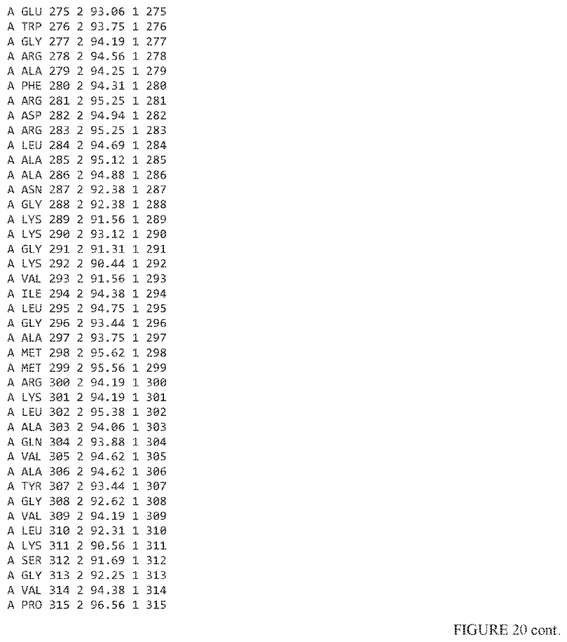

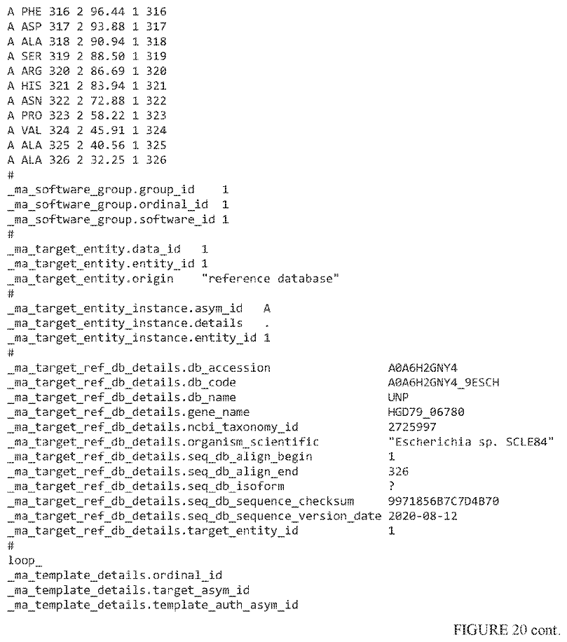

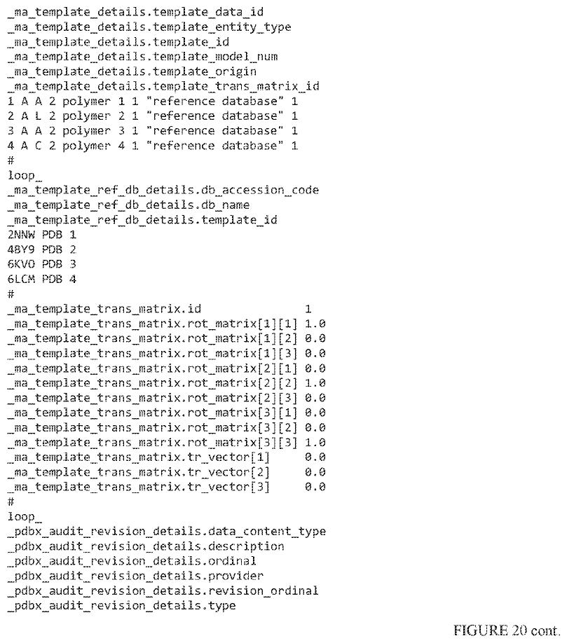

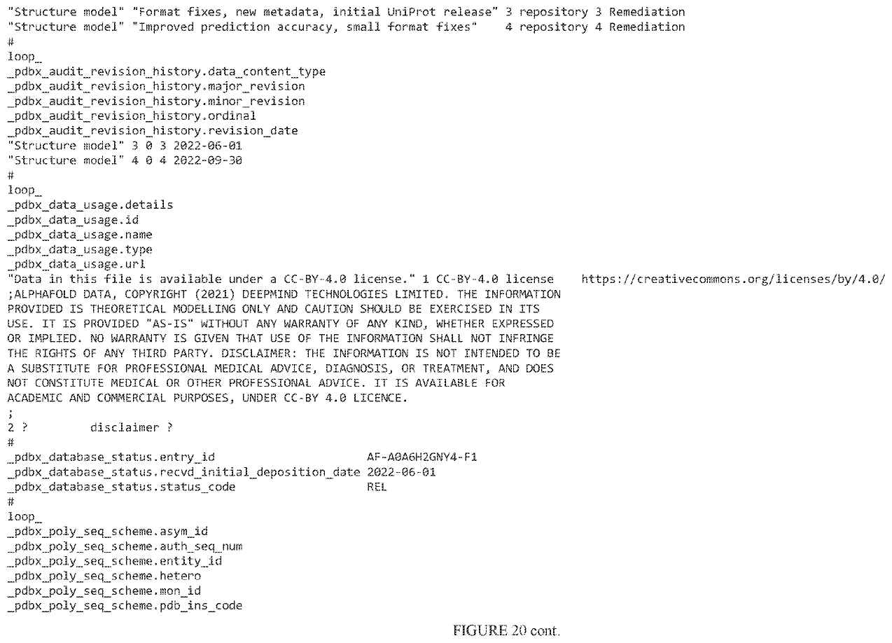

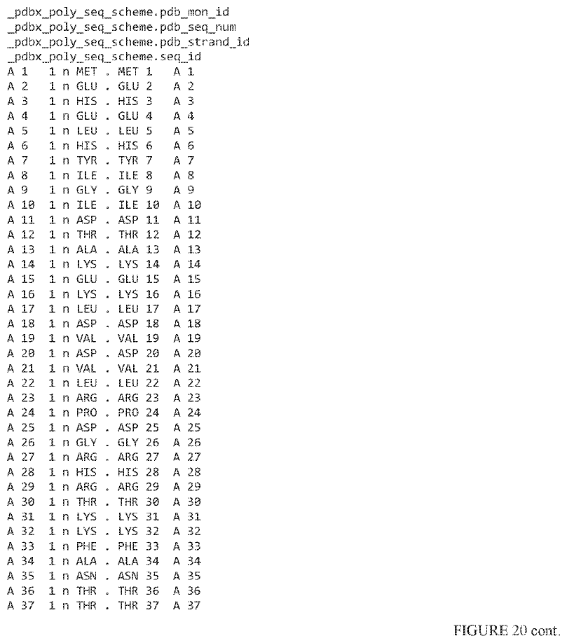

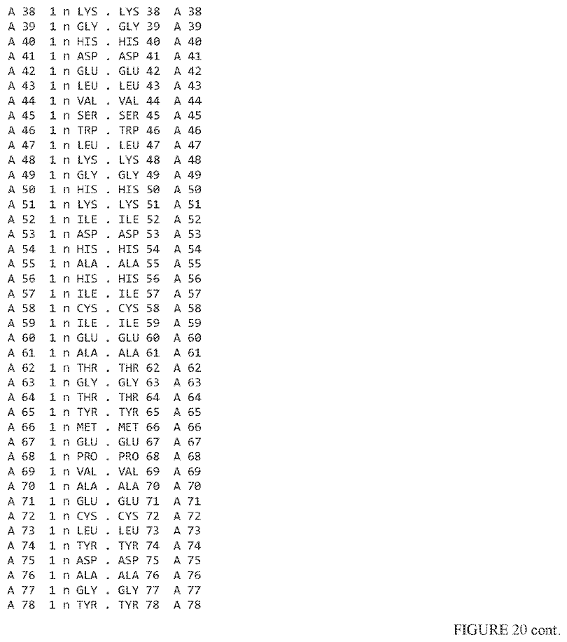

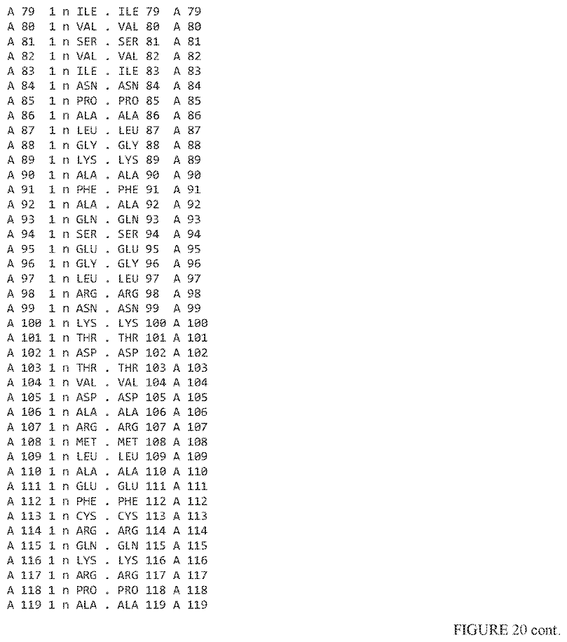

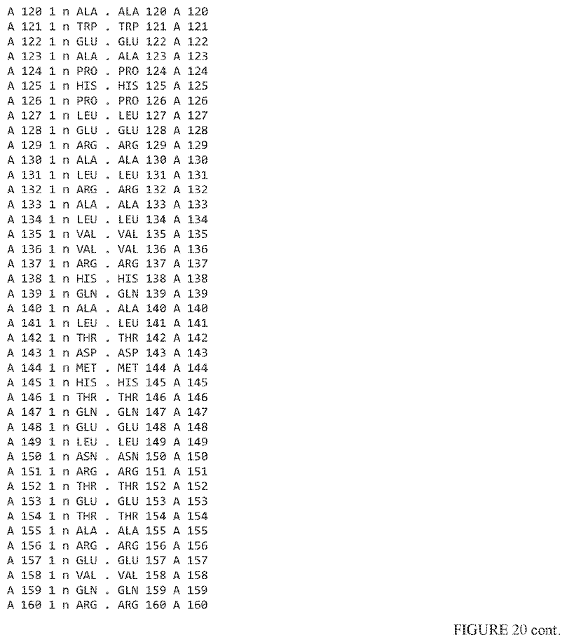

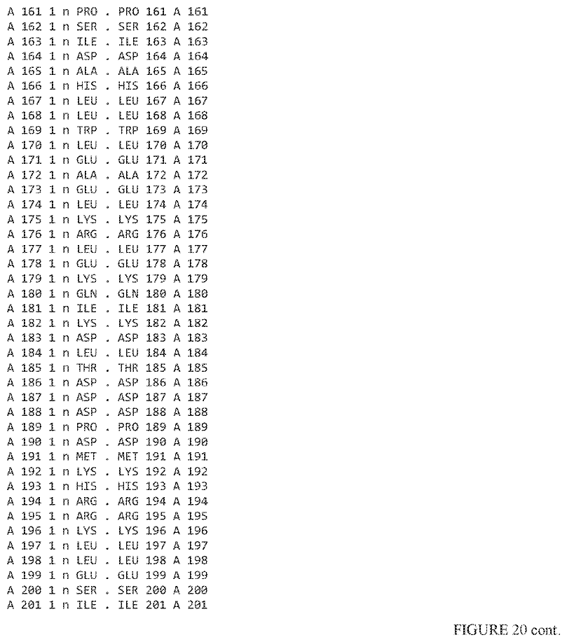

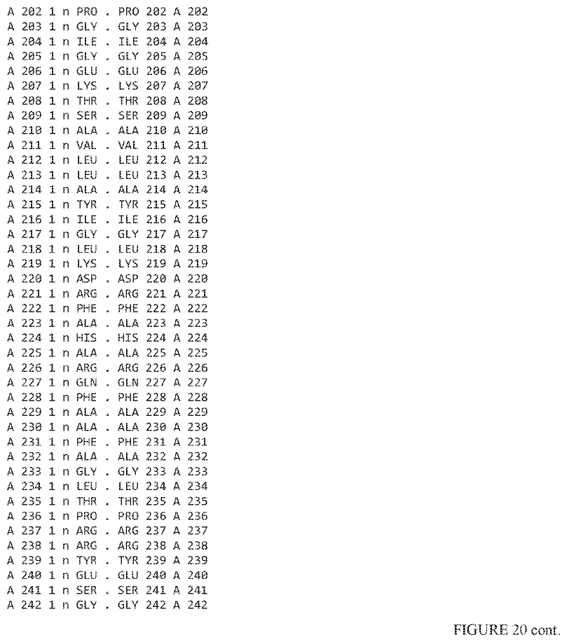

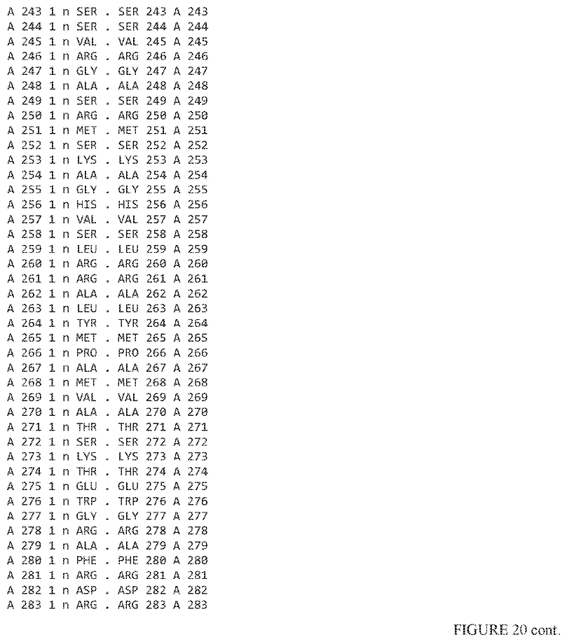

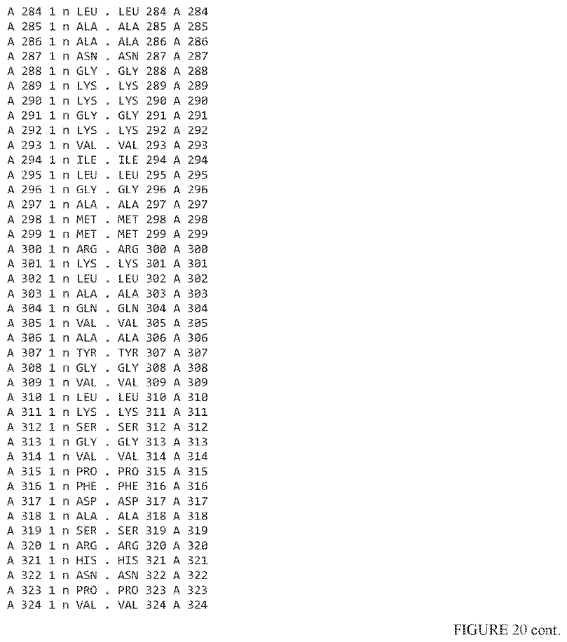

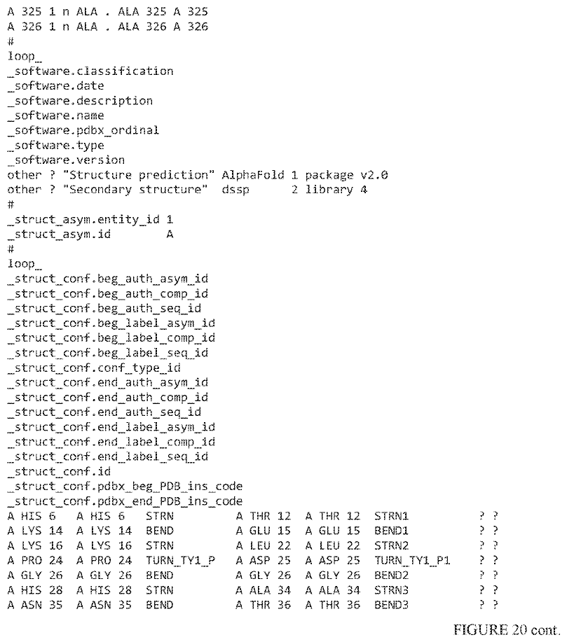

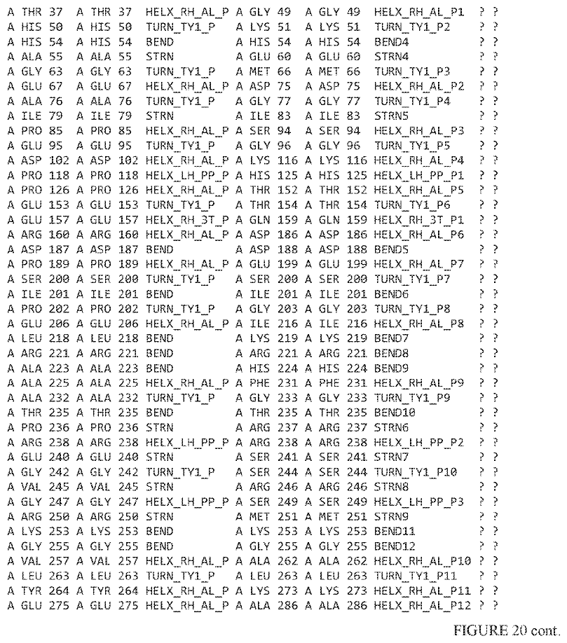

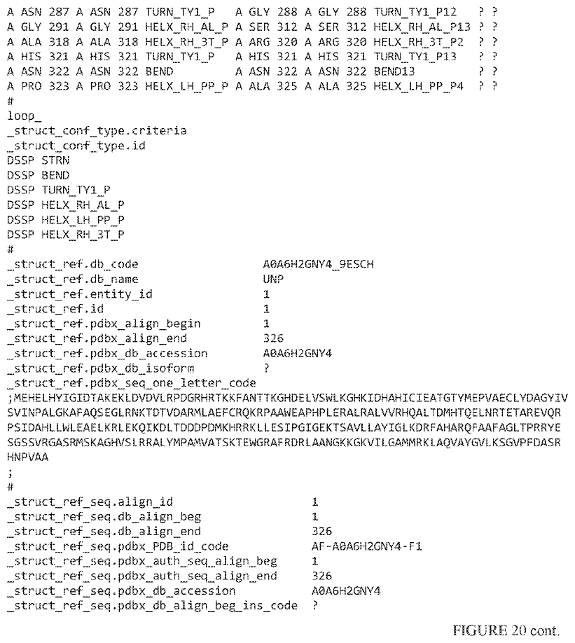

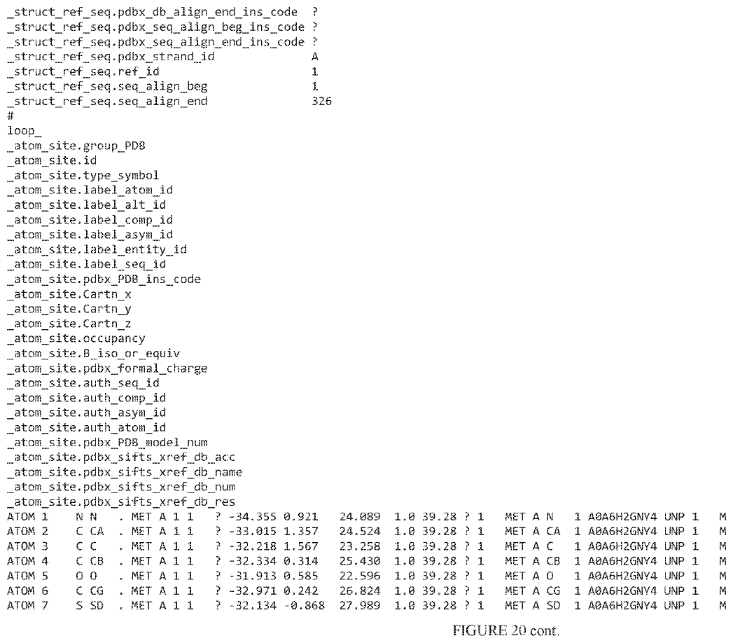

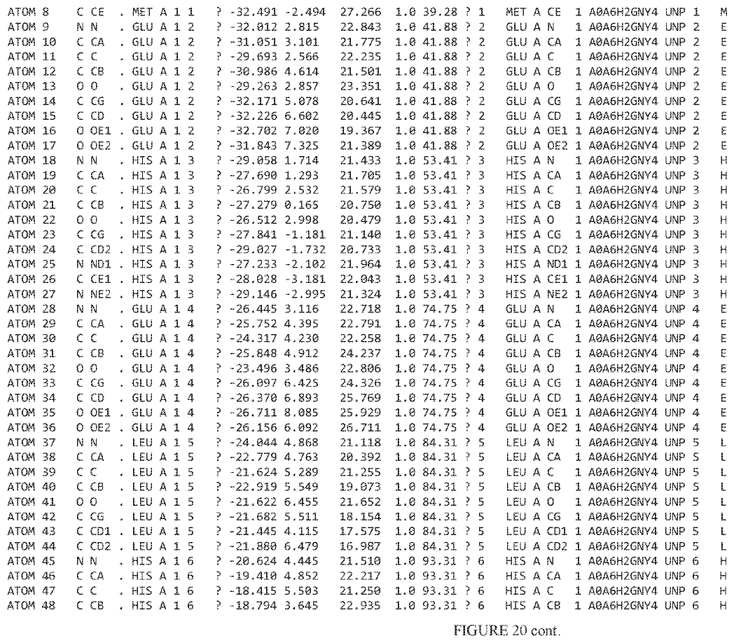

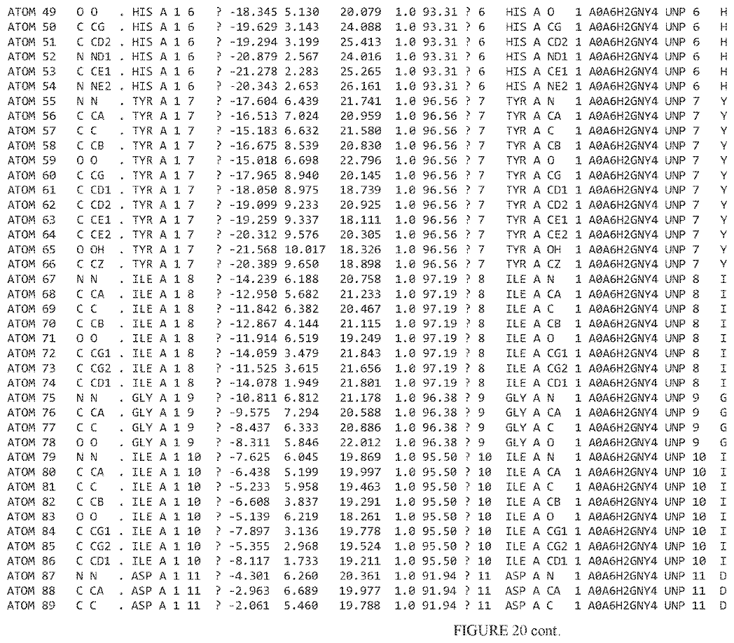

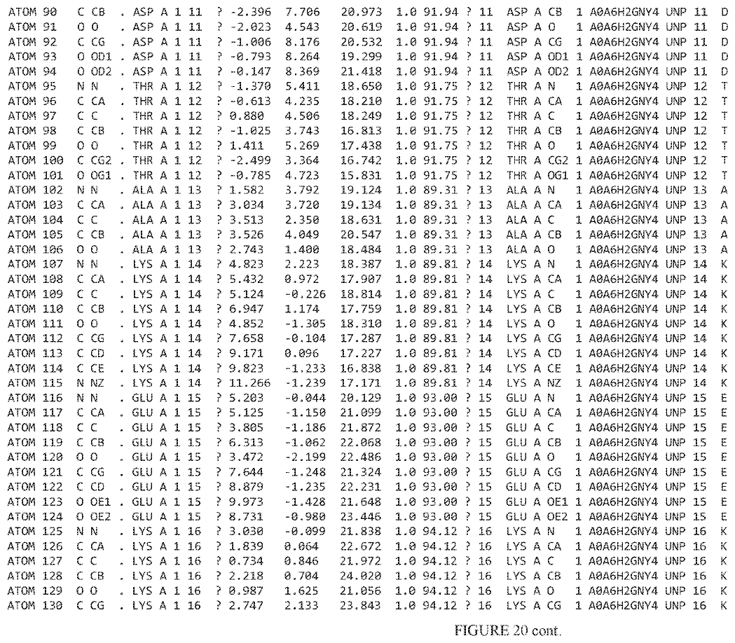

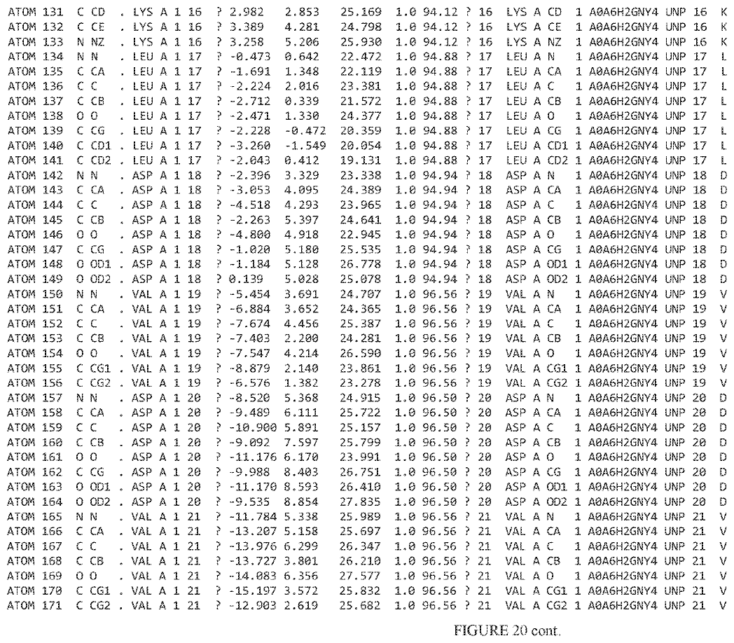

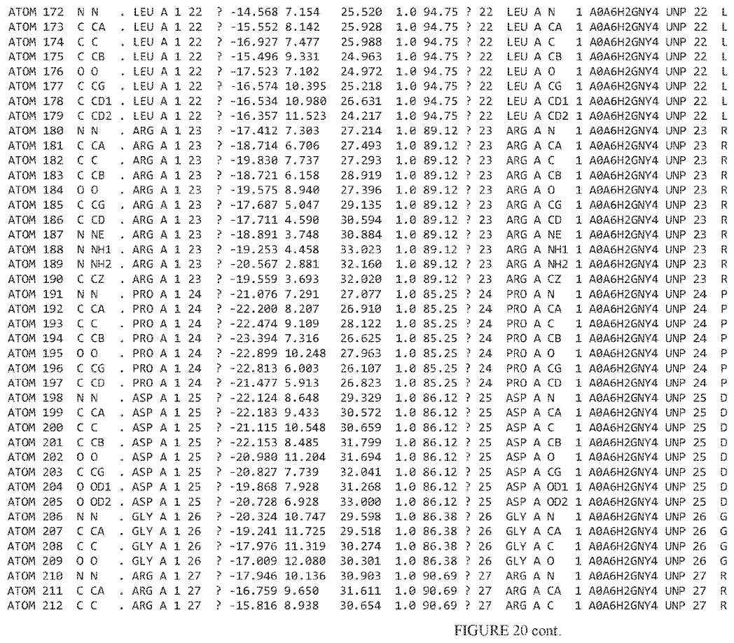

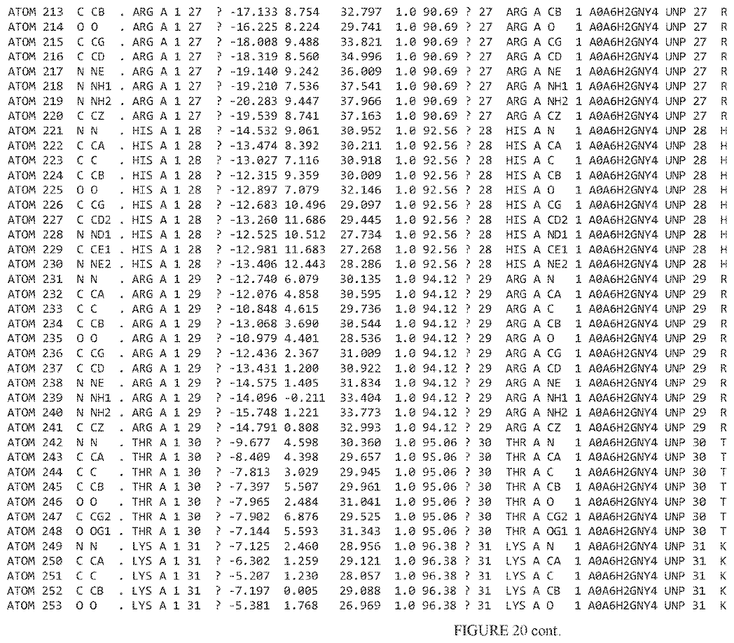

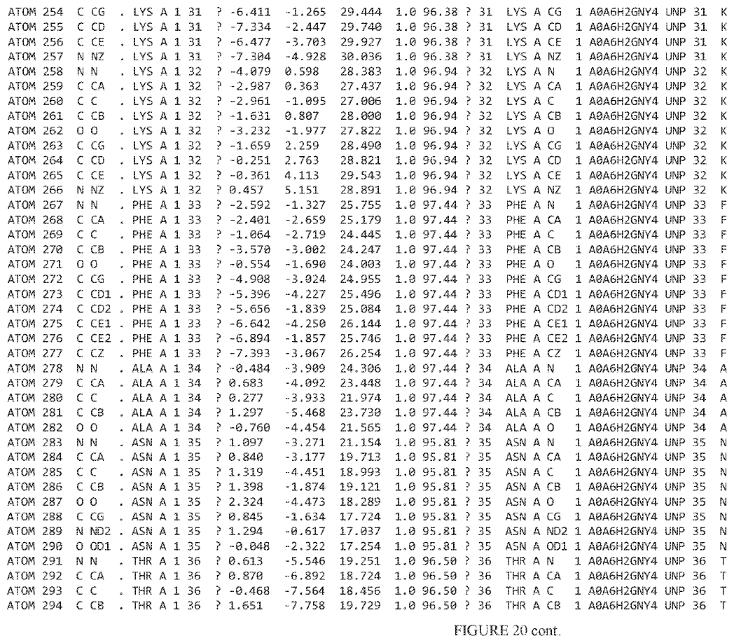

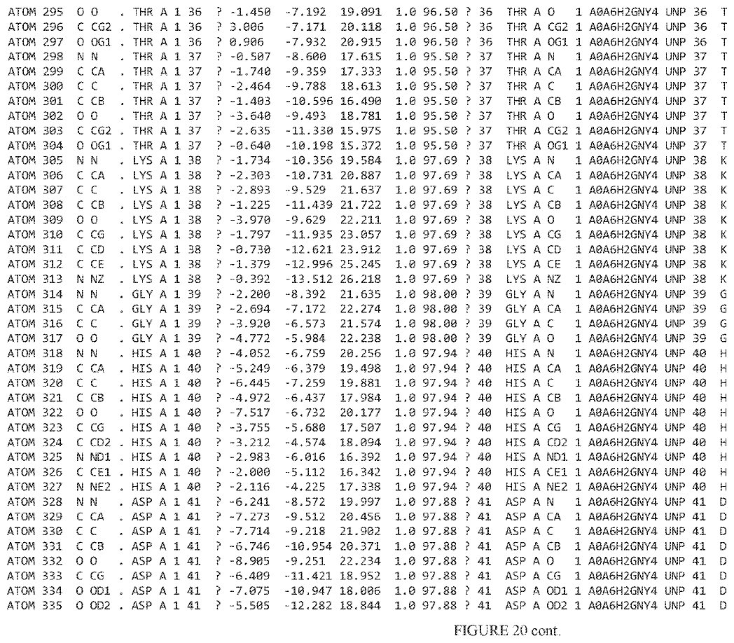

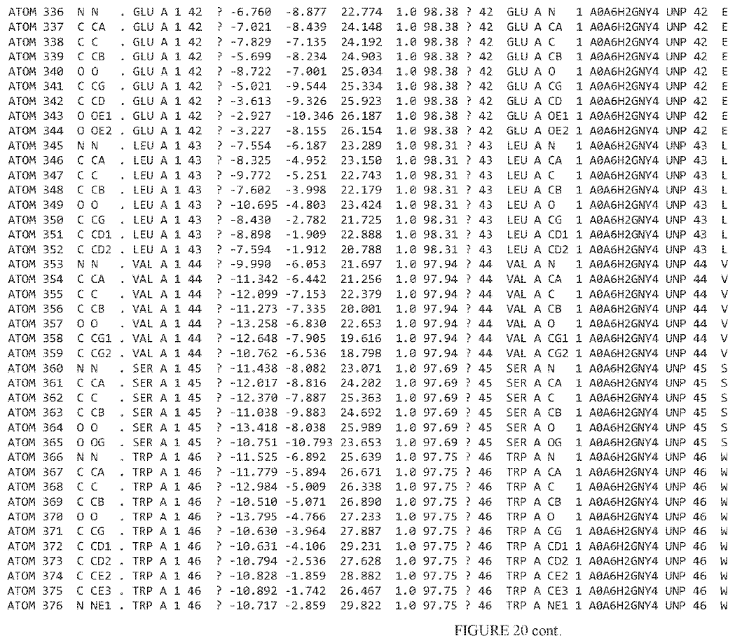

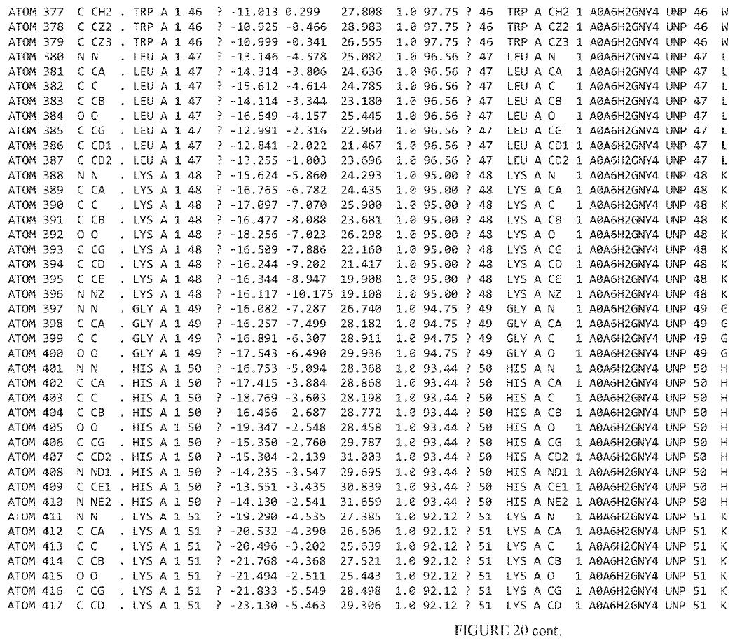

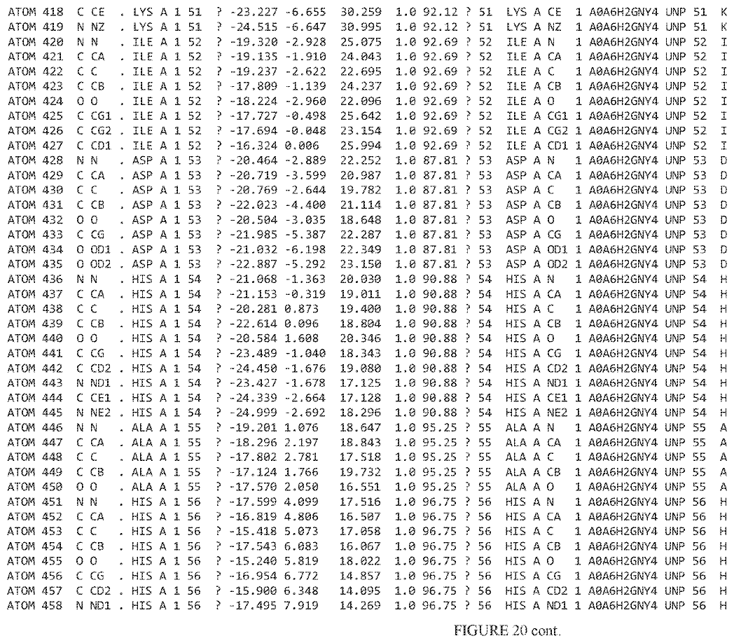

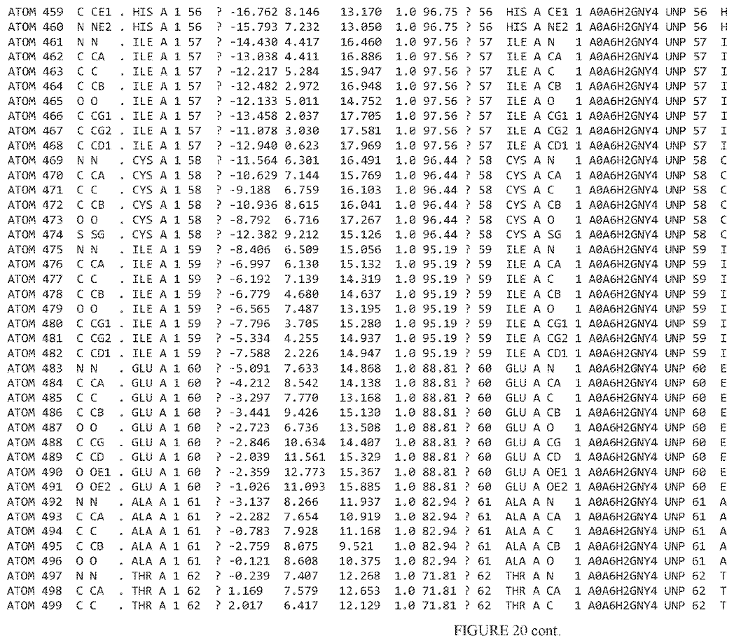

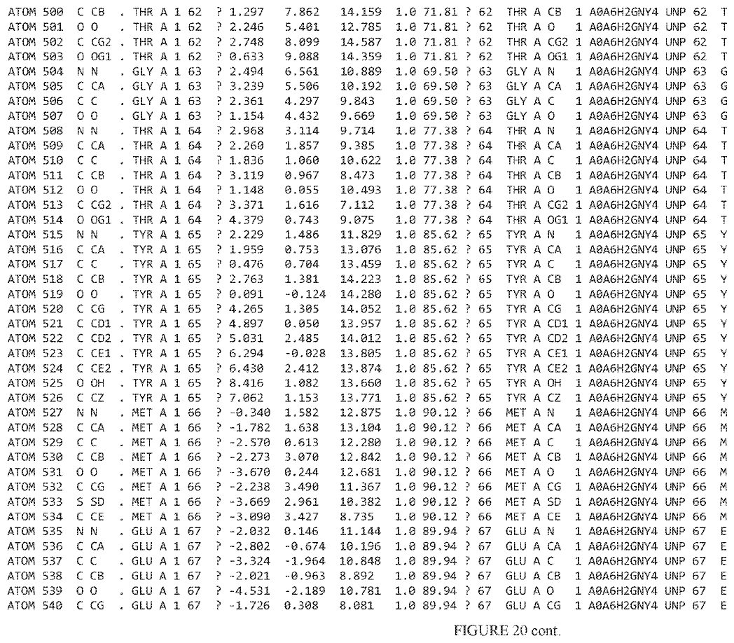

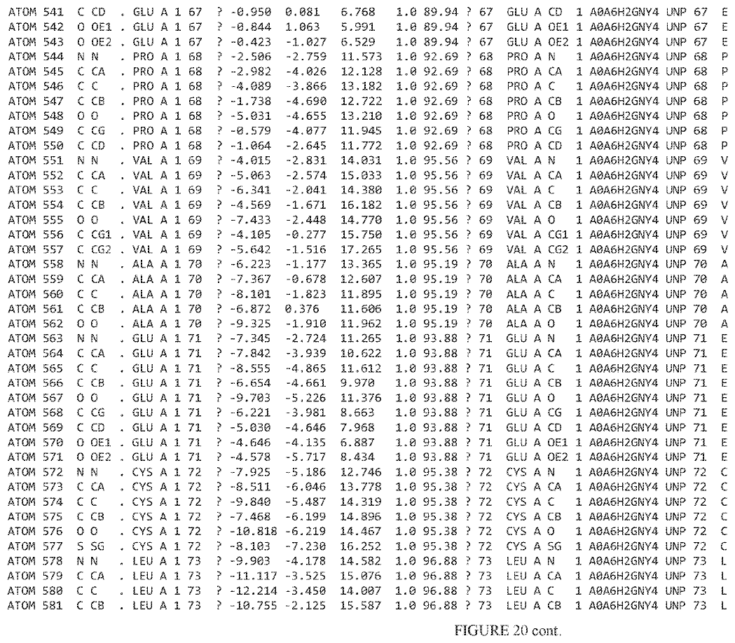

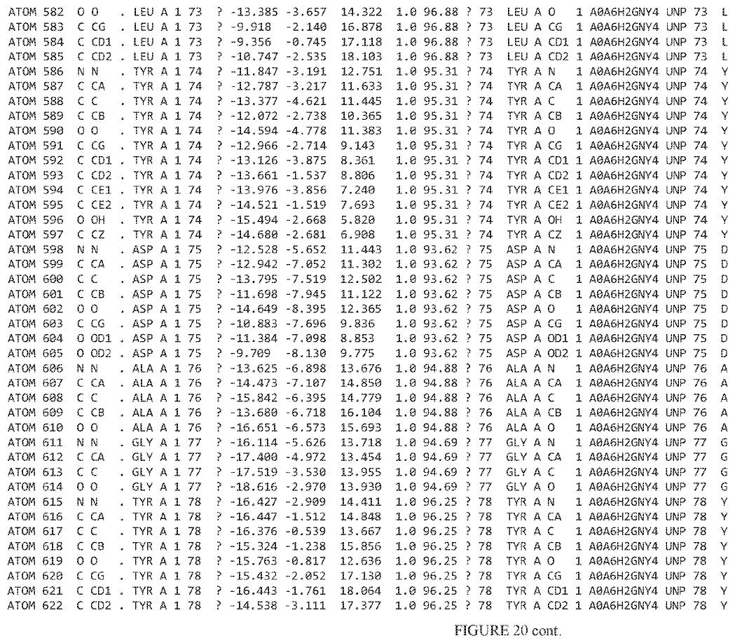

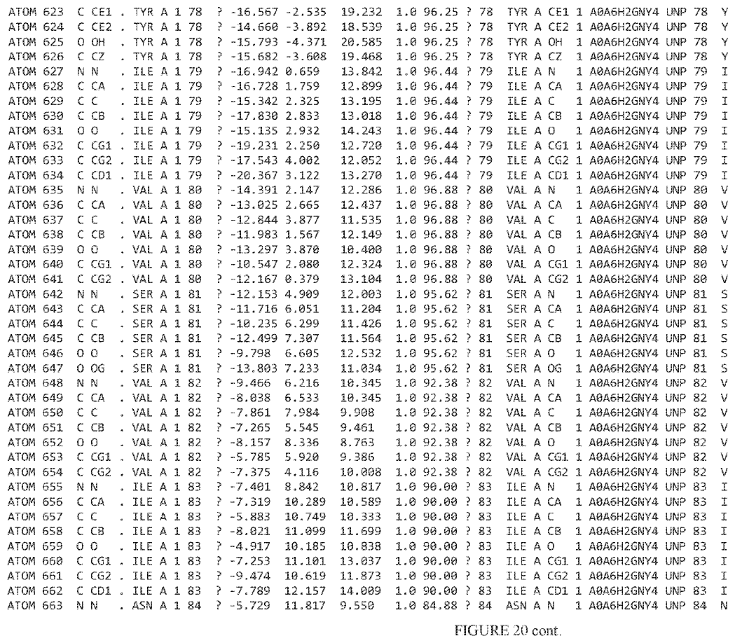

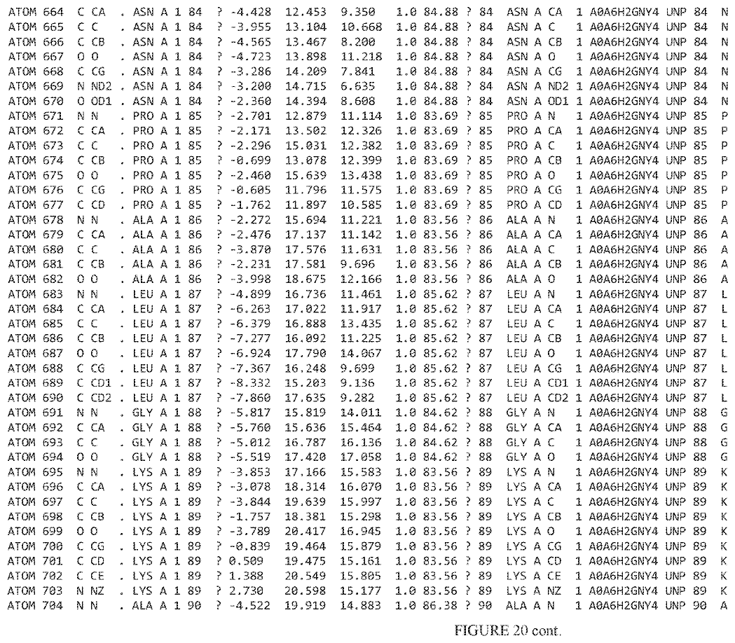

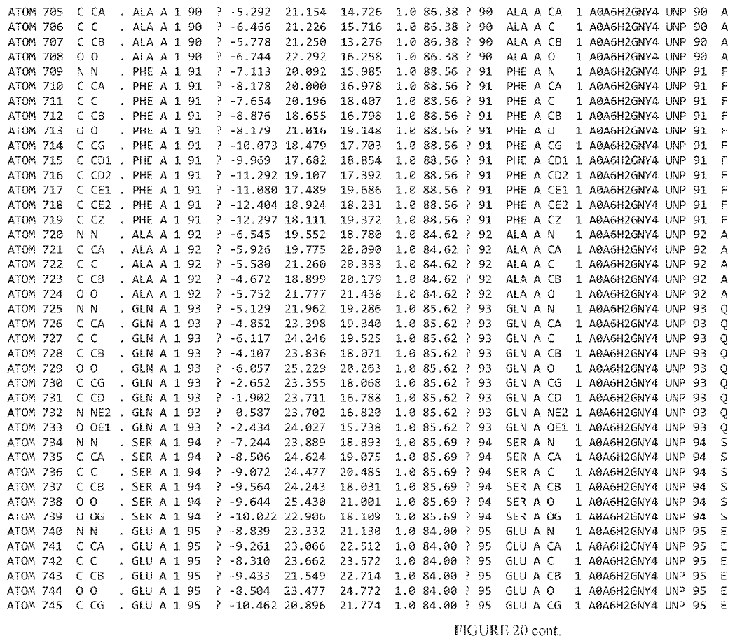

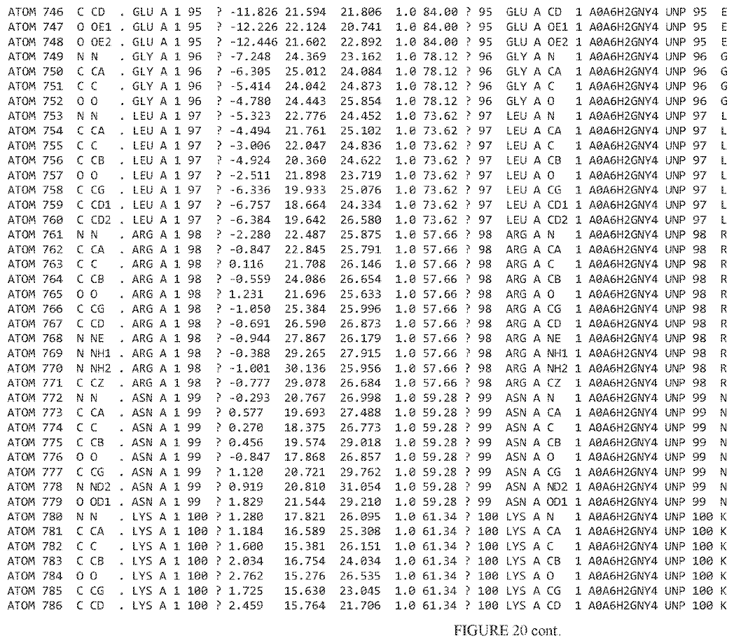

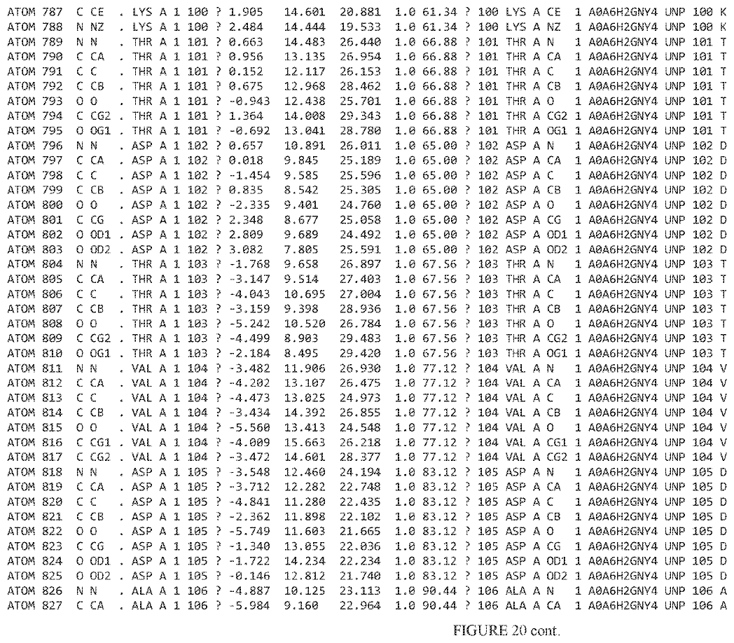

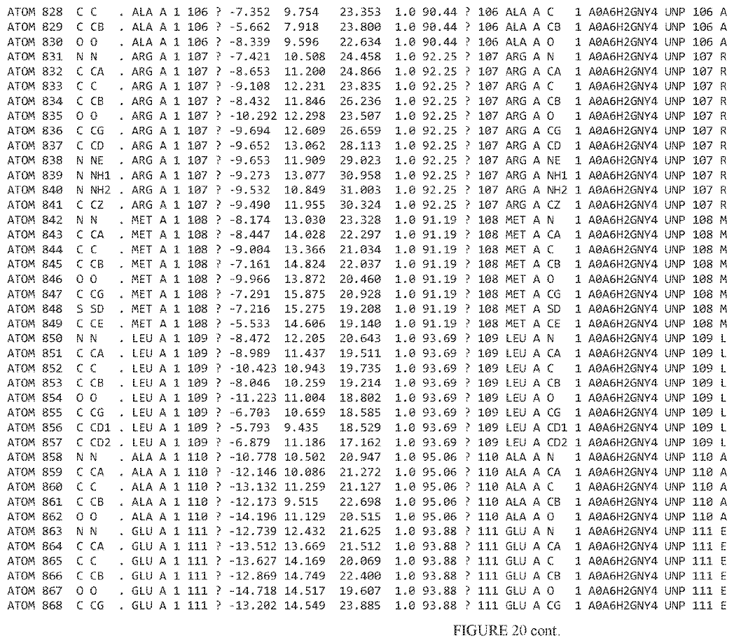

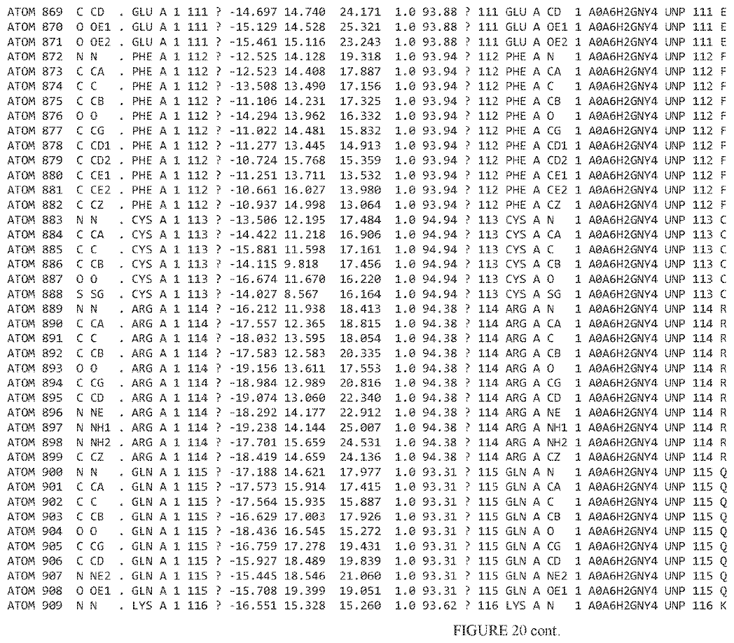

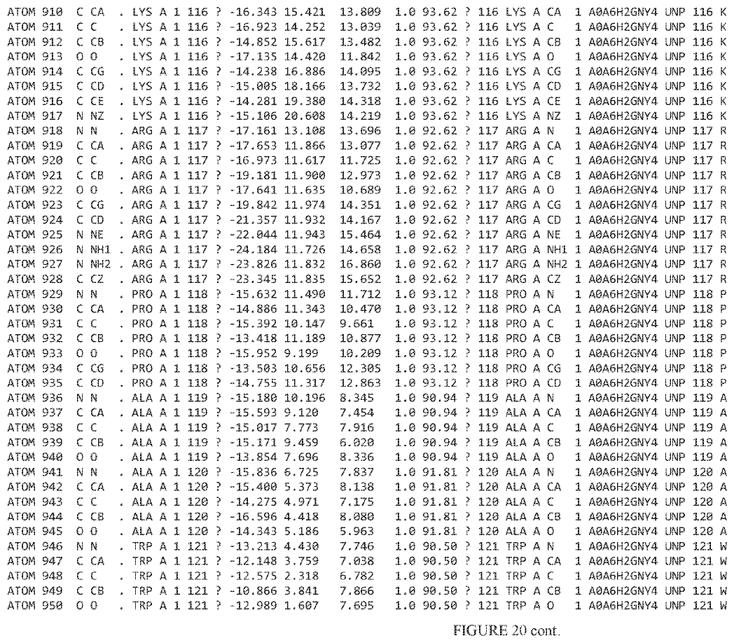

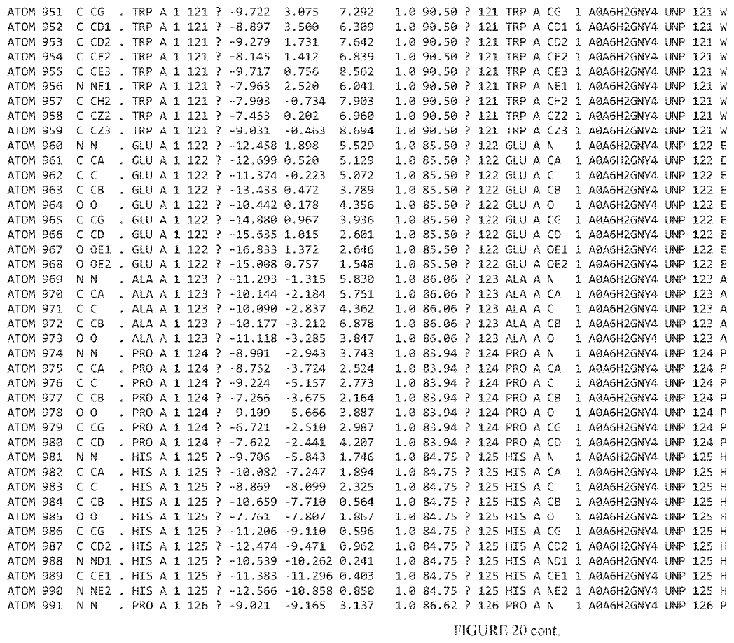

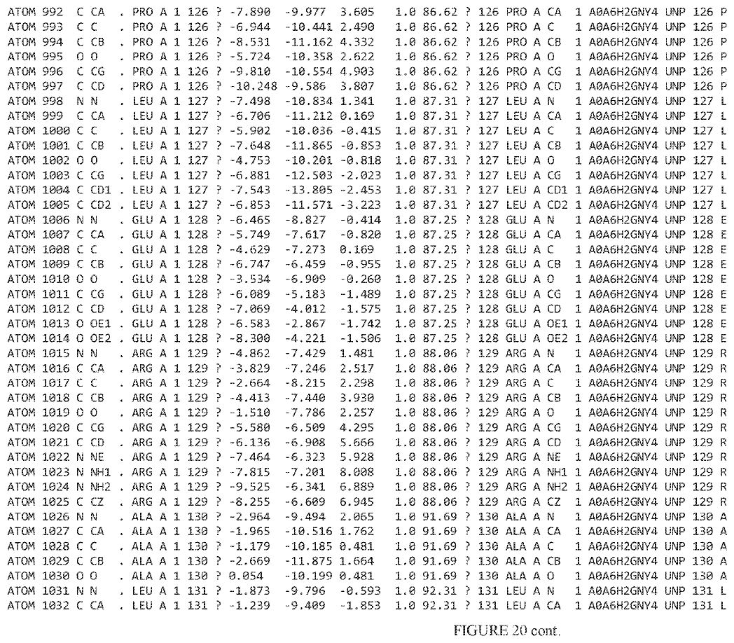

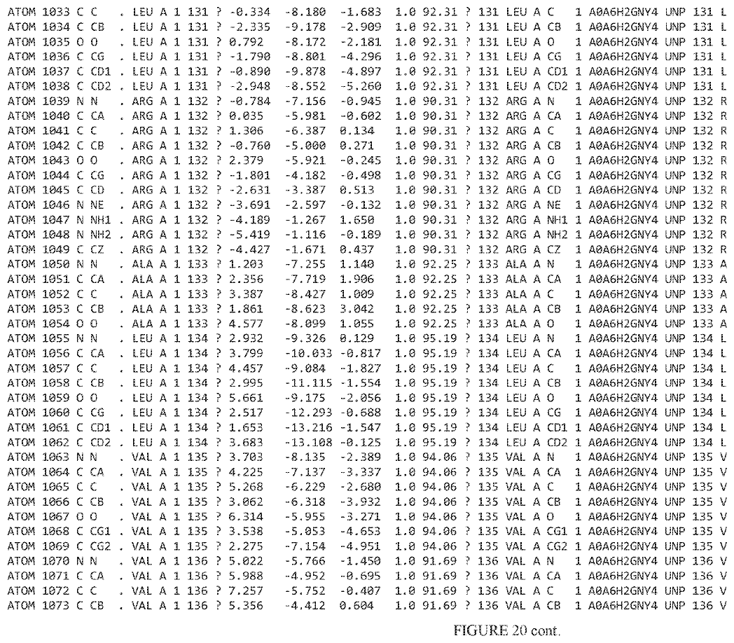

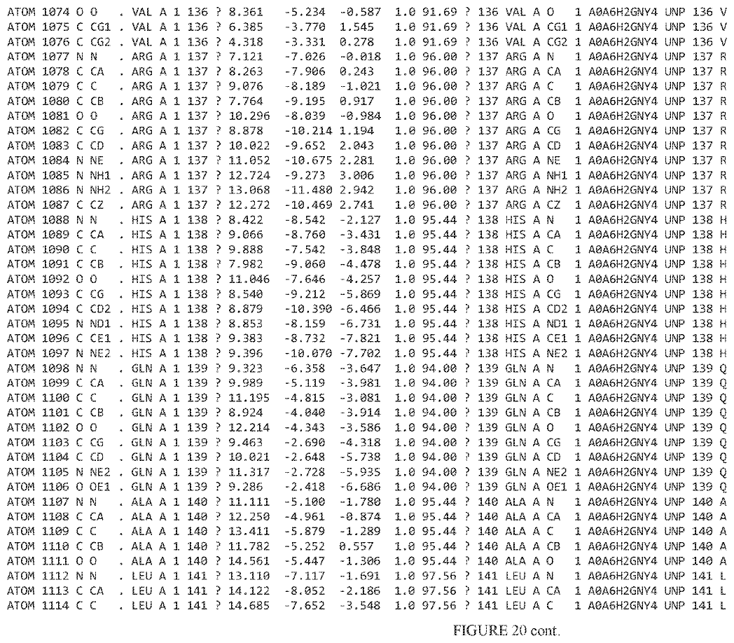

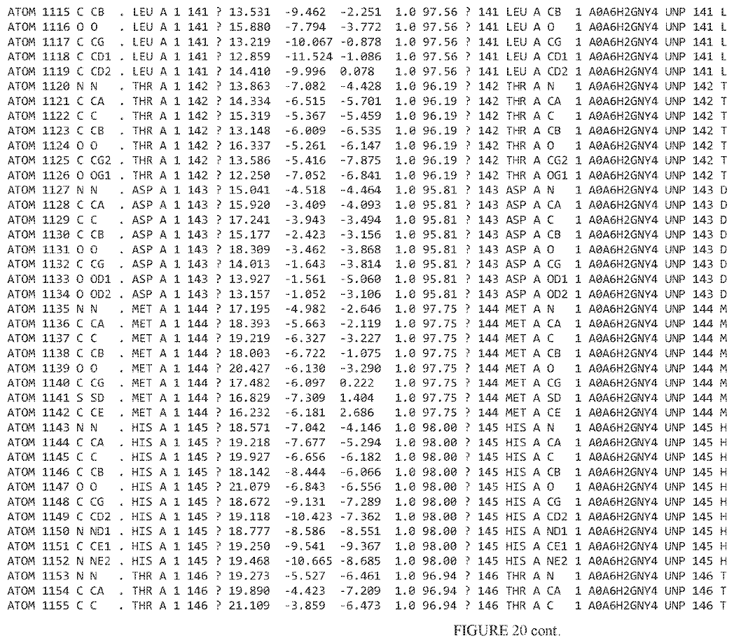

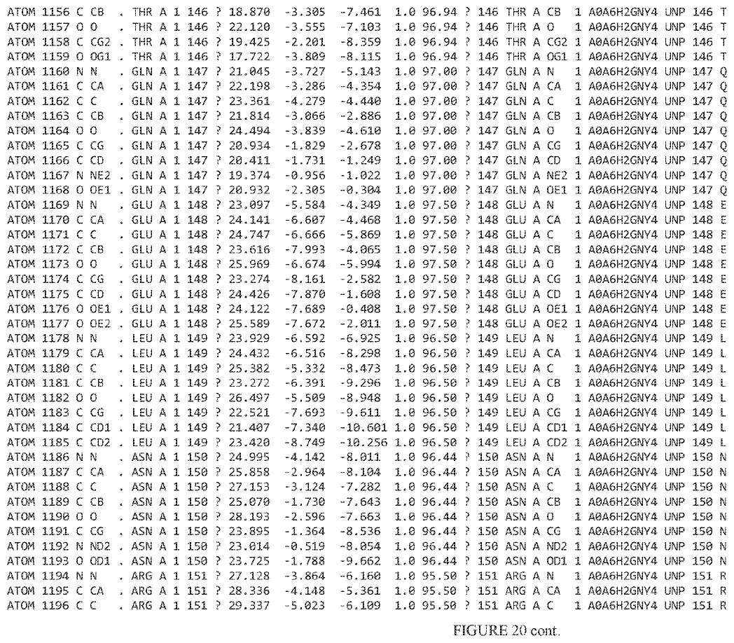

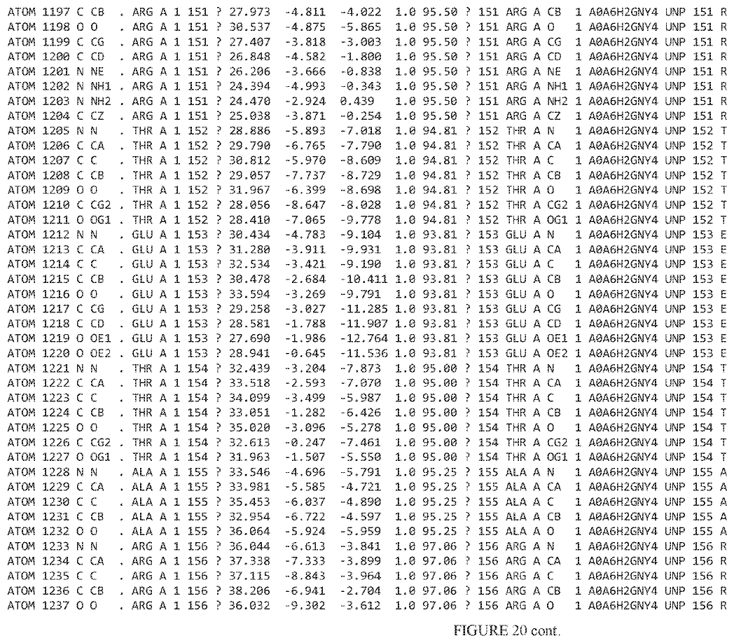

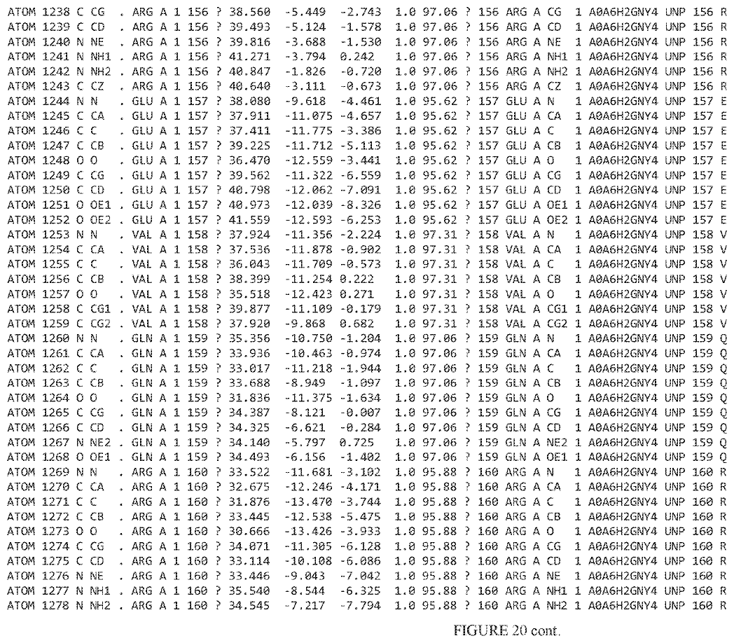

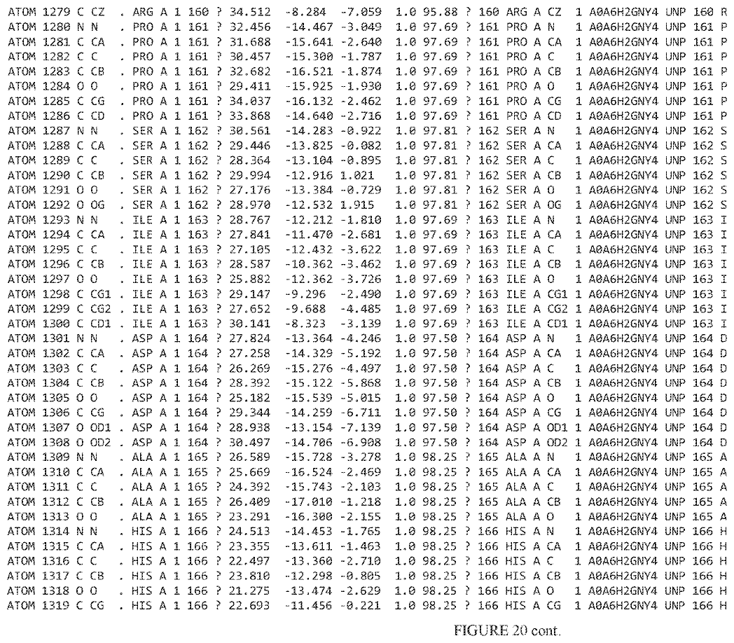

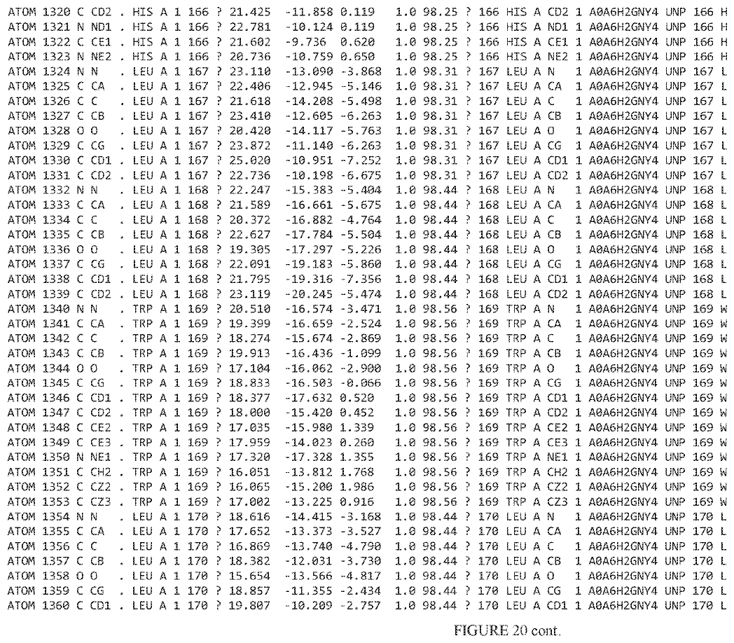

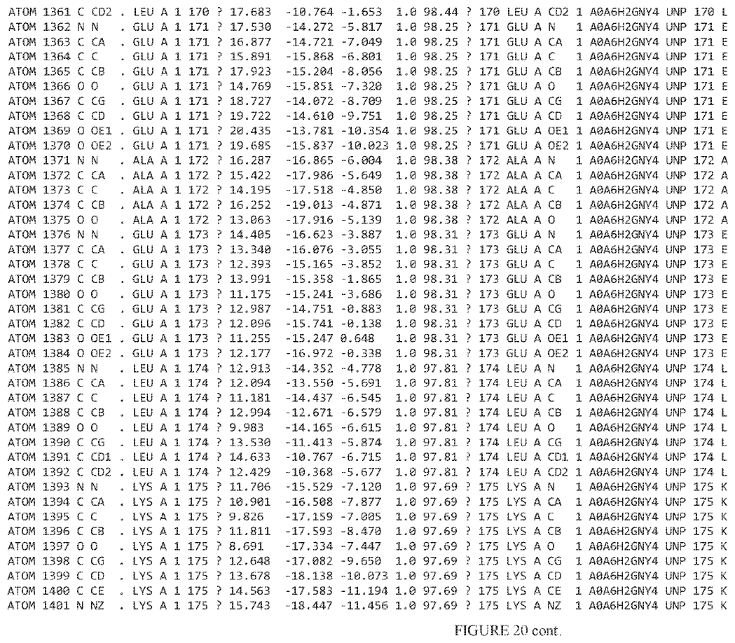

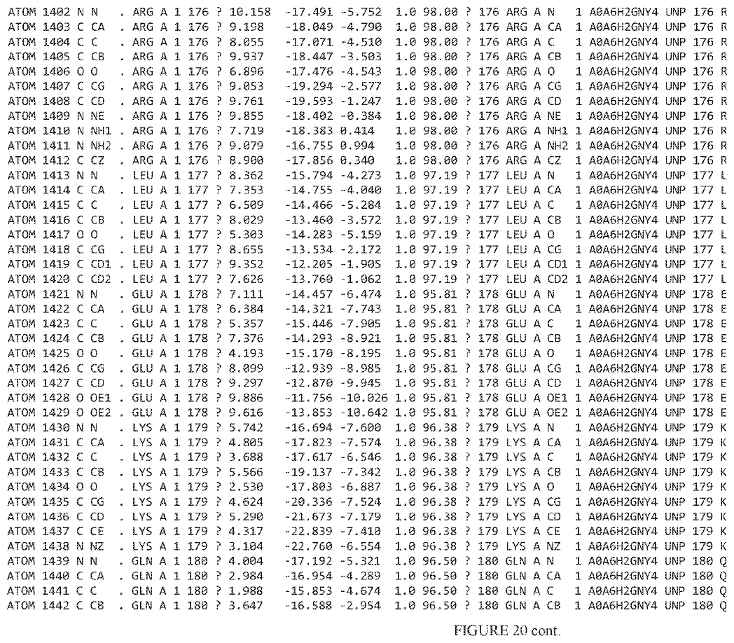

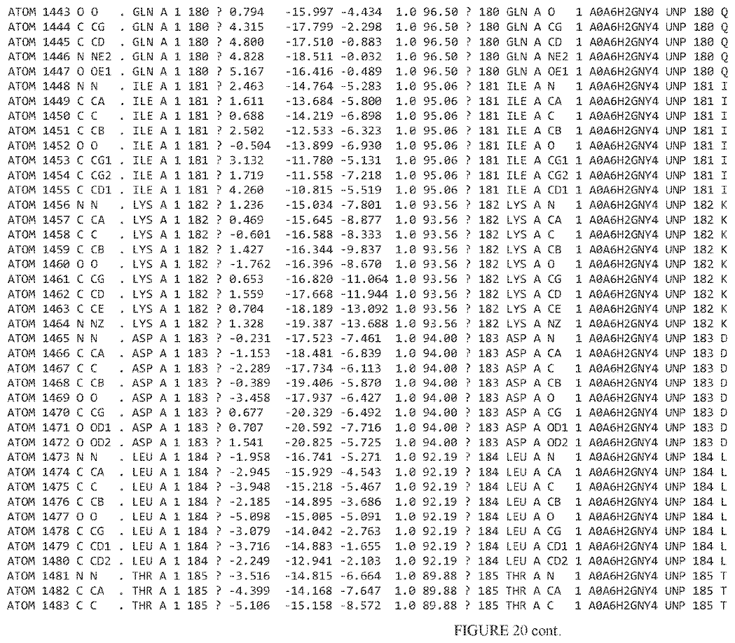

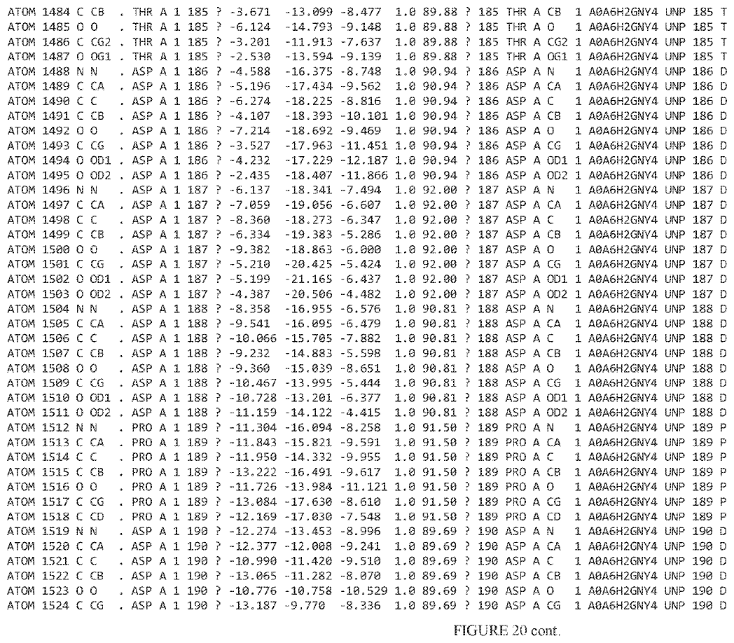

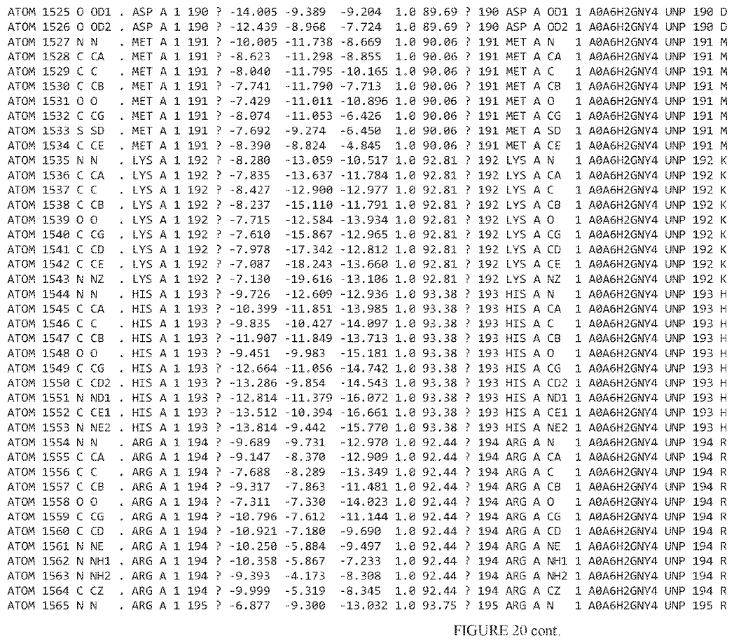

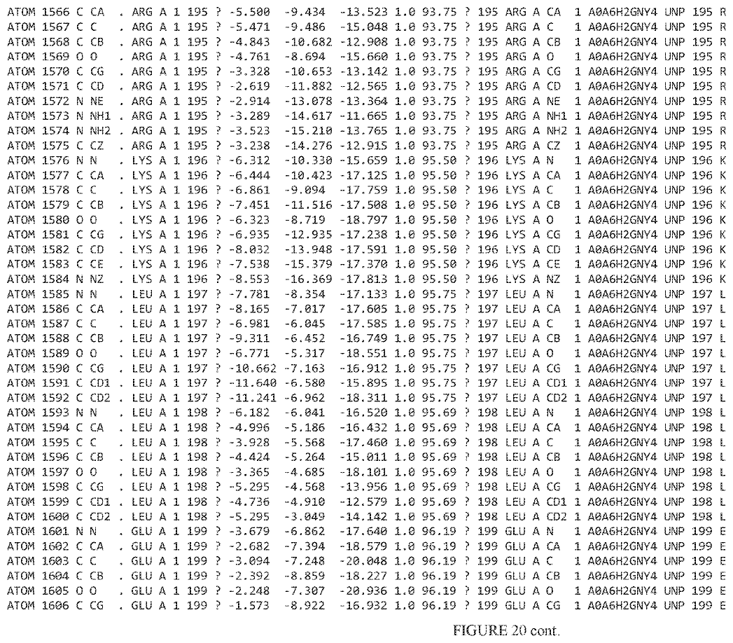

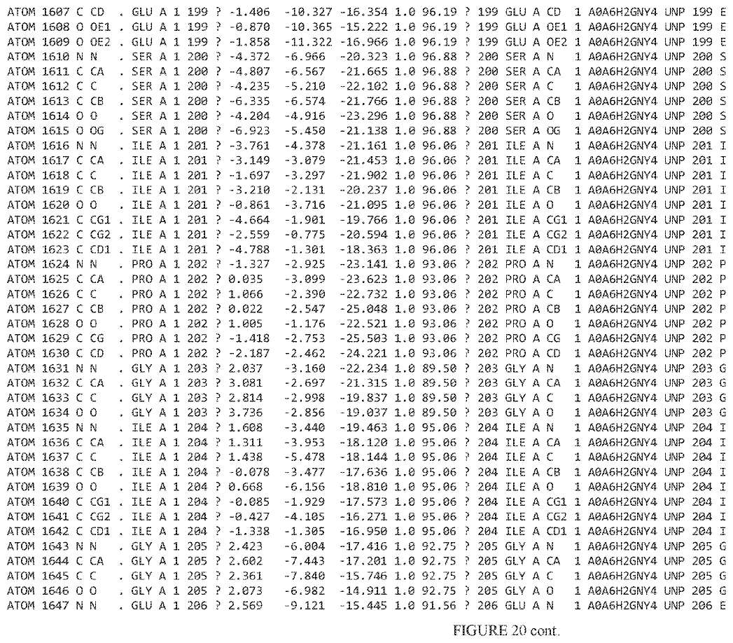

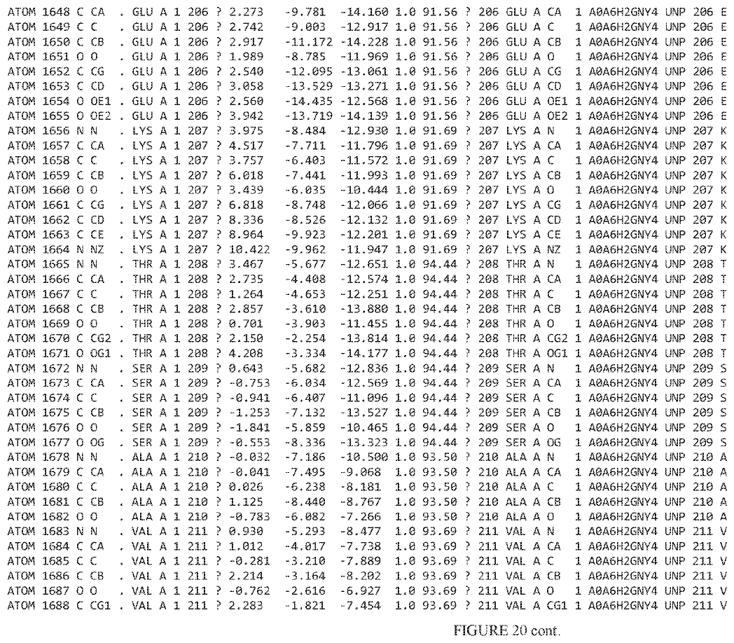

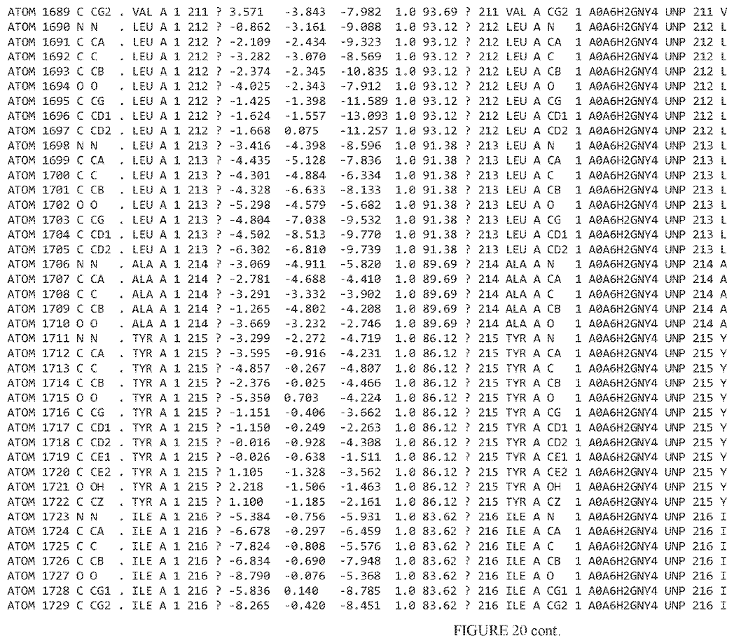

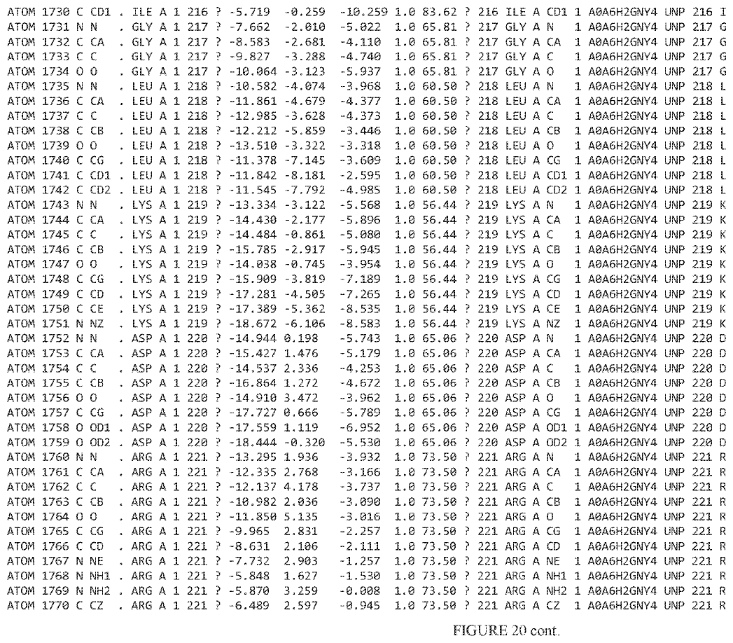

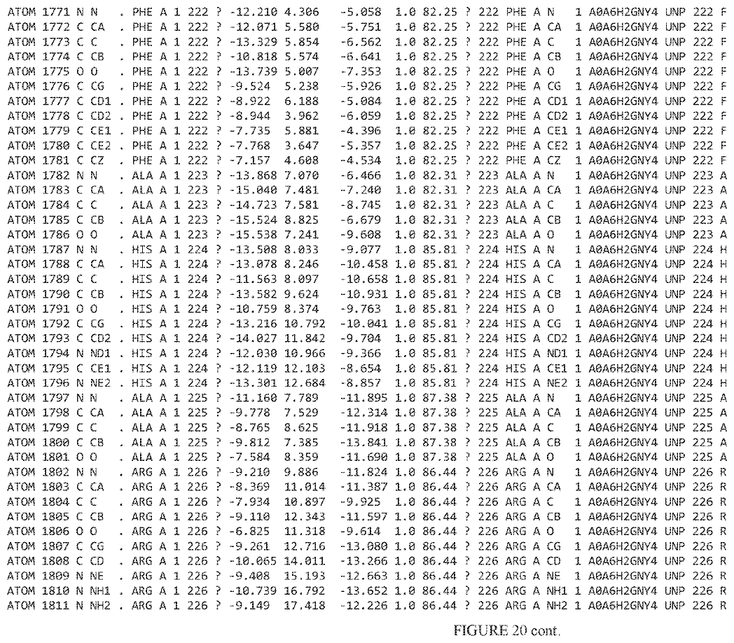

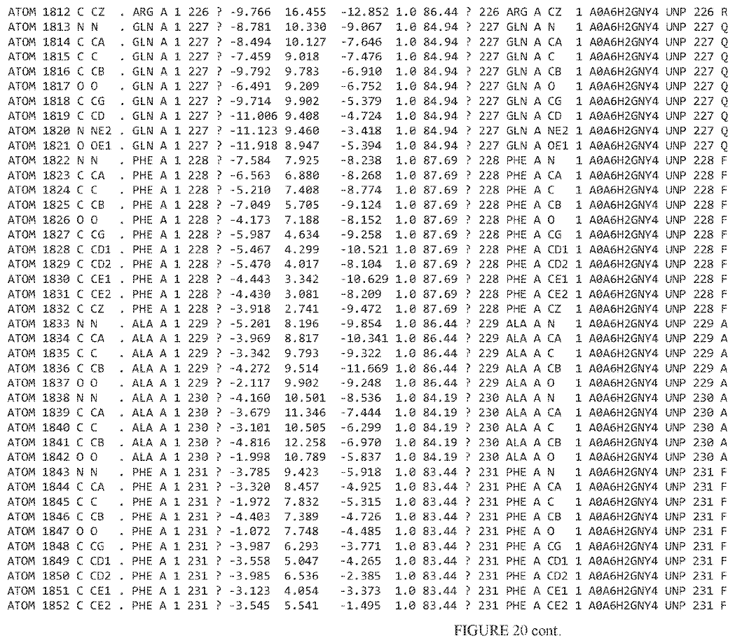

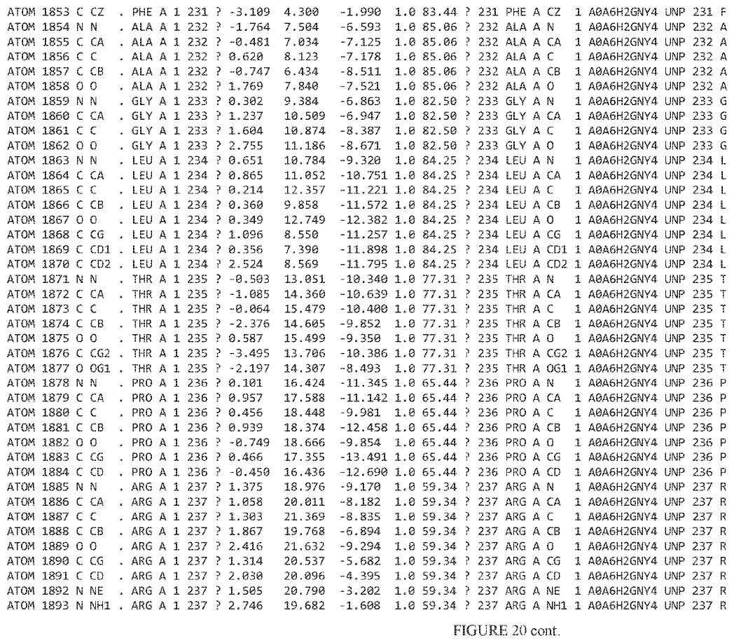

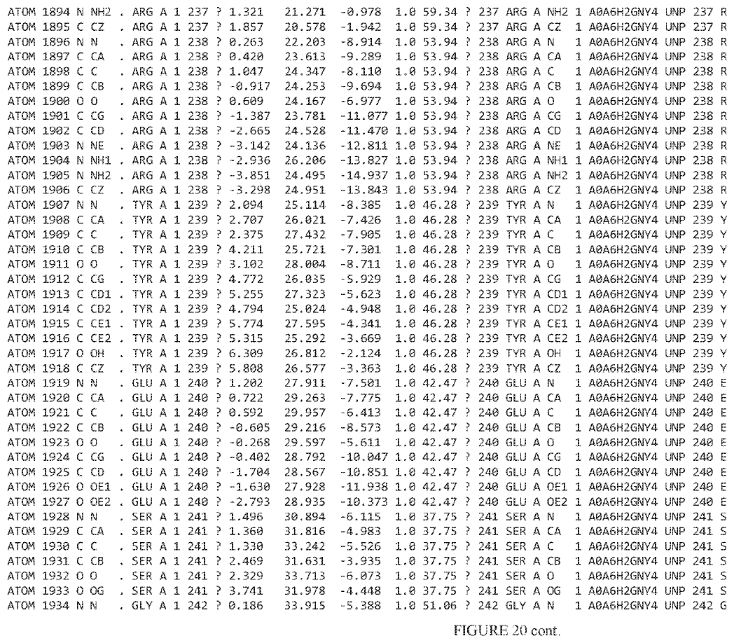

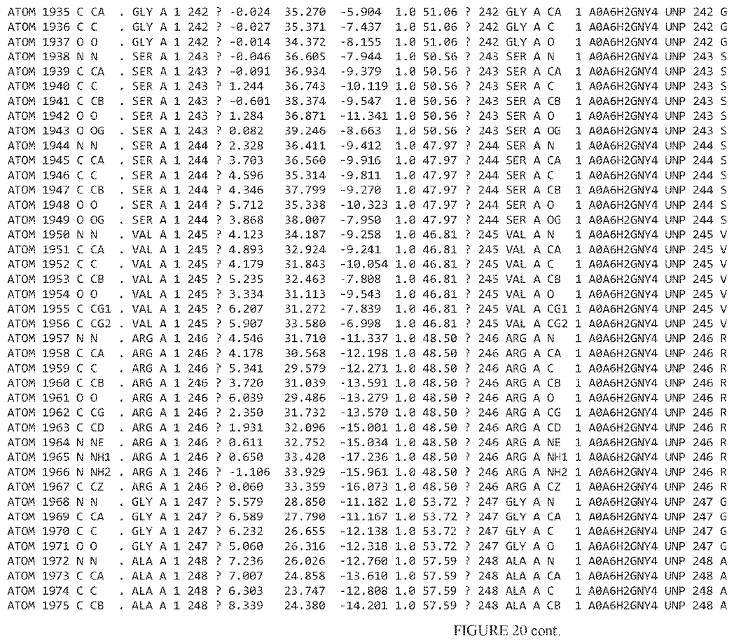

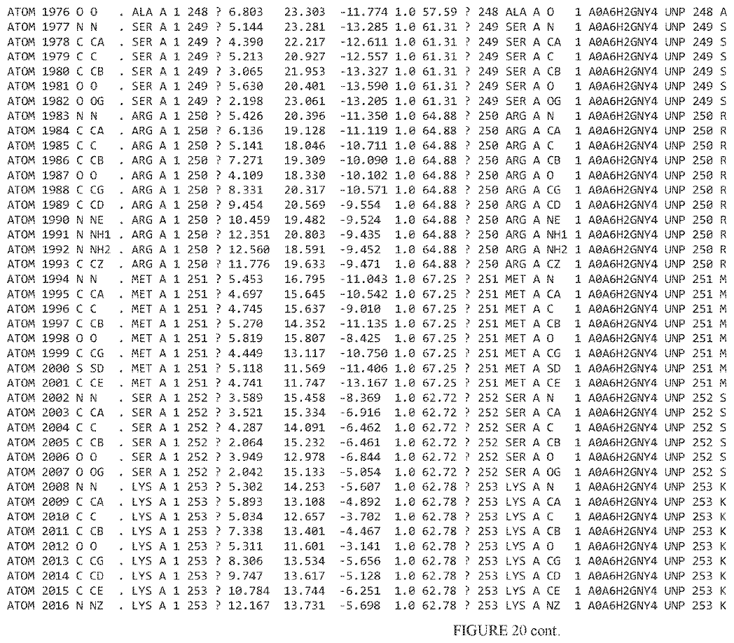

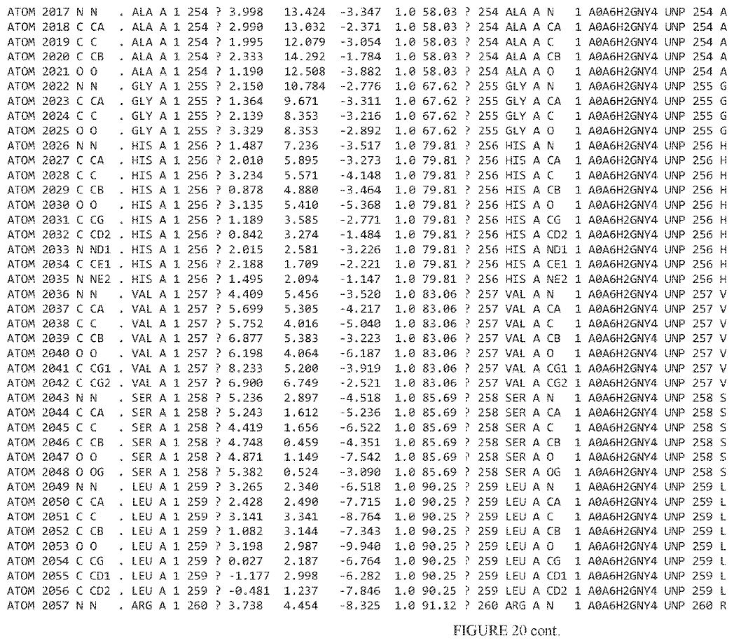

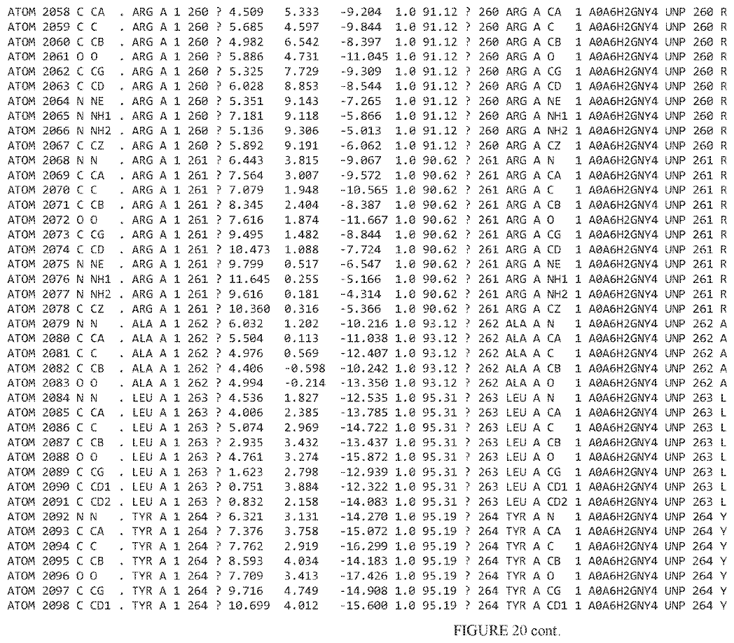

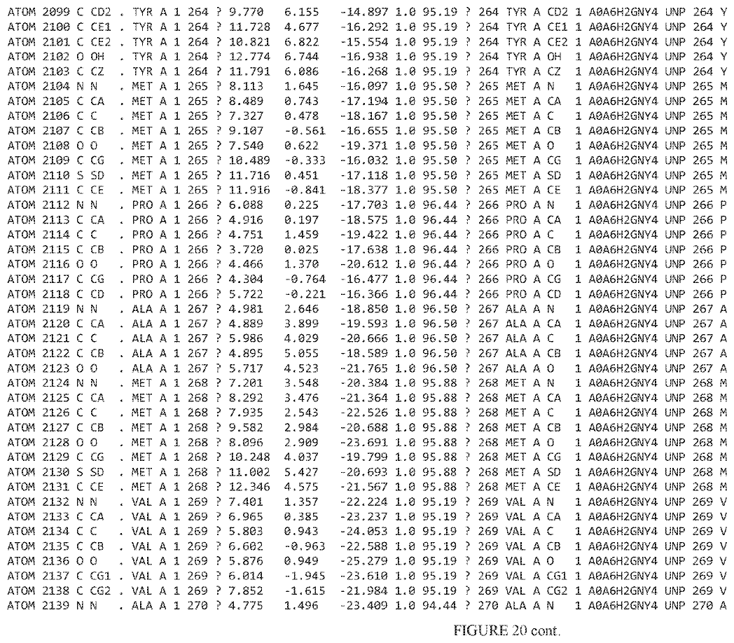

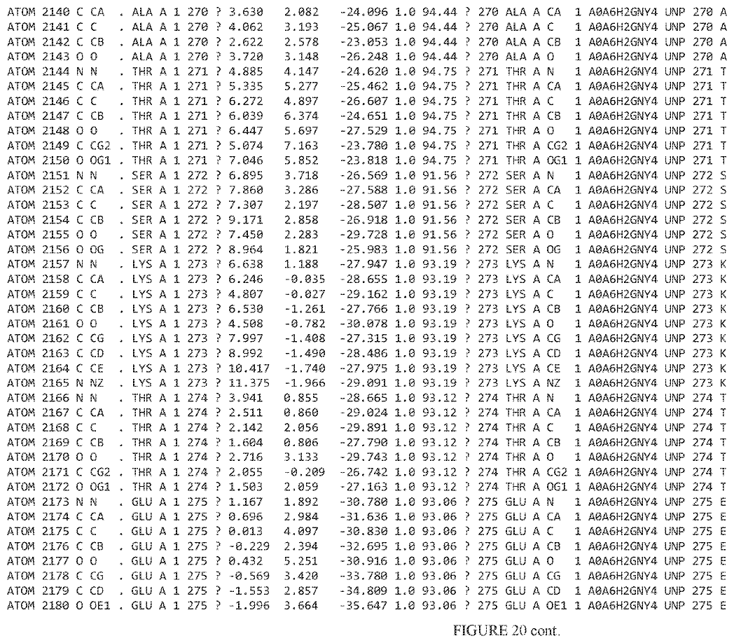

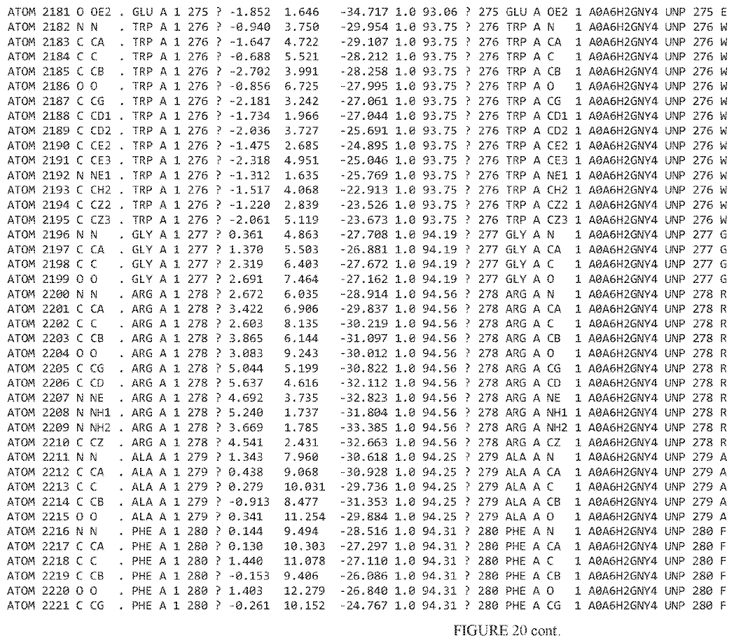

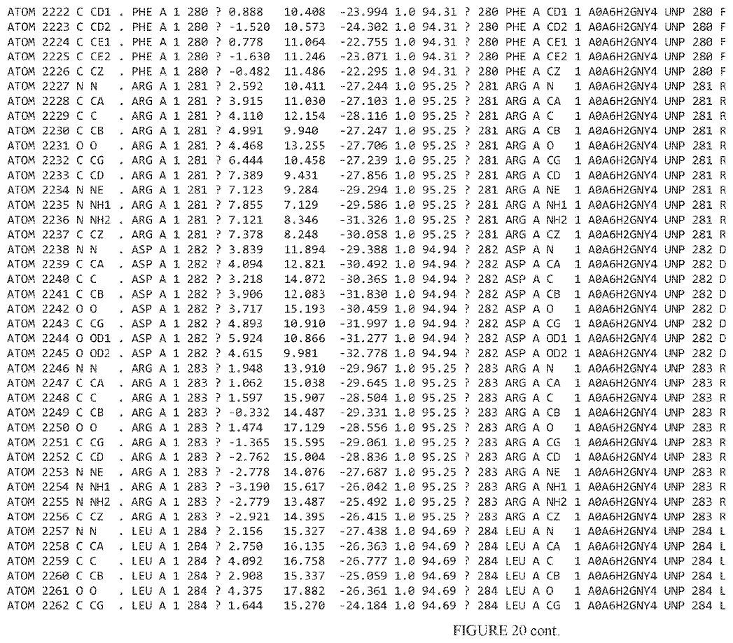

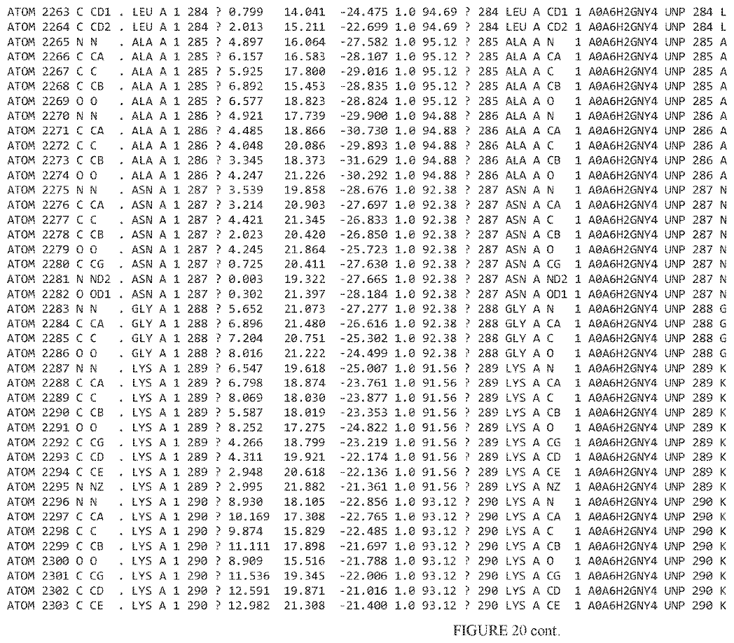

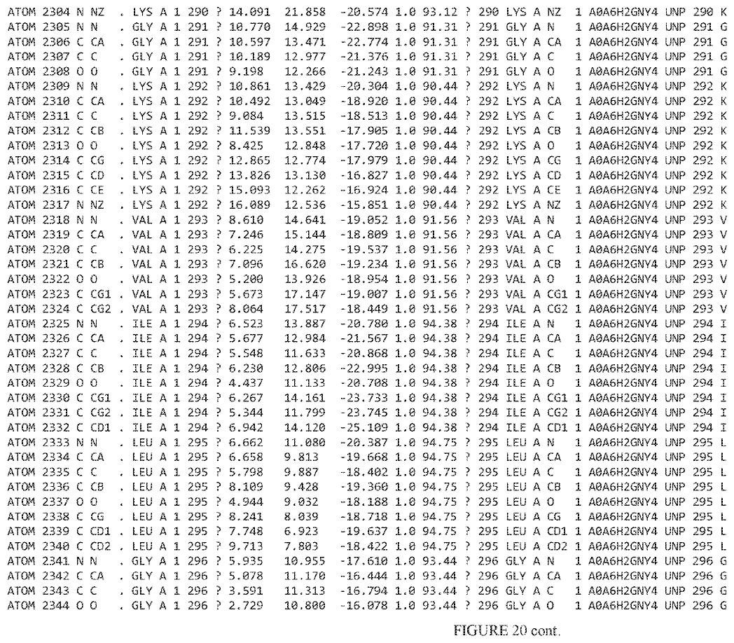

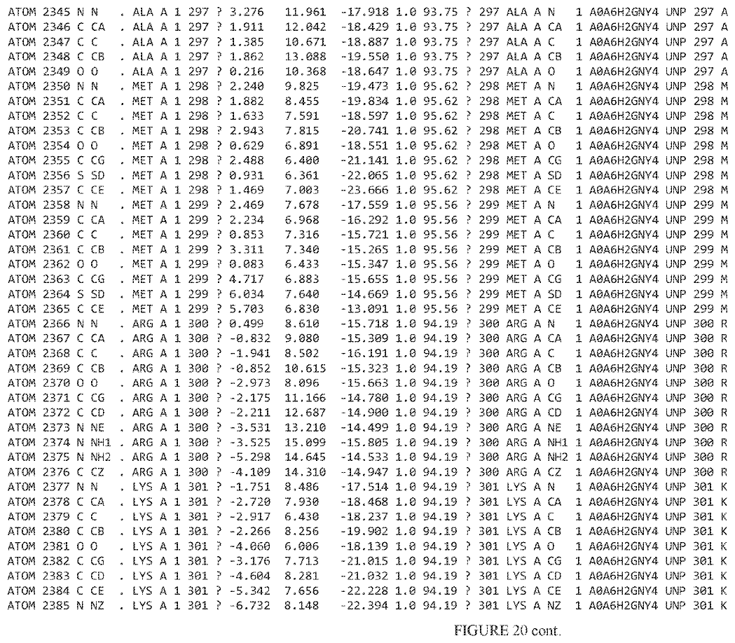

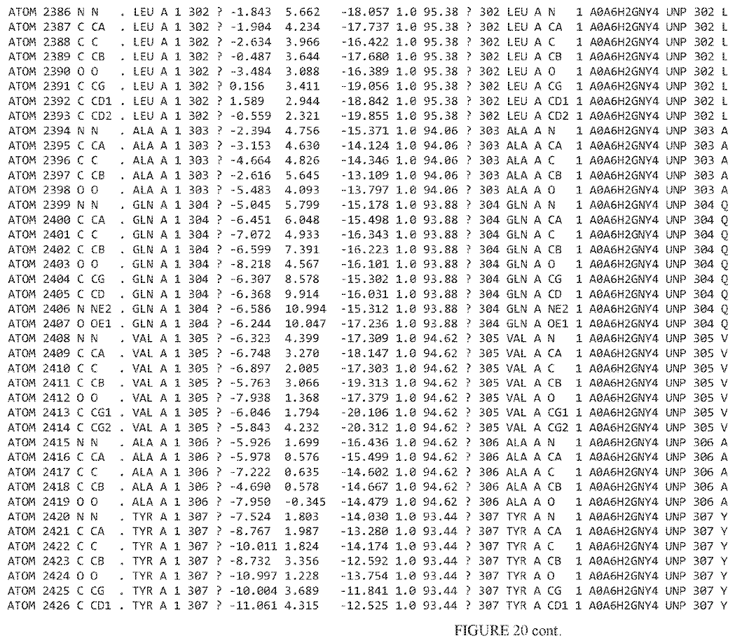

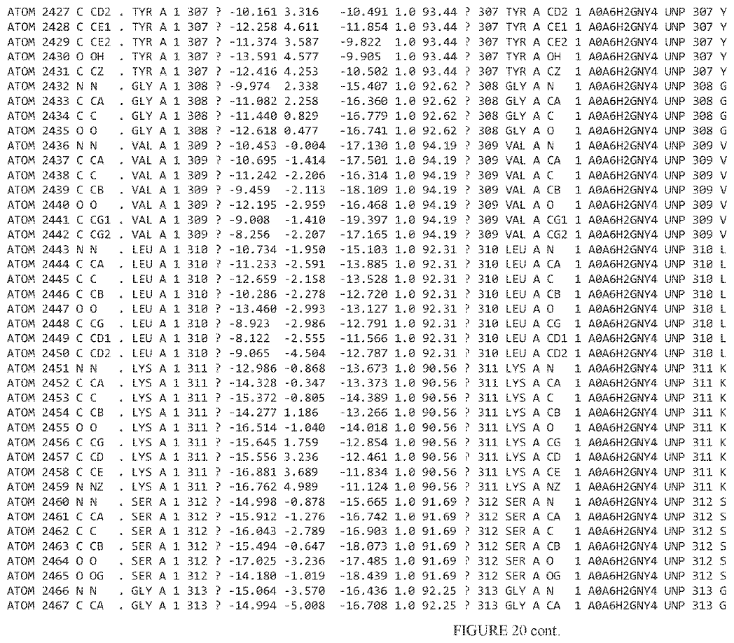

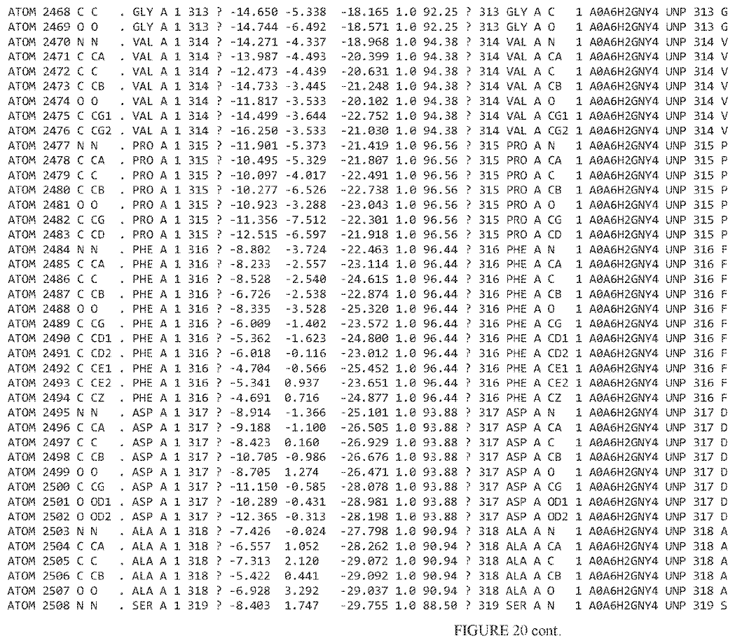

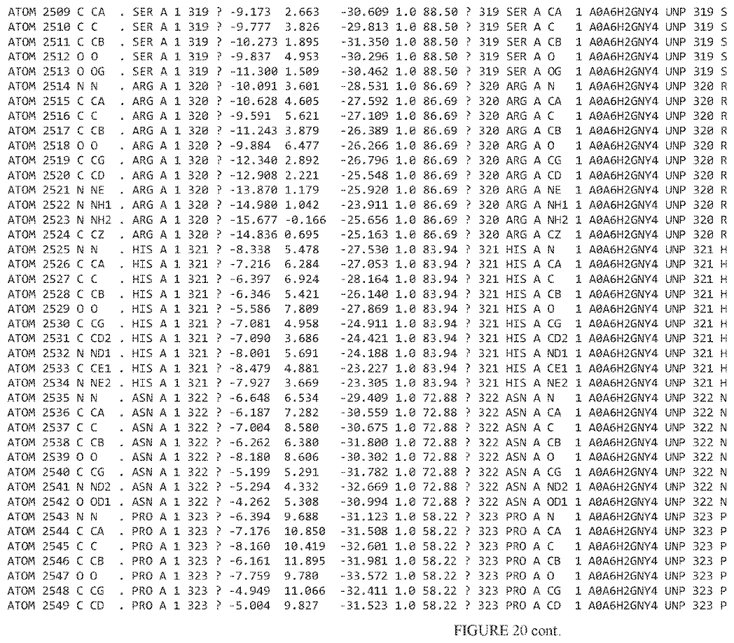

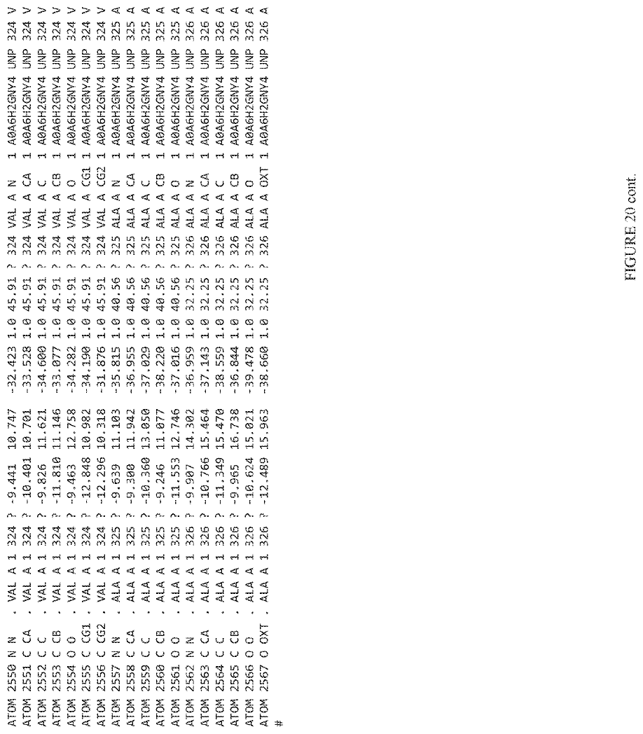

In some embodiments, the bridgeRNA comprises a nucleotide sequence comprising any of the 5′ to 3′ sequences provided in FIG. 19, wherein “n” represents any nucleotide, “R” represents an A or G nucleotide, and “Y” represents a C or U nucleotide. In some embodiments, the bridgeRNA comprises a nucleotide sequence comprising any of the 5′ to 3′ sequences provided in FIG. 19, wherein “n” represents any nucleotide, “R” represents an A or G nucleotide, and “Y” represents a C or U nucleotide and wherein the bridgeRNA comprises the 5′ to 3′ secondary structure provided in the first row of secondary structure for said sequence provided in FIG. 19, wherein matching parentheses “(“and”)” indicate base-paired nucleotides, and “.” indicate unpaired bases. In some embodiments, the bridgeRNA comprises a nucleotide sequence comprising any of the 5′ to 3′ sequences provided in FIG. 19, wherein “n” represents any nucleotide, “R” represents an A or G nucleotide, and “Y” represents a C or U nucleotide and wherein the bridgeRNA comprises the 5′ to 3′ secondary structure provided in the second row of secondary structure for said sequence provided in FIG. 19, wherein matching parentheses “(“and”)” indicate base-paired nucleotides, and “.” indicate unpaired bases. In some embodiments, the bridgeRNA comprises a nucleotide sequence comprising any of the 5′ to 3′ sequences provided in FIG. 19, wherein “n” represents any nucleotide, “R” represents an A or G nucleotide, and “Y” represents a C or U nucleotide and wherein the bridgeRNA comprises the 5′ to 3′ secondary structure provided in the third row of secondary structure for said sequence provided in FIG. 19, wherein matching parentheses “(“and”)” indicate base-paired nucleotides, and “.” indicate unpaired bases. In some embodiments, the bridgeRNA comprises a nucleotide sequence comprising any of the 5′ to 3′ sequences provided in FIG. 19, wherein “n” represents any nucleotide, “R” represents an A or G nucleotide, and “Y” represents a C or U nucleotide and wherein the bridgeRNA comprises the 5′ to 3′ secondary structure provided in the fourth row of secondary structure for said sequence provided in FIG. 19, wherein matching parentheses “(“and”)” indicate base-paired nucleotides, and “.” indicate unpaired bases. In some embodiments, said bridgeRNA binds the IS110 transposase indicated after the “>” for said sequence provided in FIG. 19. In some embodiments, the nucleotide sequence or nucleotide sequence and secondary structure from FIG. 19 are for the bridgeRNAs of ISPa11, IsPa29, ISMmg1, ISPfl1, ISMae40, ISStma6, ISAzs32, ISMex9, ISCARN28, ISAar16, ISCps7, ISPpu9, ISRel9, ISEsa2, ISMma5, IS900, or ISHne5.

The target binding loop of the first stem-loop structure comprises a nucleotide sequence referred to as a left-target guide (LTG) and a nucleotide sequence referred to as a right-target guide (RTG). In some embodiments, the LTG and RTG sequences can be reprogrammed in order to bind to a target site of interest. See Section E. In some embodiments, the LTG and RTG sequences are not reprogrammed and the transpososome comprising such a bridgeRNA will target the wild-type target site sequence and identical sequences found in other organisms, or other sites that are similar to that site (e.g., sequences that are similar or identical to the wild-type target site sequence of the transposase). See Section E.

The donor binding loop of the second stem-loop structure comprises a nucleotide sequence referred to as a left-donor guide (LDG) and a nucleotide sequence referred to as a right-donor guide (RDG). In some embodiments, the LDG and RDG sequences can be reprogrammed in order to bind to a donor site sequence of interest. See Section E. In some embodiments, the LDG and RDG sequences are not reprogrammed and the transpososome comprising such a bridgeRNA will target the wild-type donor sequence and identical sequences found in other organisms, or other sites that are similar to that site (e.g., sequences that are similar or identical to the wild-type donor site sequence of the transposase). See Section E.

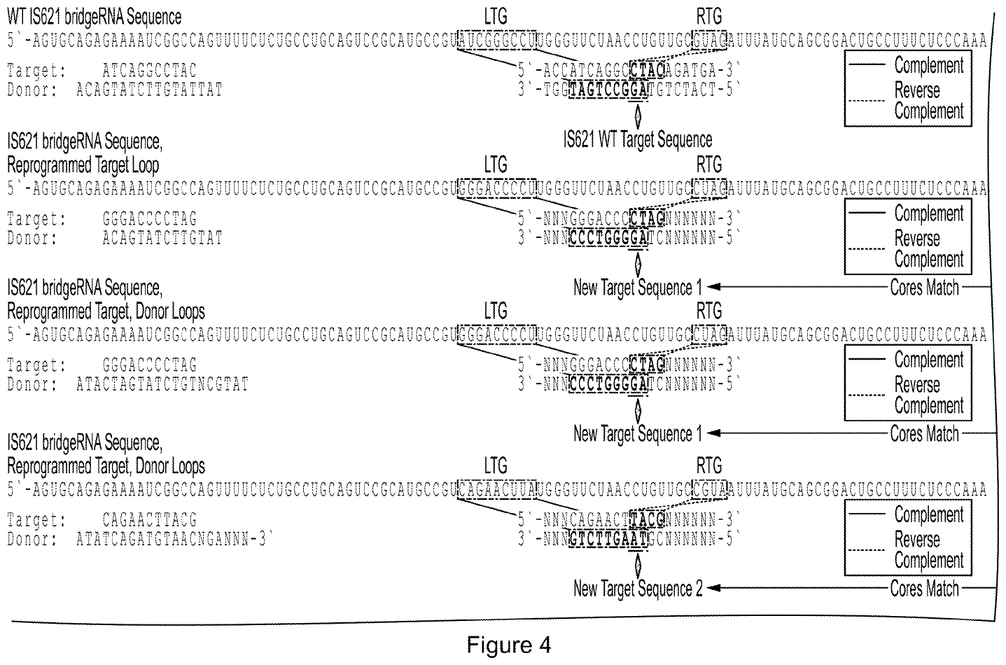

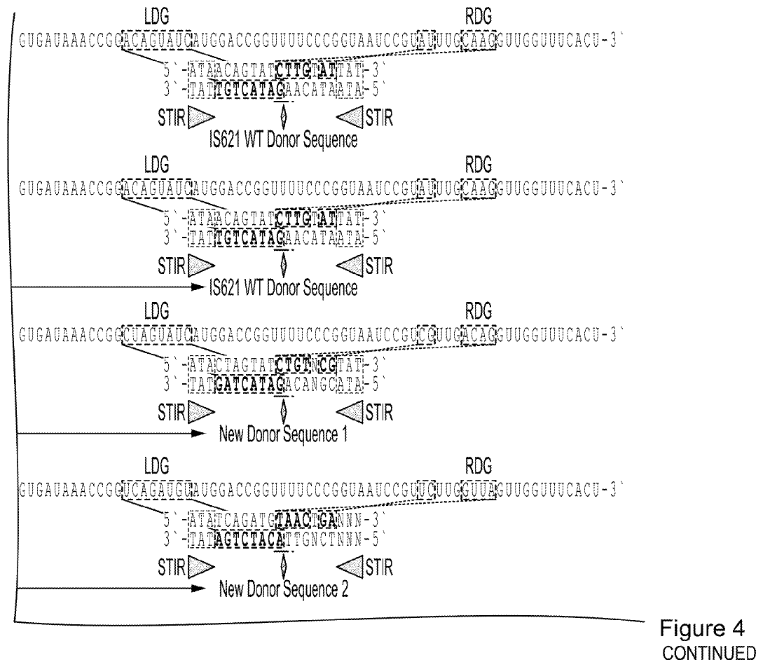

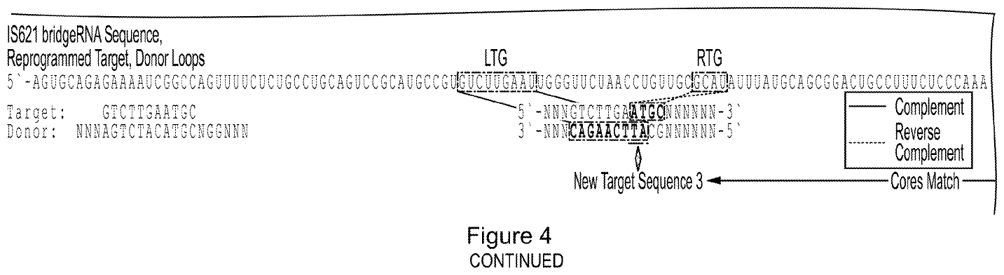

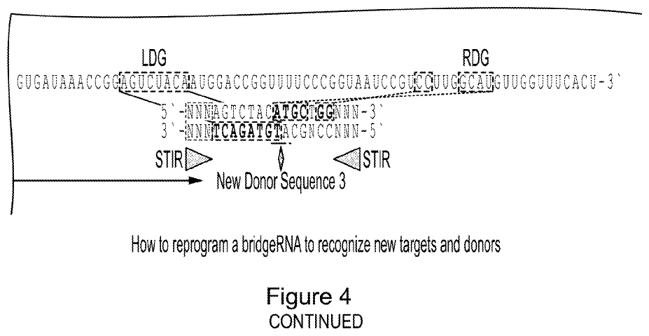

IS110 donor and target site specificity thus may be encoded by the nucleotide sequences of the bridgeRNA found within the target binding and donor binding loops. The sequence of at least one of the LTG, RTG, LDG, or RDG may be reprogrammed (relative to the wild-type IS110 bridgeRNA sequence) via substitutions, insertions, deletions, truncations, and extensions. See FIG. 4 and Section E. Advantageously, described herein is reprogramming of at least one of LTG, RTG, LDG, or RDG can impart specificity for any predefined sequence. BridgeRNA sequences may be reprogrammed via substitutions, insertions, deletions, truncations, and extensions. As described above, certain non-canonical base pairing, mismatch and/or non-contiguous tolerance within the target binding and/or donor binding loops may be acceptable.

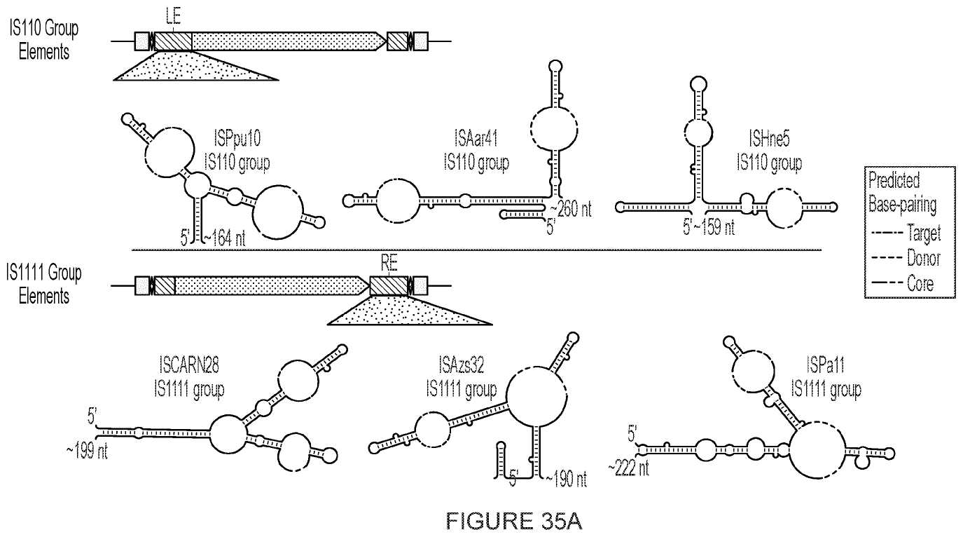

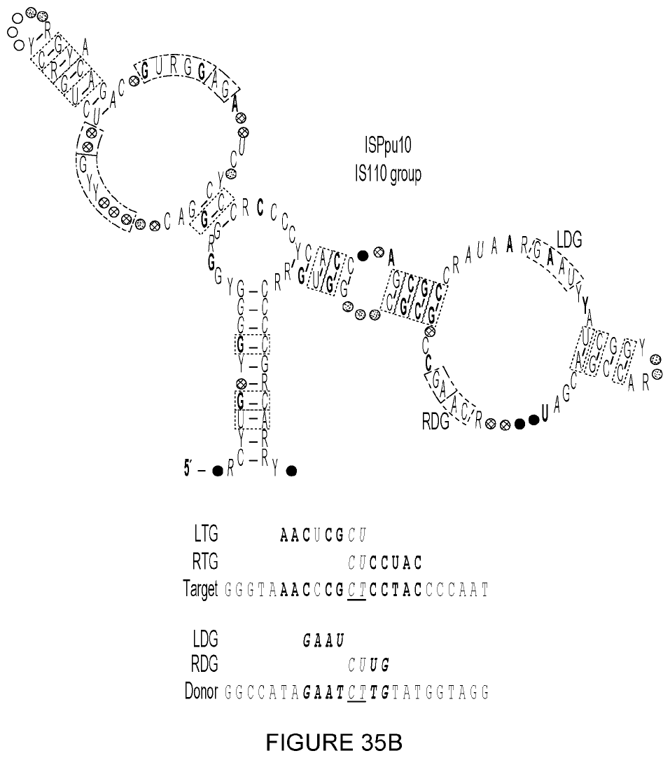

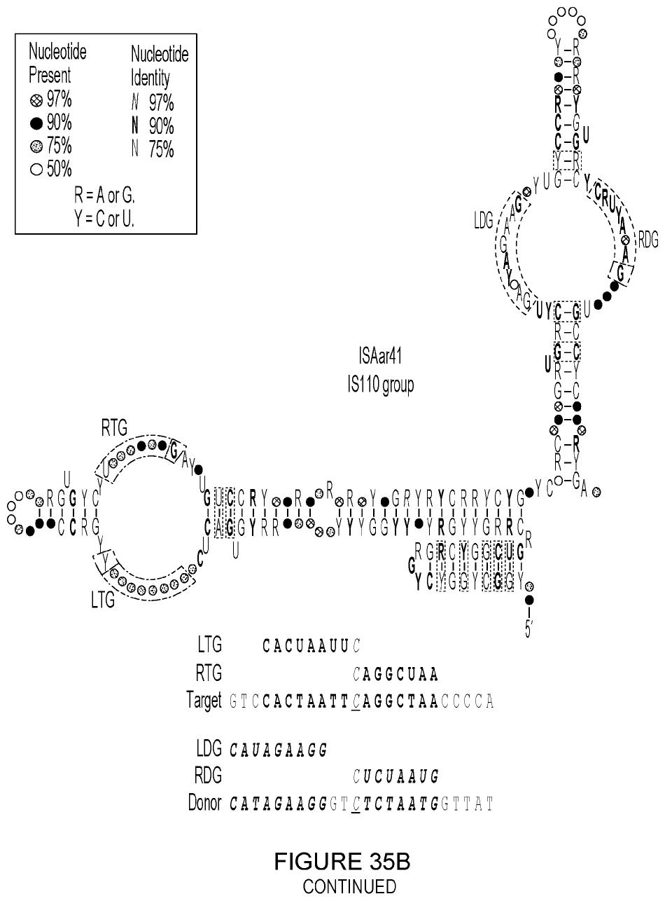

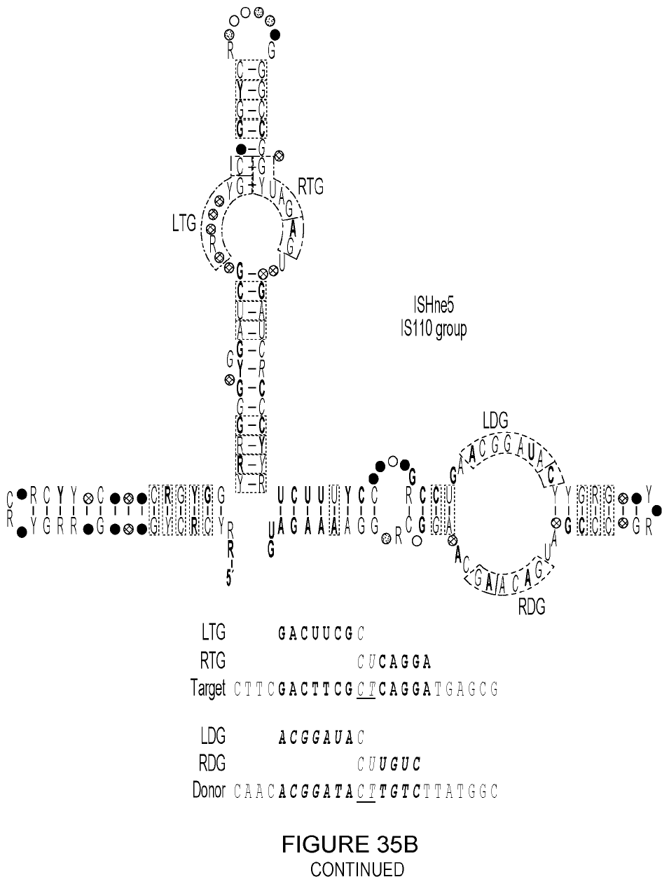

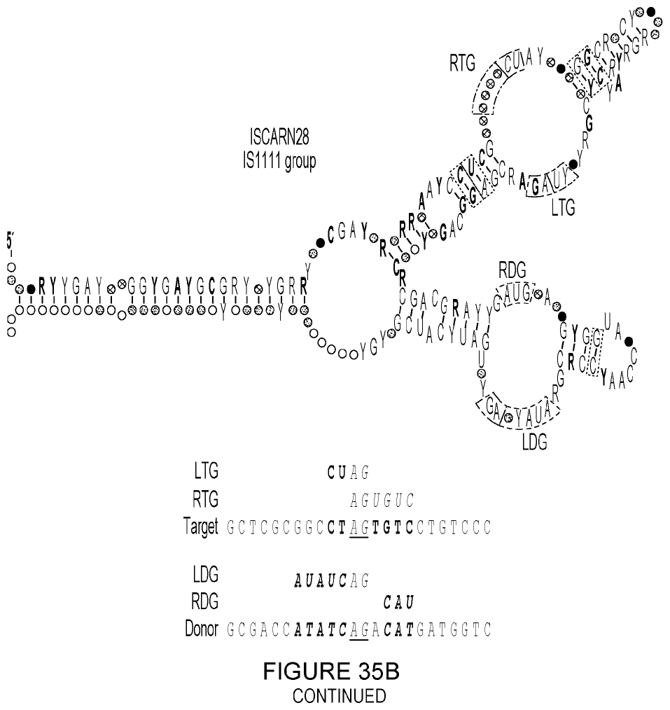

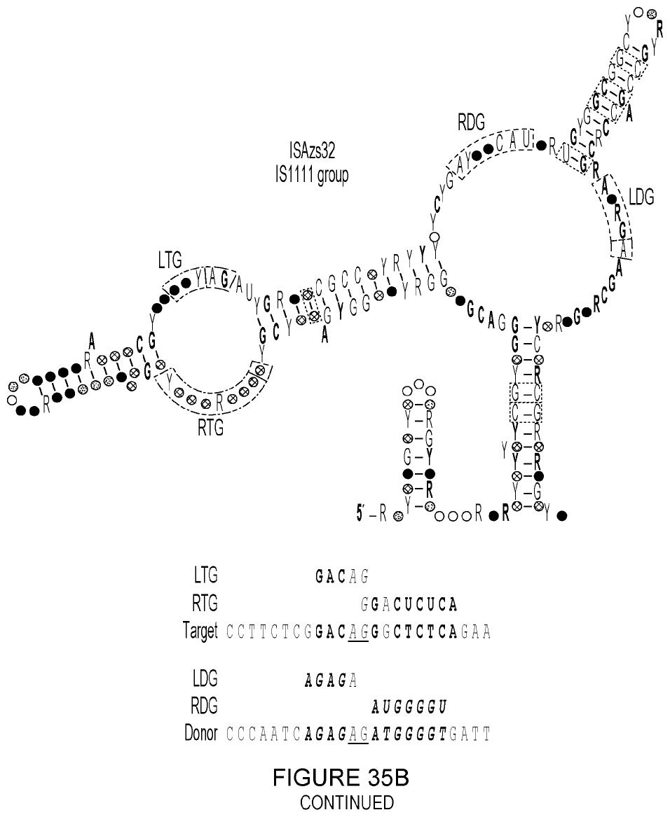

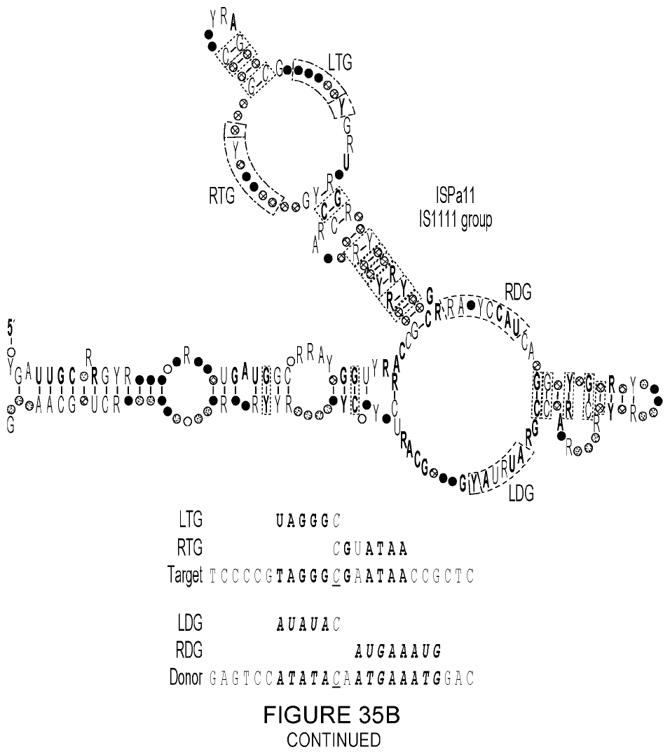

In some embodiments, the bridgeRNA comprises a RNA molecule that comprises a stem-loop structure comprising at least one multi-branched loop comprising at least three stems. In some embodiments, the bridgeRNA comprises at least a first internal loop referred to as a target binding loop and comprising an RTG and LTG sequence and a second internal loop referred to as a donor binding loop comprising an RDG and LDG sequence. In some embodiments, the bridgeRNA comprises a RNA molecule that comprises a stem-loop structure comprising at least one multi-branched loop comprising at least three stems as depicted for Cluster 1 in FIG. 13. In Cluster 1 of FIG. 13 and in some embodiments, the stem-loop structure of the bridgeRNA comprises a multi-branched loop comprising a first stem, a second stem which comprises a stem-loop and an internal loop, and a third stem which comprises a stem-loop. The first stem, second stem, or third stem can include additional loops and bubbles. One or more of the loops (e.g., the multi-branched loop, or one or more internal loops, e.g., the internal loop of the second stem) may correspond to the target binding loop and donor binding loop and comprise nucleotide sequences corresponding to the RTG and LTG sequences and RDG and LDG sequences. In some embodiments, the internal loop of the second stem comprises a target binding loop and the multi-branched loop comprises a donor binding loop. See e.g., FIGS. 35A-B. As shown in Cluster 1 of FIG. 13 and in some embodiments, the bridgeRNA may further comprise an additional stem-loop structure 5′ to the multi-branched loop structure. In some embodiments, said bridgeRNA binds the transposase of ISPa11, ISPa29, ISMmg1, or ISPfl1. In some embodiments, the donor binding loop and/or target binding loops may form a stem or partial stem structure when not bound by a transposase and form a single stranded structure when bound to a transposase.

In some embodiments, the bridgeRNA comprises a RNA molecule that comprises a stem-loop structure comprising at least one multi-branched loop comprising at least three stems. In some embodiments, the bridgeRNA comprises at least a first internal loop referred to as a target binding loop and comprising an RTG and LTG sequence and a second internal loop referred to as a donor binding loop comprising an RDG and LDG sequence. In some embodiments, the bridgeRNA comprises a RNA molecule that comprises a stem-loop structure comprising at least one multi-branched loop comprising at least three stems as depicted for Cluster 3 in FIG. 13. In Cluster 3 of FIG. 13 and in some embodiments, the stem-loop structure of the bridgeRNA comprises a multi-branched loop comprising a first stem, a second stem which comprises a stem-loop and an internal loop, and a third stem which comprises a stem-loop. The first stem, second stem, or third stem can include additional loops and bubbles. One or more of the loops (e.g., the multi-branched loop, or one or more internal loops, e.g., the internal loop of the second stem) may correspond to the target binding loop and donor binding loop and comprise nucleotide sequences corresponding to the RTG and LTG sequences and RDG and LDG sequences. In some embodiments, the internal loop of the second stem comprises a target binding loop and the multi-branched loop comprises a donor binding loop. See e.g., FIGS. 35A-B. As shown in Cluster 3 of FIG. 13, the bridgeRNA further comprises an additional stem-loop structure 5′ to the multi-branched loop structure. In some embodiments, said bridgeRNA binds the transposase of ISAzs32 or ISMex9. In some embodiments, the donor binding loop and/or target binding loops may form a stem or partial stem structure when not bound by a transposase and form a single stranded structure when bound to a transposase.

In some embodiments, the bridgeRNA comprises a RNA molecule that comprises a stem-loop structure comprising at least one multi-branched loop comprising at least three stems. In some embodiments, the bridgeRNA comprises at least a first internal loop referred to as a target binding loop and comprising an RTG and LTG sequence and a second internal loop referred to as a donor binding loop comprising an RDG and LDG sequence. In some embodiments, the bridgeRNA comprises a RNA molecule that comprises a stem-loop structure comprising at least one multi-branched loop comprising at least three stems as depicted for Cluster 4 in FIG. 13. In Cluster 4 of FIG. 13 and in some embodiments, the stem-loop structure of the bridgeRNA comprises a multi-branched loop comprising a first stem, a second stem which comprises a stem-loop and an internal loop, and a third stem which comprises a stem-loop and an internal loop. The first stem, second stem, or third stem can include additional loops and bubbles. One or more of the loops (e.g., the multi-branched loop, or one or more internal loops, e.g., the internal loop of the second stem or internal loop of the third stem) may correspond to the target binding loop and donor binding loop and comprise nucleotide sequences corresponding to the RTG and LTG sequences and RDG and LDG sequences. In some embodiments, the internal loop of the second stem comprises a target binding loop and the internal loop of the third stem comprises a donor binding loop. See e.g., FIGS. 35A-B. In some embodiments, said bridgeRNA binds the transposase of ISCARN28. In some embodiments, the donor binding loop and/or target binding loops may form a stem or partial stem structure when not bound by a transposase and form a single stranded structure when bound to a transposase.

In some embodiments, the bridgeRNA comprises a RNA molecule that comprises a stem-loop structure comprising a clover-leaf like structure comprising at least three stem-loops. In some embodiments, the bridgeRNA comprises at least a first internal loop referred to as a target binding loop and comprising an RTG and LTG sequence and a second internal loop referred to as a donor binding loop comprising an RDG and LDG sequence. In some embodiments, the bridgeRNA comprises a RNA molecule that comprises a stem-loop structure comprising a clover-leaf like structure comprising at least three stem-loops as depicted for Cluster 5 or Cluster 12 in FIG. 13. In Cluster 5 or Cluster 12 of FIG. 13 and in some embodiments, the stem-loop structure of the bridgeRNA comprises a clover-leaf like structure comprising a first stem, a first stem-loop, a second stem-loop which comprises an internal loop, and a third stem-loop which comprises a an internal loop. The stems of the first stem, first stem-loop, second stem-loop, or third stem-loop can include additional loops and bubbles. One or more of the loops (e.g., the one or more internal loops, e.g., the internal loop of the second stem-loop or internal loop of the third stem-loop) may correspond to the target binding loop and donor binding loop and comprise nucleotide sequences corresponding to the RTG and LTG sequences and RDG and LDG sequences. In some embodiments, the internal loop of the second stem-loop comprises a target binding loop and the internal loop of the third stem-loop comprises a donor binding loop. See e.g., FIGS. 35A-B. In some embodiments, said bridgeRNA binds the transposase of ISAar16 or ISHne5. In some embodiments, the donor binding loop and/or target binding loops may form a stem or partial stem structure when not bound by a transposase and form a single stranded structure when bound to a transposase.

In some embodiments, the bridgeRNA comprises a RNA molecule that comprises a stem-loop structure comprising at least one multi-branched loop comprising at least three stems. In some embodiments, the bridgeRNA comprises at least a first internal loop referred to as a target binding loop and comprising an RTG and LTG sequence and a second internal loop referred to as a donor binding loop comprising an RDG and LDG sequence. In some embodiments, the bridgeRNA comprises a RNA molecule that comprises a stem-loop structure comprising at least one multi-branched loop comprising at least three stems as depicted for Cluster 6 in FIG. 13. In Cluster 6 of FIG. 13 and in some embodiments, the stem-loop structure of the bridgeRNA comprises a multi-branched loop comprising a first stem, a second stem which comprises a stem-loop, and a third stem which comprises a stem-loop and an internal loop. The first stem, second stem, or third stem can include additional loops and bubbles. One or more of the loops (e.g., the multi-branched loop, or one or more internal loops, e.g., the internal loop of the third stem) may correspond to the target binding loop and donor binding loop and comprise nucleotide sequences corresponding to the RTG and LTG sequences and RDG and LDG sequences. In some embodiments, the internal loop of the third stem comprises a target binding loop and the multi-branched loop comprises a donor binding loop. In some embodiments, said bridgeRNA binds the transposase of ISCps7. In some embodiments, the donor binding loop and/or target binding loops may form a stem or partial stem structure when not bound by a transposase and form a single stranded structure when bound to a transposase.

In some embodiments, the bridgeRNA comprises a RNA molecule that comprises a stem-loop structure comprising at least one multi-branched loop comprising at least three stems. In some embodiments, the bridgeRNA comprises at least a first internal loop referred to as a target binding loop and comprising an RTG and LTG sequence and a second internal loop referred to as a donor binding loop comprising an RDG and LDG sequence. In some embodiments, the bridgeRNA comprises a RNA molecule that comprises a stem-loop structure comprising at least one multi-branched loop comprising at least three stems as depicted for Cluster 7 in FIG. 13. In Cluster 7 of FIG. 13 and in some embodiments, the stem-loop structure of the bridgeRNA comprises a multi-branched loop comprising a first stem, a second stem which comprises a stem-loop and an internal loop, and a third stem which comprises a stem-loop and an internal loop. The first stem, second stem, or third stem can include additional loops and bubbles. One or more of the loops (e.g., the multi-branched loop, or one or more internal loops, e.g., the internal loop of the second stem or internal loop of the third stem) may correspond to the target binding loop and donor binding loop and comprise nucleotide sequences corresponding to the RTG and LTG sequences and RDG and LDG sequences. In some embodiments, the internal loop of the second stem comprises a target binding loop and the internal loop of the third stem comprises a donor binding loop. See e.g., FIGS. 35A-B. In some embodiments, said bridgeRNA binds the transposase of ISPpu9 or ISPpu10. In some embodiments, the donor binding loop and/or target binding loops may form a stem or partial stem structure when not bound by a transposase and form a single stranded structure when bound to a transposase.

In some embodiments, the bridgeRNA comprises a RNA molecule that comprises a stem-loop structure comprising at least one multi-branched loop comprising at least four stems and at least two stem-loop structures. In some embodiments, the bridgeRNA comprises at least a first internal loop referred to as a target binding loop and comprising an RTG and LTG sequence and a second internal loop referred to as a donor binding loop comprising an RDG and LDG sequence. In some embodiments, the bridgeRNA comprises a RNA molecule that comprises a stem-loop structure comprising at least one multi-branched loop comprising at least four stems and at least two stem-loop structures as depicted for Cluster 8 in FIG. 13. In Cluster 8 of FIG. 13 and in some embodiments, the stem-loop structure of the bridgeRNA comprises a first stem-loop, a second stem-loop comprising an internal loop, and a multi-branched loop comprising a first stem, a second stem which comprises a stem-loop, a third stem which comprises a stem-loop, and a fourth stem which comprises a stem-loop. The first stem-loop, second stem-loop, and stems of the first, second, third, or fourth stem of the multi-branched loop can include additional loops and bubbles. One or more of the loops (e.g., the multi-branched loop, or one or more internal loops, e.g., the internal loop of the second stem-loop) may correspond to the target binding loop and donor binding loop and comprise nucleotide sequences corresponding to the RTG and LTG sequences and RDG and LDG sequences. In some embodiments, the internal loop of the second stem comprises a target binding loop and the multi-branched loop comprises a donor binding loop. In some embodiments, said bridgeRNA binds the transposase of ISRel9. In some embodiments, the donor binding loop and/or target binding loops may form a stem or partial stem structure when not bound by a transposase and form a single stranded structure when bound to a transposase.

In some embodiments, the bridgeRNA comprises a RNA molecule that comprises at least three stem-loop structures. In some embodiments, the bridgeRNA comprises at least a first internal loop referred to as a target binding loop and comprising an RTG and LTG sequence and a second internal loop referred to as a donor binding loop comprising an RDG and LDG sequence. In some embodiments, the bridgeRNA comprises a RNA molecule that comprises a stem-loop structure comprising at least three stem-loop structures as depicted for Cluster 9 or Cluster 11 in FIG. 13. In Cluster 9 or Cluster 11 of FIG. 13 and in some embodiments, the bridgeRNA comprises a first stem-loop, a second stem-loop comprising an internal loop, and a third stem-loop comprising an internal loop. The first stem-loop, second stem-loop, or third stem-loop can include additional loops and bubbles. One or more of the loops (e.g., the internal loop of the second stem-loop and/or internal loop of the third stem-loop) may correspond to the target binding loop and donor binding loop and comprise nucleotide sequences corresponding to the RTG and LTG sequences and RDG and LDG sequences. In some embodiments, the internal loop of the second stem-loop comprises a target binding loop and the internal loop of the third stem-loop comprises a donor binding loop. In some embodiments, said bridgeRNA binds the transposase of ISEsa2 or IS900. In some embodiments, the donor binding loop and/or target binding loops may form a stem or partial stem structure when not bound by a transposase and form a single stranded structure when bound to a transposase.

In some embodiments, the bridgeRNA comprises a RNA molecule that comprises a stem-loop structure comprising at least one multi-branched loop comprising at least three stems. In some embodiments, the bridgeRNA comprises at least a first internal loop referred to as a target binding loop and comprising an RTG and LTG sequence and a second internal loop referred to as a donor binding loop comprising an RDG and LDG sequence. In some embodiments, the bridgeRNA comprises a RNA molecule that comprises a stem-loop structure comprising at least one multi-branched loop comprising at least three stems as depicted for Cluster 10 in FIG. 13. In Cluster 10 of FIG. 13 and in some embodiments, the stem-loop structure of the bridgeRNA comprises a multi-branched loop comprising a first stem, a second stem which comprises a stem-loop and an internal loop, and a third stem which comprises a stem-loop and an internal loop. The first stem, second stem, or third stem can include additional loops and bubbles. One or more of the loops (e.g., the multi-branched loop, or one or more internal loops, e.g., the internal loop of the second stem or internal loop of the third stem) may correspond to the target binding loop and donor binding loop and comprise nucleotide sequences corresponding to the RTG and LTG sequences and RDG and LDG sequences. In some embodiments, the internal loop of the second stem comprises a target binding loop and the internal loop of the third stem comprises a donor binding loop. In some embodiments, the internal loop of the second stem comprises a target binding loop and the multi-branched loop comprises a donor binding loop. In some embodiments, said bridgeRNA binds the transposase of ISMma5. In some embodiments, the donor binding loop and/or target binding loops may form a stem or partial stem structure when not bound by a transposase and form a single stranded structure when bound to a transposase.

With reference to any of the above bridgeRNAs, in some embodiments, the LTG and RTG sequences are fully complementary to their respective target site sequences of the target DNA. In some embodiments, the LTG and/or RTG are partially complementary to their respective target site sequences of the target DNA, i.e., there is non-canonical base pairing, mismatch and/or non-contiguous tolerance. In some embodiments, there is a single mismatch in the LTG or RTG nucleotide sequence. In some embodiments, there are two, three or four mismatches which can be in the LTG, in the RTG, or spread across the LTG and RTG. In some embodiments, the two, three or four mismatches are contiguous. In some embodiments, the two, three or four mismatches are non-contiguous. In some embodiments, there is a single non-canonical base pair in the LTG or RTG nucleotide sequence. In some embodiments, there are two, three or four non-canonical base pairs which can be in the LTG, in the RTG, or spread across the LTG and RTG. In some embodiments, the two, three or four non-canonical base pairs are contiguous. In some embodiments, the two, three or four non-canonical base pairs are non-contiguous. In some embodiments, the LDG and RDG nucleotide sequences are fully complementary to their respective donor site sequences of the donor DNA. In some embodiments, the LDG and/or RDG nucleotide sequences are partially complementary to their respective donor site sequences of the donor DNA, i.e., there is non-canonical base pairing, mismatch and/or non-contiguous tolerance. In some embodiments, there is a single mismatch in the LDG or RDG nucleotide sequence. In some embodiments, there are two, three, or four mismatches which can be in the LDG, in the RDG, or spread across the LDG and RDG. In some embodiments, the two, three, or four mismatches are contiguous. In some embodiments, the two, three, or four mismatches are non-contiguous. In some embodiments, there is a single non-canonical base pair in the LDG or RDG nucleotide sequence. In some embodiments, there are two, three, or four non-canonical base pairs which can be in the LDG, in the RDG, or spread across the LDG and RDG. In some embodiments, the two, three, or four non-canonical base pairs are contiguous. In some embodiments, the two, three, or four non-canonical base pairs are non-contiguous. In some embodiments, the non-canonical base pairing is non-Watson-Crick base pairing (i.e., is not a G-C base pair or A-T/U base pair). In some embodiments, the non-canonical base pairing is wobble base pairing. In some embodiments, the non-canonical base pairing is Hoogsteen base pairing. In some embodiments, the non-canonical base pairing comprises a rG-dT base pair, a rU-dG base pair, a rA-dC base pair, a rC-dA base pair, a rA-dG base pair, or a rG-dG base pair.

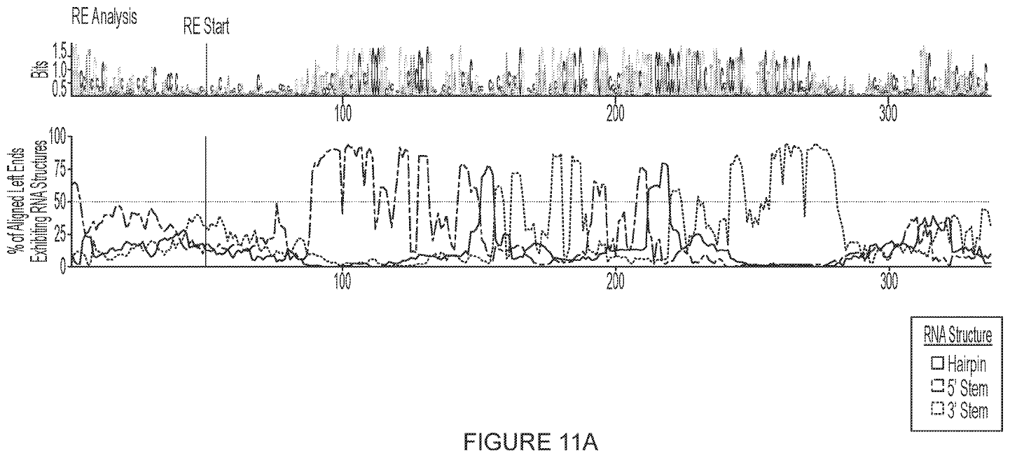

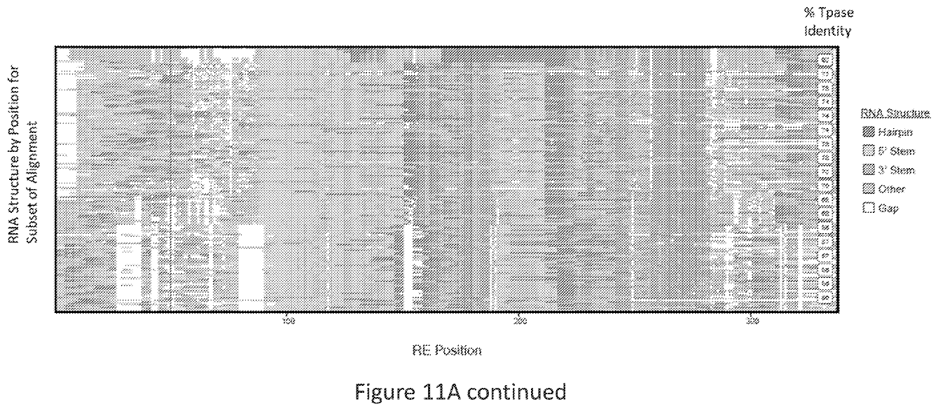

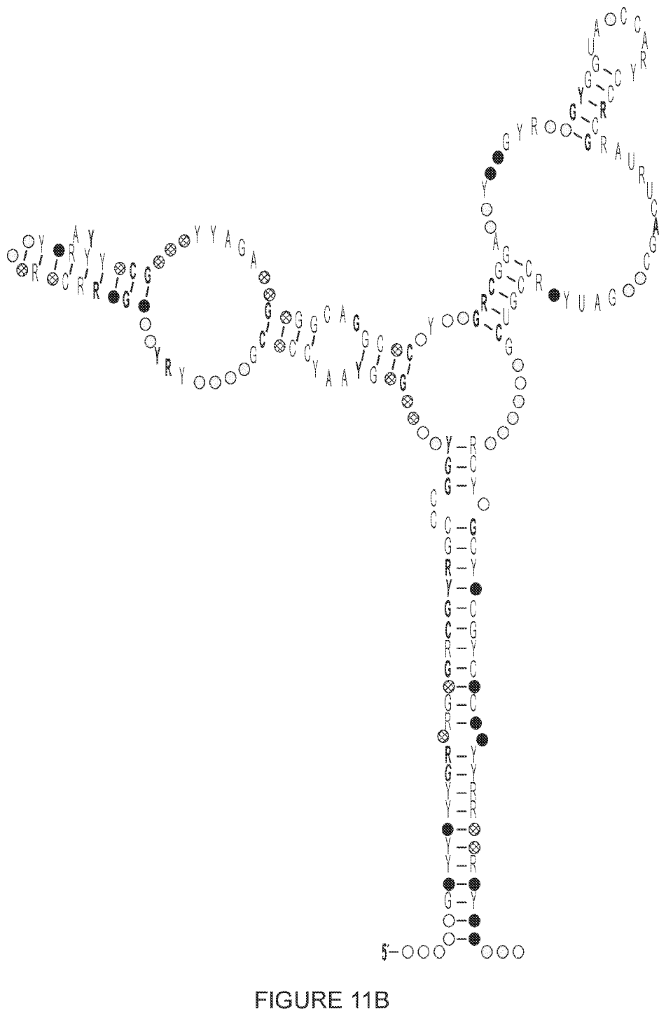

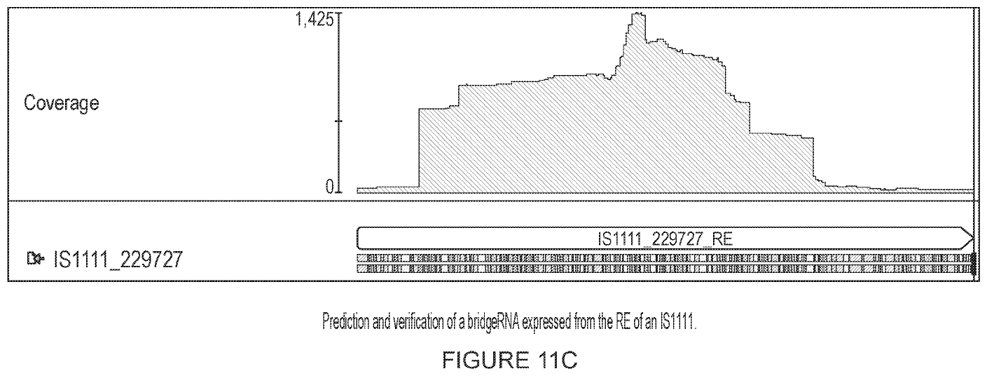

In some embodiments, the bridgeRNA comprises a RNA molecule that comprises a stem-loop structure comprising at least one multi-branched loop comprising at least three stems. In some embodiments, the bridgeRNA comprises at least a first internal loop referred to as a target binding loop and comprising an RTG and LTG sequence and a second internal loop referred to as a donor binding loop comprising an RDG and LDG sequence. In some embodiments, the bridgeRNA comprises a RNA molecule that comprises a stem-loop structure comprising at least one multi-branched loop comprising at least three stems as depicted for IS1111 transposases in FIG. 11B. In FIG. 11B and in some embodiments, the stem-loop structure of the bridgeRNA comprises a multi-branched loop comprising a first stem, a second stem which comprises a stem-loop and an internal loop, and a third stem which comprises a stem-loop and an internal loop. The first stem, second stem, or third stem can include additional loops and bubbles. One or more of the loops (e.g., the internal loop of the second stem and internal loop of the third stem) may correspond to the target binding loop and donor binding loop and comprise nucleotide sequences corresponding to the RTG and LTG sequences and RDG and LDG sequences. In some embodiments, the internal loop of the second stem comprises a target binding loop and the internal loop of the third stem comprises a donor binding loop. In some embodiments, said bridgeRNA binds the transposase of IS1111_229727. In some embodiments, the donor binding loop and/or target binding loops may form a stem or partial stem structure when not bound by a transposase and form a single stranded structure when bound to a transposase.

Thus, in some embodiments, the bridgeRNA comprises a nucleotide sequence comprising the following secondary structure: first stem-multi-branched loop-second stem comprising a stem-loop and an internal loop-third stem comprising a stem-loop and internal loop. In some embodiments, the bridgeRNA comprises additional stem-loop structures, bulges, and/or loops (see, e.g., FIG. 11B). In some embodiments, the bridgeRNA comprises a nucleotide sequence comprising 5′-[A]-[B]-[C]-[D]-[E]-[F]-[G]-[H]-[I]-[J]-[K]-[L]-[M]-[N]—[O]—[P]-[Q]-[R]—[S]-3′, wherein A is a first stem portion, B is a first portion of a multi-branched loop, C is second stem portion, D is a first side of an internal loop corresponding to a target binding loop, E is a third stem portion, F is a first loop portion, G is the reverse complement of E, H is a second side of the internal loop corresponding to the target binding loop, I is the reverse complement of C, J is a second portion of the multi-branched loop, K is a fourth stem portion, L is a first side of an internal loop corresponding to a donor binding loop, M is a fifth stem portion, N is a second loop portion, O is the reverse complement of M, P is a second side of the internal loop corresponding to the donor binding loop, Q is the reverse complement of K, R is a third portion of the multi-branched loop, and S is the reverse complement of A. In some embodiments, the reverse complement portions are not 100% complementary so that the stem structures may comprise one or more mismatches or bulges, or non-standard base-pairing may occur. In some embodiments, the first side of the internal loop corresponding to the target binding loop comprises a nucleotide sequence that is complementary to a first target site sequence of a target DNA (referred to as LTG) and the second side of the internal loop corresponding to the target binding loop comprises a second nucleotide sequence that is complementary to a second target site sequence which is on the opposite strand of the target DNA to the first target site sequence (referred to as RTG). In some embodiments, the first side of the internal loop corresponding to the donor binding loop comprises a nucleotide sequence that is complementary to a first donor site sequence of a donor DNA (referred to as LDG) and the second side of the internal loop corresponding to the donor binding loop comprises a second nucleotide sequence that is complementary to a second donor site sequence which is on the opposite strand of the donor DNA to the first target site sequence (referred to as RDG). In some embodiments, the stem structures may comprise one or more mismatches or bulges. In some embodiments, at least two portions of nucleotide sequence S are not complementary to the nucleotide sequence of portion A, so that the stem structure formed by base pairing between portion A and S comprises two bulges. In some embodiments, at least one portion of nucleotide sequence I is not complementary to the nucleotide sequence of portion C, so that the stem structure formed by base pairing between portion I and C comprises a bulge. In another embodiment, one or more nucleotides are present between the 5′ terminus and portion A. In another embodiment, one or more nucleotides are present between the 3′ terminus and portion S. In some embodiments, said bridgeRNA binds the transposase of IS1111_229727. In some embodiments, the donor binding loop and/or target binding loops may form a stem or partial stem structure when not bound by a transposase and form a single stranded structure when bound to a transposase.

Thus, in some embodiments, the bridgeRNA comprises a nucleotide sequence comprising the following secondary structure: first stem-multi-branched loop-second stem comprising a stem-loop and an internal loop-third stem comprising a stem-loop and internal loop. In some embodiments, the bridgeRNA comprises additional stem-loop structures, bulges, and/or loops (see, e.g., FIG. 11B). In some embodiments, the bridgeRNA comprises a nucleotide sequence comprising 5′-[A]-[B]-[C]-[D]-[E]-[F]-[G]-[H]-[I]-[J]-[K]-[L]-[M]-[N]—[O]—[P]-[Q]-[R]—[S]-3′, wherein A is a first stem portion, B is a first portion of a multi-branched loop, C is second stem portion, D is a first side of an internal loop corresponding to a donor binding loop, E is a third stem portion, F is a first loop portion, G is the reverse complement of E, H is a second side of the internal loop corresponding to the donor binding loop, I is the reverse complement of C, J is a second portion of the multi-branched loop, K is a fourth stem portion, L is a first side of an internal loop corresponding to a target binding loop, M is a fifth stem portion, N is a second loop portion, O is the reverse complement of M, P is a second side of the internal loop corresponding to the target binding loop, Q is the reverse complement of K, R is a third portion of the multi-branched loop, and S is the reverse complement of A. In some embodiments, the reverse complement portions are not 100% complementary so that the stem structures may comprise one or more mismatches or bulges, or non-standard base-pairing may occur. In some embodiments, the first side of the internal loop corresponding to the target binding loop comprises a nucleotide sequence that is complementary to a first target site sequence of a target DNA (referred to as LTG) and the second side of the internal loop corresponding to the target binding loop comprises a second nucleotide sequence that is complementary to a second target site sequence which is on the opposite strand of the target DNA to the first target site sequence (referred to as RTG). In some embodiments, the first side of the internal loop corresponding to the donor binding loop comprises a nucleotide sequence that is complementary to a first donor site sequence of a donor DNA (referred to as LDG) and the second side of the internal loop corresponding to the donor binding loop comprises a second nucleotide sequence that is complementary to a second donor site sequence which is on the opposite strand of the donor DNA to the first target site sequence (referred to as RDG). In some embodiments, the stem structures may comprise one or more mismatches or bulges. In some embodiments, at least two portions of nucleotide sequence S are not complementary to the nucleotide sequence of portion A, so that the stem structure formed by base pairing between portion A and S comprises two bulges. In some embodiments, at least one portion of nucleotide sequence I is not complementary to the nucleotide sequence of portion C, so that the stem structure formed by base pairing between portion I and C comprises a bulge. In another embodiment, one or more nucleotides are present between the 5′ terminus and portion A. In another embodiment, one or more nucleotides are present between the 3′ terminus and portion S. In some embodiments, said bridgeRNA binds the transposase of IS1111_229727. In some embodiments, the donor binding loop and/or target binding loops may form a stem or partial stem structure when not bound by a transposase and form a single stranded structure when bound to a transposase.

With reference to the above bridgeRNAs, in some embodiments, the LTG and RTG sequences are fully complementary to their respective target site sequences of the target DNA. In some embodiments, the LTG and/or RTG are partially complementary to their respective target site sequences of the target DNA, i.e., there is non-canonical base pairing, mismatch and/or non-contiguous tolerance. In some embodiments, there is a single mismatch in the LTG or RTG nucleotide sequence. In some embodiments, there are two, three or four mismatches which can be in the LTG, in the RTG, or spread across the LTG and RTG. In some embodiments, the two, three or four mismatches are contiguous. In some embodiments, the two, three or four mismatches are non-contiguous. In some embodiments, there is a single non-canonical base pair in the LTG or RTG nucleotide sequence. In some embodiments, there are two, three or four non-canonical base pairs which can be in the LTG, in the RTG, or spread across the LTG and RTG. In some embodiments, the two, three or four non-canonical base pairs are contiguous. In some embodiments, the two, three or four non-canonical base pairs are non-contiguous. In some embodiments, the LDG and RDG nucleotide sequences are fully complementary to their respective donor site sequences of the donor DNA. In some embodiments, the LDG and/or RDG nucleotide sequences are partially complementary to their respective donor site sequences of the donor DNA, i.e., there is non-canonical base pairing, mismatch and/or non-contiguous tolerance. In some embodiments, there is a single mismatch in the LDG or RDG nucleotide sequence. In some embodiments, there are two, three, or four mismatches which can be in the LDG, in the RDG, or spread across the LDG and RDG. In some embodiments, the two, three, or four mismatches are contiguous. In some embodiments, the two mismatches are non-contiguous. In some embodiments, there is a single non-canonical base pair in the LDG or RDG nucleotide sequence. In some embodiments, there are two, three, or four non-canonical base pairs which can be in the LDG, in the RDG, or spread across the LDG and RDG. In some embodiments, the two, three, or four non-canonical base pairs are contiguous. In some embodiments, the two, three, or four non-canonical base pairs are non-contiguous. In some embodiments, the non-canonical base pairing is non-Watson-Crick base pairing (i.e., is not a G-C base pair or A-T/U base pair). In some embodiments, the non-canonical base pairing is wobble base pairing. In some embodiments, the non-canonical base pairing is Hoogsteen base pairing. In some embodiments, the non-canonical base pairing comprises a rG-dT base pair, a rU-dG base pair, a rA-dC base pair, a rC-dA base pair, a rA-dG base pair, or a rG-dG base pair.

In certain aspects, described herein is bridgeRNA comprising means for directing the IS110 transposase to a polynucleotide comprising a donor or target site sequence. In some embodiments, the means for directing the IS110 transposase to a polynucleotide comprising a donor or target site sequence comprises a bridgeRNA sequence of FIG. 15 (SEQ ID NOS: 1-348) or SEQ ID NOS: 349-10175.

BridgeRNA sequence modification may be used to alter binding specificity between a bridgeRNA and an IS110 transposase. For example, bridgeRNA sequence modification may be used to increase affinity of the bridgeRNA for an IS110 transposase or IS110 transpososome.

In some embodiments, the one or more bridgeRNAs are transcribed from the non-coding ends of the IS110 elements, for example from an integrated element (core (if present)-LE-Transposase-RE-core (if present)), or the circular form of the IS110 element (Transposase-RE-core (if present)-LE). In some embodiments, one or more bridgeRNAs are transcribed from an engineered construct derived from the IS110 element non-coding ends. In some embodiments, one or more bridgeRNAs are transcribed from an engineered construct derived from the IS110 element non-coding ends and a 5′ or 3′ portion of the CDS. In some embodiments, one or more bridgeRNAs are transcribed from a nucleotide sequence comprising RE-core-LE. In some embodiments, one or more bridgeRNAs are transcribed from a nucleotide sequence comprising RE-core-LE, wherein RE-core-LE comprises an LE, core, and RE provided in FIG. 15 (SEQ ID NOS: 1-348), FIG. 17 (SEQ ID NOS: 30354-30529), or SEQ ID NOS: 349-10175 or 30530-40356. In some embodiments, one or more bridgeRNAs are transcribed from a nucleotide sequence comprising a portion of an RE-core-LE sequence. In some embodiments, one or more bridgeRNAs are transcribed from a nucleotide sequence comprising RE-LE. In some embodiments, one or more bridgeRNAs are transcribed from a nucleotide sequence comprising RE-LE, wherein RE-LE comprises an LE and RE provided in FIG. 15 (SEQ ID NOS: 1-348), FIG. 17 (SEQ ID NOS: 30354-30529), or SEQ ID NOS: 349-10175 or 30530-40356. In some embodiments, one or more bridgeRNAs are transcribed from a nucleotide sequence comprising a portion of an RE-LE sequence. In some embodiments, one or more bridgeRNAs are transcribed from a nucleotide sequence comprising LE. In some embodiments, LE comprises an LE provided in FIG. 15 (SEQ ID NOS: 1-348), FIG. 17 (SEQ ID NOS: 30354-30529), or SEQ ID NOS: 349-10175 or 30530-40356. In some embodiments, one or more bridgeRNAs are transcribed from a nucleotide sequence comprising a portion of an LE sequence. In some embodiments, one or more bridgeRNAs are transcribed from a nucleotide sequence comprising RE. In some embodiments, RE comprises an RE provided in FIG. 15 (SEQ ID NOS: 1-348), FIG. 17 (SEQ ID NOS: 30354-30529), or SEQ ID NOS: 349-10175 or 30530-40356. In some embodiments, one or more bridgeRNAs are transcribed from a nucleotide sequence comprising a portion of an RE sequence.

Any bridgeRNA described herein may comprise additional nucleotide portions, nucleotide linkers, loops, and modifications (e.g. structured RNA pseudoknots or other RNA structures) as is understood by a person skilled in the art.

Any bridgeRNA described herein may be expressed from linear or circular dsDNA or ssDNA or supplied as a synthetic RNA. When the bridgeRNA is encoded by an expression construct, the bridgeRNA coding sequence can be expressed from a suitable promoter, such as promoter derived from the non-coding ends of an IS110 member or from an ectopic promoter, such as a type III pol III promoter.

In one embodiment, the present invention contemplates an approach to engineering IS110 systems. Given the abundance and diversity of IS110 orthologs, taking different domains or sequences from closely or distantly related orthologs and creating new combinations could yield new IS110 systems with favorable enzymatic properties. In one embodiment, one IS110 ortholog may have a particular RuvC-like DEDD catalytic domain with high efficiency, and another IS110 may have a bridgeRNA with highly specific target and/or donor loops. These IS110 systems could be combined in such a way that takes advantage of the favorable properties of each. In another embodiment, the bridgeRNA binding subdomains of the RuvC-like DEDD catalytic domain and transposase domains may have an affinity for a particular bridgeRNA, and they may be swapped and combined with a related system to take advantage of the favorable properties of each. In another embodiment, two or more distinct bridgeRNA structures could be compared, and their subsequences and domains could be swapped to yield new bridgeRNA species with favorable properties.

For example, bridgeRNA structural models could be built using standard bioinformatic software packages such as Infernal (Nawrocki and Eddy 2013). These models could be searched against genomic sequence databases to identify bridgeRNA sequences, and the length and sequence of the guide subsequences of the predicted target and donor site binding loops for each candidate could be calculated. Candidate IS110 systems with especially long guides encoded within the target and/or donor binding loops could be prioritized for experimental characterization. In another embodiment, target loop sequences could be searched against nearby flanking sequences to identify complementary target sequences, and candidates with especially long matches between target sites and target binding loops could be prioritized for experimental characterization. In another embodiment, bridgeRNA donor binding loop sequences could be searched against IS110 terminal end sequences to identify complementary donor sequences, and candidates with especially long matches between target sites and bridgeRNA target binding loops could be prioritized for experimental characterization.

### E. Donor and Target Sites

The donor and target site sequences may be any sequence that can be bound by the transposase in concert with bridgeRNA. For example, the bridgeRNA target binding loop encodes a LTG and RTG which have base-pairing specificity for sequences of the target site, i.e., the left target (LT) and right target (RT) sequences, in a target molecule. In some embodiments, the bridgeRNA donor binding loop encodes a LDG and RDG which have base-pairing specificity for sequences of the donor site, i.e., the left donor (LD) and right donor (RD) sequences, in a donor molecule, thereby positioning the transposase to mediate site-specific transposition between the DNA molecules comprising the target site and donor sites. The target site sequence and the donor site sequence may be on the same or different molecules. The binding loops of the bridgeRNA may also bind the core sequences of the target site or donor site sequences. As described above, certain mismatch and/or non-contiguous tolerance between the LTG and RTG and the target site sequences and/or the LDG and RDG and the donor site sequences may be acceptable.

Described herein is the use of IS110 family transposases to mediate insertion, excisive recombination, and inversion reactions. The type of reaction mediated by the IS110 family transposase is a function of the orientation and DNA strand location of the LT and RT target site sequences and LD and RD donor site sequences as described below.

Described herein is the use of wild-type bridgeRNAs or a bridgeRNA comprising a wild-type donor binding loop and/or target binding loop to mediate recombination between DNA sequences containing the wild-type target and donor site sequences. For example, where a donor site sequence for any of the IS110 family transposases described herein (see e.g., FIG. 17 (SEQ ID NOS: 30354-30529) or SEQ ID NOS: 30530-40356) is present on a DNA molecule and a corresponding target site sequence (see e.g., FIG. 18 (SEQ ID NOS: 20351-20526) or SEQ ID NOS: 20527-30353) is present on a DNA molecule, expression of said IS110 family transposase and corresponding bridgeRNA can result in insertion, excisive recombination, or inversion.

In some embodiments, wild-type bridgeRNAs or a bridgeRNA comprising a wild-type donor binding loop and target binding loop can be used to mediate an insertion, excisive recombination, or inversion reaction between DNA sequences containing the wild-type target site sequence and wild-type donor site sequence.

Also described herein, is reprogramming of bridgeRNA donor binding loop and/or target binding loop sequences to mediate transposition between DNA sequences of interest.

In some embodiments, a bridgeRNA comprising a wild-type donor binding loop can be used in combination with a reprogrammed target binding loop to mediate an insertion, excisive recombination, or inversion reaction between DNA sequences containing the wild-type donor site sequence and a target site sequence of interest. In some embodiments, a bridgeRNA comprising a reprogrammed donor binding loop can be used in combination with a wild-type target binding loop to mediate an insertion reaction between DNA sequences containing a donor site sequence of interest and the wild-type target site sequence.

In some embodiments, a bridgeRNA comprising a reprogrammed donor binding loop can be used in combination with a reprogrammed target binding loop to mediate an insertion reaction between DNA sequences containing a donor site sequence of interest and a target site sequence of interest.

In some embodiments, different bridgeRNAs (in combination with the same or different transposases) can be used to target more than one target site sequence or donor site sequence simultaneously.

### E.1. Insertion Reaction

To mediate insertion reactions, the target site and donor site sequences are present on separate DNA molecules. In the case that the donor site sequence is arranged on a circular DNA molecule, the transposition reaction functionally results in insertion of the circular DNA molecule that contains the donor site sequence into the target site sequence of the second DNA molecule. In the case that the target site sequence is arranged on a circular DNA molecule, the transposition reaction functionally results in insertion of the circular DNA molecule containing the target site sequence into the donor site sequence of the second DNA molecule. In the case that both the donor site sequence and target site sequence are on linear DNA molecules, the transposition reaction functionally results in insertion of the linear DNA molecule that contains the donor site sequence into the target site sequence of the second DNA molecule, the double stranded break that occurs after recombination with a linear DNA molecule is repaired by endogenous DNA repair pathways, such as non-homologous end joining. In the case that both the donor site sequence and target site sequence are on different linear chromosomes, the transposition reaction functionally results to effectuate chromosomal translocation without a double stranded break.

In embodiments where a core sequence is not present, to mediate insertion, the sequence of the LTG of the target binding loop in the 5′ to 3′ direction is complementary to a first strand of a target site sequence. The sequence of the RTG of the target binding loop in the 5′ to 3′ direction is the reverse complement to the opposite strand to the first strand of the target site sequence with the 3′ end of the RTG reverse complementary to the nucleotide immediately following the target site sequence complementary to the LTG. In some embodiments, the sequence of the RTG of the target binding loop in the 5′ to 3′ direction is the reverse complement to the opposite strand to the first strand of the target site sequence with the 3′ end of the RTG reverse complementary to the second or third nucleotide immediately following the target site sequence complementary to the LTG (i.e., there is 1-2 nucleotide gap between where LTG and RTG bind). The sequence of the LDG of the donor binding loop in the 5′ to 3′ direction is complementary to a first strand of the donor site sequence. The sequence of the RDG in the 5′ to 3′ direction is the reverse complement to the opposite strand to the first strand of the donor site sequence with the 3′ end of the RDG reverse complementary to the nucleotide immediately following the donor site sequence complementary to the LDG. In some embodiments, the sequence of the RDG in the 5′ to 3′ direction is the reverse complement to the opposite strand to the first strand of the donor site sequence with the 3′ end of the RDG reverse complementary to the second or third nucleotide immediately following the donor site sequence complementary to the LDG (i.e., there is 1-2 nucleotide gap between where LDG and RDG bind).

In some embodiments where a core sequence is present, to mediate insertion, the sequence of the LTG of the target binding loop in the 5′ to 3′ direction is complementary to a first strand of a target site sequence with the 3′ end of the LTG complementary to at least one of the nucleotides of the core sequence on the first strand of the target site sequence. The sequence of the RTG in the 5′ to 3′ direction is the reverse complement to the opposite strand to the first strand of the target site sequence with the 3′ end of the RTG reverse complementary to at least one of the nucleotides of the core sequence on the opposite strand to the first strand of the target site sequence. In some embodiments, there is 1-2 nucleotide gap between where LTG and RTG bind. The sequence of the LDG in the 5′ to 3′ direction is complementary to a first strand of the donor site sequence with the 3′ end of the LDG complementary to at least one of the nucleotides of the core sequence on the first strand of the donor site sequence. The sequence of the RDG in the 5′ to 3′ direction is the reverse complement to the opposite strand to the first strand of the donor site sequence with the 3′ end of the RDG reverse complementary to at least one of the nucleotides of the core sequence on the opposite strand to the first of the donor site sequence. In some embodiments, there is 1-2 nucleotide gap between where LDG and RDG bind. See FIG. 4, FIG. 35B.

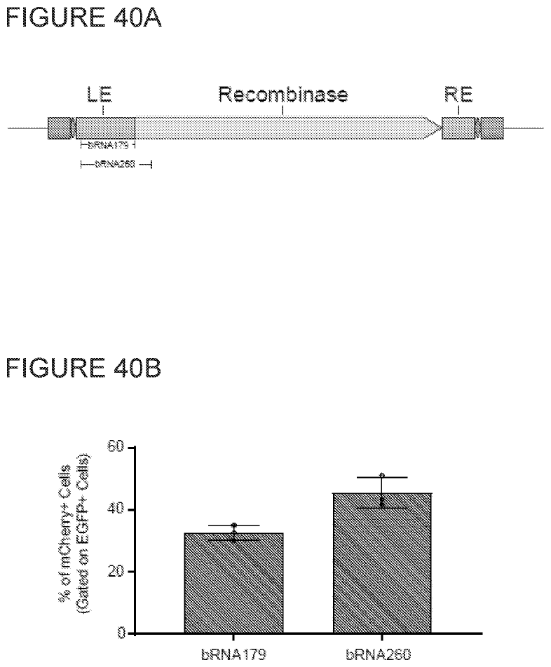

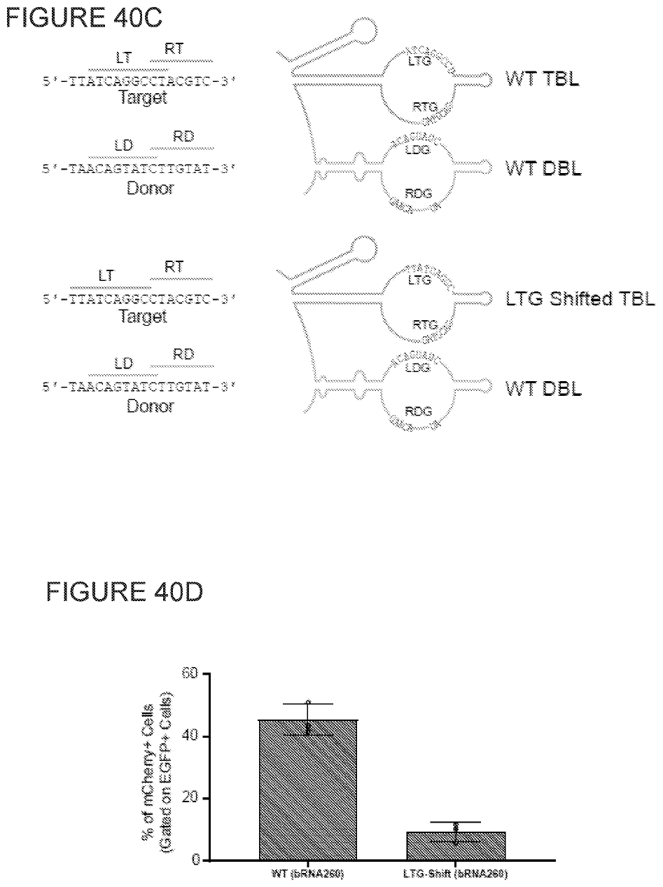

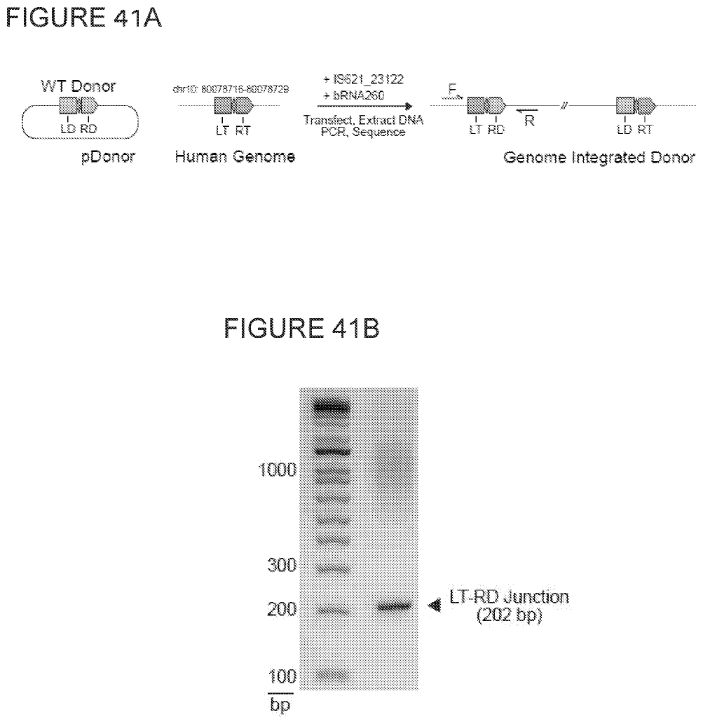

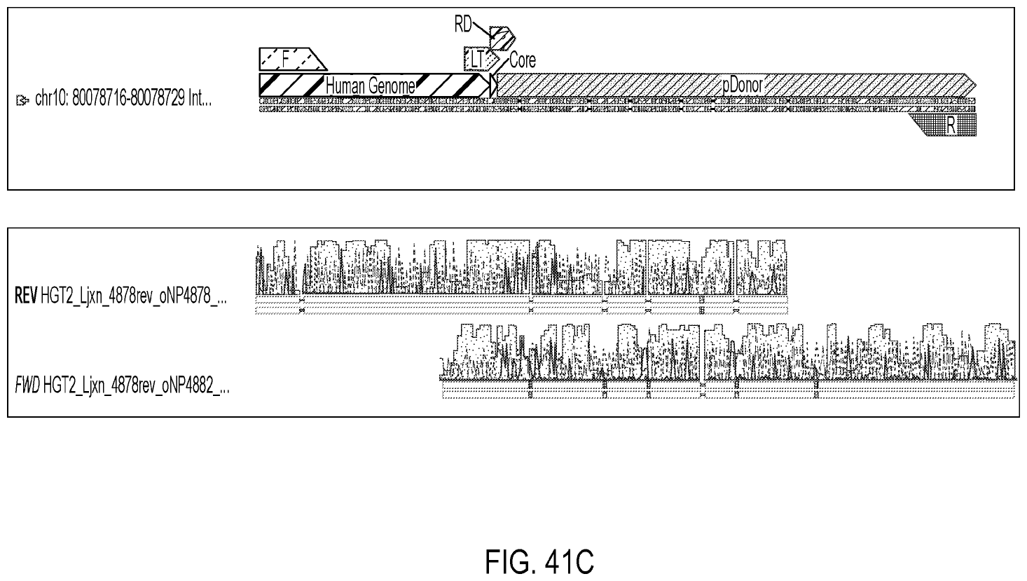

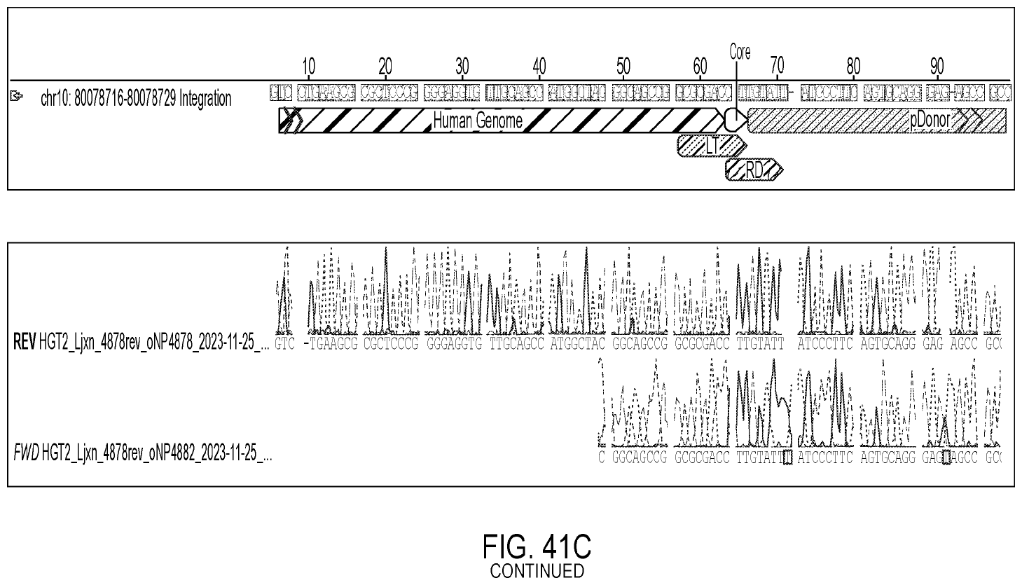

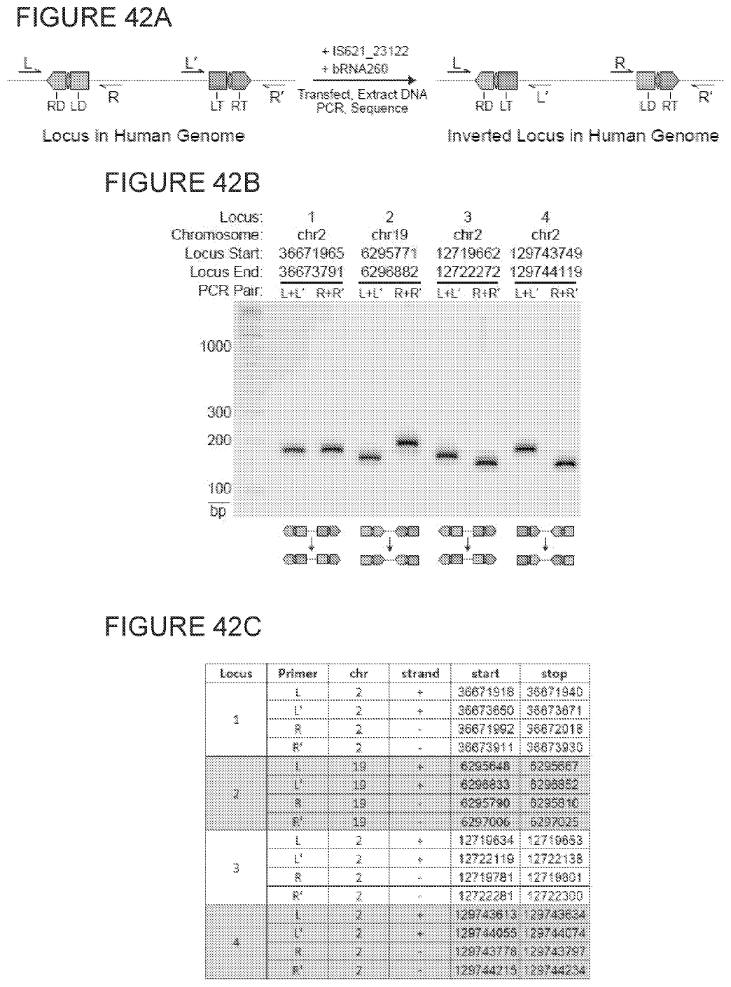

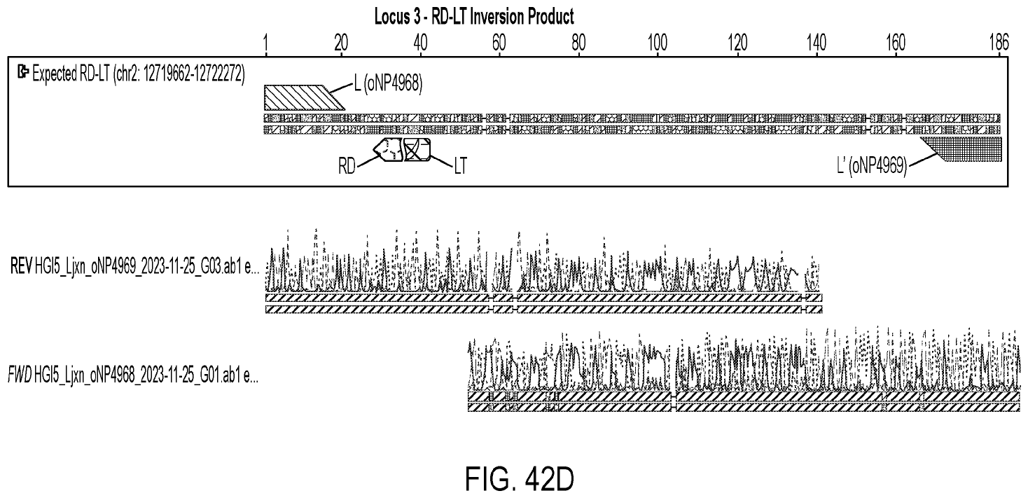

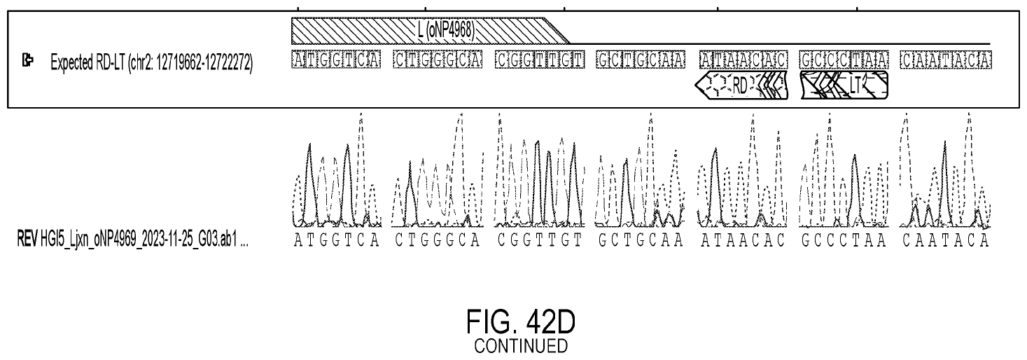

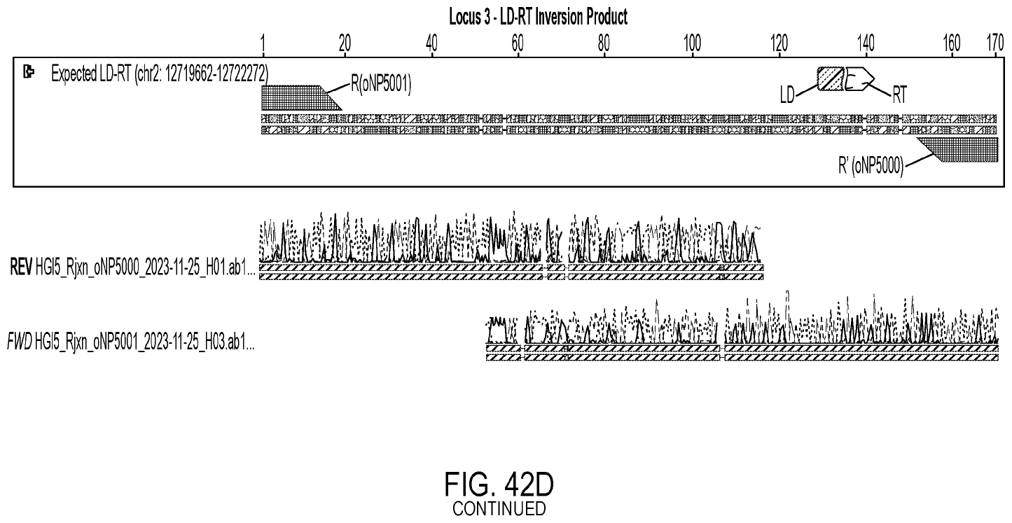

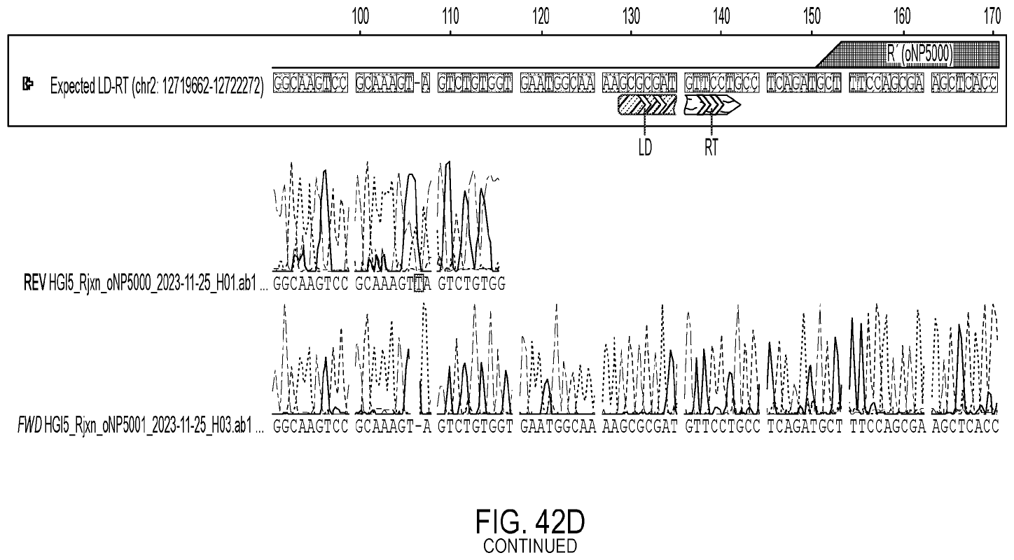

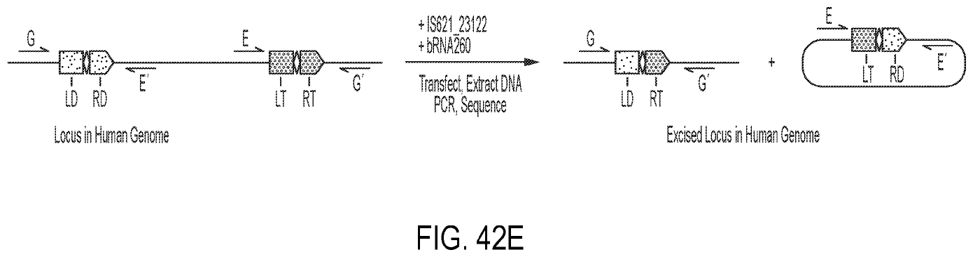

In some embodiments where a core sequence is present, to mediate insertion, the sequence of the LTG of the target binding loop in the 5′ to 3′ direction is complementary to a first strand of a target site sequence. The sequence of the RTG in the 5′ to 3′ direction is the reverse complement to the opposite strand to the first strand of the target site sequence. The sequence of the LDG in the 5′ to 3′ direction is complementary to the first strand of the donor site sequence. The sequence of the RDG in the 5′ to 3′ direction is the reverse complement to the opposite strand to the first strand of the donor site sequence. Thus, in this embodiment, the RTG, LTG, RDG, and LDG do not bind to the core sequence even though it is present. In some embodiments, one or more of RTG, LTG, RDG, and LDG can be complementary to at least one of the nucleotides of the core sequence. See e.g., FIG. 35B. Furthermore, in some embodiments, there is 1-2 nucleotide gap between where LDG and RDG bind and/or between where LTG and RTG bind. In some embodiments, a bridgeRNA has been engineered or modified so that one or more of the RTG, LTG, RDG, and LDG no longer bind to the core sequence even though it is present in the donor and target site sequences. For example, a naturally occurring bridgeRNA wherein one or more of the RTG, LTG, RDG, and LDG bind to the core sequence can be modified so that the one or more of the RTG, LTG, RDG, and LDG no longer bind to the core sequence even though it is remains present in the donor and target site sequences. Such an approach can increase the binding specificity of the bridgeRNA for the donor site sequence and/or target site sequence. See e.g., FIG. 40A-D.

In any of the above embodiments, the LTG and RTG sequences are fully complementary to their respective target site sequences of the target DNA. In some embodiments, the LTG and/or RTG are partially complementary to their respective target site sequences of the target DNA, i.e., there is non-canonical base pairing, mismatch and/or non-contiguous tolerance. In some embodiments, there is a single mismatch in the LTG or RTG nucleotide sequence. In some embodiments, there are two, three or four mismatches which can be in the LTG, in the RTG, or spread across the LTG and RTG. In some embodiments, the two, three or four mismatches are contiguous. In some embodiments, the two, three or four mismatches are non-contiguous. In some embodiments, there is a single non-canonical base pair in the LTG or RTG nucleotide sequence. In some embodiments, there are two, three or four non-canonical base pairs which can be in the LTG, in the RTG, or spread across the LTG and RTG. In some embodiments, the two, three or four non-canonical base pairs are contiguous. In some embodiments, the two, three or four non-canonical base pairs are non-contiguous. In some embodiments, the LDG and RDG nucleotide sequences are fully complementary to their respective donor site sequences of the donor DNA. In some embodiments, the LDG and/or RDG nucleotide sequences are partially complementary to their respective donor site sequences of the donor DNA, i.e., there is non-canonical base pairing, mismatch and/or non-contiguous tolerance. In some embodiments, there is a single mismatch in the LDG or RDG nucleotide sequence. In some embodiments, there are two, three, or four mismatches which can be in the LDG, in the RDG, or spread across the LDG and RDG. In some embodiments, the two, three, or four mismatches are contiguous. In some embodiments, the two, three, or four mismatches are non-contiguous. In some embodiments, there is a single non-canonical base pair in the LDG or RDG nucleotide sequence. In some embodiments, there are two, three, or four non-canonical base pairs which can be in the LDG, in the RDG, or spread across the LDG and RDG. In some embodiments, the two, three, or four non-canonical base pairs are contiguous. In some embodiments, the two, three, or four non-canonical base pairs are non-contiguous. In some embodiments, the non-canonical base pairing is non-Watson-Crick base pairing (i.e., is not a G-C base pair or A-T/U base pair). In some embodiments, the non-canonical base pairing is wobble base pairing. In some embodiments, the non-canonical base pairing is Hoogsteen base pairing. In some embodiments, the non-canonical base pairing comprises a rG-dT base pair, a rU-dG base pair, a rA-dC base pair, a rC-dA base pair, a rA-dG base pair, or a rG-dG base pair.

In some embodiments, it is not necessary to define the sequences of the donor site and/or target site or LT, RT, LD, and RD (and, therefore, LTG, RTG, LDG, and RDG). Instead an insertion reaction can be mediated between a DNA molecule comprising a donor site comprising wild-type RE-LE or a subsequence thereof, or, where core is used, RE-core-LE or a subsequence thereof of any of the IS110 family transposases described herein (see FIG. 15 (SEQ ID NOS: 1-348), FIG. 17 (SEQ ID NOS: 30354-30529), or SEQ ID NOS: 349-10175 or 30530-40356) and a DNA molecule comprising a target sequence comprising LF-RF or a subsequence thereof, or where core is used, LF-core-RF of any of the IS110 family transposases described herein (see FIG. 18 (SEQ ID NOS: 20351-20526), or SEQ ID NOS: 20527-30353) by providing the IS110 family transposase and its corresponding bridgeRNA. It is also not necessary to define the sequence of the bridgeRNA since bridgeRNA is encoded in LE or RE. Thus, in some embodiments, bridgeRNA sequence can be provided by providing the nucleotide sequence of LE or RE.

For insertion reactions described herein, the system may further comprise one or more polynucleotides for insertion (e.g., for insertion into a target site) comprising a cargo and a donor site sequence. In another embodiment, the system further comprises one or more polynucleotides for insertion (e.g., for insertion into a donor site) comprising a cargo sequence and a target site sequence. In some embodiments, the polynucleotide for insertion is circular. In some embodiments, the polynucleotide for insertion is linear. When a polynucleotide for insertion is linear, the double stranded break that occurs after recombination with a linear DNA molecule is repaired by endogenous DNA repair pathways, such as non-homologous end joining.

For insertion reactions that result in cassette exchange (e.g., recombinase mediated cassette exchange (RMCE)), in some embodiments, the one or more polynucleotides for insertion further comprises a second donor site sequence that is different from the first donor site sequence (i.e., the one or more polynucleotides for insertion comprises two donor site sequences). In some embodiments, the one or more polynucleotides for insertion further comprises a target site sequence that corresponds to a different donor site from the first donor site sequence (i.e., the one or more polynucleotides for insertion comprises a donor site sequence and a target site sequence). In some embodiments, the one or more polynucleotides for insertion further comprises a second target site sequence that is different from the first target site sequence (i.e., the one or more polynucleotides for insertion comprises two target site sequences). In some embodiments, the one or more polynucleotides for insertion further comprises a donor site sequence that corresponds to a different target site from the first target site sequence (i.e., the one or more polynucleotides for insertion comprises a donor site sequence and a target site sequence). In such embodiments, the system comprises a transposase with a first bridgeRNA that targets the first donor or target site sequence and a transposase with a second bridgeRNA that targets the second donor or target site sequence. In some embodiments, the transposase bound to the first bridgeRNA and second bridgeRNA are the same type, e.g. IS621. In some embodiments, the transposase bound to the first bridgeRNA and second bridgeRNA are different transposases.

In some embodiments, the system comprises components that effectuate at least two (and in some embodiments more than two) desired reactions. A polynucleotide for insertion comprising a cargo, a donor site sequence and a target site sequence, for example on a plasmid, is recombined using a bridgeRNA that targets the donor and target site sequences to create two mini circles, one with a LT-RD junction and the other with an LD-RT junction. A second bridgeRNA, that comprises a binding loop that has specificity for the newly formed LT-RD or LD-RT and a binding loop that targets a site in a genome of interest, binds the mini circle and integrates it into a target site sequence, such as into a genome of interest. Thus, for such insertion reactions, in some embodiments, the system may comprise one or more first circular polynucleotides (e.g. a plasmid) comprising a cargo, a first donor site sequence comprising LD and RD sequences, and a second target site sequence comprising LT and RT sequences. In some embodiments, the system further comprises a transposase with a first bridgeRNA that targets the first donor site sequence and the first target site sequence on the first polynucleotide and a second bridgeRNA that targets a second donor site sequence and a second target site sequence, wherein the second donor site sequence comprises LT of the first target site sequence and RD of the first donor site or LD of the first donor site and RT of the first target site and the second target site sequence is a site in a target of interest (e.g., a genome). The second bridgeRNA can target the second donor site sequence using a donor binding loop (i.e., a donor binding loop of the second bridgeRNA targets the second donor site sequence) or a target binding loop (i.e., a target binding loop of the second bridgeRNA targets the second donor site sequence) as long as the other loop targets the site of interest (e.g., a genome). In some embodiments, the system results in the recombination of the donor site sequence and target site sequence of the first polynucleotide to create a first minicircle comprising a LT-RD junction and a second minicircle with a LD-RT junction. Depending on the orientation of the cargo, the first donor site sequence and the first target site sequence, either the first minicircle comprises the cargo or the second minicircle comprises the cargo and the second bridgeRNA targets whichever minicircle comprises the cargo. In some embodiments, the system results in the recombination of the either the first or second minicircle, whichever comprises the cargo, with the site of interest to result in the cargo inserted at the site of interest.

In some embodiments, the first circular polynucleotide further comprises a second cargo and the system further comprises a third bridgeRNA that targets a third donor site sequence and a third target site sequence, wherein the third donor site sequence comprises LT of the first target site sequence and RD of the first donor site or LD of the first donor site and RT of the first target site (e.g., whichever of LT-RD or LD-RT not targeted by the second bridgeRNA) and the second target site sequence is a second site in a target of interest (e.g., a genome). The third bridgeRNA can target the third donor site sequence using a donor binding loop (i.e., a donor binding loop of the third bridgeRNA targets the third donor site sequence) or a target binding loop (i.e., a target binding loop of the third bridgeRNA targets the third donor site sequence) as long as the other loop targets the second site of interest (e.g., a genome). In some embodiments, the system results in the recombination of the donor site sequence and target site sequence of the first polynucleotide to create a first minicircle comprising a LT-RD junction and either the first cargo and a second minicircle with a LD-RT junction and either the second cargo, or vice versa (e.g., second cargo comprised on the minicircle with LT-RD junction and first cargo comprised on the minicircle with LD-RT junction). In some embodiments, the system results in the recombination of the first minicircle with the first site in a target of interest and recombination of the second minicircle with the second site in a target of interest, or vice versa, with the site of interest to result in the first cargo and second cargos inserted at the respective sites of interest.

In some embodiments, in the system comprising one or more polynucleotides for insertion comprising a cargo and a donor site sequence, the cargo sequence is oriented in the 5′ to 3′ direction relative to the donor site sequence. In some embodiments, in the system comprising one or more polynucleotides for insertion comprising a cargo and a donor site, the cargo sequence is oriented in the 3′ to 5′ direction relative to the donor site sequence. In some embodiments, in the system comprising one or more polynucleotides for insertion comprising a cargo and two donor site sequences, the cargo sequence is oriented in the 5′ to 3′ direction between the two donor site sequences. In some embodiments, in the system comprising one or more polynucleotides for insertion comprising a cargo and two donor site sequences, the cargo sequence is oriented in the 3′ to 5′ direction between the two donor site sequences. In some embodiments, in the system comprising one or more polynucleotides for insertion comprising a cargo and a donor site sequence and a target site sequence that corresponds to a different donor site, the cargo sequence is oriented in the 5′ to 3′ direction between the donor site sequence and target site sequence. In some embodiments, in the system comprising one or more polynucleotides for insertion comprising a cargo and a donor site sequence and a target site sequence that corresponds to a different donor site, the cargo sequence is oriented in the 3′ to 5′ direction between the donor site sequence and target site sequence.

In some embodiments, in the system comprising one or more polynucleotides for insertion comprising a cargo and a target site sequence, the cargo sequence is oriented in the 5′ to 3′ direction relative to the target site sequence. In some embodiments, in the system comprising one or more polynucleotides for insertion comprising a cargo and a target site sequence, the cargo sequence is oriented in the 3′ to 5′ direction relative to the target site sequence. In some embodiments, in the system comprising one or more polynucleotides for insertion comprising a cargo and two target site sequences, the cargo sequence is oriented in the 5′ to 3′ direction between the two target site sequences. In some embodiments, in the system comprising one or more polynucleotides for insertion comprising a cargo and two target site sequences, the cargo sequence is oriented in the 3′ to 5′ direction between the two target site sequences. In some embodiments, in the system comprising one or more polynucleotides for insertion comprising a cargo and a target site sequence and a donor site sequence that corresponds to a different target site, the cargo sequence is oriented in the 5′ to 3′ direction between the target site sequence and donor site sequence. In some embodiments, in the system comprising one or more polynucleotides for insertion comprising a cargo and target site sequences and donor site sequence, the cargo sequence is oriented in the 3′ to 5′ direction between the target site sequences and donor site sequence.

A polynucleotide for insertion may be an equivalent of a transposable element that can be inserted or integrated to a target site sequence or donor site sequence. The polynucleotide for insertion may be or comprise one or more components of a transposon.

The cargo of a polynucleotide for insertion may comprise any type of polynucleotide, including, but not limited to, a gene, a gene fragment, a non-coding polynucleotide, a regulatory polynucleotide, or a synthetic polynucleotide.

The polynucleotide for insertion may include a transposon left end (LE) and transposon right end (RE). The LE or RE sequences may be endogenous sequences for the IS110 used or may be heterologous sequences recognizable by the IS110 used, or may be synthetic sequences that comprise a sequence or structure feature recognized by the IS110 and sufficient to allow insertion of the polynucleotide into the target site sequence. In certain example embodiments, the LE or RE sequences are truncated. In certain example embodiments, the LE or RE sequences may be between 20-500 base pairs, between 500-490 base pairs, between 500-480 base pairs, between 500-470 base pairs, between 500-460 base pairs, between 500-450 base pairs, between 500-440 base pairs, between 500-430 base pairs, between 500-420 base pairs, between 500-410 base pairs, between 500-400 base pairs, between 400-390 base pairs, between 400-380 base pairs, between 400-370 base pairs, between 400-360 base pairs, between 400-350 base pairs, between 400-340 base pairs, between 400-330 base pairs, between 400-320 base pairs, between 400-310 base pairs, between 400-300 base pairs, between 300-290 base pairs, between 300-280 base pairs, between 300-270 base pairs, between 300-260 base pairs, between 300-250 base pairs, between 300-240 base pairs, between 300-230 base pairs, between 300-220 base pairs, between 300-210 base pairs, between 300-200 base pairs, between 200-100 base pairs, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length. In some embodiments, the polynucleotide for insertion may include a transposon LD and transposon RD.

The polynucleotide for insertion may include a transposon left flank (LF) and transposon right flank (RF). The LF or RF sequences may be endogenous sequences for the IS110 used or may be heterologous sequences recognizable by the IS110 used, or may be synthetic sequences that comprise a sequence or structure feature recognized by the IS110 and sufficient to allow insertion of the polynucleotide into the donor site. In certain example embodiments, the LF or RF sequences are truncated. In certain example embodiments, the LF or RF sequences may be between 20-500 base pairs, between 500-490 base pairs, between 500-480 base pairs, between 500-470 base pairs, between 500-460 base pairs, between 500-450 base pairs, between 500-440 base pairs, between 500-430 base pairs, between 500-420 base pairs, between 500-410 base pairs, between 500-400 base pairs, between 400-390 base pairs, between 400-380 base pairs, between 400-370 base pairs, between 400-360 base pairs, between 400-350 base pairs, between 400-340 base pairs, between 400-330 base pairs, between 400-320 base pairs, between 400-310 base pairs, between 400-300 base pairs, between 300-290 base pairs, between 300-280 base pairs, between 300-270 base pairs, between 300-260 base pairs, between 300-250 base pairs, between 300-240 base pairs, between 300-230 base pairs, between 300-220 base pairs, between 300-210 base pairs, between 300-200 base pairs, between 200-100 base pairs, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length. In some embodiment, the polynucleotide for insertion may include a transposon LT and transposon RT.

The term “cargo”, “cargo gene” or “cargo sequence” as used herein, refers to a gene or nucleic acid sequence that may be integrated via transposition into a target site sequence or donor site sequence. The term “cargo(es) to be delivered”, “cargo gene(s) to be delivered” or “cargo sequence(s) to be delivered” refers to any gene, system of genes, regulatory sequences, or sequences that can be delivered to and integrated into a target site sequence or donor site sequence via transposition events. In some embodiments, the cargo gene or sequence is to be delivered to a target cell in vitro, in vivo, or ex vivo.

In some embodiments, the cargo gene or sequence to be delivered is a biologically active agent, i.e., it has activity in a cell, organ, tissue, and/or subject. For instance, a gene or sequence that, when administered to a subject, has a biological effect on that subject, is considered to be biologically active. In some embodiments, a cargo gene or sequence to be delivered is a therapeutic agent. As used herein, the term “therapeutic agent” refers to any agent that, when administered to a subject, has a beneficial effect. In some embodiments, the cargo gene or sequence to be delivered to a cell is a transcription factor, a tumor suppressor, a developmental regulator, a growth factor, a metastasis suppressor, a pro-apoptotic protein, a nuclease, or a recombinase. In some embodiments, the cargo gene or sequence to be encodes for a protein, some non-limiting examples include, p53, Rb (retinoblastoma protein), BRCA1, BRCA2, PTEN, APC, CD95, ST7, ST14, a BCL-2 family protein, a caspase; BRMS1, CRSP3, DRG1, KAI1, KISS1, NM23, a TIMP-family protein, a BMP-family growth factor, EGF, EPO, FGF, G-CSF, GM-CSF, a GDF-family growth factor, HGF, HDGF, IGF, PDGF, TPO, TGF-α, TGF-β, VEGF; a zinc finger nuclease, Cre, Dre, or FLP recombinase. In some embodiments, the cargo gene or sequence is associated with a small molecule. In some embodiments, the cargo gene or sequence to be delivered is a diagnostic agent. In some embodiments, the cargo gene or sequence to be delivered is a prophylactic agent. In some embodiments, the cargo gene or sequence to be delivered is useful as an imaging agent. In some embodiments, the diagnostic or imaging agent is, and in others it is not, biologically active.

In some embodiments, the polynucleotide for insertion comprises from 11 bases (b) or base pairs (bp) to about 100 kilobases (kb) or kilobase pairs (kbp) in length or higher (e.g., from about 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, or 100 b or bp to about 110, 120, 125, 150, 200, 225, 250, 275, 300, 325, 350, 375, 400, 425, 450, 475, 500, 525, 550, 575, 600, 650, 700, 750, 800, 850, 900, 950, 1000, 1250, 1500, 1750, 2000, 2250, 2500, 2750, 3000, 3250, 3500, 3750, 4000, 4250, 4500, 4750, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 16,000, 17,000, 18,000, 19,000, 20,000, 21,000, 22,000, 23,000, 24,000, 25,000, 26,000, 27,000, 28,000, 29,000 30,000, 40,000, 50,000, 60,000, 70,000, 80,000, 90,000, or 100,000 b or bp in length) with the upper limit corresponding to the delivery limit for delivering the polynucleotide for insertion into a cell of interest.

Polynucleotide for insertion can be delivered as dsDNA, or as ssDNA or RNA if cellular machinery, or additional components are delivered, to make these molecules into dsDNA. Polynucleotides can be provided in the form of a circular or linearized plasmid or as a component of a vector (e.g., as a component of a viral vector), or an amplification or polymerization product thereof. Shorter DNA molecules can be provided as double stranded oligonucleotides. Exemplary double-stranded template oligonucleotides are, or are least about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 115, 120, 125, 150, 175, 200, 225, or 250 b or bp in length. The polynucleotide for insertion can be provided in the reaction mixture for introduction into the cell at a concentration of from about 1 μM to about 200 μM, from about 2 μM to about 190 μM, from about 2 μM to about 180 μM, from about 5 μM to about 180 μM, from about 9 μM to about 180 μM, from about 10 μM to about 150 μM, from about 20 μM to about 140 μM, from about 30 μM to about 130 μM, from about 40 μM to about 120 μM, or from about 45 or 50 μM to about 90 or 100 μM. In some cases, the donor DNA can be provided in the reaction mixture for introduction into the cell at a concentration of, or of about, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 50 μM, 55 μM, 60 μM, 70 μM, 80 μM, 90 μM, 100 μM, 110 μM, 115 μM, 120 μM, 130 μM, 140 μM, 150 μM, 160 μM, 170 μM, 180 μM, 190 μM, 200 μM, or more.

The polynucleotide for insertion can contain a wide variety of different sequences. In some cases, the polynucleotide encodes a stop codon, or frame shift, as compared to the target genomic region prior to insertion. Such a polynucleotide can be useful for knocking out or inactivating a gene or portion thereof. In some cases, the polynucleotide encodes one or more missense mutations or in-frame insertions or deletions as compared to the target genomic region. Such a polynucleotide can be useful for altering the expression level or activity (e.g., ligand specificity) of a target gene or portion thereof.

As described above, a polynucleotide for insertion comprises a donor site sequence and/or target site sequence for insertion into a DNA sequence comprising a target site sequence and/or donor site sequence, respectively. The target site sequence and/or donor site sequence for insertion into can be located on any polynucleotide sequence of interest, including, but not limited to genomic DNA and plasmids. In some embodiments, the target site sequence and/or donor site sequence for insertion into is a polynucleotide sequence present in the genome or DNA of interest. In some embodiments the target site sequence and/or donor site sequence naturally occurs in the genome or DNA of interest. In some embodiments, the target site sequence and/or donor site sequence for insertion into is introduced into the genome or a DNA of interest. Methods of introducing DNA sequences, such as the target site sequence or donor site sequence, into a genome or DNA of interest are known in the art and include, but are not limited to, CRISPR-Cas9, homology directed repair (HDR), transposases, integrases, etc. In some embodiments, the genomic DNA is located in a cell. In some embodiments, the cell is a eukaryotic cell. In some embodiments, the cell is prokaryotic. In some embodiments, the cell is a mammalian cell. In some embodiments, the cell is a human cell. In some embodiments, the cell is a mouse cell. In some embodiments, the cell is a stem cell.

The target site sequence for insertion into may include a transposon left flank (LF) and transposon right flank (RF). The LF or RF sequences may be endogenous sequences for the IS110 used or may be heterologous sequences recognizable by the IS110 used, or may be synthetic sequences that comprise a sequence or structure feature recognized by the IS110 and sufficient to allow insertion of the polynucleotide into the donor site sequence. In certain example embodiments, the LF or RF sequences are truncated. In certain example embodiments, the LF or RF sequences may be between 20-500 base pairs, between 500-490 base pairs, between 500-480 base pairs, between 500-470 base pairs, between 500-460 base pairs, between 500-450 base pairs, between 500-440 base pairs, between 500-430 base pairs, between 500-420 base pairs, between 500-410 base pairs, between 500-400 base pairs, between 400-390 base pairs, between 400-380 base pairs, between 400-370 base pairs, between 400-360 base pairs, between 400-350 base pairs, between 400-340 base pairs, between 400-330 base pairs, between 400-320 base pairs, between 400-310 base pairs, between 400-300 base pairs, between 300-290 base pairs, between 300-280 base pairs, between 300-270 base pairs, between 300-260 base pairs, between 300-250 base pairs, between 300-240 base pairs, between 300-230 base pairs, between 300-220 base pairs, between 300-210 base pairs, between 300-200 base pairs, between 200-100 base pairs, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length. In some embodiments, the target site sequence for insertion into may include a transposon left target (LT) and transposon right target (RT).

The donor site sequence for insertion into may include a transposon left end (LE) and transposon right end (RE). The LE or RE sequences may be endogenous sequences for the IS110 used or may be heterologous sequences recognizable by the IS110 used, or may be synthetic sequences that comprise a sequence or structure feature recognized by the IS110 and sufficient to allow insertion of the polynucleotide into the donor site sequence. In certain example embodiments, the LE or RE sequences are truncated. In certain example embodiments, the LE or RE sequences may be between 20-500 base pairs, between 500-490 base pairs, between 500-480 base pairs, between 500-470 base pairs, between 500-460 base pairs, between 500-450 base pairs, between 500-440 base pairs, between 500-430 base pairs, between 500-420 base pairs, between 500-410 base pairs, between 500-400 base pairs, between 400-390 base pairs, between 400-380 base pairs, between 400-370 base pairs, between 400-360 base pairs, between 400-350 base pairs, between 400-340 base pairs, between 400-330 base pairs, between 400-320 base pairs, between 400-310 base pairs, between 400-300 base pairs, between 300-290 base pairs, between 300-280 base pairs, between 300-270 base pairs, between 300-260 base pairs, between 300-250 base pairs, between 300-240 base pairs, between 300-230 base pairs, between 300-220 base pairs, between 300-210 base pairs, between 300-200 base pairs, between 200-100 base pairs, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length. The donor site sequence for insertion into may include a transposon LD and transposon RD.

### E.2. Excisive Recombination and Inversion

To mediate excisive recombination or inversion reactions, the target site sequence and donor site sequence are present on a single DNA molecule. When the target site sequence and donor site sequence are present on a single DNA molecule, and in the case that the target site sequence and donor site sequence are arranged such that the LT and LD are on the same DNA strand and the RT and RD are on the same DNA strand, the insertion reaction functionally results in excision of the DNA sequence (excisive recombination) intervening the target and donor site. When the target site sequence and donor site sequence are present on a single DNA molecule, and in the case that the target site sequence and donor site sequence are arranged such that the LT and RD are on the same DNA strand and the RT and LD are on the same DNA strand the insertion reaction functionally results in inversion of the DNA sequence intervening the target and donor site. In some embodiments where a core sequence is present, the core sequences of the donor site sequence and target site sequence are on opposite strands. Inversion reactions on a chromosomal scale can be referred to as intrachromosomal translocations.

In some embodiments, where a core sequence is not present, to mediate excisive recombination, the sequence of the LTG of the target binding loop in the 5′ to 3′ direction is complementary to a first strand of a target site sequence. The sequence of the RTG of the target binding loop in the 5′ to 3′ direction is the reverse complement to the opposite strand to the first strand of the target site sequence, with the 3′ end of the RTG reverse complementary to the nucleotide immediately following the target site sequence complementary to the LTG. In some embodiments, the sequence of the RTG of the target binding loop in the 5′ to 3′ direction is the reverse complement to the opposite strand to the first strand of the target site sequence, with the 3′ end of the RTG reverse complementary to the second or third nucleotide immediately following the target site sequence complementary to the LTG (i.e., there is 1-2 nucleotide gap between where LTG and RTG bind). The sequence of the LDG of the donor binding loop in the 5′ to 3′ direction is complementary to a first strand of the donor site sequence. The sequence of the RDG in the 5′ to 3′ direction is the reverse complement to the opposite strand to the first strand of the donor site sequence with the 3′ end of the RDG reverse complementary to the nucleotide immediately following the target site sequence complementary to the LDG. In some embodiments, the sequence of the RDG in the 5′ to 3′ direction is the reverse complement to the opposite strand to the first strand of the donor site sequence with the 3′ end of the RDG reverse complementary to the second or third nucleotide immediately following the target site sequence complementary to the LDG (i.e., there is 1-2 nucleotide gap between where LDG and RDG bind). The first strand of the target site sequence and donor site sequence are on the same strand of the same DNA molecule. The donor and target sites can be any distance apart on a DNA molecule to result in excision of shorter DNA fragments (e.g. from about 11 bases or base pairs) up to many kilobases of DNA (e.g. the length of a chromosome) (e.g., from about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, or 100 b or bp to about 110, 120, 125, 150, 200, 225, 250, 275, 300, 325, 350, 375, 400, 425, 450, 475, 500, 525, 550, 575, 600, 650, 700, 750, 800, 850, 900, 950, 1000, 1250, 1500, 1750, 2000, 2250, 2500, 2750, 3000, 3250, 3500, 3750, 4000, 4250, 4500, 4750, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 16,000, 17,000, 18,000, 19,000, 20,000, 21,000, 22,000, 23,000, 24,000, 25,000, 26,000, 27,000, 28,000, 29,000, 30,000 b or bp in length, or up to the length of a chromosome in a cell of interest).

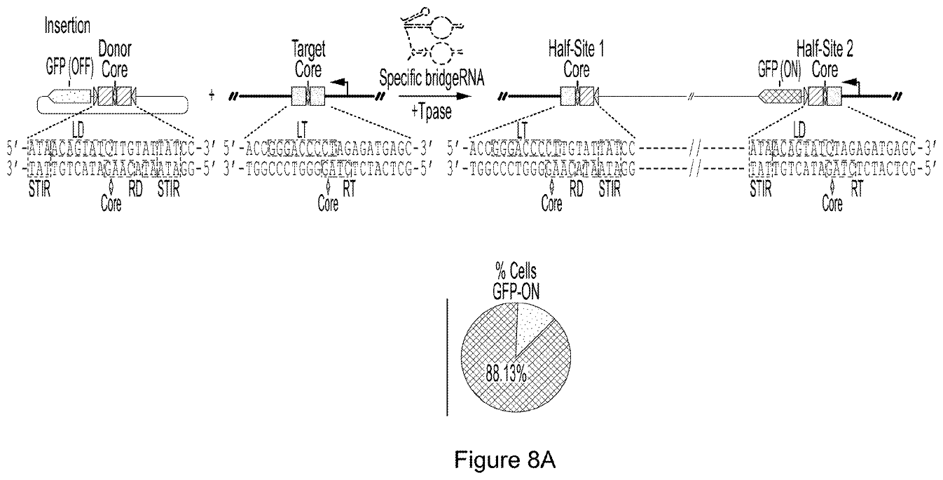

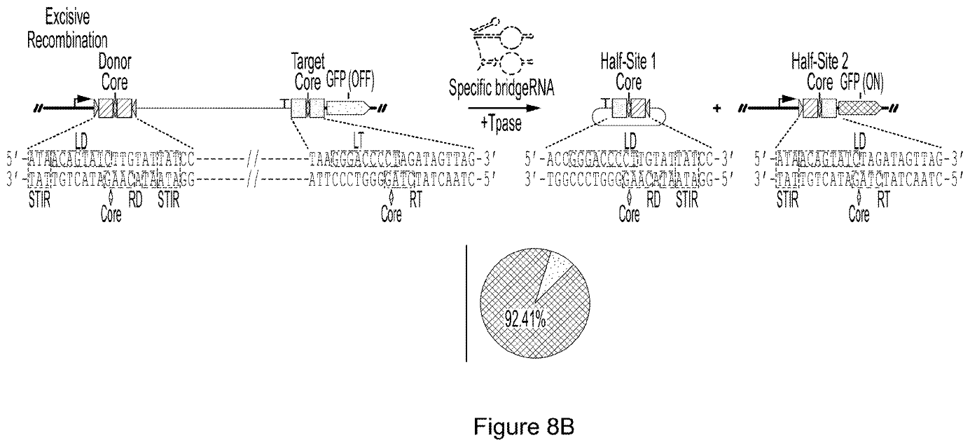

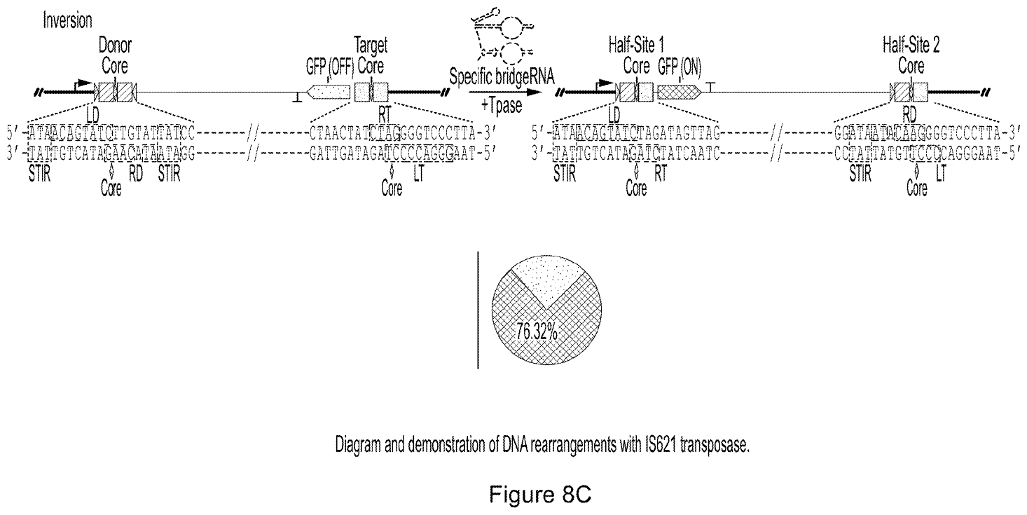

In some embodiments where a core sequence is present, to mediate excisive recombination, the sequence of the LTG of the target binding loop in the 5′ to 3′ direction is complementary to a first strand of a target site sequence with the 3′ end of the LTG complementary to at least one of the nucleotides of the core sequence on the first strand of the target site sequence. The sequence of the RTG in the 5′ to 3′ direction is the reverse complement to the opposite strand to the first strand of the target site sequence with the 3′ end of the RTG reverse complementary to at least one of the nucleotides of the core sequence on the opposite strand to the first strand of the target site sequence. In some embodiments, there is 1-2 nucleotide gap between where LTG and RTG bind. The sequence of the LDG in the 5′ to 3′ direction is complementary to a first strand of the donor site sequence with the 3′ end of the LDG complementary to at least one of the nucleotides of the core sequence on the opposite strand to the first strand of the donor site sequence. The sequence of the RDG in the 5′ to 3′ direction is the reverse complement to the opposite strand to the first strand of the donor site sequence with the 3′ end of the RDG reverse complementary to at least one of the nucleotides of the core sequence on the opposite strand to the first strand of the donor site sequence. See FIG. 8B. In some embodiments, there is 1-2 nucleotide gap between where LDG and RDG bind.

In some embodiments where a core sequence is present, to mediate excisive recombination, the sequence of the LTG of the target binding loop in the 5′ to 3′ direction is complementary to a first strand of a target site sequence. The sequence of the RTG in the 5′ to 3′ direction is the reverse complement to the opposite strand to the first strand of the target site sequence. The sequence of the LDG in the 5′ to 3′ direction is complementary to a first strand of the donor site sequence. The sequence of the RDG in the 5′ to 3′ direction is the reverse complement to the opposite strand to the first strand of the donor site sequence. Thus, in this embodiment, the RTG, LTG, RDG, and LDG do not bind to the core sequence even though it is present. In some embodiments, one or more of RTG, LTG, RDG, and LDG can be complementary to at least one of the nucleotides of the core sequence. See e.g., FIG. 35B. Furthermore, in some embodiments, there is 1-2 nucleotide gap between where LDG and RDG bind and/or between where LTG and RTG bind.

In any of these embodiments, the first strand of the target site sequence and donor site sequence are on the same strand of the same DNA molecule. The donor and target site sequences can be any distance apart on a DNA molecule to result in excision of shorter DNA fragments (e.g. from about 11 bases or base pairs) up to many kilobases of DNA (e.g. the length of a chromosome) (e.g., from about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, or 100 b or bp to about 110, 120, 125, 150, 200, 225, 250, 275, 300, 325, 350, 375, 400, 425, 450, 475, 500, 525, 550, 575, 600, 650, 700, 750, 800, 850, 900, 950, 1000, 1250, 1500, 1750, 2000, 2250, 2500, 2750, 3000, 3250, 3500, 3750, 4000, 4250, 4500, 4750, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 16,000, 17,000, 18,000, 19,000, 20,000, 21,000, 22,000, 23,000, 24,000, 25,000, 26,000, 27,000, 28,000, 29,000, 30,000 b or bp in length, or up to the length of a chromosome in a cell of interest).

In some embodiments, where a core sequence is not present, to mediate inversion, the sequence of the LTG of the target binding loop in the 5′ to 3′ direction is reverse complementary to the opposite strand to the first strand of a target site sequence. The sequence of the RTG of the target binding loop in the 5′ to 3′ direction is complementary to the first strand of the target site sequence with the 3′ end of the RTG reverse complementary to the nucleotide immediately following the target site sequence complementary to the LTG. In some embodiments, the sequence of the RTG of the target binding loop in the 5′ to 3′ direction is complementary to the first strand of the target site sequence with the 3′ end of the RTG reverse complementary to the second or third nucleotide immediately following the target site sequence complementary to the LTG (i.e., there is 1-2 nucleotide gap between where LTG and RTG bind). The sequence of the LDG of the donor binding loop in the 5′ to 3′ direction is complementary to the first strand of the donor site sequence. The sequence of the RDG in the 5′ to 3′ direction is the reverse complement to the opposite strand to the first strand of the donor site sequence with the 3′ end of the RDG reverse complementary to the nucleotide immediately following the target site sequence complementary to the LDG. In some embodiments, the sequence of the RDG in the 5′ to 3′ direction is the reverse complement to the opposite strand to the first strand of the donor site sequence with the 3′ end of the RDG reverse complementary to the second or third nucleotide immediately following the target site sequence complementary to the LDG (i.e., there is 1-2 nucleotide gap between where LDG and RDG bind). The first strand of the target site and donor site sequence are on the same strand of the same DNA molecule. The donor and target sites can be any distance apart on a DNA molecule to result in inversion of shorter DNA fragments (e.g. from about 11 bases or base pairs) up to many kilobases of DNA (e.g. the length of a chromosome) (e.g., from about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, or 100 b or bp to about 110, 120, 125, 150, 200, 225, 250, 275, 300, 325, 350, 375, 400, 425, 450, 475, 500, 525, 550, 575, 600, 650, 700, 750, 800, 850, 900, 950, 1000, 1250, 1500, 1750, 2000, 2250, 2500, 2750, 3000, 3250, 3500, 3750, 4000, 4250, 4500, 4750, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 16,000, 17,000, 18,000, 19,000, 20,000, 21,000, 22,000, 23,000, 24,000, 25,000, 26,000, 27,000, 28,000, 29,000, 30,000 b or bp in length, or up to the length of a chromosome in a cell of interest).

In some embodiments where a core sequence is present, to mediate inversion, the sequence of the LTG of the target binding loop in the 5′ to 3′ direction is reverse complementary to the opposite strand to the first strand of a target site sequence with the 3′ end of the LTG reverse complementary to at least one of the nucleotides of the core sequence on the opposite strand to the first strand of the target site sequence. The sequence of the RTG in the 5′ to 3′ direction is complementary to a first strand of the target site sequence with the 3′ end of the RTG complementary to at least one of the nucleotides of the core sequence on the first strand of the target site sequence. In some embodiments, there is 1-2 nucleotide gap between where LTG and RTG bind. The sequence of the LDG in the 5′ to 3′ direction is complementary to a first strand of the donor site sequence with the 3′ end of the LDG complementary to at least one of the nucleotides of the core sequence on the first strand of the donor site sequence. The sequence of the RDG in the 5′ to 3′ direction is the reverse complement to the opposite strand to the first strand of the donor site sequence with the 3′ end of the RDG reverse complementary to at least one of the nucleotides of the core sequence on the opposite strand to the first strand of the donor site sequence. See FIG. 8C. In some embodiments, there is 1-2 nucleotide gap between where LDG and RDG bind.

In some embodiments where a core sequence is present, to mediate inversion, the sequence of the LTG of the target binding loop in the 5′ to 3′ direction is reverse complementary to the opposite strand to the first strand of a target site sequence. The sequence of the RTG in the 5′ to 3′ direction is complementary to a first strand of the target site sequence. The sequence of the LDG in the 5′ to 3′ direction is complementary to a first strand of the donor site sequence. The sequence of the RDG in the 5′ to 3′ direction is the reverse complement to the opposite strand to the first strand of the donor site sequence. Thus, in this embodiment, the RTG, LTG, RDG, and LDG do not bind to the core sequence even though it is present. In some embodiments, one or more of RTG, LTG, RDG, and LDG can be complementary to at least one of the nucleotides of the core sequence. See e.g., FIG. 35B. Furthermore, in some embodiments, there is 1-2 nucleotide gap between where LDG and RDG bind and/or between where LTG and RTG bind.

In any of these embodiments, the first strand of the target site and donor site sequences are on the same strand of the same DNA molecule. The donor and target sites can be any distance apart on a DNA molecule to result in inversion of shorter DNA fragments (e.g. from about 11 bases or base pairs) up to many kilobases of DNA (e.g. the length of a chromosome) (e.g., from about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, or 100 b or bp to about 110, 120, 125, 150, 200, 225, 250, 275, 300, 325, 350, 375, 400, 425, 450, 475, 500, 525, 550, 575, 600, 650, 700, 750, 800, 850, 900, 950, 1000, 1250, 1500, 1750, 2000, 2250, 2500, 2750, 3000, 3250, 3500, 3750, 4000, 4250, 4500, 4750, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 16,000, 17,000, 18,000, 19,000, 20,000, 21,000, 22,000, 23,000, 24,000, 25,000, 26,000, 27,000, 28,000, 29,000, 30,000 b or bp in length, or up to the length of a chromosome in a cell of interest).

In any of the above embodiments, the LTG and RTG sequences are fully complementary to their respective target site sequences of the target DNA. In some embodiments, the LTG and/or RTG are partially complementary to their respective target site sequences of the target DNA, i.e., there is mismatch and/or non-contiguous tolerance. In some embodiments, there is a single mismatch in the LTG or RTG nucleotide sequence. In some embodiments, there are two, three or four mismatches which can be in the LTG, in the RTG, or spread across the LTG and RTG. In some embodiments, the two, three or four mismatches are contiguous. In some embodiments, the two, three or four mismatches are non-contiguous. In some embodiments, the LDG and RDG nucleotide sequences are fully complementary to their respective donor site sequences of the donor DNA. In some embodiments, the LDG and/or RDG nucleotide sequences are partially complementary to their respective donor site sequences of the donor DNA, i.e., there is mismatch and/or non-contiguous tolerance. In some embodiments, there is a single mismatch in the first or second nucleotide sequence. In some embodiments, there are two mismatches which can be in the LDG, in the RDG, or spread across the LDG and RDG. In some embodiments, the two mismatches are contiguous. In some embodiments, the two mismatches are non-contiguous.

In some embodiments, it is not necessary to define the sequences of LT, RT, LD, and RD (and, therefore, LTG, RTG, LDG, and RDG). Instead an excisive recombination or inversion reaction can be mediated between a DNA molecule comprising a donor site sequence comprising wild-type RE-LE or a subsequence thereof, or, where core is used, RE-core-LE or a subsequence thereof of any of the IS110 family transposases described herein (see FIG. 15 (SEQ ID NOS: 1-348), FIG. 17 (SEQ ID NOS: 30354-30529), or SEQ ID NOS: 349-10175 or 30530-40356), a DNA molecule comprising a target site sequence comprising LF-RF or a subsequence thereof, or where core is used, LF-core-RF of any of the IS110 family transposases described herein (see FIG. 18 (SEQ ID NOS: 20351-20526), or SEQ ID NOS: 20527-30353) by providing the IS110 family transposase and its corresponding bridgeRNA. It is also not necessary to define the sequence of the bridgeRNA since bridgeRNA is encoded in LE or RE. Thus, in some embodiments, bridgeRNA sequence can be provided by providing the nucleotide sequence of LE or RE.

The target site sequence or donor site sequence mediating the excisive recombination or inversion reaction can be located on any polynucleotide sequence of interest, including, but not limited to genomic DNA and plasmids. In some embodiments, the target site sequence and/or donor site sequence is a polynucleotide sequence present in the genome or DNA of interest. In some embodiments the target site sequence and/or donor site sequence naturally occurs in the genome or DNA of interest. In some embodiments, the target site sequence and/or donor site sequence is introduced into the genome or a DNA of interest. Methods of introducing DNA sequences, such as the target site sequence or donor site sequence, into a genome or DNA of interest are known in the art and include, but are not limited to, CRISPR-Cas9, homology directed repair (HDR), transposases, integrases, etc. In some embodiments, the genomic DNA is located in a cell. In some embodiments, the cell is a eukaryotic cell. In some embodiments, the cell is prokaryotic. In some embodiments, the cell is a mammalian cell. In some embodiments, the cell is a human cell. In some embodiments, the cell is a mouse cell. In some embodiments, the cell is a stem cell.

The target site sequence may include a transposon left flank (LF) and transposon right flank (RF). The LF or RF sequences may be endogenous sequences for the IS110 used or may be heterologous sequences recognizable by the IS110 used, or may be synthetic sequences that comprise a sequence or structure feature recognized by the IS110 and sufficient to allow insertion of the polynucleotide into the donor site. In certain example embodiments, the LF or RF sequences are truncated. In certain example embodiments, the LF or RF sequences may be between 20-500 base pairs, between 500-490 base pairs, between 500-480 base pairs, between 500-470 base pairs, between 500-460 base pairs, between 500-450 base pairs, between 500-440 base pairs, between 500-430 base pairs, between 500-420 base pairs, between 500-410 base pairs, between 500-400 base pairs, between 400-390 base pairs, between 400-380 base pairs, between 400-370 base pairs, between 400-360 base pairs, between 400-350 base pairs, between 400-340 base pairs, between 400-330 base pairs, between 400-320 base pairs, between 400-310 base pairs, between 400-300 base pairs, between 300-290 base pairs, between 300-280 base pairs, between 300-270 base pairs, between 300-260 base pairs, between 300-250 base pairs, between 300-240 base pairs, between 300-230 base pairs, between 300-220 base pairs, between 300-210 base pairs, between 300-200 base pairs, between 200-100 base pairs, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length. In some embodiments, the target site sequence may include a transposon LT and transposon RT.

The donor site sequence may include a transposon left end (LE) and transposon right end (RE). The LE or RE sequences may be endogenous sequences for the IS110 used or may be heterologous sequences recognizable by the IS110 used, or may be synthetic sequences that comprise a sequence or structure feature recognized by the IS110 and sufficient to allow insertion of the polynucleotide into the donor site. In certain example embodiments, the LE or RE sequences are truncated. In certain example embodiments, the LE or RE sequences may be between 20-500 base pairs, between 500-490 base pairs, between 500-480 base pairs, between 500-470 base pairs, between 500-460 base pairs, between 500-450 base pairs, between 500-440 base pairs, between 500-430 base pairs, between 500-420 base pairs, between 500-410 base pairs, between 500-400 base pairs, between 400-390 base pairs, between 400-380 base pairs, between 400-370 base pairs, between 400-360 base pairs, between 400-350 base pairs, between 400-340 base pairs, between 400-330 base pairs, between 400-320 base pairs, between 400-310 base pairs, between 400-300 base pairs, between 300-290 base pairs, between 300-280 base pairs, between 300-270 base pairs, between 300-260 base pairs, between 300-250 base pairs, between 300-240 base pairs, between 300-230 base pairs, between 300-220 base pairs, between 300-210 base pairs, between 300-200 base pairs, between 200-100 base pairs, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length. In some embodiments, the donor site sequence may include a transposon LD and transposon RD.

E.3. Non-Core bridgeRNA Reprogramming

Disclosed herein is exemplary reprogramming of the target binding loop of a bridgeRNA in IS110 family transposase embodiments that dₒ not use a core sequence. Any target site sequence can be targeted for transposition by reprogramming the nucleotide sequences of LTG and RTG so they are complementary to the target site sequence of interest. For example, in order to target the target site sequence X₁X₂X₃X₄X₅X₆X₇X₈X₉X₁₀X₁₁ where X is any nucleotide, and X₈ is the 3′ nucleotide of LT and X₉ is 5′ nucleotide of RT and n is zero (i.e., Xₙ is absent and X₈X₉ is the concatenation site between LT and RT) or comprises 1 to 2 nucleotides, the bridgeRNA encodes an LTG in the 5′ to 3′ direction X₁X₂X₃X₄X₅X₆X₇X₈ and an RTG in the 5′ to 3′ direction Y₁₁Y₁₀Y₉ where Y is the complementary nucleotide to X. In some embodiments the target site sequence extends beyond X₁₁ by one or more nucleotides, (e.g., by one nucleotide to X₁X₂X₃X₄X₅X₆X₇X₈X₉X₁₀X₁₁X₁₂, by two nucleotides to X₁X₂X₃X₄X₅X₆X₇X₈X₉X₁₀X₁₁X₁₂X₁₃, by three nucleotides to X₁X₂X₃X₄X₅X₆X₇X₈X₉X₁₀X₁₁X₁₂X₁₃X₁₄, by four nucleotides to X₁X₂X₃X₄X₅X₆X₇X₈X₉X₁₀X₁₁X₁₂X₁₃X₁₄X₁₅, etc.) and the bridgeRNA encodes an RTG in the 5′ to 3′ direction Y₁₂Y₁₁Y₁₀Y₉, Y₁₃Y₁₂Y₁₁Y₁₀Y₉, Y₁₄Y₁₃Y₁₂Y₁₁Y₁₀Y₉, or Y₁₅Y₁₄Y₁₃Y₁₂Y₁₁Y₁₀Y₉, etc., where Y is the complementary nucleotide to X. In some embodiments the target site sequence extends in the other direction beyond X₁ by one or more nucleotides, (e.g., by one nucleotide to X₋₁X₁X₂X₃X₄X₅X₆X₇X₈X₉X₁₀X₁₁X₁₂, etc.) and the bridgeRNA encodes an LTG in the 5′ to 3′ direction X₋₁X₁X₂X₃X₄X₅X₆X₇X₈ etc. In some embodiments, the LTG or RTG can be extended further to increase the length of homology to their respective target site sequences. In some embodiments, Xₙ is absent (i.e., X₈X₉ is the concatenation site between RT and LT). In some embodiments, X₁ is one nucleotide. In some embodiments, X₁ is two nucleotides. In some embodiments, the LTG and/or RTG are partially complementary to their respective target site sequences of the target DNA, i.e., there is non-canonical base pairing, mismatch and/or non-contiguous tolerance. In some embodiments, there is a single mismatch or non-canonical base pair in the LTG or RTG nucleotide sequence, e.g., one of X₁X₂X₃X₄X₅X₆X₇X₈ or [Y₁₄Y₁₃Y₁₂ (if present)]Y₁₁Y₁₀Y₉ comprises a mismatch or non-canonical base pair with the target site sequence. In some embodiments, X₄ of the LTG non-canonically base pairs with the target site sequence (e.g., X₄ of the LTG is a “G” which base pairs with a “T” in the target site sequence. In some embodiments, there are two, three or four mismatches or non-canonical base pairs which can be in the LTG, in the RTG, or spread across the LTG and RTG, e.g., two, three, or four of X₁X₂X₃X₄X₅X₆X₇X₈ or [Y₁₄Y₁₃Y₁₂ (if present)]Y₁₁Y₁₀Y₉ comprises a mismatch or non-canonical base pair with the target site sequence. In some embodiments, the two, three or four mismatches or non-canonical base pairs are contiguous. In some embodiments, the two, three or four mismatches or non-canonical base pairs are non-contiguous. In some embodiments, the LTG and/or RTG are partially complementary to their respective target site sequences of the target DNA so that there are one, two, three, or four nucleotides dispersed within LTG and/or RTG that do not base pair with the target DNA and form bulges. A person of skill in the art understands that in RNA “T” is “U.” Although the target site sequence is exemplified as 11 nucleotides in length, in some embodiments, the target site sequence may be shorter or longer. In some embodiments, the target site sequence length is 5 nucleotides, 6 nucleotides, 7 nucleotides, 8 nucleotides, 9 nucleotides, 10 nucleotide, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. The length of LTG and RTG can accordingly vary with the target site sequence length. In some embodiments, the non-canonical base pairing is non-Watson-Crick base pairing (i.e., is not a G-C base pair or A-T/U base pair). In some embodiments, the non-canonical base pairing is wobble base pairing. In some embodiments, the non-canonical base pairing is Hoogsteen base pairing. In some embodiments, the non-canonical base pairing comprises a rG-dT base pair, a rU-dG base pair, a rA-dC base pair, a rC-dA base pair, a rA-dG base pair, or a rG-dG base pair.

In some embodiments, disclosed herein are three bridgeRNA reprogramming approaches that can increase the specificity and/or efficiency of the system. First, in some embodiments, the bridgeRNA is reprogrammed so that LTG and/or RTG have longer homology to the target site sequence. In this embodiment, the target binding loop sequence itself is not made longer but the nucleotide sequence is reprogrammed so that additional nucleotide bases of the target binding loop sequence of the bridgeRNA are complementary to the target site sequence. For example, a bridgeRNA that has a 9 base LTG that is complementary to the target site sequence can be reprogrammed so that additional bases of bridgeRNA are complementary to the target site sequence resulting in an LTG with longer homology to the target site sequence. In some embodiments, the LTG comprises at least 7 bases, 8 bases, 9 bases, 10 bases, 15 bases, 20 bases, etc. in length. In some embodiments, the LTG further comprises at least one nucleotide complementary to a nucleotide of the concatenation site of the target site sequence. In some embodiments, the LTG comprises 7 bases complementary to the target site sequence before the concatenation site in the 5′ to 3′ direction. In some embodiments, the LTG comprises 8 bases complementary to the target site sequence before the concatenation site in the 5′ to 3′ direction. In some embodiments, the LTG comprises 9 bases complementary to the target site sequence before the concatenation site in the 5′ to 3′ direction. For example, a bridgeRNA that has a 4 base RTG that is complementary to the target site sequence can be reprogrammed so that additional bases of bridgeRNA are complementary to the target site sequence resulting in an RTG with longer homology to the target site sequence. In some embodiments, the RTG comprises at least 2 bases, 3 bases, 4 bases, 5 bases, 6 bases, 7 bases, 9 bases, 10 bases, 15 bases, 20 bases, etc. in length. In some embodiments, the RTG further comprises at least one nucleotide complementary to a nucleotide of the concatenation site. In some embodiments, the RTG comprises 3 bases complementary to the target site sequence after the concatenation site in the 5′ to 3′ direction. In some embodiments, the RTG comprises 4 bases complementary to the target site sequence after the concatenation site in the 5′ to 3′ direction. In some embodiments, the RTG comprises 5 bases complementary to the target site sequence after the concatenation site in the 5′ to 3′ direction. In some embodiments, the RTG comprises 6 bases complementary to the target site sequence after the concatenation site in the 5′ to 3′ direction. In some embodiments, the RTG comprises 7 bases complementary to the target site sequence after the concatenation site in the 5′ to 3′ direction.

Second, in some embodiments, the bridgeRNA is reprogrammed so that LDG and/or RDG are shifted relative to each other so that the span of bases of the donor site sequence bound by the LDG and RDG is longer. In this embodiment, the donor binding loop sequence itself is not made longer but the nucleotide sequence is reprogrammed so that LDG and/or RDG are complementary to the donor site sequence so that they span a longer sequence, e.g. overlap between where LDG and RDG bind is reduced. For example, a bridgeRNA that has a LDG X₁X₂X₃X₄X₅X₆X₇X₈ and an RDG Y₁₁Y₁₀Y₉ that targets the donor site sequence X₁X₂X₃X₄X₅X₆X₇X₈X₉X₁₀X₁₁ can be reprogrammed so that the LDG comprises X₋₁X₁X₂X₃X₄X₅X₆X₇ which in conjunction with the RDG Y₁₁Y₁₀Y₉ now targets the donor site sequence X₋₁X₁X₂X₃X₄X₅X₆X₇X₈X₉X₁₀X₁₁ thus increasing the span of bases of the donor site sequence bound by the LDG and RDG. In some embodiments, the LDG can be shifted at least 1 base, 2 bases, 3 bases, etc. in length. In another example, a bridgeRNA that has a LDG X₁X₂X₃X₄X₅X₆X₇X₈ and an RDG Y₁₁Y₁₀Y₉ that targets the donor site sequence X₁X₂X₃X₄X₅X₆X₇X₈X₉X₁₀X₁₁ can be reprogrammed so that the RDG comprises Y₁₂Y₁₁Y₁₀ which in conjunction with the LDG X₁X₂X₃X₄X₅X₆X₇X₈ now targets the target site sequence X₁X₂X₃X₄X₅X₆X₇X₈X₉X₁₀X₁₁X₁₂ thus increasing the span of bases of the target site sequence bound by the LDG and RDG. In some embodiments, the RDG can be shifted at least 1 base, 2 bases, 3 bases, etc. in length. In some embodiments, both the LDG and RDG can be shifted.

Third, in some embodiments, the bridgeRNA is reprogrammed so that LTG and/or RTG have longer homology to the target site sequence. In this embodiment, the target binding loop sequence is made longer to include additional nucleotide bases of the target binding loop sequence of the bridgeRNA that are complementary to the target site sequence. For example, a bridgeRNA that has a 9 base LTG that is complementary to the target site sequence can be reprogrammed to insert additional bases into the bridgeRNA that are complementary to the target site sequence so that the LTG has longer homology to the target site sequence. In some embodiments, the LTG comprises at least 7 bases, 8 bases, 9 bases, 10 bases, 15 bases, 20 bases, etc. in length. In some embodiments, the LTG further comprises at least one nucleotide complementary to a nucleotide of the concatenation site. In some embodiments, the LTG comprises 7 bases complementary to the target site sequence before the concatenation site in the 5′ to 3′ direction. In some embodiments, the LTG comprises 8 bases complementary to the target site sequence before the concatenation site in the 5′ to 3′ direction. In some embodiments, the LTG comprises 9 bases complementary to the target site sequence before the concatenation site in the 5′ to 3′ direction. For example, a bridgeRNA that has a 4 base RTG that is complementary to the target site sequence can be reprogrammed to insert additional bases into the bridgeRNA that are complementary to the target site sequence so that the RTG has longer homology to the target site sequence. In some embodiments, the RTG comprises at least 2 bases, 3 bases, 4 bases, 5 bases, 6 bases, 7 bases, 9 bases, 10 bases, 15 bases, 20 bases, etc. in length. In some embodiments, the RTG further comprises at least one nucleotide complementary to a nucleotide of the concatenation site. In some embodiments, the RTG comprises 3 bases complementary to the target site sequence after the concatenation site in the 5′ to 3′ direction. In some embodiments, the RTG comprises 4 bases complementary to the target site sequence after the concatenation site in the 5′ to 3′ direction. In some embodiments, the RTG comprises 5 bases complementary to the target site sequence after the concatenation site in the 5′ to 3′ direction. In some embodiments, the RTG comprises 6 bases complementary to the target site sequence after the concatenation site in the 5′ to 3′ direction. In some embodiments, the RTG comprises 7 bases complementary to the target site sequence after the concatenation site in the 5′ to 3′ direction.

Disclosed herein is exemplary reprogramming of the donor binding loop of a bridgeRNA in IS110 family transposase embodiments that do not use a core sequence. Any donor site sequence can be targeted for insertion by reprogramming the nucleotide sequences of LDG and RDG so they are complementary to the donor site sequence of interest. For example, in order to target the donor site sequence STIR-Xₙ₁—X₁X₂X₃X₄X₅X₆X₇X₈X₉X₁₀X₁₁—Xₙ₂-STIR where X is any nucleotide and STIR is optional, but if present is a sub-terminal inverted repeat comprising 2 to 20 nucleotides (e.g., such as a three nucleotide STIR “ATA” and “TAT”), X₈ is the 3′ nucleotide of LD and X₉ is 5′ nucleotide of RD and n is zero (i.e., Xₙ is absent and X₈X₉ is the concatenation site between LD and RD) or comprises 1 to 2 nucleotides, and n1 and n2 can independently be zero to 10, the bridgeRNA encodes an LDG in the 5′ to 3′ direction X₁X₂X₃X₄X₅X₆X₇X₈ and an RDG in the 5′ to 3′ direction Y₁₁Y₁₀Y₉ where Y is the complementary nucleotide to X. In some embodiments the donor site sequence extends beyond X₁₁ by one or more nucleotides, (e.g., by one nucleotide to X₁X₂X₃X₄X₅X₆X₇X₈X₉X₁₀X₁₁X₁₂, by two nucleotides to X₁X₂X₃X₄X₅X₆X₇X₈X₉X₁₀X₁₁X₁₂X₁₃, by three nucleotides to X₁X₂X₃X₄X₅X₆X₇X₈X₉X₁₀X₁X₁₂X₁₃X₁₄, by four nucleotides to X₁X₂X₃X₄X₅X₆X₇X₈X₉X₁₀X₁₁X₁₂X₁₃X₁₄X₁₅, etc.) and the bridgeRNA encodes an RDG in the 5′ to 3′ direction Y₁₂Y₁₁Y₁₀Y₉, Y₁₃Y₁₂Y₁₁Y₁₀Y₉, Y₁₄Y₁₃Y₁₂Y₁₁Y₁₀Y₉, or Y₁₅Y₁₄Y₁₃Y₁₂Y₁₁Y₁₀Y₉, where Y is the complementary nucleotide to X. In some embodiments the donor site sequence extends in the other direction beyond X₁ by one or more nucleotides, (e.g., by one nucleotide to X₋₁X₁X₂X₃X₄X₅X₆X₇X₈X₉X₁₀X₁X₁₂, etc.) and the bridgeRNA encodes an LDG in the 5′ to 3′ direction X₋₁X₁X₂X₃X₄X₅X₆X₇X₈ etc. In some embodiments, the LDG or RDG can be extended further to increase the length of homology to their respective target site sequences. In some embodiments, X₁ is absent (i.e., X₈X₉ is the concatenation site between RD and LD). In some embodiments, X₁ is one nucleotide. In some embodiments, X₁ is two nucleotides. In some embodiments, the LDG and/or RDG are partially complementary to their respective donor site sequences of the donor DNA, i.e., there is non-canonical base pairing, mismatch and/or non-contiguous tolerance. In some embodiments, there is a single mismatch or non-canonical base pair in the LDG or RDG nucleotide sequence, e.g., one of X₁X₂X₃X₄X₅X₆X₇X₈ or [Y₁₄Y₁₃Y₁₂ (if present)]Y₁₁Y₁₀Y₉ comprises a mismatch or non-canonical base pair with the donor site sequence. In some embodiments, Y₁₃ of the RDG non-canonically base pairs with the target site sequence (e.g., Y₁₃ of the RDG is a “G” which base pairs with a “T” in the target site sequence). In some embodiments, Y₁₂ of the RDG non-canonically base pairs with the target site sequence (e.g., Y₁₂ of the RDG is a “G” which base pairs with a “T” in the target site sequence). In some embodiments, the nucleotides of the donor site sequence may show a sequence preference. In some embodiments, there are two, three or four mismatches or non-canonical base pair which can be in the LDG, in the RDG, or spread across the LDG and RDG, e.g., two, three, or four of X₁X₂X₃X₄X₅X₆X₇X₈ or [Y₁₄Y₁₃Y₁₂ (if present)]Y₁₁Y₁₀Y₉ comprises a mismatch or non-canonical base pair with the donor site sequence. In some embodiments, Y₁₄Y₁₃ comprise high mismatch tolerance with the donor site sequence. In some embodiments, the two, three or four mismatches or non-canonical base pairs are contiguous. In some embodiments, the two, three or four mismatches or non-canonical base pairs are non-contiguous. In some embodiments, the LDG and/or RDG are partially complementary to their respective donor site sequences of the donor DNA so that there are one, two, three, or four nucleotides dispersed within LDG and/or RDG that do not base pair with the donor DNA and form bulges (see e.g., FIG. 36A). A person of skill in the art understands that in RNA “T” is “U.” Although the donor site sequence between STIR-Xₙ₁ and Xₙ₂-STIR is exemplified as 11 nucleotides in length, in some embodiments, the donor site sequence between STIR-Xₙ₁ and Xₙ₂-STIR may be shorter or longer. In some embodiments, the donor site length between STIR-Xₙ₁ and Xₙ₂-STIR is 5 nucleotides, 6 nucleotides, 7 nucleotides, 8 nucleotides, 9 nucleotides, 10 nucleotide, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. The length of LDG and RDG can accordingly vary with the donor site length. In some embodiments, the STIR, if present, comprises a G/T rich nucleotide sequence. In some embodiments, the 5′ STIR, if present, comprises a G/T rich nucleotide sequence. In some embodiments, the non-canonical base pairing is non-Watson-Crick base pairing (i.e., is not a G-C base pair or A-T/U base pair). In some embodiments, the non-canonical base pairing is wobble base pairing. In some embodiments, the non-canonical base pairing is Hoogsteen base pairing. In some embodiments, the non-canonical base pairing comprises a rG-dT base pair, a rU-dG base pair, a rA-dC base pair, a rC-dA base pair, a rA-dG base pair, or a rG-dG base pair.

In some embodiments, disclosed herein are three bridgeRNA reprogramming approaches that can increase the specificity and/or efficiency of the system. First, in some embodiments, the bridgeRNA is reprogrammed so that LDG and/or RDG have longer homology to the donor site sequence. In this embodiment, the donor binding loop sequence itself is not made longer but the nucleotide sequence is reprogrammed so that additional nucleotide bases of the donor binding loop sequence of the bridgeRNA are complementary to the donor site sequence. For example, a bridgeRNA that has a 9 base LDG that is complementary to the donor site sequence can be reprogrammed so that additional bases of bridgeRNA are complementary to the donor site sequence resulting in an LDG with longer homology to the donor site sequence. In some embodiments, the LTG comprises at least 7 bases, 8 bases, 9 bases, 10 bases, 15 bases, 20 bases, etc. in length. In some embodiments, the LDG further comprises at least one nucleotide complementary to a nucleotide of the concatenation site of the donor site sequence. In some embodiments, the LDG comprises 7 bases complementary to the donor site sequence before the concatenation site in the 5′ to 3′ direction. In some embodiments, the LDG comprises 8 bases complementary to the donor site sequence before the concatenation site in the 5′ to 3′ direction. In some embodiments, the LDG comprises 9 bases complementary to the donor site sequence before the concatenation site in the 5′ to 3′ direction. For example, a bridgeRNA that has a 4 base RDG that is complementary to the donor site sequence can be reprogrammed so that additional bases of bridgeRNA are complementary to the donor site sequence resulting in an RDG with longer homology to the donor site sequence. In some embodiments, the RDG comprises at least 2 bases, 3 bases, 4 bases, 5 bases, 6 bases, 7 bases, 9 bases, 10 bases, 15 bases, 20 bases, etc. in length. In some embodiments, the RDG further comprises at least one nucleotide complementary to a nucleotide of the concatenation site. In some embodiments, the RDG comprises 3 bases complementary to the donor site sequence after the concatenation site in the 5′ to 3′ direction. In some embodiments, the RDG comprises 4 bases complementary to the donor site sequence after the concatenation site in the 5′ to 3′ direction. In some embodiments, the RDG comprises 5 bases complementary to the donor site sequence after the concatenation site in the 5′ to 3′ direction. In some embodiments, the RDG comprises 6 bases complementary to the donor site sequence after the concatenation site in the 5′ to 3′ direction. In some embodiments, the RDG comprises 7 bases complementary to the donor site sequence after the concatenation site in the 5′ to 3′ direction.

Second, in some embodiments, the bridgeRNA is reprogrammed so that LDG and/or RDG are shifted relative to each other so that the span of bases of the donor site sequence bound by the LDG and RDG is longer. In this embodiment, the donor binding loop sequence itself is not made longer but the nucleotide sequence is reprogrammed so that LDG and/or RDG are complementary to the donor site sequence so that they span a longer sequence, e.g. overlap between where LDG and RDG bind is reduced. For example, a bridgeRNA that has a LDG X₁X₂X₃X₄X₅X₆X₇X₈ and an RDG Y₁₁Y₁₀Y₉ that targets the donor site sequence X₁X₂X₃X₄X₅X₆X₇X₈X₉X₁₀X₁₁ can be reprogrammed so that the LDG comprises X₋₁X₁X₂X₃X₄X₅X₆X₇ which in conjunction with the RDG Y₁₁Y₁₀Y₉ now targets the donor site sequence X₋₁X₁X₂X₃X₄X₅X₆X₇X₈X₉X₁₀X₁₁ thus increasing the span of bases of the donor site sequence bound by the LDG and RDG. In some embodiments, the LDG can be shifted at least 1 base, 2 bases, 3 bases, etc. in length. In another example, a bridgeRNA that has a LDG X₁X₂X₃X₄X₅X₆X₇X₈ and an RDG Y₁₁Y₁₀Y₉ that targets the donor site sequence X₁X₂X₃X₄X₅X₆X₇X₈X₉X₁₀X₁₁ can be reprogrammed so that the RDG comprises Y₁₂Y₁₁Y₁₀ which in conjunction with the LDG X₁X₂X₃X₄X₅X₆X₇X₈ now targets the target site sequence X₁X₂X₃X₄X₅X₆X₇X₈X₉X₁₀X₁₁X₁₂ thus increasing the span of bases of the target site sequence bound by the LDG and RDG. In some embodiments, the RDG can be shifted at least 1 base, 2 bases, 3 bases, etc. in length. In some embodiments, both the LDG and RDG can be shifted.

Third, in some embodiments, the bridgeRNA is reprogrammed so that LDG and/or RDG have longer homology to the donor site sequence. In this embodiment, the donor binding loop sequence is made longer to include additional nucleotide bases of the donor binding loop sequence of the bridgeRNA that are complementary to the donor site sequence. For example, a bridgeRNA that has a 9 base LDG that is complementary to the donor site sequence can be reprogrammed to insert additional bases into the bridgeRNA that are complementary to the donor site sequence so that the LDG has longer homology to the target site sequence. In some embodiments, the LDG comprises at least 7 bases, 8 bases, 9 bases, 10 bases, 15 bases, 20 bases, etc. in length. In some embodiments, the LDG further comprises at least one nucleotide complementary to a nucleotide of the concatenation site. In some embodiments, the LDG comprises 7 bases complementary to the donor site sequence before the concatenation site in the 5′ to 3′ direction. In some embodiments, the LDG comprises 8 bases complementary to the donor site sequence before the concatenation site in the 5′ to 3′ direction. In some embodiments, the LDG comprises 9 bases complementary to the donor site sequence before the concatenation site in the 5′ to 3′ direction. For example, a bridgeRNA that has a 4 base RDG that is complementary to the donor site sequence can be reprogrammed to insert additional bases into the bridgeRNA that are complementary to the donor site sequence so that the RDG has longer homology to the target site sequence. In some embodiments, the RDG comprises at least 2 bases, 3 bases, 4 bases, 5 bases, 6 bases, 7 bases, 9 bases, 10 bases, 15 bases, 20 bases, etc. in length. In some embodiments, the RDG further comprises at least one nucleotide complementary to a nucleotide of the concatenation site. In some embodiments, the RDG comprises 3 bases complementary to the donor site sequence after the concatenation site in the 5′ to 3′ direction. In some embodiments, the RDG comprises 4 bases complementary to the donor site sequence after the concatenation site in the 5′ to 3′ direction. In some embodiments, the RDG comprises 5 bases complementary to the donor site sequence after the concatenation site in the 5′ to 3′ direction. In some embodiments, the RDG comprises 6 bases complementary to the donor site sequence after the concatenation site in the 5′ to 3′ direction. In some embodiments, the RDG comprises 7 bases complementary to the donor site sequence after the concatenation site in the 5′ to 3′ direction.

The donor site sequence may refer to the wild-type RE-LE or a subsequence thereof. The donor site sequence may refer to sequences derived or engineered from the wild-type RE-LE or non-coding end sequences, including substitutions, insertions, deletions, and truncations to as few as 5 bases. Subsequences of the donor site base-pair with subsequences of the bridgeRNA found within the donor binding loop. In conjunction with these teachings, the donor site sequence may comprise LD and RD sequences that are complementary, at least in part, to LDG and RDG sequences of a bridgeRNA molecule, respectively. In some embodiments, in the context of formation of an IS110 transpososome complex, the donor site sequence refers to a DNA sequence to which a bridgeRNA sequence is designed to have portions complementary to both strands of the donor site sequence, where base-pairing between a donor site sequence and a bridgeRNA sequence promotes the formation of a IS110 transposition complex.

The target site sequence may refer to the wild-type LF-RF or a subsequence thereof. The target site sequence may refer to sequences derived or engineered from the wild-type LF-RF sequences, including substitutions, insertions, deletions, and truncations to as few as 5 bases. Subsequences of the target site base-pair with subsequences of the bridgeRNA found within the target binding loop. The target site may also be referred to as the “acceptor target,” “acceptor target site,” “target sequence,” or “target site.” In conjunction with these teachings, the target site sequence may comprise LT and RT sequences that are complementary, at least in part, to LTG and RTG sequences of a bridgeRNA molecule, respectively. In some embodiments, in the context of formation of an IS110 transpososome complex, the target site sequence refers to a DNA sequence to which a bridgeRNA sequence is designed to have portions complementarity to both strands of the target site sequence, where base-pairing between a target site sequence and a bridgeRNA sequence promotes the formation of a IS110 transposition complex.

In some embodiments, the 3′ end of the bridgeRNA can further comprise a nucleotide sequence that corresponds to a nucleotide sequence derived from the coding sequence of the recombinase found in the natural IS element. In some embodiments, the 3′ end of the bridgeRNA can further comprise a nucleotide sequence that corresponds to about 100 nucleotides derived from the 5′ end of coding sequence of the recombinase found in the natural IS element. See FIGS. 40A-D. In some embodiments, the 3′ end of the bridgeRNA can further comprise a nucleotide sequence that corresponds to about 100 nucleotides derived from the 5′ end of coding sequence of the recombinase found in the natural IS element (e.g., when bridgeRNA is encoded on the bottom strand of the IS element). In some embodiments, the 5′ end of the bridgeRNA can further comprise a nucleotide sequence that corresponds to a nucleotide sequence derived from the coding sequence of the recombinase found in the natural IS element. In some embodiments, the 5′ end of the bridgeRNA can further comprise a nucleotide sequence that corresponds to about 100 nucleotides derived from the 3′ end of coding sequence of the recombinase found in the natural IS element.

E.4. Core bridgeRNA Reprogramming

Disclosed herein is exemplary reprogramming of the target binding loop of a bridgeRNA in IS110 family transposase embodiments that use a core sequence. Exemplary reprogramming of bridgeRNA is shown in FIG. 4. Any target site can be targeted for insertion by reprogramming the nucleotide sequences of LTG and RTG so they are complementary to the target site sequence of interest. For example, in order to target the target site sequence X₁X₂X₃X₄X₅X₆X₇X₈X₉X₁₀X₁₁ where X is any nucleotide, and X₈X₉ are the core, the bridgeRNA encodes an LTG in the 5′ to 3′ direction X₁X₂X₃X₄X₅X₆X₇X₈ and an RTG in the 5′ to 3′ direction Y₁₁Y₁₀Y₉Y₈ where Y is the complementary nucleotide to X. In some embodiments, the bridgeRNA encodes an LTG in the 5′ to 3′ direction X₁X₂X₃X₄X₅X₆X₇X₈X₉. In some embodiments, the bridgeRNA encodes an RTG in the 5′ to 3′ direction Y₁₁Y₁₀Y₉ where Y is the complementary nucleotide to X. In some embodiments, the bridgeRNA encodes an LTG in the 5′ to 3′ direction X₁X₂X₃X₄X₅X₆X₇. In some embodiments, the bridgeRNA encodes an RTG in the 5′ to 3′ direction Y₁Y₁₀ where Y is the complementary nucleotide to X. In some embodiments the target site sequence extends beyond X₁₁ by one or more nucleotides, (e.g., by one nucleotide to X₁X₂X₃X₄X₅X₆X₇X₈X₉X₁₀X₁₁X₁₂, by two nucleotides to X₁X₂X₃X₄X₅X₆X₇X₈X₉X₁₀X₁₁X₁₂X₁₃, by three nucleotides to X₁X₂X₃X₄X₅X₆X₇X₈X₉X₁₀X₁₁X₁₂X₁₃X₁₄, by four nucleotides to X₁X₂X₃X₄X₅X₆X₇X₈X₉X₁₀X₁₁X₁₂X₁₃X₁₄X₁₅, etc.) and the bridgeRNA encodes an RTG in the 5′ to 3′ direction Y₁₂Y₁₁Y₁₀, Y₁₂Y₁₁Y₁₀Y₉, Y₁₂Y₁₁Y₁₀Y₉Y₈, Y₁₃Y₁₂Y₁₁Y₁₀, Y₁₃Y₁₂Y₁₁Y₁₀Y₉, Y₁₃Y₁₂Y₁₁Y₁₀Y₉Y₈, Y₁₄Y₁₃Y₁₂Y₁₁Y₁₀, Y₁₄Y₁₃Y₁₂Y₁₁Y₁₀Y₉, Y₁₄Y₁₃Y₁₂Y₁₁Y₁₀Y₉Y₈, Y₁₅Y₁₄Y₁₃Y₁₂Y₁₁Y₁₀Y₉, or Y₁₅Y₁₄Y₁₃Y₁₂Y₁₁Y₁₀Y₉Y₈, where Y is the complementary nucleotide to X. In some embodiments the target site sequence extends in the other direction beyond X₁ by one or more nucleotides, (e.g., by one nucleotide to X₋₁X₁X₂X₃X₄X₅X₆X₇X₈X₉X₁₀X₁₁X₁₂, etc.) and the bridgeRNA encodes an LTG in the 5′ to 3′ direction X₋₁X₁X₂X₃X₄X₅X₆X₇X₈ or X₋₁X₁X₂X₃X₄X₅X₆X₇X₈X₉ etc. In some embodiments, the LTG or RTG can be extended further to increase the length of homology to their respective target site sequences. In some embodiments, the target site can comprise one or two additional nucleotides between X₇ and X₈ or between X₉ and X₁₀ which are not bound by LTG or RTG. In some embodiments, the core comprises a single nucleotide (i.e., there is either no X₈ or no X₉). In some embodiments, the LTG and/or RTG are partially complementary to their respective target site sequences of the target DNA, i.e., there is non-canonical base pairing, mismatch and/or non-contiguous tolerance. In some embodiments, there is a single mismatch or non-canonical base pair in the LTG or RTG nucleotide sequence, i.e., one of X₁X₂X₃X₄X₅X₆X₇X₈X₉ or [Y₁₄Y₁₃Y₁₂ (if present)]Y₁₁Y₁₀[Y₉Y₈ (if present)] comprises a mismatch or non-canonical base pair with the target site sequence. In some embodiments, X₄ of the LTG non-canonically base pairs with the target site sequence (e.g., X₄ of the LTG is a “G” which base pairs with a “T” in the target site sequence. In some embodiments, there are two, three or four mismatches or non-canonical base pairs which can be in the LTG, in the RTG, or spread across the LTG and RTG, e.g., two, three, or four of X₁X₂X₃X₄X₅X₆X₇X₈X₉ or [Y₁₄Y₁₃Y₁₂ (if present)]Y₁₁Y₁₀[Y₉Y₈ (if present)] comprises a mismatch or non-canonical base pair with the target site sequence. In some embodiments, the two, three or four mismatches or non-canonical base pairs are contiguous. In some embodiments, the two, three or four mismatches or non-canonical base pairs are non-contiguous. In some embodiments, the LTG and/or RTG are partially complementary to their respective target site sequences of the target DNA so that there are one, two, three, or four nucleotides dispersed within LTG and/or RTG that do not base pair with the target DNA and form bulges. A person of skill in the art understands that in RNA “T” is “U.” Although the target site is exemplified as 11 nucleotides in length, in some embodiments, the target site may be shorter or longer. In some embodiments, the target site length is 5 nucleotides, 6 nucleotides, 7 nucleotides, 8 nucleotides, 9 nucleotides, 10 nucleotide, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. The length of LTG and RTG can accordingly vary with the target site length. The core X₈X₉ of the target site sequence of interest is the same as the core X₈X₉ of the donor site sequence. In some embodiments, the non-canonical base pairing is non-Watson-Crick base pairing (i.e., is not a G-C base pair or A-T/U base pair). In some embodiments, the non-canonical base pairing is wobble base pairing. In some embodiments, the non-canonical base pairing is Hoogsteen base pairing. In some embodiments, the non-canonical base pairing comprises a rG-dT base pair, a rU-dG base pair, a rA-dC base pair, a rC-dA base pair, a rA-dG base pair, or a rG-dG base pair.

In some embodiments, disclosed herein are three bridgeRNA reprogramming approaches that can increase the specificity and/or efficiency of the system. First, in some embodiments, the bridgeRNA is reprogrammed so that LTG and/or RTG have longer homology to the target site sequence. In this embodiment, the target binding loop sequence itself is not made longer but the nucleotide sequence is reprogrammed so that additional nucleotide bases of the target binding loop sequence of the bridgeRNA are complementary to the target site sequence. For example, a bridgeRNA that has a 9 base LTG that is complementary to the target site sequence can be reprogrammed so that additional bases of bridgeRNA are complementary to the target site sequence resulting in an LTG with longer homology to the target site sequence. In some embodiments, the LTG comprises at least 7 bases, 8 bases, 9 bases, 10 bases, 15 bases, 20 bases, etc. in length. In some embodiments, the LTG further comprises at least one nucleotide complementary to a nucleotide of the core sequence. In some embodiments, the LTG comprises 7 bases complementary to the target site sequence before the core sequence in the 5′ to 3′ direction. In some embodiments, the LTG comprises 8 bases complementary to the target site sequence before the core sequence in the 5′ to 3′ direction. In some embodiments, the LTG comprises 9 bases complementary to the target site sequence before the core sequence in the 5′ to 3′ direction. For example, a bridgeRNA that has a 4 base RTG that is complementary to the target site sequence can be reprogrammed so that additional bases of bridgeRNA are complementary to the target site sequence resulting in an RTG with longer homology to the target site sequence. In some embodiments, the RTG comprises at least 2 bases, 3 bases, 4 bases, 5 bases, 6 bases, 7 bases, 9 bases, 10 bases, 15 bases, 20 bases, etc. in length. In some embodiments, the RTG further comprises at least one nucleotide complementary to a nucleotide of the core sequence. In some embodiments, the RTG comprises 3 bases complementary to the target site sequence after the core sequence in the 5′ to 3′ direction. In some embodiments, the RTG comprises 4 bases complementary to the target site sequence after the core sequence in the 5′ to 3′ direction. In some embodiments, the RTG comprises 5 bases complementary to the target site sequence after the core sequence in the 5′ to 3′ direction. In some embodiments, the RTG comprises 6 bases complementary to the target site sequence after the core sequence in the 5′ to 3′ direction. In some embodiments, the RTG comprises 7 bases complementary to the target site sequence after the core sequence in the 5′ to 3′ direction.

Second, in some embodiments, the bridgeRNA is reprogrammed so that LTG and/or RTG are shifted relative to each other so that the span of bases of the target site sequence bound by the LTG and RTG is longer. In this embodiment, the target binding loop sequence itself is not made longer but the nucleotide sequence is reprogrammed so that LTG and/or RTG are complementary to the target site sequence so that they span a longer sequence, e.g. overlap between where LTG and RTG bind is reduced. For example, a bridgeRNA that has a LTG X₁X₂X₃X₄X₅X₆X₇X₈ and an RTG Y₁₁Y₁₀Y₉ that targets the target site sequence X₁X₂X₃X₄X₅X₆X₇X₈X₉X₁₀X₁₁ can be reprogrammed so that the LTG comprises X₋₁X₁X₂X₃X₄X₅X₆X₇ which in conjunction with the RTG Y₁₁Y₁₀Y₉ now targets the target site sequence X₋₁X₁X₂X₃X₄X₅X₆X₇X₈X₉X₁₀X₁₁ thus increasing the span of bases of the target site sequence bound by the LTG and RTG. In some embodiments, the LTG can be shifted at least 1 base, 2 bases, 3 bases, etc. in length. In another example, a bridgeRNA that has a LTG X₁X₂X₃X₄X₅X₆X₇X₈ and an RTG Y₁₁Y₁₀Y₉ that targets the target site sequence X₁X₂X₃X₄X₅X₆X₇X₈X₉X₁₀X₁₁ can be reprogrammed so that the RTG comprises Y₁₂Y₁₁Y₁₀ which in conjunction with the LTG X₁X₂X₃X₄X₅X₆X₇X₈ now targets the target site sequence X₁X₂X₃X₄X₅X₆X₇X₈X₉X₁₀X₁₁X₁₂ thus increasing the span of bases of the target site sequence bound by the LTG and RTG. In some embodiments, the RTG can be shifted at least 1 base, 2 bases, 3 bases, etc. in length. In some embodiments, both the LTG and RTG can be shifted.

Third, in some embodiments, the bridgeRNA is reprogrammed so that LTG and/or RTG have longer homology to the target site sequence. In this embodiment, the target binding loop sequence is made longer to include additional nucleotide bases of the target binding loop sequence of the bridgeRNA that are complementary to the target site sequence. For example, a bridgeRNA that has a 9 base LTG that is complementary to the target site sequence can be reprogrammed to insert additional bases into the bridgeRNA that are complementary to the target site sequence so that the LTG has longer homology to the target site sequence. In some embodiments, the LTG comprises at least 7 bases, 8 bases, 9 bases, 10 bases, 15 bases, 20 bases, etc. in length. In some embodiments, the LTG further comprises at least one nucleotide complementary to a nucleotide of the concatenation site. In some embodiments, the LTG comprises 7 bases complementary to the target site sequence before the concatenation site in the 5′ to 3′ direction. In some embodiments, the LTG comprises 8 bases complementary to the target site sequence before the concatenation site in the 5′ to 3′ direction. In some embodiments, the LTG comprises 9 bases complementary to the target site sequence before the concatenation site in the 5′ to 3′ direction. For example, a bridgeRNA that has a 4 base RTG that is complementary to the target site sequence can be reprogrammed to insert additional bases into the bridgeRNA that are complementary to the target site sequence so that the RTG has longer homology to the target site sequence. In some embodiments, the RTG comprises at least 2 bases, 3 bases, 4 bases, 5 bases, 6 bases, 7 bases, 9 bases, 10 bases, 15 bases, 20 bases, etc. in length. In some embodiments, the RTG further comprises at least one nucleotide complementary to a nucleotide of the concatenation site. In some embodiments, the RTG comprises 3 bases complementary to the target site sequence after the concatenation site in the 5′ to 3′ direction. In some embodiments, the RTG comprises 4 bases complementary to the target site sequence after the concatenation site in the 5′ to 3′ direction. In some embodiments, the RTG comprises 5 bases complementary to the target site sequence after the concatenation site in the 5′ to 3′ direction. In some embodiments, the RTG comprises 6 bases complementary to the target site sequence after the concatenation site in the 5′ to 3′ direction. In some embodiments, the RTG comprises 7 bases complementary to the target site sequence after the concatenation site in the 5′ to 3′ direction.

Disclosed herein is exemplary reprogramming of the donor binding loop of a bridgeRNA in IS110 family transposase embodiments that use a core sequence. Any donor site sequence can be targeted for insertion by reprogramming the nucleotide sequences of LDG and RDG so they are complementary to the donor site sequence of interest. For example, in order to target the donor site STIR-Xₙ₁—X₁X₂X₃X₄X₅X₆X₇X₈X₉X₁₀X₁₁—X₁₂-STIR where X is any nucleotide, STIR is optional, but if present is a sub-terminal inverted repeat comprising 2 to 20 nucleotides (e.g., such as a three nucleotide STIR “ATA” and “TAT”), and X₈X₉ are the core, and n1 and n2 can independently be zero to 10, the bridgeRNA encodes an LDG in the 5′ to 3′ direction X₁X₂X₃X₄X₅X₆X₇X₈ and an RDG in the 5′ to 3′ direction Y₁₁Y₁₀Y₉Y₈ where Y is the complementary nucleotide to X. In some embodiments, the bridgeRNA encodes an LDG in the 5′ to 3′ direction X₁X₂X₃X₄X₅X₆X₇X₈X₉. In some embodiments, the bridgeRNA encodes an RDG in the 5′ to 3′ direction Y₁₁Y₁₀Y₉ where Y is the complementary nucleotide to X. In some embodiments, the bridgeRNA encodes an LDG in the 5′ to 3′ direction X₁X₂X₃X₄X₅X₆X₇. In some embodiments, the bridgeRNA encodes an RDG in the 5′ to 3′ direction Y₁₁Y₁₀ where Y is the complementary nucleotide to X. In some embodiments the donor site sequence extends beyond X₁₁ by one or more nucleotides, (e.g., by one nucleotide to X₁X₂X₃X₄X₅X₆X₇X₈X₉X₁₀X₁₁X₁₂, by two nucleotides to X₁X₂X₃X₄X₅X₆X₇X₈X₉X₁₀X₁₁X₁₂X₁₃, by three nucleotides to X₁X₂X₃X₄X₅X₆X₇X₈X₉X₁₀X₁₁X₁₂X₁₃X₁₄, by four nucleotides to X₁X₂X₃X₄X₅X₆X₇X₈X₉X₁₀X₁₁X₁₂X₁₃X₁₄X₁₅, etc.) and the bridgeRNA encodes an RDG in the 5′ to 3′ direction Y₁₂Y₁₁Y₁₀, Y₁₂Y₁₁Y₁₀Y₉, Y₁₂Y₁₁Y₁₀Y₉Y₈, Y₁₃Y₁₂Y₁₁Y₁₀, Y₁₃Y₁₂Y₁₁Y₁₀Y₉, Y₁₃Y₁₂Y₁₁Y₁₀Y₉Y₈, Y₁₄Y₁₃Y₁₂Y₁₁Y₁₀, Y₁₄Y₁₃Y₁₂Y₁₁Y₁₀Y₉, Y₁₄Y₁₃Y₁₂Y₁₁Y₁₀Y₉Y₈, Y₁₅Y₁₄Y₁₃Y₁₂Y₁₁Y₁₀Y₉, or Y₁₅Y₁₄Y₁₃Y₁₂Y₁₁Y₁₀Y₉Y₈, where Y is the complementary nucleotide to X. In some embodiments the donor site sequence extends in the other direction beyond X₁ by one or more nucleotides, (e.g., by one nucleotide to X₋₁X₁X₂X₃X₄X₅X₆X₇X₈X₉X₁₀X₁₁X₁₂, etc.) and the bridgeRNA encodes an LDG in the 5′ to 3′ direction X₋₁X₁X₂X₃X₄X₅X₆X₇X₈ etc. In some embodiments, the LDG or RDG can be extended further to increase the length of homology to their respective target site sequence. In some embodiments, the donor site can comprise one or two additional nucleotides between X₇ and X₈ or between X₉ and X₁₀ which are not bound by LDG or RDG. In some embodiments, the core comprises a single nucleotide (i.e., there is either no X₈ or no X₉). A person of skill in the art understands that in RNA “T” is “U.” In some embodiments, the LDG and/or RDG are partially complementary to their respective donor site sequences of the donor DNA, i.e., there is non-canonical base pairing, mismatch and/or non-contiguous tolerance. In some embodiments, there is a single mismatch in the LDG or RDG nucleotide sequence, e.g., one of X₁X₂X₃X₄X₅X₆X₇X₈ or [Y₁₄Y₁₃Y₁₂ (if present)]Y₁₁Y₁₀[Y₉Y₈ (if present)] comprises a mismatch or non-canonical base pair with the donor site sequence. In some embodiments, Y₁₃ of the RDG non-canonically base pairs with the target site sequence (e.g., Y₁₃ of the RDG is a “G” which base pairs with a “T” in the target site sequence). In some embodiments, Y₁₂ of the RDG non-canonically base pairs with the target site sequence (e.g., Y₁₂ of the RDG is a “G” which base pairs with a “T” in the target site sequence). In some embodiments, the nucleotides of the donor site sequence may show a sequence preference. In some embodiments, there are two, three or four mismatches or non-canonical base pairs which can be in the LDG, in the RDG, or spread across the LDG and RDG, e.g., two, three, or four of X₁X₂X₃X₄X₅X₆X₇X₈ or [Y₁₄Y₁₃Y₁₂ (if present)]Y₁₁Y₁₀[Y₉Y₈ (if present)] comprises a mismatch or non-canonical base pair with the donor site sequence. In some embodiments, Y₁₄Y₁₃ comprise high mismatch tolerance with the donor site sequence. In some embodiments, the two, three or four mismatches or non-canonical base pairs are contiguous. In some embodiments, the two, three or four mismatches or non-canonical base pairs are non-contiguous. In some embodiments, Y₁₄Y₁₃ comprise high mismatch tolerance with the donor site sequence. In some embodiments, the LDG and/or RDG are partially complementary to their respective donor site sequences of the donor DNA so that there are one, two, three, or four nucleotides dispersed within LDG and/or RDG that dₒ not base pair with the donor DNA and form bulges (see e.g., FIG. 36A). Although the donor site sequence between STIR-Xₙ₁ and Xₙ₂-STIR is exemplified as 11 nucleotides in length, in some embodiments, the donor site between the STIRs may be shorter or longer. In some embodiments, the target site length is 5 nucleotides, 6 nucleotides, 7 nucleotides, 8 nucleotides, 9 nucleotides, 10 nucleotide, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. The length of LDG and RDG can accordingly vary with the donor site length. The core X₈X₉ of the donor site sequence of interest is the same as the core X₈X₉ of the target site sequence. In some embodiments, the STIR, if present, comprises a G/T rich nucleotide sequence. In some embodiments, the 5′ STIR, if present, comprises a G/T rich nucleotide sequence. In some embodiments, the non-canonical base pairing is non-Watson-Crick base pairing (i.e., is not a G-C base pair or A-T/U base pair). In some embodiments, the non-canonical base pairing is wobble base pairing. In some embodiments, the non-canonical base pairing is Hoogsteen base pairing. In some embodiments, the non-canonical base pairing comprises a rG-dT base pair, a rU-dG base pair, a rA-dC base pair, a rC-dA base pair, a rA-dG base pair, or a rG-dG base pair.

In some embodiments, disclosed herein are three bridgeRNA reprogramming approaches that can increase the specificity and/or efficiency of the system. First, in some embodiments, the bridgeRNA is reprogrammed so that LDG and/or RDG have longer homology to the donor site sequence. In this embodiment, the donor binding loop sequence itself is not made longer but the nucleotide sequence is reprogrammed so that additional nucleotide bases of the donor binding loop sequence of the bridgeRNA are complementary to the donor site sequence. For example, a bridgeRNA that has a 9 base LDG that is complementary to the donor site sequence can be reprogrammed so that additional bases of bridgeRNA are complementary to the donor site sequence resulting in an LDG with longer homology to the donor site sequence. In some embodiments, the LTG comprises at least 7 bases, 8 bases, 9 bases, 10 bases, 15 bases, 20 bases, etc. in length. In some embodiments, the LDG further comprises at least one nucleotide complementary to a nucleotide of the core sequence of the donor site sequence. In some embodiments, the LDG comprises 7 bases complementary to the donor site sequence before the core sequence in the 5′ to 3′ direction. In some embodiments, the LDG comprises 8 bases complementary to the donor site sequence before the core sequence in the 5′ to 3′ direction. In some embodiments, the LDG comprises 9 bases complementary to the donor site sequence before the core sequence in the 5′ to 3′ direction. For example, a bridgeRNA that has a 4 base RDG that is complementary to the donor site sequence can be reprogrammed so that additional bases of bridgeRNA are complementary to the donor site sequence resulting in an RDG with longer homology to the donor site sequence. In some embodiments, the RDG comprises at least 2 bases, 3 bases, 4 bases, 5 bases, 6 bases, 7 bases, 9 bases, 10 bases, 15 bases, 20 bases, etc. in length. In some embodiments, the RDG further comprises at least one nucleotide complementary to a nucleotide of the core sequence. In some embodiments, the RDG comprises 3 bases complementary to the donor site sequence after the core sequence in the 5′ to 3′ direction. In some embodiments, the RDG comprises 4 bases complementary to the donor site sequence after the core sequence in the 5′ to 3′ direction. In some embodiments, the RDG comprises 5 bases complementary to the donor site sequence after the core sequence in the 5′ to 3′ direction. In some embodiments, the RDG comprises 6 bases complementary to the donor site sequence after the core sequence in the 5′ to 3′ direction. In some embodiments, the RDG comprises 7 bases complementary to the donor site sequence after the core sequence in the 5′ to 3′ direction.

Second, in some embodiments, the bridgeRNA is reprogrammed so that LDG and/or RDG are shifted relative to each other so that the span of bases of the donor site sequence bound by the LDG and RDG is longer. In this embodiment, the donor binding loop sequence itself is not made longer but the nucleotide sequence is reprogrammed so that LDG and/or RDG are complementary to the donor site sequence so that they span a longer sequence, e.g., overlap between where LDG and RDG bind is reduced. For example, a bridgeRNA that has a LDG X₁X₂X₃X₄X₅X₆X₇X₈ and an RDG Y₁₁Y₁₀Y₉ that targets the donor site sequence X₁X₂X₃X₄X₅X₆X₇X₈X₉X₁₀X₁₁ can be reprogrammed so that the LDG comprises X₋₁X₁X₂X₃X₄X₅X₆X₇ which in conjunction with the RDG Y₁₁Y₁₀Y₉ now targets the donor site sequence X₋₁X₁X₂X₃X₄X₅X₆X₇X₈X₉X₁₀X₁₁ thus increasing the span of bases of the donor site sequence bound by the LDG and RDG. In some embodiments, the LDG can be shifted at least 1 base, 2 bases, 3 bases, etc. in length. In another example, a bridgeRNA that has a LDG X₁X₂X₃X₄X₅X₆X₇X₈ and an RDG Y₁₁Y₁₀Y₉ that targets the donor site sequence X₁X₂X₃X₄X₅X₆X₇X₈X₉X₁₀X₁₁ can be reprogrammed so that the RDG comprises Y₁₂Y₁₁Y₁₀ which in conjunction with the LDG X₁X₂X₃X₄X₅X₆X₇X₈ now targets the target site sequence X₁X₂X₃X₄X₅X₆X₇X₈X₉X₁₀X₁X₁₂ thus increasing the span of bases of the target site sequence bound by the LDG and RDG. In some embodiments, the RDG can be shifted at least 1 base, 2 bases, 3 bases, etc. in length. In some embodiments, both the LDG and RDG can be shifted.

Third, in some embodiments, the bridgeRNA is reprogrammed so that LDG and/or RDG have longer homology to the donor site sequence. In this embodiment, the donor binding loop sequence is made longer to include additional nucleotide bases of the donor binding loop sequence of the bridgeRNA that are complementary to the donor site sequence. For example, a bridgeRNA that has a 9 base LDG that is complementary to the donor site sequence can be reprogrammed to insert additional bases into the bridgeRNA that are complementary to the donor site sequence so that the LDG has longer homology to the target site sequence. In some embodiments, the LDG comprises at least 7 bases, 8 bases, 9 bases, 10 bases, 15 bases, 20 bases, etc. in length. In some embodiments, the LDG further comprises at least one nucleotide complementary to a nucleotide of the core sequence. In some embodiments, the LDG comprises 7 bases complementary to the donor site sequence before the core sequence in the 5′ to 3′ direction. In some embodiments, the LDG comprises 8 bases complementary to the donor site sequence before the core sequence in the 5′ to 3′ direction. In some embodiments, the LDG comprises 9 bases complementary to the donor site sequence before the core sequence in the 5′ to 3′ direction. For example, a bridgeRNA that has a 4 base RDG that is complementary to the donor site sequence can be reprogrammed to insert additional bases into the bridgeRNA that are complementary to the donor site sequence so that the RDG has longer homology to the target site sequence. In some embodiments, the RDG comprises at least 2 bases, 3 bases, 4 bases, 5 bases, 6 bases, 7 bases, 9 bases, 10 bases, 15 bases, 20 bases, etc. in length. In some embodiments, the RDG further comprises at least one nucleotide complementary to a nucleotide of the core sequence. In some embodiments, the RDG comprises 3 bases complementary to the donor site sequence after the core sequence in the 5′ to 3′ direction. In some embodiments, the RDG comprises 4 bases complementary to the donor site sequence after the core sequence in the 5′ to 3′ direction. In some embodiments, the RDG comprises 5 bases complementary to the donor site sequence after the core sequence in the 5′ to 3′ direction. In some embodiments, the RDG comprises 6 bases complementary to the donor site sequence after the core sequence in the 5′ to 3′ direction. In some embodiments, the RDG comprises 7 bases complementary to the donor site sequence after the core sequence in the 5′ to 3′ direction.

The donor site sequence may refer to the wild-type RE-core-LE or a subsequence thereof. The donor site may refer to sequences derived or engineered from the wild-type RE-core-LE or non-coding end sequences, including substitutions, insertions, deletions, and truncations to as few as 5 bases. Subsequences of the donor site sequence base-pair with subsequences of the bridgeRNA found within the donor binding loop. In conjunction with these teachings, the donor site sequence may comprise LD and RD sequences that are complementary, at least in part, to LDG and RDG sequences of a bridgeRNA molecule, respectively. In some embodiments, in the context of formation of an IS110 transpososome complex, the donor site sequence refers to a DNA sequence to which a bridgeRNA sequence is designed to have portions complementarity to both strands of the donor site sequence, where base-pairing between a donor site sequence and a bridgeRNA sequence promotes the formation of a IS110 transposition complex.

The target site sequence may refer to the wild-type LF-RF or a subsequence thereof. The target site sequence may refer to sequences derived or engineered from the wild-type LF-RF sequences, including substitutions, insertions, deletions, and truncations to as few as 5 bases. Subsequences of the target site base-pair with subsequences of the bridgeRNA found within the target binding loop. The target site may also be referred to as the “acceptor target,” “acceptor target site,” “target sequence,” or “target site.” In conjunction with these teachings, the target site sequence may comprise LT and RT sequences that are complementary, at least in part, to LTG and RTG sequences of a bridgeRNA molecule, respectively. In some embodiments, in the context of formation of an IS110 transpososome complex, the target site sequence refers to a DNA sequence to which a bridgeRNA sequence is designed to have portions complementarity to both strands of the target site sequence, where base-pairing between a target site sequence and a bridgeRNA sequence promotes the formation of a IS110 transposition complex.

In some embodiments, the 3′ end of the bridgeRNA can further comprise a nucleotide sequence that corresponds to a nucleotide sequence derived from the coding sequence of the recombinase found in the natural IS element. In some embodiments, the 3′ end of the bridgeRNA can further comprise a nucleotide sequence that corresponds to about 100 nucleotides derived from the 5′ end of coding sequence of the recombinase found in the natural IS element. See FIGS. 40A-D. In some embodiments, the 3′ end of the bridgeRNA can further comprise a nucleotide sequence that corresponds to about 100 nucleotides derived from the 5′ end of coding sequence of the recombinase found in the natural IS element (e.g., when bridgeRNA is encoded on the bottom strand of the IS element). In some embodiments, the 5′ end of the bridgeRNA can further comprise a nucleotide sequence that corresponds to a nucleotide sequence derived from the coding sequence of the recombinase found in the natural IS element. In some embodiments, the 5′ end of the bridgeRNA can further comprise a nucleotide sequence that corresponds to about 100 nucleotides derived from the 3′ end of coding sequence of the recombinase found in the natural IS element.

### F. Split Systems

In some embodiments, the present invention contemplates a split system such as described in Berrios K N, Evitt N H, DeWeerd R A, Ren D, Luo M, Barka A, Wang T, Bartman C R, Lan Y, Green A M, Shi J, Kohli R M, Controllable genome editing with split-engineered base editors, Nat Chem Biol., 2021 December; 17(12):1262-1270). In some embodiments, a split IS110 system refers to the expression of subsequences of any IS110 transposase protein from different promoters. It may also refer to a system in which protein sequences derived from IS110 transposase proteins are encoded or delivered as more than one molecule, with the intention that they reconstitute and have functional activity. An example is a first polypeptide encoding a protein comprising the RuvC-like DEDD catalytic domain and a linker and a second polypeptide encoding a protein comprising the transposase domain. In some embodiments, the linker is a coiled-coil domain. Another example is a first polypeptide encoding a protein comprising the RuvC-like DEDD catalytic domain and a second polypeptide encoding a protein comprising a linker and a transposase domain. In some embodiments, the linker is a coiled-coil domain. In some embodiments, the RuvC-like DEDD catalytic domain comprises an amino acid sequence that is 50-100% identical to the RuvC-like DEDD catalytic domain sequences provided in FIG. 16 (SEQ ID NOS: 10176-10523) or SEQ ID NOS: 10524-20350 or 40357-516430 as described in Section C.1, forms a similar tertiary structure to the RuvC-like DEDD catalytic domain as described in Section C.1, comprises an amino acid sequence that is 20-100% identical to the RuvC-like DEDD catalytic domain sequences provided in FIG. 16 (SEQ ID NOS: 10176-10523) or SEQ ID NOS: 10524-20350 or 40357-516430 and forms a similar tertiary structure to the RuvC-like DEDD catalytic domain as described in Section C.1, and/or comprises an amino acid sequence that is 50-100% identical to a protein or protein domain comprising any of the motifs or sequences in FIGS. 21-28 as described in Section C.1. In some embodiments, the linker domain comprises an amino acid sequence that is 50-100% identical to the linker domain sequences provided in FIG. 16 (SEQ ID NOS: 10176-10523) or SEQ ID NOS: 10524-20350 or 40357-516430 as described in Section C.3. In some embodiments, the transposase domain comprises an amino acid sequence that is 50-100% identical to the transposase domain sequences provided in FIG. 16 (SEQ ID NOS: 10176-10523) or SEQ ID NOS: 10524-20350 or 40357-516430 as described in Section C.2, forms a similar tertiary structure to the transposase domain as described in Section C.2, comprises an amino acid sequence that is 20-100% identical to the transposase domain sequences provided in FIG. 16 (SEQ ID NOS: 10176-10523) or SEQ ID NOS: 10524-20350 or 40357-516430 and forms a similar tertiary structure to the transposase domain as described in Section C.2, and/or comprises an amino acid sequence that is 50-100% identical to a protein or protein domain comprising any of the motifs or sequences in FIGS. 29-34 as described in Section C.2.

A split IS110 system may reconstitute the IS110 transposase on its own by binding the bridgeRNA. A split IS110 system may be induced to reconstitute by tethering a known dimerization system, such as FKB-FKBP to each of the two polypeptides similar to the base editing approach cited in Berrios et al., 2021. The IS110 transposase protein may also be delivered in one or more pieces without tethering to an association domain, doing so in such a way that allows reconstitution of the full functional protein, similar to the approach employed for making split-GFP molecules.

Provision of a “pulse” of genome edits may be performed by providing an inducer for the expression of a polypeptide comprising an IS110 transposase or one or more domains of an IS110 transposase or providing a molecule to allow reconstitution of the functional complex. bridgeRNAs may also be split into one or molecules and similarly reconstitute within a transpososome complex.

In one embodiment, the present invention contemplates an approach to engineer IS110 systems by the provision of IS110 domains as individually expressed proteins. One or more individual proteins may comprise a RuvC-like DEDD catalytic domain and linker domain, a linker domain and transposase domain, a RuvC-like DEDD catalytic domain, a linker domain, or a transposase domain. These proteins may be co-expressed, including in the presence of a bridgeRNA, a DNA molecule comprising a donor site sequence, and/or a DNA molecule comprising a target site sequence, for the purposes of genome engineering of any other techniques described herein.

The present invention also contemplates, for example, splitting the bridgeRNA anywhere along its sequence. Such an embodiment is applicable to any bridgeRNA disclosed herein, including those described in Sections D and E. In one embodiment, the bridgeRNA is split so that the target binding loop and donor binding loop are on separate RNA molecules. In one embodiment, a first RNA molecule comprises a stem-loop comprising a target binding loop and a second RNA molecule comprises a stem-loop comprising a donor binding loop. In some embodiments, the first and/or second RNA molecule further comprise portions of a bridgeRNA molecule (e.g., 5′ stem-loop or 3′ extension). In one embodiment, a first RNA molecule comprises a bridgeRNA and a second RNA molecule comprises a stem-loop comprising a target binding loop, optionally wherein one or more of the loops of the bridgeRNA encodes non-targeting guides. In one embodiment, a first RNA molecule comprises a bridgeRNA and a second RNA molecule comprises a stem-loop comprising a donor binding loop, optionally wherein one or more of the loops of the bridgeRNA encodes non-targeting guides. In some embodiments, the portions of the split bridgeRNA described herein are encoded on different DNA molecules. In some embodiments, the portions of the split bridgeRNA described herein are encoded on the same DNA molecule expressed from different promoters. Thus, in some embodiments the system comprises a nucleic acid encoding a first portion of a split bridgeRNA operably linked to a first promoter and a second portion of a split bridgeRNA operably linked to a second promoter.

In certain aspects, provided herein at split bridgeRNA systems that use ribozymes on the 5′ or 3′ end of a bridgeRNA or on any portion of a split bridgeRNA such that cleavage occurs at an intended location (e.g., but not limited to hammerhead (HH) ribozyme, hepatitis delta virus (HDV) ribozyme, or others known in the art.). For example, a ribozyme can be used such that a bridgeRNA can be split after transcription from a single promoter. In one embodiment, an RNA molecule comprising a target binding loop-a HH ribozyme site-a HDV ribozyme site-a donor binding loop results in a target binding loop and donor binding loop as separate RNA molecules after cleavage with ribozyme. As such, in one embodiment, the system comprises a RNA molecule comprising a target binding loop-a first ribozyme site-a second ribozyme site and a donor binding loop. In another embodiment, the system comprises a donor binding loop-a first ribozyme site-a second ribozyme site and a target binding loop. In some embodiments the first and second ribozyme sites are a HH ribozyme site and a HDV ribozyme site. In some embodiments, the system comprises a nucleic acid encoding any of the split bridgeRNAs disclosed herein.

In certain aspects, provided herein at split bridgeRNA systems that uses a group 1 intron between the target binding loop and donor binding loop, such that full-length intron circularization of the group 1 intron results in a target binding loop and donor binding loop as separate RNA molecules. As such, in one embodiment, the system comprises a RNA molecule comprising a target binding loop-a group 1 intron-a donor binding loop. In some embodiments, the system comprises a nucleic acid encoding a RNA molecule comprising a target binding loop-a group 1 intro-a donor binding loop.

Such systems can be used to control the transposition reaction, including for example, providing temporal control. A split bridgeRNA is also contemplated for use in massively parallel combinatorial screens employing pool-on-pool assays of target and donor loops with desired specificities, structures, or functions. All approaches using a split transposase protein or bridgeRNA would be compatible with any embodiment of donor site and target site sequences.

### G. Vectors and Cell Lines

Several aspects of the invention relate to vector systems comprising one or more vectors, or vectors as such comprising nucleic acid sequences encoding the IS110 elements described herein, IS110 transposases described herein, bridgeRNAs described herein, donor site sequences described herein, and/or target site sequences described herein. Vectors can be designed for expression of transcripts (e.g., nucleic acid transcripts, proteins, or enzymes) in prokaryotic or eukaryotic cells. For example, transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of nucleic acid constructs or one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of a recombinant protein (in this case an IS110 transposase). Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

As used herein, minicircle refers to small circular plasmids or DNA vectors that are episomal and are produced as a circular expression cassette devoid of any bacterial plasmid backbone. They can be generated from a parental bacterial plasmid that contains a heterologous nucleic acid and two recombinase target sites by intramolecular (cis-) recombination using a site-specific recombinase, such as PhiC31 integrase. Recombination between the two sites generates a minicircle and a leftover miniplasmid. The minicircle can be recovered via separation from the miniplasmid.

Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

In certain aspects, described herein is a cell comprising a nucleic acid encoding any of the IS110 transposases disclosed herein. In some embodiments, the cell further comprises a nucleic acid encoding a bridgeRNA. In some embodiments, the genome of the cell comprises a donor site sequence or a target site sequence for the IS110 transposase and corresponding bridgeRNA. Such a cell line can be used in a method wherein a nucleic acid comprising a donor site sequence or a target site sequence and a nucleic acid for insertion is introduced into the cell to generate an engineered cell line comprising the nucleic acid of interest inserted into the target site sequence or donor site sequence, respectively. In some embodiments, described herein is a kit comprising a cell comprising a nucleic acid encoding any of the IS110 transposases disclosed herein. In some embodiments, the cell further comprises a nucleic acid encoding a bridgeRNA In some embodiments, the genome of the cell of the kit comprises a donor site sequence or a target site sequence for the IS110 transposases and corresponding bridgeRNA. In some embodiments, the kit further comprises a nucleic acid vector (e.g. plasmid) encoding a bridgeRNA sequence. In some embodiments, in the case that the genome of the cell of the kit comprises a donor site sequence the kit further comprises a nucleic acid vector (e.g. plasmid) comprising a target site sequence. In some embodiments, in the case that the genome of the cell of the kit comprises a target site sequence the kit further comprises a nucleic acid vector (e.g. plasmid) comprising a donor site sequence. In some embodiments, the nucleic acid vector (e.g. plasmid) of the kit further comprises a multicloning site flanked by LE-core (where used) and core (where used)-RE for insertion of a nucleic acid of interest.

In some cases, expression of the components of the IS110 transposase system described herein are under the control of an inducible promoter or repressor element. The inducible promoter or repressor element can be inserted into the promoter region of a nucleic acid sequence encoding one or more components of the IS110 transposase system described herein to provide temporal and/or spatial control of the expression or activity. In some embodiments, a cell can be engineered with a feedback mechanism so that expression of the IS110 transposase is inactivated after a recombination reaction between a donor site sequence and target site sequence has occurred. For example, upon expression of system components, one or more bridgeRNAs may be provided such that the system effectuates recombination at a desired locus in addition to a DNA molecule encoding the IS110 transposase or bridgeRNA such that the DNA sequence is inverted, excised, or inserted into such that the transposase or bridgeRNA is functionally isolated or separated from the promoter driving its expression.

Upon delivery of a nucleic acid encoding an IS110 transposase described herein to a cell, the nucleic acid can be transcribed and translated into a IS110 transposase protein. Upon delivery of a DNA nucleic acid encoding a bridgeRNA described herein to a cell, the DNA nucleic acid can be transcribed into a RNA nucleic acid comprising the bridgeRNA. The IS110 transposase protein can form a complex with the bridgeRNA inside the cell.

### H. Methods of the Invention

In some embodiments, the IS110 transposases described herein are used for genetic engineering and integration of a nucleic acid molecule of interest via site-specific recombination.

In some embodiments, a polynucleotide comprising a cargo and donor site sequence can undergo an insertion reaction with a target site sequence using a IS110 transposase and a bridgeRNA which is specific for the donor site sequence and target site sequence. In some embodiments, a polynucleotide comprising a cargo and target site sequence can undergo an insertion reaction with a donor site sequence using a IS110 transposase and a bridgeRNA which is specific for the donor site sequence and target site sequence. In some embodiments, a polynucleotide sequence located between a donor site sequence and a target site sequence can be excised or inverted using a IS110 transposase and a bridgeRNA which is specific for the donor site sequence and target site sequence. In some embodiments, a polynucleotide sequence comprising a cargo and two different donor sequences can undergo an insertion reaction with two different target site sequences using a IS110 transposase and two different bridgeRNAs which are specific for each of the donor site sequences and target site sequences. In some embodiments, a polynucleotide sequence comprising a cargo and two different target sequences can undergo an insertion reaction with two different donor site sequences using a IS110 transposase and two different bridgeRNAs which are specific for each of the donor site sequences and target site sequences. In some embodiments, a polynucleotide sequence comprising a cargo and a donor site sequence and a target site sequence can undergo an insertion reaction with a target site sequence and a donor site sequence respectively, using a IS110 transposase and two different bridgeRNAs which are specific for each of the donor site sequences and target site sequences.

In certain aspects, disclosed herein is a method of integrating a DNA molecule of interest into a sequence specific site of a DNA of interest of a cell, the method comprising introducing into the cell: a nucleic acid editing system described herein. In some embodiments the cell is a prokaryotic cell. In some embodiments, the cell is a bacterial cell. In some embodiments, the cell is a eukaryotic cell. In some embodiments the cell is an archeal cell. In some embodiments, the cell is a fungus cell. In some embodiments, the cell is a yeast cell. In some embodiments, the cell is a plant cell. In some embodiments, the cell is a mammalian cell. In some embodiments, the cell is a human cell. In some embodiments, the DNA of interest of the cell comprises a donor site sequence and the DNA molecule of interest is part of a nucleic acid of the nucleic acid editing system comprising a target site sequence. In some embodiments, the DNA of interest of the cell comprises a target site sequence and the DNA molecule of interest is part of a nucleic acid of the nucleic acid editing system comprising a donor site sequence. In some embodiments, the DNA molecule of interest is flanked by two different donor site sequences. In some embodiments, the DNA molecule of interest is flanked by two different target site sequences. In some embodiments, the DNA molecule of interest is flanked by a donor site sequence and a target site sequence that corresponds to a different donor site sequence. In some embodiments, the sequence of the bridgeRNA was engineered before introduction of the nucleic acid editing system to bind to the donor site sequence and target site sequence. In some embodiments, the DNA of interest of the cell is the genome of the cell. In some embodiments, the DNA of interest of the cell is a plasmid. In some embodiments, the method comprises performing cassette exchange (e.g., recombinase mediated cassette exchange (RMCE) as described in Section E1. In some embodiments, the method comprises inserting one or more minicircles comprising a DNA molecule of interest as described in Section E1.

In certain aspects, disclosed herein is a method of inverting a DNA sequence of a DNA of interest of a cell, the method comprising introducing into the cell: a nucleic acid editing system disclosed herein, wherein a target site sequence and a donor site sequence are present on the same DNA molecule of interest and the LD of the donor site sequence and the RT of the target site sequence are on the same DNA strand and RD of the donor site sequence and LT of the target site sequence are on the same DNA strand. In some embodiments, the target DNA of interest of the cell is the genome of the cell. In some embodiments, the sequence of the bridgeRNA was engineered before introduction of the nucleic acid editing system to bind to the donor site sequence and the target site sequence.

In certain aspects, disclosed herein is a method of excising a DNA sequence of a DNA of interest of a cell, the method comprising introducing into the cell: a nucleic acid editing system disclosed herein, wherein a target site sequence and a donor site sequence are present on the same DNA molecule of interest and the LD of the donor site sequence and the LT of the target site sequence are on the same DNA strand. In some embodiments, the target DNA of interest of the cell is the genome of the cell. In some embodiments, the sequence of the bridgeRNA was engineered before introduction of the nucleic acid editing system to bind to the donor site sequence and the target site sequence.

In certain aspects, disclosed herein is a method of translocating DNA sequences between two linear DNA molecules of interest, the method comprising introducing into a cell: a nucleic acid editing system disclosed herein, wherein a donor site sequence is present on a first linear DNA molecule and a target site sequence is present on a second linear DNA molecule. In some embodiments, the linear DNA molecules of interest of the cell are chromosomes of the cell. In some embodiments, the sequence of the bridgeRNA was engineered before introduction of the nucleic acid editing system to bind to the donor site sequence and target site sequence.

In certain aspects, disclosed herein is a method of screening bridgeRNA for compatibility with a given donor site sequence and target site sequence. In some embodiments multiple pairs of donor site sequence and target site sequence are screened for ability to effectuate a transposition reaction and/or specificity and/or efficiency of the reaction. In some embodiments, one or more of LTG, RTG, LDG, and RDG of a bridgeRNA are modified (e.g., mutated, lengthened, shortened) and screened for ability to effectuate a transposition reaction and/or specificity and/or efficiency of the reaction. In some embodiments, the method comprises introducing into a cell the bridgeRNAs for screening and a donor molecule and measuring recombination, wherein the cell expresses a IS110 transposase. In some embodiments, recombination is measured using a reporter or sequencing based assay.

The invention comprehends the use of the compositions of the current invention to establish and utilize transgenic cells or organisms. The invention provides a non-naturally occurring or engineered composition, or one or more polynucleotides encoding components of said composition, or vector or delivery systems comprising one or more polynucleotides encoding components of said composition for use in modifying a target cell in vivo, ex vivo or in vitro and, may be conducted in a manner that alters the cell such that once modified the progeny or cell line of the modified cell retains the altered phenotype. The modified cells and progeny may be part of a multi-cellular organism such as a plant or animal with ex vivo or in vivo application of the IS110 system to desired cell types.

The invention may be a therapeutic method of treatment. The therapeutic method of treatment may comprise gene or genome editing, or gene therapy. A related method of the invention may be used to create an organism or cell that may be used to model and/or study genetic or epigenetic conditions of interest, such as through a model of mutations of interest or as a disease model. As used herein, “disease” refers to a disease, disorder, or indication in a subject. For example, a method of the invention may be used to create an organism or cell that comprises a modification in one or more nucleic acid sequences associated with a disease, or an organism or cell in which the expression of one or more nucleic acid sequences associated with a disease are altered. Such a nucleic acid sequence may encode a disease associated protein sequence or may be a disease associated control sequence. Accordingly, it is understood that in embodiments of the invention, a subject, patient, organism or cell can be a non-human subject, patient, organism or cell. Thus, the invention provides an organism or cell, produced by the present methods, or a progeny thereof. The progeny may be a clone of the produced organism, or may result from sexual reproduction by crossing with other individuals of the same species to introgress further desirable traits into their offspring. The cell may be in vivo or ex vivo in the cases of multicellular organisms. In the instance where the cell is in culture, a cell line may be established if appropriate culturing conditions are met and preferably if the cell is suitably adapted for this purpose (for instance a stem cell). Bacterial cell lines produced by the invention are also envisaged. Hence, cell lines are also envisaged.

In some methods, the disease model can be used to study the effects of mutations on the organism or cell and development and/or progression of the disease using measures commonly used in the study of the disease. Alternatively, such a disease model is useful for studying the effect of a pharmaceutically active compound on the disease.

In some methods, the disease model can be used to assess the efficacy of a potential gene therapy strategy. That is, a disease-associated gene or polynucleotide can be modified such that the disease development and/or progression is inhibited or reduced. In particular, the method comprises modifying a disease-associated gene or polynucleotide such that an altered protein is produced and, as a result, the animal or cell has an altered response. Accordingly, in some methods, a genetically modified organism may be compared with an organism predisposed to development of the disease such that the effect of the gene therapy event may be assessed.

In another embodiment, this invention provides a method of developing a biologically active agent that modulates a cell signaling event associated with a disease gene. The method comprises contacting a test compound with a cell comprising one or more vectors that drive expression of the IS110 system of the present invention; and detecting a change in a readout that is indicative of a reduction or an augmentation of a cell signaling event associated with, e.g., a mutation in a disease gene contained in the cell.

A cell model or animal model can be constructed in combination with the method of the invention for screening a cellular function change. Such a model may be used to study the effects of a cellular DNA sequence modified by the IS110 system of the invention on a cellular function of interest. For example, a cellular function model may be used to study the effect of a modified cellular DNA sequence on intracellular signaling or extracellular signaling. Alternatively, a cellular function model may be used to study the effects of a modified cellular DNA sequence on sensory perception. In some such models, one or more cellular DNA sequences associated with a signaling biochemical pathway in the model are modified.

A transgenic cell in which one or more nucleic acids encoding one or more of the components of the present invention are provided or introduced operably connected in the cell with a regulatory element comprising a promoter of one or more genes of interest. As used herein, the term “IS110 transgenic cell” refers to a cell, such as a eukaryotic cell, in which an IS110 element or components thereof (IS110 transposase, donor site sequence, bridgeRNA, target site sequence for the IS110 element, or any combination thereof) has been genomically integrated. The nature, type, or origin of the cell are not particularly limiting according to the present invention. Also, the way in which the IS110 transgene is introduced in the cell may vary and can be any method as is known in the art. In certain embodiments, the IS110 transgenic cell is obtained by introducing the IS110 transgene in an isolated cell. In certain other embodiments, the IS110 transgenic cell is obtained by isolating cells from a IS110 transgenic organism. By means of example, and without limitation, the IS110 transgenic cell as referred to herein may be derived from a IS110 transgenic eukaryote, such as a IS110 knock-in eukaryote. Reference is made to WO 2014/093622 (PCT/US 13/74667), incorporated herein by reference. Methods of U.S. Pat. Nos. 8,771,985 and 9,567,573 assigned to Sangamo Biosciences, Inc. directed to targeting the Rosa locus may be modified to utilize the IS110 system of the present invention. Methods of US Patent Publication No. 20130236946 assigned to Cellectis directed to targeting the Rosa locus may also be modified to utilize the IS110 system of the present invention. By means of further example reference is made to Platt et. al. (Cell; 159(2):440-455 (2014)), describing a Cas9 knock-in mouse, which is incorporated herein by reference. The IS110 transgene can further comprise a Lox-Stop-polyA-Lox(LSL) cassette thereby rendering IS110 expression inducible by Cre recombinase. Alternatively, the IS110 transgenic cell may be obtained by introducing the IS110 transgene in an isolated cell. Delivery systems for transgenes are well known in the art. By means of example, the IS110 system may be delivered in for instance a eukaryotic cell by means of a vector (e.g., AAV, adenovirus, lentivirus) and/or particle and/or nanoparticle delivery, as also described herein elsewhere.

In one embodiment, the present invention contemplates a method for controlling gene expression via utilizing an IS110 transposase, bridgeRNA, donor site sequence, target site sequence or any combination thereof. The IS110 transposase may be expressed as a fusion protein with known epigenetic modifiers, including activator, repressor, or other DNA modifying domains such as but not limited to VPR, VP64, p65, PRDM9, LSD1, SMYD3, BAF, HP1, G9A, KRAB, EZH2, FOX1, DOTIL, p300, HDAC3, DNMT3A, M.SSI, TET1, DNMT3L. The IS110 transposase may also be expressed as a fusion protein encoding recruitment domains including but not limited to Suntag, FKBP/FRB and derivatives, CRY2/CIB1, SpyTag, SnoopTag/SnoopCatcher for recruitment of partner-domain fused epigenetic modifiers. The bridgeRNA may also be modified to encode RNA aptamers, including but not limited to, MS2, PP7, com, PUF, for the recruitment of epigenetic modifiers fused to the appropriate aptamer binding domain. In any of these described embodiments, IS110 transposases, including the RuvC-like DEDD domain, a linker domain, and Tnp domains, may be modified to be catalytically inactive. See Nakamura et al. 2021; “The CRISPR/Cas System: Emerging Technology and Application” n.d.; Tak et al. 2017; Zhai et al. 2022; Lebar et al. 2020.

In one embodiment, the present invention contemplates the use of IS110 transposases as DNA targeting domains for cellular DNA engineering tools. The IS110 transposase may be catalytically inactivated such that it does not mediate recombination and is fused to cellular DNA engineering protein domains. The IS110 transposase may still associate with its bridgeRNA and utilize it to bind to DNA sites of interest and position the fusion protein at or close to the DNA site(s) of interest that may be edited by the fused DNA engineering protein domains. In some embodiments, the non-transposase polypeptide domain of such a fusion provides an additional function, such as, but not limited to, nucleic acid modification, transcriptional activation, transcriptional repression, and epigenetic modification. IS110 base editors are contemplated as fusions of IS110 transposases to domains including, but not limited to, rAPOBEC1, hAID, pmCDA1 UGI, TadA, ADAR, UNG. IS110 prime editors are contemplated as fusions of IS110 transposases to domains including, but not limited to, MMLV RT, Marathon RT, GsI-IIC. In an embodiment of an IS110 prime editor, the bridgeRNA would be engineered to include a primer binding site and RT template containing a desired nucleic acid modification. In another embodiment, the present invention contemplates the use of IS110s solely as targeting domains for large cargo insertion systems. The IS110 transposases may be fused to integrases, including, but not limited to, Bxb1, PhiC31, Pa01, Kp03, Dn29, Si74. IS110 transposases fused to integrases may also be fused to protein domains including, but not limited to, those found in prime editors, such as MMLV RT. In an embodiment of an IS110 integrase prime editor, a bridgeRNA would be engineered to include a primer binding site and RT template with edit. See Gaudelli et al. 2017; Komor et al. 2016; Anzalone et al. 2019; Grunewald et al. 2022; Durrant et al. 2022; Anzalone et al. 2021. IS110 transposases may also be tethered to domains which can recruit proteins tethered to binding partners, such as an IS110 tethered to a FKBP domain and a DNA engineering domain tethered to a FBP domain.

Embodiments of the invention also comprehend use of nucleic acid constructs, fusion proteins, vectors, amplicons, expression vectors, cells, eukaryotic cells, mammalian cells, expression plasmids, mRNAs, viral vectors, adenovirus vectors, lentivirus vectors, or adeno-associated virus vectors, or methods for altering, modifying, or modulating transcription in a cell, or for integrating a desired nucleotide sequence into DNA or cellular DNA in such a way obvious by those skilled in the art according to previously demonstrated approaches for engineering and repurposing similar systems to the invention.

For example, in prime editing, such as described in WO2020191153, U.S. Pat. No. 11,447,770, or US20220054239 one would use the IS110 system of the invention combined with a polymerase (e.g. a reverse transcriptase) for template gene edits instead of a Cas9 system. For another example, in twin prime editing, such as described in WO2021226558, one would use the IS110 system of the invention instead of the first, second, third, fourth, etc. prime editor complexes. In another example, in PASTE, such as described in Ioannidi et al., 2021, US20220154224, or Tou et al., 2022, one would use the IS110 system of the invention instead of the Cas9-based prime editing system. In another example, in Gene Writer™ gene editor systems, such as described in WO2020047124, one would use the IS110 system instead of the retrotransposon-derived DNA binding domain of the Gene Writer™ gene editor system. In another example, in base editing, such as described in Anzalone et al., 2020 or Chen et al., 2021, one would use the IS110 system of the invention instead of the base editor system. In examples of editing systems with a targeting domain and integrase domain, such as PASTE, TwinPrime, or Gene Writer, an IS110 system can be used as an integrase paired with the Cas9 or retrotransposon-derived DNA binding domain.

In one embodiment, the present invention contemplates the use of IS110s as RNA targeting and RNA modifying systems. C/D box RNAs and other snoRNAs are involved in ribosomal RNA processing and RNA methylation in archaea and eukaryotes. C/D box RNAs have structural homology to the bridgeRNA structure. C/D box RNA binding proteins have homology to IS110 transposases, such as NOP58, NOP56, and SNU13 (15.5K) in humans, Nop58p, Nop56p, and Snu13p in yeast, and Nop5 and L7Ae in archaea. IS110 transposases may be engineered to harbor C/D box motif binding domains from these homologous systems, enabling complexation of IS110 transposases with naturally occurring C/D box RNAs. IS110 transposases may also be engineered to include a fibrillarin binding domain for the purposes of RNA methylation. An IS110 transposase modified to bind fibrillarin may be used in combination with a bridgeRNA to target endogenous RNA transcripts for methylation, similar to the function of NOP58/NOP56/SNU13p/Fibrillarin C/D box RNA complexes in human cells. In another embodiment, IS110 transposases may be fused to fibrillarin or other RNA methylation domains for the targeted methylation of endogenous transcripts. IS110 systems used for RNA methylation encode specificity for RNAs within the donor binding or target binding loops of the bridgeRNA. IS110 transposase mediated RNA binding may also be used for RNA knockdown, similar to approaches used with anti-sense oligonucleotides (ASOs), RNA interference, and Cas13-mediated RNA targeting and cleavage.

In one embodiment, the present invention contemplates a method for performing genetic screens utilizing IS110 systems. Screens may be performed by massively parallel programmable genomic or episomal insertions, excisions or inversions. A sequence including, but not limited to, an encoded epitope tag, stop codon, fluorescent protein, or some other protein of interest may be inserted into the genome, advantageously within a protein coding sequence, for the determination of phenotypic change as a result of the insertion. Insertions, excisions and inversions may be made in a targeted fashion for the study of individual chromosomes, loci, or genes or in such a way as to study a set of chromosomes, loci, genes and the proteins they encode. Insertions, excisions and inversions may be done in such a way to generate pooled, targeted knockouts to study monogenic and polygenic phenotypes, including those relevant to human disease.

The present invention contemplates a method for engineering genomes for the study of genome topology, epigenomics, gene regulation, and for the treatment of disease. In one embodiment, polynucleotide sequences, advantageously sequences encoding one or more donor site sequences or target site sequences, may be inserted with IS110 transposases into gene clusters, gene regulatory regions, topologically associated domains, chromosomes, and other genomic regions in such a fashion as to disrupt, modify, or replicate these sequences in any location in the genome. In another embodiment, polynucleotide sequences, both naturally existing and engineered, may be recognized by IS110 transposases in such a way that sequences within gene clusters, gene regulatory regions, topologically associated domains (TADs), chromosomes, and other genomic regions are precisely excised from the genome or inverted. In another embodiment, polynucleotide sequences, both naturally existing and engineered, may be recognized by IS110 transposases in such a way that sequences within gene clusters, gene regulatory regions, topologically associated domains, chromosomes, and other genomic regions are precisely integrated into another sequence, producing rearrangements of aforementioned sequences. Advantageously, such an approach may be used to produce and study chromosomal translocations, especially in the context of diseases involving chromosomal translocations such as many leukemias, Ewing's sarcoma, Down's syndrome, and other diseases. In another embodiment, aforementioned approaches may be advantageously performed in a massively parallel fashion using the programmable bridgeRNA to screen for phenotypic effects relating to rearrangement, insertion, and deletion of sequences from the genome.

In some embodiments, the IS110 system may be utilized for large excisions or deletions or chromosomal translocations. The IS110 system may be utilized to integrate enhancer sequences into new genomic contexts. The IS110 system may be utilized to integrate or destroy a binding site for proteins that dictate genome structure, such as CTCF. The IS110 system may be utilized in chromosomal fusions, such as those often found in cancers and heritable diseases.

### I. Delivery Methods

In some embodiments, methods for introducing an IS110 transposase-bridgeRNA ribonucleoprotein complex into a cell (e.g., a hematopoietic cell or hematopoietic stem cell, including, e.g., such cells from humans) include forming a reaction mixture containing the protein or ribonucleoprotein complex and introducing transient holes in the extracellular membrane of the cell. Such transient holes can be introduced by a variety of methods, including, but not limited to, electroporation, cell squeezing, or contacting with nanowires or nanotubes. Generally, the transient holes are introduced in the presence of the protein or ribonucleoprotein complex and the protein or ribonucleoprotein complex is allowed to diffuse into the cell.

Methods, compositions, and devices for electroporating cells to introduce a protein or ribonucleoprotein complex can include those described in the examples herein. Additional or alternative methods, compositions, and devices for electroporating cells to introduce a protein or ribonucleoprotein complex can include those described in WO/2006/001614 or Kim, J. A. et al. Biosens. Bioelectron. 23, 1353-1360 (2008). Additional or alternative methods, compositions, and devices for electroporating cells to introduce a protein or ribonucleoprotein complex can include those described in U.S. Patent Appl. Pub. Nos. 2006/0094095; 2005/0064596; or 2006/0087522. Additional or alternative methods, compositions, and devices for electroporating cells to introduce a protein or ribonucleoprotein complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842. Additional or alternative methods, compositions, and devices for electroporating cells to introduce a protein or ribonucleoprotein complex can include those described in Geng, T. et al. J. Control Release 144, 91-100 (2010); and Wang, J., et al. Lab. Chip 10, 2057-2061 (2010).

In some cases, the methods or compositions described in the patents or publications cited herein are modified for protein or ribonucleoprotein delivery. Such modification can include increasing or decreasing voltage, pulse length, or the number of pulses. Such modification can further include modification of buffers, media, electrolytic solutions, or components thereof. Electroporation can be performed using devices known in the art, such as a Bio-Rad Gene Pulser Electroporation device, an Invitrogen Neon transfection system, a MaxCyte transfection system, a Lonza Nucleofection device, a NEPA Gene NEPA21 transfection device, a flow though electroporation system containing a pump and a constant voltage supply, or other electroporation devices or systems known in the art.

Methods, compositions, and devices for squeezing or deforming a cell to introduce a protein or ribonucleoprotein complex can include those described herein. Additional or alternative methods, compositions, and devices can include those described in Nano Lett. 2012 Dec. 12; 12(12):6322-7; Proc Natl Acad Sci USA. 2013 Feb. 5; 110(6):2082-7; J Vis Exp. 2013 Nov. 7; (81):e50980; and Integr Biol (Camb). 2014 April; 6(4):470-5. Additional or alternative methods, compositions, and devices can include those described in U.S. Patent Appl. Publ. No. 2014/0287509. Generally, the protein or ribonucleoprotein complex is provided in a reaction mixture containing the cell and the reaction mixture is forced through a cell deforming orifice or constriction. In some cases, the constriction is smaller than the diameter of the cell. In some cases, the constriction contains cell-deforming components such as regions of strong electrostatic charge, regions of hydrophobicity, or regions containing nanowires or nanotubes. The forcing can introduce transient pores into a cell membrane of the cell allowing the protein or ribonucleoprotein complex to enter the cell through the transient pores. In some cases, squeezing or deforming a cell to introduce the protein or ribonucleoprotein can be effective even when the cell is in a non-dividing state.

Methods for introducing a protein or ribonucleoprotein complex into a cell include forming a reaction mixture containing the protein or ribonucleoprotein complex and contacting the cell with the protein or ribonucleoprotein complex to induce receptor-mediated internalization. Compositions and methods for receptor mediated internalization are described, e.g., in Wu et al., J. Biol. Chem. 262, 4429-4432 (1987); and Wagner et al., Proc. Natl. Acad. Sci. USA 87, 3410-3414 (1990). Generally, the receptor-mediated internalization is mediated by interaction between a cell surface receptor and a ligand fused to the protein or fused to the ribonucleoprotein complex (e.g., covalently attached or fused to an RNA in the ribonucleoprotein complex). The ligand can be any protein, small molecule, polymer, or fragment thereof that binds to, or is recognized by, a receptor on the surface of the cell. An exemplary ligand is an antibody or an antibody fragment (e.g., scFv).

In some embodiments, the reaction mixture for introducing the protein or ribonucleoprotein complex into the cell can contain a nucleic acid for directing binding to the target genomic region.

In some embodiments, delivery is via a nucleic acid (e.g., plasmid(s)) transfected into a cell. The transfected nucleic acids (e.g., plasmid(s)) can comprise an expression vector for an IS110 transposase, a nucleic acid (e.g., plasmid) comprising a donor molecule comprising a donor site sequence for recombination with the cell's genome at a target site sequence or a donor molecule comprising a target site sequence for recombination with the cell's genome at a donor site sequence, and an expression vector for bridgeRNA.

The nucleic acids may be delivered using adeno associated virus (AAV), lentivirus, adenovirus or other viral vector types, or combinations thereof. The nucleic acids can be packaged into one or more viral vectors. The nucleic acids can be packaged into virions using appropriate packaging cells lines as known in the art. In some embodiments, the viral vector is delivered to the tissue of interest by, for example, an intramuscular injection, while other times the viral delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector chosen, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. Such a dosage formulation is readily ascertainable by one skilled in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 2020) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose is at least about 1×10⁶ particles (e.g., about 1×10⁶-1×10¹² particles), at least about 1×10⁷ particles (e.g, about 1×10⁷-1×10⁹ particles or about 1×10⁷-1×10¹² particles), at least about 1×10⁸ particles (e.g., about 1×10⁸-1×10¹¹ particles or about 1×10⁸-1×10¹² particles), and at least about 1×10⁹ particles (e.g., about 1×10⁹-1×10¹⁰ particles or about 1×10⁹-1×10¹² particles), or at least about 1×10¹⁰ particles (e.g., about 1×10¹⁰-1×10¹² particles) of the adenoviral vector. Alternatively, the dose comprises no more than about 1×10¹⁴ particles. Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing about 1×10¹⁰ to about 1×10¹² functional AAV/ml solution. The dosage may be adjusted to balance therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In an embodiment herein the delivery is via a plasmid(s). In such plasmid compositions, the dosage should be a sufficient amount of plasmid to elicit a response. For instance, suitable quantities of plasmid DNA in plasmid compositions can be from about 0.1 to about 2 mg, or from about 1 g to about 10 g.

The doses herein are based on an average 70 kg individual. The frequency of administration is within the ambit of the medical or veterinary practitioner (e.g., physician, veterinarian), or scientist skilled in the art. Mice used in experiments are about 20 g. From that which is administered to a 20 g mouse, one can extrapolate to a 70 kg individual.

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentivirus may be purified as follows. Viral supernatants were harvested after 48 hours. Supernatants were first cleared of debris and filtered through a 0.45 um low protein binding (PVDF) filter. They were then spun in a ultracentrifuge for 2 hours at 24,000 rpm. Viral pellets were resuspended in 50 ul of DMEM overnight at 4 C. They were then aliquotted and immediately frozen at −80 C.

In another embodiment, minimal non-primate lentiviral vectors based on the equine infectious anemia virus (EIAV) are also contemplated, especially for ocular gene therapy (see, e.g., Balagaan, J Gene Med 2006; 8: 275-285, Published online 21 Nov. 2005 in Wiley InterScienc; available at the website: interscience.wiley.com. DOI: 10.1002/jgm.845). In another embodiment, RetinoStat®, an equine infectious anemia virus-based lentiviral gene therapy vector that expresses angiostatic proteins endostain and angiostatin that is delivered via a subretinal injection for the treatment of the web form of age-related macular degeneration is also contemplated (see, e.g., Binley et al., HUMAN GENE THERAPY 23:980-991 (September 2012)) may be modified for the system of the present invention.

In another embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the IS110 transposase system of the present invention. A minimum of 2.5×10⁶ CD34+ cells per kilogram patient weight may be collected and prestimulated for 16 to 20 hours in X-VIVO 15 medium (Lonza) containing 2 micro mol/L-glutamine, stem cell factor (100 ng/ml), Flt-3 ligand (Flt-3L) (100 ng/ml), and thrombopoietin (10 ng/ml) (CellGenix) at a density of 2×10⁶ cells/ml. Prestimulated cells may be transduced with lentiviral at a multiplicity of infection of 5 for 16 to 24 hours in 75-cm² tissue culture flasks coated with fibronectin (25 mg/cm²) (RetroNectin, Takara Bio Inc.).

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, 20110117189; 20090017543; 20070054961, and 20100317109. Lentiviral vectors have also been disclosed for delivery to the brain, see, e.g., US Patent Publication Nos. 20110293571; 20110293571, 20070025970, and 20090111106 and U.S. Pat. No. 7,259,015.

Several types of particle delivery systems and/or formulations are known to be useful in a diverse spectrum of biomedical applications. In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm.

As used herein, a particle delivery system/formulation is defined as any biological delivery system/formulation which includes a particle in accordance with the present invention. A particle in accordance with the present invention is any entity having a greatest dimension (e.g. diameter) of less than 100 microns (m). In some embodiments, inventive particles have a greatest dimension of less than 10 microns. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less.

In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarisation interferometry and nuclear magnetic resonance (NMR). Characterization (dimension measurements) may be made as to native particles (i.e., preloading) or after loading of the cargo (herein cargo refers to one or more nucleic acids and/or vectors encoding the same, and may include additional components, carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS).

Particles delivery systems within the scope of the present invention may be provided in any form, including but not limited to solid, semi-solid, emulsion, or colloidal particles. As such any of the delivery systems described herein, including but not limited to, e.g., lipid-based systems, liposomes, micelles, microvesicles, exosomes, or gene gun may be provided as particle delivery systems within the scope of the present invention.

In terms of this invention, it is preferred to have one or more components of the system delivered using nanoparticles or lipid envelopes. Nucleic acid sequences encoding the IS110 elements described herein, IS110 transposases or nucleic acid sequences encoding IS110 transposases described herein, bridgeRNAs described herein, donor site sequences described herein, and/or target site sequences described herein may be delivered simultaneously using nanoparticles or lipid envelopes. Other delivery systems or vectors may be used in conjunction with the nanoparticle aspects of the invention.

In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm.

Nanoparticles encompassed in the present invention may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention.

Semi-solid and soft nanoparticles have been manufactured, and are within the scope of the present invention. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants.

For example, Su X, Fricke J, Kavanagh D G, Irvine D J (“In vitro and in vivo mRNA delivery using lipid-enveloped pH-responsive polymer nanoparticles” Mol Pharm. 2011 Jun. 6; 8(3):774-87. doi: 10.1021/mp100390w. Epub 2011 Apr. 1) describes biodegradable core-shell structured nanoparticles with a poly(β-amino ester) (PBAE) core enveloped by a phospholipid bilayer shell. These were developed for in vivo mRNA delivery. The pH-responsive PBAE component was chosen to promote endosome disruption, while the lipid surface layer was selected to minimize toxicity of the polycation core.

In one embodiment, nanoparticles based on self assembling bioadhesive polymers are contemplated, which may be applied to oral delivery of peptides, intravenous delivery of peptides and nasal delivery of peptides, all to the brain. Other embodiments, such as oral absorption and ocular delivery of hydrophobic drugs are also contemplated. The molecular envelope technology involves an engineered polymer envelope which is protected and delivered to the site of the disease (see, e.g., Mazza, M. et al. ACSNano, 2013. 7(2): 1016-1026; Siew, A., et al. Mol Pharm, 2012. 9(1):14-28; Lalatsa, A., et al. J Contr Rel, 2012. 161(2):523-36; Lalatsa, A., et al., Mol Pharm, 2012. 9(6):1665-80; Lalatsa, A., et al. Mol Pharm, 2012. 9(6):1764-74; Garrett, N. L., et al. J Biophotonics, 2012. 5(5-6):458-68; Garrett, N. L., et al. J Raman Spect, 2012. 43(5):681-688; Ahmad, S., et al. J Royal Soc Interface 2010. 7:S423-33; Uchegbu, I. F. Expert Opin Drug Deliv, 2006. 3(5):629-40; Qu, X., et al. Biomacromolecules, 2006. 7(12):3452-9 and Uchegbu, I. F., et al. Int J Pharm, 2001. 224:185-199). Doses of about 5 mg/kg are contemplated, with single or multiple doses, depending on the target tissue.

In one embodiment, nanoparticles that can deliver nucleic acids to a cancer cell to stop tumor may be used/and or adapted to the IS110 transposase system of the present invention. In particular, fully automated, combinatorial systems for the synthesis, purification, characterization, and formulation of new biomaterials and nanoformulations. See, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93.

U.S. Pat. No. 8,969,353 relates to lipidoid compounds that are also particularly useful in the administration of polynucleotides, which may be applied to deliver the IS110 transposase system of the present invention. In one aspect, the aminoalcohol lipidoid compounds are combined with an agent to be delivered to a cell or a subject to form microparticles, nanoparticles, liposomes, or micelles. The agent to be delivered by the particles, liposomes, or micelles may be in the form of a gas, liquid, or solid, and the agent may be a polynucleotide, protein, peptide, or small molecule. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

U.S. Pat. No. 8,969,353 also provides methods of preparing the aminoalcohol lipidoid compounds. One or more equivalents of an amine are allowed to react with one or more equivalents of an epoxide-terminated compound under suitable conditions to form an aminoalcohol lipidoid compound of the present invention. In certain embodiments, all the amino groups of the amine are fully reacted with the epoxide-terminated compound to form tertiary amines. In other embodiments, all the amino groups of the amine are not fully reacted with the epoxide-terminated compound to form tertiary amines thereby resulting in primary or secondary amines in the aminoalcohol lipidoid compound. These primary or secondary amines are left as is or may be reacted with another electrophile such as a different epoxide-terminated compound. As will be appreciated by one skilled in the art, reacting an amine with less than excess of epoxide-terminated compound will result in a plurality of different aminoalcohol lipidoid compounds with various numbers of tails. Certain amines may be fully functionalized with two epoxide-derived compound tails while other molecules will not be completely functionalized with epoxide-derived compound tails. For example, a diamine or polyamine may include one, two, three, or four epoxide-derived compound tails off the various amino moieties of the molecule resulting in primary, secondary, and tertiary amines. In certain embodiments, all the amino groups are not fully functionalized. In certain embodiments, two of the same types of epoxide-terminated compounds are used. In other embodiments, two or more different epoxide-terminated compounds are used. The synthesis of the aminoalcohol lipidoid compounds is performed with or without solvent, and the synthesis may be performed at higher temperatures ranging from 30-100 C. The prepared aminoalcohol lipidoid compounds may be optionally purified. For example, the mixture of aminoalcohol lipidoid compounds may be purified to yield an aminoalcohol lipidoid compound with a particular number of epoxide-derived compound tails. Or the mixture may be purified to yield a particular stereo- or regioisomer. The aminoalcohol lipidoid compounds may also be alkylated using an alkyl halide (e.g., methyl iodide) or other alkylating agent, and/or they may be acylated.

U.S. Pat. No. 8,969,353 also provides libraries of aminoalcohol lipidoid compounds prepared by the inventive methods. These aminoalcohol lipidoid compounds may be prepared and/or screened using high-throughput techniques involving liquid handlers, robots, microtiter plates, computers, etc. In certain embodiments, the aminoalcohol lipidoid compounds are screened for their ability to transfect polynucleotides or other agents (e.g., proteins, peptides, small molecules) into the cell.

U.S. Pat. No. 9,193,827 relates to a class of poly(beta-amino alcohols) (PBAAs) has been prepared using combinatorial polymerization. The inventive PBAAs may be used in biotechnology and biomedical applications as coatings (such as coatings of films or multilayer films for medical devices or implants), additives, materials, excipients, non-biofouling agents, micropatterning agents, and cellular encapsulation agents. When used as surface coatings, these PBAAs elicited different levels of inflammation, both in vitro and in vivo, depending on their chemical structures. The large chemical diversity of this class of materials allowed the identification of polymer coatings that inhibit macrophage activation in vitro. Furthermore, these coatings reduce the recruitment of inflammatory cells, and reduce fibrosis, following the subcutaneous implantation of carboxylated polystyrene microparticles. These polymers may be used to form polyelectrolyte complex capsules for cell encapsulation. The invention may also have many other biological applications such as antimicrobial coatings, DNA or siRNA delivery, and stem cell tissue engineering. The teachings of U.S. Pat. No. 9,193,827 may be applied to the system of the present invention.

In another embodiment, lipid nanoparticles (LNPs) are contemplated. Doses of about 0.01 to about 1 mg per kg of body weight administered intravenously are contemplated. Medications to reduce the risk of infusion-related reactions are contemplated, such as dexamethasone, acetampinophen, diphenhydramine or cetirizine, and ranitidine are contemplated. Multiple doses of about 0.3 mg per kilogram every 4 weeks for five doses are also contemplated. Lipids include, but are not limited to, DLin-KC2-DMA4, C12-200 and colipids disteroylphosphatidyl choline, cholesterol, and PEG-DMG may be formulated RNA instead of siRNA (see, e.g., Novobrantseva, Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). The final lipid:siRNA weight ratio may be ˜12:1 and 9:1 in the case of DLin-KC2-DMA and C12-200 lipid nanoparticles (LNPs), respectively. The formulations may have mean particle diameters of ˜80 nm with >90% entrapment efficiency. A 3 mg/kg dose may be contemplated.

LNPs have been shown to be highly effective in delivering siRNAs to the liver (see, e.g., Tabernero et al., Cancer Discovery, April 2013, Vol. 3, No. 4, pages 363-470) and are therefore contemplated for delivering components of the IS110 transposases system to the liver, such as the bridgeRNA. A dosage of about four doses of 6 mg/kg of the LNP (or bridgeRNA) every two weeks may be contemplated. Tabernero et al. demonstrated that tumor regression was observed after the first 2 cycles of LNPs dosed at 0.7 mg/kg, and by the end of 6 cycles the patient had achieved a partial response with complete regression of the lymph node metastasis and substantial shrinkage of the liver tumors. A complete response was obtained after 40 doses in this patient, who has remained in remission and completed treatment after receiving doses over 26 months. Two patients with RCC and extrahepatic sites of disease including kidney, lung, and lymph nodes that were progressing following prior therapy with VEGF pathway inhibitors had stable disease at all sites for approximately 8 to 12 months, and a patient with PNET and liver metastases continued on the extension study for 18 months (36 doses) with stable disease.

However, the charge of the LNP must be taken into consideration. Cationic lipids are combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular Therapy, vol. 19, no. 12, pages 1286-2200, December 2011). Negatively charged polymers such as siRNA oligonucleotides may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). It has been shown that LNP siRNA systems containing these lipids exhibit remarkably different gene silencing properties in hepatocytes in vivo, with potencies varying according to the series DLinKC2-DMA>DLinKDMA>DLinDMA>>DLinDAP employing a Factor VII gene silencing model (see, e.g., Rosin et al, Molecular Therapy, vol. 19, no. 12, pages 1286-2200, December 2011). A dosage of 1 g/ml levels may be contemplated, especially for a formulation containing DLinKC2-DMA. Preparation of LNPs and IS110 encapsulation may be used/and or adapted from Rosin et al, Molecular Therapy, vol. 19, no. 12, pages 1286-2200, December 2011). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(w-methoxy-poly(ethylene glycol)2000) carbamoyl]-1,2-dimyristyloxlpropyl-3-amine (PEG-C-DOMG) may be provided by Tekmira Pharmaceuticals (Vancouver, Canada) or synthesized. The specific IS110 RNA may be encapsulated in LNPs containing DLinDAP, DLinDMA, DLinK-DMA, and DLinKC2-DMA (cationic lipid:DSPC:CHOL: PEGS-DMG or PEG-C-DOMG at 40:10:40:10 molar ratios). When required, 0.2% SP-DiOC18 (Invitrogen, Burlington, Canada) may be incorporated to assess cellular uptake, intracellular delivery, and biodistribution. Encapsulation may be performed by dissolving lipid mixtures comprising a cationic lipid:DSPC:cholesterol:PEG-c-DOMG (40:10:40:10 molar ratio) in ethanol to a final lipid concentration of 10 mmol/l. This ethanol solution of lipid may be added drop-wise to 50 mmol/l citrate, pH 4.0 to form multilamellar vesicles to produce a final concentration of 30% ethanol vol/vol. Large unilamellar vesicles may be formed following extrusion of multilamellar vesicles through two stacked 80 nm Nuclepore polycarbonate filters using the Extruder (Northern Lipids, Vancouver, Canada). Encapsulation may be achieved by adding RNA dissolved at 2 mg/ml in 50 mmol/l citrate, pH 4.0 containing 30% ethanol vol/vol drop-wise to extruded preformed large unilamellar vesicles and incubation at 31° C. for 30 minutes with constant mixing to a final RNA/lipid weight ratio of 0.06/1 wt/wt. Removal of ethanol and neutralization of formulation buffer were performed by dialysis against phosphate-buffered saline (PBS), pH 7.4 for 16 hours using Spectra/Por 2 regenerated cellulose dialysis membranes. Nanoparticle size distribution may be determined by dynamic light scattering using a NICOMP 370 particle sizer, the vesicle/intensity modes, and Gaussian fitting (Nicomp Particle Sizing, Santa Barbara, Calif.). The particle size for all three LNP systems may be ˜70 nm in diameter. siRNA encapsulation efficiency may be determined by removal of free siRNA using VivaPureD MiniH columns (Sartorius Stedim Biotech) from samples collected before and after dialysis. The encapsulated RNA may be extracted from the eluted nanoparticles and quantified at 260 nm. The siRNA to lipid ratio was determined by measurement of cholesterol content in vesicles using the Cholesterol E enzymatic assay from Wako Chemicals USA (Richmond, Va.). PEGylated liposomes (or LNPs) can also be used for delivery.

Preparation of large LNPs may be used/and or adapted from Rosin et al, Molecular Therapy, vol. 19, no. 12, pages 1286-2200, December 2011. A lipid premix solution (20.4 mg/ml total lipid concentration) may be prepared in ethanol containing DLinKC2-DMA, DSPC, and cholesterol at 50:10:38.5 molar ratios. Sodium acetate may be added to the lipid premix at a molar ratio of 0.75:1 (sodium acetate:DLinKC2-DMA). The lipids may be subsequently hydrated by combining the mixture with 1.85 volumes of citrate buffer (10 mmol/l, pH 3.0) with vigorous stirring, resulting in spontaneous liposome formation in aqueous buffer containing 35% ethanol. The liposome solution may be incubated at 37° C. to allow for time-dependent increase in particle size. Aliquots may be removed at various times during incubation to investigate changes in liposome size by dynamic light scattering (Zetasizer Nano ZS, Malvern Instruments, Worcestershire, UK). Once the desired particle size is achieved, an aqueous PEG lipid solution (stock=10 mg/ml PEG-DMG in 35% (vol/vol) ethanol) may be added to the liposome mixture to yield a final PEG molar concentration of 3.5% of total lipid. Upon addition of PEG-lipids, the liposomes should their size, effectively quenching further growth. RNA may then be added to the empty liposomes at an siRNA to total lipid ratio of approximately 1:10 (wt:wt), followed by incubation for 30 minutes at 37° C. to form loaded LNPs. The mixture may be subsequently dialyzed overnight in PBS and filtered with a 0.45-μm syringe filter.

Spherical Nucleic Acid (SNA™) constructs and other nanoparticles (particularly gold nanoparticles) are also contemplated as a means to deliver components of the IS110 transposase system to intended targets. Significant data show that Spherical Nucleic Acid (SNA™) constructs, based upon nucleic acid-functionalized gold nanoparticles, are superior to alternative platforms based on multiple key success factors, such as:

High in vivo stability. Due to their dense loading, a majority of cargo (DNA or siRNA) remains bound to the constructs inside cells, conferring nucleic acid stability and resistance to enzymatic degradation.

Deliverability. For all cell types studied (e.g., neurons, tumor cell lines, etc.) the constructs demonstrate a transfection efficiency of 99% with no need for carriers or transfection agents.

Therapeutic targeting. The unique target binding affinity and specificity of the constructs allow exquisite specificity for matched target sequences (i.e., limited off-target effects).

Superior efficacy. The constructs significantly outperform leading conventional transfection reagents (Lipofectamine 2000 and Cytofectin).

Low toxicity. The constructs can enter a variety of cultured cells, primary cells, and tissues with no apparent toxicity.

No significant immune response. The constructs elicit minimal changes in global gene expression as measured by whole-genome microarray studies and cytokine-specific protein assays.

Chemical tailorability. Any number of single or combinatorial agents (e.g., proteins, peptides, small molecules) can be used to tailor the surface of the constructs.

This platform for nucleic acid-based therapeutics may be applicable to numerous disease states, including inflammation and infectious disease, cancer, skin disorders and cardiovascular disease.

Citable literature includes: Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., Small, doi.org/10.1002/smll.201302143.

Self-assembling nanoparticles may be constructed with polyethyleneimine (PEI) that is PEGylated with an Arg-Gly-Asp (RGD) peptide ligand attached at the distal end of the polyethylene glycol (PEG), for example, as a means to target tumor neovasculature expressing integrins. Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. A dosage of about 50 to 500 mg of IS110 is envisioned for delivery in the self-assembling nanoparticles of Schiffelers et al.

The nanoplexes of Bartlett et al. (PNAS, Sep. 25, 2007, vol. 104, no. 39) may also be applied to the present invention. The nanoplexes of Bartlett et al. are prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. The DOTA-siRNA of Bartlett et al. was synthesized as follows: 1,4,7,10-tetraazacyclododecane-1,4,7,10-tetraacetic acid mono(N-hydroxysuccinimide ester) (DOTA-NHSester) was ordered from Macrocyclics (Dallas, Tex.). The amine modified RNA sense strand with a 100-fold molar excess of DOTA-NHS-ester in carbonate buffer (pH 9) was added to a microcentrifuge tube. The contents were reacted by stirring for 4 h at room temperature. The DOTA-RNAsense conjugate was ethanol-precipitated, resuspended in water, and annealed to the unmodified antisense strand to yield DOTA-siRNA. All liquids were pretreated with Chelex-100 (Bio-Rad, Hercules, Calif.) to remove trace metal contaminants. Tf-targeted and nontargeted siRNA nanoparticles may be formed by using cyclodextrin-containing polycations. Typically, nanoparticles were formed in water at a charge ratio of 3 (+/−) and an siRNA concentration of 0.5 g/liter. One percent of the adamantane-PEG molecules on the surface of the targeted nanoparticles were modified with Tf (adamantane-PEG-Tf). The nanoparticles were suspended in a 5% (wt/vol) glucose carrier solution for injection.

Davis et al. (Nature, Vol 464, 15 Apr. 2010) conducts a siRNA clinical trial that uses a targeted nanoparticle-delivery system (clinical trial registration number NCT00689065). Patients with solid cancers refractory to standard-of-care therapies are administered doses of targeted nanoparticles on days 1, 3, 8 and 10 of a 21-day cycle by a 30-min intravenous infusion. The nanoparticles consist of a synthetic delivery system containing: (1) a linear, cyclodextrin-based polymer (CDP), (2) a human transferrin protein (TF) targeting ligand displayed on the exterior of the nanoparticle to engage TF receptors (TFR) on the surface of the cancer cells, (3) a hydrophilic polymer (polyethylene glycol (PEG) used to promote nanoparticle stability in biological fluids), and (4) siRNA designed to reduce the expression of the RRM2 (sequence used in the clinic was previously denoted siR2B+5). The TFR has long been known to be upregulated in malignant cells, and RRM2 is an established anti-cancer target. These nanoparticles (clinical version denoted as CALAA-01) have been shown to be well tolerated in multi-dosing studies in non-human primates. Although a single patient with chronic myeloid leukemia has been administered siRNA by liposomal delivery, Davis et al.'s clinical trial is the initial human trial to systemically deliver siRNA with a targeted delivery system and to treat patients with solid cancer. To ascertain whether the targeted delivery system can provide effective delivery of functional siRNA to human tumours, Davis et al. investigated biopsies from three patients from three different dosing cohorts; patients A, B and C, all of whom had metastatic melanoma and received CALAA-01 doses of 18, 24 and 30 mg m⁻² siRNA, respectively. Similar doses may also be contemplated for the IS110 system of the present invention. The delivery of the invention may be achieved with nanoparticles containing a linear, cyclodextrin-based polymer (CDP), a human transferrin protein (TF) targeting ligand displayed on the exterior of the nanoparticle to engage TF receptors (TFR) on the surface of the cancer cells and/or a hydrophilic polymer (for example, polyethylene glycol (PEG) used to promote nanoparticle stability in biological fluids).

Delivery or administration according to the invention can be performed with liposomes. Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Liposomes can be made from several different types of lipids; however, phospholipids are most commonly used to generate liposomes as drug carriers. Although liposome formation is spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review).

Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. Further, liposomes are prepared from hydrogenated egg phosphatidylcholine or egg phosphatidylcholine, cholesterol, and dicetyl phosphate, and their mean vesicle sizes were adjusted to about 50 and 100 nm. (see, e.g., Spuch and Navarro, Journal of Drug Delivery, vol. 2011, Article ID 469679, 12 pages, 2011. doi:10.1155/2011/469679 for review).

Conventional liposome formulation mainly comprises natural phospholipids and lipids such as 1,2-distearoryl-sn-glycero-3-phosphatidyl choline (DSPC), sphingomyelin, egg phosphatidylcholines and monosialoganglioside. Since this formulation is made up of phospholipids only, liposomal formulations have encountered many challenges, one of the ones being the instability in plasma. Several attempts to overcome these challenges have been made, specifically in the manipulation of the lipid membrane. One of these attempts focused on the manipulation of cholesterol. Addition of cholesterol to conventional formulations reduces rapid release of the encapsulated bioactive compound into the plasma or 1,2-dioleoyl-sn-glycero-3-phosphoethanolamine (DOPE) increases the stability (see, e.g., Spuch and Navarro, Journal of Drug Delivery, vol. 2011, Article ID 469679, 12 pages, 2011. doi:10.1155/2011/469679 for review).

In a particularly advantageous embodiment, Trojan Horse liposomes (also known as Molecular Trojan Horses) are desirable and protocols may be found at cshprotocols.cshlp.org/content/2010/4/pdb.prot5407.long. These particles allow delivery of a transgene to the entire brain after an intravascular injection. Without being bound by limitation, it is believed that neutral lipid particles with specific antibodies conjugated to surface allow crossing of the blood brain barrier via endocytosis. Applicant postulates utilizing Trojan Horse Liposomes to deliver components of the IS110 transposase system to the brain via an intravascular injection, which would allow whole brain transgenic animals without the need for embryonic manipulation. About 1-5 g of nucleic acid molecule, e.g., DNA, RNA, may be contemplated for in vivo administration in liposomes.

In another embodiment, the components of the IS110 transposase system may be administered in liposomes, such as a stable nucleic-acid-lipid particle (SNALP) (see, e.g., Morrissey et al., Nature Biotechnology, Vol. 23, No. 8, August 2005). Daily intravenous injections of about 1, 3 or 5 mg/kg/day of a specific IS110 element targeted in a SNALP are contemplated. The daily treatment may be over about three days and then weekly for about five weeks. In another embodiment, a specific IS110 encapsulated SNALP) administered by intravenous injection to at doses of abpit 1 or 2.5 mg/kg are also contemplated (see, e.g., Zimmerman et al., Nature Letters, Vol. 441, 4 May 2006). The SNALP formulation may contain the lipids 3-N-[(wmethoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio (see, e.g., Zimmerman et al., Nature Letters, Vol. 441, 4 May 2006).

In another embodiment, stable nucleic-acid-lipid particles (SNALPs) have proven to be effective delivery molecules to highly vascularized HepG2-derived liver tumors but not in poorly vascularized HCT-116 derived liver tumors (see, e.g., Li, Gene Therapy (2012) 19, 775-780). The SNALP liposomes may be prepared by formulating D-Lin-DMA and PEG-C—DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulted SNALP liposomes are about 80-100 nm in size.

In yet another embodiment, a SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,Ndimethylaminopropane (see, e.g., Geisbert et al., Lancet 2010; 375: 1896-905). A dosage of about 2 mg/kg total IS110 element per dose administered as, for example, a bolus intravenous infusion may be contemplated.

In yet another embodiment, a SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA) (see, e.g., Judge, J. Clin. Invest. 119:661-673 (2009)). Formulations used for in vivo studies may comprise a final lipid/RNA mass ratio of about 9:1.

Other cationic lipids, such as amino lipid 2,2-dilinoleyl-4-dimethylaminoethyl-[1,3]-dioxolane (DLin-KC2-DMA) may be utilized to encapsulate components of the IS110 transposase similar to siRNA (see, e.g., Jayaraman, Angew. Chem. Int. Ed. 2012, 51, 8529-8533). A preformed vesicle with the following lipid composition may be contemplated: amino lipid, di stearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a nucleic acid/total lipid ratio of approximately 0.05 (w/w). To ensure a narrow particle size distribution in the range of 70-90 nm and a low polydispersity index of 0.11_0.04 (n=56), the particles may be extruded up to three times through 80 nm membranes prior to adding nucleic acid (e.g. bridgeRNA). Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

In some embodiments of the present disclosure, the system may be loaded into naturally occurring, engineered (e.g., rationally engineered), or adaptively evolved bacteriophage for delivery to microbial cell populations, e.g., endogenous microbial cells. Bacteriophages replicate within bacteria following the injection of their genome into the cytoplasm and do so using either a lytic cycle, which results in bacterial cell lysis, or a lysogenic (non-lytic) cycle, which leaves the bacterial cell intact. The bacteriophages of the present disclosure are, in some embodiments, non-lytic (also referred to as lysogenic or temperate). Non-lytic phage may also include those that are actively secreted from infected cells in the absence of lysis, including, without limitation, filamentous phage such as, for example, M13, fd, IKe, CTX-φ, Pf1, Pf2 and Pf3. Thus, after phage delivery to a bacterial cell, the bacterial cell may remain viable and able to stably maintain expression. In some embodiments, lytic bacteriophage may be used as delivery vehicles. When used with the system, naturally lytic phage serve as cargo shuttles and do not inherently lyse target cells.

Examples of non-lytic bacteriophage for use in accordance with the present disclosure include, without limitation, Myoviridae (P1-like viruses; P2-like viruses; Mu-like viruses; SPO1-like viruses; phiH-like viruses); Siphoviridae (λ-like viruses, γ-like viruses, T1-like viruses; T5-like viruses; c2-like viruses; L5-like viruses; psiM1-like viruses; phiC31-like viruses; N15-like viruses); Podoviridae (phi29-like viruses; P22-like viruses; N4-like viruses); Tectiviridae (Tectivirus); Corticoviridae (Corticovirus); Lipothrixviridae (Alphalipothrixvirus, Betalipothrixvirus, Gammalipothrixvirus, Deltalipothrixvirus); Plasmaviridae (Plasmavirus); Rudiviridae (Rudivirus); Fuselloviridae (Fusellovirus); Inoviridae (Inovirus, Plectrovirus, M13-like viruses, fd-like viruses); Microviridae (Microvirus, Spiromicrovirus, Bdellomicrovirus, Chlamydiamicrovirus); Leviviridae (Levivirus, Allolevivirus) and Cystoviridae (Cystovirus). Such phages may be naturally occurring or engineered phages. In some embodiments, the bacteriophage is a coliphage (e.g., infects Escherichia coli). In some embodiments, the bacteriophage of the present disclosure target bacteria other than Escherichia coli, including, without limitation, Bacteroides thetaiotamicron (e.g., B1), B. fragilis (e.g., ATCC 51477-B1, B40-8, Bf-1), B. caccae (e.g., phiHSC01), B. ovatus (e.g., phiHSC02), Clostridium difficile (e.g., phiC2, phiC5, phiC6, phiC8, phiCD 119, phiCD27), Klebsiella pneumoniae (e.g., KPO1K2, K11, Kpn5, KP34, JD001), Staphylococcus aureus (e.g., phiNMI, 80alpha), Enterococcus faecalis (e.g., IME-EF1), Enterococcus faecium (e.g., ENB6, C33), and Pseudomonas aeruginosa (e.g., phiKMV, PAK-P1, LKD16, LKA1, delta, sigma-1, J-1). Other bacteriophage maybe used in accordance with the present disclosure.

### H. Methods of Predicting Target and Donor Efficiency

Described herein is a method of predicting target and donor efficiency for any given IS110 transposase. In some embodiments, the method is used to predict the efficiency of target and donor site sequences in a genome of interest. In some embodiments, the genome is a eukaryotic genome. In some embodiments, the genome is the human genome. In some embodiments, the method is used to predict the efficiency of target and donor site sequences for orthologs of a given IS110 transposase.

In some embodiments, the method comprises training a neural network model with target and donor sequences and measured efficiency data for a given IS110 transposase to generate a trained neural network model. In some embodiments, the efficiency data is from a screen performed in a first species. In some embodiments, the first species is E. coli. In some embodiments, the target and donor input sequences are about 9 bases in length. In some embodiments, the target and donor input sequences do not include a core sequence.

In some embodiments, the method further comprises applying the trained neural network model to a genome sequence of a second species to generate efficiency predictions for target and donor sequences of the genome. In some embodiments the second species is a eukaryote. In some embodiments, the second species is a human. In some embodiments, disclosed herein is a nucleic acid editing system as described throughout wherein the bridgeRNA targets a donor and target sequence with the best predicted efficiency for the given IS110 transposase.

The invention is further described by the following non-limiting Examples.

### EXAMPLES

Examples are provided below to facilitate a more complete understanding of the invention. The following examples serve to illustrate the exemplary modes of making and practicing the invention. However, the scope of the invention is not to be construed as limited to specific embodiments disclosed in these Examples, which are illustrative only.

### Example 1: Materials and Methods

This example describes the materials and methods used by the applicants to obtain the results shown in subsequent examples.

Analysis of LE and RE lengths across IS110 elements. Sequence coordinate information about individual IS elements was collected through the ISfinder web portal (Siguier et al. 2006). This included information about the total length of each IS element, as well as the start and end coordinates of the transposase CDS. The LE non-coding length was calculated from the CDS coordinates for each IS110 element as the distance between the 5′ terminus and start of the CDS, and the RE non-coding length was calculated as the distance between the end of the CDS and the 3′ terminus.

Metagenomic and genomic sequence database. A database was constructed from publicly-available metagenomic and isolated sequencing data, as described previously (Wei et al., n.d.). Briefly, a custom sequence database of bacterial isolate and metagenomic sequences was constructed by aggregating publicly available sequence database, including NCBI, UHGG (Almeida et al. 2021), JGI IMG (Chen et al. 2021), the Gut Phage Database (Camarillo-Guerrero et al. 2021), the Human Gastrointestinal Bacteria Genome Collection (Forster et al. 2019), MGnify (Mitchell et al. 2020), Youngblut et al animal gut metagenomes (Youngblut et al. 2020), MGRAST (Meyer et al. 2008), and Tara Oceans samples (Sunagawa et al. 2015). The final sequence database included 37,067 metagenomic samples, 274,880 bacterial and archaeal metagenome-assembled genomes (MAGs), 855,228 bacterial and archaeal isolate genome samples, and 185,140 predicted viral genome samples.

Annotating IS110 transposase coding sequences. Genomic sequences were annotated using Prodigal (Hyatt et al. 2010) to identify coding sequences (CDS). All unique protein sequences were then combined into a single FASTA file and clustered at 30% sequence identity using mmseqs2 (Steinegger and Soding 2017). Two Pfam domains found in IS110 transposases—DEDD_Tnp_IS110 (PF01548) and Transposase_20 (PF02371)—were used to search against these clustered representative proteins using the hmmsearch tool in the hmmer package (Finn, Clements, and Eddy 2011). Candidates between 250 and 500 aa in length were retained for further analysis. All members of the retained 30% identity clusters were then extracted, and the same IS110 Pfam domain significance thresholds were applied to these candidates to generate a final list of candidate IS110 transposases.

Phylogenetic analysis of IS110 transposases. Given this set of transposase protein sequences, we further curated it for phylogenetic analysis. First, all protein sequences with ambiguous residues were removed. Next, hmmsearch was rerun with the parameters -Z 1000000-E 1e−3 using hmmsearch and the Pfam domains DEDD_Tnp_IS110 (PF01548) and Transposase_20 (PF02371). Next, only protein sequences that contained one match to both domains, and where the DEDD_Tnp_IS110 domain preceded the Transposase_20, were retained. Next, this filtered set of protein sequences was clustered at 90% identity across 85% of the aligned sequence using the mmseqs2 easy-cluster algorithm. Next, protein sequences were filtered such that only proteins that contained a DEDD_Tnp_IS110 RuvC-like domain that was between 130 and 170 amino acids in length, and a Transposase_20 Tnp domain that was between 75 and 103 amino acids in length, were retained. Next, using the identified 90% protein sequence clusters, a representative from each cluster was selected that was closest to the 80th percentile in total length. This resulted in a curated set of 90% identity cluster representatives. Next, 90% identity cluster representatives were clustered at 40% identity across 70% of the aligned sequences using the mmseqs2 easy-cluster algorithm. These clusters were then used to select 40% identity cluster representatives, of which there were 1,323. Next, an initial protein sequence alignment was performed on these cluster representatives using mafft command with --maxiterate 2. Next, representative RuvC, coiled-coil, and Tnp domains were identified from the initial alignments to determine the boundaries of these domains within the alignment, with the coiled-coil being defined as the intervening sequence between the two Pfam domains. These three domains were then extracted from each sequence in the alignment as separate sequence files and individually aligned using the mafft-linsi --maxiterate 1000 --reorder command. These alignments were then concatenated according to protein sequence of origin, and filtered to exclude columns with over 75% gaps. Pairwise percent identity was calculated for all of the members of the resulting alignment, and a graph network was constructed of all members using 50% identity as a minimum cutoff for each undirected edge between proteins. All connected components were then identified, and only one member per component was retained, prioritized members with the fewest overall gaps. This resulted in a final alignment with 1,250 protein sequences. A phylogenetic tree was then constructed using iqtree2 v2.1.4-beta, with all default parameters except -T 32 (Minh et al. 2020). Additional metadata about each sequence was mapped onto the tree, including host kingdom and phylum, ISfinder group, and notable orthologs.

Predicting IS110 element boundaries. To identify the boundaries of each element, an initial search was conducted using comparative genomics to identify putative pre-insertion and post-insertion examples within the custom sequence database. IS110 protein candidates were clustered at 30% identity using mmseqs2 (Steinegger and Soding 2017), and within each cluster all relevant genomic loci were identified. Nucleotide sequences were then extracted from the database by adding 1,000 base pairs to the 5′ and 3′ ends of the IS110 CDS, and extracting the complete intervening sequence. If examples did not contain enough flanking sequence, they were excluded. These extracted sequences were then referred to as a “locus” in the singular and “loci” in the plural. IS110 loci were then separated into “batches” based on 90% identity protein clusters. These batches were then searched against up to 40 metagenomic or isolate samples in the custom database, prioritizing samples that already contained related transposases. Putative pre-insertion sites were identified if the distal ends of the loci aligned by BLAST to a contiguous sequence (Altschul et al. 1990), but the IS110 CDS did not. Precise boundaries of the IS110 element were then predicted using a modified method similar to what was implemented by the previously published tool MGEfinder (Durrant et al. 2020). Core sequences were identified as repeated sequences near the end of the predicted element. This search resulted in thousands of diverse loci with predicted IS110 element boundaries.

Next, an iterative BLAST search was used to extend IS110 element boundary predictions beyond those that could be detected by identifying pre-insertion sites. IS110 elements were searched using BLAST against all IS110 loci. Hits were retained only if both ends of the element aligned, and if the core was concordant between query and target. This then generated a new set of IS110 elements and their boundaries, which were recycled as query sequences, and the search was repeated for another iteration. This repeated for 36 iterations before convergence (no new IS110 elements were found). The combined set of IS110 boundaries were kept for further analysis.

Structural alignment to identify bridgeRNA consensus structure. A pipeline was developed to identify conserved RNA structures in the sequences immediately flanking the transposase CDS. First, the IS621 protein sequence was searched against the complete IS110 database for orthologs using blastp and the parameters -max target_seqs 1000000-evalue 1e−6. Only hits that were at least 30% identical at the amino acid level with 80% of both sequences covered by the alignment were retained. Up to 2000 unique proteins were then selected in order of descending percent amino acid identity. Flanking sequences for the corresponding proteins were then retrieved from the database, with flanking sequences defined as a 5′ flank of up to 255 bp (including 50 bp of 5′ CDS) and a 3′ flank of up to 170 bp (including 50 bp of the 3′ CDS). These flanks were then further filtered to exclude sequences that were more than 35 bases shorter than the target flank lengths. Sequences were filtered to exclude those with ambiguous nucleotides. Protein sequences were then clustered using mmseqs2 easy-linclust with a minimum percent nucleotide identity cutoff of 95% across 80% of the aligned sequences, and one set of flanks for each representative was retained. Flanking sequences were then clustered at 90% nucleotide identity across 80% of the aligned sequences, and only one representative flanking sequence pair per cluster was retained. Then, up to 200 sequences were selected in order of decreasing percent identity shared between the IS621 protein sequence and their corresponding ortholog protein sequence. The remaining sequences were then individually analyzed for secondary RNA structures using linearfold (Huang et al. 2019). Sequences were then aligned to each other using the mafft-qinsi alignment algorithm and parameter --maxiterate 1000. Alignment columns with over 50% gaps were removed. Conserved RNA secondary structure was then projected onto the alignment, and manually inspected to nominate bridgeRNA boundaries. This region was exported as a separate sequence alignment file, and a consensus RNA secondary structure was predicted using ConsAlifold (Tagashira and Asai 2022). This structure was then visualized using R2R (Weinberg and Breaker 2011). This same pipeline was used to analyze hundreds of other IS110 elements, resulting in diverse secondary structures such as those displayed in FIG. 13. These consensus structures were converted into covariance models using infernal, and these were then searched across thousands of sequences to nominate bridgeRNA boundaries (Nawrocki and Eddy 2013).

Nucleotide covariation analysis to identify bridgeRNA guide sequences. To identify programmable guide sequences in the bridgeRNA of the IS621 element, the following approach was taken. First, the IS621 protein sequence was searched against our collection of IS110 transposase proteins with predicted element boundaries using blastp. Next, only alignments that met a cutoff of 20% amino acid identity across 90% of both sequences were retained. Next, a covariance model (CM) of the bridgeRNA secondary and primary sequence was used to identify homologs of the bridgeRNA sequence in the non-coding ends of these orthologous sequences (Nawrocki and Eddy 2013). 50 nucleotide target and donor sequences were extracted centered around the core. For elements with multiple predicted boundaries, boundaries with a CT dinucleotide core were prioritized. Next, elements that were identified at earlier iterations in our boundary search were prioritized. Next, elements that were similar in length to the known IS621 sequence element were prioritized. Only 1 element per unique locus was retained. Predicted bridgeRNA sequences were then aligned using the cmalign tool in the Infernal package (Nawrocki and Eddy 2013). FASTA sequence files were generated that contained concatenated target and bridgeRNA sequences, and concatenated donor and bridgeRNA sequences. This alignment was then further filtered to remove all columns that contained gaps in the IS621 bridgeRNA sequence. These alignments were then analyzed using CCMpred to identify co-varying nucleotides between target/donor and bridgeRNA sequences (Ekeberg et al. 2013). These covariation scores were visualized as a heatmap, and inspected for patterns of linear covariation within the predicted bridgeRNA sequences. Possible base-pairing interactions were identified within the two internal loops of the bridgeRNA, leading to the proposed model for bridgeRNA target/donor recognition. The same covariation analysis was performed on the donor alone, leading to a theory for possible non-programmable STIR sequences.

This covariation analysis was combined with a base-pairing analysis to better identify the DNA strand that was being bound by the bridgeRNA, if any. This was accomplished using a permutation test based on the same alignment that was used as input into the covariation analysis. First, for each pair of columns in the target/donor alignment and the bridgeRNA alignment, the observed base-pairing concordance was calculated by taking the sum of non-gap rows in one column that matched the non-gap rows in the second column. To determine a null distribution for this estimate, 1,000 random permutations of these columns were performed and the base-pairing concordance was re-calculated. The mean score and the standard deviation of this permuted score distribution was calculated. This was then used to calculate a Z-score for the observed base-pairing concordance. This same procedure was repeated, but for the complement of the ncRNA nucleotides. The maximum score of the two base-pairing concordance Z-scores was used, with complement base-pairing scores being then assigned a negative value. This sign of this final score was then multiplied by the covariation score to better visualize possible base-pairing patterns.

Small RNA sequencing of IS110 bridgeRNAs. RNA was isolated from cells encoding plasmids bearing a RE-core-LE or RE-LE sequence after growth overnight on a LB agar plate with appropriate antibiotics to retain the plasmid bearing the RE-core-LE. RNA isolation was performed using Direct-zol RNA Miniprep Kit (Zymo). RNA was prepared for small RNA sequencing according to the following protocol. Briefly, no more than 5 pg total RNA was treated with DNase I (NEB) for 30 minutes at 37° C. then purified using RNA Clean & Concentrator -5 Kit. Ribosomal RNA was depleted from samples using Ribo-Zero Plus rRNA Depletion Kit (Illumina) and purified using RNA Clean & Concentrator—5 Kit. Depleted RNA was treated with T4 PNK for six hours at 37° C., supplementing with T4 PNK and ATP after six hours for one additional hour. RNA was purified using RNA Clean & Concentrator—5 kit and subsequently treated with RNA 5′ Polyphosphatase (Lucigen) for 30 minutes at 37° C. RNA was purified with RNA Clean & Concentrator—5 Kit and concentration was measured via nanodrop. Next-generation sequencing libraries were prepared using NEBNext Multiplex Small RNA Library Prep Kit (NEB) according to the manufacturer's protocol. Resultant libraries were sequenced on an Illumina MiSeq using a 2×150 Reagent Kit v2.

Analysis of small RNA sequencing data. Demultiplexed fastq files were cleaned and merged using bbduk and bbmerge, respectively (Bushnell, Rood, and Singer 2017). Merged fastq files were aligned to the RE-Core-LE bearing plasmid using bwa mem (Li and Durbin 2009). Signal to noise ratio was represented as the percentage of all reads aligned to the RE-Core-LE plasmid that were mapped to the RE-Core-LE donor sequence.

IS110 plasmid transposition assay in E. coli. BL21 DE3 cells (NEB) were co-transformed with one plasmid encoding a target site and the IS110 IS621 transposase and a second plasmid encoding a bridgeRNA, a donor site sequence, and a GFP upstream of the donor site sequence such that upon recombination with the target site GFP expression would be activated by a synthetic promoter adjacent to the target site. In some cases, the bridgeRNA is encoded within an RE-core-LE. In some cases, the bridgeRNA is expressed from a synthetic promoter. In some cases, the bridgeRNA encodes specificity for the WT target site sequence and WT donor site sequence. In some cases, the bridgeRNA encodes specificity for sequences other than the WT sequence for both the target site sequence and donor site sequence via reprogramming of the target binding loop and donor binding loop of the bridgeRNA. In some cases, the bridgeRNA and transposase are expressed from one plasmid, while the donor site sequence and target site sequence are oriented appropriately to a GFP coding sequence on a second plasmid such that excisive recombination or inversion mediated by the transposase and bridgeRNA results in GFP expression. Co-transformed cells were plated on LB agar containing kanamycin, chloramphenicol, and 0.07 mM IPTG. Plates were incubated at 37° C. for 16 hours and subsequently incubated at room temperature for 8 hours. Several colonies that appeared to express GFP under blue light were seeded in TB containing kanamycin and chloramphenicol and incubated for 16 hours at 37° C. shaking at 200 rpm. Plasmid-plasmid integration product plasmids were purified using QIAprep Spin Miniprep Kit and sent for whole plasmid sequencing to confirm integration product sequence (Primordium Labs). Hundreds of colonies were subsequently scraped from the plate, resuspended in TB, and diluted to an appropriate concentration for flow cytometry. 50000 cells were analyzed on a Novocyte Quanteon Flow Cytometer to assess the percentage of GFP expressing cells.

IS110 pooled target screen design. A pooled screen was designed to test target loop mismatch tolerance and relative efficiency across diverse guide sequences. Several categories of oligos were designed to answer different questions. First, 10,656 oligos were designed to test hundreds of different target guides with single mismatch pairs. That is, for a given target, one position in the guide and the corresponding position in the target to generate all 4×4=16 combinations of nucleotides. Target guides were selected to reduce genomic off-targets. Next, 3,600 oligos were designed to test different combinations of double mismatches between target loop and target. Next, 2,135 oligos were designed to test the boundaries and programmability of the guide outside of the known programmable sequences, to determine if increased specificity is possible. Next, 2,000 oligos were designed as an internal set of negative controls by ensuring that none of the 9 programmable positions (excluding the CT core) matched in the target loop and target. Next, another 1,800 oligos were designed to test more single mismatch combinations, but did not include all 4×4 combinations in target and target loop. Next, 1,610 oligos were designed to test how mismatches in the dinucleotide core of the bridgeRNA sequences affected recombination efficiency. Finally, the GO1 positive control was included for comparison. One unique barcode per amplicon was assigned at random, ensuring that no 2 barcodes were within 2 mismatches of each other. Each oligo encoded a target site sequence, a promoter to activate expression of a reporter molecule after recombination of a donor site sequence with the target, a barcode to identify the target-target loop pair, a promoter to express a bridgeRNA, and a target loop of a bridgeRNA. The oligo was flanked on both ends with sequences suitable for golden gate cloning into a desired plasmid backbone. All oligos were ordered as a single pooled library from Twist.

IS110 pooled target screen experimental protocol. The library of target-bridgeRNA pairs was cloned into a plasmid encoding a T7-inducible IS110 transposase such that a full length bridgeRNA was reconstituted in the plasmid. The bridgeRNA donor loop was encoded to bind to the WT donor site sequence. The library of plasmids encoding the target-bridgeRNA pairs was co-electroporated into BL21 DE3 cells with a second plasmid encoding a WT donor site sequence adjacent to a kanamycin resistance gene such the kanamycin gene would be transcribed upon recombination between the two plasmids. After co-electroporation and recovery, cells were plated on bioassay dishes with LB agar. One plating condition, serving as the control, was LB agar with chloramphenicol and ampicillin, which maintain the plasmids but do not induce or require recombination. A second condition was LB agar with chloramphenicol, ampicillin, kanamycin, and 0.1 mM IPTG; IPTG induces transposase expression, prompting recombination, while kanamycin selects for cells that have induced recombination between the donor and target plasmid. Both conditions were performed in two replicates. Recombination indicates a compatible target-target loop pair within the library.

IS110 pooled target screen sequencing preparation. Hundreds of thousands of colonies were scraped from the bioassay dishes and had plasmid DNA extracted using Nucleobond Xtra Midiprep Kit (Macherey Nagel). After plasmid DNA isolation, samples were prepared for next generation sequencing. For DNA isolated from the control conditions, a PCR was used to amplify the barcodes specifying target and bridgeRNA pairs to measure the distribution of barcodes without selecting conditions. For DNA isolated from selection conditions, a PCR was used to amplify the barcodes specifying target and bridgeRNA pairs, with one primer priming from the donor plasmid and the other priming from the target plasmid such that only barcodes from recombinant plasmids were measured. The distributions of barcodes from recombinant plasmids was subsequently compared to the distribution of barcodes under control conditions.

IS110 pooled target screen analysis. Amplicon sequences were processed using the bbduk tool (Bushnell, Rood, and Singer 2017). Amplicon sequencing data was then aligned to their respective wildtypes using bwa mem, with ambiguous nucleotides at all variable positions. Barcodes were then extracted from the amplicons using custom python scripts. Barcodes were mapped to the designed barcode library, tolerating single mismatches when making assignments. This resulted in a table of barcode counts per biological replicate. Using custom R scripts, the counts were normalized within each replicate using counts per million (CPM), which converts raw barcode counts into barcode counts per million barcodes. CPM values were then averaged across the two biological replicates in each condition. For the recombinant barcodes, CPM values were then corrected by the control barcode CPM values using a simple correction factor for each barcode, calculated by dividing the expected barcode CPM (under a uniform distribution) by the observed barcode CPM. These corrected CPM values were subsequently used in many of the individual analyses. Mismatch tolerance was assessed by limiting the analysis to the top quintile of most efficient 4×4 single mismatch sets, and then averaging the percentage of total CPM within each set at each position. The motif of enriched nucleotides at each position was generated by determining the nucleotide composition of the top quintile of most efficient target loop/target pairs (without mismatches), and comparing this to the nucleotide composition of the entire set. All oligos were ordered as a single pooled library from Twist.

IS110 pooled donor screen design. A pooled screen was designed to test donor loop programmability, mismatch tolerance, and relative efficiency across diverse guide sequences. Several categories of oligos were designed to answer different questions. Donor sequences were selected to reduce predicted genomic off-targets. First, 13,593 oligos were designed that included complete single mismatch scans across 100 distinct donors, including all position 4×4=16 mismatches with the donor at the corresponding position. Next, 5000 completely random donor guides were selected and paired with a perfect match donor for the analysis of a high number of diverse donor sequences. Next, 2,297 oligos to test single mismatch and double mismatch scans of the WT donor sequence and 4 other functional donors were included. Next, 50 negative control oligos were included that ensured that none of the 9 programmable positions (excluding the CT core) matched in the donor loop and donor. Finally, 30 oligos included mutations where the WT donor bridgeRNA promoter box sequences were mutated to determine their effect on efficiency. Each oligo encoded a partial RE, a donor site sequence, and full length LE encoding a bridgeRNA as found in the WT system such that expression of the bridgeRNA would be mediated by the natural system promoter. The donor site sequence and donor loop sequence of the bridgeRNA were modified in each member according to the description supra, while the target loop of the bridgeRNA was constant and programmed to recognize a target site sequence not found in the BL21 DE3 E. coli genome. The oligo was flanked on both ends with sequences suitable for golden gate cloning into a desired plasmid backbone. All oligos were ordered as a single pooled library from Twist.

IS110 pooled donor screen experimental protocol. The library of donor-bridgeRNA pairs was cloned into a library of backbone plasmids encoding a partial RE adjacent to a kanamycin gene such that the full length RE was reconstituted, such that the kanamycin gene would be transcribed upon recombination between the donor plasmid and a second plasmid encoding a target, and such that a unique molecular identifier (UMI) within the library of backbone plasmids was adjacent to the donor-bridgeRNA pair. The library of plasmids encoding the donor-bridgeRNA pairs was co-electroporated into BL21 DE3 cells with a second plasmid encoding a E. coli genome orthogonal target sequence adjacent to a constitutive promoter and encoding a T7-inducible IS110 transposase. After co-electroporation and recovery, cells were plated on bioassay dishes with LB agar. One plating condition, serving as the control, was LB agar with chloramphenicol and ampicillin, which maintain the plasmids but do not induce or require recombination. A second condition was LB agar with chloramphenicol, ampicillin, kanamycin, and 0.07 mM IPTG; IPTG induces transposase expression, prompting recombination, while kanamycin selects for cells that have induced recombination between the donor and target plasmid. Both conditions were performed in two replicates. Recombination indicates a compatible donor-donor loop pair within the library.

IS110 pooled donor screen sequencing preparation. Hundreds of thousands of colonies were scraped from the bioassay dishes and had plasmid DNA extracted using Nucleobond Xtra Midiprep Kit (Macherey Nagel). After plasmid DNA isolation, samples were prepared for next generation sequencing. For DNA isolated from the control conditions, a PCR was used to amplify the UMI specifying donor and bridgeRNA pairs to measure the distribution of UMIs without selecting conditions. For DNA isolated from selection conditions, a PCR was used to amplify the UMIs specifying donor and bridgeRNA pairs, with one primer priming from the donor plasmid and the other priming from the target plasmid such that only UMIs from recombinant plasmids were measured. The distributions of UMIs from recombinant plasmids was subsequently compared to the distribution of UMIs under control conditions. UMIs were initially mapped to donor-bridgeRNA pairs by amplifying a region of the input donor library such that the information of all variable sites within the full length of the RE-LE were captured in addition to the adjacent UMI.

IS110 pooled donor screen analysis. First, All amplicon sequence data was pre-processed using bbduk to remove adapters. Next, unique molecular identifiers (UMIs) were mapped to their respective oligos. This was done by aligning to the expected amplicon sequence with ambiguous N nucleotides in all the variable positions using bwa mem. UMIs were then determined from the alignments, and combined with the variable LDG and RDG to guarantee the uniqueness of each UMI to each oligo. Next, control and recombinant samples were analyzed in much the same way as the previously described target screen, but UMIs were counted rather than assigned barcodes. Next, UMI counts were converted to CPM, averaged across two biological replicates, and normalized according to the correction factors calculated in the control condition. These CPM values were then analyzed across different oligo categories to assess mismatch tolerance, how distance from the wild-type donor affects efficiency, and what nucleotide sequences were favored/disfavored at each position in the donor.

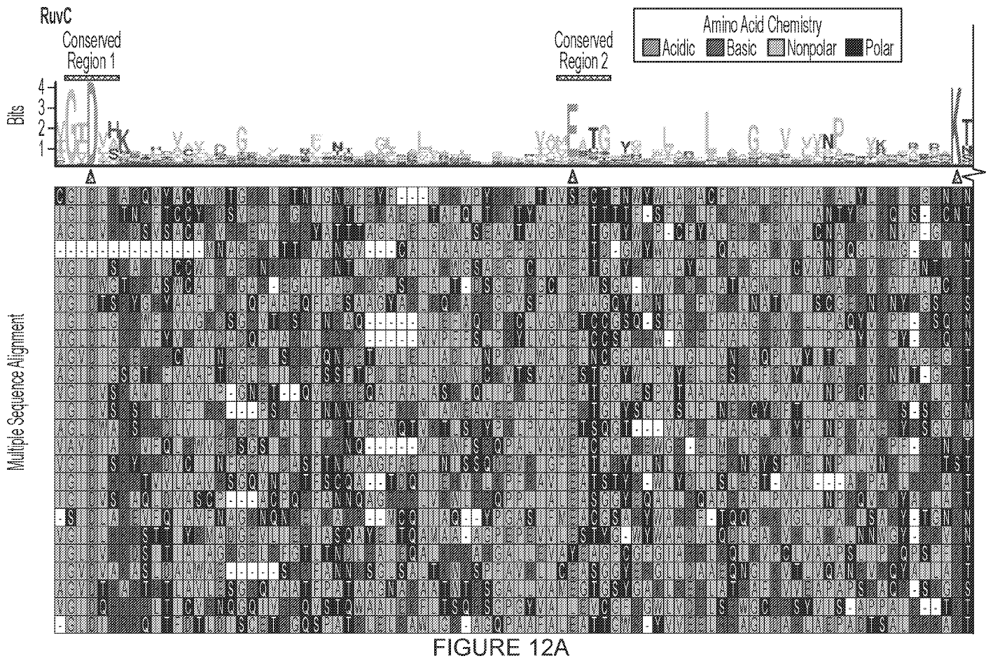

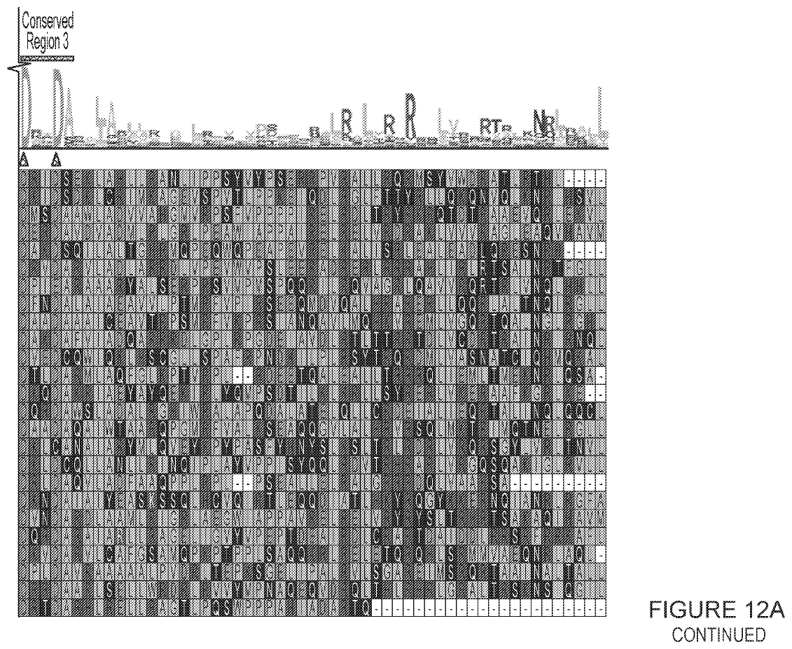

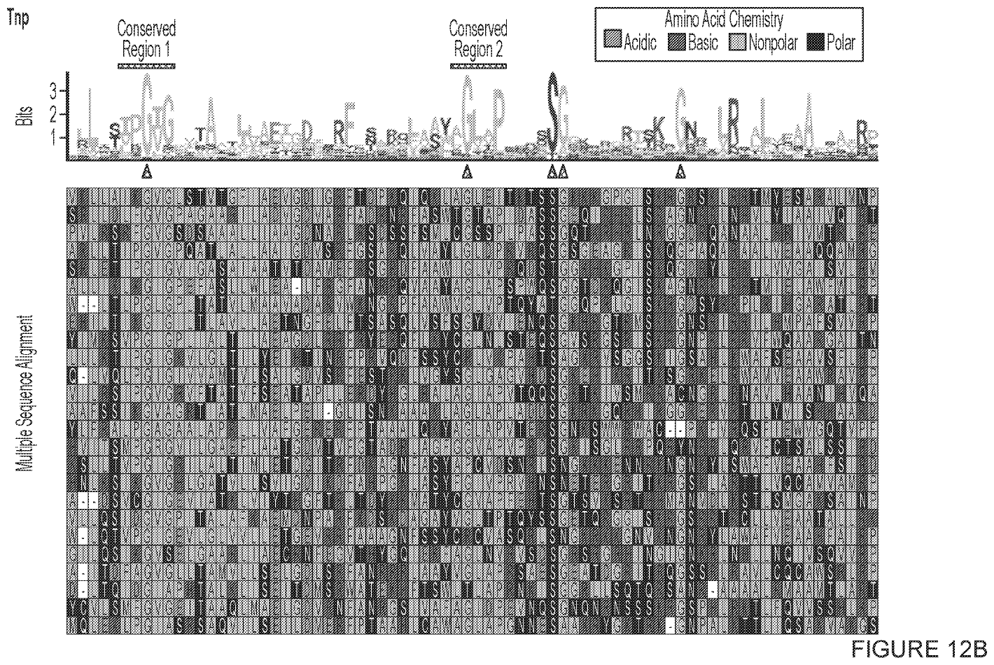

Analysis of conserved residues and regions in IS110 family transposase domains. Applicants took the following approach to identify regions of functional significance within IS110 transposase protein sequences. Pre-defined IS110 profile HIM4M models DEDD_Tnp_IS110 (PF01548) and Transposase_20 (PF02371) were searched against a collection of 1,453,592 putative IS110 transposases using hmmsearch (Finn, Clements, and Eddy 2011). Regions that were identified by the DEDD_Tnp_IS110 model were extracted from each sequence as predicted RuvC domains, and filtered by the Applicants according to an E-value cutoff of 1e−3 and a length cutoff of greater than 124 and less than 176 amino acids. Regions that were identified by the Transposase_20 model were extracted from each sequence as predicted Tnp domains, and filtered by the Applicants according to an E-value cutoff of 1e−3 and a length cutoff of greater than 59 and less than 111 amino acids. Applicants aligned extracted domains using the hmmalign tool (Finn, Clements, and Eddy 2011) clustered extracted domains using mmseqs2 (Steinegger and Soding 2017) at 90% sequence identity. One domain representative of each 90% identity cluster was then used to identify conserved residues and regions, with preference given to residues with known catalytic activity. Applicants further analyzed these conserved residues and regions to identify the domain motifs presented in FIGS. 21-24. Applicants also visualized the conserved residues and regions in FIGS. 12A-12B.

IS110 genomic insertion assay with long-read nanopore sequencing. A plasmid was prepared encoding a donor site sequence adjacent to a constitutively expressed kanamycin resistance gene and a temperature sensitive Rep101 protein. Plasmid replication of this donor plasmid was eliminated in cells upon growth at 37° C., ensuring that cells encode a single copy of the donor plasmid. A cell line was prepared encoding this donor plasmid by transforming BL21 DE3 and making the resultant cell line chemically competent using Mix & Go preparation kit (Zymo). The temperature sensitive donor plasmid was then transformed with a pHelper plasmid encoding a T7-inducible transposase and a constitutively expressed bridgeRNA. The donor loop of the bridgeRNA was programmed to recognize the donor site sequence within the donor plasmid and the target loop of the bridgeRNA was programmed to recognize a target site sequence in the BL21 DE3 E. coli genome. After transformation, cells were recovered and plated on 10 cm LB agar plates with chloramphenicol to retain the pHelper plasmid and kanamycin to require integration of the donor plasmid into the genome for cell survival. The 1000s of resultant colonies, each with an integration of the donor plasmid into the genome, were scraped from the plate. Genomic DNA was extracted from the pool of cells using Quick DNA Miniprep plus kit (Zymo). Genomic DNA was then cleaned up using AMpure XP (Beckman Coulter) and sequenced using Oxford Nanopore Technologies nanopore sequencing to at least 100× genome coverage.

Analysis of IS110 genomic insertion nanopore sequencing data. To identify long reads containing potential insertion junctions between plasmid donor and E. coli genome, we first scanned all individual reads for the presence of the terminal 20 nucleotides of the donor sequence, excluding the core. If a 20 base-pair sub-sequence of a read matched the 5′ terminus or 3′ terminus (allowing for up to 2 mismatches), then the read was split and the flanking sequences were written to separate files. These flanking sequences were then mapped back to the plasmid sequences and the E. coli genome using minimap2 (Li 2018), and assigned as originating from the plasmid or the E. coli genome according to whichever had the higher alignment score. Reads were then assigned to specific insertion junctions in the human genome to identify precise insertion sites. Insertion sites that were within 5 base-pairs of each other were merged together using bedtools merge (Quinlan and Hall 2010) and a representative insertion site was selected. The total number of unique reads per insertion site was selected, and the relative ratios of these read abundances were calculated to analyze genomic insertion specificity.

Analysis of IS110 transposase tertiary structure. AlphaFold models for thousands of IS110 transposase sequences are publicly available through the AlphaFold Protein Structure Database (AFDB; alphafold.ebi.ac.uk). This database was searched to identify all protein sequences that had the terms “IS110” or “IS1111” in their UniProt descriptions. 40,512 such sequences were identified and downloaded from AFDB. The IS110 domain pHMMs (DEDD_Tnp_IS110 and Transposase_20) were searched against the primary sequences of this collection of structures using hmmsearch and the parameters “—Z 1000000-E 10.” Only pHMM matches with an e-value less than 1e−3 were retained. The DEDD_Tnp_IS110 was required to be between 125 and 175 amino acids in length, and the Transposase_20 was required to be between 60 and 110 amino acids in length. Only proteins that had both of the domains after applying these filters were retained, resulting in 24,043 predicted IS110 protein structures. Protein sequences were then clustered using MMseqs2 command “easy-cluster -c 0.8 --threads 8 --cluster-reassign”, first with the “--min-seq-id 0.90” parameter on the unique sequences, and then with the “--min-seq-id 0.50” parameter on the 90% identity clusters. The TM-align software (v20220412; Zhang and Skolnick 2005) was used to determine the TM-scores of all the protein structures with respect to the IS621 AlphaFold protein structure. Substructures were extracted for comparison using the biopython python package. Distances between conserved nucleotides were calculated using the biopython package. This process was repeated for IS630 transposase to determine the specificity of TM-score as a cutoff for identifying related orthologs.

In vitro transcription of bridgeRNAs. In vitro transcription was carried out on a linear DNA template using the HighScribe T7 High Yield RNA Synthesis Kit (New England Biolabs) as per the manufacturer's instructions. DNA template was prepared by cloning into a pUC19 backbone and plasmid was linearized using SapI restriction enzyme (NEB) and purified using DNA Clean & Concentrate (Zymogen). After IVT, RNA was purified using the Monarch RNA Cleanup Kit. Where necessary, bridgeRNA was further purified denaturing polyacrylamide gel electrophoresis, extracted from the gel using UV shadowing and recovered by ethanol precipitation.

Microscale Thermophoresis. Microscale thermophoresis (MST) was carried out using a Monolith NT.1 I5pico Series instrument (NanoTemper technologies). IS621 recombinase was labeled for MST using the RED-MALEIMIDE 2nd Generation cysteine reactive kit (NanoTemper technologies) as per the manufacturer's instructions. Labeled protein was eluted in a buffer containing 20 mM Tris-HCl, 500 mM NaCl, 5 mM MgCl2, 1 mM DTT, 0.01% Tween20, pH 7.5. In order to determine affinity of recombinase for RNA, 20 nM recombinase were incubated with a dilution series (2500-0.076 nM) of purified LE encoded ncRNA or a scrambled RNA of equivalent length. MST was performed at 37° C. using premium capillaries (NanoTemper) at 30% LED excitation and medium MST power. Data were analyzed using the NanoTemper MO. affinity analysis software package and raw data were plotted on Prism for visualization. The binding affinity of IS621 RNP for donor and target DNA, as well as donor and target DNA containing scrambled LD-RD and LT-RT regions were determined using the MST tertiary binding function. Single-strand DNA was purchased from IDT (Coralville, USA) and annealed in buffer containing 10 mM Tris pH 8.0, 5 mM MgCl2 and 5 mM KCl. For MST, 20 nM RNP consisting of labeled IS621 recombinase and LE encoded ncRNA were incubated with a dilution series of duplexed donor or target DNA oligonucleotides (10 μM to 0.076 nM). MST was performed at 37° C. using premium capillaries (NanoTemper) at medium MST power with the LED excitation-power set to automatic (excitation ranged from 20-50%).

Predicting target and donor efficiency. Using target and donor screen data efficiency data, neural net models were constructed to predict the efficiency of unseen targets and donors. The variable 9 nt target and donor sequences (excluding the 2 nt core) were used as input into the models. Efficiency was measured as log 10(CPM+1). The efficiency data was split into training and test datasets, with 10% of the data used as a test dataset. Fully connected neural net models were constructed and tested using the Keras python package (Chollet and Others 2015). A range of random hyperparameter permutations were tested using KerasTuner (O'Malley et al. 2019), including different numbers of layers (1, 2, or 3), and nodes (256, 512, 1024, and 2,048), and different loss function (mean squared error and mean absolute error) and final layer activation functions (ReLU and linear). The model that performed best on a validation dataset (20% of the training dataset) across 40 random hyperparameter permutations was selected for both target and donor efficiency prediction. This model was then retrained for 100 epochs, and the epoch that minimized the validation loss was selected as the final model. Efficiency predictions were made for all 262,144 target and donor sequences and stored in a single file. The best performing models were then run on the held-out test dataset, and correlations between predicted efficiency and observed experimental efficiency were quite high (r=0.828 for target screen, r=0.847 for donor screen, P<2.2e−16 for both correlation tests).

### Example 2: Description of General Features of IS110 Elements

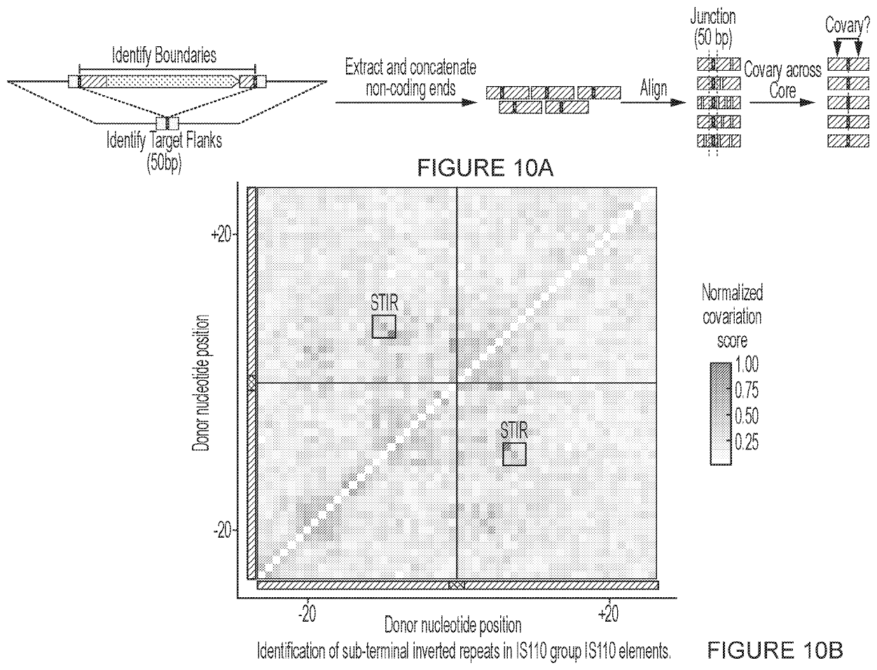

Based on literature examples, applicants identified that IS110 elements are split into two groups, an IS110 and IS1111 group. Applicants noted that both groups comprise an LE, transposase, and RE; they also noted that IS110s also encode a core sequence found at either end of the element (FIG. 1A). Members of the IS110 group were known to encode sub-terminal inverted repeats (STIRs) previously, while applicants identified that members of the I110 group also encode short STIRs (FIG. 10B). I110 transposases encode protein domains that are RuvC-like with a canonical DEDD catalytic motif, they also encode a transposase domain with a catalytic serine (FIG. 1). IS110 elements were previously known to undergo cut-and-paste recombination, excising from an integration site to form a circular form where the RE and LE are concatenated (FIG. 1C). Formation of a promoter at the RE-LE junction was also a known phenomenon of IS110s. To further characterize IS110s, applicants identified thousands of IS110 transposases and built a phylogenetic tree from their primary sequences; applicants mapped known IS110 elements onto this phylogeny and noted the host kingdom and phylum of the element, indicating broad distribution (FIG. 1D). Applicants analyzed the non-coding end length of IS110 and IS1111 groups of IS110s listed in the public ISFinder database, identifying that IS110s typically have longer LEs and IS111 s have longer REs (FIG. 1E).

### Example 3: Identification of the bridgeRNA

Using IS621 and closely related orthologs, applicants performed RNAseq on cells expressing an RNA from the LE via the natural promoter. RNAseq analysis indicated that transcription of an RNA corresponds with the transcription start site of the known sigma70 promoter motif, and that the resultant RNA spans the remainder of the length of the LE of the IS110 (FIG. 2A). This RNA was named the bridgeRNA. Using microscale thermophoresis, applicants demonstrated that purified IS621 transposase specifically binds the bridgeRNA; an accessory structure at the 5′ end of the bridgeRNAwas not found to be required for binding but did increase affinity for the bridgeRNA when present (FIG. 2B). An alignment of LEs across many orthologs was subsequently analyzed for patterns in RNA secondary structure, assuming the presence of bridgeRNAs within LEs of distant orthologs of IS621 (FIG. 2C). A consensus RNA structure indicated the presence of a 5′ stem-loop followed by two additional stem-loops with internal loop regions (FIG. 2D).

### Example 4: Prediction and Verification of the Mechanism of bridgeRNA Recognition of Donor and Target Site Sequences

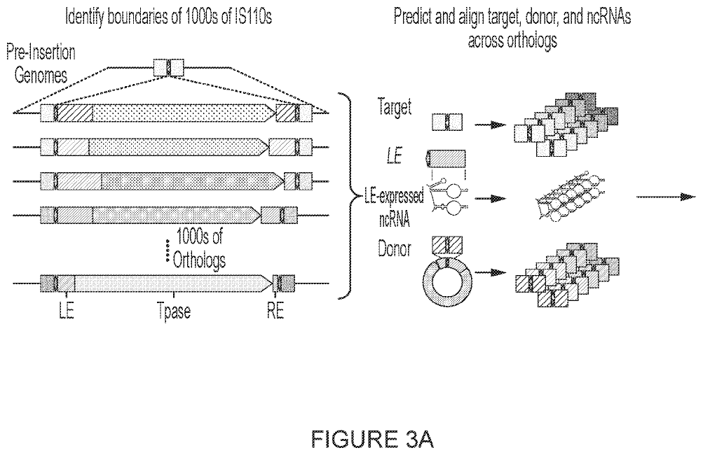

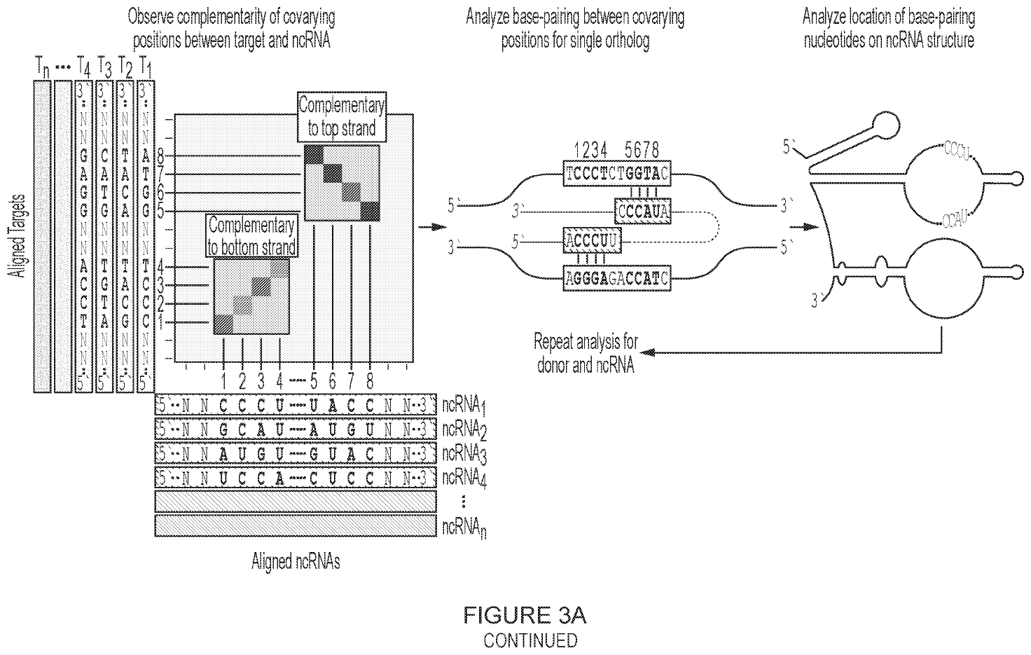

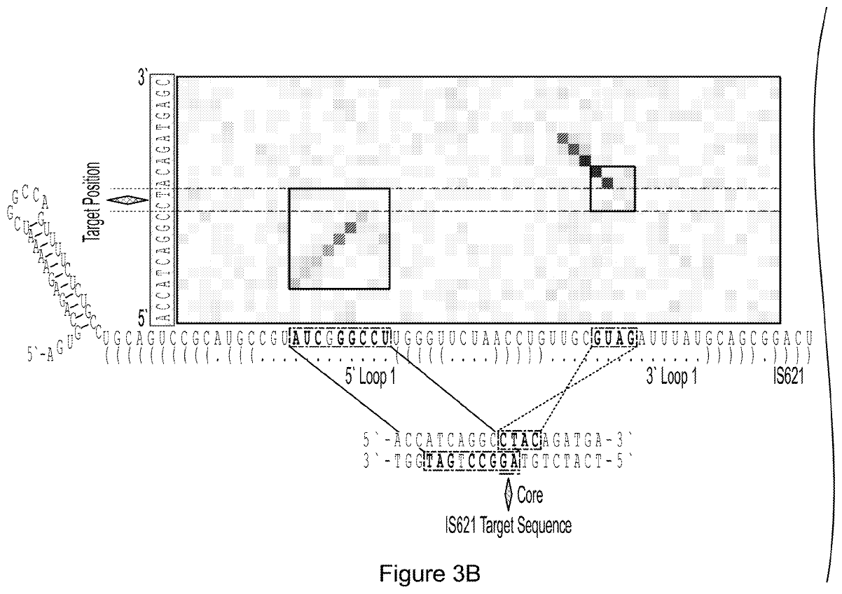

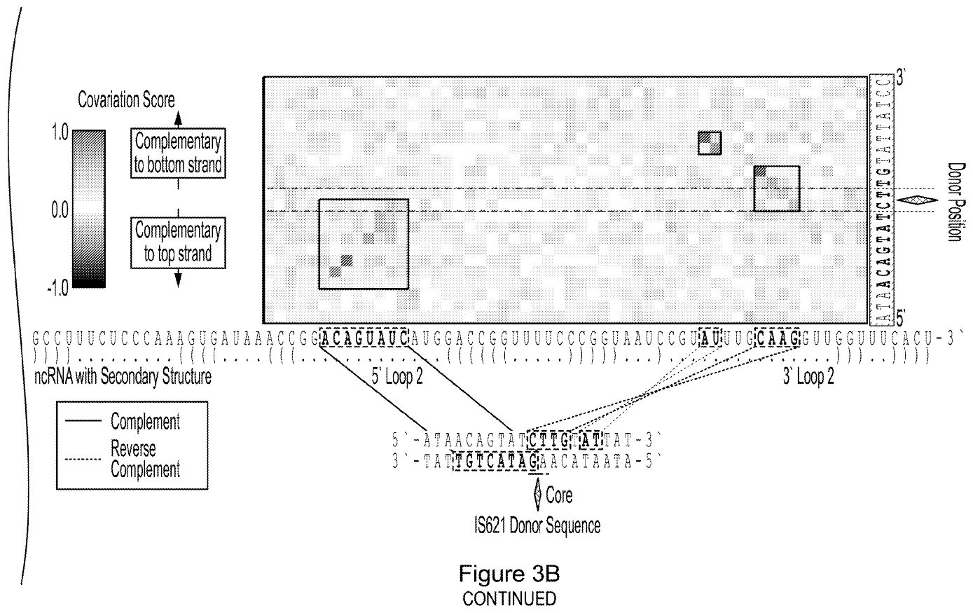

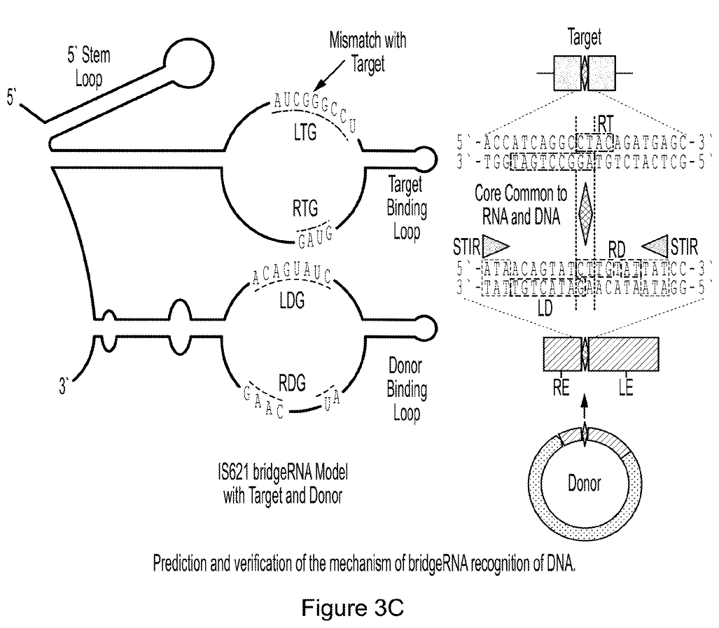

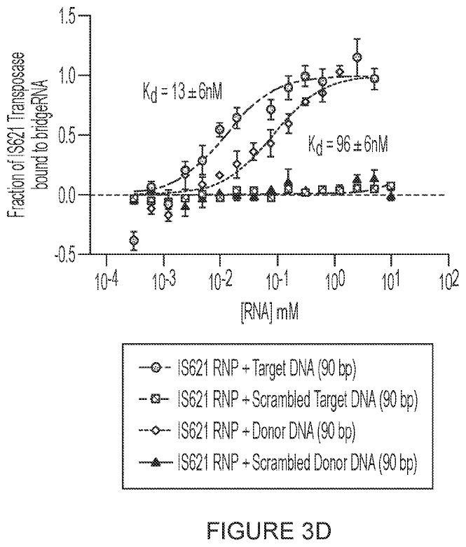

Using comparative genomics and other computational techniques, the target site sequences, predicted bridgeRNA boundaries, and donor site sequences of thousands of IS110 elements were identified and extracted for subsequent alignment to identify covarying bases between the bridgeRNA sequence and both the donor and target (FIG. 3A). Applicants identified two primary regions of the bridgeRNA that covary with the target and two primary regions of the bridgeRNA that covary with the donor across diverse IS110 orthologs (FIG. 3B). Upon inspection, there was evidence of potential base-pairing between these covarying regions of the bridgeRNA and the target and donor (FIG. 3B). Using the consensus bridgeRNA structure identified in FIG. 2D, Applicant's identified the location of these potential base-pairing sites within the two loops of the bridgeRNA, named the target binding loop and donor binding loop (FIG. 3C). Using purified IS621 transposase and bridgeRNA, applicants measured binding affinity of the purified RNP to the predicted donor and target site sequences using microscale thermophoresis, indicating that the RNP binds the WT target and donor sequences in a base-pairing specific manner (FIG. 3D).

### Example 5: Method for Reprogramming bridgeRNAs Using IS621 as an Example

For a given target and donor site sequence, the LTG, RTG, LDG, and RDG can be reprogrammed to specifically base pair with subsequences of the target site sequence and donor site sequence. The bases in the target and donor can be any base; examples where the cores match are shown but matching cores may not be required between the target and donor. Additionally, the STIR is not strictly required for donor site sequences (FIG. 4A).

### Example 6: Demonstration of Transposition in Cellulo Using Components of the IS110 System

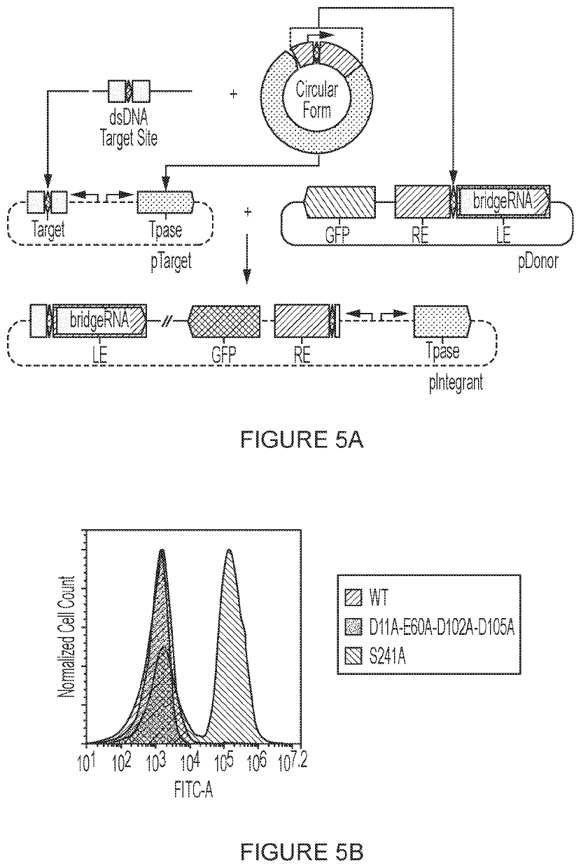

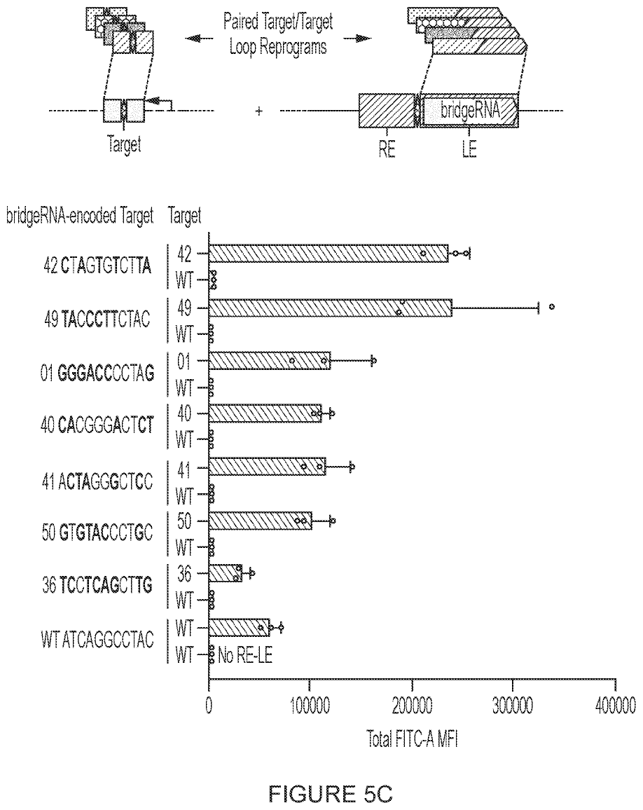

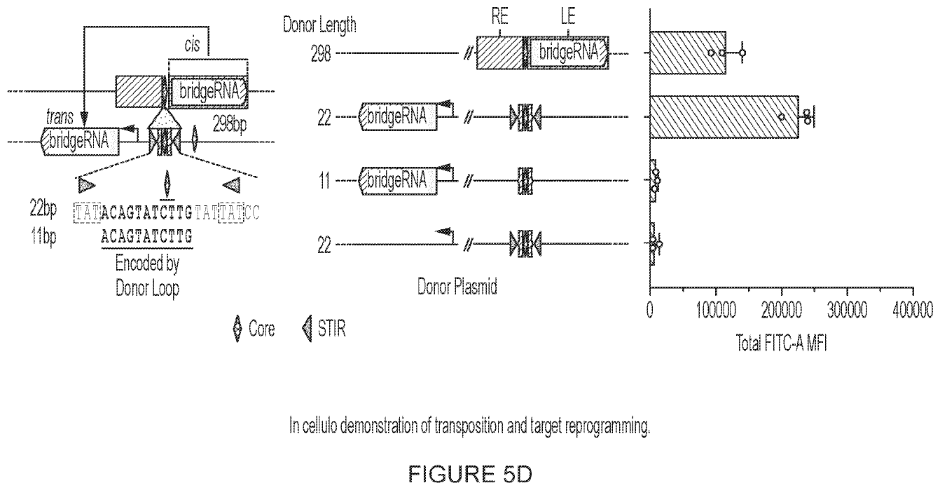

Applicants designed an assay to measure transposition by encoding the RE-LE donor junction, target site sequence, and transposase on plasmids with a GFP reporter (FIG. 5A, see EXAMPLE 1). To demonstrate the effectiveness of this system, Applicants measured GFP expression when using a WT IS621 transposase and compared it with a catalytically inactivated IS621 transposase by mutating the RuvC domain or the Tnp domain, showing that GFP expression was only observed when the WT transposase was provided (FIG. 5B). Using bridgeRNA reprogramming strategies previously described, the bridgeRNA was reprogrammed within the LE to encode a target loop specific for a new target. Pairing this new target loop with a target site sequence with which it could base pair resulted in transposition, while pairing with the IS621 WT target site sequence resulted in no observed transposition (FIG. 5C). Applicants further demonstrated that the bridgeRNA can be expressed from a synthetic promoter by truncating the RE-LE and measuring transposition efficiency (FIG. 5D). Applicants note that in this case provision of the 11 bp donor site sequence reduced transposition efficiency near the background, yet some transposition events were confirmed by sequencing of transposition products.

### Example 7: IS621 bridgeRNA Target/Target Loop High-Throughput Screen

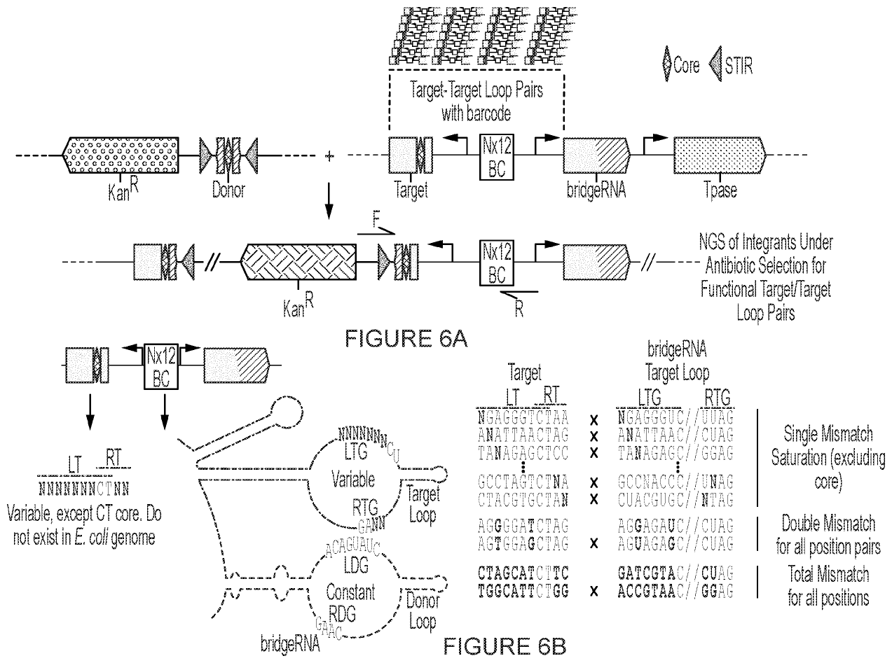

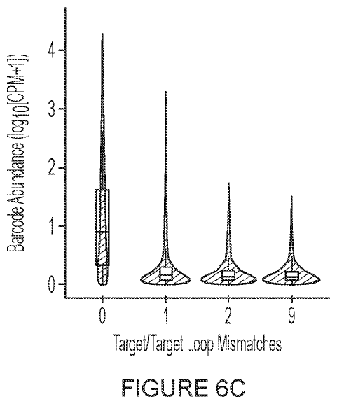

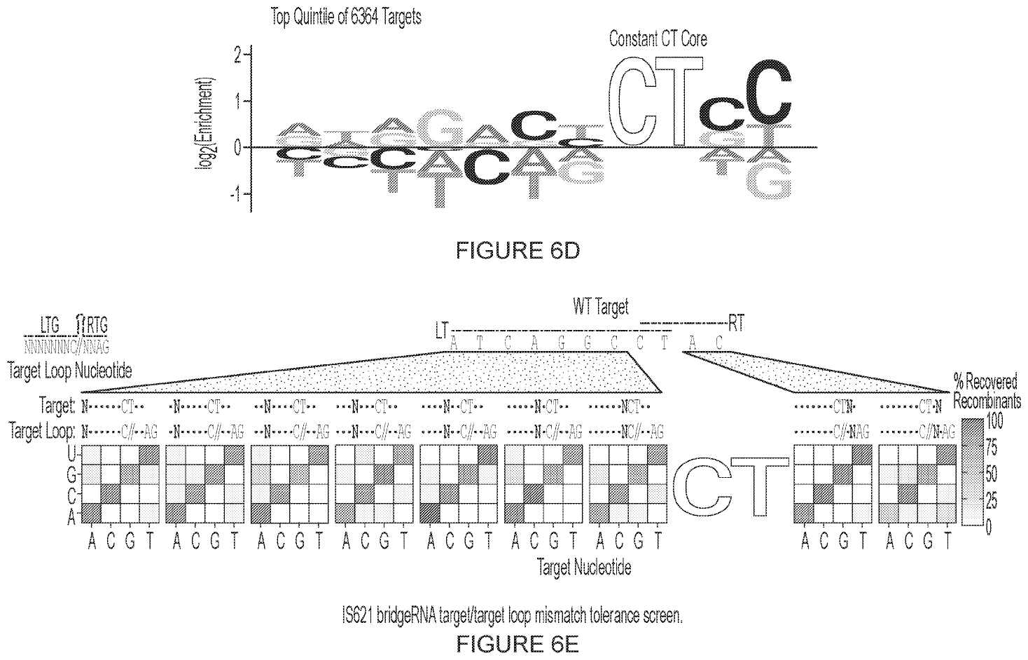

To explore the characteristics of bridgeRNA target loop reprogramming, Applicants designed a selection and next-generation sequencing based high-throughput screen (FIG. 6A-6B, see EXAMPLE 1). Applicants identified that the target loop is sensitive to single mismatches with the target, and very sensitive to double mismatches with the target, with a similar distribution of barcode counts per million (CPM) to target/target loop pairs that have 9 mismatches (FIG. 6C). From the screen data, applicants identified a motif for the top 20% most efficient target/target loop pairs (FIG. 6D). Applicants also observed the effect of mismatches at each position of the target/target loop pair, noting that most positions strongly prefer matches over mismatches (FIG. 6E).

### Example 8: IS621 bridgeRNA Donor/Donor Loop High-Throughput Screen

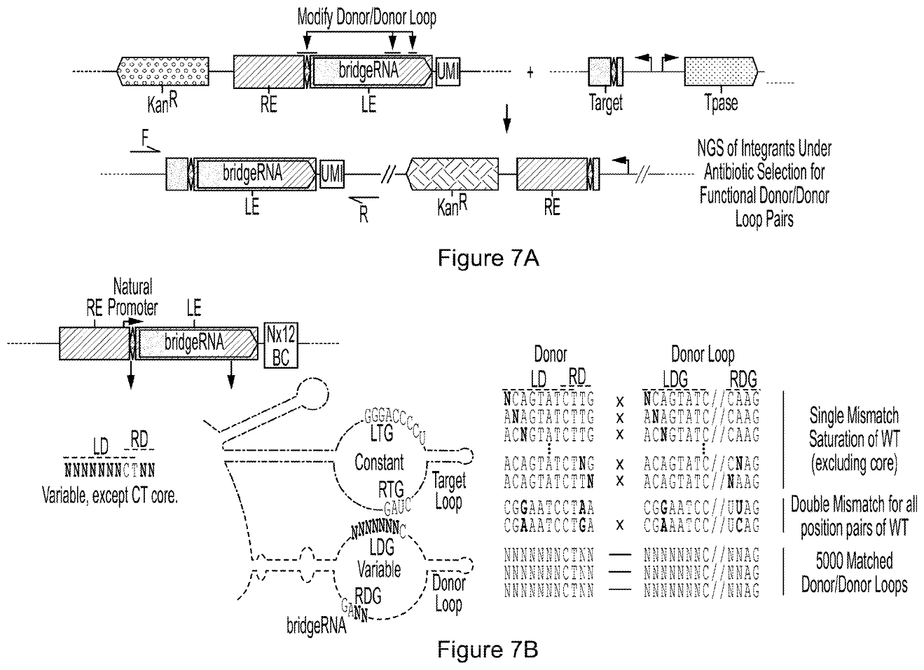

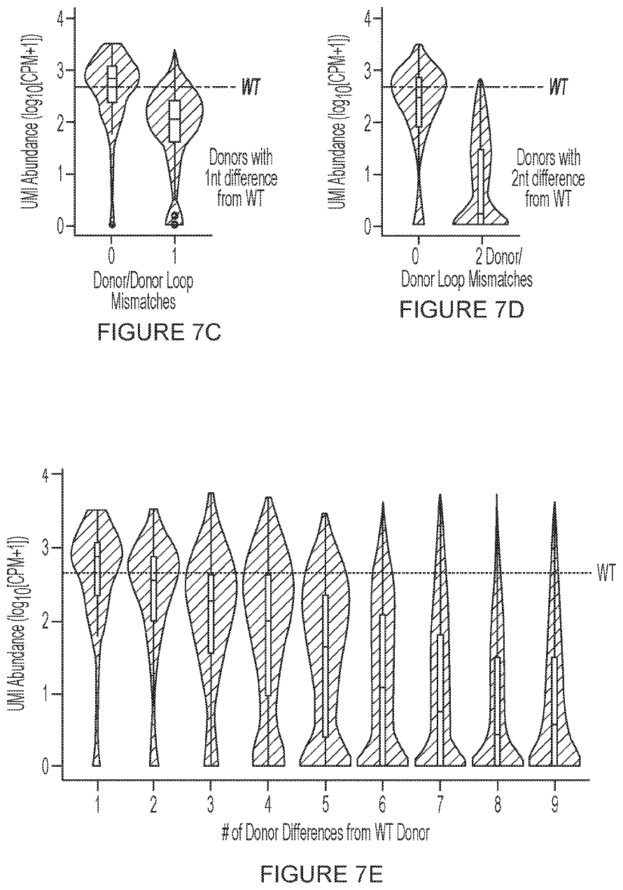

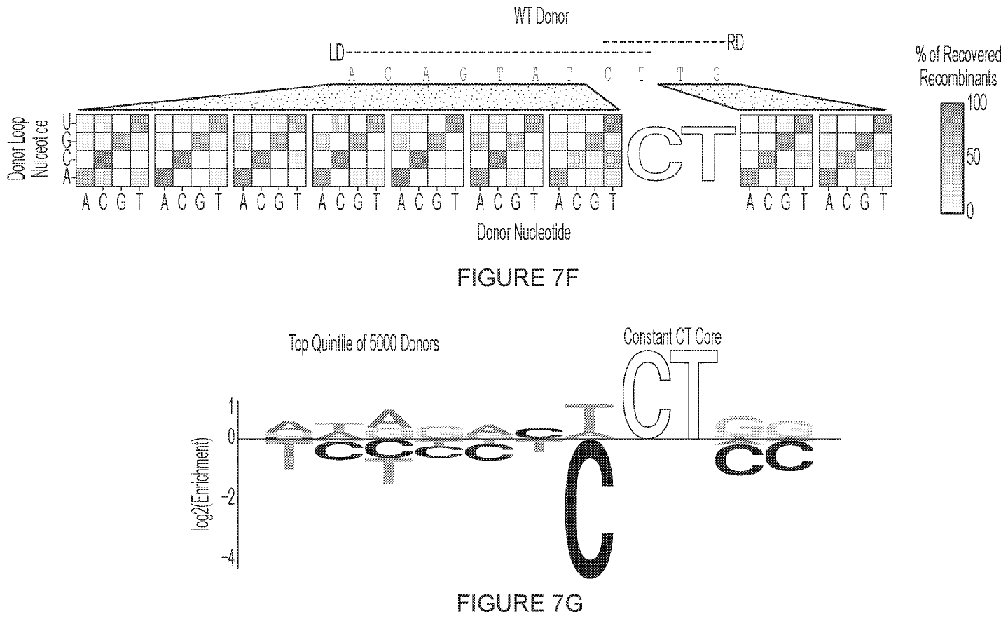

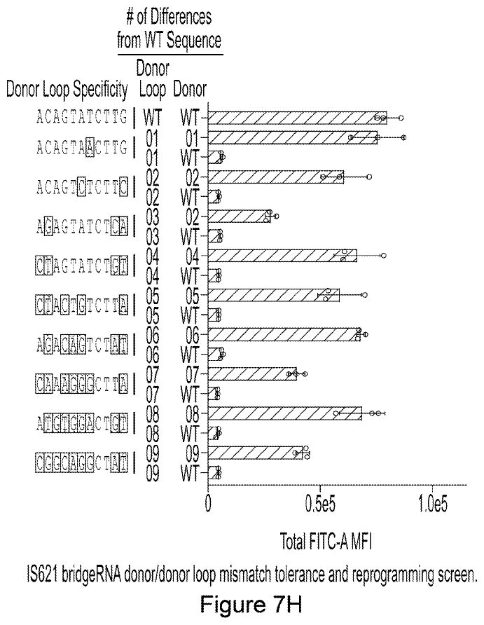

To explore the characteristics of bridgeRNA donor loop reprogramming, Applicants designed a selection and next-generation sequencing based high-throughput screen (FIG. 7A-7B, see EXAMPLE 1). Applicants compared the WT donor site sequence to donors with 1 or 2 nucleotide differences from the WT donor, noting that the WT donor is not the most efficient donor sequence tested (FIG. 7C-7D). Applicants also noted that single mismatches between the donor and donor loop were sometimes tolerated, while double mismatches between the donor and donor loop were largely not tolerated (FIG. 7C-7D). Across all donor and donor loop pairs tested, Applicants measured the relative efficiency of donors differing by up to 9 bases from the WT donor; efficient donors were identified with any number of differences from the WT, indicating complete reprogrammability (FIG. 7E). Applicants also observed the effect of mismatches at each position of the donor/donor loop (FIG. 7F). From the screen data, Applicants identified a motif for the top 20% most efficient donor/donor loop pairs (FIG. 7G). Using the GFP reporter assay depicted in FIG. 5D, Applicants measured transposition efficiency of reprogrammed donors with a varying number of differences from WT, indicating broad reprogrammability (FIG. 7H).

### Example 9: Comparison of Recombination Efficiency Using Reporters for Insertion, Excisive Recombination, and Inversion

Applicants designed GFP reporter assays to assess and compare recombination efficiency for insertion, excisive recombination, and inversion, finding similar recombination efficiency across reaction types (FIG. 8A-8C, see EXAMPLE 1).

### Example 10: Integration of Large Cargoes into E. coli Genomes Using Reprogrammed bridgeRNAs

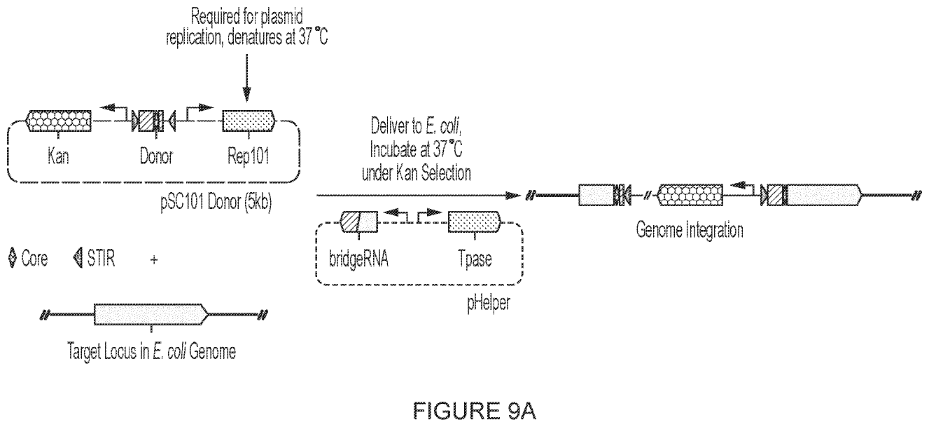

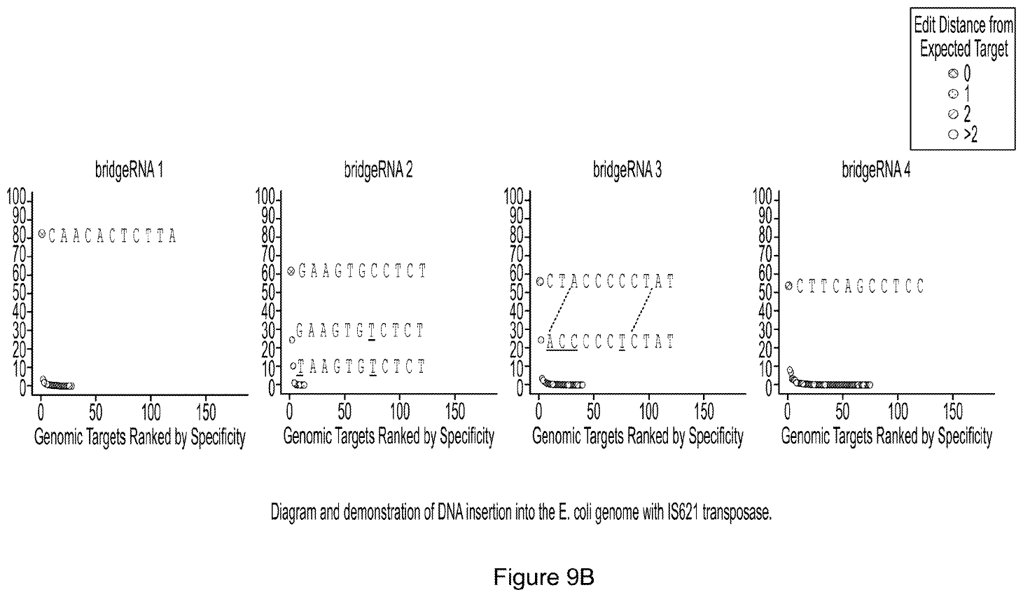

Applicants developed an approach for integrating single donor molecules into the E. coli genome using a bridgeRNA and the IS621 transposase (FIG. 9A, see EXAMPLE 1). Using this approach, applicants evaluated 4 bridgeRNAs programmed to direct integration of the donor plasmid into specific target site sequences in the E. coli genome. After analysis with nanopore whole genome sequencing, applicants demonstrate that the donor is primarily integrated into the programmed site, with some additional off-target integration sites that could be explained by high similarity with the intended target. (FIG. 9B).

### Example 11: Identification of Sub-Terminal Inverted Repeat Sequences in IS110s Using Covariation Analysis

Applicants used a covariation analysis approach to interrogate covarying bases between the RE and LE over the RE-LE or RE-core-LE (FIG. 10A, see EXAMPLE 1). Covariation analysis revealed the presence of sub-terminal inverted repeats across diverse orthologs (FIG. 10B).

### Example 12: Prediction and Verification of a bridgeRNA from an IS1111 Element

Applicants used analysis of conserved structures within the RE of an IS1111 to identify a putative bridgeRNA structure (FIG. 11A). Applicants subsequently built a consensus structure model of the bridgeRNA using ConsAliFold and R2R, and detected expression of the bridgeRNA from the RE in cellulo (FIG. 111B-11C, see EXAMPLE 1).

### Example 13: Alignment of RuvC-Like and Tnp Domains of Diverse IS110 Transposases

Applicants identified three conserved regions that consist of the known catalytic residues and other conserved residues within the RuvC domain of IS110 transposases (FIG. 12A). Applicants identified two conserved regions in addition to several other conserved residues within the Tnp domain of IS110 transposases, including a catalytic serine residue (FIG. 12B).

### Example 14: Diverse bridgeRNA Structures Associated with Diverse IS110 Transposases

Applicants identified clusters of bridgeRNA structures across diverse IS110 elements (see EXAMPLE 1). Applicants depicted some of these structures using R2R, and indicated the name of a representative IS110 element associated with each bridgeRNA structure (FIG. 13).

### Example 15: IS110 Protein Structure Analysis

Recent advances in the prediction of tertiary protein structures from primary sequences enable new techniques for identifying homologous protein sequences. The previous state-of-the-art in homology detection included protein sequence alignment or profile hidden markov models (pHMM) of known protein domains. Previously, scientists have chosen some sequence similarity cutoff, such as a percent amino acid identity above 90%, to assign proteins to the same functional family. Here, we used the AlphaFold deep learning model to predict protein structure and thereby to identify proteins that are homologous at the level of their 3-dimensional structure, which can be more sensitive for detecting more distant evolutionary relationships (van Kempen et al. 2023). This Example demonstrates that a TM-score cutoff of greater than 0.5 is very sensitive and specific when it comes to identifying members of the IS110 family, and it can be used to identify related transposases of similar molecular function. This Example also demonstrates that conserved residues in IS110 structures consistently appear within protein structures at similar distances to each other, again providing evidence for conserved function.

### Protein Tertiary Structure Similarity

Rather than percent amino acid identity, a different metric is needed to assess the similarity of two protein structures. A widely used and robust similarity metric for this purpose is called the Template modeling score (TM-score), defined as the formula shown in FIG. 14A.

### IS110 Protein Structure Analysis

Since the introduction of AlphaFold, protein structure prediction has dramatically accelerated. Large collections of predicted structures have been made publicly available, including the AlphaFold Protein Structure Database (AFDB; alphafold.ebi.ac.uk). This database contains over 200 million predicted protein structures spanning a wide diversity of protein families.

This database was searched to identify all protein sequences that had the terms “IS110” or “IS1111” in their UniProt descriptions. 40,512 such sequences were identified and downloaded from AFDB. The IS110 domain pHEMMs (DEDD_Tnp_IS110 and Transposase_20) were searched against the primary sequences of this collection of structures using hmmsearch and the parameters “—Z 1000000-E 10.” Only pHEMM matches with an e-value less than 1e−3 were retained. We required that the DEDD_Tnp_IS110 were between 125 and 175 amino acids in length, and that the Transposase_20 matches were between 60 and 110 amino acids in length. Only proteins that had both of the domains after applying these filters were retained, resulting in 24,043 predicted IS110 protein structures. The TM-align software (v20220412) was used to determine the TM-scores of all the protein structures with respect to the IS621 transposase AlphaFold protein structure. The distribution of TM-scores is shown in FIG. 14B.

The vast majority of predicted IS110 protein structures have a high TM-score when compared with the predicted IS621 structure. When using scores that are normalized by the average length of the query protein sequence and the IS621 protein sequence, 99.7% exceed a TM-score cutoff of 0.5, 99.4% exceed a TM-score cutoff of 0.6, 88.1% exceed a TM-score cutoff of 0.7, and 18% exceed a TM-score cutoff of 0.8.

As an example, we show the structural alignment of two IS110 transposases that are distantly related at the level of amino acid percent identity, but have high structural similarity as measured by TM-score (FIG. 14C). These two proteins are 18.1% identical at the amino acid level when aligned using MAFFT G-INS-i algorithm, but have a TM-score of 0.805. This demonstrates that IS110 structures are largely conserved across diverse members, and that the TM-score is a robust metric for detecting these similarities.

When the analysis was restricted to only diverse sequences, TM-scores remained high. We took cluster representatives at 90% amino acid identity and 50% amino acid identity and compared their TM-score distributions (FIG. 14D). We found that the distributions remained roughly the same, suggesting that these TM-scores remain high even when only considering sequences that are highly diverse at the level of primary protein sequence.

We next extracted only the substructures of each protein structure that corresponded with the predicted IS110 Pfam domains. There are many ways that these domains could be defined, and we chose to use the Pfam pHMM domains due to their widespread availability and ease of use. These protein domains were extracted from the tertiary structures using the biopython python package. These extracted domains were then aligned to the IS621 extracted domains using TM-align software (v20220412) as before. Results demonstrate that these protein domain substructures are also highly conserved at TM-score >0.5, with the Tnp domain being especially well conserved (FIG. 14E).

To demonstrate that the TM-score is both sensitive and specific when it comes to identifying IS110 homologs, we compared how the TM-score distribution appeared when aligning the IS621 structure to the structure of IS630 family transposases, which are a completely separate family of transposases that have a transposition mechanism that is completely distinct from that of IS110 family transposases. After downloading these structures and preparing them for analysis, 262 IS630 structures remained. Comparing the TM-score distribution of these protein structures to IS110 structures shows a clear separation between the two (FIG. 14F). All IS630 structures had a TM-score less than 0.50, and 99.7% of IS110 structures had a TM-score above 0.5, demonstrating the utility of the TM-align software and the TM-score metric for identifying structural homologs.

Having determined that these protein structures are highly conserved across diverse primary sequences, we next sought to determine if the distances between highly conserved residues was also conserved within these structures. We identified 5 highly conserved residues in both the RuvC domain and Tnp domains by primary sequence alignment. We extracted these 3-dimensional positions from all of the IS110 AlphaFold structures. For the 5 conserved residues in the RuvC domain, identified as P1-P5, we calculated 3 distances in all structures (FIG. 14G). These distances include D1, which is the distance between P1 and P2; D2, which is taken as the average of the distances between P1 and P3, P1 and P4, and P1 and P5; and D3, which is taken as the average of the distances between P2 and P3, P2 and P4, and P2 and P5. For the 5 conserved residues in the Tnp domain, identified as P1-P5, we calculated 3 distances in all structures. These distances include D1, taken as the average of the distances between P1 and P2, and P1 and P5; D2, taken as the average of the distances between P2 and P3, P2 and P4, P5 and P3, and P5 and P4; and D3, which is the distance between P2 and P5.

Our analysis indicates that the distances between these residues are highly conserved across diverse IS110 protein structures (FIG. 14H). For the RuvC domain, the average D1 distance is 6.57 Å with a standard deviation of 0.809 Å; the average D2 distance is 8.31 Å with a standard deviation of 0.87 Å; and the average D3 distance is 12.2 Å with a standard deviation of 1.01 Å. For the Tnp domain, the average D1 distance is 19.8 Å with a standard deviation of 1.41 Å; the average D2 distance is 23.4 Å with a standard deviation of 0.52 Å; and the average D3 distance is 8.43 Å with a standard deviation of 1.08 Å. Low standard deviations imply that these distances are highly consistent across structures, and that these conserved residues also likely have a conserved structural or enzymatic function.

### Example 16: Assessment of Donor Boundaries of an IS110

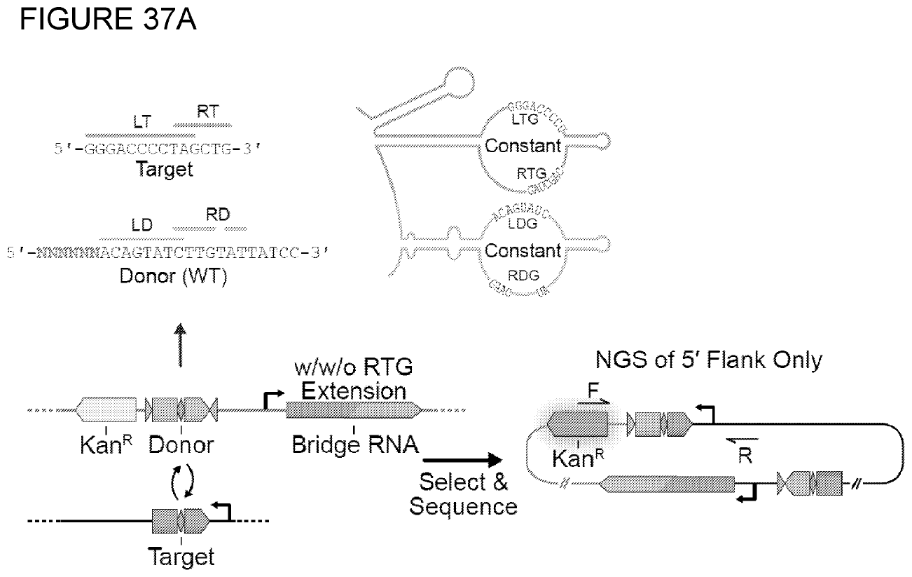

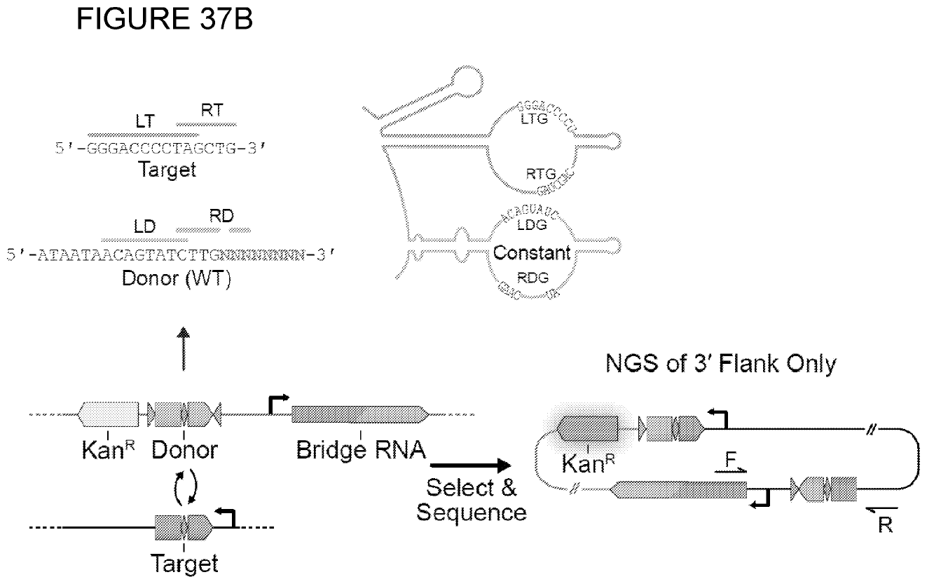

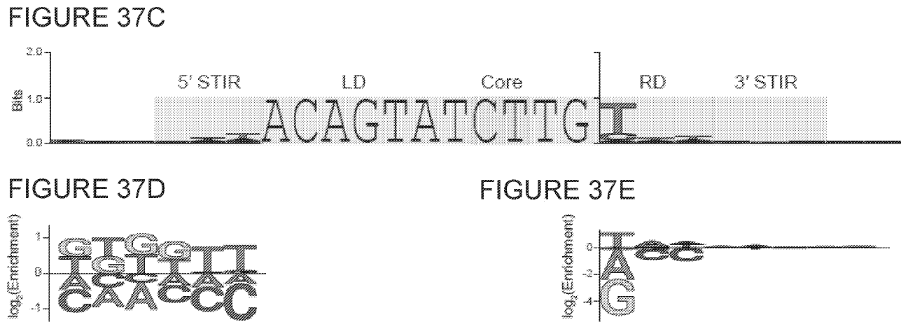

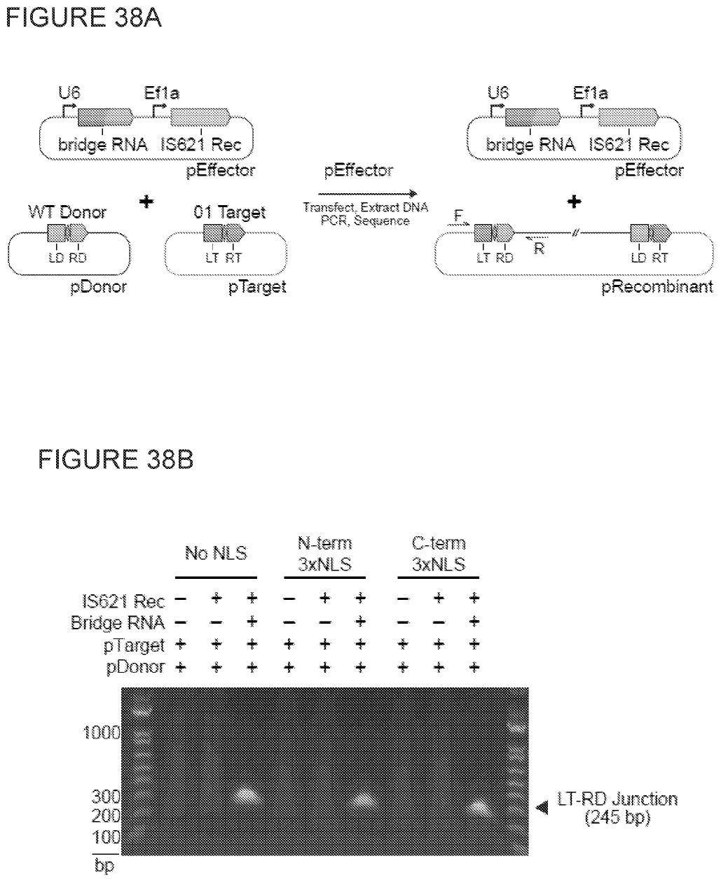

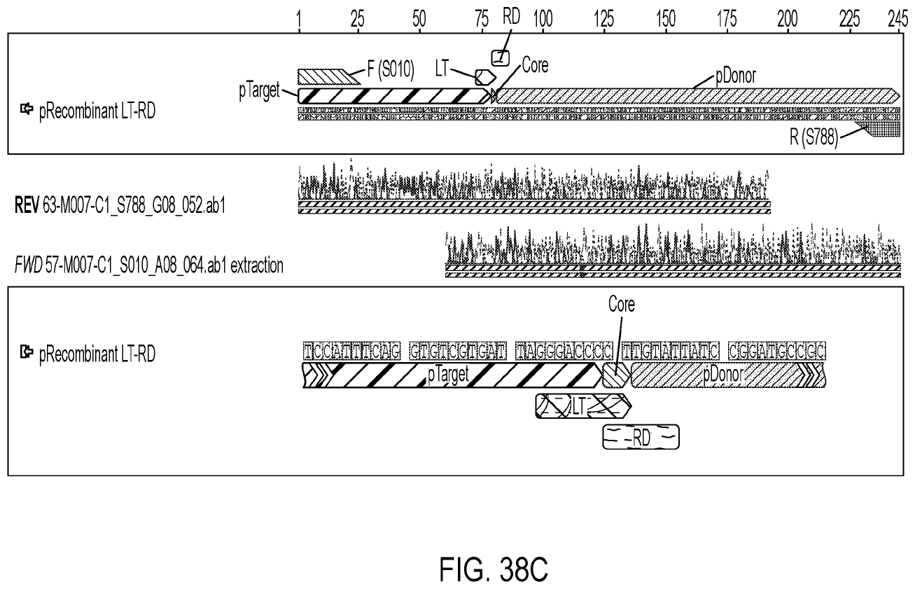

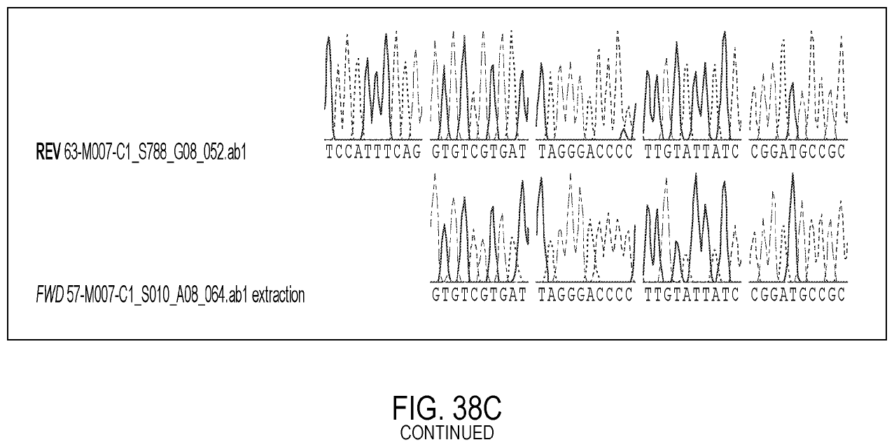

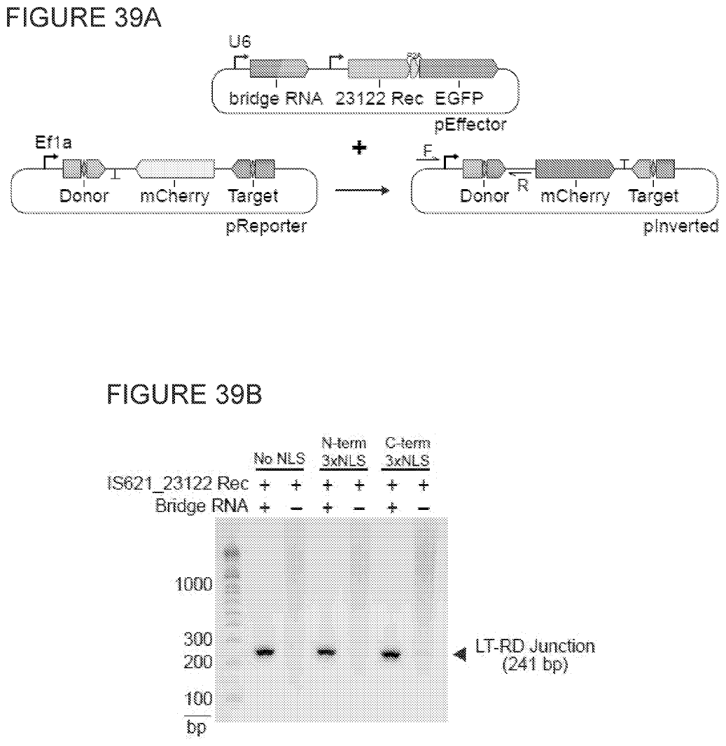

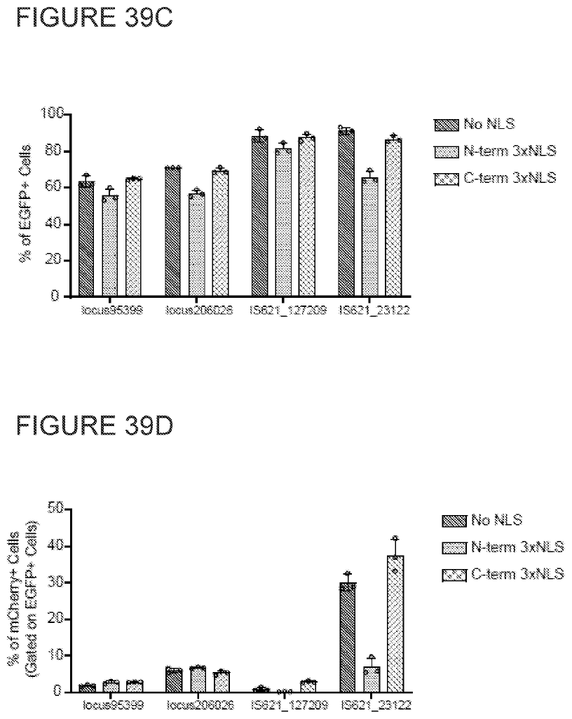

An oligonucleotide encoding the WT donor sequence was purchased with varied N nucleotides incorporated either upstream or downstream of the core sequence as depicted in FIGS. 37A-D. This oligonucleotide was amplified using flanking PCR primers and the resultant library of sequences cloned into a donor backbone which also encodes the bridgeRNA and a kanamycin resistance gene using Golden Gate Assembly. Libraries were then electroporated in Endura DUO electrocompetent cells (Biosearch Technologies). Hundreds of thousands of colonies were isolated for sufficient coverage of the oligo library, and plasmids bearing library members were purified using Nucleobond Xtra Midiprep Kit (Macherey Nagel).

The plasmid libraries encoding thousands of donor boundary sequences were co-electroporated into E. cloni EXPRESS electrocompetent cells (Biosearch Technologies) along with a plasmid encoding the target and the recombinase. A target adjacent promoter results in expression of the kanamycin resistance gene following recombination of the two plasmids, allowing cell survival. After co-electroporation and recovery, cells were plated on bioassay dishes with LB agar. One plating condition, serving as the control, was LB agar with chloramphenicol and ampicillin, which maintain the plasmids but do not induce or require recombination. A second condition was LB agar with chloramphenicol, ampicillin, kanamycin, and 0.07 mM IPTG; IPTG induces recombinase expression, prompting recombination, while kanamycin selects for cells that have induced recombination between the donor and target plasmid. Both conditions were performed in two replicates.

Hundreds of thousands of colonies were scraped from the bioassay dishes and had plasmid DNA extracted using Nucleobond Xtra Midiprep Kit (Macherey Nagel). After plasmid DNA isolation, samples were prepared for next generation sequencing. For DNA isolated from the control conditions, a PCR was used to amplify the barcodes specifying target and bridgeRNA pairs to measure the distribution of barcodes without selecting conditions. For DNA isolated from selection conditions, a PCR was used to amplify the donor-target junction such that the variable donor sequence was captured.

Sequenced amplicons were analyzed using a custom snakemake workflow. First, reads were trimmed using the BBTools package (Bushnell, Rood, and Singer 2017). Reads were aligned to the expected amplicons using BWA-MEM (Li 2013). Only reads that aligned within 5 bp of the expected amplicon were retained. Sequences were extracted from the variable regions on the 5′ and 3′ side of the donor sequence, and the frequency of each sequence was calculated. The preference for each nucleotide at each sequence position was calculated in R, normalizing for the frequency of each nucleotide at each position in the unselected control condition.

### Example 17: Selecting Human Genome Insertion, Inversion, and Excision Candidates

All 14 nt sequences that appeared only one time in the human genome (hg38) were identified using the KMC kmer-counting tool (Kokot, Dlugosz, and Deorowicz 2017). Only sequences with CT or GT cores were retained, resulting in 3,563,893 such sequences. For insertion candidates, sequences were ranked by predicted and observed efficiencies in the target/donor screens. For excision and inversion candidates, all sequences that were within 5 kbp of each other were identified. Pairs of sequences that shared the same core (CT or GT) were retained. These pairs of sequences were then ranked by their predicted efficiencies using the target/donor screen neural net models. Sequences that existed on the same strand were considered to be excision candidates, while sequences on opposite strands were inversion candidates.

### Example 18: Assessing Plasmid-Plasmid Recombination in Human Cells

HEK293T cells were seeded at 18k cells/well in 96-well PDL treated flat bottom plates. Cells were transfected using Lipofectamine 2000 with 100 ng of a 5.8 kb plasmid bearing the recombinase and bridgeRNA (pEffector), 203 ng of 2.9 kb plasmid encoding the donor sequence recognized by the bridgeRNA (pDonor), and 219 ng of a 3.2 kb plasmid encoding the target sequence recognized by the bridgeRNA (pTarget). After 72 hours at 37° C. total DNA was extracted from cells using QuickExtract DNA Extraction Solution (Biosearch Technologies) according to the manufacturer's instructions. Recombination was assessed by performing a PCR across the newly formed LT-RD junction followed by running an agarose gel and performing Sanger sequencing on purified PCR product to verify recombination.

pEffector encodes a bridgeRNA driven by the U6 promoter and the recombinase driven by the Ef1a promoter. In some cases, pEffector lacked a bridgeRNA to serve as a negative control. In some conditions, the recombinase was fused to a N-terminal or C-terminal SV40 NLS repeated 3 times (3×NLS).

### Example 19: Assessing Recombination Activity of Diverse Orthologs in Human Cells

HEK293T cells were seeded at 18k cells/well in 96-well TC-treated flat bottom plates. Cells were transfected using Lipofectamine 2000 with 100 ng of a 5.8 kb plasmid bearing the recombinase and bridgeRNA (pEffector) and 292 ng of a 4.2 kb plasmid encoding an inversion reporter (pReporter). pReporter encodes the Ef1a promoter followed by a donor sequence, an inverted mCherry coding sequence, and a target sequence on the opposite strand of the donor sequence such that recombination between target and donor will result in inversion. The distance between the donor and target in pReporter is 1 kb. pEffector encodes a bridgeRNA driven by the U6 promoter and the recombinase driven by the Ef1a promoter. In some cases, pEffector lacked a bridgeRNA to serve as a negative control. In some conditions, the recombinase was fused to a N-terminal or C-terminal SV40 NLS repeated 3 times (3×NLS). In all cases, the recombinase fusion protein is followed by a P2 Å self-cleaving peptide sequence and a GFP coding sequence.

Efficiency of inversion was measured using flow cytometry. After 72 hours at 37° C., cells were lifted from the plate using TrypLE Express (ThermoFisher), spun down at 200×g for 2 min, refreshed in 100 pL Stain Buffer BSA (BD Pharmingen), and measured using a NovoCyte Quanteon (Agilent). Samples were analyzed by gating for fluorescence against cells transfected with a pEffector lacking a bridgeRNA or encoding an orthogonal effector and lacking a bridgeRNA. The percentage of cells positive for recombination was determined by measuring the percentage of cells expressing EGFP as a proxy for recombinase (and bridgeRNA) expression within the cell. The percentage of cells within the GFP+ population expressing mCherry is reported.

### Example 20: Engineering bridgeRNAs for Increased Efficiency and Specificity

To increase efficiency of bridgeRNAs, additional sequence from the coding sequence of the recombinase found in the natural element was added to the 3′ end of the bridgeRNA determined via either RNA sequencing or computational prediction. Specificity of bridgeRNAs was increased by encoding the LTG to base pair with bases prior to the core only, rather than the bases within the LT and the core.

### Example 21: Delivery and Integration of Large Cargoes into the Human Genome

HEK293T cells were seeded at 18k cells/well in 96-well PDL treated flat bottom plates. Cells were transfected using Lipofectamine 2000 with 137 ng of a 5.8 kb plasmid bearing the recombinase and bridgeRNA and 574 ng of a 4.8 kb plasmid encoding the donor sequence recognized by the bridgeRNA (pDonor). The bridgeRNA target binding loop recognizes a sequence within the human genome. After 72 hours at 37° C. total DNA was extracted from cells using QuickExtract DNA Extraction Solution (Biosearch Technologies) according to the manufacturer's instructions. Recombination was assessed by performing a PCR across the newly formed LT-RD junction followed by running an agarose gel and performing Sanger sequencing on purified PCR product to verify recombination. Off-targets can be assessed using methods known in the art (see e.g., Durrant, Fanton, Tycko NBT 2023).

### Example 22: Inversion of Genomic Loci Using Transient Expression of a Bridge Editor

HEK293T cells were seeded at 18k cells/well in 96-well PDL treated flat bottom plates. Cells were transfected using Lipofectamine 2000 with 600 ng of a 5.8 kb plasmid bearing the recombinase and bridgeRNA. The bridgeRNA was reprogrammed to recognize a donor and target pair within the genome. The intervening sequence length between the recognized target and donor ranges from 0.4-2.6 kb. After 72 hours at 37° C. total DNA was extracted from cells using QuickExtract DNA Extraction Solution (Biosearch Technologies) according to the manufacturer's instructions. Inversion of genomic loci was detected by performing a PCR across the RD-LT and LD-RT junctions, extracting expected PCR products from an agarose gel, and confirming inversion of the locus using Sanger sequencing.

### Example 23: Engineering Split bridgeRNA Systems

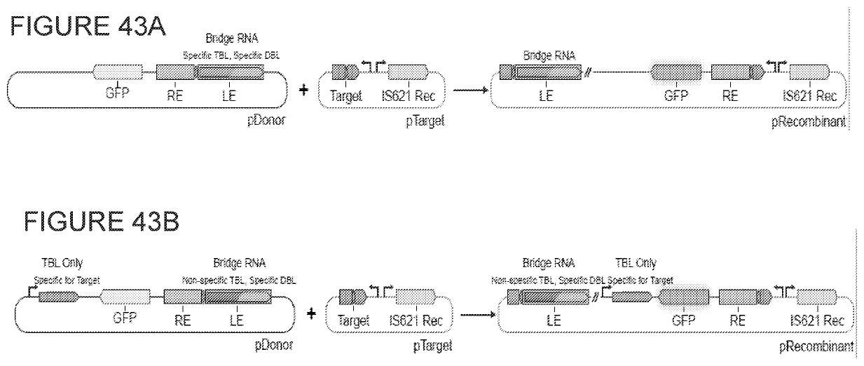

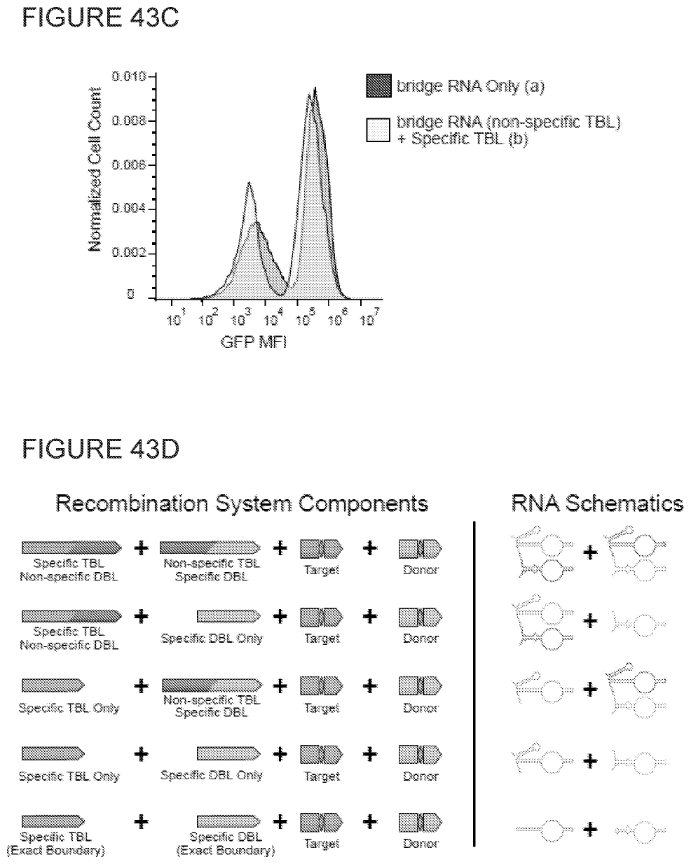

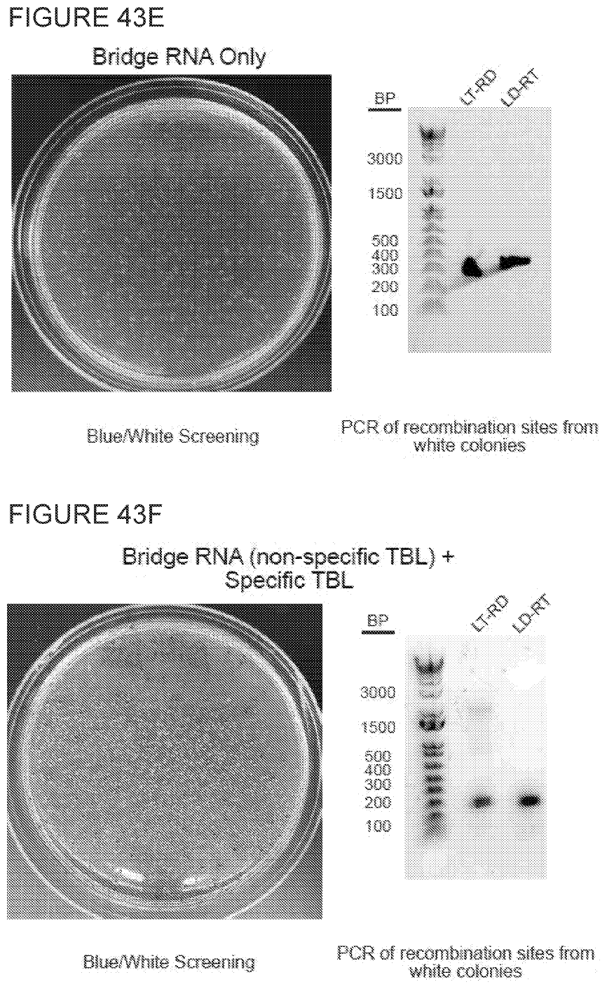

Engineering of a split bridgeRNA system for recombination is shown and described in FIGS. 43A-F.

### Example 24: Mismatch Tolerance

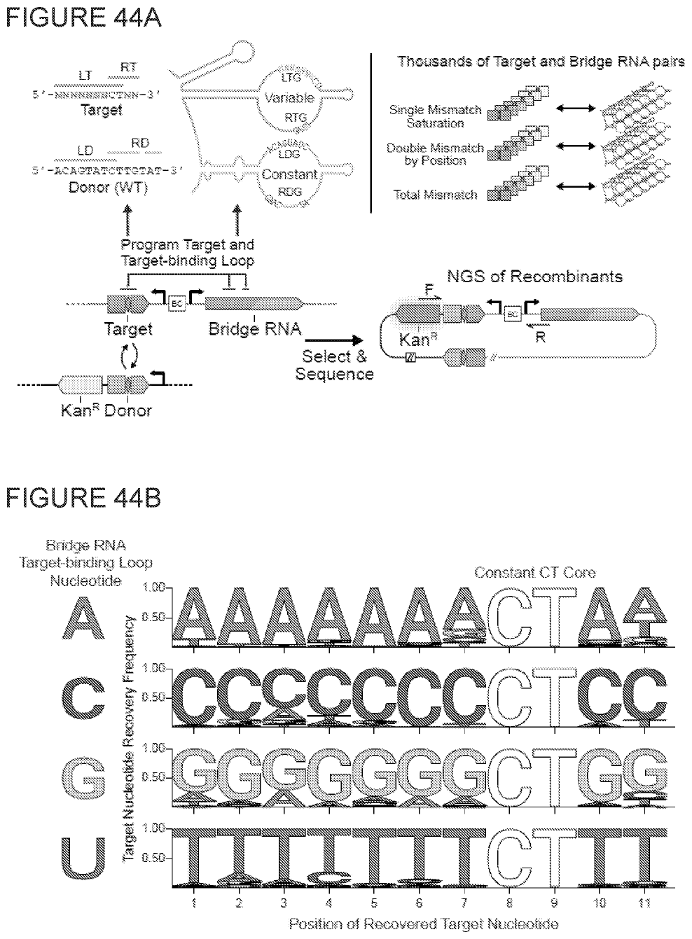

A summary of mismatch tolerance between an IS110 bridgeRNA target binding loop and its target is shown and described in FIGS. 44A-B.

### REFERENCES

U.S. Pat. No. 11,447,770
US20220054239
US20220154224
WO2020191153
WO2021226558
Almeida, Alexandre, Stephen Nayfach, Miguel Boland, Francesco Strozzi, Martin Beracochea, Zhou Jason Shi, Katherine S. Pollard, et al. 2021. “A Unified Catalog of 204,938 Reference Genomes from the Human Gut Microbiome.” Nature Biotechnology 39 (1): 105-14.
Alonso-Lerma, Borja, Ylenia Jabalera, Matias Morin, Almudena Fernandez, Sara Samperio, Ane Quesada, Antonio Reifs, et al. 2022. “Evolution of CRISPR-Associated Endonucleases as Inferred from Resurrected Proteins.” bioRxiv. doi.org/10.1101/2022.03.30.485982.
Altae-Tran, Han, Soumya Kannan, F. Esra Demircioglu, Rachel Oshiro, Suchita P. Nety, Luke J. McKay, Mensur Dlakid, et al. 2021. “The Widespread IS200/IS605 Transposon Family Encodes Diverse Programmable RNA-Guided Endonucleases.” Science 374 (6563): 57-65.
Altschul, S. F., W. Gish, W. Miller, E. W. Myers, and D. J. Lipman. 1990. “Basic Local Alignment Search Tool.” Journal of Molecular Biology 215 (3): 403-10.
Anderson, Rachelle P., Eugenia Voziyanova, and Yuri Voziyanov. 2012. “Flp and Cre Expressed from Flp-2A-Cre and Flp-IRES-Cre Transcription Units Mediate the Highest Level of Dual Recombinase-Mediated Cassette Exchange.” Nucleic Acids Research 40 (8): e62.
Anzalone, Andrew V., Xin D. Gao, Christopher J. Podracky, Andrew T. Nelson, Luke W. Koblan, Aditya Raguram, Jonathan M. Levy, Jaron A. M. Mercer, and David R. Liu. 2021. “Programmable Deletion, Replacement, Integration and Inversion of Large DNA Sequences with Twin Prime Editing.” Nature Biotechnology 40 (5): 731-40.
Anzalone, Andrew V., Peyton B. Randolph, Jessie R. Davis, Alexander A. Sousa, Luke W. Koblan, Jonathan M. Levy, Peter J. Chen, et al. 2019. “Search-and-Replace Genome Editing without Double-Strand Breaks or Donor DNA.” Nature 576 (7785): 149-57.
Anzalone, A. V., Koblan, L. W. & Liu, D. R. Genome editing with CRISPR-Cas nucleases, base editors, transposases and prime editors. Nat Biotechnol 38, 824-844 (2020).
Araki, K., M. Araki, and K. Yamamura. 1997. “Targeted Integration of DNA Using Mutant Lox Sites in Embryonic Stem Cells.” Nucleic Acids Research 25 (4): 868-72.
Berrios, K. N., Evitt, N. H., DeWeerd, R. A. et al. Controllable genome editing with split-engineered base editors. Nat Chem Biol 17, 1262-1270 (2021).
Breuer R., Gomes-Filho, J-V., Randau, L., “Conservation of Archaeal C/D Box sRNA-Guided RNA modifications.” Frontiers in Microbiology (12): (2021).
Bushnell, Brian, Jonathan Rood, and Esther Singer. 2017. “BBMerge -Accurate Paired Shotgun Read Merging via Overlap.” PloS One 12 (10): e0185056.
Camarillo-Guerrero, Luis F., Alexandre Almeida, Guillermo Rangel-Pineros, Robert D. Finn, and Trevor D. Lawley. 2021. “Massive Expansion of Human Gut Bacteriophage Diversity.” Cell 184 (4): 1098-1109.e9.
Chandler, Michael, Olivier Fayet, Philippe Rousseau, Bao Ton Hoang, and Guy Duval-Valentin. 2015. “Copy-out-Paste-in Transposition of IS911: A Major Transposition Pathway.” Microbiology Spectrum 3 (4). doi.org/10.1128/microbiolspec.MDNA3-0031-2014.
Chen, I-Min A., Ken Chu, Krishnaveni Palaniappan, Anna Ratner, Jinghua Huang, Marcel Huntemann, Patrick Hajek, et al. 2021. “The IMG/M Data Management and Analysis System v.6.0: New Tools and Advanced Capabilities.” Nucleic Acids Research 49 (D1): D751-63.
Chen, L., Park, J. E., Paa, P. et al. Programmable C:G to G:C genome editing with CRISPR-Cas9-directed base excision repair proteins. Nat Commun 12, 1384 (2021).
Choi, Sunju, Shinya Ohta, and Eiichi Ohtsubo. 2003. “A Novel IS Element, IS621, of the IS110/IS492 Family Transposes to a Specific Site in Repetitive Extragenic Palindromic Sequences in Escherichia Coli.” Journal of Bacteriology 185 (16): 4891-4900.
Chollet, Frangois, and Others. 2015. “Keras.” 2015. keras.io
Durrant, Matthew G., Alison Fanton, Josh Tycko, Michaela Hinks, Sita S. Chandrasekaran, Nicholas T. Perry, Julia Schaepe. 2022. “Systematic Discovery of Recombinases for Efficient Integration of Large DNA Sequences into the Human Genome.” Nature Biotechnology, October. doi.org/10.1038/s41587-022-01494-w.
Durrant, Matthew G., Michelle M. Li, Benjamin A. Siranosian, Stephen B. Montgomery, and Ami S. Bhatt. 2020. “A Bioinformatic Analysis of Integrative Mobile Genetic Elements Highlights Their Role in Bacterial Adaptation.” Cell Host & Microbe 28 (5): 767.
Edgar, Robert C. 2004. “MUSCLE: A Multiple Sequence Alignment Method with Reduced Time and Space Complexity.” BMC Bioinformatics 5 (August): 113.
Ekeberg, Magnus, Cecilia Lovkvist, Yueheng Lan, Martin Weigt, and Erik Aurell. 2013. “Improved Contact Prediction in Proteins: Using Pseudolikelihoods to Infer Potts Models.” Physical Review. E, Statistical, Nonlinear, and Soft Matter Physics 87 (1): 012707.
Farruggio, A. P., & Calos, M. P. (2014). Serine integrase chimeras with activity in E. coli and HeLa cells. Biology open, 3(10), 895-903.
Finn, Robert D., Jody Clements, and Sean R. Eddy. 2011. “HMMER Web Server: Interactive Sequence Similarity Searching.” Nucleic Acids Research 39 (Web Server issue): W29-37.
Forster, Samuel C., Nitin Kumar, Blessing O. Anonye, Alexandre Almeida, Elisa Viciani, Mark D. Stares, Matthew Dunn, et al. 2019. “A Human Gut Bacterial Genome and Culture Collection for Improved Metagenomic Analyses.” Nature Biotechnology 37 (2): 186-92.
Gaudelli, Nicole M., Alexis C. Komor, Holly A. Rees, Michael S. Packer, Ahmed H. Badran, David I. Bryson, and David R. Liu. 2017. “Programmable Base Editing of A⋅T to G⋅C in Genomic DNA without DNA Cleavage.” Nature 551 (7681): 464-71.
Gómez-Garcia, Guillermo, Angel Ruiz-Enamorado, Luis Yuste, Fernando Rojo, and Renata Moreno. 2021. “Expression of the ISPpu9 Transposase of Pseudomonas Putida KT2440 Is Regulated by Two Small RNAs and the Secondary Structure of the mRNA 5′-Untranslated Region.” Nucleic Acids Research 49 (16): 9211-28.
Grunewald, Julian, Bret R. Miller, Regan N. Szalay, Peter K. Cabeceiras, Christopher J. Woodilla, Eliza Jane B. Holtz, Karl Petri, and J. Keith Joung. 2022. “Engineered CRISPR Prime Editors with Compact, Untethered Reverse Transcriptases.” Nature Biotechnology, September. doi.org/10.1038/s41587-022-01473-1.
Higgins, Brian P., Chandra D. Carpenter, and Anna C. Karls. 2007. “Chromosomal context directs high-frequency precise excision of IS492 in Pseudoalteromonas atlantica.” Proceedings of the National Academy of Sciences 104 (6): 1901-6.
Higgins, Brian P., Adam C. Popkowski, Peter R. Caruana, and Anna C. Karls. 2009. “Site-Specific Insertion of IS 492 in Pseudoalteromonas atlantica.” Journal of Bacteriology 191 (20): 6408-14.
Huang, Liang, He Zhang, Dezhong Deng, Kai Zhao, Kaibo Liu, David A. Hendrix, and David H. Mathews. 2019. “LinearFold: Linear-Time Approximate RNA Folding by 5′-to-3′ Dynamic Programming and Beam Search.” Bioinformatics 35 (14): i295-304.
Hyatt, Doug, Gwo-Liang Chen, Philip F. Locascio, Miriam L. Land, Frank W. Larimer, and Loren J. Hauser. 2010. “Prodigal: Prokaryotic Gene Recognition and Translation Initiation Site Identification.” BMC Bioinformatics 11 (March): 119.
Ioannidi, Eleonora I., Matthew T. N. Yarnall, Cian Schmitt-Ulms, Rohan N. Krajeski, Justin Lim, Lukas Villiger, Wenyuan Zhou, et al. 2021. “Drag-and-Drop Genome Insertion without DNA Cleavage with CRISPR-Directed Integrases.” bioRxiv. doi.org/10.1101/2021.11.01.466786.
Karvelis, Tautvydas, Gytis Druteika, Greta Bigelyte, Karolina Budre, Rimante Zedaveinyte, Arunas Silanskas, Darius Kazlauskas, Ceslovas Venclovas, and Virginijus Siksnys. 2021. “Transposon-Associated TnpB Is a Programmable RNA-Guided DNA Endonuclease.” Nature 599 (7886): 692-96.
Katoh, Kazutaka, Kazuharu Misawa, Kei-Ichi Kuma, and Takashi Miyata. 2002. “MAFFT: A Novel Method for Rapid Multiple Sequence Alignment Based on Fast Fourier Transform.” Nucleic Acids Research 30 (14): 3059-66.
Kempen, Michel van, Stephanie S. Kim, Charlotte Tumescheit, Milot Mirdita, Jeongjae Lee, Cameron L. M. Gilchrist, Johannes Soding, and Martin Steinegger. 2023. “Fast and Accurate Protein Structure Search with Foldseek.” Nature Biotechnology, May. doi.org/10.1038/s41587-023-01773-0.
Klompe, Sanne E., Phuc L. H. Vo, Tyler S. Halpin-Healy, and Samuel H. Sternberg. 2019. “Transposon-Encoded CRISPR-Cas Systems Direct RNA-Guided DNA Integration.” Nature 571 (7764): 219-25.
Kokot, Marek, Maciej Dlugosz, and Sebastian Deorowicz. 2017. “KMC 3: Counting and Manipulating K-Mer Statistics.” Bioinformatics 33 (17): 2759-61.
Komor, Alexis C., Yongjoo B. Kim, Michael S. Packer, John A. Zuris, and David R. Liu. 2016. “Programmable Editing of a Target Base in Genomic DNA without Double-Stranded DNA Cleavage.” Nature 533 (7603): 420-24.
Lebar, Tina, Dusko Lainscek, Estera Merljak, Jana Aupic, and Roman Jerala. 2020. “A Tunable Orthogonal Coiled-Coil Interaction Toolbox for Engineering Mammalian Cells.” Nature Chemical Biology 16 (5): 513-19.
Li, Heng. 2018. “Minimap2: Pairwise Alignment for Nucleotide Sequences.” Bioinformatics 34 (18): 3094-3100.
Li, Heng, and Richard Durbin. 2009. “Fast and Accurate Short Read Alignment with Burrows-Wheeler Transform.” Bioinformatics 25 (14): 1754-60.
Li, Heng. 2013. “Aligning Sequence Reads, Clone Sequences and Assembly Contigs with BWA-MEM.” arXiv [q-bio.GN]. arXiv. arxiv.org/abs/1303.3997.
Makarova, Kira S., Yuri I. Wolf, Jaime Iranzo, Sergey A. Shmakov, Omer S. Alkhnbashi, Stan J. J. Brouns, Emmanuelle Charpentier, et al. 2020. “Evolutionary Classification of CRISPR-Cas Systems: A Burst of Class 2 and Derived Variants.” Nature Reviews. Microbiology 18 (2): 67-83.
Meyer, F., D. Paarmann, M. D'Souza, R. Olson, E. M. Glass, M. Kubal, T. Paczian, et al. 2008. “The Metagenomics RAST Server—a Public Resource for the Automatic Phylogenetic and Functional Analysis of Metagenomes.” BMC Bioinformatics 9 (September): 386.
Minh, Bui Quang, Heiko A. Schmidt, Olga Chernomor, Dominik Schrempf, Michael D. Woodhams, Arndt von Haeseler, and Robert Lanfear. 2020. “IQ-TREE 2: New Models and Efficient Methods for Phylogenetic Inference in the Genomic Era.” Molecular Biology and Evolution 37 (5): 1530-34.
Mitchell, Alex L., Alexandre Almeida, Martin Beracochea, Miguel Boland, Josephine Burgin, Guy Cochrane, Michael R. Crusoe, et al. 2020. “MGnify: The Microbiome Analysis Resource in 2020.” Nucleic Acids Research 48 (D1): D570-78.
Nakamura, Muneaki, Yuchen Gao, Antonia A. Dominguez, and Lei S. Qi. 2021. “CRISPR Technologies for Precise Epigenome Editing.” Nature Cell Biology 23 (1): 11-22.
Nawrocki, Eric P., and Sean R. Eddy. 2013. “Infernal 1.1: 100-Fold Faster RNA Homology Searches.” Bioinformatics 29 (22): 2933-35.
O'Malley, Tom, Elie Bursztein, James Long, Frangois Chollet, Haifeng Jin, Luca Invernizzi, and Others. 2019. “KerasTuner.” 2019. github.com/keras-team/keras-tuner.
Pallarès-Masmitjà, Maria, Dimitrije Ivancid, Jnlia Mir-Pedrol, Jessica Jaraba-Wallace, Tommaso Tagliani, Baldomero Oliva, Amal Rahmeh, Avencia Sanchez-Mejias, and Marc Guell. 2021. “Find and Cut-and-Transfer (FiCAT) Mammalian Genome Engineering.” Nature Communications 12 (1): 1-9.
Paradis, Emmanuel, and Klaus Schliep. 2019. “Ape 5.0: An Environment for Modem Phylogenetics and Evolutionary Analyses in R.” Bioinformatics 35 (3): 526-28.
Partridge, Sally R., and Ruth M. Hall. 2003. “The IS1111 Family Members IS4321 and IS5075 Have Subterminal Inverted Repeats and Target the Terminal Inverted Repeats of Tn21 Family Transposons.” Journal of Bacteriology 185 (21): 6371-84.
Perkins-Balding, Donna, Guy Duval-Valentin, and Anna C. Glasgow. 1999. “Excision of IS492 Requires Flanking Target Sequences and Results in Circle Formation in Pseudoalteromonas atlantica.” Journal of Bacteriology 181 (16): 4937-48.
Quinlan, Aaron R., and Ira M. Hall. 2010. “BEDTools: A Flexible Suite of Utilities for Comparing Genomic Features.” Bioinformatics 26 (6): 841-42.
Revell, Liam J. 2012. “Phytools: An R Package for Phylogenetic Comparative Biology (and Other Things).” Methods in Ecology and Evolution. doi.org/10.1111/j.2041-210x.2011.00169.x.
Ross, Karen, Alessandro M. Varani, Erik Snesrud, Hongzhan Huang, Danillo Oliveira Alvarenga, Jian Zhang, Cathy Wu, Patrick McGann, and Mick Chandler. 2021.
“TnCentral: A Prokaryotic Transposable Element Database and Web Portal for Transposon Analysis.” mBio 12 (5): e0206021.
Rubin, Benjamin E., Spencer Diamond, Brady F. Cress, Alexander Crits-Christoph, Yue Clare Lou, Adair L. Borges, Haridha Shivram, et al. 2022. “Species- and Site-Specific Genome Editing in Complex Bacterial Communities.” Nature Microbiology 7 (1): 34-47.
Siguier, P., E. Gourbeyre, A. Varani, B. Ton-Hoang, and M. Chandler. 2015. “Everyman's Guide to Bacterial Insertion Sequences. Microbiol Spectr 3.” MDNA3-0030-2014.
Siguier, P., J. Perochon, L. Lestrade, J. Mahillon, and M. Chandler. 2006. “ISfinder: The Reference Centre for Bacterial Insertion Sequences.” Nucleic Acids Research 34 (Database issue): D32-36.
Steinegger, Martin, and Johannes Soding. 2017. “MMseqs2 Enables Sensitive Protein Sequence Searching for the Analysis of Massive Data Sets.” Nature Biotechnology 35 (11): 1026-28.
Strecker, Jonathan, Alim Ladha, Zachary Gardner, Jonathan L. Schmid-Burgk, Kira S. Makarova, Eugene V. Koonin, and Feng Zhang. 2019. “RNA-Guided DNA Insertion with CRISPR-Associated Transposases.” Science 365 (6448): 48-53.
Sunagawa, Shinichi, Luis Pedro Coelho, Samuel Chaffron, Jens Roat Kultima, Karine Labadie, Guillem Salazar, Bardya Djahanschiri, et al. 2015. “Ocean Plankton. Structure and Function of the Global Ocean Microbiome.” Science 348 (6237): 1261359.
Tagashira, Masaki, and Kiyoshi Asai. 2022. “ConsAlifold: Considering RNA Structural Alignments Improves Prediction Accuracy of RNA Consensus Secondary Structures.” Bioinformatics 38 (3): 710-19.
Tak, Y. Esther, Benjamin P. Kleinstiver, James K. Nunez, Jonathan Y. Hsu, Joy E. Horng, Jingyi Gong, Jonathan S. Weissman, and J. Keith Joung. 2017. “Inducible and Multiplex Gene Regulation Using CRISPR-Cpf1-Based Transcription Factors.” Nature Methods 14 (12): 1163-66.
“The CRISPR/Cas System: Emerging Technology and Application.” n.d. Accessed Nov. 1, 2022. www.caister.com/crispr.
Thomson, James G., Edmund B. Rucker 3rd, and Jorge A. Piedrahita. 2003. “Mutational Analysis of loxP Sites for Efficient Cre-Mediated Insertion into Genomic DNA.” Genesis 36 (3): 162-67.
Ton-Hoang, B., M. Betermier, P. Polard, and M. Chandler. 1997. “Assembly of a Strong Promoter Following IS911 Circularization and the Role of Circles in Transposition.” The EMBO Journal 16 (11): 3357-71.
Tou, C. J., Klienstiver, B. P., Recent advances in double-strand break-free kilobase-scale genome editing technologies. Biochem (2022).
Vo, P. L. H., Ronda, C., Klompe, S. E. et al. CRISPR RNA-guided integrases for high-efficiency, multiplexed bacterial genome engineering. Nat Biotechnol 39, 480-489 (2021).
Wei, Jingyi, Peter Lotfy, Kian Faizi, Eleanor Wang, Hannah Slabodkin, Emily Kinnaman, Sita Chandrasekaran, et al. n.d. “Deep Learning and CRISPR-Cas13d Ortholog Discovery for Optimized RNA Targeting.” doi.org/10.1101/2021.09.14.460134.
Weinberg, Zasha, and Ronald R. Breaker. 2011. “R2R—Software to Speed the Depiction of Aesthetic Consensus RNA Secondary Structures.” BMC Bioinformatics 12 (January): 3.
Xu, X., Chemparathy, A., Zeng, L., Shang, S., Nakamura, M., et al., “Engineered miniature CRISPR-Cas system for mammalian genome regulation and editing.” Mol Cell 81(20): 4333-4345. (2021).
Yang, Lei, Alec A. K. Nielsen, Jesus Fernandez-Rodriguez, Conor J. McClune, Michael T. Laub, Timothy K. Lu, and Christopher A. Voigt. 2014. “Permanent Genetic Memory with >1-Byte Capacity.” Nature Methods 11 (12): 1261-66.
Youngblut, Nicholas D., Jacobo de la Cuesta-Zuluaga, Georg H. Reischer, Silke Dauser, Nathalie Schuster, Chris Walzer, Gabrielle Stalder, Andreas H. Farnleitner, and Ruth E. Ley. 2020. “Large-Scale Metagenome Assembly Reveals Novel Animal-Associated Microbial Genomes, Biosynthetic Gene Clusters, and Other Genetic Diversity.” mSystems 5 (6). doi.org/10.1128/mSystems.01045-20.
Yu G, Zhao Y, Li H. The multistructural forms of box C/D ribonucleoprotein particles. RNA. 2018 December; 24(12):1625-1633. doi: 10.1261/rna.068312.118. Epub 2018 Sep. 25
Yu, Guangchuang. 2020. “Using Ggtree to Visualize Data on Tree-Like Structures.” Current Protocols in Bioinformatics/Editoral Board, Andreas D. Baxevanis . . . [et Al.]69 (1): e96.
Zhai, Haotian, Li Cui, Zhen Xiong, Qingsheng Qi, and Jin Hou. 2022. “CRISPR—Mediated Protein-Tagging Signal Amplification Systems for Efficient Transcriptional Activation and Repression in Saccharomyces Cerevisiae.” Nucleic Acids Research 50 (10): 5988-6000
Zhang, Yang, and Jeffrey Skolnick. 2004. “Scoring Function for Automated Assessment of Protein Structure Template Quality.” Proteins 57 (4): 702-10.
Zhang, Yang, and Jeffrey Skolnick. 2005. “TM-Align: A Protein Structure Alignment Algorithm Based on the TM-Score.” Nucleic Acids Research 33 (7): 2302-9.

What is claimed is:

1. A recombinant nucleic acid editing system comprising:
a) an IS110 family transposase, or a nucleic acid comprising a sequence encoding the IS110 family transposase; and
b) a nucleic acid comprising a sequence encoding a bridgeRNA.

2. The recombinant nucleic acid editing system of claim 1, wherein the nucleic acid comprising a sequence encoding a bridgeRNA comprises a left end (LE) sequence of a transposon that encodes the IS110 family transposase.

3. The recombinant nucleic acid editing system of claim 1, wherein the nucleic acid comprising a sequence encoding a bridgeRNA comprises a right end (RE) sequence of a transposon that encodes the IS110 family transposase.

4. The recombinant nucleic acid editing system of claims 1-3, further comprising a nucleic acid comprising a RE sequence and a LE sequence or a RE sequence, a core sequence, and a LE sequence of the IS110 element that encodes the IS110 family transposase.

5. The recombinant nucleic acid editing system of claims 1-3, further comprising a nucleic acid comprising a right flank (RF) sequence and a left flank (LF) sequence or a RF sequence, a core sequence, and a LF sequence of the target site sequence for the IS110 family transposase.

6. The recombinant nucleic acid editing system of claim 4, wherein the nucleic acid comprising the RE sequence and the LE sequence or the RE sequence, the core sequence, and the LE sequence further comprises a nucleic acid sequence for insertion into a target site sequence.

7. The recombinant nucleic acid editing system of claim 6, wherein the target site sequence comprises a RF sequence and a LF sequence or a RF sequence, a core sequence, and a LF sequence for the IS110 family transposase.

8. The recombinant nucleic acid editing system of claim 5, wherein the nucleic acid comprising the RF sequence and the LF sequence or the RF sequence, the core sequence, and the LF sequence further comprises a nucleic acid sequence for insertion into a donor site sequence.

9. The recombinant nucleic acid editing system of claim 8, wherein the donor site sequence comprises a RE sequence and a LE sequence or a RE sequence, a core sequence, and a LE sequence of the IS110 element that encodes the IS110 family transposase.

10. The recombinant nucleic acid editing system of claims 1-9, wherein the bridgeRNA comprises a nucleotide sequence at least 50% identical to a bridgeRNA sequence of SEQ ID NOS: 1-348 or SEQ ID NOS: 349-10175.

11. The recombinant nucleic acid editing system of claims 1-9, wherein said RE sequence, comprises a RE sequence of SEQ ID NOS: 1-348, 30354-30529, 349-10175 or 30530-40356, said LE sequence comprises a LE sequence of SEQ ID NOS: 1-348, 30354-30529, 349-10175 or 30530-40356, and/or said core sequence comprises a core sequence of SEQ ID NOS: 1-348, 30354-30529, 349-10175 or 30530-40356.

12. A recombinant nucleic acid editing system comprising:
a) an IS110 family transposase, or a nucleic acid comprising a sequence encoding the IS110 family transposase; and
b) a bridgeRNA, or a nucleic acid comprising a sequence encoding the bridgeRNA, the bridgeRNA comprising at least one stem-loop structure and further comprising at least one internal loop comprising a first nucleotide sequence that is complementary to a first target site sequence of a target DNA, a second nucleotide sequence that is complementary to a second target site sequence which is on the opposite strand of the target DNA to the first target site sequence and wherein the bridgeRNA is capable of forming a complex with the IS110 family transposase.

13. The recombinant nucleic acid editing system of claim 12, wherein the bridgeRNA further comprises a third nucleotide sequence that is complementary to a first donor site sequence of a donor DNA, and a fourth nucleotide sequence that is complementary to a second donor site sequence which is on the opposite strand of the donor DNA to the first donor site sequence.

14. The recombinant nucleic acid editing system of claim 13, wherein the third nucleotide sequence and the fourth nucleotide sequence are on a second internal loop.

15. The recombinant nucleic acid editing system of claims 1-14, wherein the IS110 family transposase comprises a RuvC-like DEDD catalytic domain and a transposase domain.

16. The recombinant nucleic acid editing system of claim 15, wherein the IS110 family transposase further comprises a linker domain between the RuvC-like DEDD catalytic domain and transposase domain.

17. The recombinant nucleic acid editing system of claim 16, wherein the linker domain comprises a coiled-coil linker domain.

18. The recombinant nucleic acid editing system of claims 15-17, wherein the RuvC-like DEDD catalytic domain comprises an amino acid sequence at least 50% identical to a RuvC-like DEDD catalytic domain sequence of SEQ ID NOs: 10176-10523, 10524-20350, or 40357-516430.

19. The recombinant nucleic acid editing system of claims 15-17, wherein the IS110 family transposase comprises a RuvC-like DEDD catalytic domain that forms a similar tertiary structure to the RuvC-like DEDD catalytic domain of IS621.

20. The recombinant nucleic acid editing system of claim 19, wherein the IS110 family transposase RuvC-like DEDD catalytic domain comprises a tertiary structure similar to a tertiary structure of the RuvC-like DEDD catalytic domain of IS621 if the template modeling score (TM-score) for the RuvC-like DEDD catalytic domain of the IS110 family transposase is 0.5 or higher.

21. The recombinant nucleic acid editing system of claim 20, wherein the RuvC-like DEDD catalytic domain comprises an amino acid sequence at least 15% identical to a RuvC-like DEDD catalytic domain sequence of SEQ ID NOs: 10176-10523, 10524-20350, or 40357-516430.

22. The recombinant nucleic acid editing system of claims 15-17, wherein the transposase domain comprises an amino acid sequence at least 50% identical to a transposase domain sequence of SEQ ID NOs: 10176-10523, 10524-20350, or 40357-516430.

23. The recombinant nucleic acid editing system of claims 15-17, wherein the IS110 family transposase comprises a transposase domain that forms a similar tertiary structure to the transposase domain of IS621.

24. The recombinant nucleic acid editing system of claim 23, wherein the IS110 family transposase transposase domain comprises a tertiary structure similar to a tertiary structure of the transposase domain of IS621 if the template modeling score (TM-score) for the transposase domain of the IS110 family transposase is 0.5 or higher.

25. The recombinant nucleic acid editing system of claim 24, wherein the transposase domain comprises an amino acid sequence at least 15% identical to a transposase domain sequence of SEQ ID NOs: 10176-10523, 10524-20350, or 40357-516430.

26. The recombinant nucleic acid editing system of claims 15-17, wherein the IS110 family transposase comprises an amino acid sequence at least 50% identical to SEQ ID NOs: 10176-10523, 10524-20350, or 40357-516430.

27. The recombinant nucleic acid editing system of claims 15-17, wherein the IS110 family transposase further comprises a tertiary structure similar to a tertiary structure of IS621.

28. The recombinant nucleic acid editing system of claim 27, wherein the IS110 family transposase comprises a tertiary structure similar to a tertiary structure of IS621 if the template modeling score (TM-score) for the transposase is 0.5 or higher.

29. The recombinant nucleic acid editing system of claim 28, wherein the transposase domain comprises an amino acid sequence at least 15% identical to a transposase domain sequence of SEQ ID NOs: 10176-10523, 10524-20350, or 40357-516430.

30. The recombinant nucleic acid editing system of claims 1-29, wherein the IS110 family transposase is an IS110 group transposase.

31. The recombinant nucleic acid editing system of claims 1-29, wherein the IS110 family transposase is an IS1111 group transposase.

32. The recombinant nucleic acid editing system of claim 31, wherein the IS1111 group transposase is IS111IA or IS1111_229727.

33. The recombinant nucleic acid editing system of claim 30, wherein the IS110 group transposase is IS621, ISPa11, IsPa29, ISMmg1, ISPfl1, ISMae40, ISStma6, ISAzs32, ISMex9, ISCARN28, ISAarl6, ISCps7, ISPpu9, ISRel9, ISEsa2, ISMma5, IS900, or ISHne5.

34. The recombinant nucleic acid editing system of claim 30, wherein the IS110 group transposase comprises an amino acid sequence at least 50% identical to IS621 (SEQ ID NO: 10176).

35. The recombinant nucleic acid editing system of claims 12-34, wherein the bridgeRNA comprises at least two stem-loop structures comprising a first stem-loop and a second stem-loop where the first stem-loop is 5′ to the second stem-loop and wherein the first stem-loop comprises a target binding loop and the second stem-loop comprises a donor binding loop.

36. The recombinant nucleic acid editing system of claim 35, wherein the bridgeRNA further comprises a third stem-loop structure 5′ of the first stem-loop.

37. The recombinant nucleic acid editing system of claims 35-36, wherein the stem of the first stem-loop is 5 to 35 nucleotides long and the loop is 3-10 nucleotides long, the target binding loop is 5 to 20 nucleotides long, the stem of the second stem-loop is 5 to 35 nucleotides long and the loop is 3-10 nucleotides long, and the donor binding loop is 5 to 20 nucleotides long.

38. The recombinant nucleic acid editing system of claim 37, wherein the stem of the second stem-loop structure comprises 1 to 4 loops or bubbles that are each 1 to 10 nucleotides long.

39. The recombinant nucleic acid editing system of claims 12-34, wherein the bridgeRNA comprises a nucleotide sequence comprising any of the 5′ to 3′ sequences provided in FIG. 19, wherein “n” represents any nucleotide, “R” represents an A or G nucleotide, and “Y” represents a C or U nucleotide.

40. The recombinant nucleic acid editing system of claim 39, wherein the bridgeRNA comprises a 5′ to 3′ secondary structure provided in the first row, second row, third row, or fourth row of secondary structure for said sequence provided in FIG. 19, wherein matching parentheses “(“and”)” indicate base-paired nucleotides, and “.” indicate unpaired bases.

41. The recombinant nucleic acid editing system of claims 12-34, wherein the bridgeRNA comprises a stem-loop structure as depicted in FIG. 2D, FIG. 11B, or FIG. 13.

42. The recombinant nucleic acid editing system of claims 12-41, wherein the target binding loop of the bridgeRNA comprises:
a left-target guide (LTG) comprising, in the 5′ to 3′ direction, a nucleotide sequence complementary to first strand of a target site sequence wherein the 3′ end of the LTG is complementary to at least one of the nucleotides of a core sequence on the first strand of the target site sequence; and
a right-target guide (RTG) comprising, in the 5′ to 3′ direction, a nucleotide sequence that is a reverse complement to an opposite strand of the first strand of the target site sequence wherein the 3′ end of the RTG is reverse complementary to at least one of the nucleotides of the core sequence on the opposite strand of the first strand of the target site sequence;
wherein the target site sequence is a polynucleotide sequence; and/or
wherein the donor binding loop of the bridgeRNA comprises:
a left-donor guide (LDG) comprising, in the 5′ to 3′ direction, a nucleotide sequence complementary to a first strand of a donor site sequence wherein the 3′ end of the LDG complementary to at least one of the nucleotides of a core sequence on the first strand of the donor site sequence; and
a right-donor guide (RDG) comprising, in the 5′ to 3′ direction, a nucleotide sequence that is a reverse complement to an opposite strand of the first strand of the donor site sequence wherein the 3′ end of the RDG is reverse complementary to at least one of the nucleotides of the core sequence on the opposite strand to the first strand of the donor site sequence;
wherein the donor site sequence is a polynucleotide sequence, and the core sequence of the donor site sequence is the same as the core sequence of the target site sequence.

43. The recombinant nucleic acid editing system of claims 12-41, wherein the target binding loop of the bridgeRNA comprises:
a left-target guide (LTG) comprising, in the 5′ to 3′ direction, a nucleotide sequence is reverse complementary to an opposite strand to a first strand of a target site sequence wherein the 3′ end of the LTG is complementary to at least one of the nucleotides of a core sequence on the opposite strand to the first strand of the target site sequence; and
a right-target guide (RTG) comprising, in the 5′ to 3′ direction, a nucleotide sequence that is a complementary to the first strand of the target site sequence wherein the 3′ end of the RTG is complementary to at least one of the nucleotides of the core sequence on the first strand of the target site sequence;
wherein the target site sequence is a polynucleotide sequence; and/or
wherein the donor binding loop of the bridgeRNA comprises:
a left-donor guide (LDG) comprising, in the 5′ to 3′ direction, a nucleotide sequence complementary to a first strand of a donor site sequence wherein the 3′ end of the LDG complementary to at least one of the nucleotides of a core sequence on the first strand of the donor site sequence; and
a right-donor guide (RDG) comprising, in the 5′ to 3′ direction, a nucleotide sequence that is a reverse complement to an opposite strand of the first strand of the donor site sequence wherein the 3′ end of the RDG is reverse complementary to at least one of the nucleotides of the core sequence on the opposite strand to the first strand of the donor site sequence;
wherein the donor site sequence is a polynucleotide sequence, and the core sequence of the donor site sequence is the same as the core sequence of the target site sequence.

44. The recombinant nucleic acid editing system of claim 42 or 43, wherein the target site sequence comprises sequence X₁X₂X₃X₄X₅X₆X₇X₈X₉X₁₀X₁₁X₁₂X₁₃X₁₄ where X is any nucleotide, and X₈X₉ are the core and one or more of X₁₂, X₁₃, and X₁₄ are optionally part of the target site sequence.

45. The recombinant nucleic acid editing system of claim 44, wherein the bridgeRNA encodes an LTG in the 5′ to 3′ direction X₁X₂X₃X₄X₅X₆X₇X₈ and an RTG in the 5′ to 3′ direction Y₁₄Y₁₃Y₁₂Y₁₁Y₁₀Y₉Y₈ where Y is the complementary nucleotide to X and one or more of Y₁₄, Y₁₃, and Y₁₂ are optionally part of RTG or wherein the bridgeRNA encodes an LTG in the 5′ to 3′ direction X₁X₂X₃X₄X₅X₆X₇X₈X₉ and an RTG in the 5′ to 3′ direction Y₁₄Y₁₃Y₁₂Y₁₁Y₁₀Y₉ where Y is the complementary nucleotide to X and one or more of Y₁₄, Y₁₃, and Y₁₂ are optionally part of RTG.

46. The recombinant nucleic acid editing system of claim 42 or 43, wherein the donor site sequence comprises sequence STIR-Xₙ₁—X₁X₂X₃X₄X₅X₆X₇X₈X₉X₁₀X₁₁X₁₂X₁₃X₁₄—X₁₂-STIR where X is any nucleotide, one or more of X₁₂, X₁₃, and X₁₄ are optionally part of the donor site sequence, STIR is optional, but if present is a sub-terminal inverted repeat comprising 2 to 20 nucleotides, and X₈X₉ are the core, and n1 and n2 can independently be zero to 10.

47. The recombinant nucleic acid editing system of claim 46, wherein the bridgeRNA encodes an LDG in the 5′ to 3′ direction X₁X₂X₃X₄X₅X₆X₇X₈ and an RDG in the 5′ to 3′ direction Y₁₄Y₁₃Y₁₂Y₁₁Y₁₀Y₉Y₈ where Y is the complementary nucleotide to X and one or more of Y₁₄, Y₁₃, and Y₁₂ are optionally part of RDG or wherein the bridgeRNA encodes an LDG in the 5′ to 3′ direction X₁X₂X₃X₄X₅X₆X₇X₈X₉ and an RDG in the 5′ to 3′ direction Y₁₄Y₁₃Y₁₂Y₁₁Y₁₀Y₉ where Y is the complementary nucleotide to X and one or more of Y₁₄, Y₁₃, and Y₁₂ are optionally part of RDG.

48. The recombinant nucleic acid editing system of claims 12-47, wherein the target site sequence is located on genomic DNA, a linear dsDNA, a dsDNA plasmid, ssDNA or RNA.

49. The recombinant nucleic acid editing system of claims 13-47, wherein the donor site sequence is located on genomic DNA, a linear dsDNA, a dsDNA plasmid, ssDNA or RNA.

50. The recombinant nucleic acid editing system of claims 48-49, wherein the dsDNA plasmid further comprises a polynucleotide sequence for insertion into the donor site sequence.

51. The recombinant nucleic acid editing system of claims 48-49, wherein the dsDNA plasmid further comprises a polynucleotide sequence for insertion into the target site sequence.

52. The recombinant nucleic acid editing system of claims 48-49, wherein the target site sequence and donor site sequence on the genomic DNA are located on the same DNA strand.

53. The recombinant nucleic acid editing system of claims 48-49, wherein the target site sequence and donor site sequence on the genomic DNA are located on different chromosomes.

54. The recombinant nucleic acid editing system of claims 12-53, wherein the bridgeRNA is a split bridgeRNA.

55. The recombinant nucleic acid editing system of claim 54, wherein the bridgeRNA comprises a first RNA molecule that comprises a first portion of the bridgeRNA and a second RNA molecule that comprises a second portion of the bridgeRNA.

56. The recombinant nucleic acid editing system of claim 55, wherein the first portion of the bridgeRNA comprises the internal loop comprising the first nucleotide sequence that is complementary to the first target site sequence of the target DNA and the second portion of the bridgeRNA comprises the second internal loop comprising the third nucleotide sequence that is complementary to the first donor site sequence of a donor DNA, and the fourth nucleotide sequence that is complementary to the second donor site sequence.

57. The recombinant nucleic acid editing system of claim 55-56, wherein the first portion of the bridgeRNA is encoded on a nucleic acid and is operably linked to a first promoter and the second portion of the bridgeRNA is encoded on the same or a different nucleic acid and is operably linked to a second promoter.

58. The recombinant nucleic acid editing system of claim 55-56, wherein the first portion of the bridgeRNA and second portion are encoded on a nucleic acid and further comprise one or more ribozyme cites which results in cleavage of the expressed bridgeRNA into the first and second RNA molecules.

59. The recombinant nucleic acid editing system of claims 42-58, wherein any of the LTG, RTG, LDG, and/or RDG of the bridgeRNA are complementary to more nucleotides their respective target site sequence or donor site sequence than the number of complementary nucleotides in its corresponding naturally occurring bridgeRNA.

60. The recombinant nucleic acid editing system of claim 59, wherein the total number of nucleotides comprising the target binding loop and/or donor binding loop of the bridgeRNA is the same as its corresponding naturally occurring bridgeRNA.

61. The recombinant nucleic acid editing system of claim 59, wherein the total number of nucleotides comprising the target binding loop and/or donor binding loop of the bridgeRNA is the increased as compared to its corresponding naturally occurring bridgeRNA.

62. The recombinant nucleic acid editing system of claims 42-58, wherein any of the LTG, RTG, LDG, and/or RDG of the bridgeRNA are not complementary to a nucleotide of the core sequence in their respective target site sequence or donor site sequence but are complementary to the same number of nucleotides in the respective target site sequence or donor site sequence as its corresponding naturally occurring bridgeRNA.

63. The recombinant nucleic acid editing system of claim 42 or 43, wherein the target site sequence comprises sequence X₋₁X₁X₂X₃X₄X₅X₆X₇X₈X₉X₁₀X₁₁X₁₂X₁₃X₁₄ where X is any nucleotide, and X₈X₉ are the core and one or more of X₁₂, X₁₃, and X₁₄ are optionally part of the target site sequence and wherein the bridgeRNA encodes an LTG in the 5′ to 3′ direction X₋₁X₁X₂X₃X₄X₅X₆X₇ and an RTG in the 5′ to 3′ direction Y₁₄Y₁₃Y₁₂Y₁₁Y₁₀Y₉Y₈ where Y is the complementary nucleotide to X and one or more of Y₁₄, Y₁₃, and Y₁₂ are optionally part of RTG.

64. The recombinant nucleic acid editing system of claim 42 or 43, wherein the donor site sequence comprises sequence STIR-X₋₁X₁X₂X₃X₄X₅X₆X₇X₈X₉X₁₀X₁₁X₁₂X₁₃X₁₄Xₙ₂-STIR where X is any nucleotide, one or more of X₁₂,X₁₃,and X₁₄ are optionally part of the donor site sequence, STIR is optional, but if present is a sub-terminal inverted repeat comprising 2 to 20 nucleotides, and X₈X₉ are the core, and n1 and n2 can independently be 1 to 10, and wherein the bridgeRNA encodes an LDG in the 5′ to 3′ direction X₋₁X₁X₂X₃X₄X₅X₆X₇ and an RDG in the 5′ to 3′ direction Y₁₄Y₁₃Y₁₂Y₁₁Y₁₀Y₉Y₈ where Y is the complementary nucleotide to X and one or more of Y₁₄, Y₁₃, and Y₁₂ are optionally part of RDG.

65. The recombinant nucleic acid editing system of claims 42-58, wherein one or more of the nucleotides of the LTG, RTG, LDG, and/or RDG of the bridgeRNA base pair to a nucleotide of their respective target site sequence or donor site sequence vis non-canonical base pairing.

66. A vector comprising any of the nucleic acids of the nucleic acid editing system of claims 1-65.

67. A host cell comprising any of the vector(s) of claim 66.

68. The recombinant nucleic acid editing system of claims 1-65, wherein any of the nucleic acids of the nucleic acid editing system further comprise an inducible promoter.

69. A method of integrating a DNA molecule of interest into a sequence specific site of a DNA of interest of a cell, the method comprising introducing into the cell: a nucleic acid editing system according to claims 1-65.

70. The method of claim 69, wherein the cell is a mammalian cell.

71. The method of claim 69, where the cell is a human cell.

72. The method of claims 69-71, wherein the DNA of interest of the cell comprises a donor site sequence and the DNA molecule of interest is part of a nucleic acid of the nucleic acid editing system comprising a target site sequence.

73. The method of claims 69-71, wherein the DNA of interest of the cell comprises a target site sequence and the DNA molecule of interest is part of a nucleic acid of the nucleic acid editing system comprising a donor site sequence.

74. The method of claim 72, wherein the DNA of interest of the cell further comprises a second donor site sequence and the DNA molecule of interest further comprises a second target site sequence and the nucleic acid editing system comprises a second bridgeRNA that targets the second donor site sequence and second target site sequence.

75. The method of claim 73, wherein the DNA of interest of the cell comprises a second target site sequence and the DNA molecule of interest further comprises a second donor site sequence and the nucleic acid editing system comprises a second bridgeRNA that targets the second donor site sequence and second target site sequence.

76. The method of claims 72-75, wherein the sequence of the bridgeRNA was engineered before introduction of the nucleic acid editing system to bind to the donor site sequence and target site sequence.

77. The method of claims 70-76, wherein the DNA of interest of the cell is the genome of the cell.

78. The method of claims 70-76, wherein the DNA of interest of the cell is a plasmid.

79. A method of inverting a DNA sequence of a DNA of interest of a cell, the method comprising introducing into the cell: a nucleic acid editing system according to claims 1-65, wherein a target site sequence and a donor site sequence are present on the same DNA molecule of interest and the LD of the donor site sequence and the RT of the target site sequence are on the same DNA strand.

80. The method of claim 79, wherein the DNA of interest of the cell is the genome of the cell.

81. The method of claims 79-80, wherein the sequence of the bridgeRNA was engineered before introduction of the nucleic acid editing system to bind to the donor site sequence and target site sequence.

82. A method of excising a DNA sequence of a DNA of interest of a cell, the method comprising introducing into the cell: a nucleic acid editing system according to claims 1-65, wherein a target site sequence and a donor site sequence are present on the same DNA molecule of interest and the LD of the donor site sequence and the LT of the target site sequence are on the same DNA strand.

83. The method of claim 82, wherein the DNA of interest of the cell is the genome of the cell.

84. The method of claims 82-83, wherein the sequence of the bridgeRNA was engineered before introduction of the nucleic acid editing system to bind to the donor site sequence and the target site sequence.

85. A method of translocating DNA sequences between two linear DNA molecules of interest, the method comprising introducing into a cell: a nucleic acid editing system according to claims 1-65, wherein a donor site sequence is present on a first linear DNA molecule and a target site sequence is present on a second linear DNA molecule.

86. The method of claim 85, wherein the linear DNA molecules of interest of the cell are chromosomes of the cell.

87. The method of claims 85-86, wherein the sequence of the bridgeRNA was engineered before introduction of the nucleic acid editing system to bind to the donor site sequence and the target site sequence.
